(19) United States
(12) Patent Application Publication
(10) Pub. No.: US 2004/0175817 A1
(43) Pub. Date: Sep. 9, 2004

(54) Crystallised catalytic domain of matrix metalloproteinase 9 (mmp9) and the use of its three dimensional structure to design mmp9 modulators

(76) Inventors: Holly Jepson, Macclesfield (GB); Claire Minshull, Macclesfield (GB); Richard Pauptit, Macclesfield (GB); Sian Rowsell, Macclesfield (GB)

Correspondence Address: FISH & RICHARDSON PC, 225 FRANKLIN ST, BOSTON, MA, 02110, US

(21) Appl. No.: 10/480,621

(22) PCT Filed: Jun. 24, 2002
(86) PCT No.: PCT/SE02/01266

(30) Foreign Application Priority Data
Jun. 27, 2001 (SE) 0102298-7

(51) Int. Cl. G06F 19/00; G01N 33/48; G01N 33/50; C12N 9/64

(52) U.S. Cl. 435/226; 702/19

(57) ABSTRACT

The invention provides a crystalline form of a polypeptide corresponding to the catalytic domain of a matrix metalloproteinase protein, MMP9. The active site binding region of the MMP9 protein is defined by its amino acid residues and their atomic coordinates. This three dimensional structure may be used to select or design chemical modulators or MMP9, particularly MMP9 inhibitors. These modulators may be used to treat a metalloproteinase mediated disease or condition.

### FIELD OF THE INVENTION

This invention relates to the crystallised catalytic domain of matrix metalloproteinase 9 (MMP9) and the use of its three dimensional structure to design MMP9 modulators.

### BACKGROUND OF THE INVENTION

Active proteins, such as enzymes, involved in physiological and pathological processes are important targets in the development of pharmaceutical compounds and treatments. Knowledge of the three dimensional (tertiary) structure of active proteins allows the rational design of mimics or modulators of such proteins. By searching structural databases of compounds using structural parameters derived from the active protein of interest, it is possible to select compound structures that may mimic or interact with these parameters. It is then possible to synthesise the selected compound and test its activity. Alternatively, the structural parameters derived from the active protein of interest may be used to design and synthesise a mimic or modulator with the desired activity. Such mimics or modulators may be useful as therapeutic agents for treating certain diseases. For example, WO98/07835 discloses crystal structures of a protein tyrosine kinase optionally complexed with one or more compounds. The atomic coordinates of the enzyme structures and any of the bound compounds are used to determine the three dimensional structures of kinases with unknown structure and to identify modulators of kinase functions. As another example, WO99/01476 discloses the crystal structures of anti-Factor IX Fab fragments (antibodies) and their use to identify and design new anticoagulant agents.

Knowledge of the three dimensional structure of an active protein is essential for the rational design of mimics or modulators of that protein. Lack of structural knowledge is a barrier to the development of new mimics or modulators that may have extremely useful pharmaceutical properties. The present invention relates to the previously unknown three dimensional structure of matrix metalloproteinase 9 (MMP9) and its use.

The matrix metalloproteinase (MMPs) are a family of structurally-related zinc-containing endopeptidases which mediate the breakdown of connective tissue macro-molecules. The mammalian MMP family is composed of at least twenty enzymes, classically divided into four sub-groups based on substrate specificity and domain structure (Alexander & Werb, 1991; Murphy & Reynolds, 1993; Birkedal-Hansen, 1995). The sub-groups are the collagenases (such as MMP1, MMP8, MMP13), the stromelysins (such as MMP3, MMP10, MMP11), the gelatinases (such as MMP2, MMP9) and the membrane-type MMPs (such as MMP14, MMP15, MMP16, MMP17). Enzyme activity is normally regulated in vivo by tissue inhibitors of metalloproteinases (TIMPs).

Because of their central role in re-modelling connective tissue, both as part of normal physiological growth and repair and as part of disease processes, there has been substantial interest in these proteins as targets for therapeutic intervention in a wide range of degenerative and inflammatory diseases, such as arthritis, atherosclerosis, and cancer (Whittaker et al, 1999). It was in the context of cell movement in malignancy that the gelatinases MMP2 and MMP9 were described, through their ability to degrade basement membranes as well as other matrix components (Liotta et al 1981; Collier et al 1988; Wilhelm et al 1989).

MMP9 (92 kDa Gelatinase) is a secreted protein which was first purified, then cloned and sequenced by Wilhelm et al (1989). The review of MMP9 by Vu & Werb (1998) provides an excellent source for detailed information and references on this protease. The expression of MMP9 is restricted normally to a few cell types, including trophoblasts, osteoclasts, neutrophils and macrophages. However, its expression can be induced in these same cells and in other cell types by several mediators, including exposure of the cells to growth factors or cytokines. These are the same mediators often implicated in initiating an inflammatory response. As with other secreted MMPs, MMP9 is released as an inactive Pro-enzyme or precursor which includes a propeptide domain. The Pro-enzyme is subsequently cleaved to form the active enzyme. The balance of active MMP9 versus inactive enzyme is further regulated in vivo by interaction with TIMP-1 (Tissue Inhibitor of Metalloproteinases-1), a naturally-occurring protein. TIMP-1 binds to the C-terminal region of MMP9, leading to inhibition of the catalytic domain of MMP9. The balance of induced expression of ProMMP9, cleavage of Pro- to active MMP9 and the presence of TIMP-1 combine to determine the amount of catalytically active MMP9 which is present at a local site. Proteolytically active MMP9 attacks substrates which include gelatin, elastin, and native Type IV and Type V collagens. It has no activity against native Type I collagen, proteoglycans or laminins.

Enzymes of the MMP9 and MMP2 subfamilies are similar in many ways. For example, their most distinctive catalytic property is a near absolute requirement for leucine at the substrate P1 position (protein S1 position). However, despite these similarities, MMP9 and MMP2 show a different substrate specificity. Thus MMP9 and MMP2 are clearly different enzymes although they are homologs (similar proteins having a certain degree of sequence identity but with distinct and different activity profiles).

Human MMPs are generally composed of three domains: the N-terminal propeptide domain, the protease or catalytic domain (the zinc-binding domain), and the C-terminal hemopexin-like domain. The gelatinases MMP2 and MMP9 also contain an additional domain composed of three fibronectin repeats inserted in tandem within the catalytic domain. The active site binding region lies within the catalytic domain and incorporates the S1′ pocket (also called the S1′ specificity pocket or the S1′ selectivity pocket) and the S1 pocket. It is known that the catalytic domain of certain MMPs is folded into a compact domain approximately 40×40×30 Å in size, consisting of a five-stranded β-sheet and three α-helices (Lovejoy et al. 1999; Morgunova et al. 1999; Dhanaraj et al. 1999; Moy et al. 2000; Zhang et al. 2000; Chen et al. 2000; Dhanaraj et al. 1996; Becker et al. 1995; Gooley et al. 1994; Van Doren et al. 1995; Borkakoti et al. 1994; Lovejoy et al. 1994; Lovejoy et al. 1994a; Spurlino et al. 1994; Browner et al. 1995; Moy et al. 1997; Moy et al. 1998; Moy et al. 1999; Stams et al. 1994; Finzel et al. 1998; Stockman et al. 1998; Pavlovsky et al. 1999; Gomis-Rüth et al. 1997 Li et al. 1995; Reinemer et al. 1994; Bode et al. 1994; Grams et al. 1995; Grams et al. 1995a; Esser et al. 1997). However the three dimensional structure of MMP9 has not previously been disclosed.

The largest structural differences within the catalytic domains of various MMPs are the conformation of surface loops. The S1′ specificity pocket has been described for certain MMPs, including MMP2 (Dhanaraj et al. 1999), MMP13 (Lovejoy et al. 1999), stromelysin (Dhanaraj et al. 1996), fibroblast collagenase and matrilysin (Lovejoy et al. 1999; Lovejoy et al. 1994; Lovejoy et al. 1994a; Browner et al. 1995). Among members of the MMP family, the S1′ loops do not share amino acid sequence similarity and also differ in length. The S1′ loop has no regular secondary structure in any MMP for which a structure has been determined. Thus, both the structural variability and the sequence variability of the S1′ loops contribute to the variation in overall size and shape of the S1′ pockets in the different MMPs.

There is a growing body of data implicating roles for MMP9 in various physiological and pathological processes. Physiological roles include the invasion of embryonic trophoblasts through the uterine epithelium in the early stages of embryonic implantation, some role in the growth and development of bones, and migration of inflammatory cells from the vasculature into tissues. Increased MMP9 expression has been observed in certain pathological conditions, thereby implicating MMP9 in disease processes such as asthma, arthritis, tumour metastasis, Alzheimer's, Multiple Sclerosis, and atherosclerosis.

Whilst the importance of the MMP family in the pathogenesis of arthritis and cancer has been well documented over many years, it is only relatively recently that MMPs and their inhibitors have been shown also to be important in connective tissue re-modelling in diseases of the cardiovascular system, such as atherosclerosis (Henney et al, 1991; Galis et al, 1994; Dollery et al, 1995). Various members of the MMP family have been shown to be expressed in atherosclerotic lesions of various types, but MMP9 is consistently seen in inflammatory atherosclerotic lesions, typically expressed by lipid laden macrophages. Evidence from histological investigations and molecular genetic studies implicates MMP9 over-expression in the vascular re-modelling events preceding plaque rupture, the most common cause of acute myocardial infarction (Brown et al, 1995). More recently, animal studies have shown that reducing MMP9 activity, either by genetic manipulation or through pharmacological intervention, has an impact on ventricular re-modelling following infarction and as such may represent a key mechanism in the pathogenesis of heart failure (Rhode et al 1999).

A number of MMP inhibitor compounds are known and some are being developed for pharmaceutical uses (see for example the review by Beckett & Whittaker, 1998). Different classes of compounds may have different degrees of potency and selectivity for inhibiting various MMPs. Whittaker M. et al (1999) review a wide range of known MMP inhibitor compounds. They state that an effective MMP inhibitor requires a zinc binding group or ZBG (functional group capable of chelating the active site zinc(II) ion), at least one functional group which provides a hydrogen bond interaction with the enzyme backbone, and one or more side chains which undergo effective van der Waals interactions with the enzyme subsites. Zinc binding groups in known MMP inhibitors include hydroxamates, reverse hydroxamates, thiols, carboxylates and phosphonic acids.

The structures of several hydroxamate-MMP complexes have been determined (Dhanaraj et al. 1996; Lovejoy et al. 1999; Li et al., 1995; Spurlino et al. 1994; Reinemer et al. 1994; Bode et al. 1994; Grams et al. 1995; Grams et al. 1995a; Browner et al. 1995; Stams et al., 1994; Van Doren et al. 1995; Borkakoti et al. 1994), including an MMP2-hydroxamate complex (Dhanaraj et al. 1999) and two recent solution structures of the catalytic fragment of human collagenase-3 (MMP13) complexed with a non-peptidic sulphonamide hydroxamic acid inhibitor (Moy et al. 2000; Zhang et al. 2000). However there has been no structural analysis of an MMP bound to a reverse hydroxamate. Reverse hydroxamate inhibitors may have better pharmaco-kinetic properties than their hydroxamate counterparts and therefore may present a more useful starting point for drug design.

There has been some investigation of MMP mutants showing that specific mutations do not affect the shape of the active site (Steele et al. 2000). For example, Morgunova et al (1999) designed an MMP2 mutant (Glu404Ala) to prevent autoproteolyis, preserving the intact propeptide (pro-MMP2), and the mutation had no influence on the architecture of the active site.

Chen et al. 2000 used the high-resolution NMR solution structure of the catalytic fragment of MMP13 (human collagenase-3) as a starting point for structure-based design of selective inhibitors for MMP13. Skiles et al (2001) reviewed the design, structure and therapeutic application of MMP inhibitors. They also discussed the structural characteristics of certain MMP enzymes and the binding of inhibitors to these enzymes. However the three dimensional structure of MMP9 is not known and is difficult to elucidate. No-one has been able to obtain crystals of any part of MMP9 of sufficient quality for determining the three dimensional structure of the catalytic domain and active site of MMP9.

We have now overcome the difficulties to produce crystals of the MMP9 catalytic domain that are of sufficient quality to determine the three dimensional structure of the protein by X-ray diffraction methods. There is a clear need for this MMP9 structural information to enable identification and structure-based design of new MMP9 modulators (preferably inhibitors) for the treatment of various diseases or conditions.

### BRIEF DESCRIPTION OF THE DRAWINGS

FIG. 1 is a schematic representation of the three dimensional structure of the wild type MMP9:reverse hydroxamate complex.

FIG. 2 is a stereo diagram of the wild type MMP9:reverse hydroxamate complex.

FIG. 3 is a diagram showing a close-up of the MMP9 mutant (E402Q):reverse hydroxamate complex together with a portion of the (2Fo-Fc) electron density map.

FIG. 4 is a diagram showing superposition of the MMP9 mutant (E402Q) and wild type MMP9 active sites.

FIG. 5 is a Grasp representation of the MMP9 active site binding region with bound ligand.

FIG. 6 is a schematic represetation of the three dimensional structure of MMP2 oriented to maximize alignment with the structure of the MMP9:reverse hydroxamate complex shown in FIG. 1.

FIG. 7 shows the aligned amino acid sequences of MMP9 (SEQ ID NO 2) and MMP2 (SEQ ID NO 1).

### DESCRIPTION OF THE INVENTION

According to a first aspect of the invention, we provide an MMP9 crystal. In particular we provide a crystalline form of a polypeptide corresponding to the catalytic domain of an MMP9 protein. The catalytic domain may be found within the complete MMP9 protein or within a fragment of the MMP9 protein. The catalytic domain may be derived from a wild type MMP9 protein or from an MMP9 mutant or variant. A mutant is a wild type MMP9 protein having one or more changes in its amino acid sequence. An MMP9 mutant may have the same activity as the wild type protein, may have a modified activity or may be inactive. A variant is a wild type or mutant MMP9 protein having one or more portions of its amino acid sequence removed, so that the variant is a different length to the wild type or mutant protein. A variant usually has the same activity as the original wild type or mutant MMP9.

The invention provides crystals of sufficient quality to determine the three dimensional structure to high resolution of any portion of the MMP9 catalytic domain.

According to a further aspect of the invention, we provide a crystalline form of a polypeptide corresponding to the catalytic domain of an MMP9 protein in complex with an MMP9 inhibitor compound. For example, the MMP9 inhibitor compound may have a reverse hydroxamate zinc binding group. Such reverse hydroxamates include, for example, compounds of Formula I:

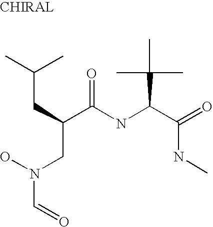

Using X-ray crystallography we have determined the three dimensional molecular structure of an MMP9 catalytic domain. We have also determined the unique shape of an MMP9 active site binding region (defined by the atomic coordinates of its amino acids). Further, we have determined the spatial arrangement of an MMP9 inhibitor relative to the MMP9 active site binding region. The structural information can be stored on a computer-readable medium and may be used for rational drug design.

One of the major hurdles in the crystallisation of multidomain proteins is their flexibility. To increase the chances of crystallising MMP9, we used a variant construct (an enzyme construct limited to the catalytic domain with the fibronectin domain excised). This provided a rigid and compact domain.

Another major hurdle in the crystallisation and structure determination of MMP9 is that the MMP9 catalytic domain is aggressively autolytic. We therefore purified wild-type MMP9 in the presence of an inhibitor and then crystallised the purified complex. Addition of an inhibitor during the refolding step following urea denaturation is essential for prevention of N-terminal degradation and the formation of well-ordered crystals.

Weak inhibitors such as acetohydroxamic acid prevent autodegradation of the wild type MMP9 protein during refolding. However we found that weak inhibitors are not sufficiently potent to prevent autodegradation of the wild type MMP9 protein during storage or during the period of crystallisation, rendering crystallographic studies on such complexes extremely difficult. In order to form crystals of complexes with wild type protein, it is necessary to replace the weak inhibitor with a much more potent inhibitor (such as a compound of Formula I) during the final purification step before crystallisation. This limits the usefulness of the wild type construct for rational drug design using less potent inhibitors.

To overcome the problems and limitations, we designed an inactive mutant of the MMP9 catalytic domain in which the essential active site glutamate (amino acid residue E) was mutated to glutamine (amino acid residue Q). The MMP9 mutant is known as E402Q and is inactive. Use of the more stable mutant for crystallisation prevents autodegradation. It also enables stable MMP9 complexes to be crystallised with weak inhibitors by co-crystallisation. It should also be possible to crystallise the mutant protein without the inhibitor, followed by soaking of the crystals with weak or strong inhibitors.

We determined the three dimensional structure of the catalytic domain of wild type MMP9 in complex with a peptidic reverse hydroxamate inhibitor (compound of Formula I) by X-ray diffraction analysis at a nominal resolution of 2.3 Å. We also determined the three dimensional structure of the catalytic domain of the inactive MMP9 mutant (E402Q) in complex with the same inhibitor at a nominal resolution of 2.1 Å. The E to Q mutation does not change the three dimensional structure of the MMP9 enzyme.

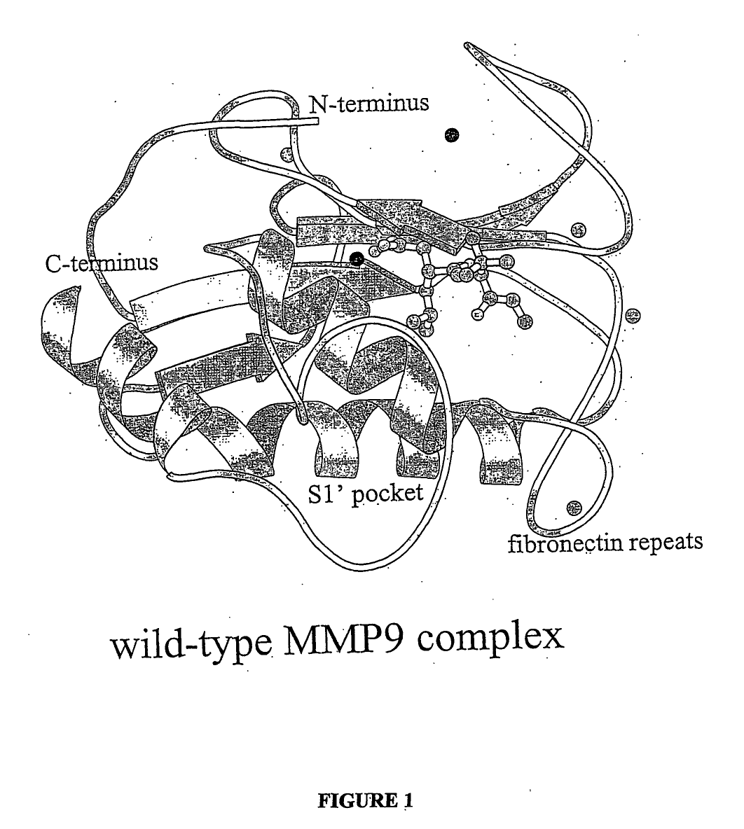

FIG. 1 is a schematic representation of the three dimensional structure of the wild type MMP9:reverse hydroxamate complex. The S1′ specificity pocket and the region from where the three fibronectin repeats were deleted are indicated. The inhibitor is shown in all-atom representation, zinc and calcium ions are represented by dark grey and light grey spheres respectively. FIG. 1 was generated using the programs BOBSCRIPT (Esnouf 1997) and RASTER3D (Bacon & Anderson 1998; Merritt & Murphy 1994).

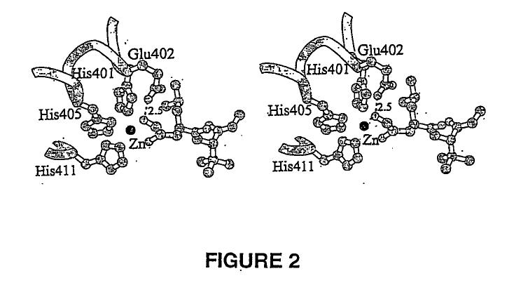

FIG. 2 is a stereo diagram of the wild type MMP9:reverse hydroxamate complex. It shows some of the interactions between the bound peptidic reversed hydroxamate inhibitor (compound of Formula I) and MMP9. A short hydrogen bond (2.5 Å) is formed between Glu402 and the inhibitor.

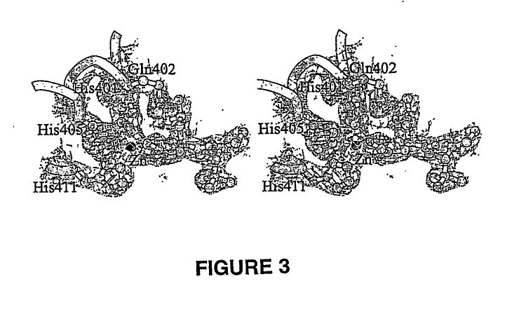

FIG. 3 is a diagram showing a close-up of the MMP9 mutant (E402Q):reverse hydroxamate complex together with a portion of the (2Fo-Fc) electron density map.

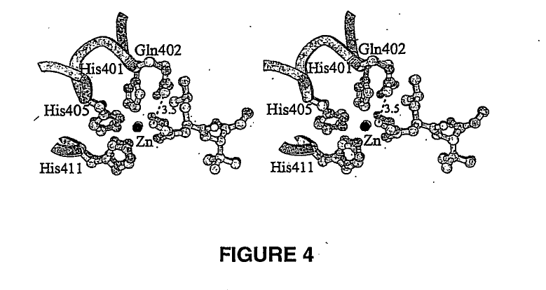

FIG. 4 is a diagram showing superposition of the MMP9 mutant (E402Q) and wild type active sites. The mutant structure is coloured light grey; the wild type structure is coloured dark grey. The structure is perturbed little on introduction of the mutant. The short hydrogen bond to the inhibitor seen in the wild type complex is absent in the mutated structure (the corresponding atoms are 3.5 Å apart in one of the molecules in the crystal asymmetric unit; in the second molecule, Oε of Gln402 is only 3.2 Å away from O1 of the inhibitor). FIG. 4 was generated using BOBSCRIPT (Esnouf 1997).

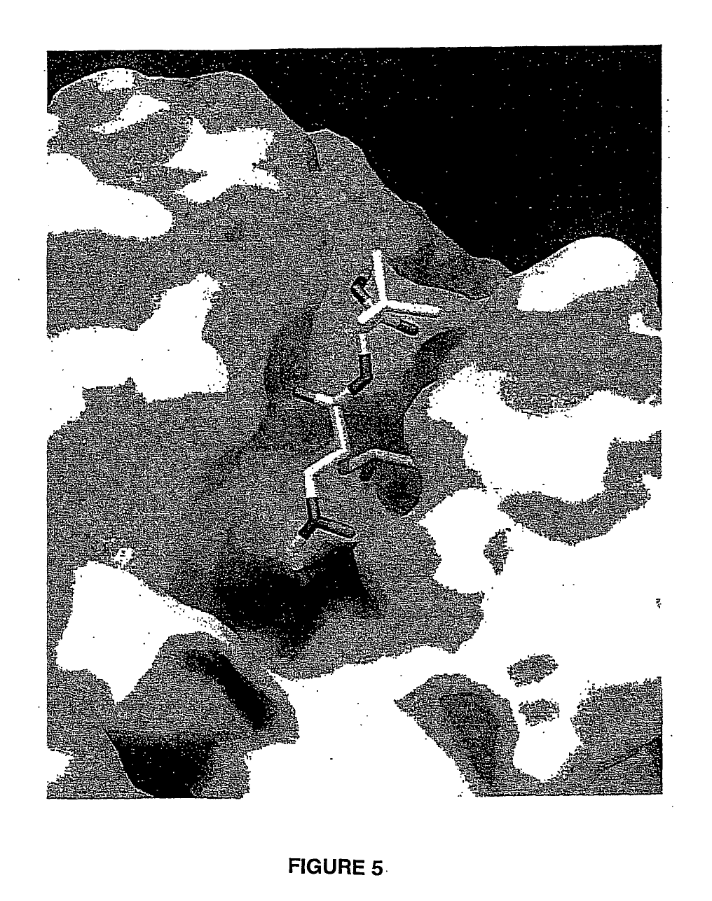

FIG. 5 is a Grasp representation of the MMP9 active site binding region with bound ligand (reverse hydroxamate compound of Formula I). FIG. 5 was generated using the program GRASP (Nicholls 1991).

The catalytic domain of MMP9 (minus the fibronectin repeats) is folded into a compact domain approximately 40×40×30 Å in size, consisting of a five-stranded β-sheet and three α-helices as found for other MMPs.

From the three dimensional structure determined for MMP9, we have found that the active site binding region in MMP9 is uniquely defined by the atomic co-ordinates of its constituent amino acid residues. These amino acid residues are listed in Table 1.

TABLE 1

Phe110, Glu111, Tyr179, Pro180, Asp185, Gly186,
Leu187, Leu188, Ala189, His190, Ala191, Phe192,
Pro193, Lys214, Gly215, Gln391, Gly392, Tyr393,
Leu397, Val398, His401, Glu402, His405, Gly408,
Leu409, Asp410, His411, Pro415, Glu416, Ala417,
Leu418, Tyr420, Pro421, Met422, Tyr423, Arg424,
Phe425, Thr426, Glu427, Gly428, Pro429, Pro430,
Leu431, His432, and Lys433.

In MMP9 the catalytic centre is composed of the active-site zinc and the essential glutamate. The residues which chelate the catalytic zinc are His401, His405, and His411. The catalytic glutamate is Glu4O2. The numbering refers to the sequential numbering of the full-length human pro-MMP9 as defined in the Swissprot protein sequence database under accession number P14780.

The atomic coordinates for wild type and the MMP9 mutant (E402Q) are shown in Tables 2 and 3 at the end of this docunent. The Tables also give the atomic coordinates of the inhibitor compound in complex with the enzyme.

The term “atomic co-ordinates” refers to mathematical co-ordinates corresponding to the positions of every atom derived from mathematical equations related to the diffraction patterns obtained from a monochromatic beam of X-rays illuminating a crystal. The diffraction data are used to calculate an electron density map of the repeating unit of the crystal. The electron density maps are used to establish the positions of the individual atoms within the unit cell of the crystal. The term “unit cell” refers to the basic building block from which the entire volume of a crystal may be constructed. Those of skill in the art understand that a set of atomic co-ordinates determined by X-ray crystallography is not without experimental variation.

Table 2 lists the atomic coordinates in Protein Data Bank (PDB) format of the wild type MMP9 construct in complex with the reverse hydroxamate compound of Formula I. Table 3 lists the atomic coordinates in Protein Data Bank (PDB) format of the MMP9 mutant (E402Q) in complex with the reverse hydroxamate compound of Formula I. In both Tables the atomic coordinates are listed in those lines that begin with the code ATOM or HETATM, one atom per line. Following the code are: the unique atom number; the atom name; the amino acid residue name; the protein chain identifier; the amino acid residue number; the atomic coordinates x, y, and z in orthogonal Angstrom space; the atomic occupancy factor; the atomic temperature factor; the chain identifier; and the atom type. The atomic coordinates of the inhibitor compound carry the residue name of FRA. Solvent water molecules carry the residue name of HOH.

It is possible to reproduce the shape of the MMP9 active site binding region through carrying out similar structure determinations with minor changes in the experimental conditions (including changes in crystallisation conditions, crystal form, construct, etc). It will be appreciated that the atomic coordinates of the MMP9 active site binding region may vary within certain limits due to experimental variation. Such variation includes standard error (coordinates determined for the same construct may vary somewhat, for example Within 0.3 Å) and other variation (for example, coordinates of MMP9 mutants or variants). Therefore the shape of the active site binding region is defined by the set of all possible structures that contain C-alpha (Ca) atomic coordinates within 1.5 Å of the C-alpha positions of the MMP9 active site binding region residues defined above, when the two protein structures are superposed (placed on a common frame). The criterion of 1.5 Å is selected as appropriate because it is large enough to allow for experimental variation whereas it is small enough to discriminate between MMP9 and the most similar homolog MMP2.

According to a further aspect of the invention, we provide a crystalline form of a polypeptide corresponding to the active site binding region of an MMP9 protein wherein the active site binding region amino acid residues are identical or equivalent to those listed in Table 1 and the shape of the active site binding region is defined by the atomic coordinates given in Table 2 or Table 3 or by equivalent coordinates.

An amino acid residue is equivalent to a residue listed in Table I if it occurs within an MMP9 protein (including mutants and variants) at a position listed in Table 1.

Equivalent coordinates are those containing C-alpha (Cα) atomic coordinates within 1.5 Å of the C-alpha coordinates of the MMP9 active site binding region residues defined in Tables 2 or 3, when the polypeptide structures are superposed. Those skilled in the art will recognise the C-alpha positions in the active site binding region of the MMP9 protein particular positions of the amino acid side chains on the main protein chain). If the atomic coordinates of any particular C-alpha varies more than 1.5 Å from that defined in Tables 2 or 3, the protein structure does not have equivalent coordinates.

The invention provides the first structure determination of an MMP bound to a reverse hydroxamate (compound of Formula 1). The reverse hydroxamate inhibitor forms a short complementary strand similar to the known peptidic hydroxamate inhibitor complexes of other MMPs and binds the catalytic zinc in a similar manner to both the peptidic and non-peptidic hydroxamate inhibitor complexes of other MMPs. The structure we have determined includes the three dimensional coordinates of the reverse hydroxamate inhibitor giving previously unknown information about its spatial orientation in the MMP9 active site and details of interactions between the reverse hydroxamate inhibitor and MMP9.

In the wild type MMP9-reverse hydroxamate inhibitor complex, the inhibitor spans the S1 and S1′ pockets. Four hydrogen bonds are formed between the inhibitor and the protein main chain (interactions with the backbone amides of Leu189, Tyr423, and the carbonyl oxygen atoms of Pro421, Gly187). The side-chain of P1 leucine of the peptidic inhibitor is located in a large S1 pocket; the tertiary butyl group points out to solvent. The co-ordination of the catalytic zinc by His401, His405 and His411 is completed by both hydroxamate oxygen atoms of the inhibitor, in a distorted penta-coordinate geometry. One of the zinc-chelating oxygen atoms (O1) forms a short (2.5 Å) hydrogen bond with Glu402. Oxygen O1 also interacts with His405 and has a water-mediated interaction with the backbone amide of Ala192 (water molecule only visible in the electron density for one of the protein molecules in the crystal asymmetric unit). The other chelating oxygen (O2) interacts with His411 and with the carbonyl of Pro421 via a water molecule (water molecule only visible in the electron density for one of the protein molecules in the crystal asymmetric unit).

The structure of the MMP9 mutant catalytic domain shows no significant deviation from the wild type structure. The same reverse hydroxamate inhibitor was used in the wild-type and mutant complexes to show that the active site shape is not changed on introduction of the mutation. The only minor difference in structure is that the distance between Glu402 and the hydroxamate moiety in the mutant complex is too long to be considered hydrogen-bonding distance. Apart from this small discrepancy, the wild type and mutant MMP9 structures are identical.

The MMP9 protein is similar to the MMP2 protein in tertiary and quaternary structure as well as in some features associated with its catalytic activity. In addition these two proteins interact with inhibitors in very similar ways in the S1 pocket. However the active site binding region (S1′ pocket) of MMP9 is strikingly different from that of MMP2 in size and chemical composition. This difference provides a structural basis for understanding the difference in specificity between the two enzyme types.

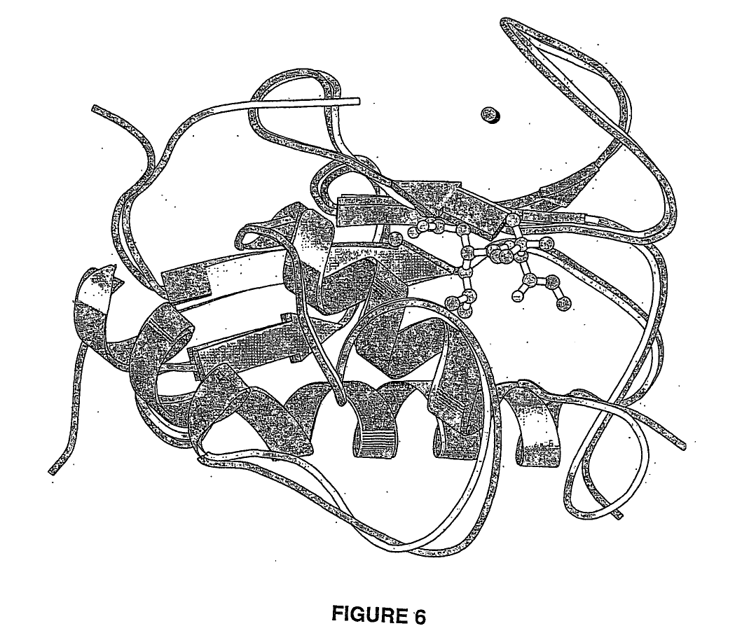

FIG. 6 is a schematic representation showing the three dimensional structure of MMP2 oriented to maximize alignment with the structure of the MMP9:reverse hydroxamate complex shown in FIG. 1. FIG. 6 shows superposition of the MMP9 and MMP2 catalytic domain structures (using deposited co-ordinates 1QIB, Dhanaraj et al. 1999). MMP9 is coloured light grey; MMP2 is coloured dark grey. The MMP9 inhibitor is shown in all-atom representation. The two zinc ions per catalytic domain are represented by light (MMP9) and dark (MMP2) spheres. The two structures are similar with the only significant differences being in the S1′ specificity pocket and in the region of the fibronectin deletions. Differences observed at the N-terminus are unlikely to be significant as the N-terminus of MMP9 is closer to the intact pro-MMP2 structure in this region (Morgunova et al. 1999).

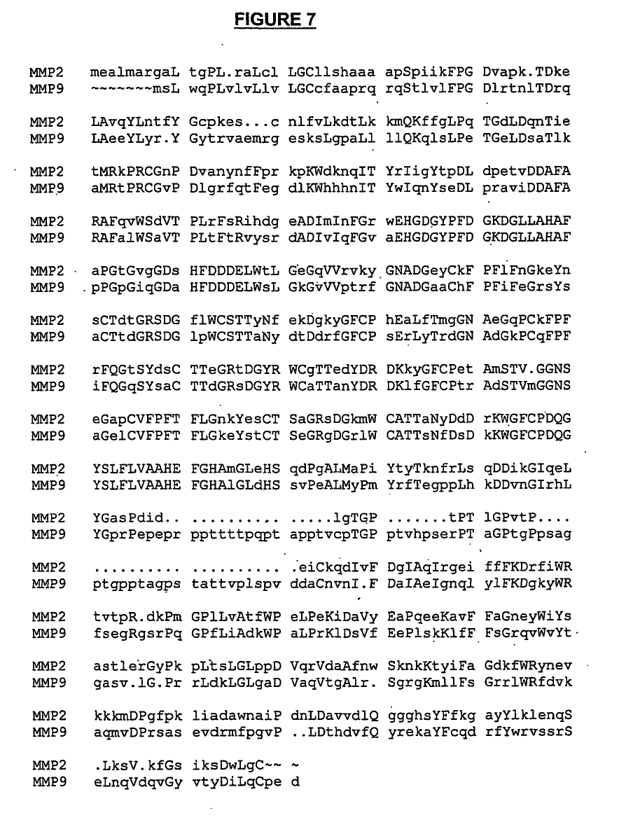

FIG. 7 shows the amino acid sequences of MMP9 (SEQ ID NO 2) and MMP2 (SEQ ID NO 1), aligned by PILEUP—capital letters indicate identity of amino acid residue between MMP2 and MMP9. Amino acids are abbreviated by one letter codes:

| | | |
|---|---|---|
| A = ALA = alanine | C = CYS = cysteine | D = ASP = aspartate |
| E = GLU = | F = PHE = phenylalanine | G = GLY = glycine |
| glutamate | | |
| H = HIS = histidine | I = ILE = isoleucine | K = LYS = lysine |
| L = LEU = leucine | M = MET = methionine | N = ASN = asparagine |
| P = PRO = proline | Q = GLN = glutamine | R = ARG = arginine |
| S = SER = serine | T = THR = threonine | V = VAL = valine |
| W = TRP = | Y = TYR = tyrosine. | |
| tryptophan | | |

Both the MMP9 and the MMP2 are full-length human sequences: each is the sequence of the Pro-enzyme including the propeptide domain. The MMP9 sequence is derived from lung tissue, from normal alveolar macrophages. An identical MMP9 sequence is found in granulocytes and in lung fibroblasts. The MMP2 sequence is derived from normal skin fibroblasts.

When the MMP9 and MMP2 structures are aligned on 148 residues with similar secondary structure or conformation, the average difference between equivalent alpha carbon atoms is 0.64 Å. The structures of MMP9 and MMP2 are therefore similar but significant differences occur in the S1′ specificity pocket. Residues 425-431 in MMP9 (427-433 for MMP2, numbered as for the intact MMP2 in Morgunova et al. 1999) form a loop which deviates from the equivalent loop in the MMP2 structures. The S1′ pocket in MMP9 and MMP2 may be described as a tunnel leading out to solvent, but the exact shape of the pocket in MMP9 is unique. Arg424 in MMP9 narrows the end of the S1′ pocket, pointing away from the pocket; the corresponding residue in MMP2 is a threonine (426).

According to a further aspect of the invention we provide a method to determine or design the three dimensional structure of a crystal form of MMP9 (including MMP9 mutants, variants, and co-complexes) by using a particular MMP9 catalytic domain structure. The atomic co-ordinates of a first MMP9 crystal may be used to model the structure of a second MMP9 crystal by difference Fourier or molecular replacement.

The term “molecular replacement” refers to a method that involves generating a preliminary model of a crystal whose atomic coordinates are not known, by orienting and positioning a related molecule whose atomic coordinates are known. Phases are then calculated from this model and combined with observed amplitudes to give an approximate Fourier synthesis of the structure whose coordinates are unknown.

The second crystal may be a crystal of a mutant, variant, or co-complex of MMP9. Preferably the active site binding region of the first MMP9 crystal is identical or equivalent to that defined by Tables 1 (amino acid residues) and Tables 2 or 3 (atomic coordinates). The shape of the MMP9 active site binding region in the second crystal model is an equivalent shape to that of the first. Equivalent shape is defined as a difference of up to 1.5 Å between each pair of matching Cα atoms for each residue contributing to the active site binding region. In other words, the positions of the Cα carbon atoms of the constituent residues of the active site binding region are within 1.5 Å when the first and second crystal structures are superposed.

So the invention provides a method to determine or design the three dimensional structure of a crystal form of MMP9 by difference Fourier or molecular replacement, using the atomic coordinates of a first MMP9 crystal to model the structure of a second MMP9 crystal wherein the active site binding region amino acid residues of the first MMP9 crystal are identical or equivalent to those listed in Table 1 and the shape of the active site binding region of the first MMP9 crystal is defined by the atomic coordinates given in Table 2 or Table 3 or by equivalent coordinates.

The method may be carried out as follows. An MMP9 protein (wild type, mutant or variant) is purified and crystallised as a pure protein or as a co-complex with an inhibitor compound. This crystal may have the same crystal form (same protein packing) as the crystal structure defined in Tables 2 or 3, or it may have a different crystal form (different protein packing). By taking some measurements of the crystal and using the atomic coordinates in Tables 2 or 3 (or equivalent coordinates), it is possible to work out the structure of the crystal by the known methods of difference Fourier (for the same packing) or molecular replacement (for different packing).

The invention further provides MMP9 proteins (including mutants and variants) designed by the above method. The MMP9 proteins may have identical properties to wild type MMP9 or may have one or more different properties compared to wild type MMP9 (for example, they may be more active mutants or inactive mutants).

According to a further aspect of the invention, we provide a method to select or design chemical modulators (preferably inhibitors) of MMP9 by using the MMP9 catalytic domain structure (including that of mutants, variants, and co-complexes) and the shape of the active site binding region (or an equivalent shape as previously defined). Information from the three dimensional atomic coordinates of the reverse hydroxamate inhibitor and its spatial orientation in relation to the three dimensional atomic coordinates of the MMP9 catalytic domain is used as a tool to design MMP9 modulators (preferably inhibitors). Small-molecule modulators of MMP9 may be selected or designed to fit into the shape of the active site binding region.

An MMP9 modulator may be selected by:

a) searching a structural database of compounds using parameters derived from the structure of the MMP9 active site binding region identical or equivalent to that defined by Tables 1 (amino acid residues) and Tables 2 or 3 (atomic coordinates); and

b) selecting a compound structure that may mimic or interact with these parameters.

It is then possible to synthesise the selected compound and test its activity.

An MMP9 modulator may be designed by taking parameters derived from the structure of the MMP9 active site binding region identical or equivalent to that defined by Tables 1 (amino acid residues) and Tables 2 or 3 (atomic coordinates) and using these parameters to model a compound structure that may mimic or interact with these parameters. The starting point for design may be the structure of a known weak inhibitor compound that can be modelled to an improved inhibitor compound using the MMP9 structural parameters. The designed compound may then be synthesised and tested.

The MMP9 mutant (E402Q) construct is a particularly useful tool for iterative drug design. Since the mutant protein is inactive, it can be stored in the absence of any inhibitor and subsequently be used for standard co-crystallisation procedures or crystal soaking procedures with both potent inhibitors and weaker binders.

Knowledge of the structural determinants that account for the difference in substrate specificity between MMP9 and MMP2 and other MMPs provides a foundation for the design of highly specific modulators of the MMP9 enzyme. Structural differences at the S1′ pocket between MMP9 and other MMPs (defined by differences in the atomic coordinates of residues in the S1′ pocket) may be used to design selective MMP9 modulators.

So the invention provides a method to select or design a chemical modulator of MMP9 by selecting or designing a modulator with a three dimensional structure that fits into the MMP9 active site binding region, wherein the active site binding region amino acid residues are identical or equivalent to those listed in Table 1 and the shape of the active site binding region is defined by the atomic coordinates given in Table 2 or Table 3 or by equivalent coordinates.

As described above, the MMP9 crystal structure may be used in the rational design of drugs which modulate (preferably inhibit) the action of MMP9. These MMP9 modulators may be used to prevent or treat the undesirable physical and pharmacological properties of MMP9 activity.

The invention provides modulators of MMP9 selected or designed by the above method. These modulators particularly inhibitors) may be useful as therapeutic agents to treat undesirable properties of MMP9 activity in humans.

Therefore in a further aspect, the present invention provides a modulator of MMP9 selected or designed by the above method or a pharmaceutically acceptable salt or in vivo hydrolysable ester thereof suitable for use in a method of therapeutic treatment of the human or animal body. In particular we disclose use in the treatment of a disease or condition mediated by MMP9.

In yet a further aspect the present invention provides a method of treating a metalloproteinase mediated disease or condition which comprises administering to a warm-blooded animal a therapeutically effective amount of a modulator of MMP9 or a pharmaceutically acceptable salt or in vivo hydrolysable ester thereof. The modulator of MMP 9 is selected or designed by the method described above. Metalloproteinase mediated diseases or conditions include: tumour growth and metastasis in cancer, inflammatory diseases in general, such as arthritis and osteoarthritis; atherosclerosis; aneurysmal disease; ventricular remodelling and heart failure; restenosis; periodontitis; neurodegenerative and neuroinflammatory diseases such as multiple sclerosis and Guillain Barré Syndrome; glomerulonephritis; blood-brain barrier leakage; breakdown in stroke and meningitis; occular autoimmune disease such as uveoretinitis; graft-versus-host disease; and non-insulin-dependant diabetes.

The present invention will now be described with reference to the following non-limiting Examples.

### EXAMPLE 1

Production of the Catalytic Domain of MMP9

The catalytic domain of human MMP9 was cloned so that the fibronectin type II-like domains which occur as an insert within the catalytic domain sequence were deleted (Shipley et al 1996). The remaining catalytic domain fragment containing residues 107-216 was fused to residues 391-443 by overlapping PCR The 5′ primer introduced an ATG start codon directly upstream of the phenylalanine (107) and a stop codon was introduced, via the 3′ primer, after residue 443 to prevent translation of the hemopexin-like domains. An inactive mutant of this domain was created by site directed mutagenesis, such that the glutamate (GAG) at position 402 of the full-length CDNA was mutated to give a glutamine (CAG). The product was cloned into the Ndel and Xhol sites of a T7 expression vector and transformed into E. coli BL21(DE3).This was grown up to log phase and induced with 0.4 mM IPTG for 4 hours to express the 18 kDa MMP9 catalytic domain protein

Following expression in E. coli, MMP9(107-216,391-443) is mainly located in the inclusion body fraction. The E. coli were harvested, washed and lysed and centrifuged to isolate the insoluble protein. The insoluble fraction was then suspended in 6M Urea to solubilise the protein. To generate active MMP9(107-216,391-443) the solubilised material was dialysed sequentially versus 4M, 1M and 0M Urea. Crystallization of this material after purification resulted in disordered crystals and analysis of the protein using mass spectrometry indicated heterogeneity at the N-terminus. It was found that the addition of acetohydroxamic acid in the refold buffers inhibited auto-proteolysis of the N-terminus of the protein. When crystallization of the protein was carried out it was found that where no acetohydroxamic acid was added during refolding disordered crystals formed whereas when acetohydroxamic was added the crystals were well ordered. Post dialysis, the protein was subjected to zinc chelate chromatography which selectively bound the MMP9 catalytic domain. A further chromatography step was then carried out using the tripeptide Pro-Leu-Gly bound to sepharose which bound correctly folded MMP9 catalytic domain. The protein was then eluted by addition of an MMP9 inhibitor. Expression and refolding of the [E402Q]MMP9(107-216,391-443) construct was carried out as for the wild type construct except that no acetohydroxamic acid was added to the refold buffers. After refolding, the enzyme was purified via zinc chelate chromatography followed by a gel filtration step.

### EXAMPLE 2

Purification of MMP9 Catalytic Domain

This method involves first the dialysis of inclusion bodies versus 4M, 2M, 1M and 0M Urea to generate active enzyme. The P9(107-216,391-443) present is then separated from impurities by zinc chelate chromatography on a Chelating Sepharose Fast Flow chromatography column charged with 0.1M zinc acetate. Further purification is then carried out by binding MMP9(107-216,391-443) to an NHS Activated Sepharose chromatography column bound with the peptide Pro-Leu-Gly. The MMP9(107-216,391-443) present is eluted by the addition of 0.5 mM MMMP9 Inhibitor. Mass spectrometry and N-terminal sequencing indicate the product to be MMP9(107-216,391-443) with an extra Met at the N-termini. The product is shown to possess MMP activity via-FRET and Zymographic analysis. Post refolding, the mutated enzyme was purified via zinc chelate chromatography followed by a gel filtration step.

### EXAMPLE 3

Crystallisation of MMP9 Catalytic Domain

The MMP9:reverse hydroxamate inhibitor complexes were crystallised at 15° C. by hanging-drop vapour diffusion. For crystallization of the wild type complex, the enzyme-inhibitor complex was purified as detailed above. The crystallisation drops contained a 1:1 mixture of purified complex solution (0.55 mg/ml protein and 0.5 mM inhibitor solution concentrated to ˜4 mg/ml in 20 mM Tris-HCl pH 7.5, 2 mM CaCl₂, 50 mM NaCl) and reservoir buffer (3.6M NaCl, 0.1M Hepes pH 7.5). For crystallisation of the mutant complex, the protein was concentrated to 4 mg/ml solution (in 20 mM Tris-HCl pH 7.5, 50 mM NaCl), 5 mM inhibitor was then added to this solution and the complex was incubated on ice for 30 minutes prior to setting up crystallisation trials. The drops contained a 1:1 mixture of complex solution and reservoir buffer (2.6-2.8M NaCl, 0.1M Hepes pH 9.0). The wild type and mutant crystals are isomorphous and belong to space group P4₁2₁2 with unit cell dimensions a=b=56 Å and c=263 Å, and contain two molecules per crystal asymmetric unit. The term “space group” refers to the arrangement of symmetry elements within a unit cell.

### EXAMPLE 4

Determination of the Three dimensional Structure of the Catalytic Domain of MMP9

Three dimensional diffraction data extending to a resolution of 2.3 Å (2.1 Å for the mutant) were collected at the Synchrotron Radiation Source, Daresbury, UK (station 9.6; wavelength 0.87 Å). Data were collected with an ADSC CCD detector at room temperature from more than one crystal. These images were evaluated using the MOSFLM4 software package. The CCP4 suite of programs was used for the data reduction and subsequent data handling. 77,726 observations of 17,375 unique reflections were merged with an R-factor of 11.7% (mutant data: 50,609 observations of 18,841 unique reflections were merged with an R-factor of 14.7%). The wild type structure was solved by molecular replacement, using the program AMoRe and a model based on PDB entry 1HFS, the structure of uninhibited stromelysin. The current model was constructed by interactive model building using the program Quanta98 and refined using X-PLOR. In the early stages of model building, real-space averaging using RAVE significantly improved the quality of electron density maps. The current model of the wild type catalytic domain was inspected against electron density maps and comprises 159 (156, molecule 2) out of 163 residues; 2 Zn2+ and 5 Ca2+ ions; 1 inhibitor and 54 water molecules per subunit. The omitted residues are at the N-terminus. Residue Arg424 has only weak electron density beyond the Cδ atom (Cβ in the second molecule in the crystal asymmetric unit). The R-factor of the refined model is 22.1% (Rfree=26.9%) and the stereo-chemistry is reasonable (r.m.s. deviation of bonds=0.011, angles=1.5). The mutant structure was determined by molecular replacement using the refined wild type structure as trial model. The current model of the mutant was constructed by interactive model building using the program Quanta98 and refined using CNX. The current model of the mutated catalytic domain was inspected against electron density maps and comprises 159 (155, subunit 2) out of 163 residues; 2 Zn2+ and 5 Ca2+ ions; 1 inhibitor and 90 water molecules per subunit. The omitted residues are at the N-terminus. Residue Arg424 has only weak electron density beyond the Cδ atom (Cβ in subunit 2). The R-factor of the refined model is 21.3% (Rfree=25.8%) and the stereo-chemistry is reasonable (r.m.s. deviation of bonds=0.006, angles=1.1).). The R-factor of the refined mutant model is 21.3% (Rfree=25.8%) and the stereo-chemistry is reasonable (r.m.s. deviation of bonds=0.006, angles=1.1).

### REFERENCES

The contents of all documents cited above and below are incorporated herein by reference.

Alexander, C. M. & Werb, Z, In: Hay, E. D. ed. “Cell Biology of the Extracellular Matrix” New York, Plenum Press, 255-302 (1991).

Bacon, D. & Anderson, W. F. Journal of Molecular Graphics 6, 219-220 (1998).

Becker, J. W. et al. Protein Sci. 4, 1966-1976 (1995).

Beckett R. P. & Whittaker M., Exp. Opin. Ther. Patents, 8(3), 259-282 (1998).

Birkedal-Hansen, H. Curr. Opin. Cell Biol. 7, 728-735 (1995).

Bode W. et al. Embo J. 13, 1263-1269 (1994).

Borkakoti, N. et al. Nature Struct. Biology 1, 106-110 (1994).

Brown, D. M. et al, Circulation 91, 2125-2131 (1995).

Browner, M. F., Smith, W. W. & Casteihano, A. L. Biochemistry 34, 6602-6610 (1995).

Chen, J. M. et al. J. Am. Chem. Soc. 122, 9648-9654 (2000).

Collier. I. E. et al J. Biol. Chem. 263, 6579 (1988).

Dhanaraj, V. et al. Structure 4, 375-386 (1996).

Dhanaraj, V. et al. Croatica Chemica Acta 72, 575-591 (1999).

Dollery, C. M. et al, Circulation Research 77, 863-868 (1995).

Esnouf, R. M. Journal of Molecular Graphics 15, 133-138 (1997).

Finzel, B. C. et al. Protein Sci. 7, 2118-2126 (1998).

Galis, Z. S. et al, J. Clin. Invest. 94, 2493-2503 (1994).

Gomis-Rüth, F. X. et al. Nature 389, 77-81 (1997).

Gooley, P. R. et al. Nature Struct. Biology 1, 111-118 (1994).

Grams, F. et al. Biochemistry 34, 14012-14020 (1995).

Grams, F. et al. Eur. J. Biochem. 228, 830-841 (1995a).

Henney, A. M. et al, Proc. Natl. Acad. Sci. USA 88, 8154-8158 (1991).

Li, J. et al. Structure 3, 541-549 (1995).

Liotta et al Biochemistry 20, 100 (1981).

Lovejoy, B., Hassell, A. M., Luther, M. A., Weigl, D. & Jordan, S. R. Biochemistry 33, 8207-8217 (1994a).

Lovejoy, B. et al. Science 263, 375-377 (1994).

Lovejoy, B. et al. Nature Struct. Biology 6, 217-221 (1999).

Merritt, E. A. & Murphy, M. E. P. Acta Crystallogr. D50, 869-873 (1994).

Morgunova, E. et al. Science 284, 1667-1670 (1999).

Moy, F. J. et al. J. Mol. Biol. 302, 671-689 (2000).

Moy, F. J. et al. J. Biomol. NMR 10, 9-19 (1997).

Moy, F. J. et al. Biochemistry 37, 1495-1504 (1998).

Moy, F. J. et al. Biochemistry 38, 7085-7096 (1999).

Murphy, G & Reynolds, J. In: Royce, P. M. & Steinman, B. eds. “Connective Tissue and its Heritable Disorders. New York, Wiley-Liss Inc., 287-316 (1993).

Nicholls, A., Sharp, K. A. & Honig, B. Proteins 11, 281-296 (1991).

Pavlovsky, A. G. et al. Protein Sci. 8, 1455-1462 (1999).

Reinemer, P. et al. Febs Lett 338, 227-233 (1994).

Rhode, L. E. et al Circulation 99, 3063-3070 (1999).

Shipley, J. M. et al. J. Biol. Chem. 271, 4335-4341 (1996).

Skiles et al, Current Medicinal Chemistry, 8, 425-474 (2001).

Spurlino, J. C. et al. Proteins: Struct. Funct. Genet. 19, 98-109 (1994).

Stams, T. et al. Nature Struct. Biology 1, 119-123 (1994).

Steele, D. L. et al. Protein Engineering 13, 397405 (2000).

Stockman, B. J. et al. Protein Sci. 7, 2281-2286 (1998).

Van Doren, S. R et al. Protein Sci. 4, 2487-2498 (1995).

Vu, T. H. & Werb Z. in Matrix Metalloproteinases edited by W. C. Parks & R. P. Mecham, Academic Press, ISBN 0-12-545090-7, 115-148 (1998).

Whittaker, M. et al, Chem. Rev. 99, 2735-2776 (1999).

Wilhelm et al J. Biol. Chem. 264, 17213 (1989); published erratum in J. Biol. Chem. 265, 22570. (1990).

Zhang, X. et al. J. Mol. Biol. 301, 513-524 (2000).

TABLE 2
Three dimensional atomic coordinates of the wild type MMP9
construct in complex with the reverse hydroxamate compound of Formula I.

REMARK coordinates from restrained individual B-factor refinement
REMARK refinement resolution: 100 - 2.3 A
REMARK starting r= 0.2096 free_r= 0.2397
REMARK final r= 0.2074 free_r= 0.2408
REMARK B rmsd for bonded mainchain atoms= 1.707 target= 1.5
REMARK B rmsd for bonded sidechain atoms= 2.673 target= 2.0
REMARK B rmsd for angle mainchain atoms= 2.714 target= 2.0
REMARK B rmsd for angle sidechain atoms= 3.856 target= 2.5
REMARK wa= 2.19316
REMARK rweight=5.978512E-02
REMARK target= mlf steps= 30
REMARK sg= P4(1)2(1)2 a= 56.005 b= 56.005 c= 262.655
alpha= 90 beta= 90 gamma= 9
REMARK parameter file 1 : MSI_CNX_TOPPAR:protein_rep.param
REMARK parameter file 2 : MSI_CNX_TOPPAR:ion.param
REMARK parameter file 3 : MSI_CNX_TOPPAR:water_rep.param
REMARK parameter file 4 : fra.par
REMARK molecular structure file: mmp9.mtf
REMARK input coordinates: anneal_restrain_1.pdb
REMARK reflection file= mmp9_rf.hkl
REMARK ncs= restrain ncs file= ncs_restrain.def
REMARK B-correction resolution: 6.0 - 2.3
REMARK initial B-factor correction applied to fobs:
REMARK B11= −4.644 B22= −4.644 B33= 9.288
REMARK B12= 0.000 B13= 0.000 B23= 0.000
REMARK B-factor correction applied to coordinate array B: 0.533
REMARK bulk solvent: (Mask) density level= 0.372729 e/A\hat{ }3, B-factor 48.8354 A\hat{ }2
REMARK reflections with |Fobs|/sigma_F < 0.0 rejected
REMARK reflections with |Fobs|>1000 * rms(Fobs) rejected
REMARK theoretical total number of refl. in resol. range: 19670 (100.0%)
REMARK number of unobserved reflections (no entry or |F|=0): 2295 (11.7%)
REMARK number of reflections rejected: 0(0.0%)
REMARK total number of reflections used: 17375 (88.3%)
REMARK number of reflections in working set: 16691 (84.9%)
REMARK number of reflections in test set: 684 (3.5%)
REMARK FILENAME=“bindividual7.pdb”
REMARK DATE:Nov-30-2000 13:59:53 created by user: sian
REMARK Written by CNX VERSION:2000
CRYST1 56.005 56.005 262.655 90.00 90.00 90.00 P 41 21 2
SCALE1 0.017856 0.000000 0.000000 0.00000
SCALE2 0.000000 0.017856 0.000000 0.00000
SCALE3 0.000000 0.000000 0.003807 0.00000

| | | | | | | | | | | | |
|---|---|---|---|---|---|---|---|---|---|---|---|
| ATOM | 1 | CB | PHE A | 110 | 68.144 | 27.293 | 107.042 | 1.00 | 76.67 | A | C |
| ATOM | 2 | CG | PHE A | 110 | 69.059 | 26.231 | 107.577 | 1.00 | 76.83 | A | C |
| ATOM | 3 | CD1 | PHE A | 110 | 70.433 | 26.309 | 107.372 | 1.00 | 76.63 | A | C |
| ATOM | 4 | CD2 | PHE A | 110 | 68.547 | 25.145 | 108.276 | 1.00 | 76.62 | A | C |
| ATOM | 5 | CE1 | PHE A | 110 | 71.282 | 25.322 | 107.853 | 1.00 | 75.40 | A | C |
| ATOM | 6 | CB2 | PHE A | 110 | 69.390 | 24.152 | 108.763 | 1.00 | 76.11 | A | C |
| ATOM | 7 | CZ | PHE A | 110 | 70.760 | 24.241 | 108.550 | 1.00 | 75.74 | A | C |
| ATOM | 8 | C | PHE A | 110 | 67.022 | 28.261 | 105.029 | 1.00 | 76.18 | A | C |
| ATOM | 9 | O | PHE A | 110 | 65.965 | 28.280 | 105.664 | 1.00 | 76.86 | A | O |
| ATOM | 10 | N | PHE A | 110 | 69.510 | 28.084 | 105.133 | 1.00 | 76.75 | A | N |
| ATOM | 11 | CA | PHE A | 110 | 68.217 | 27.455 | 105.522 | 1.00 | 76.45 | A | C |
| ATOM | 12 | N | GLU A | 111 | 67.204 | 28.928 | 103.893 | 1.00 | 74.63 | A | N |
| ATOM | 13 | CA | GLU A | 111 | 66.148 | 29.724 | 103.279 | 1.00 | 73.01 | A | C |
| ATOM | 14 | CB | GLU A | 111 | 66.490 | 31.215 | 103.335 | 1.00 | 75.82 | A | C |
| ATOM | 15 | CG | GLU A | 111 | 66.819 | 31.745 | 104.726 | 1.00 | 80.41 | A | C |
| ATOM | 16 | CD | GLU A | 111 | 65.689 | 31.554 | 105.725 | 1.00 | 83.58 | A | C |
| ATOM | 17 | OE1 | GLU A | 111 | 64.561 | 32.022 | 105.452 | 1.00 | 85.28 | A | O |
| ATOM | 18 | OE2 | GLU A | 111 | 65.934 | 30.941 | 106.789 | 1.00 | 84.02 | A | 0 |
| ATOM | 19 | C | GLU A | 111 | 66.025 | 29.282 | 101.826 | 1.00 | 69.95 | A | C |
| ATOM | 20 | O | GLU A | 111 | 67.025 | 29.180 | 101.111 | 1.00 | 70.43 | A | 0 |
| ATOM | 21 | N | GLY A | 112 | 64.801 | 29.009 | 101.394 | 1.00 | 66.03 | A | N |
| ATOM | 22 | CA | GLY A | 112 | 64.587 | 28.577 | 100.027 | 1.00 | 60.34 | A | C |
| ATOM | 23 | C | GLY A | 112 | 63.299 | 29.143 | 99.479 | 1.00 | 56.89 | A | C |
| ATOM | 24 | O | GLY A | 112 | 62.515 | 29.744 | 100.213 | 1.00 | 57.97 | A | 0 |
| ATOM | 25 | N | ASP A | 113 | 63.077 | 28.955 | 98.185 | 1.00 | 53.94 | A | N |
| ATOM | 26 | CA | ASP A | 113 | 61.866 | 29.459 | 97.553 | 1.00 | 50.40 | A | C |
| ATOM | 27 | CB | ASP A | 113 | 62.209 | 30.556 | 96.543 | 1.00 | 52.58 | A | C |
| ATOM | 28 | CG | ASP A | 113 | 62.790 | 31.792 | 97.202 | 1.00 | 53.44 | A | C |
| ATOM | 29 | OD1 | ASP A | 113 | 62.092 | 32.405 | 98.039 | 1.00 | 53.76 | A | 0 |
| ATOM | 30 | OD2 | ASP A | 113 | 63.944 | 32.146 | 96.885 | 1.00 | 53.72 | A | 0 |
| ATOM | 31 | C | ASP A | 113 | 61.098 | 28.345 | 96.867 | 1.00 | 46.20 | A | C |
| ATOM | 32 | O | ASP A | 113 | 60.589 | 28.519 | 95.763 | 1.00 | 46.06 | A | 0 |
| ATOM | 33 | N | LEU A | 114 | 61.024 | 27.198 | 97.535 | 1.00 | 40.51 | A | N |
| ATOM | 34 | CA | LEU A | 114 | 60.309 | 26.043 | 97.018 | 1.00 | 35.29 | A | C |
| ATOM | 35 | CB | LEU A | 114 | 61.265 | 24.870 | 96.812 | 1.00 | 35.18 | A | C |
| ATOM | 36 | CG | LEU A | 114 | 62.424 | 25.022 | 95.829 | 1.00 | 35.74 | A | C |
| ATOM | 37 | CD1 | LEU A | 114 | 63.155 | 23.689 | 95.726 | 1.00 | 34.64 | A | C |
| ATOM | 38 | CD2 | LEU A | 114 | 61.906 | 25.455 | 94.463 | 1.00 | 31.83 | A | C |
| ATOM | 39 | C | LEU A | 114 | 59.229 | 25.624 | 98.003 | 1.00 | 33.60 | A | C |
| ATOM | 40 | O | LEU A | 114 | 59.363 | 25.842 | 99.205 | 1.00 | 33.68 | A | 0 |
| ATOM | 41 | N | LYS A | 115 | 58.151 | 25.040 | 97.489 | 1.00 | 30.09 | A | N |
| ATOM | 42 | CA | LYS A | 115 | 57.066 | 24.556 | 98.331 | 1.00 | 28.79 | A | C |
| ATOM | 43 | CB | LYS A | 115 | 55.782 | 25.350 | 98.091 | 1.00 | 27.23 | A | C |
| ATOM | 44 | CG | LYS A | 115 | 55.136 | 25.113 | 96.739 | 1.00 | 30.27 | A | C |
| ATOM | 45 | CD | LYS A | 115 | 53.726 | 25.671 | 96.711 | 1.00 | 31.99 | A | C |
| ATOM | 46 | CE | LYS A | 115 | 53.015 | 25.309 | 95.420 | 1.00 | 33.32 | A | C |
| ATOM | 47 | NZ | LYS A | 115 | 51.602 | 25.772 | 95.434 | 1.00 | 31.74 | A | N |
| ATOM | 48 | C | LYS A | 115 | 56.854 | 23.099 | 97.946 | 1.00 | 27.74 | A | C |
| ATOM | 49 | o | LYS A | 115 | 57.490 | 22.610 | 97.017 | 1.00 | 26.52 | A | O |
| ATOM | 50 | N | TRP A | 116 | 55.984 | 22.398 | 98.660 | 1.00 | 26.86 | A | N |
| ATOM | 51 | CA | TRP A | 116 | 55.725 | 21.005 | 98.327 | 1.00 | 26.50 | A | C |
| ATOM | 52 | CB | TRP A | 116 | 55.139 | 20.242 | 99.525 | 1.00 | 24.26 | A | C |
| ATOM | 53 | CG | TRP A | 116 | 56.071 | 20.142 | 100.708 | 1.00 | 26.96 | A | C |
| ATOM | 54 | CD2 | TRP A | 116 | 57.243 | 19.321 | 100.810 | 1.00 | 26.47 | A | C |
| ATOM | 55 | CE2 | TRP A | 116 | 57.806 | 19.554 | 102.087 | 1.00 | 25.23 | A | C |
| ATOM | 56 | CE3 | TRP A | 116 | 57.873 | 18.414 | 99.946 | 1.00 | 25.47 | A | C |
| ATOM | 57 | CD1 | TRP A | 116 | 55.977 | 20.821 | 101.894 | 1.00 | 23.47 | A | C |
| ATOM | 58 | NE1 | TRP A | 116 | 57.014 | 20.472 | 102.724 | 1.00 | 22.33 | A | N |
| ATOM | 59 | CZ2 | TRP A | 116 | 58.972 | 18.910 | 102.521 | 1.00 | 24.08 | A | C |
| ATOM | 60 | CZ3 | TRP A | 116 | 59.037 | 17.774 | 100.378 | 1.00 | 24.64 | A | C |
| ATOM | 61 | CH2 | TRP A | 116 | 59.572 | 18.027 | 101.654 | 1.00 | 25.34 | A | C |
| ATOM | 62 | C | TRP A | 116 | 54.723 | 20.991 | 97.184 | 1.00 | 27.50 | A | C |
| ATOM | 63 | O | TRP A | 116 | 53.828 | 21.836 | 97.125 | 1.00 | 25.79 | A | O |
| ATOM | 64 | N | HIS A | 117 | 54.877 | 20.039 | 96.271 | 1.00 | 27.87 | A | N |
| ATOM | 65 | CA | HIS A | 117 | 53.950 | 19.932 | 95.158 | 1.00 | 29.48 | A | C |
| ATOM | 66 | CB | HIS A | 117 | 54.692 | 19.528 | 93.885 | 1.00 | 25.71 | A | C |
| ATOM | 67 | CG | HIS A | 117 | 55.384 | 20.674 | 93.214 | 1.00 | 27.00 | A | C |
| ATOM | 68 | CD2 | HIS A | 117 | 55.394 | 21.998 | 93.501 | 1.00 | 27.93 | A | C |
| ATOM | 69 | ND1 | HIS A | 117 | 56.184 | 20.520 | 92.103 | 1.00 | 28.15 | A | N |
| ATOM | 70 | CE1 | HIS A | 117 | 56.659 | 21.697 | 91.736 | 1.00 | 25.56 | A | C |
| ATOM | 71 | NE2 | HIS A | 117 | 56.195 | 22.611 | 92.568 | 1.00 | 25.76 | A | N |
| ATOM | 72 | C | HIS A | 117 | 52.794 | 18.982 | 95.465 | 1.00 | 31.31 | A | C |
| ATOM | 73 | O | HIS A | 117 | 51.849 | 18.873 | 94.687 | 1.00 | 36.62 | A | O |
| ATOM | 74 | N | HIS A | 118 | 52.868 | 18.292 | 96.600 | 1.00 | 28.91 | A | N |
| ATOM | 75 | CA | HIS A | 118 | 51.782 | 17.412 | 97.007 | 1.00 | 27.75 | A | C |
| ATOM | 76 | CB | HIS A | 118 | 52.257 | 15.966 | 97.160 | 1.00 | 26.46 | A | C |
| ATOM | 77 | CG | HIS A | 118 | 53.454 | 15.800 | 98.041 | 1.00 | 28.06 | A | C |
| ATOM | 78 | CD2 | HIS A | 118 | 53.587 | 15.230 | 99.262 | 1.00 | 26.27 | A | C |
| ATOM | 79 | ND1 | HIS A | 118 | 54.715 | 16.213 | 97.672 | 1.00 | 27.31 | A | N |
| ATOM | 80 | CE1 | HIS A | 118 | 55.575 | 15.901 | 98.625 | 1.00 | 25.21 | A | C |
| ATOM | 81 | NE2 | HIS A | 118 | 54.916 | 15.304 | 99.600 | 1.00 | 25.44 | A | N |
| ATOM | 82 | C | HIS A | 118 | 51.232 | 17.949 | 98.322 | 1.00 | 28.02 | A | C |
| ATOM | 83 | O | HIS A | 118 | 51.906 | 18.717 | 99.006 | 1.00 | 30.48 | A | O |
| ATOM | 84 | N | HIS A | 119 | 50.008 | 17.566 | 98.670 | 1.00 | 28.21 | A | N |
| ATOM | 85 | CA | HIS A | 119 | 49.394 | 18.049 | 99.900 | 1.00 | 28.31 | A | C |
| ATOM | 86 | CB | HIS A | 119 | 48.001 | 18.597 | 99.607 | 1.00 | 27.88 | A | C |
| ATOM | 87 | CG | HIS A | 119 | 48.015 | 19.881 | 98.843 | 1.00 | 30.03 | A | C |
| ATOM | 88 | CD2 | HIS A | 119 | 49.045 | 20.642 | 98.402 | 1.00 | 28.56 | A | C |
| ATOM | 89 | ND1 | HIS A | 119 | 46.862 | 20.530 | 98.452 | 1.00 | 32.34 | A | N |
| ATOM | 90 | CE1 | HIS A | 119 | 47.182 | 21.636 | 97.804 | 1.00 | 30.91 | A | C |
| ATOM | 91 | NE2 | HIS A | 119 | 48.500 | 21.728 | 97.760 | 1.00 | 30.11 | A | N |
| ATOM | 92 | C | HIS A | 119 | 49.318 | 17.040 | 101.034 | 1.00 | 28.11 | A | C |
| ATOM | 93 | O | HIS A | 119 | 49.120 | 17.424 | 102.184 | 1.00 | 28.79 | A | O |
| ATOM | 94 | N | ASN A | 120 | 49.447 | 15.757 | 100.715 | 1.00 | 28.28. | A | N |
| ATOM | 95 | CA | ASN A | 120 | 49.418 | 14.724 | 101.742 | 1.00 | 30.67 | A | C |
| ATOM | 96 | CE | ASN A | 120 | 48.753 | 13.442 | 101.206 | 1.00 | 35.59 | A | C |
| ATOM | 97 | CG | ASN A | 120 | 49.339 | 12.970 | 99.874 | 1.00 | 40.97 | A | C |
| ATOM | 98 | OD1 | ASN A | 120 | 49.628 | 13.776 | 98.982 | 1.00 | 42.91 | A | O |
| ATOM | 99 | ND2 | ASN A | 120 | 49.494 | 11.652 | 99.728 | 1.00 | 42.68 | A | N |
| ATOM | 100 | C | ASN A | 120 | 50.873 | 14.489 | 102.141 | 1.00 | 29.30 | A | C |
| ATOM | 101 | O | ASN A | 120 | 51.564 | 13.634 | 101.585 | 1.00 | 29.67 | A | O |
| ATOM | 102 | N | ILE A | 121 | 51.330 | 15.287 | 103.103 | 1.00 | 26.98 | A | N |
| ATOM | 103 | CA | ILE A | 121 | 52.710 | 15.233 | 103.577 | 1.00 | 26.04 | A | C |
| ATOM | 104 | CE | ILE A | 121 | 53.138 | 16.617 | 104.109 | 1.00 | 26.73 | A | C |
| ATOM | 105 | CG2 | ILE A | 121 | 54.574 | 16.568 | 104.626 | 1.00 | 26.58 | A | C |
| ATOM | 106 | CG1 | ILE A | 121 | 52.997 | 17.650 | 102.987 | 1.00 | 29.00 | A | C |
| ATOM | 107 | CD1 | ILE A | 121 | 53.451 | 19.037 | 103.357 | 1.00 | 32.81 | A | C |
| ATOM | 108 | C | ILE A | 121 | 52.945 | 14.179 | 104.650 | 1.00 | 23.95 | A | C |
| ATOM | 109 | O | ILE A | 121 | 52.081 | 13.928 | 105.484 | 1.00 | 24.37 | A | O |
| ATOM | 110 | N | THR A | 122 | 54.119 | 13.560 | 104.621 | 1.00 | 20.08 | A | N |
| ATOM | 111 | CA | THR A | 122 | 54.441 | 12.534 | 105.598 | 1.00 | 21.17 | A | C |
| ATOM | 112 | CB | THR A | 122 | 54.763 | 11.172 | 104.914 | 1.00 | 22.42 | A | C |
| ATOM | 113 | OG1 | THR A | 122 | 55.948 | 11.294 | 104.115 | 1.00 | 20.53 | A | O |
| ATOM | 114 | CG2 | THR A | 122 | 53.592 | 10.722 | 104.042 | 1.00 | 21.00 | A | C |
| ATOM | 115 | C | THR A | 122 | 55.624 | 12.925 | 106.481 | 1.00 | 20.68 | A | C |
| ATOM | 116 | O | THR A | 122 | 56.502 | 13.706 | 106.081 | 1.00 | 18.28 | A | O |
| ATOM | 117 | N | TYR A | 123 | 55.640 | 12.382 | 107.693 | 1.00 | 18.69 | A | N |
| ATOM | 118 | CA | TYR A | 123 | 56.728 | 12.671 | 108.612 | 1.00 | 17.02 | A | C |
| ATOM | 119 | CB | TYR A | 123 | 56.374 | 13.828 | 109.561 | 1.00 | 12.64 | A | C |
| ATOM | 120 | CG | TYR A | 123 | 55.291 | 13.533 | 110.579 | 1.00 | 14.99 | A | C |
| ATOM | 121 | CD1 | TYR A | 123 | 53.949 | 13.737 | 110.277 | 1.00 | 15.83 | A | C |
| ATOM | 122 | CE1 | TYR A | 123 | 52.953 | 13.517 | 111.228 | 1.00 | 14.44 | A | C |
| ATOM | 123 | CD2 | TYR A | 123 | 55.613 | 13.088 | 111.867 | 1.00 | 15.15 | A | C |
| ATOM | 124 | CE2 | TYR A | 123 | 54.623 | 12.866 | 112.821 | 1.00 | 13.15 | A | C |
| ATOM | 125 | CZ | TYR A | 123 | 53.296 | 13.087 | 112.495 | 1.00 | 14.54 | A | C |
| ATOM | 126 | OH | TYR A | 123 | 52.305 | 12.912 | 113.439 | 1.00 | 17.15. | A | O |
| ATOM | 127 | C | TYR A | 123 | 57.109 | 11.446 | 109.419 | 1.00 | 15.63 | A | C |
| ATOM | 128 | O | TYR A | 123 | 56.277 | 10.594 | 109.735 | 1.00 | 14.88 | A | O |
| ATOM | 129 | N | TRP A | 124 | 58.387 | 11.380 | 109.745 | 1.00 | 14.30 | A | N |
| ATOM | 130 | CA | TRP A | 124 | 58.933 | 10.288 | 110.504 | 1.00 | 15.16 | A | C |
| ATOM | 131 | CB | TRP A | 124 | 59.876 | 9.486 | 109.603 | 1.00 | 17.22 | A | C |
| ATOM | 132 | CG | TRP A | 124 | 60.649 | 8.390 | 110.274 | 1.00 | 14.19 | A | C |
| ATOM | 133 | CD2 | TRP A | 124 | 62.005 | 8.019 | 110.004 | 1.00 | 17.22 | A | C |
| ATOM | 134 | CE2 | TRP A | 124 | 62.299 | 6.892 | 110.807 | 1.00 | 18.18 | A | C |
| ATOM | 135 | CE3 | TRP A | 124 | 63.002 | 8.525 | 109.157 | 1.00 | 17.93 | A | C |
| ATOM | 136 | CD1 | TRP A | 124 | 60.186 | 7.506 | 111.204 | 1.00 | 15.79 | A | C |
| ATOM | 137 | NE1 | TRP A | 124 | 61.172 | 6.599 | 111.530 | 1.00 | 17.60 | A | N |
| ATOM | 138 | CZ2 | TRP A | 124 | 63.548 | 6.262 | 110.788 | 1.00 | 17.69 | A | C |
| ATOM | 139 | CZ3 | TRP A | 124 | 64.244 | 7.899 | 109.139 | 1.00 | 22.22 | A | C |
| ATOM | 140 | CH2 | TRP A | 124 | 64.505 | 6.776 | 109.951 | 1.00 | 19.94 | A | C |
| ATOM | 141 | C | TRP A | 124 | 59.679 | 10.321 | 111.720 | 1.00 | 15.01 | A | C |
| ATOM | 142 | O | TRP A | 124 | 60.575 | 11.645 | 111.590 | 1.00 | 15.43 | A | O |
| ATOM | 143 | N | ILE A | 125 | 59.283 | 10.365 | 112.903 | 1.00 | 13.81 | A | N |
| ATOM | 144 | CA | ILE A | 125 | 59.960 | 10.754 | 114.127 | 1.00 | 14.20 | A | C |
| ATOM | 145 | CB | ILE A | 125 | 59.009 | 10.697 | 115.341 | 1.00 | 14.44 | A | C |
| ATOM | 146 | CG2 | ILE A | 125 | 59.758 | 11.087 | 116.609 | 1.00 | 10.36 | A | C |
| ATOM | 147 | CG1 | ILE A | 125 | 57.833 | 11.649 | 115.110 | 1.00 | 14.53 | A | C |
| ATOM | 148 | CD1 | ILE A | 125 | 56.721 | 11.500 | 116.106 | 1.00 | 16.01 | A | C |
| ATOM | 149 | C | ILE A | 125 | 61.033 | 9.685 | 114.249 | 1.00 | 14.73 | A | C |
| ATOM | 150 | O | ILE A | 125 | 60.763 | 8.564 | 114.660 | 1.00 | 17.21 | A | O |
| ATOM | 151 | N | GLN A | 126 | 62.259 | 10.036 | 113.863 | 1.00 | 15.17 | A | N |
| ATOM | 152 | CA | GLN A | 126 | 63.374 | 9.102 | 113.877 | 1.00 | 17.19 | A | C |
| ATOM | 153 | CB | GLN A | 126 | 64.540 | 9.679 | 113.065 | 1.00 | 15.71 | A | C |
| ATOM | 154 | CG | GLN A | 126 | 65.810 | 8.849 | 113.134 | 1.00 | 19.08 | A | C |
| ATOM | 155 | CD | GLN A | 126 | 67.006 | 9.537 | 112.492 | 1.00 | 21.76 | A | C |
| ATOM | 156 | OE1 | GLN A | 126 | 63.141 | 9.434 | 112.978 | 1.00 | 21.48 | A | O |
| ATOM | 157 | NE2 | GLN A | 126 | 66.762 | 10.235 | 111.395 | 1.00 | 21.97 | A | N |
| ATOM | 158 | C | GLN A | 126 | 63.858 | 3.702 | 115.270 | 1.00 | 18.20 | A | C |
| ATOM | 159 | O | GLN A | 126 | 64.062 | 7.519 | 115.536 | 1.00 | 18.29 | A | O |
| ATOM | 160 | N | ASN A | 127 | 64.066 | 9.681 | 116.145 | 1.00 | 19.88 | A | N |
| ATOM | 161 | CA | ASN A | 127 | 64.515 | 9.397 | 117.504 | 1.00 | 18.22 | A | C |
| ATOM | 162 | CB | ASN A | 127 | 66.042 | 9.423 | 117.594 | 1.00 | 19.01 | A | C |
| ATOM | 163 | CG | ASN A | 127 | 66.631 | 10.770 | 117.242 | 1.00 | 21.39 | A | C |
| ATOM | 164 | OD1 | ASN A | 127 | 65.957 | 11.798 | 117.310 | 1.00 | 25.90 | A | O |
| ATOM | 165 | ND2 | ASN A | 127 | 67.909 | 10.774 | 116.880 | 1.00 | 20.17 | A | N |
| ATOM | 166 | C | ASN A | 127 | 63.900 | 10.336 | 118.483 | 1.00 | 19.18 | A | C |
| ATOM | 167 | O | ASN A | 127 | 63.238 | 11.341 | 118.068 | 1.00 | 18.72 | A | O |
| ATOM | 168 | N | TYR A | 128 | 64.128 | 10.163 | 119.779 | 1.00 | 17.90 | A | N |
| ATOM | 169 | CA | TYR A | 128 | 63.539 | 11.006 | 120.822 | 1.00 | 15.61 | A | C |
| ATOM | 170 | CB | TYR A | 128 | 62.592 | 10.166 | 121.693 | 1.00 | 15.35 | A | C |
| ATOM | 171 | CG | TYR A | 128 | 61.389 | 9.623 | 120.957 | 1.00 | 14.14 | A | C |
| ATOM | 172 | CD1 | TYR A | 128 | 61.476 | 8.467 | 120.178 | 1.00 | 121.53 | A | C |
| ATOM | 173 | CE1 | TYR A | 128 | 60.374 | 8.000 | 119.469 | 1.00 | 12.28 | A | C |
| ATOM | 174 | CD2 | TYR A | 128 | 60.172 | 10.293 | 121.008 | 1.00 | 12.14 | A | C |
| ATOM | 175 | CE2 | TYR A | 128 | 59.078 | 9.839 | 120.306 | 1.00 | 14.27 | A | C |
| ATOM | 176 | CZ | TYR A | 128 | 59.182 | 8.698 | 119.539 | 1.00 | 13.46 | A | C |
| ATOM | 177 | OH | TYR A | 128 | 58.084 | 8.292 | 118.837 | 1.00 | 12.04 | A | O |
| ATOM | 178 | C | TYR A | 128 | 64.501 | 11.716 | 121.746 | 1.00 | 14.76 | A | C |
| ATOM | 179 | O | TYR A | 128 | 65.681 | 11.396 | 121.808 | 1.00 | 15.87 | A | O |
| ATOM | 180 | N | SER A | 129 | 63.969 | 12.693 | 122.471 | 1.00 | 17.19 | A | N |
| ATOM | 181 | CA | SER A | 129 | 64.742 | 13.430 | 123.460 | 1.00 | 19.66 | A | C |
| ATOM | 182 | CB | SER A | 129 | 64.173 | 14.826 | 123.675 | 1.00 | 17.51 | A | C |
| ATOM | 183 | OG | SER A | 129 | 64.731 | 15.394 | 124.844 | 1.00 | 16.48 | A | O |
| ATOM | 184 | C | SER A | 129 | 64.600 | 12.640 | 124.753 | 1.00 | 22.10 | A | C |
| ATOM | 185 | O | SER A | 129 | 63.646 | 11.882 | 124.920 | 1.00 | 21.65 | A | O |
| ATOM | 186 | N | GLU A | 130 | 65.529 | 12.821 | 125.677 | 1.00 | 24.54 | A | N |
| ATOM | 187 | CA | GLU A | 130 | 65.459 | 12.085 | 126.925 | 1.00 | 28.04 | A | C |
| ATOM | 188 | CB | GLU A | 130 | 66.884 | 11.859 | 127.459 | 1.00 | 31.45 | A | C |
| ATOM | 189 | CG | GLU A | 130 | 67.799 | 11.117 | 126.464 | 1.00 | 36.20 | A | C |
| ATOM | 190 | CD | GLU A | 130 | 69.207 | 10.871 | 126.996 | 1.00 | 37.68 | A | C |
| ATOM | 191 | OE1 | GLU A | 130 | 69.330 | 10.501 | 128.182 | 1.00 | 43.10 | A | O |
| ATOM | 192 | OE2 | GLU A | 130 | 70.189 | 11.031 | 126.237 | 1.00 | 32.89 | A | O |
| ATOM | 193 | C | GLU A | 130 | 64.587 | 12.788 | 127.971 | 1.00 | 27.09 | A | C |
| ATOM | 194 | O | GLU A | 130 | 64.154 | 12.167 | 128.939 | 1.00 | 29.20 | A | O |
| ATOM | 195 | N | ASP A | 131 | 64.301 | 14.069 | 127.751 | 1.00 | 23.80 | A | N |
| ATOM | 196 | CA | ASP A | 131 | 63.519 | 14.870 | 128.693 | 1.00 | 21.55 | A | C |
| ATOM | 197 | CB | ASP A | 131 | 63.657 | 16.355 | 128.367 | 1.00 | 20.53 | A | C |
| ATOM | 198 | CG | ASP A | 131 | 65.084 | 16.774 | 128.149 | 1.00 | 21.96 | A | C |
| ATOM | 199 | OD1 | ASP A | 131 | 65.993 | 16.132 | 128.718 | 1.00 | 23.79 | A | O |
| ATOM | 200 | OD2 | ASP A | 131 | 65.291 | 17.760 | 127.417 | 1.00 | 19.14 | A | O |
| ATOM | 201 | C | ASP A | 131 | 62.029 | 14.589 | 128.821 | 1.00 | 22.60 | A | C |
| ATOM | 202 | O | ASP A | 131 | 61.417 | 14.946 | 129.826 | 1.00 | 22.01 | A | O |
| ATOM | 203 | N | LEU A | 132 | 61.426 | 13.985 | 127.808 | 1.00 | 21.60 | A | N |
| ATOM | 204 | CA | LEU A | 132 | 59.996 | 13.736 | 127.862 | 1.00 | 17.46 | A | C |
| ATOM | 205 | CB | LEU A | 132 | 59.274 | 14.744 | 126.964 | 1.00 | 18.77 | A | C |
| ATOM | 206 | CG | LEU A | 132 | 59.410 | 16.228 | 127.329 | 1.00 | 16.38 | A | C |
| ATOM | 207 | CD1 | LEU A | 132 | 58.952 | 17.103 | 126.185 | 1.00 | 16.14 | A | C |
| ATOM | 208 | CD2 | LEU A | 132 | 58.584 | 16.513 | 128.574 | 1.00 | 17.73 | A | C |
| ATOM | 209 | C | LEU A | 132 | 59.625 | 12.322 | 127.447 | 1.00 | 17.91 | A | C |
| ATOM | 210 | O | LEU A | 132 | 60.350 | 11.668 | 126.703 | 1.00 | 20.57 | A | O |
| ATOM | 211 | N | PRO A | 133 | 58.493 | 11.818 | 127.952 | 1.00 | 18.54 | A | N |
| ATOM | 212 | CD | PRO A | 133 | 57.590 | 12.397 | 128.962 | 1.00 | 16.46 | A | C |
| ATOM | 213 | CA | PRO A | 133 | 58.070 | 10.464 | 127.588 | 1.00 | 18.30 | A | C |
| ATOM | 214 | CB | PRO A | 133 | 56.828 | 10.250 | 128.448 | 1.00 | 16.44 | A | C |
| ATOM | 215 | CG | PRO A | 133 | 57.029 | 11.174 | 129.599 | 1.00 | 13.96 | A | C |
| ATOM | 216 | C | PRO A | 133 | 57.739 | 10.482 | 126.092 | 1.00 | 18.28 | A | C |
| ATOM | 217 | O | PRO A | 133 | 57.305 | 11.511 | 125.566 | 1.00 | 15.16 | A | O |
| ATOM | 218 | N | ARG A | 134 | 57.931 | 9.358 | 125.412 | 1.00 | 18.49 | A | N |
| ATOM | 219 | CA | ARG A | 134 | 57.661 | 9.313 | 123.978 | 1.00 | 20.66 | A | C |
| ATOM | 220 | CB | ARG A | 134 | 58.084 | 7.961 | 123.411 | 1.00 | 20.98 | A | C |
| ATOM | 221 | CG | ARG A | 134 | 59.563 | 7.723 | 123.628 | 1.00 | 21.85 | A | C |
| ATOM | 222 | CD | ARG A | 134 | 60.064 | 6.476 | 122.953 | 1.00 | 25.93 | A | C |
| ATOM | 223 | NE | ARG A | 134 | 61.506 | 6.346 | 123.137 | 1.00 | 29.18 | A | N |
| ATOM | 224 | CZ | ARG A | 134 | 62.234 | 5.347 | 122.652 | 1.00 | 32.95 | A | C |
| ATOM | 225 | NH1 | ARG A | 134 | 61.649 | 4.384 | 121.949 | 1.00 | 31.93 | A | N |
| ATOM | 226 | NH2 | ARG A | 134 | 63.544 | 5.314 | 122.867 | 1.00 | 31.66 | A | N |
| ATOM | 227 | C | ARG A | 134 | 56.217 | 9.633 | 123.615 | 1.00 | 20.30 | A | C |
| ATOM | 228 | O | ARG A | 134 | 55.961 | 10.226 | 122.575 | 1.00 | 19.91 | A | O |
| ATOM | 229 | N | ALA A | 135 | 55.274 | 9.256 | 124.470 | 1.00 | 19.00 | A | N |
| ATOM | 230 | CA | ALA A | 135 | 53.871 | 9.552 | 124.201 | 1.00 | 20.03 | A | C |
| ATOM | 231 | CB | ALA A | 135 | 52.980 | 8.861 | 125.237 | 1.00 | 18.70 | A | C |
| ATOM | 232 | C | ALA A | 135 | 53.642 | 11.073 | 124.226 | 1.00 | 19.99 | A | C |
| ATOM | 233 | O | ALA A | 135 | 52.814 | 11.601 | 123.489 | 1.00 | 19.37 | A | O |
| ATOM | 234 | N | VAL A | 136 | 54.378 | 11.774 | 125.082 | 1.00 | 18.65 | A | N |
| ATOM | 235 | CA | VAL A | 136 | 54.245 | 13.227 | 125.175 | 1.00 | 18.87 | A | C |
| ATOM | 236 | CB | VAL A | 136 | 54.950 | 13.777 | 126.459 | 1.00 | 18.18 | A | C |
| ATOM | 237 | CG1 | VAL A | 136 | 54.999 | 15.309 | 126.443 | 1.00 | 16.50 | A | C |
| ATOM | 238 | CG2 | VAL A | 136 | 54.204 | 13.303 | 127.688 | 1.00 | 13.02 | A | C |
| ATOM | 239 | C | VAL A | 136 | 54.838 | 13.900 | 123.930 | 1.00 | 17.85 | A | C |
| ATOM | 240 | O | VAL A | 136 | 54.277 | 14.870 | 123.400 | 1.00 | 16.07 | A | O |
| ATOM | 241 | N | ILE A | 137 | 55.969 | 13.374 | 123.469 | 1.00 | 14.93 | A | N |
| ATOM | 242 | CA | ILE A | 137 | 56.630 | 13.916 | 122.295 | 1.00 | 13.94 | A | C |
| ATOM | 243 | CB | ILE A | 137 | 58.035 | 13.292 | 122.121 | 1.00 | 13.55 | A | C |
| ATOM | 244 | CG2 | ILE A | 137 | 58.657 | 13.730 | 120.789 | 1.00 | 10.34 | A | C |
| ATOM | 245 | CG1 | ILE A | 137 | 58.917 | 13.705 | 123.307 | 1.00 | 11.84 | A | C |
| ATOM | 246 | CD1 | ILE A | 137 | 60.329 | 13.165 | 123.274 | 1.00 | 8.78 | A | C |
| ATOM | 247 | C | ILE A | 137 | 55.781 | 13.689 | 121.044 | 1.00 | 13.89 | A | C |
| ATOM | 248 | O | ILE A | 137 | 55.557 | 14.616 | 120.270 | 1.00 | 13.43 | A | O |
| ATOM | 249 | N | ASP A | 138 | 55.298 | 12.465 | 120.850 | 1.00 | 14.79 | A | N |
| ATOM | 250 | CA | ASP A | 138 | 54.469 | 12.168 | 119.685 | 1.00 | 16.05 | A | C |
| ATOM | 251 | CB | ASP A | 138 | 53.891 | 10.748 | 119.764 | 1.00 | 14.50 | A | C |
| ATOM | 252 | CG | ASP A | 138 | 54.946 | 9.662 | 119.593 | 1.00 | 16.77 | A | C |
| ATOM | 253 | OD1 | ASP A | 138 | 56.089 | 9.967 | 119.188 | 1.00 | 18.40 | A | O |
| ATOM | 254 | OD2 | ASP A | 138 | 54.619 | 8.484 | 119.853 | 1.00 | 20.23 | A | O |
| ATOM | 255 | C | ASP A | 138 | 53.317 | 13.170 | 119.602 | 1.00 | 16.70 | A | C |
| ATOM | 256 | O | ASP A | 138 | 53.086 | 13.784 | 118.564 | 1.00 | 19.57 | A | O |
| ATOM | 257 | N | ASP A | 139 | 52.612 | 13.337 | 120.716 | 1.00 | 16.62 | A | N |
| ATOM | 258 | CA | ASP A | 139 | 51.460 | 14.232 | 120.801 | 1.00 | 15.09 | A | C |
| ATOM | 259 | CB | ASP A | 139 | 50.812 | 14.099 | 122.181 | 1.00 | 14.62 | A | C |
| ATOM | 260 | CG | ASP A | 139 | 49.611 | 15.003 | 122.354 | 1.00 | 16.52 | A | C |
| ATOM | 261 | OD1 | ASP A | 139 | 48.584 | 14.769 | 121.692 | 1.00 | 19.96 | A | O |
| ATOM | 262 | OD2 | ASP A | 139 | 49.688 | 15.953 | 123.152 | 1.00 | 12.35 | A | O |
| ATOM | 263 | C | ASP A | 139 | 51.785 | 15.693 | 120.526 | 1.00 | 15.75 | A | C |
| ATOM | 264 | O | ASP A | 139 | 51.004 | 16.396 | 119.894 | 1.00 | 17.52 | A | O |
| ATOM | 265 | N | ALA A | 140 | 52.935 | 16.152 | 121.008 | 1.00 | 16.49 | A | N |
| ATOM | 266 | CA | ALA A | 140 | 53.356 | 17.536 | 120.805 | 1.00 | 14.14 | A | C |
| ATOM | 267 | CB | ALA A | 140 | 54.648 | 17.819 | 121.586 | 1.00 | 10.53 | A | C |
| ATOM | 268 | C | ALA A | 140 | 53.576 | 17.805 | 119.319 | 1.00 | 13.23 | A | C |
| ATOM | 269 | O | ALA A | 140 | 53.156 | 18.838 | 118.797 | 1.00 | 13.50 | A | O |
| ATOM | 270 | N | PHE A | 141 | 54.249 | 16.877 | 118.645 | 1.00 | 12.36 | A | N |
| ATOM | 271 | CA | PHE A | 141 | 54.501 | 17.017 | 117.216 | 1.00 | 15.20 | A | C |
| ATOM | 272 | CB | PHE A | 141 | 55.431 | 15.901 | 116.722 | 1.00 | 13.79 | A | C |
| ATOM | 273 | CG | PHE A | 141 | 56.867 | 16.073 | 117.146 | 1.00 | 11.40 | A | C |
| ATOM | 274 | CD1 | PHE A | 141 | 57.408 | 17.340 | 117.318 | 1.00 | 12.07 | A | C |
| ATOM | 275 | CD2 | PHE A | 141 | 57.685 | 14.973 | 117.341 | 1.00 | 11.50 | A | C |
| ATOM | 276 | CE1 | PHE A | 141 | 58.752 | 17.507 | 117.678 | 1.00 | 15.23 | A | C |
| ATOM | 277 | CE2 | PHE A | 141 | 59.032 | 15.133 | 117.701 | 1.00 | 10.84 | A | C |
| ATOM | 278 | CZ | PHE A | 141 | 59.564 | 16.396 | 117.868 | 1.00 | 9.69 | A | C |
| ATOM | 279 | C | PHE A | 141 | 53.183 | 16.988 | 116.433 | 1.00 | 15.88 | A | C |
| ATOM | 280 | O | PHE A | 141 | 52.980 | 17.783 | 115.516 | 1.00 | 18.45 | A | O |
| ATOM | 281 | N | ALA A | 142 | 52.289 | 16.080 | 116.815 | 1.00 | 15.46 | A | N |
| ATOM | 282 | CA | ALA A | 142 | 50.997 | 15.940 | 116.156 | 1.00 | 16.51 | A | C |
| ATOM | 283 | CB | ALA A | 142 | 50.235 | 14.769 | 116.751 | 1.00 | 16.42 | A | C |
| ATOM | 284 | C | ALA A | 142 | 50.175 | 17.217 | 116.281 | 1.00 | 17.88 | A | C |
| ATOM | 285 | O | ALA A | 142 | 49.519 | 17.642 | 115.327 | 1.00 | 14.95 | A | O |
| ATOM | 286 | N | ARG A | 143 | 50.207 | 17.826 | 117.464 | 1.00 | 17.58 | A | N |
| ATOM | 287 | CA | ARG A | 143 | 49.465 | 19.055 | 117.687 | 1.00 | 16.25 | A | C |
| ATOM | 288 | CB | ARG A | 143 | 49.349 | 19.353 | 119.191 | 1.00 | 13.94 A | C | |
| ATOM | 289 | CC | ARG A | 143 | 48.421 | 18.387 | 119.930 | 1.00 | 13.87 | A | C |
| ATOM | 290 | CD | ARG A | 143 | 48.231 | 18.747 | 121.404 | 1.00 | 13.42 | A | C |
| ATOM | 291 | NE | ARG A | 143 | 49.470 | 18.634 | 122.168 | 1.00 | 12.17 | A | N |
| ATOM | 292 | CZ | ARG A | 143 | 50.164 | 19.671 | 122.631 | 1.00 | 14.73 A | C | |
| ATOM | 293 | NH1 | ARG A | 143 | 49.732 | 20.910 | 122.416 | 1.00 | 12.72 | A | N |
| ATOM | 294 | NH2 | ARG A | 143 | 51.305 | 19.471 | 123.287 | 1.00 | 14.62 | A | N |
| ATOM | 295 | C | ARG A | 143 | 50.139 | 20.214 | 116.952 | 1.00 | 16.25 | A | C |
| ATOM | 296 | O | ARG A | 143 | 49.478 | 21.162 | 116.535 | 1.00 | 18.41 | A | O |
| ATOM | 297 | N | ALA A | 144 | 51.453 | 20.140 | 116.787 | 1.00 | 14.76 | A | N |
| ATOM | 298 | CA | ALA A | 144 | 52.165 | 21.199 | 116.079 | 1.00 | 14.81 | A | C |
| ATOM | 299 | CB | ALA A | 144 | 53.687 | 21.021 | 116.247 | 1.00 | 9.68 | A | C |
| ATOM | 300 | C | ALA A | 144 | 51.763 | 21.169 | 114.592 | 1.00 | 15.68 | A | C |
| ATOM | 301 | O | ALA A | 144 | 51.588 | 22.214 | 113.970 | 1.00 | 14.69 | A | O |
| ATOM | 302 | N | PHE A | 145 | 51.605 | 19.968 | 114.033 | 1.00 | 16.80 | A | N |
| ATOM | 303 | CA | PHE A | 145 | 51.193 | 19.815 | 112.632 | 1.00 | 17.22 | A | C |
| ATOM | 304 | CB | PHE A | 145 | 51.386 | 18.367 | 112.154 | 1.00 | 14.07 | A | C |
| ATOM | 305 | CG | PHE A | 145 | 52.791 | 18.045 | 111.744 | 1.00 | 9.59 | A | C |
| ATOM | 306 | CD1 | PHE A | 145 | 53.433 | 18.797 | 110.767 | 1.00 | 10.98 | A | C |
| ATOM | 307 | CD2 | PHE A | 145 | 53.472 | 16.990 | 112.326 | 1.00 | 9.63 | A | C |
| ATOM | 308 | CE1 | PHE A | 145 | 54.739 | 18.503 | 110.373 | 1.00 | 10.38 | A | C |
| ATOM | 309 | CE2 | PHE A | 145 | 54.781 | 16.687 | 111.939 | 1.00 | 11.84 | A | C |
| ATOM | 310 | CZ | PHE A | 145 | 55.414 | 17.448 | 110.958 | 1.00 | 9.71 | A | C |
| ATOM | 311 | C | PHE A | 145 | 49.729 | 20.209 | 112.438 | 1.00 | 19.32 | A | C |
| ATOM | 312 | O | PHE A | 145 | 49.366 | 20.790 | 111.414 | 1.00 | 20.25 | A | O |
| ATOM | 313 | N | ALA A | 146 | 48.889 | 19.891 | 113.420 | 1.00 | 18.99 | A | N |
| ATOM | 314 | CA | ALA A | 146 | 47.471 | 20.224 | 113.330 | 1.00 | 20.15 | A | C |
| ATOM | 315 | CB | ALA A | 146 | 46.730 | 19.695 | 114.542 | 1.00 | 16.75 | A | C |
| ATOM | 316 | C | ALA A | 146 | 47.246 | 21.727 | 113.195 | 1.00 | 21.23 | A | C |
| ATOM | 317 | O | ALA A | 146 | 46.272 | 22.153 | 112.592 | 1.00 | 24.43 | A | O |
| ATOM | 318 | N | LEU A | 147 | 48.137 | 22.533 | 113.764 | 1.00 | 23.09 | A | N |
| ATOM | 319 | CA | LEU A | 147 | 47.996 | 23.983 | 113.668 | 1.00 | 21.92 | A | C |
| ATOM | 320 | CB | LEU A | 147 | 49.155 | 24.698 | 114.367 | 1.00 | 20.86 | A | C |
| ATOM | 321 | CG | LEU A | 147 | 49.260 | 24.748 | 115.894 | 1.00 | 24.92 | A | C |
| ATOM | 322 | CD1 | LEU A | 147 | 50.469 | 25.598 | 116.255 | 1.00 | 22.84 | A | C |
| ATOM | 323 | CD2 | LEU A | 147 | 48.001 | 25.342 | 116.513 | 1.00 | 22.59 | A | C |
| ATOM | 324 | C | LEU A | 147 | 47.984 | 24.422 | 112.206 | 1.00 | 22.21 | A | C |
| ATOM | 325 | O | LEU A | 147 | 47.188 | 25.265 | 111.797 | 1.00 | 22.01 | A | O |
| ATOM | 326 | N | TRP A | 148 | 48.884 | 23.842 | 111.425 | 1.00 | 21.86 | A | N |
| ATOM | 327 | CA | TRP A | 148 | 49.013 | 24.196 | 110.026 | 1.00 | 23.11 | A | C |
| ATOM | 328 | CB | TRP A | 148 | 50.413 | 23.831 | 109.534 | 1.00 | 19.89 | A | C |
| ATOM | 329 | CG | TRP A | 148 | 51.513 | 24.569 | 110.249 | 1.00 | 21.33 | A | C |
| ATOM | 330 | CD2 | TRP A | 148 | 51.827 | 25.964 | 110.138 | 1.00 | 21.93 | A | C |
| ATOM | 331 | CR2 | TRP A | 148 | 52.965 | 26.203 | 110.942 | 1.00 | 20.27 | A | C |
| ATOM | 332 | CR3 | TRP A | 148 | 51.259 | 27.036 | 109.435 | 1.00 | 18.40 | A | C |
| ATOM | 333 | CD1 | TRP A | 148 | 52.445 | 24.037 | 111.100 | 1.00 | 20.02 | A | C |
| ATOM | 334 | NE1 | TRP A | 148 | 53.320 | 25.012 | 111.517 | 1.00 | 17.63 | A | N |
| ATOM | 335 | CZ2 | TRP A | 148 | 53.548 | 27.472 | 111.059 | 1.00 | 19.95 | A | C |
| ATOM | 336 | CZ3 | TRP A | 148 | 51.838 | 28.297 | 109.553 | 1.00 | 19.74 | A | C |
| ATOM | 337 | CH2 | TRP A | 148 | 52.972 | 28.503 | 110.359 | 1.00 | 18.39 | A | C |
| ATOM | 338 | C | TRP A | 148 | 47.955 | 23.571 | 109.126 | 1.00 | 23.44 | A | C |
| ATOM | 339 | O | TRP A | 148 | 47.419 | 24.237 | 108.244 | 1.00 | 21.73 | A | O |
| ATOM | 340 | N | SER A | 149 | 47.645 | 22.300 | 109.351 | 1.00 | 25.55 | A | N |
| ATOM | 341 | CA | SER A | 149 | 46.648 | 21.630 | 108.531 | 1.00 | 27.95 | A | C |
| ATOM | 342 | CB | SER A | 149 | 46.618 | 20.127 | 108.823 | 1.00 | 28.12 | A | C |
| ATOM | 343 | OG | SER A | 149 | 46.132 | 19.877 | 110.124 | 1.00 | 27.95 | A | O |
| ATOM | 344 | C | SER A | 149 | 45.274 | 22.220 | 108.782 | 1.00 | 28.65 | A | C |
| ATOM | 345 | O | SER A | 149 | 44.340 | 21.959 | 108.031 | 1.00 | 30.51 | A | O |
| ATOM | 346 | N | ALA A | 150 | 45.148 | 23.022 | 109.833 | 1.00 | 27.46 | A | N |
| ATOM | 347 | CA | ALA A | 150 | 43.864 | 23.627 | 110.151 | 1.00 | 27.51 | A | C |
| ATOM | 348 | CB | ALA A | 150 | 43.817 | 24.013 | 111.620 | 1.00 | 24.47 | A | C |
| ATOM | 349 | C | ALA A | 150 | 43.552 | 24.843 | 109.280 | 1.00 | 28.60 | A | C |
| ATOM | 350 | O | ALA A | 150 | 42.393 | 25.187 | 109.091 | 1.00 | 28.82 | A | O |
| ATOM | 351 | N | VAL A | 151 | 44.579 | 25.492 | 108.745 | 1.00 | 30.95. | A | N |
| ATOM | 352 | CA | VAL A | 151 | 44.366 | 26.670 | 107.909 | 1.00 | 30.77 | A | C |
| ATOM | 353 | CB | VAL A | 151 | 45.144 | 27.891 | 108.456 | 1.00 | 30.72 | A | C |
| ATOM | 354 | OG1 | VAL A | 151 | 44.557 | 28.320 | 109.784 | 1.00 | 32.55 | A | C |
| ATOM | 355 | CG2 | VAL A | 151 | 46.617 | 27.546 | 108.623 | 1.00 | 27.92 | A | C |
| ATOM | 356 | C | VAL A | 151 | 44.765 | 26.451 | 106.455 | 1.00 | 30.85 | A | C |
| ATOM | 357 | O | VAL A | 151 | 44.898 | 27.405 | 105.691 | 1.00 | 31.86 | A | O |
| ATOM | 358 | N | THR A | 152 | 44.958 | 25.195 | 106.072 | 1.00 | 30.06 | A | N |
| ATOM | 359 | CA | THR A | 152 | 45.339 | 24.879 | 104.703 | 1.00 | 30.51 | A | C |
| ATOM | 360 | CB | THR A | 152 | 46.879 | 24.805 | 104.536 | 1.00 | 29.92 | A | C |
| ATOM | 361 | OG1 | THR A | 152 | 47.390 | 23.686 | 105.273 | 1.00 | 28.72 | A | O |
| ATOM | 362 | CG2 | THR A | 152 | 47.538 | 26.087 | 105.021 | 1.00 | 30.53 | A | C |
| ATOM | 363 | C | THR A | 152 | 44.760 | 23.530 | 104.301 | 1.00 | 30.44 | A | C |
| ATOM | 364 | O | THR A | 152 | 44.171 | 22.831 | 105.125 | 1.00 | 29.70 | A | O |
| ATOM | 365 | N | PRO A | 153 | 44.904 | 23.157 | 103.017 | 1.00 | 30.47 | A | N |
| ATOM | 366 | CD | PRO A | 153 | 45.298 | 24.023 | 101.887 | 1.00 | 30.45 | A | C |
| ATOM | 367 | CA | PRO A | 153 | 44.392 | 21.875 | 102.527 | 1.00 | 30.14 | A | C |
| ATOM | 368 | CB | PRO A | 153 | 44.174 | 22.145 | 101.042 | 1.00 | 30.77 | A | C |
| ATOM | 369 | CG | PRO A | 153 | 45.304 | 23.059 | 100.717 | 1.00 | 32.01 | A | C |
| ATOM | 370 | C | PRO A | 153 | 45.400 | 20.750 | 102.768 | 1.00 | 29.51. | A | C |
| ATOM | 371 | O | PRO A | 153 | 45.190 | 19.619 | 102.336 | 1.00 | 30.25 | A | O |
| ATOM | 372 | N | LEU A | 154 | 46.496 | 21.075 | 103.451 | 1.00 | 27.86 | A | N |
| ATOM | 373 | CA | LEU A | 154 | 47.539 | 20.100 | 103.753 | 1.00 | 26.19 | A | C |
| ATOM | 374 | CB | LEU A | 154 | 48.808 | 20.805 | 104.233 | 1.00 | 26.31 | A | C |
| ATOM | 375 | CG | LEU A | 154 | 49.491 | 21.847 | 103.342 | 1.00 | 25.08 | A | C |
| ATOM | 376 | CD1 | LEU A | 154 | 50.606 | 22.524 | 104.124 | 1.00 | 23.11 | A | C |
| ATOM | 377 | CD2 | LEU A | 154 | 50.043 | 21.196 | 102.097 | 1.00 | 26.17 | A | C |
| ATOM | 378 | C | LEU A | 154 | 47.104 | 19.116 | 104.832 | 1.00 | 25.56 | A | C |
| ATOM | 379 | O | LEU A | 154 | 46.290 | 19.440 | 105.693 | 1.00 | 26.20 | A | O |
| ATOM | 380 | N | THR A | 155 | 47.646 | 17.908 | 104.764 | 1.00 | 24.90 | A | N |
| ATOM | 381 | CA | THR A | 155 | 47.363 | 16.875 | 105.752 | 1.00 | 26.31 | A | C |
| ATOM | 382 | CB | THR A | 155 | 46.481 | 15.746 | 105.190 | 1.00 | 27.72 | A | C |
| ATOM | 383 | OD1 | THR A | 155 | 47.094 | 15.203 | 104.014 | 1.00 | 32.34 | A | O |
| ATOM | 384 | CG2 | THR A | 155 | 45.088 | 16.267 | 104.862 | 1.00 | 27.86 | A | C |
| ATOM | 385 | C | THR A | 155 | 48.711 | 16.294 | 106.134 | 1.00 | 25.34 | A | C |
| ATOM | 386 | O | THR A | 155 | 49.670 | 16.379 | 105.360 | 1.00 | 23.30 | A | O |
| ATOM | 387 | N | PHE A | 156 | 48.790 | 15.707 | 107.323 | 1.00 | 23.87 | A | N |
| ATOM | 388 | CA | PHE A | 156 | 50.044 | 15.139 | 107.787 | 1.00 | 22.82 | A | C |
| ATOM | 389 | CB | PHE A | 156 | 50.630 | 16.046 | 108.870 | 1.00 | 19.73 | A | C |
| ATOM | 390 | CG | PHE A | 156 | 50.935 | 17.434 | 108.375 | 1.00 | 15.72 | A | C |
| ATOM | 391 | CD1 | PHE A | 156 | 52.108 | 17.693 | 107.676 | 1.00 | 15.50 | A | C |
| ATOM | 392 | CD2 | PHE A | 156 | 50.011 | 18.461 | 108.536 | 1.00 | 16.19 | A | C |
| ATOM | 393 | CB1 | PHE A | 156 | 52.357 | 18.952 | 107.139 | 1.00 | 14.40 | A | C |
| ATOM | 394 | CE2 | PHE A | 156 | 50.249 | 19.725 | 108.004 | 1.00 | 15.79 | A | C |
| ATOM | 395 | CZ | PHE A | 156 | 51.423 | 19.972 | 107.304 | 1.00 | 15.01 | A | C |
| ATOM | 396 | C | PHE A | 156 | 49.864 | 13.718 | 108.286 | 1.00 | 24.14 | A | C |
| ATOM | 397 | O | PHE A | 156 | 49.026 | 13.441 | 109.145 | 1.00 | 26.34 | A | O |
| ATOM | 398 | N | THR A | 157 | 50.660 | 12.819 | 107.726 | 1.00 | 24.42 | A | N |
| ATOM | 399 | CA | THR A | 157 | 50.595 | 11.410 | 108.070 | 1.00 | 23.69 | A | C |
| ATOM | 400 | CB | THR A | 157 | 50.245 | 10.569 | 106.822 | 1.00 | 23.41 | A | C |
| ATOM | 401 | OG1 | THR A | 157 | 48.935 | 10.931 | 106.373 | 1.00 | 25.91 | A | O |
| ATOM | 402 | CG2 | THR A | 157 | 50.277 | 9.081 | 107.135 | 1.00 | 21.00 | A | C |
| ATOM | 403 | C | THR A | 157 | 51.915 | 10.939 | 108.638 | 1.00 | 22.11 | A | C |
| ATOM | 404 | O | THR A | 157 | 52.984 | 11.228 | 108.090 | 1.00 | 21.92 | A | O |
| ATOM | 405 | N | ARG A | 158 | 51.830 | 10.214 | 109.745 | 1.00 | 22.59 | A | N |
| ATOM | 406 | CA | ARG A | 158 | 53.015 | 9.679 | 110.394 | 1.00 | 22.35 | A | C |
| ATOM | 407 | CB | ARG A | 158 | 52.780 | 9.534 | 111.899 | 1.00 | 21.18 | A | C |
| ATOM | 408 | CG | ARG A | 158 | 53.998 | 9.010 | 112.630 | 1.00 | 21.22 | A | C |
| ATOM | 409 | CD | ARG A | 158 | 53.758 | 8.843 | 114.113 | 1.00 | 20.30 | A | C |
| ATOM | 410 | NE | ARG A | 158 | 54.959 | 8.340 | 114.776 | 1.00 | 20.99 | A | N |
| ATOM | 411 | CZ | ARG A | 158 | 55.013 | 7.974 | 116.051 | 1.00 | 20.64 | A | C |
| ATOM | 412 | NH1 | ARG A | 158 | 53.931 | 8.058 | 116.811 | 1.00 | 19.84 | A | N |
| ATOM | 413 | NH2 | ARG A | 158 | 56.147 | 7.506 | 116.560 | 1.00 | 20.74 | A | N |
| ATOM | 414 | C | ARG A | 158 | 53.362 | 8.320 | 109.802 | 1.00 | 21.92 | A | C |
| ATOM | 415 | O | ARG A | 158 | 52.508 | 7.442 | 109.710 | 1.00 | 21.61 | A | O |
| ATOM | 416 | N | VAL A | 159 | 54.618 | 8.156 | 109.399 | 1.00 | 24.22 | A | N |
| ATOM | 417 | CA | VAL A | 159 | 55.092 | 6.901 | 108.823 | 1.00 | 25.52 | A | C |
| ATOM | 418 | CB | VAL A | 159 | 55.403 | 7.059 | 107.311 | 1.00 | 23.23 | A | C |
| ATOM | 419 | CG1 | VAL A | 159 | 54.147 | 7.523 | 106.573 | 1.00 | 19.77 | A | C |
| ATOM | 420 | CG2 | VAL A | 159 | 56.539 | 8.045 | 107.109 | 1.00 | 21.91 | A | C |
| ATOM | 421 | C | VAL A | 159 | 56.344 | 6.469 | 109.579 | 1.00 | 28.57 | A | C |
| ATOM | 422 | O | VAL A | 159 | 56.842 | 7.212 | 110.424 | 1.00 | 29.72 | A | O |
| ATOM | 423 | N | TYR A | 160 | 56.859 | 5.278 | 109.282 | 1.00 | 31.79 | A | N |
| ATOM | 424 | CA | TYR A | 160 | 58.033 | 4.791 | 109.995 | 1.00 | 33.61 | A | C |
| ATOM | 425 | CB | TYR A | 160 | 57.634 | 3.596 | 110.863 | 1.00 | 29.27 | A | C |
| ATOM | 426 | CG | TYR A | 160 | 56.570 | 3.952 | 111.873 | 1.00 | 28.93 | A | C |
| ATOM | 427 | CD1 | TYR A | 160 | 55.230 | 4.056 | 111.498 | 1.00 | 28.23 | A | C |
| ATOM | 428 | CE1 | TYR A | 160 | 54.256 | 4.465 | 112.410 | 1.00 | 29.32 | A | C |
| ATOM | 429 | CD2 | TYR A | 160 | 56.912 | 4.261 | 113.191 | 1.00 | 28.64 | A | C |
| ATOM | 430 | CE2 | TYR A | 160 | 55.949 | 4.670 | 114.108 | 1.00 | 29.60 | A | C |
| ATOM | 431 | CZ | TYR A | 160 | 54.626 | 4.772 | 113.712 | 1.00 | 30.04 | A | C |
| ATOM | 432 | OH | TYR A | 160 | 53.680 | 5.193 | 114.616 | 1.00 | 32.56 | A | O |
| ATOM | 433 | C | TYR A | 160 | 59.270 | 4.449 | 109.170 | 1.00 | 36.31 | A | C |
| ATOM | 434 | O | TYR A | 160 | 60.097 | 3.640 | 109.591 | 1.00 | 39.18 | A | O |
| ATOM | 435 | N | SER A | 161 | 59.414 | 5.075 | 108.010 | 1.00 | 37.86 | A | N |
| ATOM | 436 | CA | SER A | 161 | 60.574 | 4.816 | 107.174 | 1.00 | 39.80 | A | C |
| ATOM | 437 | CB | SER A | 161 | 60.166 | 4.018 | 105.933 | 1.00 | 38.63 | A | C |
| ATOM | 438 | OG | SER A | 161 | 59.238 | 4.737 | 105.143 | 1.00 | 39.02 | A | O |
| ATOM | 439 | C | SER A | 161 | 61.231 | 6.128 | 106.766 | 1.00 | 41.58 | A | C |
| ATOM | 440 | O | SER A | 161 | 60.664 | 7.200 | 106.972 | 1.00 | 41.79 | A | O |
| ATOM | 441 | N | ARG A | 162 | 62.426 | 6.033 | 106.187 | 1.00 | 44.11 | A | N |
| ATOM | 442 | CA | ARG A | 162 | 63.188 | 7.198 | 105.739 | 1.00 | 46.57 | A | C |
| ATOM | 443 | CB | ARG A | 162 | 64.613 | 6.790 | 105.350 | 1.00 | 48.67 | A | C |
| ATOM | 444 | CG | ARG A | 162 | 65.408 | 6.045 | 106.418 | 1.00 | 53.36 | A | C |
| ATOM | 445 | CD | ARG A | 162 | 64.757 | 4.721 | 106.797 | 1.00 | 57.23 | A | C |
| ATOM | 446 | NE | ARG A | 162 | 65.661 | 3.839 | 107.528 | 1.00 | 60.31 | A | N |
| ATOM | 447 | CZ | ARG A | 162 | 65.274 | 2.728 | 108.145 | 1.00 | 62.63 | A | C |
| ATOM | 448 | NH1 | ARG A | 162 | 63.997 | 2.372 | 108.120 | 1.00 | 63.79 | A | N |
| ATOM | 449 | NH2 | ARG A | 162 | 66.161 | 1.965 | 108.777 | 1.00 | 62.58 | A | N |
| ATOM | 450 | C | ARG A | 162 | 62.537 | 7.862 | 104.529 | 1.00 | 47.42 | A | C |
| ATOM | 451 | O | ARG A | 162 | 63.027 | 8.879 | 104.040 | 1.00 | 48.56 | A | O |
| ATOM | 452 | N | ASP A | 163 | 61.441 | 7.282 | 104.048 | 1.00 | 48.01 | A | N. |
| ATOM | 453 | CA | ASP A | 163 | 60.739 | 7.804 | 102.876 | 1.00 | 46.94 | A | C |
| ATOM | 454 | CB | ASP A | 163 | 59.909 | 6.695 | 102.227 | 1.00 | 52.93 | A | C |
| ATOM | 455 | CG | ASP A | 163 | 60.766 | 5.584 | 101.665 | 1.00 | 56.71 | A | C |
| ATOM | 456 | OD1 | ASP A | 163 | 61.681 | 5.891 | 100.871 | 1.00 | 59.29 | A | O |
| ATOM | 457 | OD2 | ASP A | 163 | 60.525 | 4.409 | 102.013 | 1.00 | 61.03 | A | O |
| ATOM | 458 | C | ASP A | 163 | 59.838 | 9.008 | 103.127 | 1.00 | 42.48 | A | C |
| ATOM | 459 | O | ASP A | 163 | 59.350 | 9.627 | 102.181 | 1.00 | 43.42 | A | O |
| ATOM | 460 | N | ALA A | 164 | 59.612 | 9.337 | 104.392 | 1.00 | 35.33 | A | N |
| ATOM | 461 | CA | ALA A | 164 | 58.768 | 10.476 | 104.738 | 1.00 | 28.19 | A | C |
| ATOM | 462 | CB | ALA A | 164 | 58.656 | 10.591 | 106.254 | 1.00 | 28.52 | A | C |
| ATOM | 463 | C | ALA A | 164 | 59.332 | 11.776 | 104.159 | 1.00 | 24.32 | A | C |
| ATOM | 464 | O | ALA A | 164 | 60.528 | 11.868 | 103.859 | 1.00 | 18.94 | A | O |
| ATOM | 465 | N | ASP A | 165 | 58.468 | 12.776 | 103.996 | 1.00 | 20.55 | A | N |
| ATOM | 466 | CA | ASP A | 165 | 58.910 | 14.070 | 103.491 | 1.00 | 19.96 | A | C |
| ATOM | 467 | CB | ASP A | 165 | 57.732 | 14.931 | 103.033 | 1.00 | 20.58 | A | C |
| ATOM | 468 | CG | ASP A | 165 | 56.953 | 14.302 | 101.918 | 1.00 | 19.34 | A | C |
| ATOM | 469 | OD1 | ASP A | 165 | 57.577 | 13.931 | 100.907 | 1.00 | 22.92 | A | O |
| ATOM | 470 | OD2 | ASP A | 165 | 55.720 | 14.185 | 102.053 | 1.00 | 20.26 | A | O |
| ATOM | 471 | C | ASP A | 165 | 59.619 | 14.808 | 104.609 | 1.00 | 19.50 | A | C |
| ATOM | 472 | O | ASP A | 165 | 60.669 | 15.387 | 104.403 | 1.00 | 20.89 | A | O |
| ATOM | 473 | N | ILE A | 166 | 59.031 | 14.798 | 105.798 | 1.00 | 18.88 | A | N |
| ATOM | 474 | CA | ILE A | 166 | 59.638 | 15.496 | 106.923 | 1.00 | 18.31 | A | C |
| ATOM | 475 | CB | ILE A | 166 | 58.612 | 16.435 | 107.598 | 1.00 | 16.76 | A | C |
| ATOM | 476 | CG2 | ILE A | 166 | 59.241 | 17.114 | 108.817 | 1.00 | 15.02 | A | C |
| ATOM | 477 | CG1 | ILE A | 166 | 58.129 | 17.477 | 106.581 | 1.00 | 16.47 | A | C |
| ATOM | 478 | CD1 | ILE A | 166 | 57.004 | 18.367 | 107.073 | 1.00 | 12.28 | A | C |
| ATOM | 479 | C | ILE A | 166 | 60.198 | 14.523 | 107.950 | 1.00 | 18.31 | A | C |
| ATOM | 480 | O | ILE A | 166 | 59.448 | 13.831 | 108.635 | 1.00 | 19.42 | A | O |
| ATOM | 481 | N | VAL A | 167 | 61.522 | 14.453 | 108.032 | 1.00 | 16.42 | A | N |
| ATOM | 482 | CA | VAL A | 167 | 62.172 | 13.577 | 108.992 | 1.00 | 14.89 | A | C |
| ATOM | 483 | CB | VAL A | 167 | 63.410 | 12.891 | 108.391 | 1.00 | 16.42 | A | C |
| ATOM | 484 | CG1 | VAL A | 167 | 64.133 | 12.076 | 109.464 | 1.00 | 14.30 | A | C |
| ATOM | 485 | CG2 | VAL A | 167 | 62.984 | 11.982 | 107.243 | 1.00 | 18.08 | A | C |
| ATOM | 486 | C | VAL A | 167 | 62.581 | 14.415 | 110.200 | 1.00 | 17.08 | A | C |
| ATOM | 487 | O | VAL A | 167 | 63.232 | 15.456 | 110.064 | 1.00 | 13.46 | A | O |
| ATOM | 488 | N | ILE A | 168 | 62.183 | 13.943 | 111.378 | 1.00 | 15.49 | A | N |
| ATOM | 489 | CA | ILE A | 168 | 62.445 | 14.624 | 112.633 | 1.00 | 16.01 | A | C |
| ATOM | 490 | CB | ILE A | 168 | 61.145 | 14.735 | 113.448 | 1.00 | 13.64 | A | C |
| ATOM | 491 | CG2 | ILE A | 168 | 61.406 | 15.473 | 114.755 | 1.00 | 14.98 | A | C |
| ATOM | 492 | CG1 | ILE A | 168 | 60.077 | 15.427 | 112.603 | 1.00 | 12.69 | A | C |
| ATOM | 493 | CD1 | ILE A | 168 | 58.685 | 15.344 | 113.170 | 1.00 | 10.54 | A | C |
| ATOM | 494 | C | ILE A | 168 | 63.482 | 13.906 | 113.486 | 1.00 | 16.29 | A | C |
| ATOM | 495 | O | ILE A | 168 | 63.442 | 12.693 | 113.648 | 1.00 | 18.46 | A | O |
| ATOM | 496 | N | GLN A | 169 | 64.414 | 14.656 | 114.045 | 1.00 | 19.10 | A | N |
| ATOM | 497 | CA | GLN A | 169 | 65.407 | 14.028 | 114.890 | 1.00 | 20.39 | A | C |
| ATOM | 498 | CB | GLN A | 169 | 66.536 | 13.443 | 114.043 | 1.00 | 23.58 | A | C |
| ATOM | 499 | CG | GLN A | 169 | 67.428 | 14.481 | 113.409 | 1.00 | 28.43 | A | C |
| ATOM | 500 | CD | GLN A | 169 | 68.723 | 13.890 | 112.901 | 1.00 | 32.40 | A | C |
| ATOM | 501 | OE1 | GLN A | 169 | 69.551 | 13.411 | 113.678 | 1.00 | 36.26 | A | O |
| ATOM | 502 | NE2 | GLN A | 169 | 68.908 | 13.917 | 111.591 | 1.00 | 33.78 | A | N |
| ATOM | 503 | C | GLN A | 169 | 65.998 | 14.991 | 115.905 | 1.00 | 19.32 | A | C |
| ATOM | 504 | O | GLN A | 169 | 66.009 | 16.206 | 115.694 | 1.00 | 17.87 | A | O |
| ATOM | 505 | N | PHE A | 170 | 66.482 | 14.423 | 117.005 | 1.00 | 15.27 | A | N |
| ATOM | 506 | CA | PHE A | 170 | 67.124 | 15.180 | 118.062 | 1.00 | 16.05 | A | C |
| ATOM | 507 | CB | PHE A | 170 | 66.613 | 14.740 | 119.444 | 1.00 | 13.11 | A | C |
| ATOM | 508 | CG | PHE A | 170 | 65.164 | 15.056 | 119.690 | 1.00 | 11.58 | A | C |
| ATOM | 509 | CD1 | PHE A | 170 | 64.157 | 14.272 | 119.131 | 1.00 | 9.60 | A | C |
| ATOM | 510 | CD2 | PHE A | 170 | 64.807 | 16.148 | 120.475 | 1.00 | 11.94 | A | C |
| ATOM | 511 | CE1 | PHE A | 170 | 62.817 | 14.572 | 119.352 | 1.00 | 10.10 | A | C |
| ATOM | 512 | CE2 | PHE A | 170 | 63.466 | 16.458 | 120.701 | 1.00 | 11.51 | A | C |
| ATOM | 513 | CZ | PHE A | 170 | 62.471 | 15.671 | 120.141 | 1.00 | 11.15 | A | C |
| ATOM | 514 | C | PHE A | 170 | 68.611 | 14.873 | 117.953 | 1.00 | 17.13 | A | C |
| ATOM | 515 | O | PHE A | 170 | 68.998 | 13.728 | 117.747 | 1.00 | 18.15 | A | O |
| ATOM | 516 | N | GLY A | 171 | 69.450 | 15.889 | 118.082 | 1.00 | 17.88 | A | N |
| ATOM | 517 | CA | GLY A | 171 | 70.879 | 15.655 | 118.006 | 1.00 | 19.53 | A | C |
| ATOM | 518 | C | GLY A | 171 | 71.642 | 16.630 | 118.878 | 1.00 | 21.64 | A | C |
| ATOM | 519 | O | GLY A | 171 | 71.083 | 17.631 | 119.324 | 1.00 | 22.76 | A | O |
| ATOM | 520 | N | VAL A | 172 | 72.912 | 16.332 | 119.138 | 1.00 | 23.09 | A | N |
| ATOM | 521 | CA | VAL A | 172 | 73.756 | 17.205 | 119.950 | 1.00 | 25.50 | A | C |
| ATOM | 522 | CB | VAL A | 172 | 74.109 | 16.560 | 121.326 | 1.00 | 26.03 | A | C |
| ATOM | 523 | CG1 | VAL A | 172 | 72.844 | 16.252 | 122.083 | 1.00 | 26.98 | A | C |
| ATOM | 524 | CG2 | VAL A | 172 | 74.918 | 15.292 | 121.134 | 1.00 | 25.99 | A | C |
| ATOM | 525 | C | VAL A | 172 | 75.038 | 17.489 | 119.174 | 1.00 | 26.07 | A | C |
| ATOM | 526 | O | VAL A | 172 | 75.653 | 16.574 | 118.615 | 1.00 | 24.44 | A | O |
| ATOM | 527 | N | ALA A | 173 | 75.427 | 18.760 | 119.139 | 1.00 | 27.68 | A | N |
| ATOM | 528 | CA | ALA A | 173 | 76.618 | 19.184 | 118.411 | 1.00 | 28.80 | A | C |
| ATOM | 529 | CB | ALA A | 173 | 77.868 | 18.664 | 119.111 | 1.00 | 29.58 | A | C |
| ATOM | 530 | C | ALA A | 173 | 76.548 | 18.659 | 116.974 | 1.00 | 29.57 | A | C |
| ATOM | 531 | O | ALA A | 173 | 75.538 | 18.843 | 116.289 | 1.00 | 27.64 | A | O |
| ATOM | 532 | N | GLU A | 174 | 77.619 | 18.006 | 116.530 | 1.00 | 29.27 | A | N |
| ATOM | 533 | CA | GLU A | 174 | 77.699 | 17.437 | 115.189 | 1.00 | 32.29 | A | C |
| ATOM | 534 | CB | GLU A | 174 | 79.078 | 16.815 | 114.972 | 1.00 | 41.88 | A | C |
| ATOM | 535 | CG | GLU A | 174 | 80.209 | 17.511 | 115.722 | 1.00 | 51.63 | A | C |
| ATOM | 536 | CD | GLU A | 174 | 80.512 | 18.892 | 115.178 | 1.00 | 57.77 | A | C |
| ATOM | 537 | OE1 | GLU A | 174 | 81.379 | 19.584 | 115.757 | 1.00 | 58.88 | A | O |
| ATOM | 538 | OE2 | GLU A | 174 | 79.888 | 19.284 | 114.167 | 1.00 | 62.24 | A | O |
| ATOM | 539 | C | GLU A | 174 | 76.657 | 16.340 | 115.076 | 1.00 | 30.20 | A | C |
| ATOM | 540 | O | GLU A | 174 | 76.668 | 15.409 | 115.874 | 1.00 | 31.68 | A | O |
| ATOM | 541 | N | HIS A | 175 | 75.768 | 16.418 | 114.092 | 1.00 | 27.49 | A | N |
| ATOM | 542 | CA | HIS A | 175 | 74.748 | 15.385 | 113.979 | 1.00 | 27.72 | A | C |
| ATOM | 543 | CB | HIS A | 175 | 73.473 | 15.827 | 114.688 | 1.00 | 24.97 | A | C |
| ATOM | 544 | CG | HIS A | 175 | 72.928 | 17.118 | 114.175 | 1.00 | 20.26 | A | C |
| ATOM | 545 | CD2 | HIS A | 175 | 72.023 | 17.379 | 113.203 | 1.00 | 17.16 | A | C |
| ATOM | 546 | ND1 | HIS A | 175 | 73.354 | 18.341 | 114.644 | 1.00 | 20.15 | A | N |
| ATOM | 547 | CE1 | HIS A | 175 | 72.734 | 19.302 | 113.982 | 1.00 | 18.26 | A | C |
| ATOM | 548 | NE2 | HIS A | 175 | 71.922 | 18.744 | 113.102 | 1.00 | 15.77 | A | N |
| ATOM | 549 | C | HIS A | 175 | 74.392 | 14.949 | 112.571 | 1.00 | 30.79 | A | C |
| ATOM | 550 | O | HIS A | 175 | 73.257 | 14.536 | 112.317 | 1.00 | 32.24 | A | O |
| ATOM | 551 | N | GLY A | 176 | 75.341 | 15.042 | 111.647 | 1.00 | 32.67 | A | N |
| ATOM | 552 | CA | GLY A | 176 | 75.048 | 14.601 | 110.297 | 1.00 | 34.93 | A | C |
| ATOM | 553 | C | GLY A | 176 | 75.221 | 15.634 | 109.213 | 1.00 | 36.14 | A | C |
| ATOM | 554 | O | GLY A | 176 | 76.066 | 15.481 | 108.340 | 1.00 | 37.19 | A | O |
| ATOM | 555 | N | ASP A | 177 | 74.399 | 16.674 | 109.240 | 1.00 | 37.27 | A | N |
| ATOM | 556 | CA | ASP A | 177 | 74.512 | 17.721 | 108.242 | 1.00 | 35.71 | A | C |
| ATOM | 557 | CB | ASP A | 177 | 73.267 | 18.600 | 108.231 | 1.00 | 34.06 | A | C |
| ATOM | 558 | CG | ASP A | 177 | 72.828 | 19.006 | 109.617 | 1.00 | 32.01 | A | C |
| ATOM | 559 | OD1 | ASP A | 177 | 73.684 | 19.394 | 110.439 | 1.00 | 28.22 | A | O |
| ATOM | 560 | OD2 | ASP A | 177 | 71.613 | 18.942 | 109.872 | 1.00 | 33.33 | A | O |
| ATOM | 561 | C | ASP A | 177 | 75.734 | 18.551 | 108.587 | 1.00 | 36.61 | A | C |
| ATOM | 562 | O | ASP A | 177 | 76.570 | 18.132 | 109.389 | 1.00 | 40.71 | A | O |
| ATOM | 563 | N | GLY A | 178 | 75.845 | 19.730 | 107.996 | 1.00 | 34.57 | A | N |
| ATOM | 564 | CA | GLY A | 178 | 77.008 | 20.546 | 108.278 | 1.00 | 36.70 | A | C |
| ATOM | 565 | C | GLY A | 178 | 76.753 | 21.721 | 109.194 | 1.00 | 36.80 | A | C |
| ATOM | 566 | O | GLY A | 178 | 77.520 | 22.684 | 109.184 | 1.00 | 37.18 | A | O |
| ATOM | 567 | N | TYR A | 179 | 75.688 | 21.649 | 109.991 | 1.00 | 37.02 | A | N |
| ATOM | 568 | CA | TYR A | 179 | 75.344 | 22.731 | 110.908 | 1.00 | 34.28 | A | C |
| ATOM | 569 | CB | TYR A | 179 | 74.078 | 23.436 | 110.425 | 1.00 | 36.20 | A | C |
| ATOM | 570 | CG | TYR A | 179 | 74.181 | 23.932 | 109.003 | 1.00 | 42.47 | A | C |
| ATOM | 571 | CD1 | TYR A | 179 | 73.949 | 23.076 | 107.927 | 1.00 | 45.68 | A | C |
| ATOM | 572 | CE1 | TYR A | 179 | 74.065 | 23.524 | 106.609 | 1.00 | 46.33 | A | C |
| ATOM | 573 | CD2 | TYR A | 179 | 74.536 | 25.253 | 108.728 | 1.00 | 44.79 | A | C |
| ATOM | 574 | CE2 | TYR A | 179 | 74.658 | 25.712 | 107.415 | 1.00 | 45.00 | A | C |
| ATOM | 575 | CZ | TYR A | 179 | 74.420 | 24.843 | 106.362 | 1.00 | 47.34 | A | C |
| ATOM | 576 | OH | TYR A | 179 | 74.528 | 25.293 | 105.062 | 1.00 | 48.94 | A | O |
| ATOM | 577 | C | TYR A | 179 | 75.153 | 22.235 | 112.335 | 1.00 | 31.65 | A | C |
| ATOM | 578 | O | TYR A | 179 | 74.033 | 22.165 | 112.838 | 1.00 | 29.30 | A | O |
| ATOM | 579 | N | PRO A | 180 | 76.260 | 21.895 | 113.014 | 1.00 | 31.79 | A | N |
| ATOM | 580 | CD | PRO A | 180 | 77.653 | 22.113 | 112.589 | 1.00 | 30.47 | A | C |
| ATOM | 581 | CA | PRO A | 180 | 76.212 | 21.401 | 114.394 | 1.00 | 30.83 | A | C |
| ATOM | 582 | CB | PRO A | 180 | 77.685 | 21.194 | 114.739 | 1.00 | 31.29 | A | C |
| ATOM | 583 | CG | PRO A | 180 | 78.370 | 22.238 | 113.916 | 1.00 | 31.47 | A | C |
| ATOM | 584 | C | PRO A | 180 | 75.532 | 22.381 | 115.335 | 1.00 | 28.21 | A | C |
| ATOM | 585 | O | PRO A | 180 | 75.505 | 23.579 | 115.073 | 1.00 | 28.60 | A | O |
| ATOM | 586 | N | PHE A | 181 | 74.971 | 21.866 | 116.422 | 1.00 | 25.93 | A | N |
| ATOM | 587 | CA | PHE A | 181 | 74.306 | 22.721 | 117.388 | 1.00 | 26.27 | A | C |
| ATOM | 588 | CB | PHE A | 181 | 73.238 | 21.937 | 118.158 | 1.00 | 23.67 | A | C |
| ATOM | 589 | CG | PHE A | 181 | 72.012 | 21.625 | 117.346 | 1.00 | 21.97 | A | C |
| ATOM | 590 | CD1 | PHE A | 181 | 71.262 | 22.649 | 116.778 | 1.00 | 22.39 | A | C. |
| ATOM | 591 | CD2 | PHE A | 181 | 71.590 | 20.309 | 117.165 | 1.00 | 21.41 | A | C |
| ATOM | 592 | CE1 | PHE A | 181 | 70.102 | 22.369 | 116.040 | 1.00 | 22.23 | A | C |
| ATOM | 593 | CE2 | PHE A | 181 | 70.432 | 20.019 | 116.430 | 1.00 | 20.84 | A | C |
| ATOM | 594 | CZ | PHE A | 181 | 69.688 | 21.052 | 115.868 | 1.00 | 19.15 | A | C |
| ATOM | 595 | C | PHE A | 181 | 75.351 | 23.306 | 118.335 | 1.00 | 28.41 | A | C |
| ATOM | 596 | O | PHE A | 181 | 76.498 | 22.863 | 118.354 | 1.00 | 27.60 | A | O |
| ATOM | 597 | N | ASP A | 182 | 74.943 | 24.290 | 119.127 | 1.00 | 28.53 | A | N |
| ATOM | 598 | CA | ASP A | 182 | 75.848 | 24.979 | 120.031 | 1.00 | 29.79 | A | C |
| ATOM | 599 | CB | ASP A | 182 | 75.723 | 26.478 | 119.769 | 1.00 | 28.81 | A | C |
| ATOM | 600 | CG | ASP A | 182 | 74.284 | 26.937 | 119.799 | 1.00 | 29.97 | A | C |
| ATOM | 601 | OD1 | ASP A | 182 | 73.464 | 26.193 | 120.358 | 1.00 | 30.97 | A | O |
| ATOM | 602 | OD2 | ASP A | 182 | 73.962 | 28.025 | 119.284 | 1.00 | 31.92 | A | O |
| ATOM | 603 | C | ASP A | 182 | 75.677 | 24.718 | 121.535 | 1.00 | 31.81 | A | C |
| ATOM | 604 | O | ASP A | 182 | 75.941 | 25.608 | 122.346 | 1.00 | 34.01 | A | O |
| ATOM | 605 | N | GLY A | 183 | 75.249 | 23.520 | 121.921 | 1.00 | 31.93 | A | N |
| ATOM | 606 | CA | GLY A | 183 | 75.085 | 23.236 | 123.341 | 1.00 | 31.05 | A | C |
| ATOM | 607 | C | GLY A | 183 | 73.943 | 24.014 | 123.976 | 1.00 | 32.23 | A | C |
| ATOM | 608 | O | GLY A | 183 | 73.094 | 24.524 | 123.276 | 1.00 | 32.15 | A | O |
| ATOM | 609 | N | LYS A | 184 | 73.923 | 24.122 | 125.299 | 1.00 | 32.11 | A | N |
| ATOM | 610 | CA | LYS A | 184 | 72.851 | 24.832 | 126.002 | 1.00 | 33.02 | A | C |
| ATOM | 611 | CB | LYS A | 184 | 73.209 | 24.984 | 127.484 | 1.00 | 37.17 | A | C |
| ATOM | 612 | CG | LYS A | 184 | 72.020 | 25.232 | 128.396 | 1.00 | 41.04 | A | C |
| ATOM | 613 | CD | LYS A | 184 | 71.101 | 24.017 | 128.419 | 1.00 | 44.80 | A | C |
| ATOM | 614 | CE | LYS A | 184 | 69.925 | 24.219 | 129.353 | 1.00 | 45.32 | A | C |
| ATOM | 615 | NZ | LYS A | 184 | 70.371 | 24.435 | 130.752 | 1.00 | 48.28 | A | N |
| ATOM | 616 | C | LYS A | 184 | 72.539 | 26.207 | 125.424 | 1.00 | 31.99 | A | C |
| ATOM | 617 | O | LYS A | 184 | 73.445 | 26.975 | 125.118 | 1.00 | 34.08 | A | O |
| ATOM | 618 | N | ASP A | 185 | 71.250 | 26.504 | 125.278 | 1.00 | 30.60 | A | N |
| ATOM | 619 | CA | ASP A | 185 | 70.777 | 27.787 | 124.756 | 1.00 | 29.01 | A | C |
| ATOM | 620 | CB | ASP A | 185 | 71.068 | 28.895 | 125.763 | 1.00 | 33.03 | A | C |
| ATOM | 621 | CG | ASP A | 185 | 70.304 | 28.712 | 127.046 | 1.00 | 36.57 | A | C |
| ATOM | 622 | OD1 | ASP A | 185 | 69.058 | 28.796 | 127.011 | 1.00 | 39.21 | A | O |
| ATOM | 623 | OD2 | ASP A | 185 | 70.947 | 28.470 | 128.090 | 1.00 | 40.55 | A | O |
| ATOM | 624 | C | ASP A | 185 | 71.311 | 28.198 | 123.394 | 1.00 | 26.92 | A | C |
| ATOM | 625 | O | ASP A | 185 | 71.999 | 27.438 | 122.727 | 1.00 | 25.74 | A | O |
| ATOM | 626 | N | GLY A | 186 | 70.992 | 29.422 | 122.990 | 1.00 | 24.58 | A | N |
| ATOM | 627 | CA | GLY A | 186 | 71.431 | 29.904 | 121.695 | 1.00 | 26.41 | A | C |
| ATOM | 628 | C | GLY A | 186 | 70.472 | 29.395 | 120.637 | 1.00 | 26.21 | A | C |
| ATOM | 629 | O | GLY A | 186 | 69.280 | 29.693 | 120.691 | 1.00 | 27.15 | A | O |
| ATOM | 630 | N | LEU A | 187 | 70.987 | 28.625 | 119.684 | 1.00 | 26.00 | A | N |
| ATOM | 631 | CA | LEU A | 187 | 70.166 | 28.058 | 118.622 | 1.00 | 25.00 | A | C |
| ATOM | 632 | CB | LEU A | 187 | 71.057 | 27.560 | 117.483 | 1.00 | 25.51 | A | C |
| ATOM | 633 | CG | LEU A | 187 | 70.323 | 27.068 | 116.233 | 1.00 | 31.44 | A | C |
| ATOM | 634 | CD1 | LEU A | 187 | 69.618 | 28.248 | 115.564 | 1.00 | 27.44 | A | C |
| ATOM | 635 | CD2 | LEU A | 187 | 71.313 | 26.409 | 115.271 | 1.00 | 32.87 | A | C |
| ATOM | 636 | C | LEU A | 187 | 69.394 | 26.885 | 119.220 | 1.00 | 24.47 | A | C |
| ATOM | 637 | O | LEU A | 187 | 69.996 | 25.979 | 119.775 | 1.00 | 23.75 | A | O |
| ATOM | 638 | N | LEU A | 188 | 68.071 | 26.881 | 119.097 | 1.00 | 22.38 | A | N |
| ATOM | 639 | CA | LEU A | 188 | 67.272 | 25.800 | 119.673 | 1.00 | 20.10 | A | C |
| ATOM | 640 | CB | LEU A | 188 | 65.930 | 26.349 | 120.158 | 1.00 | 17.21 | A | C |
| ATOM | 641 | CG | LEU A | 188 | 65.981 | 27.580 | 121.077 | 1.00 | 19.72 | A | C |
| ATOM | 642 | CD1 | LEU A | 188 | 64.565 | 27.997 | 121.447 | 1.00 | 14.44 | A | C |
| ATOM | 643 | CD2 | LEU A | 188 | 66.791 | 27.275 | 122.329 | 1.00 | 16.62 | A | C |
| ATOM | 644 | C | LEU A | 188 | 67.026 | 24.638 | 118.712 | 1.00 | 21.08 | A | C |
| ATOM | 645 | O | LEU A | 188 | 66.907 | 23.477 | 119.129 | 1.00 | 18.95 | A | O |
| ATOM | 646 | N | ALA A | 189 | 66.956 | 24.958 | 117.426 | 1.00 | 19.08 | A | N |
| ATOM | 647 | CA | ALA A | 189 | 66.703 | 23.963 | 116.400 | 1.00 | 16.54 | A | C |
| ATOM | 648 | CB | ALA A | 189 | 65.297 | 23.398 | 116.575 | 1.00 | 16.25 | A | C |
| ATOM | 649 | C | ALA A | 189 | 66.824 | 24.626 | 115.039 | 1.00 | 17.08 | A | C |
| ATOM | 650 | O | ALA A | 189 | 67.110 | 25.815 | 114.943 | 1.00 | 16.33 | A | O |
| ATOM | 651 | N | HIS A | 190 | 66.627 | 23.842 | 113.988 | 1.00 | 16.66 | A | N |
| ATOM | 652 | CA | HIS A | 190 | 66.656 | 24.361 | 112.636 | 1.00 | 18.31 | A | C |
| ATOM | 653 | CB | HIS A | 190 | 68.099 | 24.705 | 112.191 | 1.00 | 18.50 | A | C |
| ATOM | 654 | CG | HIS A | 190 | 69.055 | 23.550 | 112.164 | 1.00 | 17.26 | A | C |
| ATOM | 655 | CD2 | HIS A | 190 | 68.920 | 22.281 | 111.712 | 1.00 | 20.42 | A | C |
| ATOM | 656 | ND1 | HIS A | 190 | 70.369 | 23.671 | 112.567 | 1.00 | 18.18 | A | N |
| ATOM | 657 | CE1 | HIS A | 190 | 71.002 | 22.531 | 112.365 | 1.00 | 17.27 | A | C |
| ATOM | 658 | NB2 | HIS A | 190 | 70.145 | 21.671 | 111.846 | 1.00 | 18.89 | A | N |
| ATOM | 659 | C | HIS A | 190 | 65.982 | 23.372 | 111.699 | 1.00 | 18.42 | A | C |
| ATOM | 660 | O | HIS A | 190 | 65.985 | 22.168 | 111.956 | 1.00 | 18.06 | A | O |
| ATOM | 661 | N | ALA A | 191 | 65.367 | 23.887 | 110.637 | 1.00 | 19.52 | A | N |
| ATOM | 662 | CA | ALA A | 191 | 64.672 | 23.049 | 109.659 | 1.00 | 20.18 | A | C |
| ATOM | 663 | CB | ALA A | 191 | 63.163 | 23.158 | 109.853 | 1.00 | 17.02 | A | C |
| ATOM | 664 | C | ALA A | 191 | 65.052 | 23.460 | 108.242 | 1.00 | 22.20 | A | C |
| ATOM | 665 | O | ALA A | 191 | 65.547 | 24.563 | 108.026 | 1.00 | 22.82 | A | O |
| ATOM | 666 | N | PHE A | 192 | 64.822 | 22.569 | 107.279 | 1.00 | 24.96 | A | N |
| ATOM | 667 | CA | PHE A | 192 | 65.156 | 22.847 | 105.884 | 1.00 | 24.10 | A | C |
| ATOM | 668 | CB | PHE A | 192 | 66.013 | 21.724 | 105.303 | 1.00 | 24.41 | A | C |
| ATOM | 669 | CG | PHE A | 192 | 67.299 | 21.491 | 106.042 | 1.00 | 25.34 | A | C |
| ATOM | 670 | CD1 | PHE A | 192 | 67.328 | 20.696 | 107.184 | 1.00 | 27.98 | A | C |
| ATOM | 671 | CD2 | PHE A | 192 | 68.483 | 22.059 | 105.594 | 1.00 | 26.82 | A | C |
| ATOM | 672 | CE1 | PHE A | 192 | 68.524 | 20.466 | 107.870 | 1.00 | 27.32 | A | C |
| ATOM | 673 | CE2 | PHE A | 192 | 69.682 | 21.839 | 106.267 | 1.00 | 27.38 | A | C |
| ATOM | 674 | CZ | PHE A | 192 | 69.703 | 21.039 | 107.410 | 1.00 | 28.50 | A | C |
| ATOM | 675 | C | PHE A | 192 | 63.906 | 23.012 | 105.033 | 1.00 | 25.05 | A | C |
| ATOM | 676 | O | PHE A | 192 | 62.891 | 22.346 | 105.270 | 1.00 | 24.67 | A | O |
| ATOM | 677 | N | PRO A | 193 | 63.967 | 23.908 | 104.027 | 1.00 | 25.22 | A | N |
| ATOM | 678 | CD | PRO A | 193 | 65.156 | 24.708 | 103.684 | 1.00 | 25.93 | A | C |
| ATOM | 679 | CA | PRO A | 193 | 62.861 | 24.203 | 103.103 | 1.00 | 24.58 | A | C |
| ATOM | 680 | CB | PRO A | 193 | 63.434 | 25.292 | 102.195 | 1.00 | 22.65 | A | C |
| ATOM | 681 | CG | PRO A | 193 | 64.542 | 25.894 | 102.997 | 1.00 | 26.18 | A | C |
| ATOM | 682 | C | PRO A | 193 | 62.454 | 22.970 | 102.299 | 1.00 | 24.59 | A | C |
| ATOM | 683 | O | PRO A | 193 | 63.231 | 22.018 | 102.164 | 1.00 | 23.68 | A | O |
| ATOM | 684 | N | PRO A | 194 | 61.233 | 22.981 | 101.745 | 1.00 | 23.30 | A | N |
| ATOM | 685 | CD | PRO A | 194 | 60.243 | 24.072 | 101.820 | 1.00 | 20.97 | A | C |
| ATOM | 686 | CA | PRO A | 194 | 60.718 | 21.861 | 100.949 | 1.00 | 24.06 | A | C |
| ATOM | 687 | CB | PRO A | 194 | 59.445 | 22.441 | 100.334 | 1.00 | 22.11 | A | C |
| ATOM | 688 | CG | PRO A | 194 | 58.968 | 23.371 | 101.408 | 1.00 | 21.80 | A | C |
| ATOM | 689 | C | PRO A | 194 | 61.710 | 21.371 | 99.891 | 1.00 | 23.68 | A | C |
| ATOM | 690 | O | PRO A | 194 | 62.473 | 22.147 | 99.325 | 1.00 | 21.32 | A | O |
| ATOM | 691 | N | GLY A | 195 | 61.688 | 20.071 | 99.631 | 1.00 | 26.27 | A | N |
| ATOM | 692 | CA | GLY A | 195 | 62.584 | 19.500 | 98.645 | 1.00 | 26.15 | A | C |
| ATOM | 693 | C | GLY A | 195 | 62.908 | 18.059 | 98.978 | 1.00 | 28.20 | A | C |
| ATOM | 694 | O | GLY A | 195 | 62.355 | 17.505 | 99.926 | 1.00 | 29.23 | A | O |
| ATOM | 695 | N | PRO A | 196 | 63.801 | 17.420 | 98.211 | 1.00 | 28.08 | A | N |
| ATOM | 696 | CD | PRO A | 196 | 64.340 | 17.902 | 96.926 | 1.00 | 26.14 | A | C |
| ATOM | 697 | CA | PRO A | 196 | 64.188 | 16.025 | 98.445 | 1.00 | 28.94 | A | C |
| ATOM | 698 | CB | PRO A | 196 | 64.613 | 15.563 | 97.057 | 1.00 | 27.73 | A | C |
| ATOM | 699 | CG | PRO A | 196 | 65.288 | 16.788 | 96.526 | 1.00 | 26.49 | A | C |
| ATOM | 700 | C | PRO A | 196 | 65.320 | 15.876 | 99.472 | 1.00 | 29.39 | A | C |
| ATOM | 701 | O | PRO A | 196 | 65.972 | 16.855 | 99.837 | 1.00 | 31.26 | A | O |
| ATOM | 702 | N | GLY A | 197 | 65.542 | 14.646 | 99.930 | 1.00 | 28.16 | A | N |
| ATOM | 703 | CA | GLY A | 197 | 66.605 | 14.374 | 100.884 | 1.00 | 26.43 | A | C |
| ATOM | 704 | C | GLY A | 197 | 66.466 | 15.066 | 102.223 | 1.00 | 25.89 | A | C |
| ATOM | 705 | O | GLY A | 197 | 65.399 | 15.077 | 102.803 | 1.00 | 26.43 | A | O |
| ATOM | 706 | N | ILE A | 198 | 67.548 | 15.648 | 102.719 | 1.00 | 24.59 | A | N |
| ATOM | 707 | CA | ILE A | 198 | 67.515 | 16.336 | 104.000 | 1.00 | 27.36 | A | C |
| ATOM | 708 | CB | ILE A | 198 | 68.929 | 16.809 | 104.385 | 1.00 | 29.59 | A | C |
| ATOM | 709 | CG2 | ILE A | 198 | 69.376 | 17.929 | 103.447 | 1.00 | 29.40 | A | C |
| ATOM | 710 | CG1 | ILE A | 198 | 68.946 | 17.268 | 105.840 | 1.00 | 30.60 | A | C |
| ATOM | 711 | CD1 | ILE A | 198 | 70.323 | 17.634 | 106.334 | 1.00 | 33.20 | A | C |
| ATOM | 712 | C | ILE A | 198 | 66.558 | 17.537 | 103.966 | 1.00 | 29.10 | A | C |
| ATOM | 713 | O | ILE A | 198 | 66.139 | 18.048 | 105.009 | 1.00 | 28.33 | A | O |
| ATOM | 714 | N | GLN A | 199 | 66.212 | 17.975 | 102.760 | 1.00 | 27.92 | A | N |
| ATOM | 715 | CA | GLN A | 199 | 65.304 | 19.100 | 102.578 | 1.00 | 28.08 | A | C |
| ATOM | 716 | CB | GLN A | 199 | 65.103 | 19.366 | 101.081 | 1.00 | 31.22 | A | C |
| ATOM | 717 | CG | GLN A | 199 | 65.534 | 20.754 | 100.637 | 1.00 | 40.25 | A | C |
| ATOM | 718 | CD | GLN A | 199 | 66.996 | 21.044 | 100.939 | 1.00 | 42.74 | A | C |
| ATOM | 719 | OE1 | GLN A | 199 | 67.459 | 22.173 | 100.787 | 1.00 | 45.88 | A | O |
| ATOM | 720 | NE2 | GLN A | 199 | 67.730 | 20.022 | 101.362 | 1.00 | 44.96 | A | N |
| ATOM | 721 | C | GLN A | 199 | 63.967 | 18.794 | 103.241 | 1.00 | 23.32 | A | C |
| ATOM | 722 | O | GLN A | 199 | 63.442 | 17.705 | 103.097 | 1.00 | 20.60 | A | O |
| ATOM | 723 | N | GLY A | 200 | 63.416 | 19.759 | 103.967 | 1.00 | 23.07 | A | N |
| ATOM | 724 | CA | GLY A | 200 | 62.147 | 19.537 | 104.637 | 1.00 | 19.23 | A | C |
| ATOM | 725 | C | GLY A | 200 | 62.279 | 18.891 | 106.009 | 1.00 | 18.68 | A | C |
| ATOM | 726 | O | GLY A | 200 | 61.283 | 18.719 | 106.708 | 1.00 | 22.62 | A | O |
| ATOM | 727 | N | ASP A | 201 | 63.496 | 18.532 | 106.405 | 1.00 | 17.22 | A | N |
| ATOM | 728 | CA | ASP A | 201 | 63.712 | 17.903 | 107.711 | 1.00 | 18.97 | A | C |
| ATOM | 729 | CB | ASP A | 201 | 64.974 | 17.038 | 107.687 | 1.00 | 16.29 | A | C |
| ATOM | 730 | CG | ASP A | 201 | 64.834 | 15.835 | 106.774 | 1.00 | 18.51 | A | C |
| ATOM | 731 | OD1 | ASP A | 201 | 63.724 | 15.612 | 106.260 | 1.00 | 15.25 | A | O |
| ATOM | 732 | OD2 | ASP A | 201 | 65.824 | 15.104 | 106.576 | 1.00 | 19.34 | A | O |
| ATOM | 733 | C | ASP A | 201 | 63.813 | 18.920 | 108.844 | 1.00 | 19.43 | A | C |
| ATOM | 734 | O | ASP A | 201 | 64.156 | 20.076 | 108.621 | 1.00 | 21.36 | A | O |
| ATOM | 735 | N | ALA A | 202 | 63.513 | 18.476 | 110.062 | 1.00 | 19.70 | A | N |
| ATOM | 736 | CA | ALA A | 202 | 63.559 | 19.341 | 111.239 | 1.00 | 18.01 | A | C |
| ATOM | 737 | CB | ALA A | 202 | 62.142 | 19.616 | 111.729 | 1.00 | 17.65 | A | C |
| ATOM | 738 | C | ALA A | 202 | 64.389 | 18.706 | 112.350 | 1.00 | 18.31 | A | C |
| ATOM | 739 | O | ALA A | 202 | 64.107 | 17.588 | 112.790 | 1.00 | 17.93 | A | O |
| ATOM | 740 | N | HIS A | 203 | 65.407 | 19.431 | 112.805 | 1.00 | 17.90 | A | N |
| ATOM | 741 | CA | HIS A | 203 | 66.304 | 18.945 | 113.847 | 1.00 | 17.65 | A | C |
| ATOM | 742 | CB | HIS A | 203 | 67.736 | 18.938 | 113.313 | 1.00 | 15.54 | A | C |
| ATOM | 743 | CG | HIS A | 203 | 67.906 | 18.147 | 112.053 | 1.00 | 16.78 | A | C |
| ATOM | 744 | CD2 | HIS A | 203 | 67.100 | 17.239 | 111.453 | 1.00 | 14.93 | A | C |
| ATOM | 745 | ND1 | HIS A | 203 | 69.025 | 18.248 | 111.258 | 1.00 | 15.86 | A | N |
| ATOM | 746 | CE1 | HIS A | 203 | 68.902 | 17.439 | 110.221 | 1.00 | 16.22 | A | C |
| ATOM | 747 | NE2 | HIS A | 203 | 67.742 | 16.816 | 110.316 | 1.00 | 14.71 | A | N |
| ATOM | 748 | C | HIS A | 203 | 66.241 | 19.782 | 115.123 | 1.00 | 17.94 | A | C |
| ATOM | 749 | O | HIS A | 203 | 66.248 | 21.008 | 115.063 | 1.00 | 17.49 | A | O |
| ATOM | 750 | N | PHE A | 204 | 66.193 | 19.115 | 116.275 | 1.00 | 17.60 | A | N |
| ATOM | 751 | CA | PHE A | 204 | 66.133 | 19.814 | 117.559 | 1.00 | 17.27 | A | C |
| ATOM | 752 | CB | PHE A | 204 | 64.884 | 19.389 | 118.342 | 1.00 | 15.27 | A | C |
| ATOM | 753 | CG | PHE A | 204 | 63.602 | 19.576 | 117.579 | 1.00 | 15.59 | A | C |
| ATOM | 754 | CD1 | PHE A | 204 | 63.234 | 18.678 | 116.581 | 1.00 | 15.30 | A | C |
| ATQM | 755 | CD2 | PHE A | 204 | 62.790 | 20.683 | 117.816 | 1.00 | 15.08 | A | C |
| ATOM | 756 | CE1 | PHE A | 204 | 62.075 | 18.882 | 115.826 | 1.00 | 17.01 | A | C |
| ATOM | 757 | CE2 | PHE A | 204 | 61.636 | 20.899 | 117.071 | 1.00 | 14.70 | A | C |
| ATOM | 758 | CZ | PHE A | 204 | 61.275 | 19.998 | 116.073 | 1.00 | 16.71 | A | C |
| ATOM | 759 | C | PHE A | 204 | 67.378 | 19.555 | 118.402 | 1.00 | 17.45 | A | C |
| ATOM | 760 | O | PHE A | 204 | 67.834 | 18.414 | 118.510 | 1.00 | 15.59 | A | O |
| ATOM | 761 | N | ASP A | 205 | 67.928 | 20.619 | 118.987 | 1.00 | 16.31 | A | N |
| ATOM | 762 | CA | ASP A | 205 | 69.109 | 20.503 | 119.837 | 1.00 | 16.90 | A | C |
| ATOM | 763 | CB | ASP A | 205 | 69.729 | 21.876 | 120.082 | 1.00 | 17.51 | A | C |
| ATOM | 764 | CG | ASP A | 205 | 71.037 | 21.788 | 120.840 | 1.00 | 18.97 | A | C |
| ATOM | 765 | OD1 | ASP A | 205 | 71.305 | 20.732 | 121.441 | 1.00 | 18.91 | A | O |
| ATOM | 766 | OD2 | ASP A | 205 | 71.798 | 22.767 | 120.836 | 1.00 | 18.05 | A | O |
| ATOM | 767 | C | ASP A | 205 | 68.730 | 19.883 | 121.185 | 1.00 | 17.81 | A | C |
| ATOM | 768 | O | ASP A | 205 | 68.024 | 20.498 | 121.988 | 1.00 | 18.19 | A | O |
| ATOM | 769 | N | ASP A | 206 | 69.208 | 18.670 | 121.436 | 1.00 | 19.02 | A | N |
| ATOM | 770 | CA | ASP A | 206 | 68.880 | 17.994 | 122.676 | 1.00 | 20.78 | A | C |
| ATOM | 771 | CB | ASP A | 206 | 68.881 | 16.473 | 122.468 | 1.00 | 22.45 | A | C |
| ATOM | 772 | CG | ASP A | 206 | 68.015 | 15.737 | 123.497 | 1.00 | 25.21 | A | C |
| ATOM | 773 | OD1 | ASP A | 206 | 67.013 | 16.317 | 123.968 | 1.00 | 26.38 | A | O |
| ATOM | 774 | OD2 | ASP A | 206 | 68.321 | 14.574 | 123.822 | 1.00 | 24.25 | A | O |
| ATOM | 775 | C | ASP A | 206 | 69.781 | 18.386 | 123.847 | 1.00 | 21.59 | A | C |
| ATOM | 776 | O | ASP A | 206 | 69.827 | 17.692 | 124.849 | 1.00 | 23.96 | A | O |
| ATOM | 777 | N | ASP A | 207 | 70.513 | 19.488 | 123.715 | 1.00 | 23.27 | A | N |
| ATOM | 778 | CA | ASP A | 207 | 71.331 | 19.969 | 124.823 | 1.00 | 22.12 | A | C |
| ATOM | 779 | CB | ASP A | 207 | 72.605 | 20.664 | 124.342 | 1.00 | 23.08 | A | C |
| ATOM | 780 | CG | ASP A | 207 | 73.809 | 19.738 | 124.348 | 1.00 | 24.02 | A | C |
| ATOM | 781 | OD1 | ASP A | 207 | 73.806 | 18.762 | 125.124 | 1.00 | 26.09 | A | O |
| ATOM | 782 | OD2 | ASP A | 207 | 74.766 | 19.989 | 123.591 | 1.00 | 23.97 | A | O |
| ATOM | 783 | C | ASP A | 207 | 70.458 | 20.966 | 125.570 | 1.00 | 21.36 | A | C |
| ATOM | 784 | O | ASP A | 207 | 70.817 | 21.459 | 126.634 | 1.00 | 22.22 | A | O |
| ATOM | 785 | N | GLU A | 208 | 69.303 | 21.255 | 124.982 | 1.00 | 20.40 | A | N |
| ATOM | 786 | CA | GLU A | 208 | 68.341 | 22.172 | 125.567 | 1.00 | 21.00 | A | C |
| ATOM | 787 | CB | GLU A | 208 | 67.477 | 22.810 | 124.475 | 1.00 | 22.83 | A | C |
| ATOM | 788 | CG | GLU A | 208 | 68.262 | 23.464 | 123.363 | 1.00 | 27.27 | A | C |
| ATOM | 789 | CD | GLU A | 208 | 68.931 | 24.732 | 123.805 | 1.00 | 29.69 | A | C |
| ATOM | 790 | OE1 | GLU A | 208 | 69.277 | 24.846 | 124.996 | 1.00 | 36.53 | A | O |
| ATOM | 791 | OE2 | GLU A | 208 | 69.119 | 25.620 | 122.959 | 1.00 | 34.99 | A | O |
| ATOM | 792 | C | GLU A | 208 | 67.446 | 21.345 | 126.464 | 1.00 | 19.62 | A | C |
| ATOM | 793 | O | GLU A | 208 | 67.323 | 20.140 | 126.271 | 1.00 | 18.21 | A | O |
| ATOM | 794 | N | LEU A | 209 | 66.831 | 21.990 | 127.447 | 1.00 | 18.09 | A | N |
| ATOM | 795 | CA | LEU A | 209 | 65.898 | 21.311 | 128.331 | 1.00 | 17.54 | A | C |
| ATOM | 796 | CB | LEU A | 209 | 65.887 | 21.982 | 129.710 | 1.00 | 15.57 | A | C |
| ATOM | 797 | CG | LEU A | 209 | 64.850 | 21.473 | 130.714 | 1.00 | 19.72 | A | C |
| ATOM | 798 | CD1 | LEU A | 209 | 65.072 | 20.004 | 130.986 | 1.00 | 18.15 | A | C |
| ATOM | 799 | CD2 | LEU A | 209 | 64.947 | 22.273 | 132.008 | 1.00 | 18.92 | A | C |
| ATOM | 800 | C | LEU A | 209 | 64.529 | 21.445 | 127.653 | 1.00 | 16.85 | A | C |
| ATOM | 801 | O | LEU A | 209 | 64.001 | 22.549 | 127.519 | 1.00 | 18.96 | A | O |
| ATOM | 802 | N | TRP A | 210 | 63.974 | 20.328 | 127.197 | 1.00 | 15.69 | A | N |
| ATOM | 803 | CA | TRP A | 210 | 62.678 | 20.347 | 126.531 | 1.00 | 14.68 | A | C |
| ATOM | 804 | CB | TRP A | 210 | 62.628 | 19.319 | 125.384 | 1.00 | 12.69 | A | C |
| ATOM | 805 | CG | TRP A | 210 | 63.520 | 19.686 | 124.252 | 1.00 | 14.19 | A | C |
| ATOM | 806 | CD2 | TRP A | 210 | 63.396 | 20.834 | 123.402 | 1.00 | 14.74 | A | C |
| ATOM | 807 | CE2 | TRP A | 210 | 64.531 | 20.855 | 122.562 | 1.00 | 13.82 | A | C |
| ATOM | 808 | CE3 | TRP A | 210 | 62.438 | 21.850 | 123.273 | 1.00 | 13.00 | A | C |
| ATOM | 809 | CD1 | TRP A | 210 | 64.688 | 19.072 | 123.893 | 1.00 | 15.08 | A | C |
| ATOM | 810 | NE1 | TRP A | 210 | 65.302 | 19.770 | 122.885 | 1.00 | 15.30 | A | N |
| ATOM | 811 | CZ2 | TRP A | 210 | 64.736 | 21.854 | 121.605 | 1.00 | 12.58 | A | C |
| ATOM | 812 | CZ3 | TRP A | 210 | 62.646 | 22.845 | 122.319 | 1.00 | 11.89 | A | C |
| ATOM | 813 | CH2 | TRP A | 210 | 63.787 | 22.837 | 121.499 | 1.00 | 10.54 | A | C |
| ATOM | 814 | C | TRP A | 210 | 61.549 | 20.080 | 127.503 | 1.00 | 14.10 | A | C |
| ATOM | 815 | O | TRP A | 210 | 61.614 | 19.147 | 128.301 | 1.00 | 12.40 | A | O |
| ATOM | 816 | N | SER A | 211 | 60.504 | 20.897 | 127.406 | 1.00 | 13.94 | A | N |
| ATOM | 817 | CA | SER A | 211 | 59.347 | 20.791 | 128.272 | 1.00 | 13.15 | A | C |
| ATOM | 818 | CB | SER A | 211 | 59.568 | 21.682 | 129.492 | 1.00 | 14.15 | A | C |
| ATOM | 819 | OG | SER A | 211 | 58.635 | 21.392 | 130.505 | 1.00 | 17.46 | A | O |
| ATOM | 820 | C | SER A | 211 | 58.102 | 21.243 | 127.505 | 1.00 | 15.84 | A | C |
| ATOM | 821 | O | SER A | 211 | 58.080 | 21.195 | 126.280 | 1.00 | 17.08 | A | O |
| ATOM | 822 | N | LEU A | 212 | 57.068 | 21.667 | 128.233 | 1.00 | 15.21 | A | N |
| ATOM | 823 | CA | LEU A | 212 | 55.819 | 22.160 | 127.645 | 1.00 | 13.96 | A | C |
| ATOM | 824 | CB | LEU A | 212 | 54.836 | 21.018 | 127.391 | 1.00 | 10.99 | A | C |
| ATOM | 825 | CG | LEU A | 212 | 55.168 | 19.921 | 126.378 | 1.00 | 11.66 | A | C |
| ATOM | 826 | CD1 | LEU A | 212 | 54.139 | 18.817 | 126.510 | 1.00 | 8.67 | A | C |
| ATOM | 827 | CD2 | LEU A | 212 | 55.202 | 20.494 | 124.967 | 1.00 | 7.27 | A | U |
| ATOM | 828 | C | LEU A | 212 | 55.180 | 23.125 | 128.635 | 1.00 | 17.00 | A | C |
| ATOM | 829 | O | LEU A | 212 | 55.367 | 22.992 | 129.837 | 1.00 | 19.75 | A | O |
| ATOM | 830 | N | GLY A | 213 | 54.421 | 24.088 | 128.132 | 1.00 | 19.26 | A | N |
| ATOM | 831 | CA | GLY A | 213 | 53.748 | 25.037 | 129.003 | 1.00 | 20.32 | A | C |
| ATOM | 832 | C | GLY A | 213 | 54.639 | 26.040 | 129.713 | 1.00 | 19.73 | A | C |
| ATOM | 833 | O | GLY A | 213 | 55.759 | 26.294 | 129.285 | 1.00 | 20.34 | A | O |
| ATOM | 834 | N | LYS A | 214 | 54.123 | 26.618 | 130.795 | 1.00 | 20.81 | A | N |
| ATOM | 835 | CA | LYS A | 214 | 54.861 | 27.597 | 131.588 | 1.00 | 21.44 | A | C |
| ATOM | 836 | CB | LYS A | 214 | 53.909 | 28.339 | 132.537 | 1.00 | 23.26 | A | C |
| ATOM | 837 | CG | LYS A | 214 | 52.925 | 29.266 | 13 1.838 | 1.00 | 25.72 | A | C |
| ATOM | 838 | CD | LYS A | 214 | 51.935 | 29.898 | 132.805 | 1.00 | 27.35 | A | C |
| ATOM | 839 | CE | LYS A | 214 | 51.002 | 28.859 | 133.402 | 1.00 | 30.75 | A | C |
| ATOM | 840 | NZ | LYS A | 214 | 49.866 | 29.485 | 134.138 | 1.00 | 35.19 | A | N |
| ATOM | 841 | C | LYS A | 214 | 55.917 | 26.863 | 132.398 | 1.00 | 21.38 | A | C |
| ATOM | 842 | O | LYS A | 214 | 55.743 | 25.688 | 132.729 | 1.00 | 22.09 | A | O |
| ATOM | 843 | N | GLY A | 215 | 57.008 | 27.548 | 132.721 | 1.00 | 19.58 | A | N |
| ATOM | 844 | CA | GLY A | 215 | 58.061 | 26.911 | 133.491 | 1.00 | 20.65 | A | C |
| ATOM | 845 | C | GLY A | 215 | 59.403 | 27.007 | 132.798 | 1.00 | 20.80 | A | C |
| ATOM | 846 | O | GLY A | 215 | 59.496 | 27.528 | 131.689 | 1.00 | 20.47 | A | O |
| ATOM | 847 | N | GLN A | 391 | 60.449 | 26.500 | 133.439 | 1.00 | 21.31 | A | N |
| ATOM | 848 | CA | GLN A | 391 | 61.779 | 26.564 | 132.848 | 1.00 | 22.25 | A | C |
| ATOM | 849 | CB | GLN A | 391 | 62.851 | 26.279 | 133.917 | 1.00 | 26.72 | A | C |
| ATOM | 850 | CG | GLN A | 391 | 63.213 | 24.832 | 134.110 | 1.00 | 34.22 | A | C |
| ATOM | 851 | CD | GLN A | 391 | 62.000 | 23.953 | 134.238 | 1.00 | 41.18 | A | C |
| ATOM | 852 | OE1 | GLN A | 391 | 61.117 | 24.211 | 135.054 | 1.00 | 48.75 | A | O |
| ATOM | 853 | NE2 | GLN A | 391 | 61.944 | 22.903 | 133.427 | 1.00 | 46.74 | A | N |
| ATOM | 854 | C | GLN A | 391 | 61.886 | 25.585 | 131.676 | 1.00 | 20.85 | A | C |
| ATOM | 855 | O | GLN A | 391 | 61.097 | 24.654 | 131.561 | 1.00 | 20.10 | A | O |
| ATOM | 856 | N | GLY A | 392 | 62.866 | 25.794 | 130.808 | 1.00 | 19.12 | A | N |
| ATOM | 857 | CA | GLY A | 392 | 63.005 | 24.931 | 129.653 | 1.00 | 20.95 | A | C |
| ATOM | 858 | C | GLY A | 392 | 62.266 | 25.565 | 128.487 | 1.00 | 19.62 | A | C |
| ATOM | 859 | O | GLY A | 392 | 61.669 | 26.627 | 128.637 | 1.00 | 17.25 | A | O |
| ATOM | 860 | N | TYR A | 393 | 62.301 | 24.921 | 127.328 | 1.00 | 19.08 | A | N |
| ATOM | 861 | CA | TYR A | 393 | 61.637 | 25.454 | 126.141 | 1.00 | 17.64 | A | C |
| ATOM | 862 | CB | TYR A | 393 | 62.646 | 25.560 | 125.000 | 1.00 | 18.65 | A | C |
| ATOM | 863 | CG | TYR A | 393 | 63.773 | 26.534 | 125.256 | 1.00 | 18.22 | A | C |
| ATOM | 864 | CD1 | TYR A | 393 | 63.586 | 27.905 | 125.079 | 1.00 | 19.12 | A | C |
| ATOM | 865 | CE1 | TYR A | 393 | 64.626 | 28.807 | 125.291 | 1.00 | 20.27 | A | C |
| ATOM | 866 | CD2 | TYR A | 393 | 65.036 | 26.084 | 125.660 | 1.00 | 18.43 | A | C |
| ATOM | 867 | CE2 | TYR A | 393 | 66.091 | 26.980 | 125.878 | 1.00 | 18.43 | A | C |
| ATOM | 868 | CZ | TYR A | 393 | 65.879 | 28.339 | 125.691 | 1.00 | 20.99 | A | C |
| ATOM | 869 | OH | TYR A | 393 | 66.911 | 29.229 | 125.892 | 1.00 | 18.81 | A | O |
| ATOM | 870 | C | TYR A | 393 | 60.474 | 24.563 | 125.714 | 1.00 | 16.46 | A | C |
| ATOM | 871 | O | TYR A | 393 | 60.552 | 23.343 | 125.812 | 1.00 | 18.48 | A | O |
| ATOM | 872 | N | SER A | 394 | 59.393 | 25.165 | 125.238 | 1.00 | 16.19 | A | N |
| ATOM | 873 | CA | SER A | 394 | 58.248 | 24.376 | 124.799 | 1.00 | 16.88 | A | C |
| ATOM | 874 | CB | SER A | 394 | 57.026 | 25.268 | 124.581 | 1.00 | 15.95 | A | C |
| ATOM | 875 | OG | SER A | 394 | 55.900 | 24.491 | 124.195 | 1.00 | 16.81 | A | O |
| ATOM | 876 | C | SER A | 394 | 58.555 | 23.638 | 123.501 | 1.00 | 16.05 | A | C |
| ATOM | 877 | O | SER A | 394 | 58.781 | 24.262 | 122.468 | 1.00 | 17.59 | A | O |
| ATOM | 878 | N | LEU A | 395 | 58.563 | 22.311 | 123.557 | 1.00 | 13.28 | A | N |
| ATOM | 879 | CA | LEU A | 395 | 58.825 | 21.508 | 122.373 | 1.00 | 13.52 | A | C |
| ATOM | 880 | CB | LEU A | 395 | 58.874 | 20.022 | 122.734 | 1.00 | 9.81 | A | C |
| ATOM | 881 | CG | LEU A | 395 | 59.158 | 19.078 | 121.559 | 1.00 | 10.42 | A | C |
| ATOM | 882 | CD1 | LEU A | 395 | 60.518 | 19.417 | 120.974 | 1.00 | 8.66 | A | C |
| ATOM | 883 | CD2 | LEU A | 395 | 59.117 | 17.618 | 122.012 | 1.00 | 7.01 | A | C |
| ATOM | 884 | C | LEU A | 395 | 57.720 | 21.758 | 121.345 | 1.00 | 13.87 | A | C |
| ATOM | 885 | O | LEU A | 395 | 57.979 | 21.792 | 120.149 | 1.00 | 15.88 | A | O |
| ATOM | 886 | N | PHE A | 396 | 56.495 | 21.936 | 121.836 | 1.00 | 13.30 | A | N |
| ATOM | 887 | CA | PHE A | 396 | 55.319 | 22.195 | 121.010 | 1.00 | 13.78 | A | C |
| ATOM | 888 | CB | PHE A | 396 | 54.069 | 22.270 | 121.907 | 1.00 | 14.03 | A | C |
| ATOM | 889 | CG | PHE A | 396 | 52.850 | 22.840 | 121.225 | 1.00 | 15.47 | A | C |
| ATOM | 890 | CD1 | PHE A | 396 | 52.243 | 22.168 | 120.167 | 1.00 | 16.74 | A | C |
| ATOM | 891 | CD2 | PHE A | 396 | 52.322 | 24.064 | 121.632 | 1.00 | 16.39 | A | C |
| ATOM | 892 | CE1 | PHE A | 396 | 51.128 | 22.706 | 119.518 | 1.00 | 15.98 | A | C |
| ATOM | 893 | CE2 | PHE A | 396 | 51.211 | 24.611 | 120.993 | 1.00 | 17.97 | A | C |
| ATOM | 894 | CZ | PHE A | 396 | 50.611 | 23.929 | 119.929 | 1.00 | 16.67 | A | C |
| ATOM | 895 | C | PHE A | 396 | 55.440 | 23.476 | 120.178 | 1.00 | 15.62 | A | C |
| ATOM | 896 | O | PHE A | 396 | 55.242 | 23.455 | 118.958 | 1.00 | 15.38 | A | O |
| ATOM | 897 | N | LEU A | 397 | 55.756 | 24.587 | 120.837 | 1.00 | 14.66 | A | N |
| ATOM | 898 | CA | LEU A | 397 | 55.892 | 25.861 | 120.148 | 1.00 | 14.57 | A | C |
| ATOM | 899 | CB | LEU A | 397 | 55.998 | 27.009 | 121.155 | 1.00 | 16.23 | A | C |
| ATOM | 900 | CG | LEU A | 397 | 54.739 | 27.366 | 121.951 | 1.00 | 19.61 | A | C |
| ATOM | 901 | CD1 | LEU A | 397 | 55.033 | 28.532 | 122.908 | 1.00 | 17.04 | A | C |
| ATOM | 902 | CD2 | LEU A | 397 | 53.615 | 27.734 | 120.982 | 1.00 | 18.70 | A | C |
| ATOM | 903 | C | LEU A | 397 | 57.084 | 25.912 | 119.202 | 1.00 | 16.31 | A | C |
| ATOM | 904 | O | LEU A | 397 | 57.002 | 26.511 | 118.132 | 1.00 | 18.67 | A | O |
| ATOM | 905 | N | VAL A | 398 | 58.198 | 25.299 | 119.591 | 1.00 | 17.18 | A | N |
| ATOM | 906 | CA | VAL A | 398 | 59.379 | 25.309 | 118.738 | 1.00 | 16.87 | A | C |
| ATOM | 907 | CB | VAL A | 398 | 60.643 | 24.855 | 119.513 | 1.00 | 15.03 | A | C |
| ATOM | 908 | CG1 | VAL A | 398 | 61.838 | 24.750 | 118.562 | 1.00 | 7.55 | A | C |
| ATOM | 909 | CG2 | VAL A | 398 | 60.953 | 25.867 | 120.636 | 1.00 | 13.62 | A | C |
| ATOM | 910 | C | VAL A | 398 | 59.157 | 24.410 | 117.524 | 1.00 | 17.35 | A | C |
| ATOM | 911 | O | VAL A | 398 | 59.610 | 24.715 | 116.427 | 1.00 | 16.12 | A | O |
| ATOM | 912 | N | ALA A | 399 | 58.446 | 23.307 | 117.725 | 1.00 | 17.18 | A | N |
| ATOM | 913 | CA | ALA A | 399 | 58.159 | 22.386 | 116.629 | 1.00 | 18.10 | A | C |
| ATOM | 914 | CB | ALA A | 399 | 57.440 | 21.146 | 117.155 | 1.00 | 17.14 | A | C |
| ATOM | 915 | C | ALA A | 399 | 57.296 | 23.084 | 115.583 | 1.00 | 16.31 | A | C |
| ATOM | 916 | O | ALA A | 399 | 57.540 | 22.962 | 114.387 | 1.00 | 17.90 | A | O |
| ATOM | 917 | N | ALA A | 400 | 56.288 | 23.814 | 116.048 | 1.00 | 16.32 | A | N |
| ATOM | 918 | CA | ALA A | 400 | 55.385 | 24.531 | 115.161 | 1.00 | 16.53 | A | C |
| ATOM | 919 | CB | ALA A | 400 | 54.340 | 25.283 | 115.970 | 1.00 | 11.88 | A | C |
| ATOM | 920 | C | ALA A | 400 | 56.162 | 25.495 | 114.278 | 1.00 | 17.89 | A | C |
| ATOM | 921 | O | ALA A | 400 | 55.859 | 25.640 | 113.101 | 1.00 | 18.21 | A | O |
| ATOM | 922 | N | HIS A | 401 | 57.169 | 26.142 | 114.858 | 1.00 | 18.94 | A | N |
| ATOM | 923 | CA | HIS A | 401 | 58.009 | 27.094 | 114.140 | 1.00 | 16.00 | A | C |
| ATOM | 924 | CB | HIS A | 401 | 58.934 | 27.812 | 115.123 | 1.00 | 15.08 | A | C |
| ATOM | 925 | CG | HIS A | 401 | 59.800 | 28.858 | 114.490 | 1.00 | 16.72 | A | C |
| ATOM | 926 | CD2 | HIS A | 401 | 61.049 | 28.788 | 113.972 | 1.00 | 11.82 | A | C |
| ATOM | 927 | ND1 | HIS A | 401 | 59.400 | 30.169 | 114.344 | 1.00 | 16.08 | A | N |
| ATOM | 928 | CE1 | HIS A | 401 | 60.367 | 30.860 | 113.767 | 1.00 | 16.27 | A | C |
| ATOM | 929 | NE2 | HIS A | 401 | 61.377 | 30.045 | 113.531 | 1.00 | 10.47 | A | N |
| ATOM | 930 | C | HIS A | 401 | 58.849 | 26.371 | 113.091 | 1.00 | 17.48 | A | C |
| ATOM | 931 | O | HIS A | 401 | 58.899 | 26.785 | 111.939 | 1.00 | 17.04 | A | O |
| ATOM | 932 | N | GLU A | 402 | 59.512 | 25.292 | 113.497 | 1.00 | 16.98 | A | N |
| ATOM | 933 | CA | GLU A | 402 | 60.346 | 24.523 | 112.583 | 1.00 | 19.94 | A | C |
| ATOM | 934 | CB | GLU A | 402 | 61.058 | 23.374 | 113.311 | 1.00 | 20.34 | A | C |
| ATOM | 935 | CG | GLU A | 402 | 62.029 | 23.804 | 114.410 | 1.00 | 20.68 | A | C |
| ATOM | 936 | CD | GLU A | 402 | 62.758 | 25.100 | 114.095 | 1.00 | 17.45 | A | C |
| ATOM | 937 | OE1 | GLU A | 402 | 63.300 | 25.234 | 112.987 | 1.00 | 18.70 | A | O |
| ATOM | 938 | OE2 | GLU A | 402 | 62.793 | 25.984 | 114.967 | 1.00 | 17.38 | A | O |
| ATOM | 939 | C | GLU A | 402 | 59.522 | 23.952 | 111.432 | 1.00 | 20.70 | A | C |
| ATOM | 940 | O | GLU A | 402 | 59.961 | 23.972 | 110.285 | 1.00 | 20.70 | A | O |
| ATOM | 941 | N | PHE A | 403 | 58.336 | 23.437 | 111.735 | 1.00 | 20.02 | A | N |
| ATOM | 942 | CA | PHE A | 403 | 57.482 | 22.889 | 110.691 | 1.00 | 20.64 | A | C |
| ATOM | 943 | CB | PHE A | 403 | 56.262 | 22.184 | 111.293 | 1.00 | 20.51 | A | C |
| ATOM | 944 | CG | PHE A | 403 | 56.608 | 21.005 | 112.173 | 1.00 | 20.43 | A | C |
| ATOM | 945 | CD1 | PHE A | 403 | 57.865 | 20.398 | 112.094 | 1.00 | 17.64 | A | C |
| ATOM | 946 | CD2 | PHE A | 403 | 55.678 | 20.509 | 113.087 | 1.00 | 14.31 | A | C |
| ATOM | 947 | CE1 | PHE A | 403 | 58.192 | 19.319 | 112.916 | 1.00 | 18.71 | A | C |
| ATOM | 948 | CE2 | PHE A | 403 | 55.988 | 19.424 | 113.918 | 1.00 | 12.75 | A | C |
| ATOM | 949 | CZ | PHE A | 403 | 57.243 | 18.826 | 113.838 | 1.00 | 15.90 | A | C |
| ATOM | 950 | C | PHE A | 403 | 57.040 | 23.998 | 109.732 | 1.00 | 20.93 | A | C |
| ATOM | 951 | O | PHE A | 403 | 56.699 | 23.728 | 108.587 | 1.00 | 20.08 | A | O |
| ATOM | 952 | N | GLY A | 404 | 57.041 | 25.241 | 110.208 | 1.00 | 20.24 | A | N |
| ATOM | 953 | CA | GLY A | 404 | 56.685 | 26.355 | 109.352 | 1.00 | 18.45 | A | C |
| ATOM | 954 | C | GLY A | 404 | 57.748 | 26.442 | 108.263 | 1.00 | 19.06 | A | C |
| ATOM | 955 | O | GLY A | 404 | 57.438 | 26.584 | 107.082 | 1.00 | 18.85 | A | O |
| ATOM | 956 | N | HIS A | 405 | 59.011 | 26.353 | 108.666 | 1.00 | 17.32 | A | N |
| ATOM | 957 | CA | HIS A | 405 | 60.119 | 26.382 | 107.718 | 1.00 | 18.09 | A | C |
| ATOM | 958 | CB | HIS A | 405 | 61.460 | 26.317 | 108.446 | 1.00 | 15.13 | A | C |
| ATOM | 959 | CG | HIS A | 405 | 61.859 | 27.600 | 109.095 | 1.00 | 19.30 | A | C |
| ATOM | 960 | CD2 | HIS A | 405 | 62.362 | 27.859 | 110.323 | 1.00 | 15.40 | A | C |
| ATOM | 961 | ND1 | HIS A | 405 | 61.769 | 28.816 | 108.452 | 1.00 | 18.08 | A | N |
| ATOM | 962 | CE1 | HIS A | 405 | 62.197 | 29.769 | 109.258 | 1.00 | 17.34 | A | C |
| ATOM | 963 | NE2 | HIS A | 405 | 62.563 | 29.214 | 110.399 | 1.00 | 18.40 | A | N |
| ATOM | 964 | C | HIS A | 405 | 60.040 | 25.195 | 106.761 | 1.00 | 18.79 | A | C |
| ATOM | 965 | O | HIS A | 405 | 60.171 | 25.352 | 105.554 | 1.00 | 22.20 | A | O |
| ATOM | 966 | N | ALA A | 406 | 59.838 | 24.007 | 107.320 | 1.00 | 18.37 | A | N |
| ATOM | 967 | CA | ALA A | 406 | 59.754 | 22.780 | 106.545 | 1.00 | 17.99 | A | C |
| ATOM | 968 | CB | ALA A | 406 | 59.511 | 21.606 | 107.484 | 1.00 | 15.77 | A | C |
| ATOM | 969 | C | ALA A | 406 | 58.665 | 22.838 | 105.468 | 1.00 | 17.88 | A | C |
| ATOM | 970 | O | ALA A | 406 | 58.673 | 22.045 | 104.535 | 1.00 | 15.94 | A | O |
| ATOM | 971 | N | LEU A | 407 | 57.730 | 23.173 | 105.612 | 1.00 | 20.32 | A | N |
| ATOM | 972 | CA | LEU A | 407 | 56.646 | 23.935 | 104.647 | 1.00 | 22.45 | A | C |
| ATOM | 973 | CB | LEU A | 407 | 55.307 | 24.158 | 105.367 | 1.00 | 21.52 | A | C |
| ATOM | 974 | CG | LEU A | 407 | 54.789 | 23.030 | 106.266 | 1.00 | 22.77 | A | C |
| ATOM | 975 | CD1 | LEU A | 407 | 53.461 | 23.421 | 106.888 | 1.00 | 19.54 | A | C |
| ATOM | 976 | CD2 | LEU A | 407 | 54.629 | 21.766 | 105.447 | 1.00 | 25.90 | A | C |
| ATOM | 977 | C | LEU A | 407 | 56.921 | 25.103 | 103.694 | 1.00 | 23.47 | A | C |
| ATOM | 978 | O | LEU A | 407 | 56.093 | 25.422 | 102.844 | 1.00 | 23.57 | A | O |
| ATOM | 979 | N | GLY A | 408 | 58.077 | 25.743 | 103.852 | 1.00 | 22.90 | A | N |
| ATOM | 980 | CA | GLY A | 408 | 58.444 | 26.847 | 102.983 | 1.00 | 23.36 | A | C |
| ATOM | 981 | C | GLY A | 408 | 58.411 | 28.268 | 103.528 | 1.00 | 24.25 | A | C |
| ATOM | 982 | O | GLY A | 408 | 58.891 | 29.190 | 102.868 | 1.00 | 22.84 | A | O |
| ATOM | 983 | N | LEU A | 409 | 57.855 | 28.463 | 104.719 | 1.00 | 25.11 | A | N |
| ATOM | 984 | CA | LEU A | 409 | 57.777 | 29.804 | 105.290 | 1.00 | 24.70 | A | C |
| ATOM | 985 | CB | LEU A | 409 | 56.853 | 29.828 | 106.508 | 1.00 | 22.64 | A | C |
| ATOM | 986 | CG | LEU A | 409 | 55.352 | 29.682 | 106.281 | 1.00 | 25.21 | A | C |
| ATOM | 987 | CD1 | LEU A | 409 | 54.639 | 29.943 | 107.588 | 1.00 | 21.69 | A | C |
| ATOM | 988 | CD2 | LEU A | 409 | 54.880 | 30.664 | 105.212 | 1.00 | 24.00 | A | C |
| ATOM | 989 | C | LEU A | 409 | 59.123 | 30.347 | 105.711 | 1.00 | 25.87 | A | C |
| ATOM | 990 | O | LEU A | 409 | 59.989 | 29.598 | 106.156 | 1.00 | 26.76 | A | O |
| ATOM | 991 | N | ASP A | 410 | 59.294 | 31.656 | 105.558 | 1.00 | 27.29 | A | N |
| ATOM | 992 | CA | ASP A | 410 | 60.517 | 32.323 | 105.980 | 1.00 | 29.12 | A | C |
| ATOM | 993 | CB | ASP A | 410 | 60.990 | 33.338 | 104.933 | 1.00 | 34.79 | A | C |
| ATOM | 994 | CG | ASP A | 410 | 61.553 | 32.678 | 103.687 | 1.00 | 42.97 | A | C |
| ATOM | 995 | OD1 | ASP A | 410 | 62.184 | 31.602 | 103.799 | 1.00 | 48.11 | A | O |
| ATOM | 996 | OD2 | ASP A | 410 | 61.381 | 33.247 | 102.590 | 1.00 | 47.38 | A | O |
| ATOM | 997 | C | ASP A | 410 | 60.188 | 33.050 | 107.289 | 1.00 | 27.33 | A | C |
| ATOM | 998 | O | ASP A | 410 | 59.046 | 33.026 | 107.751 | 1.00 | 19.85 | A | O |
| ATOM | 999 | N | HIS A | 411 | 61.190 | 33.687 | 107.883 | 1.00 | 25.92 | A | N |
| ATOM | 1000 | CA | HIS A | 411 | 60.995 | 34.418 | 109.128 | 1.00 | 27.93 | A | C |
| ATOM | 1001 | CB | HIS A | 411 | 62.345 | 34.843 | 109.699 | 1.00 | 25.18 | A | C |
| ATOM | 1002 | CG | HIS A | 411 | 63.056 | 33.747 | 110.430 | 1.00 | 25.67 | A | C |
| ATOM | 1003 | CD2 | HIS A | 411 | 62.579 | 32.692 | 111.134 | 1.00 | 22.64 | A | C |
| ATOM | 1004 | ND1 | HIS A | 411 | 64.429 | 33.660 | 110.493 | 1.00 | 24.52 | A | N |
| ATOM | 1005 | CE1 | HIS A | 411 | 64.768 | 32.596 | 111.199 | 1.00 | 24.55 | A | C |
| ATOM | 1006 | NE2 | HIS A | 411 | 63.664 | 31.992 | 111.598 | 1.00 | 21.02 | A | N |
| ATOM | 1007 | C | HIS A | 411 | 60.103 | 35.634 | 108.947 | 1.00 | 28.39 | A | C |
| ATOM | 1008 | O | HIS A | 411 | 60.096 | 36.257 | 107.890 | 1.00 | 27.54 | A | O |
| ATOM | 1009 | N | SER A | 412 | 59.348 | 35.953 | 109.992 | 1.00 | 28.70 | A | N |
| ATOM | 1010 | CA | SER A | 412 | 58.441 | 37.090 | 109.989 | 1.00 | 28.94 | A | C |
| ATOM | 1011 | CE | SER A | 412 | 57.139 | 36.716 | 110.696 | 1.00 | 27.79 | A | C |
| ATOM | 1012 | OG | SER A | 412 | 56.288 | 37.836 | 110.831 | 1.00 | 28.82 | A | O |
| ATOM | 1013 | C | SER A | 412 | 59.090 | 38.264 | 110.708 | 1.00 | 30.69 | A | C |
| ATOM | 1014 | O | SER A | 412 | 60.020 | 38.081 | 111.489 | 1.00 | 31.96 | A | O |
| ATOM | 1015 | N | SER A | 413 | 58.601 | 39.468 | 110.437 | 1.00 | 30.98 | A | N |
| ATOM | 1016 | CA | SER A | 413 | 59.132 | 40.664 | 111.075 | 1.00 | 33.39 | A | C |
| ATOM | 1017 | CB | SER A | 413 | 59.334 | 41.775 | 110.039 | 1.00 | 34.31 | A | C |
| ATOM | 1018 | OG | SER A | 413 | 58.154 | 42.003 | 109.294 | 1.00 | 35.39 | A | O |
| ATOM | 1019 | C | SER A | 413 | 58.175 | 41.125 | 112.166 | 1.00 | 32.91 | A | C |
| ATOM | 1020 | O | SER A | 413 | 58.454 | 42.076 | 112.893 | 1.00 | 35.58 | A | O |
| ATOM | 1021 | N | VAL A | 414 | 57.043 | 40.436 | 112.264 | 1.00 | 31.58 | A | N |
| ATOM | 1022 | CA | VAL A | 414 | 56.023 | 40.725 | 113.264 | 1.00 | 31.56 | A | C |
| ATOM | 1023 | CB | VAL A | 414 | 54.637 | 40.234 | 112.792 | 1.00 | 31.76 | A | C |
| ATOM | 1024 | CG1 | VAL A | 414 | 53.578 | 40.582 | 113.816 | 1.00 | 31.52 | A | C |
| ATOM | 1025 | CG2 | VAL A | 414 | 54.304 | 40.849 | 111.455 | 1.00 | 30.36 | A | C |
| ATOM | 1026 | C | VAL A | 414 | 56.399 | 40.007 | 114.564 | 1.00 | 31.65 | A | C |
| ATOM | 1027 | O | VAL A | 414 | 56.415 | 38.778 | 114.630 | 1.00 | 30.34 | A | O |
| ATOM | 1028 | N | PRO A | 415 | 56.696 | 40.775 | 115.620 | 1.00 | 31.44 | A | N |
| ATOM | 1029 | CD | PRO A | 415 | 56.730 | 42.248 | 115.656 | 1.00 | 29.01 | A | C |
| ATOM | 1030 | CA | PRO A | 415 | 57.080 | 40.220 | 116.920 | 1.00 | 28.81 | A | C |
| ATOM | 1031 | CB | PRO A | 415 | 57.176 | 41.461 | 117.802 | 1.00 | 29.09 | A | C |
| ATOM | 1032 | CG | PRO A | 415 | 57.628 | 42.513 | 116.837 | 1.00 | 27.34 | A | C |
| ATOM | 1033 | C | PRO A | 415 | 56.156 | 39.157 | 117.503 | 1.00 | 26.99 | A | C |
| ATOM | 1034 | O | PRO A | 415 | 56.626 | 38.199 | 118.098 | 1.00 | 26.82 | A | O |
| ATOM | 1035 | N | GLU A | 416 | 54.849 | 39.316 | 117.336 | 1.00 | 28.55 | A | N |
| ATOM | 1036 | CA | GLU A | 416 | 53.907 | 38.343 | 117.881 | 1.00 | 29.16 | A | C |
| ATOM | 1037 | CB | GLU A | 416 | 52.597 | 39.029 | 118.276 | 1.00 | 33.01 | A | C |
| ATOM | 1038 | CG | GLU A | 416 | 52.180 | 40.147 | 117.341 | 1.00 | 42.66 | A | C |
| ATOM | 1039 | CD | GLU A | 416 | 52.947 | 41.437 | 117.590 | 1.00 | 43.72 | A | C |
| ATOM | 1040 | OE1 | GLU A | 416 | 53.148 | 42.204 | 116.626 | 1.00 | 43.99 | A | O |
| ATOM | 1041 | OE2 | GLU A | 416 | 53.336 | 41.690 | 118.752 | 1.00 | 46.02 | A | O |
| ATOM | 1042 | C | GLU A | 416 | 53.612 | 37.163 | 116.957 | 1.00 | 28.85 | A | C |
| ATOM | 1043 | O | GLU A | 416 | 52.820 | 36.289 | 117.297 | 1.00 | 30.41 | A | O |
| ATOM | 1044 | N | ALA A | 417 | 54.257 | 37.125 | 115.798 | 1.00 | 26.45 | A | N |
| ATOM | 1045 | CA | ALA A | 417 | 54.037 | 36.029 | 114.867 | 1.00 | 24.95 | A | C |
| ATOM | 1046 | CB | ALA A | 417 | 54.429 | 36.452 | 113.456 | 1.00 | 23.05 | A | C |
| ATOM | 1047 | C | ALA A | 417 | 54.868 | 34.826 | 115.303 | 1.00 | 24.52 | A | C |
| ATOM | 1048 | O | ALA A | 417 | 55.952 | 34.986 | 115.861 | 1.00 | 23.59 | A | O |
| ATOM | 1049 | N | LEU A | 418 | 54.354 | 33.625 | 115.049 | 1.00 | 22.60 | A | N |
| ATOM | 1050 | CA | LEU A | 418 | 55.060 | 32.399 | 115.396 | 1.00 | 21.25 | A | C |
| ATOM | 1051 | CB | LEU A | 418 | 54.186 | 31.183 | 115.084 | 1.00 | 24.09 | A | C |
| ATOM | 1052 | CG | LEU A | 418 | 54.819 | 29.794 | 115.255 | 1.00 | 25.61 | A | C |
| ATOM | 1053 | CD1 | LEU A | 418 | 55.205 | 29.584 | 116.704 | 1.00 | 24.01 | A | C |
| ATOM | 1054 | CD2 | LEU A | 418 | 53.851 | 28.711 | 114.802 | 1.00 | 24.22 | A | C |
| ATOM | 1055 | C | LEU A | 418 | 56.378 | 32.281 | 114.639 | 1.00 | 20.33 | A | C |
| ATOM | 1056 | O | LEU A | 418 | 57.336 | 31.699 | 115.139 | 1.00 | 17.86 | A | O |
| ATOM | 1057 | N | MET A | 419 | 56.425 | 32.836 | 113.431 | 1.00 | 20.15 | A | N |
| ATOM | 1058 | CA | MET A | 419 | 57.632 | 32.763 | 112.620 | 1.00 | 20.11 | A | C |
| ATOM | 1059 | CB | MET A | 419 | 57.286 | 32.817 | 111.130 | 1.00 | 19.41 | A | C |
| ATOM | 1060 | CG | MET A | 419 | 56.562 | 31.575 | 110.616 | 1.00 | 18.20 | A | C |
| ATOM | 1061 | SD | MET A | 419 | 57.267 | 29.973 | 111.160 | 1.00 | 18.41 | A | S |
| ATOM | 1062 | CE | MET A | 419 | 58.852 | 29.969 | 110.370 | 1.00 | 13.77 | A | C |
| ATOM | 1063 | C | MET A | 419 | 58.678 | 33.816 | 112.953 | 1.00 | 20.18 | A | C |
| ATOM | 1064 | O | MET A | 419 | 59.679 | 33.946 | 112.256 | 1.00 | 20.02 | A | O |
| ATOM | 1065 | N | TYR A | 420 | 58.450 | 34.571 | 114.022 | 1.00 | 22.13 | A | N |
| ATOM | 1066 | CA | TYR A | 420 | 59.433 | 35.561 | 114.443 | 1.00 | 23.96 | A | C |
| ATOM | 1067 | CB | TYR A | 420 | 58.921 | 36.336 | 115.653 | 1.00 | 28.74 | A | C |
| ATOM | 1068 | CG | TYR A | 420 | 59.721 | 37.578 | 115.957 | 1.00 | 33.66 | A | C |
| ATOM | 1069 | CD1 | TYR A | 420 | 59.747 | 38.654 | 115.064 | 1.00 | 35.38 | A | C |
| ATOM | 1070 | CE1 | TYR A | 420 | 60.495 | 39.791 | 115.334 | 1.00 | 36.85 | A | C |
| ATOM | 1071 | CD2 | TYR A | 420 | 60.464 | 37.674 | 117.128 | 1.00 | 35.51 | A | C |
| ATOM | 1072 | CE2 | TYR A | 420 | 61.215 | 38.804 | 117.409 | 1.00 | 38.35 | A | C |
| ATOM | 1073 | CZ | TYR A | 420 | 61.228 | 39.858 | 116.512 | 1.00 | 40.13 | A | C |
| ATOM | 1074 | OH | TYR A | 420 | 61.973 | 40.976 | 116.801 | 1.00 | 41.02 | A | O |
| ATOM | 1075 | C | TYR A | 420 | 60.679 | 34.746 | 114.812 | 1.00 | 22.16 | A | C |
| ATOM | 1076 | O | TYR A | 420 | 60.583 | 33.744 | 115.515 | 1.00 | 20.07 | A | O |
| ATOM | 1077 | N | PRO A | 421 | 61.863 | 35.172 | 114.347 | 1.00 | 23.33 | A | N |
| ATOM | 1078 | CD | PRO A | 421 | 62.072 | 36.447 | 113.634 | 1.00 | 24.71 | A | C |
| ATOM | 1079 | CA | PRO A | 421 | 63.148 | 34.501 | 114.591 | 1.00 | 24.11 | A | C |
| ATOM | 1080 | CB | PRO A | 421 | 64.129 | 35.342 | 113.773 | 1.00 | 24.83 | A | C |
| ATOM | 1081 | CG | PRO A | 421 | 63.542 | 36.703 | 113.850 | 1.00 | 23.46 | A | C |
| ATOM | 1082 | C | PRO A | 421 | 63.624 | 34.301 | 116.028 | 1.00 | 24.67 | A | C |
| ATOM | 1083 | O | PRO A | 421 | 64.649 | 33.659 | 116.258 | 1.00 | 23.19 | A | O |
| ATOM | 1084 | N | MET A | 422 | 62.870 | 34.827 | 116.986 | 1.00 | 23.77 | A | N |
| ATOM | 1085 | CA | MET A | 422 | 63.228 | 34.732 | 118.393 | 1.00 | 24.04 | A | C |
| ATOM | 1086 | CB | MET A | 422 | 63.351 | 36.148 | 118.961 | 1.00 | 29.95 | A | C |
| ATOM | 1087 | CG | MET A | 422 | 63.671 | 36.231 | 120.433 | 1.00 | 39.49 | A | C |
| ATOM | 1088 | SD | MET A | 422 | 63.479 | 37.927 | 121.034 | 1.00 | 49.06 | A | S |
| ATOM | 1089 | CE | MET A | 422 | 61.893 | 37.842 | 121.866 | 1.00 | 39.08 | A | C |
| ATOM | 1090 | C | MET A | 422 | 62.181 | 33.938 | 119.171 | 1.00 | 22.85 | A | C |
| ATOM | 1091 | O | MET A | 422 | 60.983 | 34.062 | 118.915 | 1.00 | 21.77 | A | O |
| ATOM | 1092 | N | TYR A | 423 | 62.626 | 33.117 | 120.114 | 1.00 | 19.77 | A | N |
| ATOM | 1093 | CA | TYR A | 423 | 61.699 | 32.324 | 120.913 | 1.00 | 21.78 | A | C |
| ATOM | 1094 | CB | TYR A | 423 | 62.441 | 31.214 | 121.659 | 1.00 | 19.09 | A | C |
| ATOM | 1095 | CG | TYR A | 423 | 61.551 | 30.425 | 122.599 | 1.00 | 18.13 | A | C |
| ATOM | 1096 | CD1 | TYR A | 423 | 60.797 | 29.344 | 122.139 | 1.00 | 18.66 | A | C |
| ATOM | 1097 | CE1 | TYR A | 423 | 59.962 | 28.623 | 122.996 | 1.00 | 16.35 | A | C |
| ATOM | 1098 | CD2 | TYR A | 423 | 61.445 | 30.769 | 123.946 | 1.00 | 17.67 | A | C |
| ATOM | 1099 | CE2 | TYR A | 423 | 60.612 | 30.059 | 124.811 | 1.00 | 17.18 | A | C |
| ATOM | 1100 | CZ | TYR A | 423 | 59.874 | 28.987 | 124.330 | 1.00 | 19.92 | A | C |
| ATOM | 1101 | OH | TYR A | 423 | 59.053 | 28.278 | 125.182 | 1.00 | 18.58 | A | O |
| ATOM | 1102 | C | TYR A | 423 | 60.964 | 33.168 | 121.950 | 1.00 | 23.28 | A | C |
| ATOM | 1103 | O | TYR A | 423 | 61.568 | 34.001 | 122.616 | 1.00 | 23.95 | A | O |
| ATOM | 1104 | N | ARG A | 424 | 59.662 | 32.942 | 122.087 | 1.00 | 25.46 | A | N |
| ATOM | 1105 | CA | ARG A | 424 | 58.869 | 33.651 | 123.084 | 1.00 | 31.01 | A | C |
| ATOM | 1106 | CB | ARG A | 424 | 58.376 | 35.005 | 122.550 | 1.00 | 35.79 | A | C |
| ATOM | 1107 | CG | ARG A | 424 | 57.230 | 34.932 | 121.573 | 1.00 | 43.60 | A | C |
| ATOM | 1108 | CD | ARG A | 424 | 56.693 | 36.313 | 121.235 | 1.00 | 50.63 | A | C |
| ATOM | 1109 | NE | ARG A | 424 | 55.481 | 36.228 | 120.420 | 1.00 | 58.11 | A | N |
| ATOM | 1110 | CZ | ARG A | 424 | 54.340 | 35.676 | 120.828 | 1.00 | 62.35 | A | C |
| ATOM | 1111 | NH1 | ARG A | 424 | 54.247 | 35.158 | 122.049 | 1.00 | 65.08 | A | N |
| ATOM | 1112 | NH2 | ARG A | 424 | 53.291 | 35.634 | 120.015 | 1.00 | 62.97 | A | N |
| ATOM | 1113 | C | ARG A | 424 | 57.690 | 32.777 | 123.499 | 1.00 | 29.80 | A | C |
| ATOM | 1114 | O | ARG A | 424 | 56.830 | 32.450 | 122.691 | 1.00 | 29.90 | A | O |
| ATOM | 1115 | N | PHE A | 425 | 57.654 | 32.374 | 124.762 | 1.00 | 29.63 | A | N |
| ATOM | 1116 | CA | PHE A | 425 | 56.555 | 31.534 | 125.203 | 1.00 | 28.26 | A | C |
| ATOM | 1117 | CB | PHE A | 425 | 56.807 | 30.963 | 126.596 | 1.00 | 24.65 | A | C |
| ATOM | 1118 | CG | PHE A | 425 | 55.660 | 30.152 | 127.112 | 1.00 | 23.69 | A | C |
| ATOM | 1119 | CD1 | PHE A | 425 | 54.673 | 30.740 | 127.898 | 1.00 | 22.35 | A | C |
| ATOM | 1120 | CD2 | PHE A | 425 | 55.506 | 28.820 | 126.729 | 1.00 | 21.86 | A | C |
| ATOM | 1121 | CE1 | PHE A | 425 | 53.542 | 30.016 | 128.288 | 1.00 | 23.01 | A | C |
| ATOM | 1122 | CE2 | PHE A | 425 | 54.379 | 28.086 | 127.113 | 1.00 | 21.25 | A | C |
| ATOM | 1123 | CZ | PHE A | 425 | 53.395 | 28.684 | 127.892 | 1.00 | 21.91 | A | C |
| ATOM | 1124 | C | PHE A | 425 | 55.236 | 32.287 | 125.205 | 1.00 | 29.92 | A | C |
| ATOM | 1125 | O | PHE A | 425 | 55.177 | 33.475 | 125.525 | 1.00 | 32.27 | A | O |
| ATOM | 1126 | N | THR A | 426 | 54.172 | 31.586 | 124.843 | 1.00 | 28.15 | A | N |
| ATOM | 1127 | CA | THR A | 426 | 52.858 | 32.195 | 124.817 | 1.00 | 28.89 | A | C |
| ATOM | 1128 | CB | THR A | 426 | 52.581 | 32.896 | 123.463 | 1.00 | 30.65 | A | C |
| ATOM | 1129 | OG1 | THR A | 426 | 51.258 | 33.445 | 123.476 | 1.00 | 30.50 | A | O |
| ATOM | 1130 | CG2 | THR A | 426 | 52.692 | 31.913 | 122.309 | 1.00 | 27.51 | A | C |
| ATOM | 1131 | C | THR A | 426 | 51.771 | 31.168 | 125.054 | 1.00 | 28.39 | A | C |
| ATOM | 1132 | O | THR A | 426 | 51.936 | 29.991 | 124.763 | 1.00 | 27.43 | A | O |
| ATOM | 1133 | N | GLU A | 427 | 50.662 | 31.627 | 125.615 | 1.00 | 31.92 | A | N |
| ATOM | 1134 | CA | GLU A | 427 | 49.515 | 30.767 | 125.864 | 1.00 | 33.81 | A | C |
| ATOM | 1135 | CB | GLU A | 427 | 48.931 | 31.032 | 127.252 | 1.00 | 35.60 | A | C |
| ATOM | 1136 | CO | GLU A | 427 | 49.820 | 30.574 | 128.402 | 1.00 | 41.56 | A | C |
| ATOM | 1137 | CD | GLU A | 427 | 49.178 | 30.786 | 129.765 | 1.00 | 43.70 | A | C |
| ATOM | 1138 | OE1 | GLU A | 427 | 48.985 | 31.959 | 130.162 | 1.00 | 45.79 | A | O |
| ATOM | 1139 | OE2 | GLU A | 427 | 48.865 | 29.779 | 130.437 | 1.00 | 43.08 | A | O |
| ATOM | 1140 | C | GLU A | 427 | 48.501 | 31.133 | 124.785 | 1.00 | 33.55 | A | C |
| ATOM | 1141 | O | GLU A | 427 | 48.634 | 32.165 | 124.127 | 1.00 | 32.48 | A | O |
| ATOM | 1142 | N | GLY A | 428 | 47.492 | 30.297 | 124.596 | 1.00 | 32.38 | A | N |
| ATOM | 1143 | CA | GLY A | 428 | 46.509 | 30.596 | 123.575 | 1.00 | 34.96 | A | C |
| ATOM | 1144 | C | GLY A | 428 | 47.015 | 30.234 | 122.189 | 1.00 | 35.38 | A | C |
| ATOM | 1145 | O | GLY A | 428 | 48.137 | 29.740 | 122.050 | 1.00 | 34.55 | A | O |
| ATOM | 1146 | N | PRO A | 429 | 46.210 | 30.482 | 121.139 | 1.00 | 35.27 | A | N |
| ATOM | 1147 | CD | PRO A | 429 | 44.979 | 31.286 | 121.203 | 1.00 | 35.48 | A | C |
| ATOM | 1148 | CA | PRO A | 429 | 46.555 | 30.185 | 119.744 | 1.00 | 32.90 | A | C |
| ATOM | 1149 | CB | PRO A | 429 | 45.396 | 30.803 | 118.966 | 1.00 | 33.89 | A | C |
| ATOM | 1150 | CG | PRO A | 429 | 44.964 | 31.931 | 119.848 | 1.00 | 34.64 | A | C |
| ATOM | 1151 | C | PRO A | 429 | 47.913 | 30.740 | 119.329 | 1.00 | 30.15 | A | C |
| ATOM | 1152 | O | PRO A | 429 | 48.156 | 31.939 | 119.403 | 1.00 | 32.20 | A | O |
| ATOM | 1153 | N | PRO A | 430 | 48.817 | 29.858 | 118.886 | 1.00 | 27.85 | A | N |
| ATOM | 1154 | CD | PRO A | 430 | 48.637 | 28.397 | 118.903 | 1.00 | 26.06 | A | C |
| ATOM | 1155 | CA | PRO A | 430 | 50.173 | 30.211 | 118.450 | 1.00 | 27.78 | A | C |
| ATOM | 1156 | CB | PRO A | 430 | 50.869 | 28.849 | 118.335 | 1.00 | 28.30 | A | C |
| ATOM | 1157 | CG | PRO A | 430 | 50.029 | 27.927 | 119.193 | 1.00 | 29.20 | A | C |
| ATOM | 1158 | C | PRO A | 430 | 50.260 | 30.989 | 117.135 | 1.00 | 27.63 | A | C |
| ATOM | 1159 | O | PRO A | 430 | 51.068 | 31.909 | 117.000 | 1.00 | 26.32 | A | O |
| ATOM | 1160 | N | LEU A | 431 | 49.437 | 30.611 | 116.164 | 1.00 | 26.85 | A | N |
| ATOM | 1161 | CA | LEU A | 431 | 49.470 | 31.257 | 114.858 | 1.00 | 28.30 | A | C |
| ATOM | 1162 | CB | LEU A | 431 | 48.752 | 30.395 | 113.815 | 1.00 | 26.29 | A | C |
| ATOM | 1163 | CG | LEU A | 431 | 49.353 | 29.024 | 113.507 | 1.00 | 26.78 | A | C |
| ATOM | 1164 | CD1 | LEU A | 431 | 48.615 | 28.430 | 112.326 | 1.00 | 27.79 | A | C |
| ATOM | 1165 | CD2 | LEU A | 431 | 50.827 | 29.144 | 113.188 | 1.00 | 25.85 | A | C |
| ATOM | 1166 | C | LEU A | 431 | 48.897 | 32.658 | 114.815 | 1.00 | 26.95 | A | C |
| ATOM | 1167 | O | LEU A | 431 | 47.776 | 32.891 | 115.248 | 1.00 | 27.17 | A | O |
| ATOM | 1168 | N | HIS A | 432 | 49.681 | 33.585 | 114.279 | 1.00 | 27.31 | A | N |
| ATOM | 1169 | CA | HIS A | 432 | 49.262 | 34.971 | 114.144 | 1.00 | 29.66 | A | C |
| ATOM | 1170 | CB | HIS A | 432 | 50.469 | 35.883 | 114.367 | 1.00 | 30.80 | A | C |
| ATOM | 1171 | CG | HIS A | 432 | 50.157 | 37.342 | 114.263 | 1.00 | 35.25 | A | C |
| ATOM | 1172 | CD2 | HIS A | 432 | 50.006 | 38.291 | 115.216 | 1.00 | 36.05 | A | C |
| ATOM | 1173 | ND1 | HIS A | 432 | 49.973 | 37.980 | 113.056 | 1.00 | 35.61 | A | N |
| ATOM | 1174 | CE1 | HIS A | 432 | 49.724 | 39.260 | 113.269 | 1.00 | 35.01 | A | C |
| ATOM | 1175 | NE2 | HIS A | 432 | 49.739 | 39.474 | 114.571 | 1.00 | 37.63 | A | N |
| ATOM | 1176 | C | HIS A | 432 | 48.688 | 35.148 | 112.729 | 1.00 | 31.08 | A | C |
| ATOM | 1177 | O | HIS A | 432 | 48.989 | 34.353 | 111.843 | 1.00 | 31.29 | A | O |
| ATOM | 1178 | N | LYS A | 433 | 47.855 | 36.169 | 112.525 | 1.00 | 30.77 | A | N |
| ATOM | 1179 | CA | LYS A | 433 | 47.252 | 36.437 | 111.214 | 1.00 | 31.04 | A | C |
| ATOM | 1180 | CB | LYS A | 433 | 46.590 | 37.817 | 111.186 | 1.00 | 36.69 | A | C |
| ATOM | 1181 | CG | LYS A | 433 | 45.486 | 38.010 | 112.205 | 1.00 | 46.32 | A | C |
| ATOM | 1182 | CD | LYS A | 433 | 45.180 | 39.489 | 112.428 | 1.00 | 52.39 | A | C |
| ATOM | 1183 | CE | LYS A | 433 | 44.194 | 39.680 | 113.585 | 1.00 | 57.26 | A | C |
| ATOM | 1184 | NZ | LYS A | 433 | 43.913 | 41.116 | 113.889 | 1.00 | 59.05 | A | N |
| ATOM | 1185 | C | LYS A | 433 | 48.328 | 36.411 | 110.148 | 1.00 | 30.86 | A | C |
| ATOM | 1186 | O | LYS A | 433 | 48.111 | 35.954 | 109.025 | 1.00 | 28.27 | A | O |
| ATOM | 1187 | N | ASP A | 434 | 49.496 | 36.919 | 110.511 | 1.00 | 29.06 | A | N |
| ATOM | 1188 | CA | ASP A | 434 | 50.609 | 36.960 | 109.591 | 1.00 | 26.69 | A | C |
| ATOM | 1189 | CB | ASP A | 434 | 51.766 | 37.732 | 110.213 | 1.00 | 26.22 | A | C |
| ATOM | 1190 | CG | ASP A | 434 | 52.923 | 37.905 | 109.258 | 1.00 | 27.32 | A | C |
| ATOM | 1191 | OD1 | ASP A | 434 | 53.916 | 37.168 | 109.388 | 1.00 | 27.96 | A | O |
| ATOM | 1192 | OD2 | ASP A | 434 | 52.837 | 38.774 | 108.366 | 1.00 | 29.60 | A | O |
| ATOM | 1193 | C | ASP A | 434 | 51.052 | 35.557 | 109.202 | 1.00 | 27.04 | A | C |
| ATOM | 1194 | O | ASP A | 434 | 51.350 | 35.300 | 108.039 | 1.00 | 26.95 | A | O |
| ATOM | 1195 | N | ASP A | 435 | 51.086 | 34.644 | 110.169 | 1.00 | 26.04 | A | N |
| ATOM | 1196 | CA | ASP A | 435 | 51.496 | 33.270 | 109.890 | 1.00 | 26.18 | A | C. |
| ATOM | 1197 | CB | ASP A | 435 | 51.629 | 32.453 | 111.186 | 1.00 | 25.76 | A | C |
| ATOM | 1198 | CG | ASP A | 435 | 52.659 | 33.026 | 112.159 | 1.00 | 23.37 | A | C |
| ATOM | 1199 | OD1 | ASP A | 435 | 53.833 | 33.231 | 111.778 | 1.00 | 17.56 | A | O |
| ATOM | 1200 | OD2 | ASP A | 435 | 52.281 | 33.257 | 113.319 | 1.00 | 21.37 | A | O |
| ATOM | 1201 | C | ASP A | 435 | 50.483 | 32.586 | 108.979 | 1.00 | 26.53 | A | C |
| ATOM | 1202 | O | ASP A | 435 | 50.857 | 31.854 | 108.065 | 1.00 | 27.59 | A | O |
| ATOM | 1203 | N | VAL A | 436 | 49.202 | 32.824 | 109.243 | 1.00 | 26.10 | A | N |
| ATOM | 1204 | CA | VAL A | 436 | 48.123 | 32.238 | 108.461 | 1.00 | 27.17 | A | C |
| ATOM | 1205 | CB | VAL A | 436 | 46.758 | 32.516 | 109.116 | 1.00 | 28.67 | A | C |
| ATOM | 1206 | CG1 | VAL A | 436 | 45.651 | 31.841 | 108.322 | 1.00 | 28.01 | A | C |
| ATOM | 1207 | CG2 | VAL A | 436 | 46.760 | 32.012 | 110.546 | 1.00 | 27.09 | A | C |
| ATOM | 1208 | C | VAL A | 436 | 48.105 | 32.755 | 107.021 | 1.00 | 28.36 | A | C |
| ATOM | 1209 | O | VAL A | 436 | 47.814 | 32.006 | 106.096 | 1.00 | 28.48 | A | O |
| ATOM | 1210 | N | ASN A | 437 | 48.411 | 34.034 | 106.832 | 1.00 | 30.05 | A | N |
| ATOM | 1211 | CA | ASN A | 437 | 48.449 | 34.604 | 105.488 | 1.00 | 32.08 | A | C |
| ATOM | 1212 | CB | ASN A | 437 | 48.670 | 36.121 | 105.525 | 1.00 | 36.19 | A | C |
| ATOM | 1213 | CG | ASN A | 437 | 47.464 | 36.875 | 106.018 | 1.00 | 40.19 | A | C |
| ATOM | 1214 | OD1 | ASN A | 437 | 46.328 | 36.484 | 105.759 | 1.00 | 44.41 | A | O |
| ATOM | 1215 | ND2 | ASN A | 437 | 47.701 | 37.978 | 106.719 | 1.00 | 44.69 | A | N |
| ATOM | 1216 | C | ASN A | 437 | 49.589 | 33.983 | 104.701 | 1.00 | 30.92 | A | C |
| ATOM | 1217 | O | ASN A | 437 | 49.406 | 33.559 | 103.565 | 1.00 | 31.67 | A | O |
| ATOM | 1218 | N | GLY A | 438 | 50.768 | 33.946 | 105.315 | 1.00 | 27.54 | A | N |
| ATOM | 1219 | CA | GLY A | 438 | 51.931 | 33.385 | 104.659 | 1.00 | 25.40 | A | C |
| ATOM | 1220 | C | GLY A | 438 | 51.755 | 31.952 | 104.205 | 1.00 | 24.52 | A | C |
| ATOM | 1221 | O | GLY A | 438 | 52.171 | 31.593 | 103.110 | 1.00 | 26.30 | A | O |
| ATOM | 1222 | N | ILE A | 439 | 51.137 | 31.125 | 105.039 | 1.00 | 23.54 | A | N |
| ATOM | 1223 | CA | ILE A | 439 | 50.943 | 29.732 | 104.681 | 1.00 | 23.31 | A | C |
| ATOM | 1224 | CB | ILE A | 439 | 50.619 | 28.871 | 105.944 | 1.00 | 20.22 | A | C |
| ATOM | 1225 | CG2 | ILE A | 439 | 49.210 | 29.136 | 106.450 | 1.00 | 18.09 | A | C |
| ATOM | 1226 | CG1 | ILE A | 439 | 50.814 | 27.393 | 105.614 | 1.00 | 21.12 | A | C |
| ATOM | 1227 | CD1 | ILE A | 439 | 52.238 | 27.048 | 105.201 | 1.00 | 14.56 | A | C |
| ATOM | 1228 | C | ILE A | 439 | 49.867 | 29.547 | 103.593 | 1.00 | 24.64 | A | C |
| ATOM | 1229 | O | ILE A | 439 | 50.017 | 28.712 | 102.705 | 1.00 | 25.35 | A | O |
| ATOM | 1230 | N | ARG A | 440 | 48.793 | 30.324 | 103.649 | 1.00 | 23.43 | A | N |
| ATOM | 1231 | CA | ARG A | 440 | 47.751 | 30.217 | 102.638 | 1.00 | 24.74 | A | C |
| ATOM | 1232 | CB | ARG A | 440 | 46.481 | 30.932 | 103.091 | 1.00 | 25.89 | A | C |
| ATOM | 1233 | CG | ARG A | 440 | 45.673 | 30.154 | 104.093 | 1.00 | 28.05 | A | C |
| ATOM | 1234 | CD | ARG A | 440 | 44.453 | 30.927 | 104.518 | 1.00 | 33.06 | A | C |
| ATOM | 1235 | NE | ARG A | 440 | 43.678 | 30.183 | 105.502 | 1.00 | 40.49 | A | N |
| ATOM | 1236 | CZ | ARG A | 440 | 42.752 | 30.722 | 106.286 | 1.00 | 43.77 | A | C |
| ATOM | 1237 | NH1 | ARG A | 440 | 42.485 | 32.020 | 106.205 | 1.00 | 45.47 | A | N |
| ATOM | 1238 | NH2 | ARG A | 440 | 42.089 | 29.964 | 107.150 | 1.00 | 43.22 | A | N |
| ATOM | 1239 | C | ARO A | 440 | 48.217 | 30.806 | 101.312 | 1.00 | 26.05 | A | C |
| ATOM | 1240 | O | ARG A | 440 | 47.691 | 30.466 | 100.257 | 1.00 | 29.07 | A | O |
| ATOM | 1241 | N | HIS A | 441 | 49.201 | 31.694 | 101.360 | 1.00 | 24.35 | A | N |
| ATOM | 1242 | CA | HIS A | 441 | 49.707 | 32.290 | 100.139 | 1.00 | 24.72 | A | C |
| ATOM | 1243 | CB | HIS A | 441 | 50.628 | 33.465 | 100.450 | 1.00 | 21.94 | A | C |
| ATOM | 1244 | CG | HIS A | 441 | 50.942 | 34.308 | 99.255 | 1.00 | 21.49 | A | C |
| ATOM | 1245 | CD2 | HIS A | 441 | 52.068 | 34.421 | 98.513 | 1.00 | 23.26 | A | C |
| ATOM | 1246 | ND1 | HIS A | 441 | 50.018 | 35.152 | 98.679 | 1.00 | 23.21 | A | N |
| ATOM | 1247 | CE1 | HIS A | 441 | 50.561 | 35.748 | 97.634 | 1.00 | 21.93 | A | C |
| ATOM | 1248 | NE2 | HIS A | 441 | 51.804 | 35.322 | 97.512 | 1.00 | 24.09 | A | N |
| ATOM | 1249 | C | HIS A | 441 | 50.463 | 31.238 | 99.332 | 1.00 | 26.24 | A | C |
| ATOM | 1250 | O | HIS A | 441 | 50.531 | 31.313 | 98.104 | 1.00 | 26.96 | A | O |
| ATOM | 1251 | N | LEU A | 442 | 51.028 | 30.260 | 100.032 | 1.00 | 27.09 | A | N |
| ATOM | 1252 | CA | LEU A | 442 | 51.770 | 29.178 | 99.394 | 1.00 | 27.80 | A | C |
| ATOM | 1253 | CB | LEU A | 442 | 52.861 | 28.648 | 100.329 | 1.00 | 28.49 | A | C |
| ATOM | 1254 | CG | LEU A | 442 | 54.042 | 29.552 | 100.679 | 1.00 | 30.92 | A | C |
| ATOM | 1255 | CD1 | LEU A | 442 | 54.898 | 28.875 | 101.737 | 1.00 | 31.60 | A | C |
| ATOM | 1256 | CD2 | LEU A | 442 | 54.863 | 29.836 | 99.434 | 1.00 | 31.89 | A | C |
| ATOM | 1257 | C | LEU A | 442 | 50.877 | 28.009 | 98.984 | 1.00 | 28.59 | A | C |
| ATOM | 1258 | O | LEU A | 442 | 51.084 | 27.420 | 97.929 | 1.00 | 28.30 | A | O |
| ATOM | 1259 | N | TYR A | 443 | 49.896 | 27.674 | 99.821 | 1.00 | 30.49 | A | N |
| ATOM | 1260 | CA | TYR A | 443 | 48.993 | 26.553 | 99.548 | 1.00 | 31.37 | A | C |
| ATOM | 1261 | CB | TYR A | 443 | 49.181 | 25.450 | 100.601 | 1.00 | 30.85 | A | C |
| ATOM | 1262 | CG | TYR A | 443 | 50.611 | 24.990 | 100.753 | 1.00 | 28.19 | A | C |
| ATOM | 1263 | CD1 | TYR A | 443 | 51.462 | 25.575 | 101.697 | 1.00 | 25.52 | A | C |
| ATOM | 1264 | CE1 | TYR A | 443 | 52.805 | 25.190 | 101.793 | 1.00 | 23.19 | A | C |
| ATOM | 1265 | CD2 | TYR A | 443 | 51.133 | 24.007 | 99.913 | 1.00 | 27.48 | A | C |
| ATOM | 1266 | CE2 | TYR A | 443 | 52.469 | 23.617 | 100.000 | 1.00 | 26.82 | A | C |
| ATOM | 1267 | CZ | TYR A | 443 | 53.298 | 24.213 | 100.937 | 1.00 | 24.13 | A | C |
| ATOM | 1268 | OH | TYR A | 443 | 54.618 | 23.837 | 100.986 | 1.00 | 24.42 | A | O |
| ATOM | 1269 | C | TYR A | 443 | 47.518 | 26.945 | 99.497 | 1.00 | 31.76 | A | C |
| ATOM | 1270 | O | TYR A | 443 | 46.719 | 26.112 | 99.036 | 1.00 | 33.61 | A | O |
| ATOM | 1271 | OT | TYR A | 443 | 47.163 | 28.054 | 99.935 | 1.00 | 33.91 | A | O |
| ATOM | 2560 | ZN | ZN A | 1 | 63.267 | 29.950 | 112.382 | 1.00 | 18.93 | Z | ZN |
| ATOM | 2561 | ZN | ZN A | 2 | 70.729 | 19.617 | 111.673 | 1.00 | 22.07 | Z | ZN |
| ATOM | 2565 | CA | CA A | 3 | 71.413 | 25.140 | 121.665 | 1.00 | 25.39 | C | C |
| ATOM | 2566 | CA | CA A | 4 | 63.107 | 15.170 | 103.757 | 1.00 | 28.86 | C | C |
| ATOM | 2567 | CA | CA A | 5 | 67.687 | 17.476 | 126.440 | 1.00 | 49.10 | C | C |
| ATOM | 2568 | CA | CA A | 6 | 57.389 | 24.336 | 131.008 | 1.00 | 56.13 | C | C |
| ATOM | 2573 | CA | CA A | 7 | 41.603 | 20.860 | 107.667 | 1.00 | 99.94 | C | C |
| ATOM | 2515 | C1 | FRA A | 1 | 62.295 | 30.885 | 117.488 | 1.00 | 16.45 | M | C |
| ATOM | 2516 | C2 | FRA A | 1 | 63.038 | 29.608 | 117.942 | 1.00 | 19.42 | M | C |
| ATOM | 2517 | C3 | FRA A | 1 | 61.951 | 28.564 | 118.122 | 1.00 | 18.32 | M | C |
| ATOM | 2518 | C4 | FRA A | 1 | 64.019 | 29.134 | 116.841 | 1.00 | 18.44 | M | C |
| ATOM | 2519 | C5 | FRA A | 1 | 65.268 | 30.026 | 116.452 | 1.00 | 20.30 | M | C |
| ATOM | 2520 | C6 | FRA A | 1 | 66.093 | 29.344 | 115.298 | 1.00 | 21.58 | M | C |
| ATOM | 2521 | N7 | FRA A | 1 | 65.323 | 29.140 | 114.074 | 1.00 | 19.54 | M | N |
| ATOM | 2522 | O8 | FRA A | 1 | 65.083 | 30.177 | 113.358 | 1.00 | 20.40 | M | O |
| ATOM | 2523 | C9 | FRA A | 1 | 64.851 | 27.958 | 113.673 | 1.00 | 20.49 | M | C |
| ATOM | 2524 | O10 | FRA A | 1 | 64.213 | 27.793 | 112.689 | 1.00 | 19.70 | M | O |
| ATOM | 2525 | C11 | FRA A | 1 | 66.274 | 30.187 | 117.629 | 1.00 | 22.72 | M | C |
| ATOM | 2526 | N12 | FRA A | 1 | 66.450 | 31.428 | 118.131 | 1.00 | 21.65 | M | N |
| ATOM | 2527 | C13 | FRA A | 1 | 67.389 | 31.754 | 119.271 | 1.00 | 23.60 | M | C |
| ATOM | 2528 | C14 | FRA A | 1 | 68.279 | 33.067 | 118.969 | 1.00 | 24.52 | M | C |
| ATOM | 2529 | C15 | FRA A | 1 | 69.263 | 33.407 | 120.153 | 1.00 | 23.38 | M | C |
| ATOM | 2530 | C16 | FRA A | 1 | 67.424 | 34.373 | 118.736 | 1.00 | 23.95 | M | C |
| ATOM | 2531 | C17 | FRA A | 1 | 69.119 | 32.883 | 117.677 | 1.00 | 21.36 | M | C |
| ATOM | 2532 | C18 | FRA A | 1 | 66.491 | 31.847 | 120.586 | 1.00 | 23.15 | M | C |
| ATOM | 2533 | O19 | FRA A | 1 | 65.406 | 32.468 | 120.589 | 1.00 | 24.19 | M | O |
| ATOM | 2534 | N20 | FRA A | 1 | 67.003 | 31.196 | 121.667 | 1.00 | 21.19 | M | N |
| ATOM | 2535 | C21 | FRA A | 1 | 66.350 | 31.137 | 122.996 | 1.00 | 25.36 | M | C |
| ATOM | 2536 | O22 | FRA A | 1 | 66.882 | 29.180 | 118.067 | 1.00 | 22.29 | M | O |
| ATOM | 1273 | CB | ASP B | 110 | 39.115 | 26.293 | 159.737 | 1.00 | 61.05 | B | C |
| ATOM | 1274 | CG | ASP B | 110 | 39.042 | 27.399 | 160.776 | 1.00 | 62.86 | B | C |
| ATOM | 1275 | OD1 | ASP B | 110 | 40.101 | 27.786 | 161.322 | 1.00 | 62.33 | B | O |
| ATOM | 1276 | OD2 | ASP B | 110 | 37.917 | 27.876 | 161.046 | 1.00 | 63.37 | B | O |
| ATOM | 1277 | C | ASP B | 110 | 40.865 | 24.867 | 160.849 | 1.00 | 56.49 | B | C |
| ATOM | 1278 | O | ASP B | 110 | 41.355 | 25.782 | 161.519 | 1.00 | 56.57 | B | O |
| ATOM | 1279 | N | ASP B | 110 | 38.439 | 24.617 | 161.434 | 1.00 | 58.69 | B | N |
| ATOM | 1280 | CA | ASP B | 110 | 39.418 | 24.920 | 160.351 | 1.00 | 58.71 | B | C |
| ATOM | 1281 | N | LEU B | 111 | 41.541 | 23.778 | 160.512 | 1.00 | 52.64 | B | N |
| ATOM | 1282 | CA | LEU B | 111 | 42.921 | 23.579 | 160.910 | 1.00 | 48.28 | B | C |
| ATOM | 1283 | CB | LEU B | 111 | 43.265 | 22.094 | 160.794 | 1.00 | 48.88 | B | C |
| ATOM | 1284 | CG | LEU B | 111 | 44.509 | 21.605 | 161.528 | 1.00 | 50.33 | B | C |
| ATOM | 1285 | CD1 | LEU B | 111 | 44.535 | 22.156 | 162.948 | 1.00 | 50.73 | B | C |
| ATOM | 1286 | CD2 | LEU B | 111 | 44.500 | 20.088 | 161.540 | 1.00 | 51.89 | B | C |
| ATOM | 1287 | C | LEU B | 111 | 43.846 | 24.419 | 160.029 | 1.00 | 43.58 | B | C |
| ATOM | 1288 | O | LEU B | 111 | 43.584 | 24.607 | 158.838 | 1.00 | 42.86 | B | O |
| ATOM | 1289 | N | LYS B | 112 | 44.917 | 24.942 | 160.615 | 1.00 | 37.78 | B | N |
| ATOM | 1290 | CA | LYS B | 112 | 45.858 | 25.745 | 159.846 | 1.00 | 34.84 | B | C |
| ATOM | 1291 | CB | LYS B | 112 | 45.341 | 27.176 | 159.702 | 1.00 | 35.26 | B | C |
| ATOM | 1292 | CG | LYS B | 112 | 45.439 | 28.007 | 160.972 | 1.00 | 39.03 | B | C |
| ATOM | 1293 | CD | LYS B | 112 | 45.160 | 29.466 | 160.674 | 1.00 | 38.56 | B | C |
| ATOM | 1294 | CE | LYS B | 112 | 45.529 | 30.353 | 161.845 | 1.00 | 41.04 | B | C |
| ATOM | 1295 | NZ | LYS B | 112 | 45.396 | 31.790 | 161.475 | 1.00 | 41.35 | B | N |
| ATOM | 1296 | C | LYS B | 112 | 47.233 | 25.767 | 160.498 | 1.00 | 32.44 | B | C |
| ATOM | 1297 | O | LYS B | 112 | 47.395 | 25.317 | 161.628 | 1.00 | 31.94 | B | O |
| ATOM | 1298 | N | TRP B | 113 | 48.225 | 26.284 | 159.779 | 1.00 | 30.35 | B | N |
| ATOM | 1299 | CA | TRP B | 113 | 49.571 | 26.359 | 160.320 | 1.00 | 30.31 | B | C |
| ATOM | 1300 | CB | TRP B | 113 | 50.613 | 26.516 | 159.204 | 1.00 | 27.93 | B | C |
| ATOM | 1301 | CG | TRP B | 113 | 50.690 | 25.351 | 158.258 | 1.00 | 27.86 | B | C |
| ATOM | 1302 | CD2 | TRP B | 113 | 51.204 | 24.040 | 158.541 | 1.00 | 26.96 | B | C |
| ATOM | 1303 | CE2 | TRP B | 113 | 51.058 | 23.271 | 157.363 | 1.00 | 27.Z9 | B | C |
| ATOM | 1304 | CE3 | TRP B | 113 | 51.773 | 23.441 | 159.674 | 1.00 | 23.04 | B | C |
| ATOM | 1305 | CD1 | TRP B | 113 | 50.266 | 25.321 | 156.958 | 1.00 | 26.75 | B | C |
| ATOM | 1306 | NE1 | TRP B | 113 | 50.483 | 24.078 | 156.415 | 1.00 | 23.61 | B | N |
| ATOM | 1307 | CZ2 | TRP B | 113 | 51.460 | 21.930 | 157.287 | 1.00 | 24.46 | B | C |
| ATOM | 1308 | CZ3 | TRP B | 113 | 52.171 | 22.108 | 159.597 | 1.00 | 23.32 | B | C |
| ATOM | 1309 | CH2 | TRP B | 113 | 52.012 | 21.369 | 158.410 | 1.00 | 23.78 | B | C |
| ATOM | 1310 | C | TRP B | 113 | 49.645 | 27.558 | 161.252 | 1.00 | 31.82 | B | C |
| ATOM | 1311 | O | TRP B | 113 | 49.024 | 28.589 | 160.996 | 1.00 | 32.51 | B | O |
| ATOM | 1312 | N | HIS B | 114 | 50.400 | 27.419 | 162.335 | 1.00 | 31.20 | B | N |
| ATOM | 1313 | CA | HIS B | 114 | 50.549 | 28.507 | 163.285 | 1.00 | 31.82 | B | C |
| ATOM | 1314 | CB | HIS B | 114 | 50.577 | 27.963 | 164.711 | 1.00 | 31.55 | B | C |
| ATOM | 1315 | CG | HIS B | 114 | 49.237 | 27.513 | 165.193 | 1.00 | 32.54 | B | C |
| ATOM | 1316 | CD2 | HIS B | 114 | 47.999 | 27.711 | 164.681 | 1.00 | 32.52 | B | C |
| ATOM | 1317 | ND1 | HIS B | 114 | 49.064 | 26.767 | 166.337 | 1.00 | 33.18 | B | N |
| ATOM | 1318 | CE1 | HIS B | 114 | 47.777 | 26.523 | 166.510 | 1.00 | 32.64 | B | C |
| ATOM | 1319 | NE2 | HIS B | 114 | 47.110 | 27.085 | 165.518 | 1.00 | 34.29 | B | N |
| ATOM | 1320 | C | HIS B | 114 | 51.774 | 29.357 | 163.006 | 1.00 | 30.66 | B | C |
| ATOM | 1321 | O | HIS B | 114 | 51.997 | 30.358 | 163.679 | 1.00 | 34.99 | B | O |
| ATOM | 1322 | N | HIS B | 115 | 52.572 | 28.950 | 162.022 | 1.00 | 28.55 | B | N |
| ATOM | 1323 | CA | HIS B | 115 | 53.745 | 29.722 | 161.617 | 1.00 | 25.56 | B | C |
| ATOM | 1324 | CB | HIS B | 115 | 55.044 | 28.934 | 161.824 | 1.00 | 25.76 | B | C |
| ATOM | 1325 | CG | HIS B | 115 | 55.057 | 27.591 | 161.164 | 1.00 | 25.72 | B | C |
| ATOM | 1326 | CD2 | HIS B | 115 | 55.839 | 27.090 | 160.180 | 1.00 | 24.52 | B | C |
| ATOM | 1327 | ND1 | HIS B | 115 | 54.198 | 26.576 | 161.526 | 1.00 | 24.79 | B | N |
| ATOM | 1328 | CE1 | HIS B | 115 | 54.453 | 25.506 | 160.796 | 1.00 | 22.93 | B | C |
| ATOM | 1329 | NE2 | HIS B | 115 | 55.445 | 25.791 | 159.971 | 1.00 | 23.07 | B | N |
| ATOM | 1330 | C | HIS B | 115 | 53.553 | 30.058 | 160.144 | 1.00 | 25.78 | B | C |
| ATOM | 1331 | O | HIS B | 115 | 52.775 | 29.406 | 159.456 | 1.00 | 26.11 | B | O |
| ATOM | 1332 | N | HIS B | 116 | 54.261 | 31.072 | 159.666 | 1.00 | 25.82 | B | N |
| ATOM | 1333 | CA | HIS B | 116 | 54.133 | 31.514 | 158.286 | 1.00 | 25.15 | B | C |
| ATOM | 1334 | CB | HIS B | 116 | 53.911 | 33.030 | 158.253 | 1.00 | 25.12 | B | C |
| ATOM | 1335 | CG | HIS B | 116 | 52.559 | 33.446 | 158.740 | 1.00 | 26.86 | B | C |
| ATOM | 1336 | CD2 | HIS B | 116 | 51.495 | 32.712 | 159.144 | 1.00 | 25.46 | B | C |
| ATOM | 1337 | ND1 | HIS B | 116 | 52.177 | 34.766 | 158.840 | 1.00 | 26.98 | B | N |
| ATOM | 1338 | CE1 | HIS B | 116 | 50.934 | 34.826 | 159.285 | 1.00 | 29.50 | B | C |
| ATOM | 1339 | NE2 | HIS B | 116 | 50.498 | 33.594 | 159.477 | 1.00 | 28.22 | B | N |
| ATOM | 1340 | C | HIS B | 116 | 55.290 | 31.152 | 157.372 | 1.00 | 24.84 | B | C |
| ATOM | 1341 | O | HIS B | 116 | 55.163 | 31.228 | 156.155 | 1.00 | 24.51 | B | O |
| ATOM | 1342 | N | ASN B | 117 | 56.421 | 30.779 | 157.955 | 1.00 | 23.92 | B | N |
| ATOM | 1343 | CA | ASN B | 117 | 57.581 | 30.395 | 157.166 | 1.00 | 23.42 | B | C |
| ATOM | 1344 | CB | ASN B | 117 | 58.868 | 30.914 | 157.824 | 1.00 | 24.61 | B | C |
| ATOM | 1345 | CG | ASN B | 117 | 59.019 | 30.486 | 159.285 | 1.00 | 27.08 | B | C |
| ATOM | 1346 | OD1 | ASN B | 117 | 58.047 | 30.395 | 160.041 | 1.00 | 28.75 | B | O |
| ATOM | 1347 | ND2 | ASN B | 117 | 60.259 | 30.244 | 159.689 | 1.00 | 24.54 | B | N |
| ATOM | 1348 | C | ASN B | 117 | 57.568 | 28.878 | 157.046 | 1.00 | 23.79 | B | C |
| ATOM | 1349 | O | ASN B | 117 | 58.249 | 28.160 | 157.779 | 1.00 | 22.99 | B | O |
| ATOM | 1350 | N | ILE B | 118 | 56.760 | 28.401 | 156.107 | 1.00 | 23.12 | B | N |
| ATOM | 1351 | CA | ILE B | 118 | 56.585 | 26.9791 | 55.876 | 1.00 | 24.05 | B | C |
| ATOM | 1352 | CB | ILE B | 118 | 55.245 | 26.7341 | 55.156 | 1.00 | 25.87 | B | C |
| ATOM | 1353 | CG2 | ILE B | 118 | 55.066 | 25.2561 | 54.855 | 1.00 | 25.54 | B | C |
| ATOM | 1354 | CG1 | ILE B | 118 | 54.104 | 27.259 | 156.039 | 1.00 | 26.83 | B | C |
| ATOM | 1355 | CD1 | ILE B | 118 | 52.721 | 27.053 | 155.465 | 1.00 | 31.75 | B | C |
| ATOM | 1356 | C | ILE B | 118 | 57.722 | 26.340 | 155.097 | 1.00 | 23.08 | B | C |
| ATOM | 1357 | O | ILE B | 118 | 58.319 | 26.962 | 154.223 | 1.00 | 25.00 | B | O |
| ATOM | 1358 | N | THR B | 119 | 58.029 | 25.094 | 155.429 | 1.00 | 21.12 | B | N |
| ATOM | 1359 | CA | THR B | 119 | 59.096 | 24.382 | 154.744 | 1.00 | 19.30 | B | C |
| ATOM | 1360 | CB | THR B | 119 | 60.203 | 23.938 | 155.721 | 1.00 | 18.41 | B | C |
| ATOM | 1361 | OG1 | THR B | 119 | 59.640 | 23.097 | 156.732 | 1.00 | 18.81 | B | O |
| ATOM | 1362 | CG2 | THR B | 119 | 60.861 | 25.147 | 156.359 | 1.00 | 14.91 | B | C |
| ATOM | 1363 | C | THR B | 119 | 58.564 | 23.156 | 154.026 | 1.00 | 18.87 | B | C |
| ATOM | 1364 | O | THR B | 119 | 57.539 | 22.586 | 154.415 | 1.00 | 17.08 | B | O |
| ATOM | 1365 | N | TYR B | 120 | 59.260 | 22.756 | 152.966 | 1.00 | 18.82 | B | N |
| ATOM | 1366 | CA | TYR B | 120 | 58.848 | 21.584 | 152.207 | 1.00 | 17.71 | B | C |
| ATOM | 1367 | CB | TYR B | 120 | 57.966 | 21.973 | 151.004 | 1.00 | 14.12 | B | C |
| ATOM | 1368 | CG | TYR B | 120 | 58.671 | 22.725 | 149.891 | 1.00 | 14.85 | B | C |
| ATOM | 1369 | CD1 | TYR B | 120 | 58.811 | 24.113 | 149.936 | 1.00 | 15.43 | B | C |
| ATOM | 1370 | CE1 | TYR B | 120 | 59.392 | 24.817 | 148.882 | 1.00 | 12.87 | B | C |
| ATOM | 1371 | CD2 | TYR B | 120 | 59.144 | 22.052 | 148.761 | 1.00 | 15.15 | B | C |
| ATOM | 1372 | CE2 | TYR B | 120 | 59.725 | 22.746 | 147.702 | 1.00 | 15.56 | B | C |
| ATOM | 1373 | CZ | TYR B | 120 | 59.842 | 24.126 | 147.766 | 1.00 | 17.67 | B | C |
| ATOM | 1374 | OH | TYR B | 120 | 60.378 | 24.816 | 146.702 | 1.00 | 20.47 | B | O |
| ATOM | 1375 | C | TYR B | 120 | 60.048 | 20.798 | 151.726 | 1.00 | 14.81 | B | C |
| ATOM | 1376 | O | TYR B | 120 | 61.116 | 21.353 | 151.497 | 1.00 | 14.65 | B | O |
| ATOM | 1377 | N | TRP B | 121 | 59.840 | 19.499 | 151.567 | 1.00 | 12.91 | B | N |
| ATOM | 1378 | CA | TRP B | 121 | 60.867 | 18.587 | 151.121 | 1.00 | 14.27 | B | C |
| ATOM | 1379 | CB | TRP B | 121 | 61.271 | 17.677 | 152.295 | 1.00 | 16.23 | B | C |
| ATOM | 1380 | CG | TRP B | 121 | 62.237 | 16.559 | 151.970 | 1.00 | 16.27 | B | C |
| ATOM | 1381 | CD2 | TRP B | 121 | 62.292 | 15.266 | 152.595 | 1.00 | 17.47 | B | C |
| ATOM | 1382 | GE2 | TRP B | 121 | 63.424 | 14.600 | 152.078 | 1.00 | 17.45 | B | C |
| ATOM | 1383 | CE3 | TRP B | 121 | 61.496 | 14.610 | 153.548 | 1.00 | 17.63 | B | C |
| ATOM | 1384 | CD1 | TRP B | 121 | 63.303 | 16.613 | 151.117 | 1.00 | 17.04 | B | C |
| ATOM | 1385 | NE1 | TRP B | 121 | 64.021 | 15.442 | 151.179 | 1.00 | 16.74 | B | N |
| ATOM | 1386 | CZ2 | TRP B | 121 | 63.783 | 13.307 | 152.484 | 1.00 | 18.60 | B | C |
| ATOM | 1387 | CZ3 | TRP B | 121 | 61.853 | 13.327 | 153.952 | 1.00 | 18.18 | B | C |
| ATOM | 1388 | CH2 | TRP B | 121 | 62.988 | 12.691 | 153.420 | 1.00 | 18.22 | B | C |
| ATOM | 1389 | C | TRP B | 121 | 60.355 | 17.741 | 149.955 | 1.00 | 15.49 | B | C |
| ATOM | 1390 | O | TRP B | 121 | 59.312 | 17.093 | 150.061 | 1.00 | 13.62 | B | O |
| ATOM | 1391 | N | ILE B | 122 | 61.086 | 17.770 | 148.841 | 1.00 | 12.10 | B | N |
| ATOM | 1392 | CA | ILE B | 122 | 60.729 | 16.956 | 147.696 | 1.00 | 13.59 | B | C |
| ATOM | 1393 | CB | ILE B | 122 | 61.167 | 17.608 | 146.381 | 1.00 | 14.78 | B | C |
| ATOM | 1394 | CG2 | ILE B | 122 | 60.773 | 16.715 | 145.214 | 1.00 | 10.29 | B | C |
| ATOM | 1395 | CG1 | ILE B | 122 | 60.520 | 18.991 | 146.254 | 1.00 | 13.64 | B | C |
| ATOM | 1396 | CD1 | ILE B | 122 | 61.024 | 19.811 | 145.080 | 1.00 | 12.29 | B | C |
| ATOM | 1397 | C | ILE B | 122 | 61.514 | 15.672 | 147.918 | 1.00 | 13.64 | B | C |
| ATOM | 1398 | O | ILE B | 122 | 62.694 | 15.588 | 147.592 | 1.00 | 14.54 | B | O |
| ATOM | 1399 | N | GLN B | 123 | 60.855 | 14.679 | 148.496 | 1.00 | 13.68 | B | N |
| ATOM | 1400 | CA | GLN B | 123 | 61.496 | 13.413 | 148.802 | 1.00 | 15.77 | B | C |
| ATOM | 1401 | CB | GLN B | 123 | 60.564 | 12.573 | 149.662 | 1.00 | 14.28 | B | C |
| ATOM | 1402 | CG | GLN B | 123 | 61.092 | 11.200 | 149.957 | 1.00 | 19.68 | B | C |
| ATOM | 1403 | CD | GLN B | 123 | 60.133 | 10.402 | 150.794 | 1.00 | 22.52 | B | C |
| ATOM | 1404 | OE1 | GLN B | 123 | 59.933 | 9.211 | 150.560 | 1.00 | 28.95 | B | O |
| ATOM | 1405 | NE2 | GLN B | 123 | 59.534 | 11.048 | 151.782 | 1.00 | 19.79 | B | N |
| ATOM | 1406 | C | GLN B | 123 | 61.971 | 12.587 | 147.604 | 1.00 | 18.55 | B | C |
| ATOM | 1407 | O | GLN B | 123 | 63.086 | 12.069 | 147.611 | 1.00 | 18.76 | B | O |
| ATOM | 1408 | N | ASN B | 124 | 61.118 | 12.428 | 146.596 | 1.00 | 19.57 | B | N |
| ATOM | 1409 | CA | ASN B | 124 | 61.492 | 11.664 | 145.412 | 1.00 | 17.91 | B | C |
| ATOM | 1410 | CB | ASN B | 124 | 61.111 | 10.184 | 145.549 | 1.00 | 17.29 | B | C |
| ATOM | 1411 | CG | ASN B | 124 | 59.628 | 9.965 | 145.734 | 1.00 | 18.23 | B | C |
| ATOM | 1412 | OD1 | ASN B | 124 | 58.830 | 10.882 | 145.602 | 1.00 | 20.83 | B | O |
| ATOM | 1413 | ND2 | ASN B | 124 | 59.254 | 8.735 | 146.035 | 1.00 | 18.51 | B | N |
| ATOM | 1414 | C | ASN B | 124 | 60.868 | 12.265 | 144.165 | 1.00 | 19.24 | B | C |
| ATOM | 1415 | O | ASN B | 124 | 60.138 | 13.261 | 144.239 | 1.00 | 19.36 | B | O |
| ATOM | 1416 | N | TYR B | 125 | 61.164 | 11.666 | 143.016 | 1.00 | 18.73 | B | N |
| ATOM | 1417 | CA | TYR B | 125 | 60.670 | 12.187 | 141.749 | 1.00 | 14.58 | B | C |
| ATOM | 1418 | CB | TYR B | 125 | 61.844 | 12.731 | 140.921 | 1.00 | 15.39 | B | C |
| ATOM | 1419 | CG | TYR B | 125 | 62.538 | 13.932 | 141.536 | 1.00 | 13.70 | B | C |
| ATOM | 1420 | CD1 | TYR B | 125 | 63.526 | 13.777 | 142.510 | 1.00 | 12.60 | B | C |
| ATOM | 1421 | CE1 | TYR B | 125 | 64.113 | 14.889 | 143.124 | 1.00 | 11.44 | B | C |
| ATOM | 1422 | CD2 | TYR B | 125 | 62.156 | 15.229 | 141.187 | 1.00 | 15.12 | B | C |
| ATOM | 1423 | CE2 | TYR B | 125 | 62.731 | 16.341 | 141.793 | 1.00 | 11.78 | B | C |
| ATOM | 1424 | CZ | TYR B | 125 | 63.706 | 16.164 | 142.760 | 1.00 | 13.63 | B | C |
| ATOM | 1425 | OH | TYR B | 125 | 64.257 | 17.270 | 143.365 | 1.00 | 11.62 | B | O |
| ATOM | 1426 | C | TYR B | 125 | 59.890 | 11.203 | 140.899 | 1.00 | 14.07 | B | C |
| ATOM | 1427 | O | TYR B | 125 | 59.880 | 10.005 | 141.158 | 1.00 | 12.64 | B | O |
| ATOM | 1428 | N | SER B | 126 | 59.230 | 11.737 | 139.877 | 1.00 | 15.52 | B | N |
| ATOM | 1429 | CA | SER B | 126 | 58.461 | 10.929 | 138.936 | 1.00 | 17.11 | B | C |
| ATOM | 1430 | CB | SER B | 126 | 57.246 | 11.705 | 138.419 | 1.00 | 17.11 | B | C |
| ATOM | 1431 | OG | SER B | 126 | 56.716 | 11.100 | 137.252 | 1.00 | 14.91 B | O | |
| ATOM | 1432 | C | SER B | 126 | 59.374 | 10.596 | 137.766 | 1.00 | 20.19 | B | C |
| ATOM | 1433 | O | SER B | 126 | 60.298 | 11.345 | 137.452 | 1.00 | 19.00 | B | O |
| ATOM | 1434 | N | GLU B | 127 | 59.109 | 9.480 | 137.108 | 1.00 | 23.95 | B | N |
| ATOM | 1435 | CA | GLU B | 127 | 59.937 | 9.079 | 135.982 | 1.00 | 27.98 | B | C |
| ATOM | 1436 | CB | GLU B | 127 | 59.795 | 7.575 | 135.743 | 1.00 | 33.47 | B | C |
| ATOM | 1437 | CG | GLU B | 127 | 59.913 | 6.740 | 137.004 | 1.00 | 40.62 | B | C |
| ATOM | 1438 | CD | GLU B | 127 | 60.077 | 5.255 | 136.718 | 1.00 | 45.97 | B | C |
| ATOM | 1439 | OE1 | GLU B | 127 | 61.082 | 4.880 | 136.068 | 1.00 | 47.09 | B | O |
| ATOM | 1440 | OE2 | GLU B | 127 | 59.204 | 4.466 | 137.148 | 1.00 | 47.02 | B | O |
| ATOM | 1441 | C | GLU B | 127 | 59.585 | 9.825 | 134.700 | 1.00 | 26.19 | B | C |
| ATOM | 1442 | O | GLU B | 127 | 60.331 | 9.763 | 133.732 | 1.00 | 27.48 | B | O |
| ATOM | 1443 | N | ASP B | 128 | 58.464 | 10.543 | 134.702 | 1.00 | 23.99 | B | N |
| ATOM | 1444 | CA | ASP B | 128 | 58.008 | 11.256 | 133.511 | 1.00 | 21.74 | B | C |
| ATOM | 1445 | CB | ASP B | 128 | 56.496 | 11.480 | 133.557 | 1.00 | 22.01 | B | C |
| ATOM | 1446 | CG | ASP B | 128 | 55.717 | 10.228 | 133.884 | 1.00 | 18.40 | B | C |
| ATOM | 1447 | OD1 | ASP B | 128 | 56.138 | 9.119 | 133.514 | 1.00 | 21.04 | B | O |
| ATOM | 1448 | OD2 | ASP B | 128 | 54.652 | 10.371 | 134.504 | 1.00 | 17.97 | B | O |
| ATOM | 1449 | C | ASP B | 128 | 58.637 | 12.606 | 133.194 | 1.00 | 22.03 | B | C |
| ATOM | 1450 | O | ASP B | 128 | 58.578 | 13.052 | 132.051 | 1.00 | 21.61 | B | O |
| ATOM | 1451 | N | LEU B | 129 | 59.207 | 13.273 | 134.189 | 1.00 | 19.22 | B | N |
| ATOM | 1452 | CA | LEU B | 129 | 59.797 | 14.582 | 133.953 | 1.00 | 17.22 | B | C |
| ATOM | 1453 | CB | LEU B | 129 | 58.887 | 15.664 | 134.528 | 1.00 | 16.52 | B | C |
| ATOM | 1454 | CG | LEU B | 129 | 57.497 | 15.796 | 133.901 | 1.00 | 20.26 | B | C |
| ATOM | 1455 | CD1 | LEU B | 129 | 56.642 | 16.745 | 134.732 | 1.00 | 18.39 | B | C |
| ATOM | 1456 | CD2 | LEU B | 129 | 57.629 | 16.303 | 132.471 | 1.00 | 19.31 | B | C |
| ATOM | 1457 | C | LEU B | 129 | 61.179 | 14.713 | 134.566 | 1.00 | 17.81 | B | C |
| ATOM | 1458 | O | LEU B | 129 | 61.515 | 14.000 | 135.511 | 1.00 | 20.83 | B | O |
| ATOM | 1459 | N | PRO B | 130 | 62.014 | 15.617 | 134.021 | 1.00 | 17.25 | B | N |
| ATOM | 1460 | CD | PRO B | 130 | 61.811 | 16.408 | 132.796 | 1.00 | 15.36 | B | C |
| ATOM | 1461 | CA | PRO B | 130 | 63.371 | 15.820 | 134.557 | 1.00 | 17.25 | B | C |
| ATOM | 1462 | CB | PRO B | 130 | 64.002 | 16.790 | 133.554 | 1.00 | 14.93 | B | C |
| ATOM | 1463 | CG | PRO B | 130 | 63.214 | 16.559 | 132.292 | 1.00 | 15.96 | B | C |
| ATOM | 1464 | C | PRO B | 130 | 63.250 | 16.450 | 135.954 | 1.00 | 15.64 | B | C |
| ATOM | 1465 | O | PRO B | 130 | 62.245 | 17.096 | 136.250 | 1.00 | 15.44 | B | O |
| ATOM | 1466 | N | ARG B | 131 | 64.259 | 16.265 | 136.805 | 1.00 | 16.42 | B | N |
| ATOM | 1467 | CA | ARG B | 131 | 64.237 | 16.840 | 138.159 | 1.00 | 16.86 | B | C |
| ATOM | 1468 | CB | ARG B | 131 | 65.551 | 16.591 | 138.906 | 1.00 | 18.20 | B | C |
| ATOM | 1469 | CG | ARG B | 131 | 65.708 | 15.207 | 139.469 | 1.00 | 22.57 | B | C |
| ATOM | 1470 | CD | ARG B | 131 | 66.845 | 15.143 | 140.470 | 1.00 | 21.69 | B | C |
| ATOM | 1471 | NE | ARG B | 131 | 67.022 | 13.771 | 140.932 | 1.00 | 26.97 | B | N |
| ATOM | 1472 | CZ | ARG B | 131 | 67.541 | 13.427 | 142.107 | 1.00 | 28.81 | B | C |
| ATOM | 1473 | NH1 | ARG B | 131 | 67.945 | 14.360 | 142.966 | 1.00 | 24.33 | B | N |
| ATOM | 1474 | NH2 | ARG B | 131 | 67.641 | 12.143 | 142.424 | 1.00 | 28.78 | B | N |
| ATOM | 1475 | C | ARG B | 131 | 64.022 | 18.338 | 138.138 | 1.00 | 16.07 | B | C |
| ATOM | 1476 | O | ARG B | 131 | 63.219 | 18.869 | 138.899 | 1.00 | 15.45 | B | O |
| ATOM | 1477 | N | ALA B | 132 | 64.764 | 19.013 | 137.268 | 1.00 | 14.39 | B | N |
| ATOM | 1478 | CA | ALA B | 132 | 64.685 | 20.461 | 137.163 | 1.00 | 16.40 | B | C |
| ATOM | 1479 | CB | ALA B | 132 | 65.677 | 20.959 | 136.106 | 1.00 | 17.38 | B | C |
| ATOM | 1480 | C | ALA B | 132 | 63.278 | 20.946 | 136.837 | 1.00 | 16.67 | B | C |
| ATOM | 1481 | O | ALA B | 132 | 62.844 | 21.984 | 137.326 | 1.00 | 18.93 | B | O |
| ATOM | 1482 | N | VAL B | 133 | 62.566 | 20.192 | 136.008 | 1.00 | 15.47 | B | N |
| ATOM | 1483 | CA | VAL B | 133 | 61.213 | 20.562 | 135.626 | 1.00 | 13.65 | B | C |
| ATOM | 1484 | CB | VAL B | 133 | 60.721 | 19.694 | 134.433 | 1.00 | 14.12 | B | C |
| ATOM | 1485 | CG1 | VAL B | 133 | 59.229 | 19.926 | 134.177 | 1.00 | 9.73 | B | C |
| ATOM | 1486 | CG2 | VAL B | 133 | 61.530 | 20.037 | 133.190 | 1.00 | 12.36 | B | C |
| ATOM | 1487 | C | VAL B | 133 | 60.259 | 20.394 | 136.803 | 1.00 | 14.22 | B | C |
| ATOM | 1488 | O | VAL B | 133 | 59.362 | 21.211 | 137.016 | 1.00 | 13.15 | B | O |
| ATOM | 1489 | N | ILE B | 134 | 60.460 | 19.323 | 137.563 | 1.00 | 15.06 | B | N |
| ATOM | 1490 | CA | ILE B | 134 | 59.617 | 19.029 | 138.709 | 1.00 | 13.17 | B | C |
| ATOM | 1491 | CB | ILE B | 134 | 59.855 | 17.580 | 139.200 | 1.00 | 14.87 | B | C |
| ATOM | 1492 | CG2 | ILE B | 134 | 59.229 | 17.371 | 140.571 | 1.00 | 14.89 | B | C |
| ATOM | 1493 | CG1 | ILE B | 134 | 59.287 | 16.597 | 138.166 | 1.00 | 11.36 | B | C |
| ATOM | 1494 | CD1 | ILE B | 134 | 59.476 | 15.131 | 138.521 | 1.00 | 11.57 | B | C |
| ATOM | 1495 | C | ILE B | 134 | 59.862 | 20.025 | 139.830 | 1.00 | 15.99 | B | C |
| ATOM | 1496 | O | ILE B | 134 | 58.908 | 20.543 | 140.404 | 1.00 | 16.24 | B | O |
| ATOM | 1497 | N | ASP B | 135 | 61.132 | 20.308 | 140.134 | 1.00 | 15.66 | B | N |
| ATOM | 1498 | CA | ASP B | 135 | 61.447 | 21.279 | 141.184 | 1.00 | 15.15 | B | C |
| ATOM | 1499 | CB | ASP B | 135 | 62.959 | 21.544 | 141.288 | 1.00 | 12.56 | B | C |
| ATOM | 1500 | CG | ASP B | 135 | 63.746 | 20.351 | 141.803 | 1.00 | 10.35 | B | C |
| ATOM | 1501 | OD1 | ASP B | 135 | 63.156 | 19.399 | 142.353 | 1.00 | 11.63 | B | O |
| ATOM | 1502 | OD2 | ASP B | 135 | 64.983 | 20.371 | 141.664 | 1.00 | 17.25 | B | O |
| ATOM | 1503 | C | ASP B | 135 | 60.752 | 22.603 | 140.871 | 1.00 | 15.48 | B | C |
| ATOM | 1504 | O | ASP B | 135 | 60.095 | 23.187 | 141.732 | 1.00 | 20.45 | B | O |
| ATOM | 1505 | N | ASP B | 136 | 60.902 | 23.069 | 139.635 | 1.00 | 14.76 | B | N |
| ATOM | 1506 | CA | ASP B | 136 | 60.313 | 24.328 | 139.189 | 1.00 | 13.88 | B | C |
| ATOM | 1507 | CB | ASP B | 136 | 60.768 | 24.621 | 137.762 | 1.00 | 16.77 | B | C |
| ATOM | 1508 | CG | ASP B | 136 | 60.207 | 25.919 | 137.221 | 1.00 | 19.24 | B | C |
| ATOM | 1509 | OD1 | ASP B | 136 | 60.611 | 26.985 | 137.721 | 1.00 | 20.16 | B | O |
| ATOM | 1510 | OD2 | ASP B | 136 | 59.362 | 25.870 | 136.297 | 1.00 | 18.28 | B | O |
| ATOM | 1511 | C | ASP B | 136 | 58.785 | 24.354 | 139.267 | 1.00 | 13.68 | B | C |
| ATOM | 1512 | O | ASP B | 136 | 58.200 | 25.343 | 139.697 | 1.00 | 18.58 | B | O |
| ATOM | 1513 | N | ALA B | 137 | 58.135 | 23.276 | 138.850 | 1.00 | 12.86 | B | N |
| ATOM | 1514 | CA | ALA B | 137 | 56.679 | 23.204 | 138.907 | 1.00 | 10.91 | B | C |
| ATOM | 1515 | CB | ALA B | 137 | 56.196 | 21.882 | 138.315 | 1.00 | 9.57 | B | C |
| ATOM | 1516 | C | ALA B | 137 | 56.174 | 23.343 | 140.347 | 1.00 | 13.69 | B | C |
| ATOM | 1517 | O | ALA B | 137 | 55.184 | 24.034 | 140.601 | 1.00 | 14.16 | B | O |
| ATOM | 1518 | N | PHE B | 138 | 56.831 | 22.668 | 141.289 | 1.00 | 15.83 | B | N |
| ATOM | 1519 | CA | PHE B | 138 | 56.425 | 22.758 | 142.694 | 1.00 | 16.29 | B | C |
| ATOM | 1520 | CB | PHE B | 138 | 57.182 | 21.737 | 143.559 | 1.00 | 13.24 | B | C |
| ATOM | 1521 | CG | PHE B | 138 | 56.731 | 20.304 | 143.363 | 1.00 | 12.26 | B | C |
| ATOM | 1522 | CD1 | PHE B | 138 | 55.409 | 20.007 | 143.025 | 1.00 | 13.72 | B | C |
| ATOM | 1523 | CD2 | PHE B | 138 | 57.628 | 19.251 | 143.516 | 1.00 | 9.81 | B | C |
| ATOM | 1524 | CE1 | PHE B | 138 | 54.995 | 18.679 | 142.841 | 1.00 | 13.46 | B | C |
| ATOM | 1525 | CE2 | PHE B | 138 | 57.224 | 17.927 | 143.337 | 1.00 | 7.35 | B | C |
| ATOM | 1526 | CZ | PHE B | 138 | 55.907 | 17.641 | 142.998 | 1.00 | 11.80 | B | C |
| ATOM | 1527 | C | PHE B | 138 | 56.670 | 24.172 | 143.226 | 1.00 | 16.78 | B | C |
| ATOM | 1528 | O | PHE B | 138 | 55.871 | 24.709 | 143.990 | 1.00 | 19.28 | B | O |
| ATOM | 1529 | N | ALA B | 139 | 57.772 | 24.783 | 142.807 | 1.00 | 17.38 | B | N |
| ATOM | 1530 | CA | ALA B | 139 | 58.093 | 26.136 | 143.246 | 1.00 | 17.57 | B | C |
| ATOM | 1531 | CB | ALA B | 139 | 59.490 | 26.521 | 142.770 | 1.00 | 14.41 | B | C |
| ATOM | 1532 | C | ALA B | 139 | 57.061 | 27.149 | 142.738 | 1.00 | 18.95 | B | C |
| ATOM | 1533 | O | ALA B | 139 | 56.664 | 28.060 | 143.467 | 1.00 | 19.80 | B | O |
| ATOM | 1534 | N | ARG B | 140 | 56.628 | 26.993 | 141.490 | 1.00 | 19.15 | B | N |
| ATOM | 1535 | CA | ARG B | 140 | 55.642 | 27.906 | 140.917 | 1.00 | 17.98 | B | C |
| ATOM | 1536 | CE | ARG B | 140 | 55.596 | 27.761 | 139.388 | 1.00 | 16.03 | B | C |
| ATOM | 1537 | CG | ARG B | 140 | 56.888 | 28.201 | 138.707 | 1.00 | 16.18 | B | C |
| ATOM | 1538 | CD | ARG B | 140 | 56.812 | 28.138 | 137.181 | 1.00 | 18.32 | B | C |
| ATOM | 1539 | NE | ARG B | 140 | 56.746 | 26.768 | 136.685 | 1.00 | 14.27 | B | N |
| ATOM | 1540 | CZ | ARG B | 140 | 55.666 | 26.213 | 136.150 | 1.00 | 14.50 | B | C |
| ATOM | 1541 | NH1 | ARG B | 140 | 54.548 | 26.912 | 136.030 | 1.00 | 12.39 | B | N |
| ATOM | 1542 | NH2 | ARG B | 140 | 55.701 | 24.946 | 135.753 | 1.00 | 14.50 | B | N |
| ATOM | 1543 | C | ARG B | 140 | 54.274 | 27.638 | 141.531 | 1.00 | 18.29 | B | C |
| ATOM | 1544 | O | ARG B | 140 | 53.451 | 28.544 | 141.661 | 1.00 | 22.02 | B | O |
| ATOM | 1545 | N | ALA B | 141 | 54.036 | 26.392 | 141.927 | 1.00 | 17.80 | B | N |
| ATOM | 1546 | CA | ALA B | 141 | 52.768 | 26.030 | 142.552 | 1.00 | 17.21 | B | C |
| ATOM | 1547 | CB | ALA B | 141 | 52.656 | 24.520 | 142.660 | 1.00 | 17.90 | B | C |
| ATOM | 1548 | C | ALA B | 141 | 52.660 | 26.677 | 143.941 | 1.00 | 19.87 | B | C |
| ATOM | 1549 | O | ALA B | 141 | 51.572 | 27.056 | 144.369 | 1.00 | 19.56 | B | O |
| ATOM | 1550 | N | PHE B | 142 | 53.787 | 26.795 | 144.645 | 1.00 | 20.99 | B | N |
| ATOM | 1551 | CA | PHE B | 142 | 53.798 | 27.427 | 145.970 | 1.00 | 21.67 | B | C |
| ATOM | 1552 | CB | PHE B | 142 | 55.072 | 27.069 | 146.759 | 1.00 | 18.85 | B | C |
| ATOM | 1553 | CG | PHE B | 142 | 55.008 | 25.734 | 147.446 | 1.00 | 16.57 | B | C |
| ATOM | 1554 | CD1 | PHE B | 142 | 53.976 | 25.449 | 148.333 | 1.00 | 14.21 | B | C |
| ATOM | 1555 | CD2 | PHE B | 142 | 55.974 | 24.764 | 147.204 | 1.00 | 14.86 | B | C |
| ATOM | 1556 | CE1 | PHE B | 142 | 53.904 | 24.220 | 148.972 | 1.00 | 18.27 | B | C |
| ATOM | 1557 | CE2 | PHE B | 142 | 55.917 | 23.527 | 147.836 | 1.00 | 19.57 | B | C |
| ATOM | 1558 | CZ | PHE B | 142 | 54.879 | 23.251 | 148.724 | 1.00 | 20.55 | B | C |
| ATOM | 1559 | C | PHE B | 142 | 53.719 | 28.942 | 145.832 | 1.00 | 21.95 | B | C |
| ATOM | 1560 | O | PHE B | 142 | 53.101 | 29.613 | 146.652 | 1.00 | 25.86 | B | O |
| ATOM | 1561 | N | ALA B | 143 | 54.356 | 29.481 | 144.802 | 1.00 | 20.87 | B | N |
| ATOM | 1562 | CA | ALA B | 143 | 54.344 | 30.921 | 144.576 | 1.00 | 21.45 | B | C |
| ATOM | 1563 | CB | ALA B | 143 | 55.222 | 31.268 | 143.390 | 1.00 | 17.43 | B | C |
| ATOM | 1564 | C | ALA B | 143 | 52.930 | 31.466 | 144.360 | 1.00 | 23.06 | B | C |
| ATOM | 1565 | O | ALA B | 143 | 52.661 | 32.629 | 144.658 | 1.00 | 25.48 | B | O |
| ATOM | 1566 | N | LEU B | 144 | 52.029 | 30.636 | 143.841 | 1.00 | 24.74 | B | N |
| ATOM | 1567 | CA | LEU B | 144 | 50.652 | 31.075 | 143.615 | 1.00 | 26.04 | B | C |
| ATOM | 1568 | CB | LEU B | 144 | 49.797 | 29.958 | 143.010 | 1.00 | 24.21 | B | C |
| ATOM | 1569 | CG | LEU B | 144 | 49.951 | 29.560 | 141.539 | 1.00 | 27.50 | B | C |
| ATOM | 1570 | CD1 | LEU B | 144 | 48.945 | 28.468 | 141.230 | 1.00 | 25.16 | B | C |
| ATOM | 1571 | CD2 | LEU B | 144 | 49.718 | 30.754 | 140.630 | 1.00 | 23.79 | B | C |
| ATOM | 1572 | C | LEU B | 144 | 50.020 | 31.481 | 144.934 | 1.00 | 28.47 | B | C |
| ATOM | 1573 | O | LEU B | 144 | 49.343 | 32.500 | 145.030 | 1.00 | 30.66 | B | O |
| ATOM | 1574 | N | TRP B | 145 | 50.245 | 30.664 | 145.951 | 1.00 | 28.49 | B | N |
| ATOM | 1575 | CA | TRP B | 145 | 49.680 | 30.913 | 147.265 | 1.00 | 29.54 | B | C |
| ATOM | 1576 | CB | TRP B | 145 | 49.652 | 29.609 | 148.053 | 1.00 | 26.39 | B | C |
| ATOM | 1577 | CG | TRP B | 145 | 48.796 | 28.553 | 147.417 | 1.00 | 24.57 | B | C |
| ATOM | 1578 | CD2 | TRP B | 145 | 47.366 | 28.550 | 147.330 | 1.00 | 25.64 | B | C |
| ATOM | 1579 | CE2 | TRP B | 145 | 46.986 | 27.320 | 146.747 | 1.00 | 24.80 | B | C |
| ATOM | 1580 | CR3 | TRP B | 145 | 46.366 | 29.464 | 147.695 | 1.00 | 25.42 | B | C |
| ATOM | 1581 | CD1 | TRP B | 145 | 49.214 | 27.369 | 146.885 | 1.00 | 22.40 | B | C |
| ATOM | 1582 | NR1 | TRP B | 145 | 48.133 | 26.619 | 146.483 | 1.00 | 22.24 | B | N |
| ATOM | 1583 | CZ2 | TRP B | 145 | 45.644 | 26.977 | 146.522 | 1.00 | 27.07 | B | C |
| ATOM | 1584 | CZ3 | TRP B | 145 | 45.029 | 29.122 | 147.471 | 1.00 | 26.11 | B | C |
| ATOM | 1585 | CH2 | TRP B | 145 | 44.684 | 27.889 | 146.892 | 1.00 | 24.97 | B | C |
| ATOM | 1586 | C | TRP B | 145 | 50.397 | 31.995 | 148.064 | 1.00 | 28.97 | B | C |
| ATOM | 1587 | O | TRP B | 145 | 49.752 | 32.844 | 148.674 | 1.00 | 29.64 | B | O |
| ATOM | 1588 | N | SER B | 146 | 51.724 | 31.979 | 148.063 | 1.00 | 30.94 | B | N |
| ATOM | 1589 | CA | SER B | 146 | 52.474 | 32.986 | 148.808 | 1.00 | 33.11 | B | C |
| ATOM | 1590 | CB | SER B | 146 | 53.967 | 32.675 | 148.776 | 1.00 | 31.16 | B | C |
| ATOM | 1591 | OG | SER B | 146 | 54.465 | 32.773 | 147.457 | 1.00 | 34.33 | B | O |
| ATOM | 1592 | C | SER B | 146 | 52.241 | 34.372 | 148.221 | 1.00 | 35.39 | B | C |
| ATOM | 1593 | O | SER B | 146 | 52.600 | 35.381 | 148.827 | 1.00 | 38.15 | B | O |
| ATOM | 1594 | N | ALA B | 147 | 51.641 | 34.418 | 147.039 | 1.00 | 35.46 | B | N |
| ATOM | 1595 | CA | ALA B | 147 | 51.379 | 35.683 | 146.374 | 1.00 | 35.64 | B | C |
| ATOM | 1596 | CB | ALA B | 147 | 51.187 | 35.457 | 144.883 | 1.00 | 35.78 | B | C |
| ATOM | 1597 | C | ALA B | 147 | 50.160 | 36.380 | 146.949 | 1.00 | 36.30 | B | C |
| ATOM | 1598 | O | ALA B | 147 | 50.054 | 37.600 | 146.875 | 1.00 | 35.93 | B | O |
| ATOM | 1599 | N | VAL B | 148 | 49.238 | 35.611 | 147.519 | 1.00 | 36.26 | B | N |
| ATOM | 1600 | CA | VAL B | 148 | 48.031 | 36.196 | 148.089 | 1.00 | 37.29 | B | C |
| ATOM | 1601 | CB | VAL B | 148 | 46.761 | 35.601 | 147.435 | 1.00 | 37.45 | B | C |
| ATOM | 1602 | CG1 | VAL B | 148 | 46.728 | 35.952 | 145.959 | 1.00 | 39.35 | B | C |
| ATOM | 1603 | CG2 | VAL B | 148 | 46.735 | 34.099 | 147.614 | 1.00 | 37.42 | B | C |
| ATOM | 1604 | C | VAL B | 148 | 47.926 | 36.035 | 149.606 | 1.00 | 37.54 | B | C |
| ATOM | 1605 | O | VAL B | 148 | 46.838 | 36.144 | 150.173 | 1.00 | 38.76 | B | O |
| ATOM | 1606 | N | THR B | 149 | 49.057 | 35.774 | 150.257 | 1.00 | 36.72 | B | N |
| ATOM | 1607 | CA | THR B | 149 | 49.098 | 35.611 | 151.711 | 1.00 | 34.50 | B | C |
| ATOM | 1608 | CB | THR B | 149 | 48.815 | 34.159 | 152.139 | 1.00 | 32.69 | B | C |
| ATOM | 1609 | OG1 | THR B | 149 | 49.872 | 33.313 | 151.672 | 1.00 | 34.10 | B | O |
| ATOM | 1610 | CG2 | THR B | 149 | 47.492 | 33.677 | 151.587 | 1.00 | 33.09 | B | C |
| ATOM | 1611 | C | THR B | 149 | 50.484 | 35.975 | 152.241 | 1.00 | 34.43 | B | C |
| ATOM | 1612 | O | THR B | 149 | 51.424 | 36.166 | 151.466 | 1.00 | 33.86 | B | O |
| ATOM | 1613 | N | PRO B | 150 | 50.624 | 36.084 | 153.576 | 1.00 | 35.88 | B | N |
| ATOM | 1614 | CD | PRO B | 150 | 49.542 | 36.096 | 154.582 | 1.00 | 36.97 | B | C |
| ATOM | 1615 | CA | PRO B | 150 | 51.909 | 36.423 | 154.199 | 1.00 | 35.29 | B | C |
| ATOM | 1616 | CB | PRO B | 150 | 51.491 | 36.993 | 155.548 | 1.00 | 33.79 | B | C |
| ATOM | 1617 | CG | PRO B | 150 | 50.313 | 36.136 | 155.894 | 1.00 | 36.98 | B | C |
| ATOM | 1618 | C | PRO B | 150 | 52.810 | 35.190 | 154.341 | 1.00 | 33.55 | B | C |
| ATOM | 1619 | O | PRO B | 150 | 53.889 | 35.259 | 154.928 | 1.00 | 33.85 | B | O |
| ATOM | 1620 | N | LEU B | 151 | 52.357 | 34.064 | 153.796 | 1.00 | 32.24 | B | N |
| ATOM | 1621 | CA | LEU B | 151 | 53.111 | 32.813 | 153.850 | 1.00 | 29.72 | B | C |
| ATOM | 1622 | CB | LEU B | 151 | 52.210 | 31.634 | 153.485 | 1.00 | 28.34 | B | C |
| ATOM | 1623 | CG | LEU B | 151 | 50.920 | 31.386 | 154.263 | 1.00 | 29.72 | B | C |
| ATOM | 1624 | CD1 | LEU B | 151 | 50.115 | 30.308 | 153.547 | 1.00 | 25.73 | B | C |
| ATOM | 1625 | CD2 | LEU B | 151 | 51.241 | 30.971 | 155.697 | 1.00 | 27.68 | B | C |
| ATOM | 1626 | C | LEU B | 151 | 54.301 | 32.796 | 152.897 | 1.00 | 28.80 | B | C |
| ATOM | 1627 | O | LEU B | 151 | 54.253 | 33.384 | 151.817 | 1.00 | 29.79 | B | O |
| ATOM | 1628 | N | THR B | 152 | 55.370 | 32.123 | 153.307 | 1.00 | 26.47 | B | N |
| ATOM | 1629 | CA | THR B | 152 | 56.550 | 31.968 | 152.468 | 1.00 | 26.48 | B | C |
| ATOM | 1630 | CB | THR B | 152 | 57.786 | 32.697 | 153.018 | 1.00 | 25.05 | B | C |
| ATOM | 1631 | OG1 | THR B | 152 | 58.199 | 32.092 | 154.250 | 1.00 | 26.56 | B | O |
| ATOM | 1632 | CG2 | THR B | 152 | 57.478 | 34.161 | 153.235 | 1.00 | 26.91 | B | C |
| ATOM | 1633 | C | THR B | 152 | 56.822 | 30.469 | 152.444 | 1.00 | 27.63 | B | C |
| ATOM | 1634 | O | THR B | 152 | 56.361 | 29.731 | 153.321 | 1.00 | 28.65 | B | O |
| ATOM | 1635 | N | PHE B | 153 | 57.561 | 30.020 | 151.438 | 1.00 | 26.34 | B | N |
| ATOM | 1636 | CA | PHE B | 153 | 57.858 | 28.603 | 151.289 | 1.00 | 24.22 | B | C |
| ATOM | 1637 | CB | PHE B | 153 | 57.010 | 28.041 | 150.147 | 1.00 | 21.10 | B | C |
| ATOM | 1638 | CG | PHE B | 153 | 55.531 | 28.101 | 150.419 | 1.00 | 21.18 | B | C |
| ATOM | 1639 | CD1 | PHE B | 153 | 54.916 | 27.141 | 151.229 | 1.00 | 19.72 | B | C |
| ATOM | 1640 | CD2 | PHE B | 153 | 54.761 | 29.150 | 149.924 | 1.00 | 18.40 | B | C |
| ATOM | 1641 | CE1 | PHE B | 153 | 53.560 | 27.231 | 151.541 | 1.00 | 18.13 | B | C |
| ATOM | 1642 | CE2 | PHE B | 153 | 53.404 | 29.249 | 150.231 | 1.00 | 15.81 | B | C |
| ATOM | 1643 | CZ | PHE B | 153 | 52.803 | 28.291 | 151.040 | 1.00 | 17.61 | B | C |
| ATOM | 1644 | C | PHE B | 153 | 59.338 | 28.362 | 151.045 | 1.00 | 23.36 | B | C |
| ATOM | 1645 | O | PHE B | 153 | 59.910 | 28.853 | 150.079 | 1.00 | 25.84 | B | O |
| ATOM | 1646 | N | THR B | 154 | 59.951 | 27.596 | 151.938 | 1.00 | 24.03 | B | N |
| ATOM | 1647 | CA | THR B | 154 | 61.375 | 27.302 | 151.858 | 1.00 | 22.52 | B | C |
| ATOM | 1648 | CB | THR B | 154 | 62.075 | 27.739 | 153.149 | 1.00 | 21.34 | B | C |
| ATOM | 1649 | OG1 | THR B | 154 | 61.867 | 29.138 | 153.328 | 1.00 | 25.73 | B | O |
| ATOM | 1650 | CG2 | THR B | 154 | 63.567 | 27.461 | 153.091 | 1.00 | 20.97 | B | C |
| ATOM | 1651 | C | THR B | 154 | 61.629 | 25.824 | 151.638 | 1.00 | 21.11 | B | C |
| ATOM | 1652 | O | THR B | 154 | 61.050 | 24.977 | 152.317 | 1.00 | 21.31 | B | O |
| ATOM | 1653 | N | ARG B | 155 | 62.502 | 25.521 | 150.688 | 1.00 | 21.15 | B | N |
| ATOM | 1654 | CA | ARG B | 155 | 62.831 | 24.138 | 150.385 | 1.00 | 21.88 | B | C |
| ATOM | 1655 | CB | ARG B | 155 | 63.248 | 23.996 | 148.919 | 1.00 | 23.11 | B | C |
| ATOM | 1656 | CG | ARG B | 155 | 63.532 | 22.559 | 148.524 | 1.00 | 20.37 | B | C |
| ATOM | 1657 | CD | ARG B | 155 | 64.052 | 22.459 | 147.121 | 1.00 | 21.66 | B | C |
| ATOM | 1658 | NE | ARG B | 155 | 64.381 | 21.083 | 146.772 | 1.00 | 18.52 | B | N |
| ATOM | 1659 | CZ | ARG B | 155 | 64.905 | 20.721 | 145.610 | 1.00 | 17.45 | B | C |
| ATOM | 1660 | NH1 | ARG B | 155 | 65.158 | 21.637 | 144.689 | 1.00 | 16.66 | B | N |
| ATOM | 1661 | NH2 | ARG B | 155 | 65.174 | 19.447 | 145.372 | 1.00 | 15.63 | B | N |
| ATOM | 1662 | C | ARG B | 155 | 63.963 | 23.657 | 151.284 | 1.00 | 22.35 | B | C |
| ATOM | 1663 | O | ARG B | 155 | 64.973 | 24.341 | 151.435 | 1.00 | 23.68 | B | O |
| ATOM | 1664 | N | VAL B | 156 | 63.784 | 22.486 | 151.888 | 1.00 | 21.22 | B | N |
| ATOM | 1665 | CA | VAL B | 156 | 64.795 | 21.904 | 152.760 | 1.00 | 21.38 | B | C |
| ATOM | 1666 | CB | VAL B | 156 | 64.384 | 22.006 | 154.252 | 1.00 | 21.61 | B | C |
| ATOM | 1667 | CG1 | VAL B | 156 | 64.096 | 23.462 | 154.600 | 1.00 | 19.88 | B | C |
| ATOM | 1668 | CG2 | VAL B | 156 | 63.158 | 21.135 | 154.537 | 1.00 | 20.58 | B | C |
| ATOM | 1669 | C | VAL B | 156 | 64.951 | 20.447 | 152.345 | 1.00 | 23.15 | B | C |
| ATOM | 1670 | O | VAL B | 156 | 64.240 | 19.988 | 151.456 | 1.00 | 23.36 | B | O |
| ATOM | 1671 | N | TYR B | 157 | 65.864 | 19.717 | 152.982 | 1.00 | 24.51 | B | N |
| ATOM | 1672 | CA | TYR B | 157 | 66.100 | 18.321 | 152.613 | 1.00 | 25.70 | B | C |
| ATOM | 1673 | CB | TYR B | 157 | 67.475 | 18.190 | 151.956 | 1.00 | 23.43 | B | C |
| ATOM | 1674 | OG | TYR B | 157 | 67.634 | 19.050 | 150.728 | 1.00 | 23.94 | B | C |
| ATOM | 1675 | OD1 | TYR B | 157 | 67.800 | 20.430 | 150.837 | 1.00 | 22.81 | B | C |
| ATOM | 1676 | CE1 | TYR B | 157 | 67.891 | 21.233 | 149.703 | 1.00 | 24.82 | B | C |
| ATOM | 1677 | CD2 | TYR B | 157 | 67.567 | 18.492 | 149.451 | 1.00 | 24.09 | B | C |
| ATOM | 1678 | CE2 | TYR B | 157 | 67.658 | 19.285 | 148.313 | 1.00 | 24.05 | B | C |
| ATOM | 1679 | CZ | TYR B | 157 | 67.820 | 20.652 | 148.445 | 1.00 | 25.74 | B | C |
| ATOM | 1680 | OH | TYR B | 157 | 67.919 | 21.438 | 147.319 | 1.00 | 32.12 | B | O |
| ATOM | 1681 | C | TYR B | 157 | 65.991 | 17.297 | 153.735 | 1.00 | 27.77 | B | C |
| ATOM | 1682 | O | TYR B | 157 | 66.644 | 16.254 | 153.696 | 1.00 | 28.36 | B | O |
| ATOM | 1683 | N | SER B | 158 | 65.162 | 17.576 | 154.731 | 1.00 | 29.96 | B | N |
| ATOM | 1684 | CA | SER B | 158 | 65.006 | 16.641 | 155.834 | 1.00 | 32.14 | B | C |
| ATOM | 1685 | CE | SER B | 158 | 65.751 | 17.144 | 157.077 | 1.00 | 32.84 | B | C |
| ATOM | 1686 | OG | SER B | 158 | 65.274 | 18.414 | 157.490 | 1.00 | 34.79 | B | O |
| ATOM | 1687 | C | SER B | 158 | 63.549 | 16.412 | 156.178 | 1.00 | 33.22 | B | C |
| ATOM | 1688 | O | SER B | 158 | 62.667 | 17.155 | 155.750 | 1.00 | 29.83 | B | O |
| ATOM | 1689 | N | ARG B | 159 | 63.319 | 15.376 | 156.973 | 1.00 | 37.03 | B | N |
| ATOM | 1690 | CA | ARG B | 159 | 61.990 | 14.999 | 157.418 | 1.00 | 40.29 | B | C |
| ATOM | 1691 | CB | ARG B | 159 | 62.071 | 13.676 | 158.176 | 1.00 | 46.56 | B | C |
| ATOM | 1692 | CC | ARG B | 159 | 60.957 | 12.699 | 157.863 | 1.00 | 57.91 | B | C |
| ATOM | 1693 | CD | ARG B | 159 | 61.049 | 11.467 | 158.757 | 1.00 | 64.70 | B | C |
| ATOM | 1694 | NE | ARG B | 159 | 59.974 | 10.514 | 158.490 | 1.00 | 70.47 | B | N |
| ATOM | 1695 | CZ | ARG B | 159 | 59.694 | 9.466 | 159.260 | 1.00 | 73.51 | B | C |
| ATOM | 1696 | NH1 | ARG B | 159 | 58.698 | 8.651 | 158.937 | 1.00 | 74.22 | B | N |
| ATOM | 1697 | NH2 | ARG B | 159 | 60.406 | 9.237 | 160.357 | 1.00 | 74.50 | B | N |
| ATOM | 1698 | C | ARG B | 159 | 61.395 | 16.078 | 158.325 | 1.00 | 38.61 | B | C |
| ATOM | 1699 | O | ARG B | 159 | 60.264 | 15.949 | 158.781 | 1.00 | 39.20 | B | O |
| ATOM | 1700 | N | ASP B | 160 | 62.160 | 17.135 | 158.583 | 1.00 | 37.90 | B | N |
| ATOM | 1701 | CA | ASP B | 160 | 61.707 | 18.233 | 159.438 | 1.00 | 36.97 | B | C |
| ATOM | 1702 | CB | ASP B | 160 | 62.894 | 19.043 | 159.971 | 1.00 | 42.42 | B | C |
| ATOM | 1703 | CG | ASP B | 160 | 63.908 | 18.195 | 160.698 | 1.00 | 46.87 | B | C |
| ATOM | 1704 | OD1 | ASP B | 160 | 63.505 | 17.346 | 161.523 | 1.00 | 49.44 | B O | |
| ATOM | 1705 | OD2 | ASP B | 160 | 65.116 | 18.393 | 160.451 | 1.00 | 50.63 | B | O |
| ATOM | 1706 | C | ASP B | 160 | 60.784 | 19.198 | 158.706 | 1.00 | 33.52 | B | C |
| ATOM | 1707 | O | ASP B | 160 | 60.165 | 20.065 | 159.326 | 1.00 | 33.69 | B | O |
| ATOM | 1708 | N | ALA B | 161 | 60.706 | 19.065 | 157.389 | 1.00 | 29.73 | B | N |
| ATOM | 1709 | CA | ALA B | 161 | 59.864 | 19.948 | 156.594 | 1.00 | 25.02 | B | C |
| ATOM | 1710 | CB | ALA B | 161 | 60.019 | 19.620 | 155.116 | 1.00 | 24.48 | B | C |
| ATOM | 1711 | C | ALA B | 161 | 58.408 | 19.808 | 157.005 | 1.00 | 22.73 | B | C |
| ATOM | 1712 | O | ALA B | 161 | 57.983 | 18.743 | 157.453 | 1.00 | 18.59 | B O | |
| ATOM | 1713 | N | ASP B | 162 | 57.644 | 20.886 | 156.865 | 1.00 | 20.01 | B | N |
| ATOM | 1714 | CA | ASP B | 162 | 56.226 | 20.835 | 157.207 | 1.00 | 19.49 | B | C |
| ATOM | 1715 | CB | ASP B | 162 | 55.625 | 22.240 | 157.298 | 1.00 | 17.17 | B | C |
| ATOM | 1716 | CG | ASP B | 162 | 56.279 | 23.087 | 158.365 | 1.00 | 15.31 | B | C |
| ATOM | 1717 | OD1 | ASP B | 162 | 56.422 | 22.601 | 159.505 | 1.00 | 16.45 | B | O |
| ATOM | 1718 | OD2 | ASP B | 162 | 56.643 | 24.242 | 158.066 | 1.00 | 14.81 | B | O |
| ATOM | 1719 | C | ASP B | 162 | 55.486 | 20.055 | 156.128 | 1.00 | 20.86 | B | C |
| ATOM | 1720 | O | ASP B | 162 | 54.620 | 19.240 | 156.417 | 1.00 | 19.12 | B | O |
| ATOM | 1721 | N | ILE B | 163 | 55.836 | 20.312 | 154.875 | 1.00 | 20.30 | B | N |
| ATOM | 1722 | CA | ILE B | 163 | 55.183 | 19.646 | 153.760 | 1.00 | 17.16 | B | C |
| ATOM | 1723 | CB | ILE B | 163 | 54.640 | 20.692 | 152.748 | 1.00 | 19.96 | B | C |
| ATOM | 1724 | CG2 | ILE B | 163 | 54.032 | 19.995 | 151.530 | 1.00 | 16.75 | B | C |
| ATOM | 1725 | CG1 | ILE B | 163 | 53.611 | 21.587 | 153.449 | 1.00 | 20.77 | B | C |
| ATOM | 1726 | CD1 | ILE B | 163 | 53.139 | 22.769 | 152.625 | 1.00 | 16.61 | B | C |
| ATOM | 1727 | C | ILE B | 163 | 56.138 | 18.694 | 153.058 | 1.00 | 15.84 | B | C |
| ATOM | 1728 | O | ILE B | 163 | 57.096 | 19.114 | 152.411 | 1.00 | 16.45 | B | O |
| ATOM | 1729 | N | VAL B | 164 | 55.883 | 17.405 | 153.207 | 1.00 | 13.42 | B | N |
| ATOM | 1730 | CA | VAL B | 164 | 56.711 | 16.408 | 152.567 | 1.00 | 13.63 | B | C |
| ATOM | 1731 | CB | VAL B | 164 | 56.977 | 15.221 | 153.498 | 1.00 | 13.46 | B | C |
| ATOM | 1732 | CG1 | VAL B | 164 | 57.737 | 14.136 | 152.750 | 1.00 | 11.25 | B | C |
| ATOM | 1733 | CG2 | VAL B | 164 | 57.773 | 15.694 | 154.713 | 1.00 | 12.79 | B | C |
| ATOM | 1734 | C | VAL B | 164 | 55.991 | 15.939 | 151.310 | 1.00 | 14.94 | B | C |
| ATOM | 1735 | O | VAL B | 164 | 54.826 | 15.535 | 151.358 | 1.00 | 15.76 | B | O |
| ATOM | 1736 | N | ILE B | 165 | 56.698 | 16.015 | 150.187 | 1.00 | 13.79 | B | N |
| ATOM | 1737 | CA | ILE B | 165 | 56.165 | 15.636 | 148.886 | 1.00 | 12.71 | B | C |
| ATOM | 1738 | CB | ILE B | 165 | 56.472 | 16.714 | 147.857 | 1.00 | 14.97 | B | C |
| ATOM | 1739 | CG2 | ILE B | 165 | 55.912 | 16.306 | 146.503 | 1.00 | 13.61 | B | C |
| ATOM | 1740 | CG1 | ILE B | 165 | 55.919 | 18.059 | 148.348 | 1.00 | 13.19 | B | C |
| ATOM | 1741 | CD1 | ILE B | 165 | 56.377 | 19.259 | 147.543 | 1.00 | 14.12 | B | C |
| ATOM | 1742 | C | ILE B | 165 | 56.749 | 14.334 | 148.381 | 1.00 | 13.67 | B | C |
| ATOM | 1743 | O | ILE B | 165 | 57.949 | 14.096 | 148.491 | 1.00 | 11.91 | B | O |
| ATOM | 1744 | N | GLN B | 166 | 55.903 | 13.481 | 147.820 | 1.00 | 14.64 | B | N |
| ATOM | 1745 | CA | GLN B | 166 | 56.400 | 12.230 | 147.291 | 1.00 | 17.38 | B | C |
| ATOM | 1746 | CB | GLN B | 166 | 56.599 | 11.209 | 148.412 | 1.00 | 20.52 | B | C |
| ATOM | 1747 | CG | GLN B | 166 | 55.328 | 10.614 | 148.956 | 1.00 | 28.99 | B | C |
| ATOM | 1748 | CD | GLN B | 166 | 55.589 | 9.395 | 149.828 | 1.00 | 34.11 | B | C |
| ATOM | 1749 | OE1 | GLN B | 166 | 55.972 | 8.329 | 149.334 | 1.00 | 34.45 | B | O |
| ATOM | 1750 | NE2 | GLN B | 166 | 55.392 | 9.550 | 151.134 | 1.00 | 36.00 | B | N |
| ATOM | 1751 | C | GLN B | 166 | 55.511 | 11.625 | 146.226 | 1.00 | 16.18 | B | C |
| ATOM | 1752 | O | GLN B | 166 | 54.304 | 11.856 | 146.204 | 1.00 | 14.81 | B | O |
| ATOM | 1753 | N | PHE B | 167 | 56.141 | 10.859 | 145.339 | 1.00 | 14.40 | B | N |
| ATOM | 1754 | CA | PHE B | 167 | 55.463 | 10.147 | 144.274 | 1.00 | 13.46 | B | C |
| ATOM | 1755 | CB | PHE B | 167 | 56.237 | 10.257 | 142.959 | 1.00 | 9.79 | B | C |
| ATOM | 1756 | CG | PHE B | 167 | 56.265 | 11.642 | 142.386 | 1.00 | 11.55 | B | C |
| ATOM | 1757 | CD1 | PHE B | 167 | 57.172 | 12.581 | 142.852 | 1.00 | 12.24 | B | C |
| ATOM | 1758 | CD2 | PHE B | 167 | 55.371 | 12.012 | 141.388 | 1.00 | 12.04 | B | C |
| ATOM | 1759 | CE1 | PHE B | 167 | 57.191 | 13.874 | 142.334 | 1.00 | 12.05 | B | C |
| ATOM | 1760 | CE2 | PHE B | 167 | 55.379 | 13.306 | 140.865 | 1.00 | 11.98 | B | C |
| ATOM | 1761 | CZ | PHE B | 167 | 56.288 | 14.237 | 141.338 | 1.00 | 13.47 | B | C |
| ATOM | 1762 | C | PHE B | 167 | 55.414 | 8.685 | 144.681 | 1.00 | 14.78 | B | C |
| ATOM | 1763 | O | PHE B | 167 | 56.389 | 8.160 | 145.202 | 1.00 | 16.19 | B | O |
| ATOM | 1764 | N | GLY B | 168 | 54.283 | 8.028 | 144.446 | 1.00 | 16.63 | B | N |
| ATOM | 1765 | CA | GLY B | 168 | 54.170 | 6.626 | 144.795 | 1.00 | 18.19 | B | C |
| ATOM | 1766 | C | GLY B | 168 | 53.143 | 5.900 | 143.951 | 1.00 | 20.06 | B | C |
| ATOM | 1767 | O | GLY B | 168 | 52.352 | 6.535 | 143.260 | 1.00 | 21.45 | B | O |
| ATOM | 1768 | N | VAL B | 169 | 53.168 | 4.572 | 143.988 | 1.00 | 21.07 | B | N |
| ATOM | 1769 | CA | VAL B | 169 | 52.206 | 3.764 | 143.247 | 1.00 | 22.74 | B | C |
| ATOM | 1770 | CB | VAL B | 169 | 52.875 | 2.967 | 142.109 | 1.00 | 22.64 | B | C |
| ATOM | 1771 | CG1 | VAL B | 169 | 53.593 | 3.913 | 141.170 | 1.00 | 22.95 | B | C |
| ATOM | 1772 | CG2 | VAL B | 169 | 53.832 | 1.943 | 142.686 | 1.00 | 23.18 | B | C |
| ATOM | 1773 | C | VAL B | 169 | 51.547 | 2.773 | 144.203 | 1.00 | 24.58 | B | C |
| ATOM | 1774 | O | VAL B | 169 | 52.210 | 2.174 | 145.055 | 1.00 | 24.53 | B | O |
| ATOM | 1775 | N | ALA B | 170 | 50.238 | 2.607 | 144.061 | 1.00 | 25.72 | B | N |
| ATOM | 1776 | CA | ALA B | 170 | 49.498 | 1.690 | 144.912 | 1.00 | 25.50 | B | C |
| ATOM | 1777 | CB | ALA B | 170 | 49.845 | 0.256 | 144.543 | 1.00 | 24.40 | B | C |
| ATOM | 1778 | C | ALA B | 170 | 49.826 | 1.959 | 146.379 | 1.00 | 25.00 | B | C |
| ATOM | 1779 | O | ALA B | 170 | 49.761 | 3.106 | 146.836 | 1.00 | 23.10 | B | O |
| ATOM | 1780 | N | GLU B | 171 | 50.179 | 0.902 | 147.105 | 1.00 | 26.18 | B | N |
| ATOM | 1781 | CA | GLU B | 171 | 50.521 | 1.002 | 148.523 | 1.00 | 30.44 | B | C |
| ATOM | 1782 | CB | GLU B | 171 | 50.756 | −0.391 | 149.113 | 1.00 | 37.69 | B | C |
| ATOM | 1783 | CG | GLU B | 171 | 49.929 | −1.502 | 148.484 | 1.00 | 50.32 | B | C |
| ATOM | 1784 | CD | GLU B | 171 | 48.452 | −1.388 | 148.800 | 1.00 | 56.96 | B | C |
| ATOM | 1785 | OE1 | GLU B | 171 | 47.671 | −2.226 | 148.296 | 1.00 | 59.68 | B | O |
| ATOM | 1786 | OE2 | GLU B | 171 | 48.074 | −0.464 | 149.555 | 1.00 | 61.18 | B | O |
| ATOM | 1787 | C | GLU B | 171 | 51.810 | 1.802 | 148.647 | 1.00 | 28.21 | B | C |
| ATOM | 1788 | O | GLU B | 171 | 52.839 | 1.399 | 148.108 | 1.00 | 26.23 | B | O |
| ATOM | 1789 | N | HIS B | 172 | 51.776 | 2.922 | 149.360 | 1.00 | 26.31 | B | N |
| ATOM | 1790 | CA | HIS B | 172 | 52.989 | 3.723 | 149.488 | 1.00 | 26.29 | B | C |
| ATOM | 1791 | CB | HIS B | 172 | 52.935 | 4.898 | 148.500 | 1.00 | 21.05 | B | C |
| ATOM | 1792 | CG | HIS B | 172 | 51.733 | 5.777 | 148.671 | 1.00 | 19.27 | B | C |
| ATOM | 1793 | CD2 | HIS B | 172 | 51.536 | 6.874 | 149.438 | 1.00 | 17.23 | B | C |
| ATOM | 1794 | ND1 | HIS B | 172 | 50.526 | 5.519 | 148.056 | 1.00 | 15.56 | B | N |
| ATOM | 1795 | CE1 | HIS B | 172 | 49.638 | 6.416 | 148.441 | 1.00 | 14.96 | B | C |
| ATOM | 1796 | NE2 | HIS B | 172 | 50.225 | 7.248 | 149.281 | 1.00 | 13.17 | B | N |
| ATOM | 1797 | C | HIS B | 172 | 53.334 | 4.246 | 150.890 | 1.00 | 27.55 | B | C |
| ATOM | 1798 | O | HIS B | 172 | 54.057 | 5.231 | 151.019 | 1.00 | 26.45 | B | O |
| ATOM | 1799 | N | GLY B | 173 | 52.831 | 3.601 | 151.940 | 1.00 | 30.22 | B | N |
| ATOM | 1800 | CA | GLY B | 173 | 53.154 | 4.070 | 153.280 | 1.00 | 32.63 | B | C |
| ATOM | 1801 | C | GLY B | 173 | 51.972 | 4.307 | 154.198 | 1.00 | 35.23 | B | C |
| ATOM | 1802 | O | GLY B | 173 | 51.827 | 3.621 | 155.209 | 1.00 | 39.12 | B | O |
| ATOM | 1803 | N | ASP B | 174 | 51.138 | 5.289 | 153.875 | 1.00 | 33.34 | B | N |
| ATOM | 1804 | CA | ASP B | 174 | 49.968 | 5.563 | 154.692 | 1.00 | 33.01 | B | C |
| ATOM | 1805 | CB | ASP B | 174 | 49.343 | 6.899 | 154.299 | 1.00 | 34.56 | B | C |
| ATOM | 1806 | CG | ASP B | 174 | 49.366 | 7.138 | 152.797 | 1.00 | 33.72 | B | C |
| ATOM | 1807 | OD1 | ASP B | 174 | 49.119 | 6.191 | 152.023 | 1.00 | 30.99 | B | O |
| ATOM | 1808 | OD2 | ASP B | 174 | 49.623 | 8.286 | 152.395 | 1.00 | 32.06 | B | O |
| ATOM | 1809 | C | ASP B | 174 | 48.969 | 4.438 | 154.475 | 1.00 | 33.84 | B | C |
| ATOM | 1810 | O | ASP B | 174 | 49.323 | 3.368 | 153.982 | 1.00 | 35.39 | B | O |
| ATOM | 1811 | N | GLY B | 175 | 47.719 | 4.666 | 154.837 | 1.00 | 34.25 | B | N |
| ATOM | 1812 | CA | GLY B | 175 | 46.732 | 3.620 | 154.641 | 1.00 | 36.75 | B | C |
| ATOM | 1813 | C | GLY B | 175 | 45.836 | 3.952 | 153.474 | 1.00 | 35.04 | B | C |
| ATOM | 1814 | O | GLY B | 175 | 44.705 | 3.478 | 153.390 | 1.00 | 37.61 | B | O |
| ATOM | 1815 | N | TYR B | 176 | 46.359 | 4.768 | 152.566 | 1.00 | 33.73 | B | N |
| ATOM | 1816 | CA | TYR B | 176 | 45.597 | 5.200 | 151.409 | 1.00 | 32.53 | B | C |
| ATOM | 1817 | CB | TYR B | 176 | 45.285 | 6.690 | 151.545 | 1.00 | 35.60 | B | C |
| ATOM | 1818 | CG | TYR B | 176 | 44.636 | 7.044 | 152.867 | 1.00 | 40.89 | B | C |
| ATOM | 1819 | CD1 | TYR B | 176 | 45.334 | 6.907 | 154.070 | 1.00 | 43.33 | B | C |
| ATOM | 1820 | CE1 | TYR B | 176 | 44.736 | 7.204 | 155.290 | 1.00 | 43.67 | B | C |
| ATOM | 1821 | CD2 | TYR B | 176 | 43.318 | 7.492 | 152.922 | 1.00 | 42.09 | B | C |
| ATOM | 1822 | CE2 | TYR B | 176 | 42.711 | 7.794 | 154.140 | 1.00 | 42.63 | B | C |
| ATOM | 1823 | CZ | TYR B | 176 | 43.426 | 7.647 | 155.317 | 1.00 | 43.63 | B | C |
| ATOM | 1824 | OH | TYR B | 176 | 42.835 | 7.947 | 156.519 | 1.00 | 45.29 | B | O |
| ATOM | 1825 | C | TYR B | 176 | 46.309 | 4.931 | 150.089 | 1.00 | 30.01 | B | C |
| ATOM | 1826 | O | TYR B | 176 | 46.907 | 5.825 | 149.503 | 1.00 | 29.44 | B | O |
| ATOM | 1827 | N | PRO B | 177 | 46.255 | 3.686 | 149.604 | 1.00 | 29.94 | B | N |
| ATOM | 1828 | CD | PRO B | 177 | 45.706 | 2.475 | 150.238 | 1.00 | 28.60 | B | C |
| ATOM | 1829 | CA | PRO B | 177 | 46.918 | 3.366 | 148.335 | 1.00 | 28.82 | B | C |
| ATOM | 1830 | CB | PRO B | 177 | 46.838 | 1.843 | 148.280 | 1.00 | 29.21 | B | C |
| ATOM | 1831 | CG | PRO B | 177 | 45.590 | 1.533 | 149.067 | 1.00 | 30.96 | B | C |
| ATOM | 1832 | C | PRO B | 177 | 46.253 | 4.029 | 147.128 | 1.00 | 28.62 | B | C |
| ATOM | 1833 | O | PRO B | 177 | 45.070 | 4.372 | 147.167 | 1.00 | 30.61 | B | O |
| ATOM | 1834 | N | PHE B | 178 | 47.023 | 4.225 | 146.062 | 1.00 | 26.71 | B | N |
| ATOM | 1835 | CA | PHE B | 178 | 46.475 | 4.825 | 144.851 | 1.00 | 26.70 | B | C |
| ATOM | 1836 | CB | PHE B | 178 | 47.568 | 5.547 | 144.055 | 1.00 | 22.42 | B | C |
| ATOM | 1837 | CG | PHE B | 178 | 48.019 | 6.842 | 144.687 | 1.00 | 17.95 | B | C |
| ATOM | 1838 | CD1 | PHE B | 178 | 47.108 | 7.871 | 144.919 | 1.00 | 16.47 | B | C |
| ATOM | 1839 | CD2 | PHE B | 178 | 49.349 | 7.029 | 145.058 | 1.00 | 17.30 | B | C |
| ATOM | 1840 | CE1 | PHE B | 178 | 47.511 | 9.069 | 145.511 | 1.00 | 14.31 | B | C |
| ATOM | 1841 | CE2 | PHE B | 178 | 49.765 | 8.221 | 145.650 | 1.00 | 14.81 | B | C |
| ATOM | 1842 | CZ | PHE B | 178 | 48.842 | 9.245 | 145.877 | 1.00 | 16.31 | B | C |
| ATOM | 1843 | C | PHE B | 178 | 45.810 | 3.733 | 144.017 | 1.00 | 26.77 | B | C |
| ATOM | 1844 | O | PHE B | 178 | 46.020 | 2.546 | 144.261 | 1.00 | 23.82 | B | O |
| ATOM | 1845 | N | ASP B | 179 | 45.017 | 4.142 | 143.032 | 1.00 | 28.61 | B | N |
| ATOM | 1846 | CA | ASP B | 179 | 44.259 | 3.209 | 142.203 | 1.00 | 29.98 | B | C |
| ATOM | 1847 | CS | ASP B | 179 | 42.808 | 3.674 | 142.164 | 1.00 | 29.97 | B | C |
| ATOM | 1848 | CG | ASP B | 179 | 42.698 | 5.131 | 141.810 | 1.00 | 30.41 | B | C |
| ATOM | 1849 | OD1 | ASP B | 179 | 43.709 | 5.661 | 141.331 | 1.00 | 35.31 | B | O |
| ATOM | 1850 | OD2 | ASP B | 179 | 41.631 | 5.750 | 141.995 | 1.00 | 33.11 | B | O |
| ATOM | 1851 | C | ASP B | 179 | 44.731 | 2.976 | 140.768 | 1.00 | 30.43 | B | C |
| ATOM | 1852 | O | ASP B | 179 | 43.924 | 2.646 | 139.909 | 1.00 | 30.49 | B | O |
| ATOM | 1853 | N | GLY B | 180 | 46.020 | 3.128 | 140.499 | 1.00 | 30.89 | B | N |
| ATOM | 1854 | CA | GLY B | 180 | 46.493 | 2.905 | 139.146 | 1.00 | 29.05 | B | C |
| ATOM | 1855 | C | GLY B | 180 | 46.142 | 4.064 | 138.237 | 1.00 | 29.44 | B | C |
| ATOM | 1856 | O | GLY B | 180 | 45.789 | 5.131 | 138.723 | 1.00 | 25.58 | B | O |
| ATOM | 1857 | N | LYS B | 181 | 46.217 | 3.849 | 136.925 | 1.00 | 29.06 | B | N |
| ATOM | 1858 | CA | LYS B | 181 | 45.936 | 4.895 | 135.943 | 1.00 | 30.26 | B | C |
| ATOM | 1859 | CB | LYS B | 181 | 46.100 | 4.322 | 134.535 | 1.00 | 33.27 | B | C |
| ATOM | 1860 | CG | LYS B | 181 | 45.992 | 5.346 | 133.426 | 1.00 | 36.93 | B | C |
| ATOM | 1861 | CD | LYS B | 181 | 46.258 | 4.704 | 132.077 | 1.00 | 41.28 | B | C |
| ATOM | 1862 | CE | LYS B | 181 | 46.044 | 5.691 | 130.936 | 1.00 | 44.88 | B | C |
| ATOM | 1863 | NZ | LYS B | 181 | 44.634 | 6.1761 | 30.859 | 1.00 | 43.95 | B | N |
| ATOM | 1864 | C | LYS B | 181 | 44.565 | 5.565 | 136.087 | 1.00 | 28.94 | B | C |
| ATOM | 1865 | O | LYS B | 181 | 43.545 | 4.901 | 136.239 | 1.00 | 30.47 | B | O |
| ATOM | 1866 | N | ASP B | 182 | 44.561 | 6.892 | 136.034 | 1.00 | 29.99 | B | N |
| ATOM | 1867 | CA | ASP B | 182 | 43.347 | 7.692 | 136.166 | 1.00 | 30.47 | B | C |
| ATOM | 1868 | CB | ASP B | 182 | 42.367 | 7.376 | 135.035 | 1.00 | 35.39 | B | C |
| ATOM | 1869 | CG | ASP B | 182 | 42.877 | 7.826 | 133.684 | 1.00 | 38.40 | B | C |
| ATOM | 1870 | OD1 | ASP B | 182 | 43.081 | 9.045 | 133.491 | 1.00 | 39.28 | B | O |
| ATOM | 1871 | OD2 | ASP B | 182 | 43.078 | 6.954 | 132.814 | 1.00 | 42.49 | B | O |
| ATOM | 1872 | C | ASP B | 182 | 42.642 | 7.509 | 137.506 | 1.00 | 31.65 | B | C |
| ATOM | 1873 | O | ASP B | 182 | 43.099 | 6.765 | 138.363 | 1.00 | 32.44 | B | O |
| ATOM | 1874 | N | GLY B | 183 | 41.509 | 8.181 | 137.670 | 1.00 | 29.41 | B | N |
| ATOM | 1875 | CA | GLY B | 183 | 40.774 | 8.091 | 138.912 | 1.00 | 27.84 | B | C |
| ATOM | 1876 | C | GLY B | 183 | 41.335 | 9.123 | 139.864 | 1.00 | 27.88 | B | C |
| ATOM | 1877 | O | GLY B | 183 | 41.355 | 10.310 | 139.549 | 1.00 | 29.24 | B | O |
| ATOM | 1878 | N | LEU B | 184 | 41.792 | 8.678 | 141.030 | 1.00 | 27.15 | B | N |
| ATOM | 1879 | CA | LEU B | 184 | 42.376 | 9.585 | 142.015 | 1.00 | 25.82 | B | C |
| ATOM | 1880 | CB | LEU B | 184 | 42.493 | 8.878 | 143.359 | 1.00 | 27.95 | B | C |
| ATOM | 1881 | CG | LEU B | 184 | 42.940 | 9.772 | 144.507 | 1.00 | 30.41 | B | C |
| ATOM | 1882 | CD1 | LEU B | 184 | 41.855 | 10.797 | 144.779 | 1.00 | 28.19 | B | C |
| ATOM | 1883 | CD2 | LEU B | 184 | 43.205 | 8.927 | 145.746 | 1.00 | 34.06 | B | C |
| ATOM | 1884 | C | LEU B | 184 | 43.771 | 9.971 | 141.520 | 1.00 | 22.60 | B | C |
| ATOM | 1885 | O | LEU B | 184 | 44.593 | 9.106 | 141.265 | 1.00 | 17.85 | B | O |
| ATOM | 1886 | N | LEU B | 185 | 44.047 | 11.262 | 141.397 | 1.00 | 22.16 | B | N |
| ATOM | 1887 | CA | LEU B | 185 | 45.346 | 11.700 | 140.884 | 1.00 | 22.33 | B | C |
| ATOM | 1888 | CB | LEU B | 185 | 45.177 | 13.002 | 140.095 | 1.00 | 19.27 | B | C |
| ATOM | 1889 | CG | LEU B | 185 | 44.127 | 12.970 | 138.979 | 1.00 | 20.81 | B | C |
| ATOM | 1890 | CD1 | LEU B | 185 | 44.110 | 14.305 | 138.252 | 1.00 | 12.81 | B | C |
| ATOM | 1891 | CD2 | LEU B | 185 | 44.425 | 11.823 | 138.013 | 1.00 | 17.71 | B | C |
| ATOM | 1892 | C | LEU B | 185 | 46.400 | 11.897 | 141.965 | 1.00 | 23.14 | B | C |
| ATOM | 1893 | O | LEU B | 185 | 47.593 | 11.658 | 141.749 | 1.00 | 22.98 | B | O |
| ATOM | 1894 | N | ALA B | 186 | 45.948 | 12.331 | 143.131 | 1.00 | 23.03 | B | N |
| ATOM | 1895 | CA | ALA B | 186 | 46.830 | 12.585 | 144.255 | 1.00 | 22.63 | B | C |
| ATOM | 1896 | CB | ALA B | 186 | 47.717 | 13.768 | 143.942 | 1.00 | 21.82 | B | C |
| ATOM | 1897 | C | ALA B | 186 | 45.974 | 12.896 | 145.472 | 1.00 | 23.10 | B | C |
| ATOM | 1898 | O | ALA B | 186 | 44.751 | 12.932 | 145.378 | 1.00 | 25.16 | B | O |
| ATOM | 1899 | N | HIS B | 187 | 46.622 | 13.104 | 146.613 | 1.00 | 22.01 | B | N |
| ATOM | 1900 | CA | HIS B | 187 | 45.927 | 13.458 | 147.840 | 1.00 | 21.61 | B | C |
| ATOM | 1901 | CB | HIS B | 187 | 45.218 | 12.237 | 148.451 | 1.00 | 19.44 | B | C |
| ATOM | 1902 | CG | HIS B | 187 | 46.126 | 11.110 | 148.838 | 1.00 | 21.62 | B | C |
| ATOM | 1903 | CD2 | HIS B | 187 | 47.343 | 11.101 | 149.434 | 1.00 | 23.01 | B | C |
| ATOM | 1904 | ND1 | HIS B | 187 | 45.771 | 9.787 | 148.673 | 1.00 | 20.29 | B | N |
| ATOM | 1905 | CE1 | HIS B | 187 | 46.730 | 9.013 | 149.151 | 1.00 | 20.79 | B | C |
| ATOM | 1906 | NE2 | HIS B | 187 | 47.695 | 9.786 | 149.619 | 1.00 | 19.38 | B | N |
| ATOM | 1907 | C | HIS B | 187 | 46.898 | 14.097 | 148.822 | 1.00 | 22.17 | B | C |
| ATOM | 1908 | O | HIS B | 187 | 48.086 | 13.786 | 148.818 | 1.00 | 21.16 | B | O |
| ATOM | 1909 | N | ALA B | 188 | 46.386 | 15.015 | 149.637 | 1.00 | 19.90 | B | N |
| ATOM | 1910 | CA | ALA B | 188 | 47.191 | 15.727 | 150.615 | 1.00 | 21.04 | B | C |
| ATOM | 1911 | CB | ALA B | 188 | 47.386 | 17.165 | 150.169 | 1.00 | 16.20 | B | C |
| ATOM | 1912 | C | ALA B | 188 | 46.511 | 15.689 | 151.981 | 1.00 | 24.29 | B | C |
| ATOM | 1913 | O | ALA B | 188 | 45.342 | 15.313 | 152.091 | 1.00 | 23.00 | B | O |
| ATOM | 1914 | N | PHE B | 189 | 47.248 | 16.076 | 153.020 | 1.00 | 25.22 | B | N |
| ATOM | 1915 | CA | PHE B | 189 | 46.712 | 16.081 | 154.373 | 1.00 | 23.69 | B | C |
| ATOM | 1916 | CB | PHE B | 189 | 47.532 | 15.162 | 155.290 | 1.00 | 23.93 | B | C |
| ATOM | 1917 | CG | PHE B | 189 | 47.565 | 13.731 | 154.839 | 1.00 | 26.85 | B | C |
| ATOM | 1918 | CD1 | PHE B | 189 | 48.483 | 13.308 | 153.881 | 1.00 | 29.60 | B | C |
| ATOM | 1919 | CD2 | PHE B | 189 | 46.668 | 12.806 | 155.358 | 1.00 | 27.63 | B | C |
| ATOM | 1920 | CE1 | PHE B | 189 | 48.510 | 11.984 | 153.447 | 1.00 | 27.95 | B | C |
| ATOM | 1921 | CE2 | PHE B | 189 | 46.685 | 11.480 | 154.930 | 1.00 | 29.13 | B | C |
| ATOM | 1922 | CZ | PHE B | 189 | 47.609 | 11.069 | 153.972 | 1.00 | 29.53 | B | C |
| ATOM | 1923 | C | PHE B | 189 | 46.712 | 17.491 | 154.932 | 1.00 | 24.15 | B | C |
| ATOM | 1924 | O | PHE B | 189 | 47.600 | 18.286 | 154.632 | 1.00 | 23.64 | B | O |
| ATOM | 1925 | N | PRO B | 190 | 45.703 | 17.818 | 155.761 | 1.00 | 25.45 | B | N |
| ATOM | 1926 | CD | PRO B | 190 | 44.626 | 16.901 | 156.165 | 1.00 | 24.82 | B | C |
| ATOM | 1927 | CA | PRO B | 190 | 45.535 | 19.135 | 156.399 | 1.00 | 23.84 | B | C |
| ATOM | 1928 | CB | PRO B | 190 | 44.211 | 18.998 | 157.151 | 1.00 | 23.85 | B | C |
| ATOM | 1929 | CG | PRO B | 190 | 43.517 | 17.859 | 156.470 | 1.00 | 27.72 | B | C |
| ATOM | 1930 | C | PRO B | 190 | 46.693 | 19.452 | 157.355 | 1.00 | 24.12 | B | C |
| ATOM | 1931 | O | PRO B | 190 | 47.364 | 18.544 | 157.851 | 1.00 | 24.59 | B | O |
| ATOM | 1932 | N | PRO B | 191 | 46.929 | 20.745 | 157.633 | 1.00 | 23.67 | B | N |
| ATOM | 1933 | CD | PRO B | 191 | 46.130 | 21.889 | 157.160 | 1.00 | 22.01 | B | C |
| ATOM | 1934 | CA | PRO B | 191 | 48.005 | 21.193 | 158.527 | 1.00 | 24.52 | B | C |
| ATOM | 1935 | CB | PRO B | 191 | 47.668 | 22.660 | 158.756 | 1.00 | 23.21 | B | C |
| ATOM | 1936 | CG | PRO B | 191 | 47.036 | 23.056 | 157.463 | 1.00 | 23.95 | B | C |
| ATOM | 1937 | C | PRO B | 191 | 48.077 | 20.402 | 159.834 | 1.00 | 26.78 | B | C |
| ATOM | 1938 | O | PRO B | 191 | 47.054 | 19.982 | 160.374 | 1.00 | 27.80 | B | O |
| ATOM | 1939 | N | GLY B | 192 | 49.294 | 20.207 | 160.334 | 1.00 | 26.68 | B | N |
| ATOM | 1940 | CA | GLY B | 192 | 49.490 | 19.459 | 161.563 | 1.00 | 25.28 | B | C |
| ATOM | 1941 | C | GLY B | 192 | 50.851 | 18.786 | 161.573 | 1.00 | 26.25 | B | C |
| ATOM | 1942 | O | GLY B | 192 | 51.653 | 19.026 | 160.671 | 1.00 | 27.65 | B | O |
| ATOM | 1943 | N | PRO B | 193 | 51.145 | 17.935 | 162.574 | 1.00 | 26.70 | B | N |
| ATOM | 1944 | CD | PRO B | 193 | 50.356 | 17.760 | 163.808 | 1.00 | 23.89 | B C | |
| ATOM | 1945 | CA | PRO B | 193 | 52.428 | 17.233 | 162.685 | 1.00 | 26.43 | B | C |
| ATOM | 1946 | CB | PRO B | 193 | 52.572 | 17.049 | 164.189 | 1.00 | 26.13 | B | C |
| ATOM | 1947 | CG | PRO B | 193 | 51.173 | 16.742 | 164.588 | 1.00 | 24.53 | B | C |
| ATOM | 1948 | C | PRO B | 193 | 52.463 | 15.901 | 161.947 | 1.00 | 25.56 | B | C |
| ATOM | 1949 | O | PRO B | 193 | 51.423 | 15.356 | 161.592 | 1.00 | 28.95 | B | O |
| ATOM | 1950 | N | GLY B | 194 | 53.665 | 15.379 | 161.721 | 1.00 | 24.12 | B | N |
| ATOM | 1951 | CA | GLY B | 194 | 53.803 | 14.100 | 161.046 | 1.00 | 22.38 | B | C |
| ATOM | 1952 | C | GLY B | 194 | 53.338 | 14.098 | 159.605 | 1.00 | 23.14 | B | C |
| ATOM | 1953 | O | GLY B | 194 | 53.602 | 15.036 | 158.868 | 1.00 | 21.24 | B | O |
| ATOM | 1954 | N | ILE B | 195 | 52.633 | 13.048 | 159.202 | 1.00 | 24.37 | B | N |
| ATOM | 1955 | CA | ILE B | 195 | 52.147 | 12.941 | 157.832 | 1.00 | 26.26 | B | C |
| ATOM | 1956 | CB | ILE B | 195 | 51.440 | 11.590 | 157.613 | 1.00 | 27.60 | B | C |
| ATOM | 1957 | CG2 | ILE B | 195 | 50.131 | 11.561 | 158.386 | 1.00 | 27.08 | B | C |
| ATOM | 1958 | CG1 | ILE B | 195 | 51.200 | 11.365 | 156.119 | 1.00 | 29.76 | B | C |
| ATOM | 1959 | CD1 | ILE B | 195 | 50.620 | 10.012 | 155.789 | 1.00 | 31.30 | B | C |
| ATOM | 1960 | C | ILE B | 195 | 51.194 | 14.085 | 157.479 | 1.00 | 26.36 | B | C |
| ATOM | 1961 | O | ILE B | 195 | 50.921 | 14.343 | 156.309 | 1.00 | 27.06 | B | O |
| ATOM | 1962 | N | GLN B | 196 | 50.704 | 14.781 | 158.498 | 1.00 | 26.62 | B | N |
| ATOM | 1963 | CA | GLN B | 196 | 49.790 | 15.901 | 158.298 | 1.00 | 26.46 | B | C |
| ATOM | 1964 | CB | GLN B | 196 | 49.359 | 16.457 | 159.654 | 1.00 | 29.46 | B | C |
| ATOM | 1965 | CG | GLN B | 196 | 47.864 | 16.383 | 159.914 | 1.00 | 38.14 | B | C |
| ATOM | 1966 | CD | GLN B | 196 | 47.323 | 14.967 | 159.860 | 1.00 | 41.94 | B | C |
| ATOM | 1967 | OE1 | GLN B | 196 | 46.116 | 14.751 | 159.940 | 1.00 | 44.27 | B | O |
| ATOM | 1968 | NE2 | GLN B | 196 | 48.215 | 13.993 | 159.727 | 1.00 | 46.11 | B | N |
| ATOM | 1969 | C | GLN B | 196 | 50.459 | 16.998 | 157.482 | 1.00 | 23.79 | B | C |
| ATOM | 1970 | O | GLN B | 196 | 51.576 | 17.367 | 157.768 | 1.00 | 25.59 | B | O |
| ATOM | 1971 | N | GLY B | 197 | 49.774 | 17.524 | 156.472 | 1.00 | 23.38 | B | N |
| ATOM | 1972 | CA | GLY B | 197 | 50.364 | 18.568 | 155.650 | 1.00 | 18.35 | B | C |
| ATOM | 1973 | C | GLY B | 197 | 51.149 | 18.047 | 154.450 | 1.00 | 20.05 | B | C |
| ATOM | 1974 | O | GLY B | 197 | 51.606 | 18.835 | 153.619 | 1.00 | 21.28 | B | O |
| ATOM | 1975 | N | ASP B | 198 | 51.308 | 16.727 | 154.354 | 1.00 | 18.69 | B | N |
| ATOM | 1976 | CA | ASP B | 198 | 52.049 | 16.115 | 153.253 | 1.00 | 18.67 | B | C |
| ATOM | 1977 | CB | ASP B | 198 | 52.557 | 14.730 | 153.668 | 1.00 | 17.24 | B | C |
| ATOM | 1978 | CG | ASP B | 198 | 53.613 | 14.794 | 154.764 | 1.00 | 19.44 | B | C |
| ATOM | 1979 | OD1 | ASP B | 198 | 53.978 | 15.912 | 155.160 | 1.00 | 14.37 | B | O |
| ATOM | 1980 | OD2 | ASP B | 198 | 54.080 | 13.730 | 155.220 | 1.00 | 16.31 | B | O |
| ATOM | 1981 | C | ASP B | 198 | 51.203 | 15.996 | 151.974 | 1.00 | 20.96 | B | C |
| ATOM | 1982 | O | ASP B | 198 | 49.971 | 16.042 | 152.029 | 1.00 | 21.22 | B | O |
| ATOM | 1983 | N | ALA B | 199 | 51.872 | 15.836 | 150.831 | 1.00 | 18.26 | B | N |
| ATOM | 1984 | CA | ALA B | 199 | 51.197 | 15.722 | 149.536 | 1.00 | 17.33 | B | C |
| ATOM | 1985 | CB | ALA B | 199 | 51.326 | 17.027 | 148.771 | 1.00 | 16.82 | B | C |
| ATOM | 1986 | C | ALA B | 199 | 51.803 | 14.586 | 148.728 | 1.00 | 18.72 | B | C |
| ATOM | 1987 | O | ALA B | 199 | 53.004 | 14.569 | 148.483 | 1.00 | 20.07 | B | O |
| ATOM | 1988 | N | HIS B | 200 | 50.968 | 13.639 | 148.314 | 1.00 | 17.89 | B | N |
| ATOM | 1989 | CA | HIS B | 200 | 51.424 | 12.481 | 147.555 | 1.00 | 15.98 | B | C |
| ATOM | 1990 | CB | HIS B | 200 | 51.092 | 11.215 | 148.334 | 1.00 | 17.39 | B | C |
| ATOM | 1991 | CG | HIS B | 200 | 51.623 | 11.212 | 149.736 | 1.00 | 20.36 | B | C |
| ATOM | 1992 | CD2 | HIS B | 200 | 52.599 | 11.944 | 150.325 | 1.00 | 17.40 | B | C |
| ATOM | 1993 | ND1 | HIS B | 200 | 51.160 | 10.349 | 150.704 | 1.00 | 18.51 | B | N |
| ATOM | 1994 | CE1 | HIS B | 200 | 51.827 | 10.546 | 151.826 | 1.00 | 17.19 | B | C |
| ATOM | 1995 | NB2 | HIS B | 200 | 52.706 | 11.508 | 151.623 | 1.00 | 17.14 | B | N |
| ATOM | 1996 | C | HIS B | 200 | 50.786 | 12.427 | 146.168 | 1.00 | 18.64 | B | C |
| ATOM | 1997 | O | HIS B | 200 | 49.591 | 12.668 | 146.016 | 1.00 | 18.44 | B | O |
| ATOM | 1998 | N | PHE B | 201 | 51.594 | 12.105 | 145.160 | 1.00 | 19.00 | B | N |
| ATOM | 1999 | CA | PHE B | 201 | 51.128 | 12.046 | 143.776 | 1.00 | 15.37 | B | C |
| ATOM | 2000 | CB | PHE B | 201 | 51.929 | 13.027 | 142.912 | 1.00 | 16.11 | B | C |
| ATOM | 2001 | CG | PHE B | 201 | 51.907 | 14.444 | 143.421 | 1.00 | 14.27 | B | C |
| ATOM | 2002 | CD1 | PHE B | 201 | 52.747 | 14.840 | 144.459 | 1.00 | 15.41 | B | C |
| ATOM | 2003 | CD2 | PHE B | 201 | 51.017 | 15.374 | 142.887 | 1.00 | 13.45 | B | C |
| ATOM | 2004 | CE1 | PHE B | 201 | 52.704 | 16.143 | 144.961 | 1.00 | 15.16 | B | C |
| ATOM | 2005 | CE2 | PHE B | 201 | 50.962 | 16.685 | 143.380 | 1.00 | 14.52 | B | C |
| ATOM | 2006 | CZ | PHE B | 201 | 51.806 | 17.071 | 144.418 | 1.00 | 14.98 | B | C |
| ATOM | 2007 | C | PHE B | 201 | 51.227 | 10.650 | 143.185 | 1.00 | 15.36 | B | C |
| ATOM | 2008 | O | PHE B | 201 | 52.257 | 9.993 | 143.293 | 1.00 | 15.05 | B | O |
| ATOM | 2009 | N | ASP B | 202 | 50.144 | 10.204 | 142.555 | 1.00 | 16.99 | B | N |
| ATOM | 2010 | CA | ASP B | 202 | 50.094 | 8.877 | 141.941 | 1.00 | 18.47 | B | C |
| ATOM | 2011 | CB | ASP B | 202 | 48.660 | 8.502 | 141.583 | 1.00 | 16.97 | B | C |
| ATOM | 2012 | CG | ASP B | 202 | 48.546 | 7.080 | 141.116 | 1.00 | 17.69 | B | C |
| ATOM | 2013 | OD1 | ASP B | 202 | 49.567 | 6.496 | 140.725 | 1.00 | 24.22 | B | O |
| ATOM | 2014 | OD2 | ASP B | 202 | 47.442 | 6.530 | 141.132 | 1.00 | 22.67 | B | O |
| ATOM | 2015 | C | ASP B | 202 | 50.944 | 8.843 | 140.673 | 1.00 | 17.99 | B | C |
| ATOM | 2016 | O | ASP B | 202 | 50.604 | 9.460 | 139.670 | 1.00 | 16.40 | B | O |
| ATOM | 2017 | N | ASP B | 203 | 52.039 | 8.101 | 140.722 | 1.00 | 18.18 | B | N |
| ATOM | 2018 | CA | ASP B | 203 | 52.943 | 8.020 | 139.593 | 1.00 | 19.10 | B | C |
| ATOM | 2019 | CB | ASP B | 203 | 54.360 | 7.745 | 140.098 | 1.00 | 18.63 | B | C |
| ATOM | 2020 | CG | ASP B | 203 | 55.424 | 8.234 | 139.147 | 1.00 | 21.55 | B | C |
| ATOM | 2021 | OD1 | ASP B | 203 | 55.153 | 9.165 | 138.359 | 1.00 | 22.68 | B | O |
| ATOM | 2022 | OD2 | ASP B | 203 | 56.546 | 7.700 | 139.204 | 1.00 | 24.38 | B | O |
| ATOM | 2023 | C | ASP B | 203 | 52.522 | 6.977 | 138.560 | 1.00 | 22.02 | B | C |
| ATOM | 2024 | O | ASP B | 203 | 53.328 | 6.553 | 137.739 | 1.00 | 22.79 | B | O |
| ATOM | 2025 | N | ASP B | 204 | 51.274 | 6.529 | 138.627 | 1.00 | 21.73 | B | N |
| ATOM | 2026 | CA | ASP B | 204 | 50.772 | 5.589 | 137.631 | 1.00 | 20.62 | B | C |
| ATOM | 2027 | CB | ASP B | 204 | 49.729 | 4.635 | 138.212 | 1.00 | 19.80 | B | C |
| ATOM | 2028 | CG | ASP B | 204 | 50.304 | 3.270 | 138.551 | 1.00 | 22.45 | B | C |
| ATOM | 2029 | OD1 | ASP B | 204 | 51.334 | 2.884 | 137.963 | 1.00 | 20.45 | B | O |
| ATOM | 2030 | OD2 | ASP B | 204 | 49.709 | 2.571 | 139.399 | 1.00 | 23.05 | B | O |
| ATOM | 2031 | C | ASP B | 204 | 50.123 | 6.478 | 136.570 | 1.00 | 20.79 | B | C |
| ATOM | 2032 | O | ASP B | 204 | 49.739 | 6.016 | 135.504 | 1.00 | 23.31 | B | O |
| ATOM | 2033 | N | GLU B | 205 | 50.008 | 7.763 | 136.890 | 1.00 | 18.69 | B | N |
| ATOM | 2034 | CA | GLU B | 205 | 49.441 | 8.755 | 135.987 | 1.00 | 20.13 | B | C |
| ATOM | 2035 | CB | GLU B | 205 | 48.874 | 9.931 | 136.778 | 1.00 | 20.83 | B | C |
| ATOM | 2036 | CG | GLU B | 205 | 47.935 | 9.526 | 137.894 | 1.00 | 29.96 | B | C |
| ATOM | 2037 | CD | GLU B | 205 | 46.627 | 8.986 | 137.377 | 1.00 | 33.57 | B | C |
| ATOM | 2038 | OB1 | GLU B | 205 | 46.617 | 8.360 | 136.293 | 1.00 | 36.59 | B | O |
| ATOM | 2039 | OE2 | GLU B | 205 | 45.607 | 9.185 | 138.060 | 1.00 | 40.04 | B | O |
| ATOM | 2040 | C | GLU B | 205 | 50.560 | 9.285 | 135.099 | 1.00 | 21.01 | B | C |
| ATOM | 2041 | O | GLU B | 205 | 51.725 | 9.238 | 135.477 | 1.00 | 19.13 | B | O |
| ATOM | 2042 | N | LEU B | 206 | 50.207 | 9.795 | 133.924 | 1.00 | 18.61 | B | N |
| ATOM | 2043 | CA | LEU B | 206 | 51.204 | 10.366 | 133.026 | 1.00 | 17.96 | B | C |
| ATOM | 2044 | CB | LEU B | 206 | 50.758 | 10.242 | 131.568 | 1.00 | 18.74 | B | C |
| ATOM | 2045 | CG | LEU B | 206 | 51.617 | 10.985 | 130.532 | 1.00 | 19.98 | B | C |
| ATOM | 2046 | CD1 | LEU B | 206 | 53.062 | 10.519 | 130.627 | 1.00 | 18.61 | B | C |
| ATOM | 2047 | CD2 | LEU B | 206 | 51.064 | 10.740 | 129.131 | 1.00 | 17.02 | B | C |
| ATOM | 2048 | C | LEU B | 206 | 51.312 | 11.837 | 133.399 | 1.00 | 18.42 | B | C |
| ATOM | 2049 | O | LEU B | 206 | 50.379 | 12.603 | 133.160 | 1.00 | 18.49 | B | O |
| ATOM | 2050 | N | TRP B | 207 | 52.434 | 12.230 | 133.998 | 1.00 | 17.94 | B | N |
| ATOM | 2051 | CA | TRP B | 207 | 52.618 | 13.622 | 134.401 | 1.00 | 16.68 | B | C |
| ATOM | 2052 | CB | TRP B | 207 | 53.454 | 13.723 | 135.694 | 1.00 | 15.77 | B | C |
| ATOM | 2053 | CG | TRP B | 207 | 52.752 | 13.143 | 136.870 | 1.00 | 14.61 | B | C |
| ATOM | 2054 | CD2 | TRP B | 207 | 51.546 | 13.636 | 137.469 | 1.00 | 14.73 | B | C |
| ATOM | 2055 | CE2 | TRP B | 207 | 51.165 | 12.708 | 138.464 | 1.00 | 14.79 | B | C |
| ATOM | 2056 | CE3 | TRP B | 207 | 50.744 | 14.769 | 137.255 | 1.00 | 16.70 | B | C |
| ATOM | 2057 | CD1 | TRP B | 207 | 53.051 | 11.979 | 137.511 | 1.00 | 13.93 | B | C |
| ATOM | 2058 | NE1 | TRP B | 207 | 52.101 | 11.708 | 138.467 | 1.00 | 15.76 | B | N |
| ATOM | 2059 | CZ2 | TRP B | 207 | 50.012 | 12.876 | 139.248 | 1.00 | 13.38 | B | C |
| ATOM | 2060 | CZ3 | TRP B | 207 | 49.591 | 14.935 | 138.034 | 1.00 | 13.30 | B | C |
| ATOM | 2061 | CH2 | TRP B | 207 | 49.241 | 13.990 | 139.016 | 1.00 | 13.83 | B | C |
| ATOM | 2062 | C | TRP B | 207 | 53.267 | 14.435 | 133.299 | 1.00 | 16.25 | B | C |
| ATOM | 2063 | O | TRP B | 207 | 54.230 | 14.003 | 132.665 | 1.00 | 14.91 | B | O |
| ATOM | 2064 | N | SER B | 208 | 52.731 | 15.630 | 133.092 | 1.00 | 16.82 | B | N |
| ATOM | 2065 | CA | SER B | 208 | 53.218 | 16.521 | 132.061 | 1.00 | 15.97 | B | C |
| ATOM | 2066 | CB | SER B | 208 | 52.497 | 16.187 | 130.747 | 1.00 | 16.80 | B | C |
| ATOM | 2067 | OG | SER B | 208 | 53.111 | 16.799 | 129.634 | 1.00 | 12.64 | B | O |
| ATOM | 2068 | C | SER B | 208 | 52.924 | 17.953 | 132.496 | 1.00 | 17.15 | B | C |
| ATOM | 2069 | O | SER B | 208 | 52.677 | 18.207 | 133.676 | 1.00 | 20.26 | B | O |
| ATOM | 2070 | N | LEU B | 209 | 52.954 | 18.881 | 131.543 | 1.00 | 15.88 | B | N |
| ATOM | 2071 | CA | LEU B | 209 | 52.680 | 20.294 | 131.800 | 1.00 | 15.39 | B | C |
| ATOM | 2072 | CB | LEU B | 209 | 53.972 | 21.049 | 132.105 | 1.00 | 12.60 | B | C |
| ATOM | 2073 | CG | LEU B | 209 | 54.744 | 20.758 | 133.401 | 1.00 | 16.29 | B | C |
| ATOM | 2074 | CD1 | LEU B | 209 | 56.029 | 21.560 | 133.386 | 1.00 | 12.54 | B | C |
| ATOM | 2075 | CD2 | LEU B | 209 | 53.904 | 21.118 | 134.638 | 1.00 | 12.78 | B | C |
| ATOM | 2076 | C | LEU B | 209 | 52.044 | 20.901 | 130.558 | 1.00 | 16.86 | B | C |
| ATOM | 2077 | O | LEU B | 209 | 52.335 | 20.471 | 129.453 | 1.00 | 16.65 | B | O |
| ATOM | 2078 | N | GLY B | 210 | 51.181 | 21.898 | 130.739 | 1.00 | 20.56 | B | N |
| ATOM | 2079 | CA | GLY B | 210 | 50.545 | 22.557 | 129.602 | 1.00 | 20.23 | B | C |
| ATOM | 2080 | C | GLY B | 210 | 49.475 | 21.767 | 128.864 | 1.00 | 21.25 | B | C |
| ATOM | 2081 | O | GLY B | 210 | 48.885 | 20.835 | 129.409 | 1.00 | 21.72 | B | O |
| ATOM | 2082 | N | LYS B | 211 | 49.207 | 22.158 | 127.621 | 1.00 | 20.99 | B | N |
| ATOM | 2083 | CA | LYS B | 211 | 48.217 | 21.473 | 126.795 | 1.00 | 21.58 | B | C |
| ATOM | 2084 | CB | LYS B | 211 | 47.879 | 22.314 | 125.560 | 1.00 | 22.10 | B | C |
| ATOM | 2085 | CG | LYS B | 211 | 47.094 | 23.564 | 125.878 | 1.00 | 24.12 | B | C |
| ATOM | 2086 | CD | LYS B | 211 | 46.828 | 24.410 | 124.647 | 1.00 | 27.19 | B | C |
| ATOM | 2087 | CE | LYS B | 211 | 48.099 | 25.058 | 124.149 | 1.00 | 30.98 | B | C |
| ATOM | 2088 | NZ | LYS B | 211 | 47.812 | 26.127 | 123.147 | 1.00 | 36.83 | B | N |
| ATOM | 2089 | C | LYS B | 211 | 48.804 | 20.142 | 126.355 | 1.00 | 22.13 | B | C |
| ATOM | 2090 | O | LYS B | 211 | 50.029 | 19.997 | 126.286 | 1.00 | 23.20 | B | O |
| ATOM | 2091 | N | GLY B | 212 | 47.940 | 19.180 | 126.044 | 1.00 | 18.79 | B | N |
| ATOM | 2092 | CA | GLY B | 212 | 48.420 | 17.877 | 125.623 | 1.00 | 17.78 | B | C |
| ATOM | 2093 | C | GLY B | 212 | 47.925 | 16.758 | 126.521 | 1.00 | 18.87 | B | C |
| ATOM | 2094 | O | GLY B | 212 | 47.238 | 17.010 | 127.515 | 1.00 | 19.58 | B | O |
| ATOM | 2095 | N | GLN B | 391 | 48.268 | 15.519 | 126.181 | 1.00 | 19.71 | B | N |
| ATOM | 2096 | CA | GLN B | 391 | 47.828 | 14.384 | 126.978 | 1.00 | 24.27 | B | C |
| ATOM | 2097 | CB | GLN B | 391 | 48.024 | 13.073 | 126.200 | 1.00 | 26.88 | B | C |
| ATOM | 2098 | CG | GLN B | 391 | 49.371 | 12.436 | 126.354 | 1.00 | 36.19 | B | C |
| ATOM | 2099 | CD | GLN B | 391 | 50.489 | 13.421 | 126.157 | 1.00 | 42.59 | B | C |
| ATOM | 2100 | OE1 | GLN B | 391 | 50.568 | 14.090 | 125.131 | 1.00 | 51.17 | B | O |
| ATOM | 2101 | NE2 | GLN B | 391 | 51.368 | 13.520 | 127.144 | 1.00 | 50.82 | B | N |
| ATOM | 2102 | C | GLN B | 391 | 48.582 | 14.369 | 128.311 | 1.00 | 22.67 | B | C |
| ATOM | 2103 | O | GLN B | 391 | 49.614 | 15.024 | 128.456 | 1.00 | 19.97 | B | O |
| ATOM | 2104 | N | GLY B | 392 | 48.050 | 13.635 | 129.284 | 1.00 | 22.31 | B | N |
| ATOM | 2105 | CA | GLY B | 392 | 48.672 | 13.586 | 130.592 | 1.00 | 20.72 | B | C |
| ATOM | 2106 | C | GLY B | 392 | 48.050 | 14.651 | 131.473 | 1.00 | 19.93 | B | C |
| ATOM | 2107 | O | GLY B | 392 | 47.238 | 15.452 | 131.010 | 1.00 | 20.51 | B | O |
| ATOM | 2108 | N | TYR B | 393 | 48.433 | 14.672 | 132.744 | 1.00 | 20.46 | B | N |
| ATOM | 2109 | CA | TYR B | 393 | 47.896 | 15.643 | 133.692 | 1.00 | 18.29 | B | C |
| ATOM | 2110 | CB | TYR B | 393 | 47.421 | 14.924 | 134.962 | 1.00 | 20.08 | B | C |
| ATOM | 2111 | CG | TYR B | 393 | 46.248 | 13.989 | 134.746 | 1.00 | 16.50 | B | C |
| ATOM | 2112 | CD1 | TYR B | 393 | 44.957 | 14.490 | 134.602 | 1.00 | 17.97 | B | C |
| ATOM | 2113 | CE1 | TYR B | 393 | 43.871 | 13.647 | 134.397 | 1.00 | 16.85 | B | C |
| ATOM | 2114 | CD2 | TYR B | 393 | 46.429 | 12.610 | 134.675 | 1.00 | 18.06 | B | C |
| ATOM | 2115 | CE2 | TYR B | 393 | 45.342 | 11.747 | 134.467 | 1.00 | 17.10 | B | C |
| ATOM | 2116 | CZ | TYR B | 393 | 44.064 | 12.281 | 134.329 | 1.00 | 18.56 | B | C |
| ATOM | 2117 | OH | TYR B | 393 | 42.974 | 11.463 | 134.100 | 1.00 | 18.94 | B | O |
| ATOM | 2118 | C | TYR B | 393 | 48.964 | 16.653 | 134.050 | 1.00 | 17.76 | B | C |
| ATOM | 2119 | O | TYR B | 393 | 50.138 | 16.304 | 134.130 | 1.00 | 22.05 | B | O |
| ATOM | 2120 | N | SER B | 394 | 48.560 | 17.901 | 134.271 | 1.00 | 17.69 | B | N |
| ATOM | 2121 | CA | SER B | 394 | 49.505 | 18.955 | 134.627 | 1.00 | 18.43 | B | C |
| ATOM | 2122 | CB | SER B | 394 | 48.864 | 20.331 | 134.464 | 1.00 | 15.87 | B | C |
| ATOM | 2123 | OG | SER B | 394 | 49.799 | 21.357 | 134.756 | 1.00 | 17.63 | B | O |
| ATOM | 2124 | C | SER B | 394 | 49.974 | 18.806 | 136.066 | 1.00 | 19.32 | B | C |
| ATOM | 2125 | O | SER B | 394 | 49.185 | 18.953 | 136.997 | 1.00 | 23.73 | B | O |
| ATOM | 2126 | N | LEU B | 395 | 51.253 | 18.512 | 136.255 | 1.00 | 18.56 | B | N |
| ATOM | 2127 | CA | LEU B | 395 | 51.785 | 18.373 | 137.604 | 1.00 | 17.72 | B | C |
| ATOM | 2128 | CB | LEU B | 395 | 53.265 | 17.972 | 137.556 | 1.00 | 16.48 | B | C |
| ATOM | 2129 | CG | LEU B | 395 | 53.993 | 17.751 | 138.889 | 1.00 | 15.25 | B | C |
| ATOM | 2130 | CD1 | LEU B | 395 | 53.311 | 16.631 | 139.670 | 1.00 | 13.74 | B | C |
| ATOM | 2131 | CD2 | LEU B | 395 | 55.457 | 17.409 | 138.628 | 1.00 | 10.40 | B | C |
| ATOM | 2132 | C | LEU B | 395 | 51.624 | 19.710 | 138.329 | 1.00 | 18.62 | B | C |
| ATOM | 2133 | O | LEU B | 395 | 51.285 | 19.746 | 139.507 | 1.00 | 19.25 | B | O |
| ATOM | 2134 | N | PHE B | 396 | 51.853 | 20.803 | 137.605 | 1.00 | 16.65 | B | N |
| ATOM | 2135 | CA | PHE B | 396 | 51.741 | 22.152 | 138.152 | 1.00 | 17.42 | B | C |
| ATOM | 2136 | CB | PHE B | 396 | 52.035 | 23.181 | 137.057 | 1.00 | 16.61 | B | C |
| ATOM | 2137 | CG | PHE B | 396 | 51.661 | 24.592 | 137.421 | 1.00 | 17.86 | B | C |
| ATOM | 2138 | CD1 | PHE B | 396 | 52.306 | 25.261 | 138.456 | 1.00 | 17.66 | B | C |
| ATOM | 2139 | CD2 | PHE B | 396 | 50.666 | 25.259 | 136.714 | 1.00 | 19.99 | B | C |
| ATOM | 2140 | CE1 | PHE B | 396 | 51.966 | 26.580 | 138.782 | 1.00 | 17.56 | B | C |
| ATOM | 2141 | CE2 | PHE B | 396 | 50.319 | 26.573 | 137.030 | 1.00 | 20.09 | B | C |
| ATOM | 2142 | CZ | PHE B | 396 | 50.974 | 27.236 | 138.070 | 1.00 | 19.48 | B | C |
| ATOM | 2143 | C | PHE B | 396 | 50.369 | 22.429 | 138.749 | 1.00 | 20.69 | B | C |
| ATOM | 2144 | O | PHE B | 396 | 50.257 | 22.821 | 139.916 | 1.00 | 20.46 | B | O |
| ATOM | 2145 | N | LEU B | 397 | 49.329 | 22.234 | 137.942 | 1.00 | 20.63 | B | N |
| ATOM | 2146 | CA | LEU B | 397 | 47.958 | 22.472 | 138.381 | 1.00 | 19.67 | B | C |
| ATOM | 2147 | CB | LEU B | 397 | 46.985 | 22.340 | 137.201 | 1.00 | 19.64 | B | C |
| ATOM | 2148 | CG | LEU B | 397 | 47.038 | 23.424 | 136.122 | 1.00 | 18.58 | B | C |
| ATOM | 2149 | CD1 | LEU B | 397 | −45.965 | 23.156 | 135.086 | 1.00 | 15.91 | B | C |
| ATOM | 2150 | CD2 | LEU B | 397 | 46.843 | 24.793 | 136.748 | 1.00 | 18.23 | B | C |
| ATOM | 2151 | C | LEU B | 397 | 47.512 | 21.550 | 139.510 | 1.00 | 18.44 | B | C |
| ATOM | 2152 | O | LEU B | 397 | 46.858 | 21.997 | 140.452 | 1.00 | 19.34 | B | O |
| ATOM | 2153 | N | VAL B | 398 | 47.846 | 20.266 | 139.417 | 1.00 | 17.99 | B | N |
| ATOM | 2154 | CA | VAL B | 398 | 47.464 | 19.326 | 140.466 | 1.00 | 19.04 | B | C |
| ATOM | 2155 | CB | VAL B | 398 | 47.733 | 17.866 | 140.043 | 1.00 | 19.35 | B | C |
| ATOM | 2156 | CG1 | VAL B | 398 | 47.353 | 16.905 | 141.173 | 1.00 | 15.55 | B | C |
| ATOM | 2157 | CG2 | VAL B | 398 | 46.913 | 17.538 | 138.809 | 1.00 | 19.62 | B | C |
| ATOM | 2158 | C | VAL B | 398 | 48.211 | 19.629 | 141.773 | 1.00 | 20.64 | B | C |
| ATOM | 2159 | O | VAL B | 398 | 47.659 | 19.469 | 142.862 | 1.00 | 21.46 | B | O |
| ATOM | 2160 | N | ALA B | 399 | 49.457 | 20.079 | 141.660 | 1.00 | 18.80 | B | N |
| ATOM | 2161 | CA | ALA B | 399 | 50.254 | 20.411 | 142.834 | 1.00 | 21.04 | B | C |
| ATOM | 2162 | CB | ALA B | 399 | 51.701 | 20.743 | 142.421 | 1.00 | 17.73 | B | C |
| ATOM | 2163 | C | ALA B | 399 | 49.636 | 21.599 | 143.566 | 1.00 | 21.74 | B | C |
| ATOM | 2164 | O | ALA B | 399 | 49.548 | 21.615 | 144.795 | 1.00 | 22.28 | B | O |
| ATOM | 2165 | N | ALA B | 400 | 49.210 | 22.597 | 142.802 | 1.00 | 21.01 | B | N |
| ATOM | 2166 | CA | ALA B | 400 | 48.611 | 23.787 | 143.378 | 1.00 | 20.55 | B | C |
| ATOM | 2167 | CB | ALA B | 400 | 48.270 | 24.788 | 142.274 | 1.00 | 19.74 | B | C |
| ATOM | 2168 | C | ALA B | 400 | 47.363 | 23.413 | 144.168 | 1.00 | 22.57 | B | C |
| ATOM | 2169 | O | ALA B | 400 | 47.090 | 23.992 | 145.220 | 1.00 | 23.97 | B | O |
| ATOM | 2170 | N | HIS B | 401 | 46.612 | 22.442 | 143.658 | 1.00 | 22.90 | B | N |
| ATOM | 2171 | CA | HIS B | 401 | 45.397 | 21.980 | 144.321 | 1.00 | 23.48 | B | C |
| ATOM | 2172 | CB | HIS B | 401 | 44.616 | 21.041 | 143.395 | 1.00 | 22.18 | B | C |
| ATOM | 2173 | CG | HIS B | 401 | 43.328 | 20.544 | 143.976 | 1.00 | 23.80 | B | C |
| ATOM | 2174 | CD2 | HIS B | 401 | 43.044 | 19.429 | 144.691 | 1.00 | 21.37 | B | C |
| ATOM | 2175 | ND1 | HIS B | 401 | 42.138 | 21.230 | 143.847 | 1.00 | 23.81 | B | N |
| ATOM | 2176 | CE1 | HIS B | 401 | 41.177 | 20.557 | 144.454 | 1.00 | 24.18 | B | C |
| ATOM | 2177 | NE2 | HIS B | 401 | 41.699 | 19.461 | 144.976 | 1.00 | 21.29 | B | N |
| ATOM | 2178 | C | HIS B | 401 | 45.763 | 21.246 | 145.615 | 1.00 | 24.30 | B | C |
| ATOM | 2179 | O | HIS B | 401 | 45.192 | 21.519 | 146.673 | 1.00 | 24.53 | B | O |
| ATOM | 2180 | N | GLU B | 402 | 46.714 | 20.317 | 145.529 | 1.00 | 23.82 | B | N |
| ATOM | 2181 | CA | GLU B | 402 | 47.150 | 19.566 | 146.708 | 1.00 | 22.77 | B | C |
| ATOM | 2182 | CB | GLU B | 402 | 48.217 | 18.530 | 146.341 | 1.00 | 22.35 | B | C |
| ATOM | 2183 | CG | GLU B | 402 | 47.744 | 17.435 | 145.406 | 1.00 | 21.55 | B | C |
| ATOM | 2184 | CD | GLU B | 402 | 46.298 | 17.071 | 145.636 | 1.00 | 21.21 | B | C |
| ATOM | 2185 | OE1 | GLU B | 402 | 45.914 | 16.813 | 146.790 | 1.00 | 23.03 | B | O |
| ATOM | 2186 | OE2 | GLU B | 402 | 45.539 | 17.043 | 144.654 | 1.00 | 26.51 | B | O |
| ATOM | 2187 | C | GLU B | 402 | 47.710 | 20.486 | 147.785 | 1.00 | 22.14 | B | C |
| ATOM | 2188 | O | GLU B | 402 | 47.431 | 20.301 | 148.966 | 1.00 | 24.45 | B | O |
| ATOM | 2189 | N | PHE B | 403 | 48.504 | 21.473 | 147.382 | 1.00 | 22.87 | B | N |
| ATOM | 2190 | CA | PHE B | 403 | 49.079 | 22.396 | 148.348 | 1.00 | 23.58 | B | C |
| ATOM | 2191 | CB | PHE B | 403 | 50.124 | 23.308 | 147.688 | 1.00 | 20.83 | B | C |
| ATOM | 2192 | CG | PHE B | 403 | 51.322 | 22.571 | 147.140 | 1.00 | 22.36 | B | C |
| ATOM | 2193 | CD1 | PHE B | 403 | 51.587 | 21.252 | 147.518 | 1.00 | 20.84 | B | C |
| ATOM | 2194 | CD2 | PHE B | 403 | 52.175 | 23.189 | 146.230 | 1.00 | 18.11 | B | C |
| ATOM | 2195 | CE1 | PHE B | 403 | 52.682 | 20.560 | 146.989 | 1.00 | 21.54 | B | C |
| ATOM | 2196 | CE2 | PHE B | 403 | 53.272 | 22.507 | 145.695 | 1.00 | 18.09 | B | C |
| ATOM | 2197 | CZ | PHE B | 403 | 53.525 | 21.193 | 146.072 | 1.00 | 19.86 | B | C |
| ATOM | 2198 | C | PHE B | 403 | 47.975 | 23.229 | 148.981 | 1.00 | 25.40 | B | C |
| ATOM | 2199 | O | PHE B | 403 | 48.155 | 23.801 | 150.055 | 1.00 | 27.88 | B | O |
| ATOM | 2200 | N | GLY B | 404 | 46.829 | 23.288 | 148.310 | 1.00 | 25.83 | B | N |
| ATOM | 2201 | CA | GLY B | 404 | 45.703 | 24.038 | 148.833 | 1.00 | 25.16 | B | C |
| ATOM | 2202 | C | GLY B | 404 | 45.208 | 23.328 | 150.076 | 1.00 | 25.03 | B | C |
| ATOM | 2203 | O | GLY B | 404 | 44.922 | 23.953 | 151.096 | 1.00 | 24.69 | B | O |
| ATOM | 2204 | N | HIS B | 405 | 45.110 | 22.008 | 149.980 | 1.00 | 23.03 | B | N |
| ATOM | 2205 | CA | HIS B | 405 | 44.689 | 21.186 | 151.102 | 1.00 | 23.37 | B | C |
| ATOM | 2206 | CB | HIS B | 405 | 44.537 | 19.731 | 150.668 | 1.00 | 21.33 | B | C |
| ATOM | 2207 | CG | HIS B | 405 | 43.325 | 19.474 | 149.834 | 1.00 | 26.14 | B | C |
| ATOM | 2208 | CD2 | HIS B | 405 | 43.164 | 18.781 | 148.683 | 1.00 | 23.33 | B | C |
| ATOM | 2209 | ND1 | HIS B | 405 | 42.076 | 19.944 | 150.178 | 1.00 | 25.46 | B | N |
| ATOM | 2210 | CE1 | HIS B | 405 | 41.198 | 19.551 | 149.274 | 1.00 | 26.71 | B | C |
| ATOM | 2211 | NE2 | HIS B | 405 | 41.833 | 18.844 | 148.357 | 1.00 | 25.66 | B | N |
| ATOM | 2212 | C | HIS B | 405 | 45.736 | 21.262 | 152.215 | 1.00 | 26.13 | B | C |
| ATOM | 2213 | O | HIS B | 405 | 45.407 | 21.507 | 153.371 | 1.00 | 27.66 | B | O |
| ATOM | 2214 | N | ALA B | 406 | 46.998 | 21.053 | 151.850 | 1.00 | 24.97 | B | N |
| ATOM | 2215 | CA | ALA B | 406 | 48.099 | 21.082 | 152.802 | 1.00 | 25.23 | B | C |
| ATOM | 2216 | CB | ALA B | 406 | 49.424 | 20.868 | 152.075 | 1.00 | 25.56 | B | C |
| ATOM | 2217 | C | ALA B | 406 | 48.150 | 22.374 | 153.606 | 1.00 | 24.96 | B | C |
| ATOM | 2218 | O | ALA B | 406 | 48.822 | 22.433 | 154.634 | 1.00 | 24.90 | B | O |
| ATOM | 2219 | N | LEU B | 407 | 47.450 | 23.407 | 153.139 | 1.00 | 25.75 | B | N |
| ATOM | 2220 | CA | LEU B | 407 | 47.424 | 24.683 | 153.849 | 1.00 | 26.02 | B | C |
| ATOM | 2221 | CB | LEU B | 407 | 47.613 | 25.856 | 152.887 | 1.00 | 24.31 | B | C |
| ATOM | 2222 | CG | LEU B | 407 | 48.951 | 25.959 | 152.146 | 1.00 | 27.21 | B | C |
| ATOM | 2223 | CD1 | LEU B | 407 | 48.960 | 27.227 | 151.301 | 1.00 | 24.05 | B | C |
| ATOM | 2224 | CD2 | LEU B | 407 | 50.110 | 25.979 | 153.132 | 1.00 | 25.82 | B | C |
| ATOM | 2225 | C | LEU B | 407 | 46.121 | 24.869 | 154.627 | 1.00 | 27.45 | B | C |
| ATOM | 2226 | O | LEU B | 407 | 45.947 | 25.868 | 155.323 | 1.00 | 28.27 | B | O |
| ATOM | 2227 | N | GLY B | 408 | 45.208 | 23.910 | 154.504 | 1.00 | 27.89 | B | N |
| ATOM | 2228 | CA | GLY B | 408 | 43.957 | 23.998 | 155.234 | 1.00 | 30.11 | B | C |
| ATOM | 2229 | C | GLY B | 408 | 42.673 | 24.146 | 154.437 | 1.00 | 31.82 | B | C |
| ATOM | 2230 | O | GLY B | 408 | 41.588 | 23.994 | 154.989 | 1.00 | 34.34 | B | O |
| ATOM | 2231 | N | LEU B | 409 | 42.772 | 24.438 | 153.147 | 1.00 | 33.15 | B | N |
| ATOM | 2232 | CA | LEU B | 409 | 41.575 | 24.601 | 152.325 | 1.00 | 32.57 | B | C |
| ATOM | 2233 | CB | LEU B | 409 | 41.925 | 25.260 | 150.991 | 1.00 | 31.21 | B | C |
| ATOM | 2234 | CG | LEU B | 409 | 42.400 | 26.711 | 151.009 | 1.00 | 32.82 | B | C |
| ATOM | 2235 | CD1 | LEU B | 409 | 42.551 | 27.189 | 149.581 | 1.00 | 31.72 | B | C |
| ATOM | 2236 | CD2 | LEU B | 409 | 41.397 | 27.589 | 151.747 | 1.00 | 33.90 | B | C |
| ATOM | 2237 | C | LEU B | 409 | 40.826 | 23.305 | 152.034 | 1.00 | 33.31 | B | C |
| ATOM | 2238 | O | LEU B | 409 | 41.427 | 22.250 | 151.850 | 1.00 | 32.51 | B | O |
| ATOM | 2239 | N | ASP B | 410 | 39.500 | 23.396 | 152.003 | 1.00 | 35.18 | B | N |
| ATOM | 2240 | CA | ASP B | 410 | 38.660 | 22.248 | 151.684 | 1.00 | 35.77 | B | C |
| ATOM | 2241 | CB | ASP B | 410 | 37.422 | 22.186 | 152.595 | 1.00 | 42.82 | B | C |
| ATOM | 2242 | CG | ASP B | 410 | 37.767 | 21.848 | 154.046 | 1.00 | 51.30 | B | C |
| ATOM | 2243 | OD1 | ASP B | 410 | 38.706 | 21.050 | 154.273 | 1.00 | 53.65 | B | O |
| ATOM | 2244 | OD2 | ASP B | 410 | 37.084 | 22.365 | 154.963 | 1.00 | 54.35 | B | O |
| ATOM | 2245 | C | ASP B | 410 | 38.213 | 22.438 | 150.232 | 1.00 | 34.97 | B | C |
| ATOM | 2246 | O | ASP B | 410 | 38.553 | 23.442 | 149.592 | 1.00 | 31.85 | B | O |
| ATOM | 2247 | N | HIS B | 411 | 37.4522 | 1.482 | 149.713 | 1.00 | 32.49 | B | N |
| ATOM | 2248 | CA | HIS B | 411 | 36.967 | 21.565 | 148.342 | 1.00 | 33.78 | B | C |
| ATOM | 2249 | CB | HIS B | 411 | 36.312 | 20.248 | 147.944 | 1.00 | 30.30 | B | C |
| ATOM | 2250 | CG | HIS B | 411 | 37.294 | 19.186 | 147.573 | 1.00 | 29.54 | B | C |
| ATOM | 2251 | CD2 | HIS B | 411 | 38.532 | 19.270 | 147.036 | 1.00 | 27.76 | B | C |
| ATOM | 2252 | ND1 | HIS B | 411 | 37.039 | 17.843 | 147.741 | 1.00 | 28.79 | B | N |
| ATOM | 2253 | CE1 | HIS B | 411 | 38.079 | 17.144 | 147.325 | 1.00 | 28.07 | B | C |
| ATOM | 2254 | NE2 | HIS B | 411 | 38.998 | 17.986 | 146.892 | 1.00 | 31.97 | B | N |
| ATOM | 2255 | C | HIS B | 411 | 35.985 | 22.704 | 148.128 | 1.00 | 35.95 | B | C |
| ATOM | 2256 | O | HIS B | 411 | 35.224 | 23.062 | 149.028 | 1.00 | 38.94 | B | O |
| ATOM | 2257 | N | SER B | 412 | 36.013 | 23.274 | 146.927 | 1.00 | 37.78 | B | N |
| ATOM | 2258 | CA | SER B | 412 | 35.115 | 24.365 | 146.566 | 1.00 | 38.30 | B | C |
| ATOM | 2259 | CB | SER B | 412 | 35.846 | 25.417 | 145.726 | 1.00 | 37.72 | B | C |
| ATOM | 2260 | OG | SER B | 412 | 34.953 | 26.426 | 145.281 | 1.00 | 34.94 | B | O |
| ATOM | 2261 | C | SER B | 412 | 33.957 | 23.800 | 145.763 | 1.00 | 39.44 | B | C |
| ATOM | 2262 | O | SER B | 412 | 34.050 | 22.703 | 145.213 | 1.00 | 38.62 | B | O |
| ATOM | 2263 | N | SER B | 413 | 32.862 | 24.553 | 145.715 | 1.00 | 41.65 | B | N |
| ATOM | 2264 | CA | SER B | 413 | 31.672 | 24.149 | 144.973 | 1.00 | 43.35 | B | C |
| ATOM | 2265 | CB | SER B | 413 | 30.414 | 24.431 | 145.806 | 1.00 | 43.75 | B | C |
| ATOM | 2266 | OG | SER B | 413 | 30.416 | 25.759 | 146.311 | 1.00 | 45.44 | B | O |
| ATOM | 2267 | C | SER B | 413 | 31.620 | 24.910 | 143.644 | 1.00 | 43.39 | B | C |
| ATOM | 2268 | O | SER B | 413 | 30.789 | 24.627 | 142.782 | 1.00 | 43.76 | B | O |
| ATOM | 2269 | N | VAL B | 414 | 32.520 | 25.878 | 143.495 | 1.00 | 43.36 | B | N |
| ATOM | 2270 | CA | VAL B | 414 | 32.613 | 26.679 | 142.282 | 1.00 | 45.01 | B | C |
| ATOM | 2271 | CB | VAL B | 414 | 33.330 | 28.015 | 142.569 | 1.00 | 43.92 | B | C |
| ATOM | 2272 | CG1 | VAL B | 414 | 33.395 | 28.862 | 141.307 | 1.00 | 42.90 | B | C |
| ATOM | 2273 | CG2 | VAL B | 414 | 32.606 | 28.761 | 143.691 | 1.00 | 42.04 | B | C |
| ATOM | 2274 | C | VAL B | 414 | 33.418 | 25.873 | 141.257 | 1.00 | 49.61 | B | C |
| ATOM | 2275 | O | VAL B | 414 | 34.639 | 25.731 | 141.384 | 1.00 | 50.35 | B | O |
| ATOM | 2276 | N | PRO B | 415 | 32.739 | 25.326 | 140.231 | 1.00 | 53.34 | B | N |
| ATOM | 2277 | CD | PRO B | 415 | 31.281 | 25.415 | 140.033 | 1.00 | 54.43 | B | C |
| ATOM | 2278 | CA | PRO B | 415 | 33.348 | 24.518 | 139.166 | 1.00 | 54.30 | B | C |
| ATOM | 2279 | CB | PRO B | 415 | 32.200 | 24.343 | 138.173 | 1.00 | 56.11 | B | C |
| ATOM | 2280 | CG | PRO B | 415 | 31.014 | 24.261 | 139.078 | 1.00 | 55.46 | B | C |
| ATOM | 2281 | C | PRO B | 415 | 34.614 | 25.060 | 138.505 | 1.00 | 52.96 | B | C |
| ATOM | 2282 | O | PRO B | 415 | 35.396 | 24.287 | 137.955 | 1.00 | 54.37 | B | O |
| ATOM | 2283 | N | GLU B | 416 | 34.818 | 26.373 | 138.544 | 1.00 | 50.37 | B | N |
| ATOM | 2284 | CA | GLU B | 416 | 36.016 | 26.946 | 137.940 | 1.00 | 48.82 | B | C |
| ATOM | 2285 | CB | GLU B | 416 | 35.662 | 28.135 | 137.041 | 1.00 | 53.26 | B | C |
| ATOM | 2286 | CG | GLU B | 416 | 34.714 | 29.151 | 137.651 | 1.00 | 58.55 | B | C |
| ATOM | 2287 | CD | GLU B | 416 | 33.266 | 28.900 | 137.270 | 1.00 | 61.82 | B | C |
| ATOM | 2288 | OE1 | GLU B | 416 | 32.399 | 29.699 | 137.684 | 1.00 | 62.91 | B | O |
| ATOM | 2289 | OE2 | GLU B | 416 | 32.994 | 27.907 | 136.556 | 1.00 | 63.31 | B | O |
| ATOM | 2290 | C | GLU B | 416 | 37.059 | 27.369 | 138.971 | 1.00 | 45.83 | B | C |
| ATOM | 2291 | O | GLU B | 416 | 3 8.023 | 28.060 | 138.646 | 1.00 | 45.77 | B | O |
| ATOM | 2292 | N | ALA B | 417 | 36.859 | 26.964 | 140.220 | 1.00 | 40.82 | B | N |
| ATOM | 2293 | CA | ALA B | 417 | 37.811 | 27.283 | 141.278 | 1.00 | 37.74 | B | C |
| ATOM | 2294 | CB | ALA B | 417 | 37.105 | 27.337 | 142.632 | 1.00 | 33.86 | B | C |
| ATOM | 2295 | C | ALA B | 417 | 38.867 | 26.181 | 141.281 | 1.00 | 36.14 | B | C |
| ATOM | 2296 | O | ALA B | 417 | 38.561 | 25.018 | 140.984 | 1.00 | 34.99 | B | O |
| ATOM | 2297 | N | LEU B | 418 | 40.107 | 26.543 | 141.605 | 1.00 | 34.03 | B | N |
| ATOM | 2298 | CA | LEU B | 418 | 41.191 | 25.569 | 141.648 | 1.00 | 30.71 | B | C |
| ATOM | 2299 | CB | LEU B | 418 | 42.502 | 26.248 | 142.048 | 1.00 | 31.54 | B | C |
| ATOM | 2300 | CG | LEU B | 418 | 43.718 | 25.338 | 142.279 | 1.00 | 30.96 | B | C |
| ATOM | 2301 | CD1 | LEU B | 418 | 44.048 | 24.554 | 141.013 | 1.00 | 28.41 | B | C |
| ATOM | 2302 | CD2 | LEU B | 418 | 44.908 | 26.190 | 142.704 | 1.00 | 29.00 | B | C |
| ATOM | 2303 | C | LEU B | 418 | 40.886 | 24.445 | 142.629 | 1.00 | 30.37 | B | C |
| ATOM | 2304 | O | LEU B | 418 | 41.261 | 23.295 | 142.402 | 1.00 | 31.03 | B | O |
| ATOM | 2305 | N | MET B | 419 | 40.197 | 24.772 | 143.719 | 1.00 | 29.50 | B | N |
| ATOM | 2306 | CA | MET B | 419 | 39.871 | 23.764 | 144.717 | 1.00 | 30.42 | B | C |
| ATOM | 2307 | CB | MET B | 419 | 39.657 | 24.418 | 146.085 | 1.00 | 31.33 | B | C |
| ATOM | 2308 | CG | MET B | 419 | 40.933 | 24.997 | 146.691 | 1.00 | 31.92 | B | C |
| ATOM | 2309 | SD | MET B | 419 | 42.360 | 23.881 | 146.543 | 1.00 | 33.33 | B | S |
| ATOM | 2310 | CE | MET B | 419 | 41.836 | 22.484 | 147.532 | 1.00 | 27.72 | B | C |
| ATOM | 2311 | C | MET B | 419 | 38.688 | 22.862 | 144.381 | 1.00 | 30.21 | B | C |
| ATOM | 2312 | O | MET B | 419 | 38.301 | 22.021 | 145.193 | 1.00 | 30.07 | B | O |
| ATOM | 2313 | N | TYR B | 420 | 38.119 | 23.026 | 143.190 | 1.00 | 31.40 | B | N |
| ATOM | 2314 | CA | TYR B | 420 | 37.006 | 22.180 | 142.769 | 1.00 | 31.95 | B | C |
| ATOM | 2315 | CB | TYR B | 420 | 36.550 | 22.570 | 141.356 | 1.00 | 36.57 | B | C |
| ATOM | 2316 | CG | TYR B | 420 | 35.227 | 21.951 | 140.953 | 1.00 | 41.51 | B | C |
| ATOM | 2317 | CD1 | TYR B | 420 | 34.035 | 22.330 | 141.577 | 1.00 | 43.15 | B | C |
| ATOM | 2318 | CE1 | TYR B | 420 | 32.824 | 21.719 | 141.250 | 1.00 | 43.91 | B | C |
| ATOM | 2319 | CD2 | TYR B | 420 | 35.173 | 20.947 | 139.986 | 1.00 | 42.71 | B | C |
| ATOM | 2320 | CE2 | TYR B | 420 | 33.971 | 20.330 | 139.655 | 1.00 | 43.77 | B | C |
| ATOM | 2321 | CZ | TYR B | 420 | 32.803 | 20.717 | 140.290 | 1.00 | 44.73 | B | C |
| ATOM | 2322 | OH | TYR B | 420 | 31.628 | 20.080 | 139.969 | 1.00 | 45.83 | B | O |
| ATOM | 2323 | C | TYR B | 420 | 37.558 | 20.749 | 142.790 | 1.00 | 31.80 | B | C |
| ATOM | 2324 | O | TYR B | 420 | 38.658 | 20.498 | 142.310 | 1.00 | 35.03 | B | O |
| ATOM | 2325 | N | PRO B | 421 | 36.797 | 19.792 | 143.340 | 1.00 | 31.81 | B | N |
| ATOM | 2326 | CD | PRO B | 421 | 35.430 | 20.002 | 143.846 | 1.00 | 31.47 | B | C |
| ATOM | 2327 | CA | PRO B | 421 | 37.177 | 18.376 | 143.455 | 1.00 | 31.76 | B | C |
| ATOM | 2328 | CB | PRO B | 421 | 36.033 | 17.783 | 144.277 | 1.00 | 32.37 | B | C |
| ATOM | 2329 | CG | PRO B | 421 | 34.869 | 18.597 | 143.833 | 1.00 | 31.84 | B | C |
| ATOM | 2330 | C | PRO B | 421 | 37.462 | 17.536 | 142.212 | 1.00 | 32.82 | B | C |
| ATOM | 2331 | O | PRO B | 421 | 37.890 | 16.383 | 142.329 | 1.00 | 33.44 | B | O |
| ATOM | 2332 | N | MET B | 422 | 37.225 | 18.078 | 141.028 | 1.00 | 34.03 | B | N |
| ATOM | 2333 | CA | MET B | 422 | 37.469 | 17.303 | 139.819 | 1.00 | 34.21 | B | C |
| ATOM | 2334 | CB | MET B | 422 | 36.156 | 17.127 | 139.050 | 1.00 | 40.49 | B | C |
| ATOM | 2335 | CG | MET B | 422 | 35.093 | 16.379 | 139.863 | 1.00 | 46.52 | B | C |
| ATOM | 2336 | SD | MET B | 422 | 33.548 | 16.039 | 138.978 | 1.00 | 54.83 | B | S |
| ATOM | 2337 | CE | MET B | 422 | 33.899 | 14.421 | 138.287 | 1.00 | 48.46 | B | C |
| ATOM | 2338 | C | MET B | 422 | 38.520 | 17.987 | 138.965 | 1.00 | 30.07 | B | C |
| ATOM | 2339 | O | MET B | 422 | 38.610 | 19.208 | 138.960 | 1.00 | 27.30 | B | O |
| ATOM | 2340 | N | TYR B | 423 | 39.328 | 17.205 | 138.256 | 1.00 | 30.57 | B | N |
| ATOM | 2341 | CA | TYR B | 423 | 40.386 | 17.789 | 137.423 | 1.00 | 30.47 | B | C |
| ATOM | 2342 | CB | TYR B | 423 | 41.432 | 16.730 | 137.066 | 1.00 | 28.25 | B | C |
| ATOM | 2343 | CG | TYR B | 423 | 42.541 | 17.261 | 136.179 | 1.00 | 28.83 | B | C |
| ATOM | 2344 | CD1 | TYR B | 423 | 43.549 | 18.077 | 136.695 | 1.00 | 26.05 | B | C |
| ATOM | 2345 | CE1 | TYR B | 423 | 44.547 | 18.607 | 135.866 | 1.00 | 26.97 | B | C |
| ATOM | 2346 | CD2 | TYR B | 423 | 42.557 | 16.984 | 134.809 | 1.00 | 29.17 | B | C |
| ATOM | 2347 | CE2 | TYR B | 423 | 43.548 | 17.509 | 133.973 | 1.00 | 28.79 | B | C |
| ATOM | 2348 | CZ | TYR B | 423 | 44.537 | 18.319 | 134.508 | 1.00 | 27.46 | B | C |
| ATOM | 2349 | OH | TYR B | 423 | 45.499 | 18.848 | 133.679 | 1.00 | 29.00 | B | O |
| ATOM | 2350 | C | TYR B | 423 | 39.891 | 18.450 | 136.132 | 1.00 | 29.81 | B | C |
| ATOM | 2351 | O | TYR B | 423 | 38.997 | 17.941 | 135.456 | 1.00 | 28.16 | B | O |
| ATOM | 2352 | N | ALA B | 424 | 40.496 | 19.583 | 135.795 | 1.00 | 30.84 | B | N |
| ATOM | 2353 | CA | ALA B | 424 | 40.155 | 20.320 | 134.582 | 1.00 | 30.74 | B | C |
| ATOM | 2354 | CB | ALA B | 424 | 38.891 | 21.137 | 134.809 | 1.00 | 30.80 | B | C |
| ATOM | 2355 | C | ALA B | 424 | 41.316 | 21.241 | 134.201 | 1.00 | 31.98 | B | C |
| ATOM | 2356 | O | ALA B | 424 | 41.662 | 22.147 | 134.960 | 1.00 | 30.87 | B | O |
| ATOM | 2357 | N | PHE B | 425 | 41.916 | 21.016 | 133.030 | 1.00 | 32.65 | B | N |
| ATOM | 2358 | CA | PHE B | 425 | 43.037 | 21.847 | 132.587 | 1.00 | 32.21 | B | C |
| ATOM | 2359 | CB | PHE B | 425 | 43.761 | 21.219 | 131.387 | 1.00 | 29.42 | B | C |
| ATOM | 2360 | CG | PHE B | 425 | 44.871 | 22.085 | 130.837 | 1.00 | 28.07 | B | C |
| ATOM | 2361 | CD1 | PHE B | 425 | 44.635 | 22.967 | 129.783 | 1.00 | 26.46 | B | C |
| ATOM | 2362 | CD2 | PHE B | 425 | 46.130 | 22.075 | 131.427 | 1.00 | 26.10 | B | C |
| ATOM | 2363 | CE1 | PHE B | 425 | 45.636 | 23.828 | 129.334 | 1.00 | 26.71 | B | C |
| ATOM | 2364 | CE2 | PHE B | 425 | 47.138 | 22.933 | 130.986 | 1.00 | 23.65 | B | C |
| ATOM | 2365 | CZ | PHE B | 425 | 46.890 | 23.811 | 129.941 | 1.00 | 24.65 | B | C |
| ATOM | 2366 | C | PHE B | 425 | 42.612 | 23.261 | 132.226 | 1.00 | 32.08 | B | C |
| ATOM | 2367 | O | PHE B | 425 | 41.517 | 23.474 | 131.723 | 1.00 | 35.15 | B | 0 |
| ATOM | 2368 | N | THR B | 426 | 43.489 | 24.226 | 132.480 | 1.00 | 32.53 | B | N |
| ATOM | 2369 | CA | THR B | 426 | 43.193 | 25.618 | 132.170 | 1.00 | 31.99 | B | C |
| ATOM | 2370 | CB | THR B | 426 | 42.377 | 26.297 | 133.309 | 1.00 | 33.76 | B | C |
| ATOM | 2371 | OG1 | THR B | 426 | 42.153 | 27.674 | 132.979 | 1.00 | 35.23 | B | O |
| ATOM | 2372 | CG2 | THR B | 426 | 43.132 | 26.236 | 134.638 | 1.00 | 32.87 | B | C |
| ATOM | 2373 | C | THR B | 426 | 44.470 | 26.415 | 131.960 | 1.00 | 32.40 | B | |
| ATOM | 2374 | O | THR B | 426 | 45.515 | 26.082 | 132.515 | 1.00 | 31.41 | B | O |
| ATOM | 2375 | N | GLU B | 427 | 44.376 | 27.458 | 131.143 | 1.00 | 32.69 | B | N |
| ATOM | 2376 | CA | GLU B | 427 | 45.505 | 28.338 | 130.877 | 1.00 | 34.12 | B | C |
| ATOM | 2377 | CB | GLU B | 427 | 45.593 | 28.670 | 129.384 | 1.00 | 36.51 | B | C |
| ATOM | 2378 | CG | GLU B | 427 | 45.967 | 27.493 | 128.490 | 1.00 | 41.69 | B | C |
| ATOM | 2379 | CD | GLU B | 427 | 46.103 | 27.888 | 127.024 | 1.00 | 45.50 | B | C |
| ATOM | 2380 | OE1 | GLU B | 427 | 45.082 | 28.269 | 126.410 | 1.00 | 44.55 | B | O |
| ATOM | 2381 | OE2 | GLU B | 427 | 47.235 | 27.822 | 126.488 | 1.00 | 47.29 | B | O |
| ATOM | 2382 | C | GLU B | 427 | 45.237 | 29.605 | 131.680 | 1.00 | 33.67 | B | C |
| ATOM | 2383 | O | GLU B | 427 | 44.133 | 29.793 | 132.186 | 1.00 | 35.20 | B | O |
| ATOM | 2384 | N | GLY B | 428 | 46.234 | 30.471 | 131.800 | 1.00 | 33.34 | B | N |
| ATOM | 2385 | CA | GLY B | 428 | 46.041 | 31.692 | 132.561 | 1.00 | 34.51 | B | C |
| ATOM | 2386 | C | GLY B | 428 | 46.066 | 31.428 | 134.057 | 1.00 | 36.70 | B | C |
| ATOM | 2387 | O | GLY B | 428 | 46.241 | 30.281 | 134.475 | 1.00 | 35.95 | B | O |
| ATOM | 2388 | N | PRO B | 429 | 45.887 | 32.468 | 134.893 | 1.00 | 37.61 | B | N |
| ATOM | 2389 | CD | PRO B | 429 | 45.427 | 33.803 | 134.473 | 1.00 | 37.68 | B | C |
| ATOM | 2390 | CA | PRO B | 429 | 45.888 | 32.363 | 136.361 | 1.00 | 35.27 | B | C |
| ATOM | 2391 | CB | PRO B | 429 | 45.444 | 33.753 | 136.806 | 1.00 | 36.80 | B | C |
| ATOM | 2392 | CG | PRO B | 429 | 44.594 | 34.224 | 135.655 | 1.00 | 38.82 | B | C |
| ATOM | 2393 | C | PRO B | 429 | 44.969 | 3 1.258 | 136.882 | 1.00 | 34.51 | B | C |
| ATOM | 2394 | O | PRO B | 429 | 43.769 | 31.248 | 136.605 | 1.00 | 35.20 | B | O |
| ATOM | 2395 | N | PRO B | 430 | 45.533 | 30.312 | 137.648 | 1.00 | 32.84 | B | N |
| ATOM | 2396 | CD | PRO B | 430 | 46.984 | 30.207 | 137.866 | 1.00 | 30.88 | B | C |
| ATOM | 2397 | CA | PRO B | 430 | 44.843 | 29.160 | 138.246 | 1.00 | 33.23 | B | C |
| ATOM | 2398 | CB | PRO B | 430 | 45.997 | 28.284 | 138.747 | 1.00 | 32.15 | B | C |
| ATOM | 2399 | CG | PRO B | 430 | 47.172 | 28.722 | 137.930 | 1.00 | 32.06 | B | C |
| ATOM | 2400 | C | PRO B | 430 | 43.870 | 29.482 | 139.381 | 1.00 | 33.86 | B | C |
| ATOM | 2401 | O | PRO B | 430 | 42.786 | 28.891 | 139.475 | 1.00 | 32.09 | B | O |
| ATOM | 2402 | N | LEU B | 431 | 44.271 | 30.405 | 140.250 | 1.00 | 34.56 | B | N |
| ATOM | 2403 | CA | LEU B | 431 | 43.456 | 30.778 | 141.401 | 1.00 | 35.70 | B | C |
| ATOM | 2404 | CB | LEU B | 431 | 44.300 | 31.550 | 142.419 | 1.00 | 33.79 | B | C |
| ATOM | 2405 | CG | LEU B | 431 | 45.449 | 30.793 | 143.080 | 1.00 | 33.47 | B | C |
| ATOM | 2406 | CD1 | LEU B | 431 | 46.049 | 31.651 | 144.190 | 1.00 | 33.46 | B | C |
| ATOM | 2407 | CD2 | LEU B | 431 | 44.934 | 29.481 | 143.649 | 1.00 | 36.47 | B | C |
| ATOM | 2408 | C | LEU B | 431 | 42.207 | 31.588 | 141.084 | 1.00 | 36.24 | B | C |
| ATOM | 2409 | O | LEU B | 431 | 42.271 | 32.66 | 1140.479 | 1.00 | 36.37 | B | O |
| ATOM | 2410 | N | HIS B | 432 | 41.064 | 31.064 | 141.505 | 1.00 | 36.71 | B | N |
| ATOM | 2411 | CA | HIS B | 432 | 39.811 | 31.760 | 141.299 | 1.00 | 41.25 | B | C |
| ATOM | 2412 | CB | HIS B | 432 | 38.662 | 30.761 | 141.191 | 1.00 | 40.99 | B | C |
| ATOM | 2413 | GG | HIS B | 432 | 37.332 | 31.404 | 140.963 | 1.00 | 44.68 | B | C |
| ATOM | 2414 | CD2 | HIS B | 432 | 36.589 | 31.541 | 139.838 | 1.00 | 44.42 | B | C |
| ATOM | 2415 | ND1 | HIS B | 432 | 36.626 | 32.031 | 141.967 | 1.00 | 46.47 | B | N |
| ATOM | 2416 | CE1 | HIS B | 432 | 35.504 | 32.524 | 141.472 | 1.00 | 45.59 | B | C |
| ATOM | 2417 | NE2 | HIS B | 432 | 35.459 | 32.241 | 140.182 | 1.00 | 46.62 | B | N |
| ATOM | 2418 | C | HIS B | 432 | 39.613 | 32.670 | 142.507 | 1.00 | 44.37 | B | C |
| ATOM | 2419 | O | HIS B | 432 | 40.151 | 32.400 | 143.588 | 1.00 | 46.02 | B | O |
| ATOM | 2420 | N | LYS B | 433 | 38.859 | 33.751 | 142.330 | 1.00 | 46.58 | B | N |
| ATOM | 2421 | CA | LYS B | 433 | 38.627 | 34.678 | 143.428 | 1.00 | 46.70 | B | C |
| ATOM | 2422 | CB | LYS B | 433 | 37.717 | 35.822 | 142.972 | 1.00 | 51.43 | B | C |
| ATOM | 2423 | CG | LYS B | 433 | 38.355 | 36.668 | 141.865 | 1.00 | 55.90 | B | C |
| ATOM | 2424 | CD | LYS B | 433 | 37.589 | 37.956 | 141.584 | 1.00 | 59.77 | B | C |
| ATOM | 2425 | CE | LYS B | 433 | 38.320 | 38.811 | 140.549 | 1.00 | 61.80 | B | C |
| ATOM | 2426 | NZ | LYS B | 433 | 37.720 | 40.171 | 140.380 | 1.00 | 62.64 | B | N |
| ATOM | 2427 | C | LYS B | 433 | 38.062 | 33.973 | 144.658 | 1.00 | 43.89 | B | C |
| ATOM | 2428 | O | LYS B | 433 | 38.150 | 34.491 | 145.767 | 1.00 | 44.99 | B | O |
| ATOM | 2429 | N | ASP B | 434 | 37.501 | 32.783 | 144.460 | 1.00 | 40.87 | B | N |
| ATOM | 2430 | CA | ASP B | 434 | 36.964 | 31.998 | 145.565 | 1.00 | 39.24 | B | C |
| ATOM | 2431 | CB | ASP B | 434 | 36.032 | 30.905 | 145.038 | 1.00 | 38.77 | B | C |
| ATOM | 2432 | CG | ASP B | 434 | 35.520 | 29.999 | 146.138 | 1.00 | 39.93 | B | C |
| ATOM | 2433 | OD1 | ASP B | 434 | 36.053 | 28.882 | 146.286 | 1.00 | 43.55 | B | O |
| ATOM | 2434 | OD2 | ASP B | 434 | 34.593 | 30.408 | 146.866 | 1.00 | 42.42 | B | O |
| ATOM | 2435 | C | ASP B | 434 | 38.127 | 31.365 | 146.337 | 1.00 | 40.97 | B | C |
| ATOM | 2436 | O | ASP B | 434 | 38.102 | 31.288 | 147.571 | 1.00 | 41.38 | B | O |
| ATOM | 2437 | N | ASP B | 435 | 39.144 | 30.915 | 145.601 | 1.00 | 39.44 | B | N |
| ATOM | 2438 | CA | ASP B | 435 | 40.324 | 30.305 | 146.204 | 1.00 | 37.18 | B | C |
| ATOM | 2439 | CB | ASP B | 435 | 41.258 | 29.737 | 145.128 | 1.00 | 36.04 | B | C |
| ATOM | 2440 | CG | ASP B | 435 | 40.608 | 28.653 | 144.293 | 1.00 | 37.10 | B | C |
| ATOM | 2441 | OD1 | ASP B | 435 | 40.086 | 27.671 | 144.868 | 1.00 | 35.74 | B | O |
| ATOM | 2442 | OD2 | ASP B | 435 | 40.632 | 28.779 | 143.050 | 1.00 | 40.79 | B | O |
| ATOM | 2443 | C | ASP B | 435 | 41.088 | 31.354 | 147.007 | 1.00 | 38.01 | B | C |
| ATOM | 2444 | O | ASP B | 435 | 41.591 | 31.077 | 148.098 | 1.00 | 39.15 | B | O |
| ATOM | 2445 | N | VAL B | 436 | 41.178 | 32.560 | 146.459 | 1.00 | 37.34 | B | N |
| ATOM | 2446 | CA | VAL B | 436 | 41.893 | 33.637 | 147.127 | 1.00 | 37.88 | B | C |
| ATOM | 2447 | CB | VAL B | 436 | 42.018 | 34.863 | 146.209 | 1.00 | 37.00 | B | C |
| ATOM | 2448 | CG1 | VAL B | 436 | 42.850 | 35.939 | 146.887 | 1.00 | 37.16 | B | C |
| ATOM | 2449 | CG2 | VAL B | 436 | 42.654 | 34.454 | 144.893 | 1.00 | 37.45 | B | C |
| ATOM | 2450 | C | VAL B | 436 | 41.203 | 34.051 | 148.426 | 1.00 | 41.35 | B | C |
| ATOM | 2451 | O | VAL B | 436 | 41.870 | 34.444 | 149.392 | 1.00 | 40.58 | B | O |
| ATOM | 2452 | N | ASN B | 437 | 39.872 | 33.964 | 148.445 | 1.00 | 42.61 | B | N |
| ATOM | 2453 | CA | ASN B | 437 | 39.103 | 34.323 | 149.633 | 1.00 | 42.98 | B | C |
| ATOM | 2454 | CB | ASN B | 437 | 37.602 | 34.372 | 149.331 | 1.00 | 44.38 | B | C |
| ATOM | 2455 | CG | ASN B | 437 | 37.199 | 35.607 | 148.564 | 1.00 | 46.07 | B | C |
| ATOM | 2456 | OD1 | ASN B | 437 | 37.778 | 36.681 | 148.743 | 1.00 | 49.54 | B | O |
| ATOM | 2457 | ND2 | ASN B | 437 | 36.182 | 35.471 | 147.721 | 1.00 | 46.44 | B | N |
| ATOM | 2458 | C | ASN B | 437 | 39.341 | 33.309 | 150.732 | 1.00 | 41.97 | B | C |
| ATOM | 2459 | O | ASN B | 437 | 39.656 | 33.665 | 151.863 | 1.00 | 44.18 | B | O |
| ATOM | 2460 | N | GLY B | 438 | 39.177 | 32.038 | 150.391 | 1.00 | 42.23 | B | N |
| ATOM | 2461 | CA | GLY B | 438 | 39.373 | 30.980 | 151.364 | 1.00 | 41.20 | B | C |
| ATOM | 2462 | C | GLY B | 438 | 40.737 | 31.015 | 152.030 | 1.00 | 40.27 | B | C |
| ATOM | 2463 | O | GLY B | 438 | 40.851 | 30.821 | 153.241 | 1.00 | 40.43 | B | O |
| ATOM | 2464 | N | ILE B | 439 | 41.778 | 31.269 | 151.246 | 1.00 | 39.21 | B | N |
| ATOM | 2465 | CA | ILE B | 439 | 43.125 | 31.305 | 151.796 | 1.00 | 38.89 | B | C |
| ATOM | 2466 | CB | ILE B | 439 | 44.195 | 31.186 | 150.666 | 1.00 | 34.85 | B | C |
| ATOM | 2467 | CG2 | ILE B | 439 | 44.318 | 32.488 | 149.913 | 1.00 | 32.27 | B | C |
| ATOM | 2468 | CG1 | ILE B | 439 | 45.546 | 30.783 | 151.262 | 1.00 | 34.00 | B | C |
| ATOM | 2469 | CD1 | ILE B | 439 | 45.558 | 29.373 | 151.853 | 1.00 | 32.09 | B | C |
| ATOM | 2470 | C | ILE B | 439 | 43.317 | 32.597 | 152.587 | 1.00 | 40.47 | B | C |
| ATOM | 2471 | O | ILE B | 439 | 44.034 | 32.625 | 153.588 | 1.00 | 41.20 | B | O |
| ATOM | 2472 | N | ARG B | 440 | 42.654 | 33.658 | 152.134 | 1.00 | 43.75 | B | N |
| ATOM | 2473 | CA | ARG B | 440 | 42.725 | 34.967 | 152.782 | 1.00 | 44.46 | B | C |
| ATOM | 2474 | CB | ARG B | 440 | 42.048 | 36.030 | 151.909 | 1.00 | 46.94 | B | C |
| ATOM | 2475 | CG | ARG B | 440 | 42.937 | 37.185 | 151.471 | 1.00 | 49.56 | B | C |
| ATOM | 2476 | CD | ARG B | 440 | 43.845 | 36.769 | 150.334 | 1.00 | 54.73 | B | C |
| ATOM | 2477 | NE | ARG B | 440 | 44.726 | 37.850 | 149.901 | 1.00 | 58.03 | B | N |
| ATOM | 2478 | CZ | ARG B | 440 | 44.306 | 38.991 | 149.362 | 1.00 | 60.42 | B | C |
| ATOM | 2479 | NH1 | ARG B | 440 | 43.009 | 39.211 | 149.186 | 1.00 | 60.93 | B | N |
| ATOM | 2480 | NH2 | ARG B | 440 | 45.188 | 39.910 | 148.988 | 1.00 | 60.50 | B | N |
| ATOM | 2481 | C | ARG B | 440 | 42.006 | 34.908 | 154.128 | 1.00 | 44.17 | B | C |
| ATOM | 2482 | O | ARG B | 440 | 42.320 | 35.666 | 155.047 | 1.00 | 43.24 | B | O |
| ATOM | 2483 | N | HIS B | 441 | 41.033 | 34.004 | 154.221 | 1.00 | 42.88 | B | N |
| ATOM | 2484 | CA | HIS B | 441 | 40.235 | 33.818 | 155.428 | 1.00 | 43.46 | B | C |
| ATOM | 2485 | CB | HIS B | 441 | 38.997 | 32.972 | 155.112 | 1.00 | 46.73 | B | C |
| ATOM | 2486 | CG | HIS B | 441 | 38.118 | 32.713 | 156.297 | 1.00 | 52.22 | B | C |
| ATOM | 2487 | CD2 | HIS B | 441 | 37.906 | 31.588 | 157.022 | 1.00 | 53.91 | B | C |
| ATOM | 2488 | ND1 | HIS B | 441 | 37.331 | 33.690 | 156.871 | 1.00 | 54.78 | B | N |
| ATOM | 2489 | CE1 | HIS B | 441 | 36.673 | 33.178 | 157.896 | 1.00 | 54.20 | B | C |
| ATOM | 2490 | NB2 | HIS B | 441 | 37.005 | 31.904 | 158.010 | 1.00 | 54.76 | B | N |
| ATOM | 2491 | C | HIS B | 441 | 41.029 | 33.144 | 156.538 | 1.00 | 42.38 | B | C |
| ATOM | 2492 | O | HIS B | 441 | 40.835 | 33.448 | 157.713 | 1.00 | 43.43 | B | O |
| ATOM | 2493 | N | LEU B | 442 | 41.924 | 32.233 | 156.158 | 1.00 | 40.54 | B | N |
| ATOM | 2494 | CA | LEU B | 442 | 42.747 | 31.495 | 157.118 | 1.00 | 37.84 | B | C |
| ATOM | 2495 | CB | LEU B | 442 | 43.136 | 30.124 | 156.542 | 1.00 | 36.23 | B | C |
| ATOM | 2496 | CG | LEU B | 442 | 42.020 | 29.121 | 156.224 | 1.00 | 36.95 | B | C |
| ATOM | 2497 | CD1 | LEU B | 442 | 42.600 | 27.921 | 155.494 | 1.00 | 37.45 | B | C |
| ATOM | 2498 | CD2 | LEU B | 442 | 41.337 | 28.676 | 157.507 | 1.00 | 38.67 | B | C |
| ATOM | 2499 | C | LEU B | 442 | 44.012 | 32.232 | 157.548 | 1.00 | 35.29 | B | C |
| ATOM | 2500 | O | LEU B | 442 | 44.367 | 32.218 | 158.721 | 1.00 | 37.44 | B | O |
| ATOM | 2501 | N | TYR B | 443 | 44.693 | 32.872 | 156.605 | 1.00 | 34.38 | B | N |
| ATOM | 2502 | CA | TYR B | 443 | 45.932 | 33.581 | 156.920 | 1.00 | 36.23 | B | C |
| ATOM | 2503 | CB | TYR B | 443 | 47.105 | 32.984 | 156.123 | 1.00 | 34.25 | B | C |
| ATOM | 2504 | CG | TYR B | 443 | 47.261 | 31.490 | 156.300 | 1.00 | 31.93 | B | C |
| ATOM | 2505 | CD1 | TYR B | 443 | 46.614 | 30.596 | 155.446 | 1.00 | 29.81 | B | C |
| ATOM | 2506 | CE1 | TYR B | 443 | 46.695 | 29.215 | 155.649 | 1.00 | 28.03 | B | C |
| ATOM | 2507 | CD2 | TYR B | 443 | 48.004 | 30.968 | 157.366 | 1.00 | 29.17 | B | C |
| ATOM | 2508 | CE2 | TYR B | 443 | 48.092 | 29.592 | 157.578 | 1.00 | 28.10 | B | C |
| ATOM | 2509 | CZ | TYR B | 443 | 47.433 | 28.721 | 156.718 | 1.00 | 29.15 | B | C |
| ATOM | 2510 | OH | TYR B | 443 | 47.496 | 27.360 | 156.932 | 1.00 | 27.85 | B | O |
| ATOM | 2511 | C | TYR B | 443 | 45.855 | 35.082 | 156.659 | 1.00 | 38.24 | B | C |
| ATOM | 2512 | O | TYR B | 443 | 46.801 | 35.794 | 157.063 | 1.00 | 40.58 | B | O |
| ATOM | 2513 | OT | TYR B | 443 | 44.864 | 35.527 | 156.044 | 1.00 | 41.15 | B | O |
| ATOM | 2562 | ZN | ZN B | 1 | 41.275 | 17.840 | 146.537 | 1.00 | 30.01 | Z | ZN |
| ATOM | 2563 | ZN | ZN B | 2 | 49.676 | 8.887 | 150.296 | 1.00 | 22.73 | Z | ZN |
| ATOM | 2570 | CA | CA B | 3 | 54.013 | 17.086 | 157.486 | 1.00 | 25.83 | C | C |
| ATOM | 2571 | CA | CA B | 4 | 54.046 | 8.303 | 135.827 | 1.00 | 47.76 | C | C |
| ATOM | 2572 | CA | CA B | 5 | 50.701 | 18.541 | 128.694 | 1.00 | 57.49 | C | C |
| ATOM | 2569 | CA | CA B | 6 | 45.250 | 7.241 | 139.948 | 1.00 | 21.01 | C | C |
| ATOM | 2574 | CA | CA B | 7 | 53.492 | 38.165 | 150.097 | 1.00 | 99.62 | C | C |
| ATOM | 2537 | C1 | FRA B | 2 | 40.988 | 17.843 | 141.282 | 1.00 | 21.42 | M | C |
| ATOM | 2538 | C2 | FRA B | 2 | 42.202 | 16.892 | 141.164 | 1.00 | 23.04 | M | C |
| ATOM | 2539 | C3 | FRA B | 2 | 43.397 | 17.813 | 140.945 | 1.00 | 21.68 | M | C |
| ATOM | 2540 | C4 | FRA B | 2 | 42.395 | 16.088 | 142.486 | 1.00 | 20.53 | M | C |
| ATOM | 2541 | C5 | FRA B | 2 | 41.287 | 15.047 | 142.944 | 1.00 | 22.68 | M | C |
| ATOM | 2542 | C6 | FRA B | 2 | −41.674 | 14.384 | 144.328 | 1.00 | 21.04 | M | C |
| ATOM | 2543 | N7 | FRA B | 2 | 41.894 | 15.346 | 145.441 | 1.00 | 26.66 | M | N |
| ATOM | 2544 | O8 | FRA B | 2 | 40.878 | 16.003 | 145.917 | 1.00 | 16.04 | M | O |
| ATOM | 2545 | C9 | FRA B | 2 | 43.101 | 15.578 | 145.982 | 1.00 | 25.43 | M | C |
| ATOM | 2546 | O10 | FRA B | 2 | 43.304 | 16.343 | 146.865 | 1.00 | 27.78 | M | O |
| ATOM | 2547 | C11 | FRA B | 2 | 41.103 | 13.852 | 141.935 | 1.00 | 24.21 | M | C |
| ATOM | 2548 | N12 | FRA B | 2 | 39.910 | 13.731 | 141.312 | 1.00 | 24.87 | M | N |
| ATOM | 2549 | C13 | FRA B | 2 | 39.558 | 12.636 | 140.307 | 1.00 | 25.69 | M | C |
| ATOM | 2550 | C14 | FRA B | 2 | 38.064 | 12.068 | 140.550 | 1.00 | 26.41 | M | C |
| ATOM | 2551 | C15 | FRA B | 2 | 37.671 | 10.934 | 139.534 | 1.00 | 23.54 | M | C |
| ATOM | 2552 | C16 | FRA B | 2 | 36.936 | 13.161 | 140.404 | 1.00 | 25.14 | M | C |
| ATOM | 2553 | C17 | FRA B | 2 | 37.918 | 11.501 | 141.991 | 1.00 | 25.52 | M | C |
| ATOM | 2554 | C18 | FRA B | 2 | 39.789 | 13.241 | 138.843 | 1.00 | 26.03 | M | C |
| ATOM | 2555 | O19 | FRA B | 2 | 39.414 | 14.401 | 138.550 | 1.00 | 27.42 | M | O |
| ATOM | 2556 | N20 | FRA B | 2 | 40.416 | 12.393 | 137.981 | 1.00 | 25.15 | M | N |
| ATOM | 2557 | C21 | FRA B | 2 | 40.752 | 12.702 | 136.573 | 1.00 | 30.05 | M | C |
| ATOM | 2558 | O22 | FRA B | 2 | 42.049 | 13.051 | 141.732 | 1.00 | 25.04 | M | O |
| ATOM | 2576 | O | HOH W | 1 | 53.372 | 24.644 | 125.318 | 1.00 | 1.00 | W | O |
| ATOM | 2577 | O | HOH W | 2 | 74.083 | 20.414 | 120.913 | 1.00 | 21.76 | W | O |
| ATOM | 2578 | O | HOH W | 3 | 49.364 | 12.911 | 104.335 | 1.00 | 26.01 | W | O |
| ATOM | 2579 | O | HOH W | 4 | 52.144 | 16.494 | 124.475 | 1.00 | 14.30 | W | O |
| ATOM | 2580 | O | HOH W | 5 | 58.587 | 7.0141 | 15.292 | 1.00 | 27.11 | W | O |
| ATOM | 2581 | O | HOH W | 6 | 75.605 | 18.864 | 112.249 | 1.00 | 18.56 | W | O |
| ATOM | 2582 | O | HOH W | 7 | 65.295 | 7.257 | 120.405 | 1.00 | 1.00 | W | O |
| ATOM | 2583 | O | HOH W | 8 | 57.185 | 8.107 | 113.191 | 1.00 | 17.03 | W | O |
| ATOM | 2584 | O | HON W | 9 | 64.469 | 12.077 | 98.818 | 1.00 | 47.82 | W | O |
| ATOM | 2585 | O | HOH W | 10 | 51.666 | 21.908 | 124.829 | 1.00 | 15.33 | W | O |
| ATOM | 2586 | O | HOH W | 11 | 67.629 | 24.639 | 127.665 | 1.00 | 20.55 | W | O |
| ATOM | 2587 | O | HOH W | 12 | 67.365 | 14.751 | 108.625 | 1.00 | 21.02 | W | O |
| ATOM | 2588 | O | HOH W | 13 | 52.957 | 12.041 | 116.137 | 1.00 | 15.20 | W | O |
| ATOM | 2589 | O | HOH W | 14 | 51.253 | 9.646 | 116.663 | 1.00 | 29.59 | W | O |
| ATOM | 2590 | O | HOH W | 15 | 48.380 | 16.148 | 113.137 | 1.00 | 26.43 | W | O |
| ATOM | 2591 | O | HOH W | 16 | 49.177 | 9.855 | 111.408 | 1.00 | 25.21 | W | O |
| ATOM | 2592 | O | HOH W | 17 | 56.870 | 18.582 | 90.415 | 1.00 | 50.62 | W | O |
| ATOM | 2593 | O | HOH W | 18 | 56.995 | 17.921 | 96.140 | 1.00 | 26.85 | W | O |
| ATOM | 2594 | O | HOH W | 19 | 61.756 | 9.877 | 125.515 | 1.00 | 40.50 | W | O |
| ATOM | 2595 | O | HOH W | 20 | 45.873 | 28.448 | 97.477 | 1.00 | 25.98 | W | O |
| ATOM | 2596 | O | HOH W | 21 | 50.275 | 10.441 | 122.477 | 1.00 | 33.64 | W | O |
| ATOM | 2597 | O | HOH W | 22 | 56.828 | 39.659 | 107.746 | 1.00 | 37.53 | W | O |
| ATOM | 2598 | O | HOH W | 23 | 56.922 | 8.730 | 101.984 | 1.00 | 51.25 | W | O |
| ATOM | 2599 | O | HOH W | 24 | 63.090 | 12.420 | 103.873 | 1.00 | 23.17 | W | O |
| ATOM | 2600 | O | HOH W | 25 | 78.432 | 18.341 | 111.530 | 1.00 | 33.10 | W | O |
| ATOM | 2601 | O | HOH W | 26 | 55.278 | 7.063 | 126.543 | 1.00 | 22.51 | W | O |
| ATOM | 2602 | O | HOH W | 27 | 71.699 | 14.944 | 110.281 | 1.00 | 31.13 | W | O |
| ATOM | 2603 | O | HOH W | 28 | 65.856 | 12.461 | 105.207 | 1.00 | 34.23 | W | O |
| ATOM | 2604 | O | HOH W | 29 | 49.226 | 13.404 | 112.556 | 1.00 | 36.10 | W | O |
| ATOM | 2605 | O | HOH W | 30 | 45.806 | 26.797 | 113.582 | 1.00 | 25.06 | W | O |
| ATOM | 2606 | O | HOH W | 31 | 50.921 | 22.489 | 96.419 | 1.00 | 38.17 | W | O |
| ATOM | 2607 | O | HOH W | 32 | 47.043 | 17.179 | 110.619 | 1.00 | 27.88 | W | O |
| ATOM | 2608 | O | HOH W | 33 | 50.505 | 31.197 | 136.475 | 1.00 | 33.10 | W | O |
| ATOM | 2609 | O | HOH W | 34 | 47.341 | 22.271 | 121.211 | 1.00 | 20.31 | W | O |
| ATOM | 2610 | O | HOH W | 35 | 61.912 | 15.434 | 101.221 | 1.00 | 22.75 | W | O |
| ATOM | 2611 | O | HOH W | 36 | 70.118 | 14.389 | 108.156 | 1.00 | 55.54 | W | O |
| ATOM | 2612 | O | HOH W | 37 | 60.170 | 4.215 | 113.980 | 1.00 | 40.21 | W | O |
| ATOM | 2613 | O | HOH W | 38 | 50.540 | 23.541 | 133.215 | 1.00 | 2.46 | W | O |
| ATOM | 2614 | O | HOH W | 39 | 48.871 | 4.037 | 141.580 | 1.00 | 13.61 | W | O |
| ATOM | 2615 | O | HOH W | 40 | 59.625 | 29.594 | 154.497 | 1.00 | 17.76 | W | O |
| ATOM | 2616 | O | HOH W | 41 | 58.300 | 23.513 | 135.522 | 1.00 | 22.04 | W | O |
| ATOM | 2617 | O | HOH W | 42 | 64.850 | 17.232 | 147.313 | 1.00 | 21.84 | W | O |
| ATOM | 2618 | O | HOH W | 43 | 49.326 | 3.982 | 150.548 | 1.00 | 24.48 | W | O |
| ATOM | 2619 | O | HOH W | 45 | 63.774 | 19.112 | 148.870 | 1.00 | 12.98 | W | O |
| ATOM | 2620 | O | HOH W | 46 | 55.863 | 15.629 | 163.760 | 1.00 | 28.09 | W | O |
| ATOM | 2621 | O | HOH W | 47 | 53.389 | 24.883 | 133.848 | 1.00 | 20.87 | W | O |
| ATOM | 2622 | O | HOH W | 48 | 47.215 | 9.463 | 133.451 | 1.00 | 38.63 | W | O |
| ATOM | 2623 | O | HOH W | 49 | 54.340 | 11.753 | 153.786 | 1.00 | 25.99 | W | O |
| ATOM | 2624 | O | HOH W | 50 | 61.472 | 23.563 | 144.309 | 1.00 | 18.90 | W | O |
| ATOM | 2625 | O | HOH W | 51 | 63.912 | 24.339 | 144.017 | 1.00 | 19.40 | W | O |
| ATOM | 2626 | O | HOH W | 52 | 58.245 | 29.246 | 145.388 | 1.00 | 26.64 | W | O |
| ATOM | 2627 | O | HOH W | 53 | 63.745 | 27.746 | 148.959 | 1.00 | 20.88 | W | O |
| ATOM | 2628 | O | HOH W | 54 | 51.102 | 26.318 | 167.875 | 1.00 | 28.44 | W | O |
| ATOM | 2629 | O | HOH W | 55 | 52.106 | 25.451 | 162.757 | 1.00 | 26.05 | W | O |
| ATOM | 2630 | O | HOH W | 56 | 62.401 | 12.111 | 136.745 | 1.00 | 22.15 | W | O |
| ATOM | 2631 | O | HOH W | 57 | 45.079 | 38.240 | 157.288 | 1.00 | 46.25 | W | O |
| ATOM | 2632 | O | HOH W | 58 | 64.533 | 23.652 | 138.613 | 1.00 | 23.45 | W | O |
| ATOM | 2633 | O | HOH W | 59 | 32.586 | 28.259 | 147.755 | 1.00 | 53.44 | W | O |
| ATOM | 2634 | O | HOH W | 60 | 60.965 | 22.491 | 159.169 | 1.00 | 36.88 | W | O |
| ATOM | 2635 | O | HOH W | 61 | 56.540 | 16.675 | 157.926 | 1.00 | 22.67 | W | O |
| ATOM | 2636 | O | HOH W | 62 | 49.724 | 1.457 | 152.163 | 1.00 | 31.25 | W | O |
| ATOM | 2637 | O | HOH W | 63 | 66.979 | 17.779 | 135.834 | 1.00 | 30.64 | W | O |
| ATOM | 2638 | O | HOH W | 64 | 53.679 | 7.411 | 152.678 | 1.00 | 24.59 | W | O |
| ATOM | 2639 | O | HOH W | 65 | 55.862 | 13.242 | 157.045 | 1.00 | 22.19 | W | O |
| ATOM | 2640 | O | HOH W | 66 | 60.317 | 27.514 | 146.861 | 1.00 | 50.28 | W | O |
| ATOM | 2641 | O | HOH W | 67 | 48.564 | 34.049 | 142.813 | 1.00 | 37.03 | W | O |
| ATOM | 2642 | O | HOH W | 68 | 47.734 | 34.009 | 159.841 | 1.00 | 57.14 | W | O |
| ATOM | 2643 | O | HOH W | 69 | 56.949 | 31.429 | 147.112 | 1.00 | 30.39 | W | O |
| ATOM | 2644 | O | HOH W | 70 | 47.008 | 25.477 | 120.702 | 1.00 | 23.76 | W | O |
| ATOM | 2645 | O | HOH W | 71 | 53.453 | 29.741 | 136.903 | 1.00 | 25.90 | W | O |
| ATOM | 2646 | O | HOH W | 72 | 54.023 | 18.711 | 159.706 | 1.00 | 18.27 | W | O |
| ATOM | 2647 | O | HOH W | 73 | 54.530 | 9.056 | 154.663 | 1.00 | 34.56 | W | O |
| ATOM | 2648 | O | HOH W | 74 | 66.399 | 14.925 | 149.889 | 1.00 | 36.21 | W | O |
| ATOM | 2649 | O | HOH W | 75 | 56.078 | 0.717 | 148.547 | 1.00 | 47.62 | W | O |
| ATOM | 2650 | O | HOH W | 76 | 55.037 | 3.104 | 145.929 | 1.00 | 22.70 | W | O |
| ATOM | 2651 | O | HOH W | 77 | 57.979 | 5.727 | 117.915 | 1.00 | 65.95 | W | O |
| ATOM | 2652 | O | HOH W | 78 | 38.602 | 9.525 | 136.215 | 1.00 | 34.41 | W | O |
| ATOM | 2653 | O | HOH W | 79 | 56.644 | 33.927 | 107.571 | 1.00 | 25.40 | W | O |
| ATOM | 2654 | O | HOH W | 80 | 56.901 | 12.608 | 98.500 | 1.00 | 45.22 | W | O |
| ATOM | 2655 | O | HOH W | 81 | 66.159 | 25.939 | 129.659 | 1.00 | 45.73 | W | O |
| ATOM | 2656 | O | HOH W | 82 | 52.849 | 33.406 | 118.492 | 1.00 | 33.45 | W | O |
| ATOM | 2657 | O | HOH W | 83 | 40.064 | 12.132 | 132.917 | 1.00 | 45.44 | W | O |
| ATOM | 2658 | O | HOH W | 84 | 61.356 | 7.072 | 144.041 | 1.00 | 52.29 | W | O |
| ATOM | 2659 | O | HOH W | 85 | 59.332 | 33.793 | 126.661 | 1.00 | 37.65 | W | O |
| ATOM | 2660 | O | HOH W | 86 | 76.248 | 14.005 | 117.780 | 1.00 | 38.05 | W | O |
| ATOM | 2661 | O | HOH W | 87 | 63.920 | 4.959 | 114.145 | 1.00 | 28.43 | W | O |
| ATOM | 2662 | O | HOH W | 88 | 41.594 | 23.567 | 106.694 | 1.00 | 35.95 | W | O |
| ATOM | 2663 | O | HOH W | 89 | 43.961 | 20.340 | 105.825 | 1.00 | 34.92 | W | O |
| ATOM | 2664 | O | HOH W | 90 | 73.852 | 13.594 | 117.386 | 1.00 | 26.01 | W | O |
| ATOM | 2665 | O | HOH W | 91 | 59.941 | 14.152 | 100.157 | 1.00 | 27.50 | W | O |
| ATOM | 2666 | O | HOH W | 92 | 59.242 | 23.420 | 132.602 | 1.00 | 16.67 | W | O |
| ATOM | 2667 | O | HOH W | 93 | 58.837 | 25.317 | 128.952 | 1.00 | 21.05 | W | O |
| ATOM | 2668 | O | HOH W | 94 | 51.613 | 25.829 | 131.530 | 1.00 | 40.97 | W | O |
| ATOM | 2669 | O | HOH W | 95 | 64.674 | 28.352 | 131.418 | 1.00 | 31.06 | W | O |
| ATOM | 2670 | O | HOH W | 96 | 60.935 | 29.269 | 136.385 | 1.00 | 24.27 | W | O |
| ATOM | 2671 | O | HOH W | 97 | 54.832 | 34.703 | 109.520 | 1.00 | 17.56 | W | O |
| ATOM | 2672 | O | HOH W | 98 | 58.791 | 32.739 | 117.544 | 1.00 | 24.04 | W | O |
| ATOM | 2673 | O | HOH W | 99 | 56.442 | 35.249 | 118.352 | 1.00 | 28.89 | W | O |
| ATOM | 2674 | O | HOH W | 100 | 46.526 | 37.510 | 114.770 | 1.00 | 35.71 | W | O |
| ATOM | 2675 | O | HOH W | 101 | 54.579 | 35.72 | 1151.192 | 1.00 | 51.01 | W | O |
| ATOM | 2676 | O | HOH W | 102 | 58.105 | 32.269 | 149.264 | 1.00 | 23.65 | W | O |
| ATOM | 2677 | O | HOH W | 103 | 65.313 | 13.049 | 157.092 | 1.00 | 26.96 | W | O |
| ATOM | 2678 | O | HOH W | 104 | 66.201 | 11.386 | 155.250 | 1.00 | 48.24 | W | O |
| ATOM | 2679 | O | HOH W | 105 | 53.996 | 0.840 | 145.922 | 1.00 | 34.76 | W | O |
| ATOM | 2680 | O | HOH W | 106 | 46.202 | 0.833 | 136.125 | 1.00 | 47.74 | W | O |
| ATOM | 2681 | O | HOH W | 107 | 56.086 | 6.461 | 135.385 | 1.00 | 47.82 | W | O |
| ATOM | 2682 | O | HOH W | 108 | 51.570 | 16.588 | 127.129 | 1.00 | 18.01 | W | O |
| ATOM | 2683 | O | HOH W | 109 | 46.668 | 19.140 | 128.889 | 1.00 | 23.88 | W | O |
| ATOM | 2684 | O | HOH W | 110 | 45.451 | 12.416 | 128.248 | 1.00 | 32.22 | W | O |
| ATOM | 2685 | O | HOH W | 111 | 53.514 | 30.754 | 139.864 | 1.00 | 31.81 | W | O |
| ATOM | 2686 | O | HOH W | 112 | 52.273 | 39.562 | 147.763 | 1.00 | 51.12 | W | O |
| ATOM | 2687 | O | HOH W | 113 | 52.591 | 10.981 | 161.536 | 1.00 | 34.69 | W | O |
| ATOM | 2688 | O | HOH W | 114 | 50.264 | 24.639 | 126.956 | 1.00 | 19.43 | W | O |
| ATOM | 2689 | O | HOH W | 115 | 45.122 | 15.259 | 128.545 | 1.00 | 36.82 | W | O |
| ATOM | 2690 | O | HOH W | 116 | 45.881 | 15.698 | 122.389 | 1.00 | 39.53 | W | O |
| ATOM | 2691 | O | HOH W | 117 | 45.448 | 17.802 | 130.938 | 1.00 | 37.87 | W | O |
| ATOM | 2692 | O | HOH W | 118 | 58.454 | 6.855 | 127.071 | 1.00 | 27.25 | W | O |
| ATOM | 2693 | O | HOH W | 119 | 62.131 | 28.235 | 104.517 | 1.00 | 40.71 | W | O |
| ATOM | 2694 | O | HOH W | 120 | 66.292 | 32.682 | 114.288 | 1.00 | 25.89 | W | O |
| ATOM | 2695 | O | HOH W | 121 | 65.759 | 26.751 | 110.354 | 1.00 | 19.28 | W | O |
| END | | | | | | | | | | | |

TABLE 3
Three dimensional atomic coordinates of the MMP9 mutant (E402Q
construct in complex with the reverse hydroxamate compound of Formula I.

REMARK coordinates from restrained individual B-factor refinement.
REMARK refinement resolution: 100 -2.3 A
REMARK starting r = 0.2096 free_r = 0.2397
REMARK final r = 0.2074 free_r = 0.2408
REMARK B rmsd for bonded mainchain atoms = 1.707 target = 1.5
REMARK B rmsd for bonded sidechain atoms = 2.673 target = 2.0
REMARK B rmsd for angle mainchain atoms = 2.714 target = 2.0
REMARK B rmsd for angle sidechain atoms = 3.856 target = 2.5
REMARK wa = 2.19316
REMARK rweight = 5.978512E−02
REMARK target = mlf steps = 30
REMARK sg = P4(1)2(1)2 a = 56.005 b = 56.005 c = 262.655 alpha = 90 beta = 90 gamma = 9
REMARK parameter file 1: MSI_CNX_TOPPAR: protein_rep.param
REMARK parameter file 2: MSI_CNX_TOPPAR: ion.param
REMARK parameter file 3: MSI_CNX_TOPPAR: water_rep.param
REMARK parameter file 4: fra.par
REMARK molecular structure file: mmp9.mtf
REMARK input coordinates: anneal_restrain_1.pdb
REMARK reflection file = mmp9_rf.hkl
REMARK ncs = restrain ncs file = ncs_restrain.def
REMARK B-correction resolution: 6.0-2.3
REMARK initial B-factor correction applied to fobs:
REMARK B11 = −4.644 B22 = −4.644 B33 = 9.288
REMARK B12 = 0.000 B13 = 0.000 B23 = 0.000
REMARK B-factor correction applied to coordinate array B: 0.533
REMARK bulk solvent: (Mask) density level 0.372729 e/A\hat{ }3, B-factor 48.8354 A\hat{ 2}
REMARK reflections with |Fobs|/sigma_F < 0.0 rejected
REMARK reflections with |Fobs| > 1000 * rms(Fobs) rejected
REMARK theoretical total number of refl. in resol. range: 19670 (100.0%)
REMARK number of unobserved reflections (no entry or |F| =0): 2295 (11.7%)
REMARK number of reflections rejected: 0(0.0%)
REMARK total number of reflections used: 17375 (88.3%)
REMARK number of reflections in working set: 16691 (84.9%)
REMARK number of reflections in test set: 684 (3.5%)
REMARK FILENAME = ″bindividual7.pdb″
REMARK DATE: Nov. 30, 2000 13:59:53 created by user: sian
REMARK Written by CNX VBRSION: 2000
CRYST1 56.005 56.005 262.655 90.00 90.00 90.00 P 41 21 2
SCALE1 0.017856 0.000000 0.000000 0.00000
SCALE2 0.000000 0.017856 0.000000 0.00000
SCALE3 0.000000 0.000000 0.003807 0.00000

| | | | | | | | | | | | | |
|---|---|---|---|---|---|---|---|---|---|---|---|---|
| ATOM | 1 | CB | PHE | A | 110 | 68.144 | 27.293 | 107.042 | 1.00 | 76.67 | A | C |
| ATOM | 2 | CG | FHE | A | 110 | 69.059 | 26.231 | 107.577 | 1.00 | 76.83 | A | C |
| ATOM | 3 | CD1 | PHE | A | 110 | 70.433 | 26.309 | 107.372 | 1.00 | 76.63 | A | C |
| ATOM | 4 | CD2 | PHE | A | 110 | 68.547 | 25.145 | 108.276 | 1.00 | 76.62 | A | C |
| ATOM | S | CE1 | PHE | A | 110 | 71.282 | 25.322 | 107.853 | 1.00 | 75.40 | A | C |
| ATOM | 6 | CE2 | PHE | A | 110 | 69.390 | 24.152 | 108.763 | 1.00 | 76.11 | A | C |
| ATOM | 7 | CZ | PHE | A | 110 | 70.760 | 24.241 | 108.550 | 1.00 | 75.74 | A | C |
| ATOM | 8 | C | PHE | A | 110 | 67.022 | 28.261 | 105.029 | 1.00 | 76.18 | A | C |
| ATOM | 9 | O | PHE | A | 110 | 65.965 | 28.280 | 105.664 | 1.00 | 76.86 | A | O |
| ATOM | 10 | N | PHE | A | 110 | 69.510 | 28.084 | 105.133 | 1.00 | 76.75 | A | N |
| ATOM | 11 | CA | PHE | A | 110 | 68.217 | 27.455 | 105.522 | 1.00 | 76.45 | A | C |
| ATOM | 12 | N | GLU | A | 111 | 67.204 | 28.928 | 103.893 | 1.00 | 74.63 | A | N |
| ATOM | 13 | CA | GLU | A | 111 | 66.148 | 29.724 | 103.279 | 1.00 | 73.01 | A | C |
| ATOM | 14 | CB | GLU | A | 111 | 66.490 | 31.215 | 103.335 | 1.00 | 75.82 | A | C |
| ATOM | 15 | CO | GLU | A | 111 | 66.819 | 31.745 | 104.726 | 1.00 | 80.41 | A | C |
| ATOM | 16 | CD | GLU | A | 111 | 65.689 | 31.554 | 105.725 | 1.00 | 83.58 | A | C |
| ATOM | 17 | OE1 | GLU | A | 111 | 64.561 | 32.022 | 105.452 | 1.00 | 85.28 | A | O |
| ATOM | 18 | OE2 | GLU | A | 111 | 65.934 | 30.941 | 106.789 | 1.00 | 84.02 | A | O |
| ATOM | 19 | C | GLU | A | 111 | 66.025 | 29.282 | 101.826 | 1.00 | 69.95 | A | C |
| ATOM | 20 | O | GLU | A | 111 | 67.025 | 29.180 | 101.111 | 1.00 | 70.43 | A | O |
| ATOM | 21 | N | GLY | A | 112 | 64.801 | 29.009 | 101.394 | 1.00 | 66.03 | A | N |
| ATOM | 22 | CA | GLY | A | 112 | 64.587 | 28.577 | 100.027 | 1.00 | 60.34 | A | C |
| ATOM | 23 | C | GLY | A | 112 | 63.299 | 29.143 | 99.479 | 1.00 | 56.89 | A | C |
| ATOM | 24 | O | GLY | A | 112 | 62.515 | 29.744 | 100.213 | 1.00 | 57.97 | A | O |
| ATOM | 25 | N | ASP | A | 113 | 63.077 | 28.955 | 98.185 | 1.00 | 53.94 | A | N |
| ATOM | 26 | CA | ASP | A | 113 | 61.866 | 29.459 | 97.553 | 1.00 | 50.40 | A | C |
| ATOM | 27 | CB | ASP | A | 113 | 62.209 | 30.556 | 96.543 | 1.00 | 52.58 | A | C |
| ATOM | 28 | CG | ASP | A | 113 | 62.790 | 31.792 | 97.202 | 1.00 | 53.44 | A | C |
| ATOM | 29 | OD1 | ASP | A | 113 | 62.092 | 32.405 | 98.039 | 1.00 | 53.76 | A | O |
| ATOM | 30 | OD2 | ASP | A | 113 | 63.944 | 32.146 | 96.885 | 1.00 | 53.72 | A | O |
| ATOM | 31 | C | ASP | A | 113 | 61.098 | 28.345 | 96.867 | 1.00 | 46.20 | A | C |
| ATOM | 32 | O | ASP | A | 113 | 60.589 | 28.519 | 95.763 | 1.00 | 46.06 | A | O |
| ATOM | 33 | N | LEU | A | 114 | 61.024 | 27.198 | 97.535 | 1.00 | 40.51 | A | N |
| ATOM | 34 | CA | LEU | A | 114 | 60.309 | 26.043 | 97.018 | 1.00 | 35.29 | A | C |
| ATOM | 35 | CB | LEU | A | 114 | 61.265 | 24.870 | 96.812 | 1.00 | 35.18 | A | C |
| ATOM | 36 | CG | LEU | A | 114 | 62.424 | 25.022 | 95.829 | 1.00 | 35.74 | A | C |
| ATOM | 37 | CD1 | LEU | A | 114 | 63.155 | 23.689 | 95.726 | 1.00 | 34.64 | A | C |
| ATOM | 38 | CD2 | LEU | A | 114 | 61.906 | 25.455 | 94.463 | 1.00 | 31.83 | A | C |
| ATOM | 39 | C | LEU | A | 114 | 59.229 | 25.624 | 98.003 | 1.00 | 33.60 | A | C |
| ATOM | 40 | O | LEU | A | 114 | 59.363 | 25.842 | 99.205 | 1.00 | 33.68 | A | O |
| ATOM | 41 | N | LYS | A | 115 | 58.151 | 25.040 | 97.489 | 1.00 | 30.09 | A | N |
| ATOM | 42 | CA | LYS | A | 115 | 57.066 | 24.556 | 98.331 | 1.00 | 28.79 | A | C |
| ATOM | 43 | CB | LYS | A | 115 | 55.782 | 25.350 | 98.091 | 1.00 | 27.23 | A | C |
| ATOM | 44 | CG | LYS | A | 115 | 55.136 | 25.113 | 96.739 | 1.00 | 30.27 | A | C |
| ATOM | 45 | CD | LYS | A | 115 | 53.726 | 25.671 | 96.711 | 1.00 | 31.99 | A | C |
| ATOM | 46 | CE | LYS | A | 115 | 53.015 | 25.309 | 95.420 | 1.00 | 33.32 | A | C |
| ATOM | 47 | NZ | LYS | A | 115 | 51.602 | 25.772 | 95.434 | 1.00 | 31.74 | A | N |
| ATOM | 48 | C | LYS | A | 115 | 56.854 | 23.099 | 97.946 | 1.00 | 27.74 | A | C |
| ATOM | 49 | O | LYS | A | 115 | 57.490 | 22.610 | 97.017 | 1.00 | 26.52 | A | O |
| ATOM | 50 | N | TRP | A | 116 | 55.984 | 22.398 | 98.660 | 1.00 | 26.86 | A | N |
| ATOM | 51 | CA | TRP | A | 116 | 55.725 | 21.005 | 98.327 | 1.00 | 26.50 | A | C |
| ATOM | 52 | CB | TRP | A | 116 | 55.139 | 20.242 | 99.525 | 1.00 | 24.26 | A | C |
| ATOM | 53 | CG | TRP | A | 116 | 56.071 | 20.142 | 100.708 | 1.00 | 26.96 | A | C |
| ATOM | 54 | CD2 | TRP | A | 116 | 57.243 | 19.321 | 100.810 | 1.00 | 26.47 | A | C |
| ATOM | 55 | CE2 | TRP | A | 116 | 57.806 | 19.554 | 102.087 | 1.00 | 25.23 | A | C |
| ATOM | 56 | CE3 | TRP | A | 116 | 57.873 | 18.414 | 99.946 | 1.00 | 25.47 | A | C |
| ATOM | 57 | CD1 | TRP | A | 116 | 55.977 | 20.821 | 101.894 | 1.00 | 23.47 | A | C |
| ATOM | 58 | NE1 | TRP | A | 116 | 57.014 | 20.472 | 102.724 | 1.00 | 22.33 | A | N |
| ATOM | 59 | CZ2 | TRP | A | 116 | 58.972 | 18.910 | 102.521 | 1.00 | 24.08 | A | C |
| ATOM | 60 | CZ3 | TRP | A | 116 | 59.037 | 17.774 | 100.378 | 1.00 | 24.64 | A | C |
| ATOM | 61 | CH2 | TRP | A | 116 | 59.572 | 18.027 | 101.654 | 1.00 | 25.34 | A | C |
| ATOM | 62 | C | TRP | A | 116 | 54.723 | 20.991 | 97.184 | 1.00 | 27.50 | A | C |
| ATOM | 63 | O | TRP | A | 116 | 53.828 | 21.836 | 97.125 | 1.00 | 25.79 | A | O |
| ATOM | 64 | N | HIS | A | 117 | 54.877 | 20.039 | 96.271 | 1.00 | 27.87 | A | N |
| ATOM | 65 | CA | HIS | A | 117 | 53.950 | 19.932 | 95.158 | 1.00 | 29.48 | A | C |
| ATOM | 66 | CB | HIS | A | 117 | 54.692 | 19.528 | 93.885 | 1.00 | 25.71 | A | C |
| ATOM | 67 | CG | HIS | A | 117 | 55.384 | 20.674 | 93.214 | 1.00 | 27.00 | A | C |
| ATOM | 68 | CD2 | HIS | A | 117 | 55.394 | 21.998 | 93.501 | 1.00 | 27.93 | A | C |
| ATOM | 69 | ND1 | HIS | A | 117 | 56.184 | 20.520 | 92.103 | 1.00 | 28.15 | A | N |
| ATOM | 70 | CE1 | HIS | A | 117 | 56.659 | 21.697 | 91.736 | 1.00 | 25.56 | A | C |
| ATOM | 71 | NE2 | HIS | A | 117 | 56.195 | 22.611 | 92.568 | 1.00 | 25.76 | A | N |
| ATOM | 72 | C | HIS | A | 117 | 52.794 | 18.982 | 95.465 | 1.00 | 31.31 | A | C |
| ATOM | 73 | O | HIS | A | 117 | 51.849 | 18.873 | 94.687 | 1.00 | 36.62 | A | O |
| ATOM | 74 | N | HIS | A | 118 | 52.868 | 18.292 | 96.600 | 1.00 | 28.91 | A | N |
| ATOM | 75 | CA | HIS | A | 118 | 51.782 | 17.412 | 97.007 | 1.00 | 27.75 | A | C |
| ATOM | 76 | CB | HIS | A | 118 | 52.257 | 15.966 | 97.160 | 1.00 | 26.46 | A | C |
| ATOM | 77 | CG | HIS | A | 118 | 53.454 | 15.800 | 98.041 | 1.00 | 28.06 | A | C |
| ATOM | 78 | CD2 | HIS | A | 118 | 53.587 | 15.230 | 99.262 | 1.00 | 26.27 | A | C |
| ATOM | 79 | ND1 | HIS | A | 118 | 54.715 | 16.213 | 97.672 | 1.00 | 27.31 | A | N |
| ATOM | 80 | CE1 | HIS | A | 118 | 55.575 | 15.901 | 98.625 | 1.00 | 25.21 | A | C |
| ATOM | 81 | NE2 | HIS | A | 118 | 54.916 | 15.304 | 99.600 | 1.00 | 25.44 | A | N |
| ATOM | 82 | C | HIS | A | 118 | 51.232 | 17.949 | 98.322 | 1.00 | 28.02 | A | C |
| ATOM | 83 | O | HIS | A | 118 | 51.906 | 18.717 | 99.006 | 1.00 | 30.48 | A | O |
| ATOM | 84 | N | HIS | A | 119 | 50.008 | 17.566 | 98.670 | 1.00 | 28.21 | A | N |
| ATOM | 85 | CA | HIS | A | 119 | 49.394 | 18.049 | 99.900 | 1.00 | 28.31 | A | C |
| ATOM | 86 | CB | HIS | A | 119 | 48.001 | 18.597 | 99.607 | 1.00 | 27.88 | A | C |
| ATOM | 87 | CG | HIS | A | 119 | 48.015 | 19.881 | 98.843 | 1.00 | 30.03 | A | C |
| ATOM | 88 | CD2 | HIS | A | 119 | 49.045 | 20.642 | 98.402 | 1.00 | 28.56 | A | C |
| ATOM | 89 | ND1 | HIS | A | 119 | 46.862 | 20.530 | 98.452 | 1.00 | 32.34 | A | N |
| ATOM | 90 | CE1 | HIS | A | 119 | 47.182 | 21.636 | 97.804 | 1.00 | 30.91 | A | C |
| ATOM | 91 | NE2 | HIS | A | 119 | 48.500 | 21.728 | 97.760 | 1.00 | 30.11 | A | N |
| ATOM | 92 | C | HIS | A | 119 | 49.318 | 17.040 | 101.034 | 1.00 | 28.11 | A | C |
| ATOM | 93 | O | HIS | A | 119 | 49.120 | 17.424 | 102.184 | 1.00 | 28.79 | A | O |
| ATOM | 94 | N | ASN | A | 120 | 49.447 | 15.757 | 100.715 | 1.00 | 28.28 | A | N |
| ATOM | 95 | CA | ASN | A | 120 | 49.418 | 14.724 | 101.742 | 1.00 | 30.67 | A | C |
| ATOM | 96 | CB | ASN | A | 120 | 48.753 | 13.442 | 101.206 | 1.00 | 35.59 | A | C |
| ATOM | 97 | CG | ASN | A | 120 | 49.339 | 12.970 | 99.874 | 1.00 | 40.97 | A | C |
| ATOM | 98 | OD1 | ASN | A | 120 | 49.628 | 13.776 | 98.982 | 1.00 | 42.91 | A | O |
| ATOM | 99 | ND2 | ASN | A | 120 | 49.494 | 11.652 | 99.728 | 1.00 | 42.68 | A | N |
| ATOM | 100 | C | ASN | A | 120 | 50.873 | 14.489 | 102.141 | 1.00 | 29.30 | A | C |
| ATOM | 101 | O | ASN | A | 120 | 51.564 | 13.634 | 101.585 | 1.00 | 29.67 | A | O |
| ATOM | 102 | N | ILE | A | 121 | 51.330 | 15.287 | 103.103 | 1.00 | 26.98 | A | N |
| ATOM | 103 | CA | ILE | A | 121 | 52.710 | 15.233 | 103.577 | 1.00 | 26.04 | A | C |
| ATOM | 104 | CB | ILE | A | 121 | 53.138 | 16.617 | 104.109 | 1.00 | 26.73 | A | C |
| ATOM | 105 | CG2 | ILE | A | 121 | 54.574 | 16.568 | 104.626 | 1.00 | 26.58 | A | C |
| ATOM | 106 | CG1 | ILE | A | 121 | 52.997 | 17.650 | 102.987 | 1.00 | 29.00 | A | C |
| ATOM | 107 | CD1 | ILE | A | 121 | 53.451 | 19.037 | 103.357 | 1.00 | 32.81 | A | C |
| ATOM | 108 | C | ILE | A | 121 | 52.945 | 14.179 | 104.650 | 1.00 | 23.95 | A | C |
| ATOM | 109 | O | ILE | A | 121 | 52.081 | 13.928 | 105.484 | 1.00 | 24.37 | A | O |
| ATOM | 110 | N | THR | A | 122 | 54.119 | 13.560 | 104.621 | 1.00 | 20.08 | A | N |
| ATOM | 111 | CA | THR | A | 122 | 54.441 | 12.534 | 105.598 | 1.00 | 21.17 | A | C |
| ATOM | 112 | CB | THR | A | 122 | 54.763 | 11.172 | 104.914 | 1.00 | 22.42 | A | C |
| ATOM | 113 | OG1 | THR | A | 122 | 55.948 | 11.294 | 104.115 | 1.00 | 20.53 | A | O |
| ATOM | 114 | CG2 | THR | A | 122 | 53.592 | 19.722 | 104.042 | 1.00 | 21.00 | A | C |
| ATOM | 115 | C | THR | A | 122 | 55.624 | 12.925 | 106.481 | 1.00 | 20.68 | A | C |
| ATOM | 116 | O | THR | A | 122 | 56.502 | 13.706 | 106.081 | 1.00 | 18.28 | A | O |
| ATOM | 117 | N | TYR | A | 123 | 55.640 | 12.382 | 107.693 | 1.00 | 18.69 | A | N |
| ATOM | 118 | CA | TYR | A | 123 | 56.728 | 12.671 | 108.612 | 1.00 | 17.02 | A | C |
| ATOM | 119 | CB | TYR | A | 123 | 56.374 | 13.828 | 109.561 | 1.00 | 12.64 | A | C |
| ATOM | 120 | CG | TYR | A | 123 | 55.291 | 13.533 | 110.579 | 1.00 | 14.99 | A | C |
| ATOM | 121 | CD1 | TYR | A | 123 | 53.949 | 13.737 | 110.277 | 1.00 | 15.83 | A | C |
| ATOM | 122 | CE1 | TYR | A | 123 | 52.953 | 13.517 | 111.228 | 1.00 | 14.44 | A | C |
| ATOM | 123 | CD2 | TYR | A | 123 | 55.613 | 13.088 | 111.867 | 1.00 | 15.15 | A | C |
| ATOM | 124 | CE2 | TYR | A | 123 | 54.623 | 12.866 | 112.821 | 1.00 | 13.15 | A | C |
| ATOM | 125 | CZ | TYR | A | 123 | 53.296 | 13.087 | 112.495 | 1.00 | 14.54 | A | C |
| ATOM | 126 | OH | TYR | A | 123 | 52.305 | 12.912 | 113.439 | 1.00 | 17.15 | A | O |
| ATOM | 127 | C | TYR | A | 123 | 57.109 | 11.446 | 109.419 | 1.00 | 15.63 | A | C |
| ATOM | 128 | O | TYR | A | 123 | 56.277 | 10.594 | 109.735 | 1.00 | 14.88 | A | O |
| ATOM | 129 | N | TRP | A | 124 | 58.387 | 11.380 | 109.745 | 1.00 | 14.30 | A | N |
| ATOM | 130 | CA | TRP | A | 124 | 58.933 | 10.288 | 110.504 | 1.00 | 15.16 | A | C |
| ATOM | 131 | CB | TRP | A | 124 | 59.876 | 9.486 | 109.603 | 1.00 | 17.22 | A | C |
| ATOM | 132 | CG | TRP | A | 124 | 60.649 | 8.390 | 110.274 | 1.00 | 14.19 | A | C |
| ATOM | 133 | CD2 | TRP | A | 124 | 62.005 | 8.019 | 110.004 | 1.00 | 17.22 | A | C |
| ATOM | 134 | CE2 | TRP | A | 124 | 62.299 | 6.892 | 110.807 | 1.00 | 18.18 | A | C |
| ATOM | 135 | CE3 | TRP | A | 124 | 63.002 | 8.525 | 109.157 | 1.00 | 17.93 | A | C |
| ATOM | 136 | CD1 | TRP | A | 124 | 60.186 | 7.506 | 111.204 | 1.00 | 15.79 | A | C |
| ATOM | 137 | NE1 | TRP | A | 124 | 61.172 | 6.599 | 111.530 | 1.00 | 17.60 | A | N |
| ATOM | 138 | CZ2 | TRP | A | 124 | 63.548 | 6.262 | 110.788 | 1.00 | 17.69 | A | C |
| ATOM | 139 | CZ3 | TRP | A | 124 | 64.244 | 7.899 | 109.139 | 1.00 | 22.22 | A | C |
| ATOM | 140 | CH2 | TRP | A | 124 | 64.505 | 6.776 | 109.951 | 1.00 | 19.94 | A | C |
| ATOM | 141 | C | TRP | A | 124 | 59.679 | 10.821 | 111.720 | 1.00 | 15.01 | A | C |
| ATOM | 142 | O | TRP | A | 124 | 60.575 | 11.645 | 111.590 | 1.00 | 15.43 | A | O |
| ATOM | 143 | N | ILE | A | 125 | 59.283 | 10.365 | 112.903 | 1.00 | 13.81 | A | N |
| ATOM | 144 | CA | ILB | A | 125 | 59.960 | 10.754 | 114.127 | 1.00 | 14.20 | A | C |
| ATOM | 145 | CB | ILE | A | 125 | 59.009 | 10.697 | 115.341 | 1.00 | 14.44 | A | C |
| ATOM | 146 | CG2 | ILE | A | 125 | 59.758 | 11.087 | 116.609 | 1.00 | 10.86 | A | C |
| ATOM | 147 | CG1 | ILB | A | 125 | 57.833 | 11.649 | 115.110 | 1.00 | 14.53 | A | C |
| ATOM | 148 | CD1 | ILE | A | 125 | 56.721 | 11.500 | 116.106 | 1.00 | 16.01 | A | C |
| ATOM | 149 | C | ILE | A | 125 | 61.038 | 9.685 | 114.249 | 1.00 | 14.73 | A | C |
| ATOM | 150 | O | ILE | A | 125 | 60.763 | 8.564 | 114.660 | 1.00 | 17.21 | A | O |
| ATOM | 151 | N | GLN | A | 126 | 62.259 | 10.036 | 113.863 | 1.00 | 15.17 | A | N |
| ATOM | 152 | CA | GLN | A | 126 | 63.374 | 9.102 | 113.877 | 1.00 | 17.19 | A | C |
| ATOM | 153 | CB | GLN | A | 126 | 64.540 | 9.679 | 113.065 | 1.00 | 15.71 | A | C |
| ATOM | 154 | CG | GLN | A | 126 | 65.810 | 8.849 | 113.134 | 1.00 | 19.08 | A | C |
| ATOM | 155 | CD | GLN | A | 126 | 67.006 | 9.537 | 112.492 | 1.00 | 21.76 | A | C |
| ATOM | 156 | OB1 | GLN | A | 126 | 68.141 | 9.434 | 112.978 | 1.00 | 21.48 | A | O |
| ATOM | 157 | NE2 | GLN | A | 126 | 66.762 | 10.235 | 111.395 | 1.00 | 21.97 | A | N |
| ATOM | 158 | C | GLN | A | 126 | 63.858 | 8.702 | 115.270 | 1.00 | 18.20 | A | C |
| ATOM | 159 | O | GLN | A | 126 | 64.062 | 7.519 | 115.536 | 1.00 | 18.29 | A | O |
| ATOM | 160 | N | ASN | A | 127 | 64.066 | 9.681 | 116.145 | 1.00 | 19.88 | A | N |
| ATOM | 161 | CA | ASN | A | 127 | 64.515 | 9.397 | 117.504 | 1.00 | 18.22 | A | C |
| ATOM | 162 | CB | ASN | A | 127 | 66.942 | 9.423 | 117.594 | 1.00 | 19.01 | A | C |
| ATOM | 163 | CG | ASN | A | 127 | 66.631 | 10.770 | 117.242 | 1.00 | 21.39 | A | C |
| ATOM | 164 | OD1 | ASN | A | 127 | 65.957 | 11.798 | 117.310 | 1.00 | 25.90 | A | O |
| ATOM | 165 | ND2 | ASN | A | 127 | 67.909 | 10.774 | 116.880 | 1.00 | 20.17 | A | N |
| ATOM | 166 | C | ASN | A | 127 | 63.900 | 10.386 | 118.483 | 1.00 | 19.18 | A | C |
| ATOM | 167 | O | ASN | A | 127 | 63.238 | 11.341 | 118.068 | 1.00 | 18.72 | A | O |
| ATOM | 168 | N | TYR | A | 128 | 64.128 | 10.163 | 119.779 | 1.00 | 17.90 | A | N |
| ATOM | 169 | CA | TYR | A | 128 | 63.539 | 11.006 | 120.822 | 1.00 | 15.61 | A | C |
| ATOM | 170 | CB | TYR | A | 128 | 62.592 | 10.166 | 121.693 | 1.00 | 15.35 | A | C |
| ATOM | 171 | CG | TYR | A | 128 | 61.389 | 9.623 | 120.957 | 1.00 | 14.14 | A | C |
| ATOM | 172 | CD1 | TYR | A | 128 | 61.476 | 8.467 | 120.178 | 1.00 | 12.53 | A | C |
| ATOM | 173 | CE1 | TYR | A | 128 | 60.374 | 8.000 | 119.469 | 1.00 | 12.28 | A | C |
| ATOM | 174 | CD2 | TYR | A | 128 | 60.172 | 10.293 | 121.008 | 1.00 | 12.14 | A | C |
| ATOM | 175 | CE2 | TYR | A | 128 | 59.078 | 9.839 | 120.306 | 1.00 | 14.27 | A | C |
| ATOM | 176 | CZ | TYR | A | 128 | 59.182 | 8.698 | 119.539 | 1.00 | 13.46 | A | C |
| ATOM | 177 | OH | TYR | A | 128 | 58.084 | 8.292 | 118.837 | 1.00 | 12.04 | A | O |
| ATOM | 178 | C | TYR | A | 128 | 64.501 | 11.716 | 121.746 | 1.00 | 14.76 | A | C |
| ATOM | 179 | O | TYR | A | 128 | 65.681 | 11.396 | 121.808 | 1.00 | 15.87 | A | O |
| ATOM | 180 | N | SER | A | 129 | 63.969 | 12.693 | 122.471 | 1.00 | 17.19 | A | N |
| ATOM | 181 | CA | SER | A | 129 | 64.742 | 13.430 | 123.460 | 1.00 | 19.66 | A | C |
| ATOM | 182 | CB | SER | A | 129 | 64.173 | 14.826 | 123.675 | 1.00 | 17.51 | A | C |
| ATOM | 183 | OG | SER | A | 129 | 64.731 | 15.394 | 124.844 | 1.00 | 16.48 | A | O |
| ATOM | 184 | C | SER | A | 129 | 64.600 | 12.640 | 124.753 | 1.00 | 22.10 | A | C |
| ATOM | 185 | O | SER | A | 129 | 63.646 | 11.882 | 124.920 | 1.00 | 21.65 | A | O |
| ATOM | 186 | N | GLU | A | 130 | 65.529 | 12.821 | 125.677 | 1.00 | 24.54 | A | N |
| ATOM | 187 | CA | GLU | A | 130 | 65.459 | 12.085 | 126.925 | 1.00 | 28.04 | A | C |
| ATOM | 188 | CB | GLU | A | 130 | 66.884 | 11.859 | 127.459 | 1.00 | 31.45 | A | C |
| ATOM | 189 | CG | GLU | A | 130 | 67.799 | 11.117 | 126.464 | 1.00 | 36.20 | A | C |
| ATOM | 190 | CD | GLU | A | 130 | 69.207 | 10.871 | 126.996 | 1.00 | 37.68 | A | C |
| ATOM | 191 | OE1 | GLU | A | 130 | 69.330 | 10.501 | 128.182 | 1.00 | 43.10 | A | O |
| ATOM | 192 | OE2 | GLU | A | 130 | 70.189 | 11.031 | 126.237 | 1.00 | 32.89 | A | O |
| ATOM | 193 | C | GLU | A | 130 | 64.587 | 12.788 | 127.971 | 1.00 | 27.09 | A | C |
| ATOM | 194 | O | GLU | A | 130 | 64.154 | 12.167 | 128.939 | 1.00 | 29.20 | A | O |
| ATOM | 195 | N | ASP | A | 131 | 64.301 | 14.069 | 127.751 | 1.00 | 23.80 | A | N |
| ATOM | 196 | CA | ASP | A | 131 | 63.519 | 14.870 | 128.693 | 1.00 | 21.55 | A | C |
| ATOM | 197 | GB | ASP | A | 131 | 63.657 | 16.355 | 128.367 | 1.00 | 20.53 | A | C |
| ATOM | 198 | CG | ASP | A | 131 | 65.084 | 16.774 | 128.149 | 1.00 | 21.96 | A | C |
| ATOM | 199 | OD1 | ASP | A | 131 | 65.993 | 16.132 | 128.718 | 1.00 | 23.79 | A | O |
| ATOM | 200 | OD2 | ASP | A | 131 | 65.291 | 17.760 | 127.417 | 1.00 | 19.14 | A | O |
| ATOM | 201 | C | ASP | A | 131 | 62.029 | 14.589 | 128.821 | 1.00 | 22.60 | A | C |
| ATOM | 202 | O | ASP | A | 131 | 61.417 | 14.946 | 129.826 | 1.00 | 22.01 | A | O |
| ATOM | 203 | N | LEU | A | 132 | 61.426 | 13.985 | 127.808 | 1.00 | 21.60 | A | N |
| ATOM | 204 | CA | LEU | A | 132 | 59.996 | 13.736 | 127.862 | 1.00 | 17.46 | A | C |
| ATOM | 205 | CB | LEU | A | 132 | 59.274 | 14.744 | 126.964 | 1.00 | 18.77 | A | C |
| ATOM | 206 | CO | LEU | A | 132 | 59.410 | 16.228 | 127.329 | 1.00 | 16.38 | A | C |
| ATOM | 207 | CD1 | LEU | A | 132 | 58.952 | 17.103 | 126.185 | 1.00 | 16.14 | A | C |
| ATOM | 208 | CD2 | LEU | A | 132 | 58.584 | 16.513 | 128.574 | 1.00 | 17.73 | A | C |
| ATOM | 209 | C | LEU | A | 132 | 59.625 | 12.322 | 127.447 | 1.00 | 17.91 | A | C |
| ATOM | 210 | O | LEU | A | 132 | 60.350 | 11.668 | 126.703 | 1.00 | 20.57 | A | O |
| ATOM | 211 | N | PRO | A | 133 | 58.493 | 11.818 | 127.952 | 1.00 | 18.54 | A | N |
| ATOM | 212 | CD | PRO | A | 133 | 57.590 | 12.397 | 128.962 | 1.00 | 16.46 | A | C |
| ATOM | 213 | CA | PRO | A | 133 | 58.070 | 10.464 | 127.588 | 1.00 | 18.30 | A | C |
| ATOM | 214 | CB | PRO | A | 133 | 56.828 | 10.250 | 128.448 | 1.00 | 16.44 | A | C |
| ATOM | 215 | CG | PRO | A | 133 | 57.029 | 11.174 | 129.599 | 1.00 | 13.96 | A | C |
| ATOM | 216 | C | PRO | A | 133 | 57.739 | 10.482 | 126.092 | 1.00 | 18.28 | A | C |
| ATOM | 217 | O | PRO | A | 133 | 57.305 | 11.511 | 125.566 | 1.00 | 15.16 | A | O |
| ATOM | 218 | N | ARG | A | 134 | 57.931 | 9.358 | 125.412 | 1.00 | 18.49 | A | N |
| ATOM | 219 | CA | ARG | A | 134 | 57.661 | 9.313 | 123.978 | 1.00 | 20.66 | A | C |
| ATOM | 220 | CB | ARG | A | 134 | 58.084 | 7.961 | 123.411 | 1.00 | 20.98 | A | C |
| ATOM | 221 | CG | ARO | A | 134 | 59.563 | 7.723 | 123.628 | 1.00 | 21.85 | A | C |
| ATOM | 222 | CD | ARO | A | 134 | 60.064 | 6.476 | 122.953 | 1.00 | 25.93 | A | C |
| ATOM | 223 | NE | ARG | A | 134 | 61.506 | 6.346 | 123.137 | 1.00 | 29.18 | A | N |
| ATOM | 224 | CZ | ARG | A | 134 | 62.234 | 5.347 | 122.652 | 1.00 | 32.95 | A | C |
| ATOM | 225 | NH1 | ARG | A | 134 | 61.649 | 4.384 | 121.949 | 1.00 | 31.93 | A | N |
| ATOM | 226 | NH2 | ARG | A | 134 | 63.544 | 5.314 | 122.867 | 1.00 | 31.66 | A | N |
| ATOM | 227 | C | ARG | A | 134 | 56.217 | 9.633 | 123.615 | 1.00 | 20.30 | A | C |
| ATOM | 228 | O | ARG | A | 134 | 55.961 | 10.226 | 122.575 | 1.00 | 19.91 | A | O |
| ATOM | 229 | N | ALA | A | 135 | 55.274 | 9.256 | 124.470 | 1.00 | 19.00 | A | N |
| ATOM | 230 | CA | ALA | A | 135 | 53.871 | 9.552 | 124.201 | 1.00 | 20.03 | A | C |
| ATOM | 231 | CB | ALA | A | 135 | 52.980 | 8.861 | 125.237 | 1.00 | 18.70 | A | C |
| ATOM | 232 | C | ALA | A | 135 | 53.642 | 11.073 | 124.226 | 1.00 | 19.99 | A | C |
| ATOM | 233 | O | ALA | A | 135 | 52.814 | 11.601 | 123.489 | 1.00 | 19.37 | A | O |
| ATOM | 234 | N | VAL | A | 136 | 54.378 | 11.774 | 125.082 | 1.00 | 18.65 | A | N |
| ATOM | 235 | CA | VAL | A | 136 | 54.245 | 13.227 | 125.175 | 1.00 | 18.87 | A | C |
| ATOM | 236 | CB | VAL | A | 136 | 54.950 | 13.777 | 126.459 | 1.00 | 18.18 | A | C |
| ATOM | 237 | CG1 | VAL | A | 136 | 54.999 | 15.309 | 126.443 | 1.00 | 16.50 | A | C |
| ATOM | 238 | CG2 | VAL | A | 136 | 54.204 | 13.303 | 127.688 | 1.00 | 13.02 | A | C |
| ATOM | 239 | C | VAL | A | 136 | 54.838 | 13.900 | 123.930 | 1.00 | 17.85 | A | C |
| ATOM | 240 | O | VAL | A | 136 | 54.277 | 14.870 | 123.400 | 1.00 | 16.07 | A | O |
| ATOM | 241 | N | ILE | A | 137 | 55.969 | 13.374 | 123.469 | 1.00 | 14.93 | A | N |
| ATOM | 242 | CA | ILE | A | 137 | 56.630 | 13.916 | 122.295 | 1.00 | 13.94 | A | C |
| ATOM | 243 | CB | ILE | A | 137 | 58.035 | 13.292 | 122.121 | 1.00 | 13.55 | A | C |
| ATOM | 244 | CG2 | ILE | A | 137 | 58.657 | 13.730 | 120.789 | 1.00 | 10.34 | A | C |
| ATOM | 245 | CG1 | ILE | A | 137 | 58.917 | 13.705 | 123.307 | 1.00 | 11.84 | A | C |
| ATOM | 246 | CD1 | ILE | A | 137 | 60.329 | 13.165 | 123.274 | 1.00 | 8.78 | A | C |
| ATOM | 247 | C | ILE | A | 137 | 55.781 | 13.689 | 121.044 | 1.00 | 13.89 | A | C |
| ATOM | 248 | O | ILE | A | 137 | 55.557 | 14.616 | 120.270 | 1.00 | 13.43 | A | O |
| ATOM | 249 | N | ASP | A | 138 | 55.298 | 12.465 | 120.850 | 1.00 | 14.79 | A | N |
| ATOM | 250 | CA | ASP | A | 138 | 54.469 | 12.168 | 119.685 | 1.00 | 16.05 | A | C |
| ATOM | 251 | CB | ASP | A | 138 | 53.891 | 10.748 | 119.764 | 1.00 | 14.50 | A | C |
| ATOM | 252 | CG | ASP | A | 138 | 54.946 | 9.662 | 119.593 | 1.00 | 16.77 | A | C |
| ATOM | 253 | OD1 | ASP | A | 138 | 56.089 | 9.967 | 119.188 | 1.00 | 18.40 | A | O |
| ATOM | 254 | OD2 | ASP | A | 138 | 54.619 | 8.484 | 119.853 | 1.00 | 20.23 | A | O |
| ATOM | 255 | C | ASP | A | 138 | 53.317 | 13.170 | 119.602 | 1.00 | 16.70 | A | C |
| ATOM | 256 | O | ASP | A | 138 | 53.086 | 13.784 | 118.564 | 1.00 | 19.57 | A | O |
| ATOM | 257 | N | ASP | A | 139 | 52.612 | 13.337 | 120.716 | 1.00 | 16.62 | A | N |
| ATOM | 258 | CA | ASP | A | 139 | 51.460 | 14.232 | 120.801 | 1.00 | 15.09 | A | C |
| ATOM | 259 | CB | ASP | A | 139 | 50.812 | 14.099 | 122.181 | 1.00 | 14.62 | A | C |
| ATOM | 260 | CG | ASP | A | 139 | 49.611 | 15.003 | 122.354 | 1.00 | 16.52 | A | C |
| ATOM | 261 | OD1 | ASP | A | 139 | 48.584 | 14.769 | 121.692 | 1.00 | 19.96 | A | O |
| ATOM | 262 | OD2 | ASP | A | 139 | 49.688 | 15.953 | 123.152 | 1.00 | 12.35 | A | O |
| ATOM | 263 | C | ASP | A | 139 | 51.785 | 15.693 | 120.526 | 1.00 | 15.75 | A | C |
| ATOM | 264 | O | ASP | A | 139 | 51.004 | 16.396 | 119.894 | 1.00 | 17.52 | A | O |
| ATOM | 265 | N | ALA | A | 140 | 52.935 | 16.152 | 121.008 | 1.00 | 16.49 | A | N |
| ATOM | 266 | CA | ALA | A | 140 | 53.356 | 17.536 | 120.805 | 1.00 | 14.14 | A | C |
| ATOM | 267 | CB | ALA | A | 140 | 54.648 | 17.819 | 121.586 | 1.00 | 10.53 | A | C |
| ATOM | 268 | C | ALA | A | 140 | 53.576 | 17.805 | 119.319 | 1.00 | 13.23 | A | C |
| ATOM | 269 | O | ALA | A | 140 | 53.156 | 18.838 | 118.797 | 1.00 | 13.50 | A | O |
| ATOM | 270 | N | PHE | A | 141 | 54.249 | 16.877 | 118.645 | 1.00 | 12.36 | A | N |
| ATOM | 271 | CA | PHE | A | 141 | 54.501 | 17.017 | 117.216 | 1.00 | 15.20 | A | C |
| ATOM | 272 | CB | PHE | A | 141 | 55.431 | 15.901 | 116.722 | 1.00 | 13.79 | A | C |
| ATOM | 273 | CG | PHE | A | 141 | 56.867 | 16.073 | 117.146 | 1.00 | 11.40 | A | C |
| ATOM | 274 | CD1 | PHE | A | 141 | 57.408 | 17.340 | 117.318 | 1.00 | 12.07 | A | C |
| ATOM | 275 | CD2 | PHE | A | 141 | 57.685 | 14.973 | 117.341 | 1.00 | 11.50 | A | C |
| ATOM | 276 | GE1 | PHE | A | 141 | 58.752 | 17.507 | 117.678 | 1.00 | 15.23 | A | C |
| ATOM | 277 | CE2 | PHB | A | 141 | 59.032 | 15.133 | 117.701 | 1.00 | 10.84 | A | C |
| ATOM | 278 | CZ | PHB | A | 141 | 59.564 | 16.396 | 117.868 | 1.00 | 9.69 | A | C |
| ATOM | 279 | C | PHB | A | 141 | 53.183 | 16.988 | 116.433 | 1.00 | 15.88 | A | C |
| ATOM | 280 | O | PHE | A | 141 | 52.980 | 17.783 | 115.516 | 1.00 | 18.45 | A | O |
| ATOM | 281 | N | ALA | A | 142 | 52.289 | 16.080 | 116.815 | 1.00 | 15.46 | A | N |
| ATOM | 282 | CA | ALA | A | 142 | 50.997 | 15.940 | 116.156 | 1.00 | 16.51 | A | C |
| ATOM | 283 | CB | ALA | A | 142 | 50.235 | 14.769 | 116.751 | 1.00 | 16.42 | A | C |
| ATOM | 284 | C | ALA | A | 142 | 50.175 | 17.217 | 116.281 | 1.00 | 17.88 | A | C |
| ATOM | 285 | O | ALA | A | 142 | 49.519 | 17.642 | 115.327 | 1.00 | 14.95 | A | O |
| ATOM | 286 | N | ARG | A | 143 | 50.207 | 17.826 | 117.464 | 1.00 | 17.58 | A | N |
| ATOM | 287 | CA | ARG | A | 143 | 49.465 | 19.055 | 117.687 | 1.00 | 16.25 | A | C |
| ATOM | 288 | CB | ARG | A | 143 | 49.349 | 19.353 | 119.191 | 1.00 | 13.94 | A | C |
| ATOM | 289 | CG | ARG | A | 143 | 48.421 | 18.387 | 119.930 | 1.00 | 13.87 | A | C |
| ATOM | 290 | CD | ARG | A | 143 | 48.231 | 18.747 | 121.404 | 1.00 | 13.42 | A | C |
| ATOM | 291 | NE | ARG | A | 143 | 49.470 | 18.634 | 122.168 | 1.00 | 12.17 | A | N |
| ATOM | 292 | CZ | ARG | A | 143 | 50.164 | 19.671 | 122.631 | 1.00 | 14.73 | A | C |
| ATOM | 293 | NH1 | ARG | A | 143 | 49.732 | 20.910 | 122.416 | 1.00 | 12.72 | A | N |
| ATOM | 294 | NH2 | ARG | A | 143 | 51.305 | 19.471 | 123.287 | 1.00 | 14.62 | A | N |
| ATOM | 295 | C | ARG | A | 143 | 50.139 | 20.214 | 116.952 | 1.00 | 16.25 | A | C |
| ATOM | 296 | O | ARG | A | 143 | 49.478 | 21.162 | 116.535 | 1.00 | 18.41 | A | O |
| ATOM | 297 | N | ALA | A | 144 | 51.453 | 20.140 | 116.787 | 1.00 | 14.76 | A | N |
| ATOM | 298 | CA | ALA | A | 144 | 52.165 | 21.199 | 116.079 | 1.00 | 14.81 | A | C |
| ATOM | 299 | CB | ALA | A | 144 | 53.687 | 21.021 | 116.247 | 1.00 | 9.68 | A | C |
| ATOM | 300 | C | ALA | A | 144 | 51.763 | 21.169 | 114.592 | 1.00 | 15.68 | A | C |
| ATOM | 301 | O | ALA | A | 144 | 51.588 | 22.214 | 113.970 | 1.00 | 14.69 | A | O |
| ATOM | 302 | N | PHE | A | 145 | 51.605 | 19.968 | 114.033 | 1.00 | 16.80 | A | N |
| ATOM | 303 | CA | PHE | A | 145 | 51.193 | 19.815 | 112.632 | 1.00 | 17.22 | A | C |
| ATOM | 304 | CB | PHE | A | 145 | 51.386 | 18.367 | 112.154 | 1.00 | 14.07 | A | C |
| ATOM | 305 | CG | PHE | A | 145 | 52.791 | 18.045 | 111.744 | 1.00 | 9.59 | A | C |
| ATOM | 306 | CD1 | PHE | A | 145 | 53.433 | 18.797 | 110.767 | 1.00 | 10.98 | A | C |
| ATOM | 307 | CD2 | PHE | A | 145 | 53.472 | 16.990 | 112.326 | 1.00 | 9.63 | A | C |
| ATOM | 308 | CE1 | PEE | A | 145 | 54.739 | 18.503 | 110.373 | 1.00 | 10.38 | A | C |
| ATOM | 309 | CE2 | PHE | A | 145 | 54.781 | 16.687 | 111.939 | 1.00 | 11.84 | A | C |
| ATOM | 310 | CZ | PEE | A | 145 | 55.414 | 17.448 | 110.958 | 1.00 | 9.71 | A | C |
| ATOM | 311 | C | PEB | A | 145 | 49.729 | 20.209 | 112.438 | 1.00 | 19.32 | A | C |
| ATOM | 312 | O | PHE | A | 145 | 49.366 | 20.790 | 111.414 | 1.00 | 20.25 | A | O |
| ATOM | 313 | N | ALA | A | 146 | 48.889 | 19.891 | 113.420 | 1.00 | 18.99 | A | N |
| ATOM | 314 | CA | ALA | A | 146 | 47.471 | 20.224 | 113.330 | 1.00 | 20.15 | A | C |
| ATOM | 315 | CB | ALA | A | 146 | 46.730 | 19.695 | 114.542 | 1.00 | 16.75 | A | C |
| ATOM | 316 | C | ALA | A | 146 | 47.246 | 21.727 | 113.195 | 1.00 | 21.23 | A | C |
| ATOM | 317 | O | ALA | A | 146 | 46.272 | 22.153 | 112.592 | 1.00 | 24.43 | A | O |
| ATOM | 318 | N | LEU | A | 147 | 48.137 | 22.533 | 113.764 | 1.00 | 23.09 | A | N |
| ATOM | 319 | CA | LEU | A | 147 | 47.996 | 23.983 | 113.668 | 1.00 | 21.92 | A | C |
| ATOM | 320 | CB | LEU | A | 147 | 49.155 | 24.698 | 114.367 | 1.00 | 20.86 | A | C |
| ATOM | 321 | CG | LEU | A | 147 | 49.260 | 24.748 | 115.894 | 1.00 | 24.92 | A | C |
| ATOM | 322 | CD1 | LEU | A | 147 | 50.469 | 25.598 | 116.255 | 1.00 | 22.84 | A | C |
| ATOM | 323 | CD2 | LEU | A | 147 | 48.001 | 25.342 | 116.513 | 1.00 | 22.59 | A | C |
| ATOM | 324 | C | LEU | A | 147 | 47.984 | 24.422 | 112.206 | 1.00 | 22.21 | A | C |
| ATOM | 325 | O | LEU | A | 147 | 47.188 | 25.265 | 111.797 | 1.00 | 22.01 | A | O |
| ATOM | 326 | N | TRP | A | 148 | 48.884 | 23.842 | 111.425 | 1.00 | 21.86 | A | N |
| ATOM | 327 | CA | TRP | A | 148 | 49.013 | 24.196 | 110.026 | 1.00 | 23.11 | A | C |
| ATOM | 328 | CB | TRP | A | 148 | 50.413 | 23.831 | 109.534 | 1.00 | 19.89 | A | C |
| ATOM | 329 | CG | TRP | A | 148 | 51.513 | 24.569 | 110.249 | 1.00 | 21.33 | A | C |
| ATOM | 330 | CD2 | TRP | A | 148 | 51.827 | 25.964 | 110.138 | 1.00 | 21.93 | A | C |
| ATOM | 331 | CE2 | TRP | A | 148 | 52.965 | 26.203 | 110.942 | 1.00 | 20.27 | A | C |
| ATOM | 332 | CE3 | TRP | A | 148 | 51.259 | 27.036 | 109.435 | 1.00 | 18.40 | A | C |
| ATOM | 333 | CD1 | TRP | A | 148 | 52.445 | 24.037 | 111.100 | 1.00 | 20.02 | A | C |
| ATOM | 334 | NE1 | TRP | A | 148 | 53.320 | 25.012 | 111.517 | 1.00 | 17.63 | A | N |
| ATOM | 335 | CZ2 | TRP | A | 148 | 53.548 | 27.472 | 111.059 | 1.00 | 19.95 | A | C |
| ATOM | 336 | CZ3 | TRP | A | 148 | 51.838 | 28.297 | 109.553 | 1.00 | 19.74 | A | C |
| ATOM | 337 | CH2 | TRP | A | 148 | 52.972 | 28.503 | 110.359 | 1.00 | 18.39 | A | C |
| ATOM | 338 | C | TRP | A | 148 | 47.955 | 23.571 | 109.126 | 1.00 | 23.44 | A | C |
| ATOM | 339 | O | TRP | A | 148 | 47.419 | 24.237 | 108.244 | 1.00 | 21.73 | A | O |
| ATOM | 340 | N | SER | A | 149 | 47.645 | 22.300 | 109.351 | 1.00 | 25.55 | A | N |
| ATOM | 341 | CA | SER | A | 149 | 46.648 | 21.630 | 108.531 | 1.00 | 27.95 | A | C |
| ATOM | 342 | CB | SER | A | 149 | 46.618 | 20.127 | 108.823 | 1.00 | 28.12 | A | C |
| ATOM | 343 | OG | SER | A | 149 | 46.132 | 19.877 | 110.124 | 1.00 | 27.95 | A | O |
| ATOM | 344 | C | SER | A | 149 | 45.274 | 22.220 | 108.782 | 1.00 | 28.65 | A | C |
| ATOM | 345 | O | SER | A | 149 | 44.340 | 21.959 | 108.031 | 1.00 | 30.51 | A | O |
| ATOM | 346 | N | ALA | A | 150 | 45.148 | 23.022 | 109.833 | 1.00 | 27.46 | A | N |
| ATOM | 347 | CA | ALA | A | 150 | 43.864 | 23.627 | 110.151 | 1.00 | 27.51 | A | C |
| ATOM | 348 | CB | ALA | A | 150 | 43.817 | 24.013 | 111.620 | 1.00 | 24.47 | A | C |
| ATOM | 349 | C | ALA | A | 150 | 43.552 | 24.843 | 109.280 | 1.00 | 28.60 | A | C |
| ATOM | 350 | O | ALA | A | 150 | 42.393 | 25.187 | 109.091 | 1.00 | 28.82 | A | O |
| ATOM | 351 | N | VAL | A | 151 | 44.579 | 25.492 | 108.745 | 1.00 | 30.95 | A | N |
| ATOM | 352 | CA | VAL | A | 151 | 44.366 | 26.670 | 107.909 | 1.00 | 30.77 | A | C |
| ATOM | 353 | CB | VAL | A | 151 | 45.144 | 27.891 | 108.456 | 1.00 | 30.72 | A | C |
| ATOM | 354 | CO1 | VAL | A | 151 | 44.557 | 28.320 | 109.784 | 1.00 | 32.55 | A | C |
| ATOM | 355 | CG2 | VAL | A | 151 | 46.617 | 27.546 | 108.623 | 1.00 | 27.92 | A | C |
| ATOM | 356 | C | VAL | A | 151 | 44.765 | 26.451 | 106.455 | 1.00 | 30.85 | A | C |
| ATOM | 357 | O | VAL | A | 151 | 44.898 | 27.405 | 105.691 | 1.00 | 31.86 | A | O |
| ATOM | 358 | N | THR | A | 152 | 44.958 | 25.195 | 106.072 | 1.00 | 30.06 | A | N |
| ATOM | 359 | CA | THR | A | 152 | 45.339 | 24.879 | 104.703 | 1.00 | 30.51 | A | C |
| ATOM | 360 | CB | THR | A | 152 | 46.879 | 24.805 | 104.536 | 1.00 | 29.92 | A | C |
| ATOM | 361 | OG1 | THR | A | 152 | 47.390 | 23.686 | 105.273 | 1.00 | 28.72 | A | O |
| ATOM | 362 | CG2 | THR | A | 152 | 47.538 | 26.087 | 105.021 | 1.00 | 30.53 | A | C |
| ATOM | 363 | C | THR | A | 152 | 44.760 | 23.530 | 104.301 | 1.00 | 30.44 | A | C |
| ATOM | 364 | O | THR | A | 152 | 44.171 | 22.831 | 105.125 | 1.00 | 29.70 | A | O |
| ATOM | 365 | N | PRO | A | 153 | 44.904 | 23.157 | 103.017 | 1.00 | 30.47 | A | N |
| ATOM | 366 | CD | PRO | A | 153 | 45.298 | 24.023 | 101.887 | 1.00 | 30.45 | A | C |
| ATOM | 367 | CA | PRO | A | 153 | 44.392 | 21.875 | 102.527 | 1.00 | 30.14 | A | C |
| ATOM | 368 | CB | PRO | A | 153 | 44.174 | 22.145 | 101.042 | 1.00 | 30.77 | A | C |
| ATOM | 369 | CG | PRO | A | 153 | 45.304 | 23.059 | 100.717 | 1.00 | 32.01 | A | C |
| ATOM | 370 | C | PRO | A | 153 | 45.400 | 20.750 | 102.768 | 1.00 | 29.51 | A | C |
| ATOM | 371 | O | PRO | A | 153 | 45.190 | 19.619 | 102.336 | 1.00 | 30.25 | A | O |
| ATOM | 372 | N | LEU | A | 154 | 46.496 | 21.075 | 103.451 | 1.00 | 27.86 | A | N |
| ATOM | 373 | CA | LEU | A | 154 | 47.539 | 20.100 | 103.753 | 1.00 | 26.19 | A | C |
| ATOM | 374 | CB | LEU | A | 154 | 48.808 | 20.805 | 104.233 | 1.00 | 26.31 | A | C |
| ATOM | 375 | CO | LEU | A | 154 | 49.491 | 21.847 | 103.342 | 1.00 | 25.08 | A | C |
| ATOM | 376 | CD1 | LEU | A | 154 | 50.606 | 22.524 | 104.124 | 1.00 | 23.11 | A | C |
| ATOM | 377 | CD2 | LEU | A | 154 | 50.043 | 21.196 | 102.097 | 1.00 | 26.17 | A | C |
| ATOM | 378 | C | LEU | A | 154 | 47.104 | 19.116 | 104.832 | 1.00 | 25.56 | A | C |
| ATOM | 379 | O | LEU | A | 154 | 46.290 | 19.440 | 105.693 | 1.00 | 26.20 | A | O |
| ATOM | 380 | N | THR | A | 155 | 47.646 | 17.908 | 104.764 | 1.00 | 24.90 | A | N |
| ATOM | 381 | CA | THR | A | 155 | 47.363 | 16.875 | 105.752 | 1.00 | 26.31 | A | C |
| ATOM | 382 | CB | THR | A | 155 | 46.481 | 15.746 | 105.190 | 1.00 | 27.72 | A | C |
| ATOM | 383 | OG1 | THR | A | 155 | 47.094 | 15.203 | 104.014 | 1.00 | 32.34 | A | O |
| ATOM | 384 | CG2 | THR | A | 155 | 45.088 | 16.267 | 104.862 | 1.00 | 27.86 | A | C |
| ATOM | 385 | C | THR | A | 155 | 48.711 | 16.294 | 106.134 | 1.00 | 25.34 | A | C |
| ATOM | 386 | O | THR | A | 155 | 49.670 | 16.379 | 105.360 | 1.00 | 23.30 | A | O |
| ATOM | 387 | N | PHE | A | 156 | 48.790 | 15.707 | 107.323 | 1.00 | 23.87 | A | N |
| ATOM | 388 | CA | PHE | A | 156 | 50.044 | 15.139 | 107.787 | 1.00 | 22.82 | A | C |
| ATOM | 389 | CB | PHE | A | 156 | 50.630 | 16.046 | 108.870 | 1.00 | 19.73 | A | C |
| ATOM | 390 | CG | PHE | A | 156 | 50.935 | 17.434 | 108.375 | 1.00 | 15.72 | A | C |
| ATOM | 391 | CD1 | PHE | A | 156 | 52.108 | 17.693 | 107.676 | 1.00 | 15.50 | A | C |
| ATOM | 392 | CD2 | PHE | A | 156 | 50.011 | 18.461 | 108.536 | 1.00 | 16.19 | A | C |
| ATOM | 393 | CE1 | PHE | A | 156 | 52.357 | 18.952 | 107.139 | 1.00 | 14.40 | A | C |
| ATOM | 394 | CE2 | PHE | A | 156 | 50.249 | 19.725 | 108.004 | 1.00 | 15.79 | A | C |
| ATOM | 395 | CZ | PHE | A | 156 | 51.423 | 19.972 | 107.304 | 1.00 | 15.01 | A | C |
| ATOM | 396 | C | PHE | A | 156 | 49.864 | 13.718 | 108.286 | 1.00 | 24.14 | A | C |
| ATOM | 397 | O | PHE | A | 156 | 49.026 | 13.441 | 109.145 | 1.00 | 26.34 | A | O |
| ATOM | 398 | N | THR | A | 157 | 50.660 | 12.819 | 107.726 | 1.00 | 24.42 | A | N |
| ATOM | 399 | CA | THR | A | 157 | 50.595 | 11.410 | 108.070 | 1.00 | 23.69 | A | C |
| ATOM | 400 | CB | THR | A | 157 | 50.245 | 10.569 | 106.822 | 1.00 | 23.41 | A | C |
| ATOM | 401 | OG1 | THR | A | 157 | 48.935 | 10.931 | 106.373 | 1.00 | 25.91 | A | O |
| ATOM | 402 | CG2 | THR | A | 157 | 50.277 | 9.081 | 107.135 | 1.00 | 21.00 | A | C |
| ATOM | 403 | C | THR | A | 157 | 51.915 | 10.939 | 108.638 | 1.00 | 22.11 | A | C |
| ATOM | 404 | O | THR | A | 157 | 52.984 | 11.228 | 108.090 | 1.00 | 21.92 | A | O |
| ATOM | 405 | N | ARG | A | 158 | 51.830 | 10.214 | 109.745 | 1.00 | 22.59 | A | N |
| ATOM | 406 | CA | ARG | A | 158 | 53.015 | 9.679 | 110.394 | 1.00 | 22.35 | A | C |
| ATOM | 407 | CB | ARG | A | 158 | 52.780 | 9.534 | 111.899 | 1.00 | 21.18 | A | C |
| ATOM | 408 | CG | ARG | A | 158 | 53.998 | 9.010 | 112.630 | 1.00 | 21.22 | A | C |
| ATOM | 409 | CD | ARG | A | 158 | 53.758 | 8.843 | 114.113 | 1.00 | 20.30 | A | C |
| ATOM | 410 | NE | ARG | A | 158 | 54.959 | 8.340 | 114.776 | 1.00 | 20.99 | A | N |
| ATOM | 411 | CZ | ARG | A | 158 | 55.013 | 7.974 | 116.051 | 1.00 | 20.64 | A | C |
| ATOM | 412 | NH1 | ARG | A | 158 | 53.931 | 8.058 | 116.811 | 1.00 | 19.84 | A | N |
| ATOM | 413 | NH2 | ARG | A | 158 | 56.147 | 7.506 | 116.560 | 1.00 | 20.74 | A | N |
| ATOM | 414 | C | ARG | A | 158 | 53.362 | 8.320 | 109.802 | 1.00 | 21.92 | A | C |
| ATOM | 415 | O | ARG | A | 158 | 52.508 | 7.442 | 109.710 | 1.00 | 21.61 | A | O |
| ATOM | 416 | N | VAL | A | 159 | 54.618 | 8.156 | 109.399 | 1.00 | 24.22 | A | N |
| ATOM | 417 | CA | VAL | A | 159 | 55.092 | 6.901 | 108.823 | 1.00 | 25.52 | A | C |
| ATOM | 418 | CB | VAL | A | 159 | 55.403 | 7.059 | 107.311 | 1.00 | 23.23 | A | C |
| ATOM | 419 | CO1 | VAL | A | 159 | 54.147 | 7.523 | 106.573 | 1.00 | 19.77 | A | C |
| ATOM | 420 | CG2 | VAL | A | 159 | 56.539 | 8.045 | 107.109 | 1.00 | 21.91 | A | C |
| ATOM | 421 | C | VAL | A | 159 | 56.344 | 6.469 | 109.579 | 1.00 | 28.57 | A | C |
| ATOM | 422 | O | VAL | A | 159 | 56.842 | 7.212 | 110.424 | 1.00 | 29.72 | A | O |
| ATOM | 423 | N | TYR | A | 160 | 56.859 | 5.278 | 109.282 | 1.00 | 31.79 | A | N |
| ATOM | 424 | CA | TYR | A | 160 | 58.033 | 4.791 | 109.995 | 1.00 | 33.61 | A | C |
| ATOM | 425 | CB | TYR | A | 160 | 57.634 | 3.596 | 110.863 | 1.00 | 29.27 | A | C |
| ATOM | 426 | CO | TYR | A | 160 | 56.570 | 3.952 | 111.873 | 1.00 | 28.93 | A | C |
| ATOM | 427 | CD1 | TYR | A | 160 | 55.230 | 4.056 | 111.498 | 1.00 | 28.23 | A | C |
| ATOM | 428 | CE2 | TYR | A | 160 | 54.256 | 4.465 | 112.410 | 1.00 | 29.32 | A | C |
| ATOM | 429 | CD2 | TYR | A | 160 | 56.912 | 4.261 | 113.191 | 1.00 | 28.64 | A | C |
| ATOM | 430 | CE2 | TYR | A | 160 | 55.949 | 4.670 | 114.108 | 1.00 | 29.60 | A | C |
| ATOM | 431 | CZ | TYR | A | 160 | 54.626 | 4.772 | 113.712 | 1.00 | 30.04 | A | C |
| ATOM | 432 | OH | TYR | A | 160 | 53.680 | 5.193 | 114.616 | 1.00 | 32.56 | A | O |
| ATOM | 433 | C | TYR | A | 160 | 59.270 | 4.449 | 109.170 | 1.00 | 36.31 | A | C |
| ATOM | 434 | O | TYR | A | 160 | 60.097 | 3.640 | 109.591 | 1.00 | 39.18 | A | O |
| ATOM | 435 | N | SER | A | 161 | 59.414 | 5.075 | 108.010 | 1.00 | 37.86 | A | N |
| ATOM | 436 | CA | SER | A | 161 | 60.574 | 4.816 | 107.174 | 1.00 | 39.80 | A | C |
| ATOM | 437 | CB | SER | A | 161 | 60.166 | 4.018 | 105.933 | 1.00 | 38.63 | A | C |
| ATOM | 438 | OG | SER | A | 161 | 59.238 | 4.737 | 105.143 | 1.00 | 39.02 | A | O |
| ATOM | 439 | C | SER | A | 161 | 61.231 | 6.128 | 106.766 | 1.00 | 41.58 | A | C |
| ATOM | 440 | O | SER | A | 161 | 60.664 | 7.200 | 106.972 | 1.00 | 41.79 | A | O |
| ATOM | 441 | N | ARG | A | 162 | 62.426 | 6.033 | 106.187 | 1.00 | 44.11 | A | N |
| ATOM | 442 | CA | ARG | A | 162 | 63.188 | 7.198 | 105.739 | 1.00 | 46.57 | A | C |
| ATOM | 443 | CB | ARG | A | 162 | 64.613 | 6.790 | 105.350 | 1.00 | 48.67 | A | C |
| ATOM | 444 | CG | ARG | A | 162 | 65.408 | 6.045 | 106.418 | 1.00 | 53.36 | A | C |
| ATOM | 445 | CD | ARO | A | 162 | 64.757 | 4.721 | 106.797 | 1.00 | 57.23 | A | C |
| ATOM | 446 | NE | ARG | A | 162 | 65.661 | 3.839 | 107.528 | 1.00 | 60.31 | A | N |
| ATOM | 447 | CZ | ARG | A | 162 | 65.274 | 2.728 | 108.145 | 1.00 | 62.63 | A | C |
| ATOM | 448 | NH1 | ARG | A | 162 | 63.997 | 2.372 | 108.120 | 1.00 | 63.79 | A | N |
| ATOM | 449 | NH2 | ARG | A | 162 | 66.161 | 1.965 | 108.777 | 1.00 | 62.58 | A | N |
| ATOM | 450 | C | ARG | A | 162 | 62.537 | 7.862 | 104.529 | 1.00 | 47.42 | A | C |
| ATOM | 451 | O | ARG | A | 162 | 63.027 | 8.879 | 104.040 | 1.00 | 48.56 | A | O |
| ATOM | 452 | N | ASP | A | 163 | 61.441 | 7.282 | 104.048 | 1.00 | 48.01 | A | N |
| ATOM | 453 | CA | ASP | A | 163 | 60.739 | 7.804 | 102.876 | 1.00 | 46.94 | A | C |
| ATOM | 454 | CB | ASP | A | 163 | 59.909 | 6.695 | 102.227 | 1.00 | 52.93 | A | C |
| ATOM | 455 | CG | ASP | A | 163 | 60.766 | 5.584 | 101.665 | 1.00 | 56.71 | A | C |
| ATOM | 456 | OD1 | ASP | A | 163 | 61.681 | 5.891 | 100.871 | 1.00 | 59.29 | A | O |
| ATOM | 457 | OD2 | ASP | A | 163 | 60.525 | 4.409 | 102.013 | 1.00 | 61.03 | A | O |
| ATOM | 458 | C | ASP | A | 163 | 59.838 | 9.008 | 103.127 | 1.00 | 42.48 | A | C |
| ATOM | 459 | O | ASP | A | 163 | 59.350 | 9.627 | 102.181 | 1.00 | 43.42 | A | O |
| ATOM | 460 | N | ALA | A | 164 | 59.612 | 9.337 | 104.392 | 1.00 | 35.33 | A | N |
| ATOM | 461 | CA | ALA | A | 164 | 58.768 | 10.476 | 104.738 | 1.00 | 28.12 | A | C |
| ATOM | 462 | CB | ALA | A | 164 | 58.656 | 10.591 | 106.254 | 1.00 | 28.52 | A | C |
| ATOM | 463 | C | ALA | A | 164 | 59.332 | 11.776 | 104.159 | 1.00 | 24.32 | A | C |
| ATOM | 464 | O | ALA | A | 164 | 60.528 | 11.868 | 103.859 | 1.00 | 18.94 | A | O |
| ATOM | 465 | N | ASP | A | 165 | 58.468 | 12.776 | 103.996 | 1.00 | 20.55 | A | N |
| ATOM | 466 | CA | ASP | A | 165 | 58.910 | 14.070 | 103.491 | 1.00 | 19.96 | A | C |
| ATOM | 467 | CB | ASP | A | 165 | 57.732 | 14.931 | 103.033 | 1.00 | 20.58 | A | C |
| ATOM | 468 | CG | ASP | A | 165 | 56.953 | 14.302 | 101.918 | 1.00 | 19.34 | A | C |
| ATOM | 469 | OD1 | ASP | A | 165 | 57.577 | 13.931 | 100.907 | 1.00 | 22.92 | A | O |
| ATOM | 470 | OD2 | ASP | A | 165 | 55.720 | 14.185 | 102.053 | 1.00 | 20.26 | A | O |
| ATOM | 471 | C | ASP | A | 165 | 59.619 | 14.808 | 104.609 | 1.00 | 19.50 | A | C |
| ATOM | 472 | O | ASP | A | 165 | 60.669 | 15.387 | 104.403 | 1.00 | 20.89 | A | O |
| ATOM | 473 | N | ILE | A | 166 | 59.031 | 14.798 | 105.798 | 1.00 | 18.88 | A | N |
| ATOM | 474 | CA | ILE | A | 166 | 59.638 | 15.496 | 106.923 | 1.00 | 18.31 | A | C |
| ATOM | 475 | CB | ILE | A | 166 | 58.612 | 16.435 | 107.598 | 1.00 | 16.76 | A | C |
| ATOM | 476 | CG2 | ILE | A | 166 | 59.241 | 17.114 | 108.817 | 1.00 | 15.02 | A | C |
| ATOM | 477 | CG1 | ILE | A | 166 | 58.129 | 17.477 | 106.581 | 1.00 | 16.47 | A | C |
| ATOM | 478 | CD1 | ILE | A | 166 | 57.004 | 18.367 | 107.073 | 1.00 | 12.28 | A | C |
| ATOM | 479 | C | ILE | A | 166 | 60.198 | 14.523 | 107.950 | 1.00 | 18.31 | A | C |
| ATOM | 480 | O | ILE | A | 166 | 59.448 | 13.831 | 108.635 | 1.00 | 19.42 | A | O |
| ATOM | 481 | N | VAL | A | 167 | 61.522 | 14.453 | 108.032 | 1.00 | 16.42 | A | N |
| ATOM | 482 | CA | VAL | A | 167 | 62.172 | 13.577 | 108.992 | 1.00 | 14.89 | A | C |
| ATOM | 483 | GB | VAL | A | 167 | 63.410 | 12.891 | 108.391 | 1.00 | 16.42 | A | C |
| ATOM | 484 | CO1 | VAL | A | 167 | 64.133 | 12.076 | 109.464 | 1.00 | 14.30 | A | C |
| ATOM | 485 | CG2 | VAL | A | 167 | 62.984 | 11.982 | 107.243 | 1.00 | 18.08 | A | C |
| ATOM | 486 | C | VAL | A | 167 | 62.581 | 14.415 | 110.200 | 1.00 | 17.08 | A | C |
| ATOM | 487 | O | VAL | A | 167 | 63.232 | 15.456 | 110.064 | 1.00 | 13.46 | A | O |
| ATOM | 488 | N | ILE | A | 168 | 62.183 | 13.943 | 111.378 | 1.00 | 15.49 | A | N |
| ATOM | 489 | CA | ILE | A | 168 | 62.445 | 14.624 | 112.633 | 1.00 | 16.01 | A | C |
| ATOM | 490 | CB | ILE | A | 168 | 61.145 | 14.735 | 113.448 | 1.00 | 13.64 | A | C |
| ATOM | 491 | CG2 | ILE | A | 168 | 61.406 | 15.473 | 114.755 | 1.00 | 14.98 | A | C |
| ATOM | 492 | CG1 | ILE | A | 168 | 60.077 | 15.427 | 112.603 | 1.00 | 12.69 | A | C |
| ATOM | 493 | CD1 | ILE | A | 168 | 58.685 | 15.344 | 113.170 | 1.00 | 10.54 | A | C |
| ATOM | 494 | C | ILE | A | 168 | 63.482 | 13.906 | 113.486 | 1.00 | 16.29 | A | C |
| ATOM | 495 | O | ILE | A | 168 | 63.442 | 12.693 | 113.648 | 1.00 | 18.46 | A | O |
| ATOM | 496 | N | GLN | A | 169 | 64.414 | 14.656 | 114.045 | 1.00 | 19.10 | A | N |
| ATOM | 497 | CA | GLN | A | 169 | 65.407 | 14.028 | 114.890 | 1.00 | 20.39 | A | C |
| ATOM | 498 | CB | GLN | A | 169 | 66.536 | 13.443 | 114.043 | 1.00 | 23.58 | A | C |
| ATOM | 499 | CG | GLN | A | 169 | 67.428 | 14.481 | 113.409 | 1.00 | 28.43 | A | C |
| ATOM | 500 | CD | GLN | A | 169 | 68.723 | 13.890 | 112.901 | 1.00 | 32.40 | A | C |
| ATOM | 501 | OE1 | GLN | A | 169 | 69.551 | 13.411 | 113.678 | 1.00 | 36.26 | A | O |
| ATOM | 502 | NE2 | GLN | A | 169 | 68.908 | 13.917 | 111.591 | 1.00 | 33.78 | A | N |
| ATOM | 503 | C | GLN | A | 169 | 65.998 | 14.991 | 115.905 | 1.00 | 19.32 | A | C |
| ATOM | 504 | O | GLN | A | 169 | 66.009 | 16.206 | 115.694 | 1.00 | 17.87 | A | O |
| ATOM | 505 | N | PHE | A | 170 | 66.482 | 14.423 | 117.005 | 1.00 | 15.27 | A | N |
| ATOM | 506 | CA | PHE | A | 170 | 67.124 | 15.180 | 118.062 | 1.00 | 16.05 | A | C |
| ATOM | 507 | CB | PHE | A | 170 | 66.613 | 14.740 | 119.444 | 1.00 | 13.11 | A | C |
| ATOM | 508 | CG | PHE | A | 170 | 65.164 | 15.056 | 119.690 | 1.00 | 11.58 | A | C |
| ATOM | 509 | CD1 | PHE | A | 170 | 64.157 | 14.272 | 119.131 | 1.00 | 9.60 | A | C |
| ATOM | 510 | CD2 | PHE | A | 170 | 64.807 | 16.148 | 120.475 | 1.00 | 11.94 | A | C |
| ATOM | 511 | CE1 | PHE | A | 170 | 62.817 | 14.572 | 119.352 | 1.00 | 10.10 | A | C |
| ATOM | 512 | CE2 | PHE | A | 170 | 63.466 | 16.458 | 120.701 | 1.00 | 11.51 | A | C |
| ATOM | 513 | CZ | PHE | A | 170 | 62.471 | 15.671 | 120.141 | 1.00 | 11.15 | A | C |
| ATOM | 514 | C | PHE | A | 170 | 68.611 | 14.873 | 117.953 | 1.00 | 17.13 | A | C |
| ATOM | 515 | O | PHE | A | 170 | 68.998 | 13.728 | 117.747 | 1.00 | 18.15 | A | O |
| ATOM | 516 | N | GLY | A | 171 | 69.450 | 15.889 | 118.082 | 1.00 | 17.88 | A | N |
| ATOM | 517 | CA | GLY | A | 271 | 70.879 | 15.655 | 118.006 | 1.00 | 19.53 | A | C |
| ATOM | 518 | C | GLY | A | 171 | 71.642 | 16.630 | 118.878 | 1.00 | 21.64 | A | C |
| ATOM | 519 | O | GLY | A | 171 | 71.083 | 17.631 | 119.324 | 1.00 | 22.76 | A | O |
| ATOM | 520 | N | VAL | A | 172 | 72.912 | 16.332 | 119.138 | 1.00 | 23.09 | A | N |
| ATOM | 521 | CA | VAL | A | 172 | 73.756 | 17.205 | 119.950 | 1.00 | 25.50 | A | C |
| ATOM | 522 | CB | VAL | A | 172 | 74.109 | 16.560 | 121.326 | 1.00 | 26.03 | A | C |
| ATOM | 523 | CG1 | VAL | A | 172 | 72.844 | 16.252 | 122.083 | 1.00 | 26.98 | A | C |
| ATOM | 524 | CG2 | VAL | A | 172 | 74.918 | 15.292 | 121.134 | 1.00 | 25.99 | A | C |
| ATOM | 525 | C | VAL | A | 172 | 75.038 | 17.489 | 119.174 | 1.00 | 26.07 | A | C |
| ATOM | 526 | O | VAL | A | 172 | 75.653 | 16.574 | 118.615 | 1.00 | 24.44 | A | O |
| ATOM | 527 | N | ALA | A | 173 | 75.427 | 18.760 | 119.139 | 1.00 | 27.68 | A | N |
| ATOM | 528 | CA | ALA | A | 173 | 76.618 | 19.184 | 118.411 | 1.00 | 28.80 | A | C |
| ATOM | 529 | CB | ALA | A | 173 | 77.868 | 18.664 | 119.111 | 1.00 | 29.58 | A | C |
| ATOM | 530 | C | ALA | A | 173 | 76.548 | 18.659 | 116.974 | 1.00 | 29.57 | A | C |
| ATOM | 531 | O | ALA | A | 173 | 75.538 | 18.843 | 116.289 | 1.00 | 27.64 | A | U |
| ATOM | 532 | N | GLU | A | 174 | 77.619 | 18.006 | 116.530 | 1.00 | 29.27 | A | N |
| ATOM | 533 | CA | GLU | A | 174 | 77.699 | 17.437 | 115.189 | 1.00 | 32.29 | A | C |
| ATOM | 534 | CB | GLU | A | 174 | 79.078 | 16.815 | 114.972 | 1.00 | 41.88 | A | C |
| ATOM | 535 | CG | GLU | A | 174 | 80.209 | 17.511 | 115.722 | 1.00 | 51.63 | A | C |
| ATOM | 536 | CD | GLU | A | 174 | 80.512 | 18.892 | 115.178 | 1.00 | 57.77 | A | C |
| ATOM | 537 | OE1 | GLU | A | 174 | 81.379 | 19.584 | 115.757 | 1.00 | 58.88 | A | O |
| ATOM | 538 | OE2 | GLU | A | 174 | 79.888 | 19.284 | 114.167 | 1.00 | 62.24 | A | C |
| ATOM | 539 | C | GLU | A | 174 | 76.657 | 16.340 | 115.076 | 1.00 | 30.20 | A | C |
| ATOM | 540 | O | GLU | A | 174 | 76.668 | 15.409 | 115.874 | 1.00 | 31.68 | A | O |
| ATOM | 541 | N | HIS | A | 175 | 75.768 | 16.418 | 114.092 | 1.00 | 27.49 | A | N |
| ATOM | 542 | CA | HIS | A | 175 | 74.748 | 15.385 | 113.979 | 1.00 | 27.72 | A | C |
| ATOM | 543 | CB | HIS | A | 175 | 73.473 | 15.827 | 114.688 | 1.00 | 24.97 | A | C |
| ATOM | 544 | CG | HIS | A | 175 | 72.928 | 17.118 | 114.175 | 1.00 | 20.26 | A | C |
| ATOM | 545 | CD2 | HIS | A | 175 | 72.023 | 17.379 | 113.203 | 1.00 | 17.16 | A | C |
| ATOM | 546 | ND1 | HIS | A | 175 | 73.354 | 18.341 | 114.644 | 1.00 | 20.15 | A | N |
| ATOM | 547 | GE1 | HIS | A | 175 | 72.734 | 19.302 | 113.982 | 1.00 | 18.26 | A | C |
| ATOM | 548 | NE2 | HIS | A | 175 | 71.922 | 18.744 | 113.102 | 1.00 | 15.77 | A | N |
| ATOM | 549 | C | HIS | A | 175 | 74.392 | 14.949 | 112.571 | 1.00 | 30.79 | A | C |
| ATOM | 550 | O | HIS | A | 175 | 73.257 | 14.536 | 112.317 | 1.00 | 32.24 | A | C |
| ATOM | 551 | N | GLY | A | 176 | 75.341 | 15.042 | 111.647 | 1.00 | 32.67 | A | N |
| ATOM | 552 | CA | GLY | A | 176 | 75.048 | 14.601 | 110.297 | 1.00 | 34.93 | A | C |
| ATOM | 553 | C | GLY | A | 176 | 75.221 | 15.634 | 109.213 | 1.00 | 36.14 | A | C |
| ATOM | 554 | O | GLY | A | 176 | 76.066 | 15.481 | 108.340 | 1.00 | 37.19 | A | O |
| ATOM | 555 | N | ASP | A | 177 | 74.399 | 16.674 | 109.240 | 1.00 | 37.27 | A | N |
| ATOM | 556 | CA | ASP | A | 177 | 74.512 | 17.721 | 108.242 | 1.00 | 35.71 | A | C |
| ATOM | 557 | CB | ASP | A | 177 | 73.267 | 18.600 | 108.231 | 1.00 | 34.06 | A | C |
| ATOM | 558 | CG | ASP | A | 177 | 72.828 | 19.006 | 109.617 | 1.00 | 32.01 | A | C |
| ATOM | 559 | OD1 | ASP | A | 177 | 73.684 | 19.394 | 110.439 | 1.00 | 28.22 | A | O |
| ATOM | 560 | OD2 | ASP | A | 177 | 71.613 | 18.942 | 109.872 | 1.00 | 33.33 | A | O |
| ATOM | 561 | C | ASP | A | 177 | 75.734 | 18.551 | 108.587 | 1.00 | 36.61 | A | C |
| ATOM | 562 | O | ASP | A | 177 | 76.570 | 18.132 | 109.389 | 1.00 | 40.71 | A | O |
| ATOM | 563 | N | GLY | A | 178 | 75.845 | 19.730 | 107.996 | 1.00 | 34.57 | A | N |
| ATOM | 564 | CA | GLY | A | 178 | 77.008 | 20.546 | 108.278 | 1.00 | 36.70 | A | C |
| ATOM | 565 | C | GLY | A | 178 | 76.753 | 21.721 | 109.194 | 1.00 | 36.80 | A | C |
| ATOM | 566 | O | GLY | A | 178 | 77.520 | 22.684 | 109.184 | 1.00 | 37.18 | A | O |
| ATOM | 567 | N | TYR | A | 179 | 75.688 | 21.649 | 109.991 | 1.00 | 37.02 | A | N |
| ATOM | 568 | CA | TYR | A | 179 | 75.344 | 22.731 | 110.908 | 1.00 | 34.28 | A | C |
| ATOM | 569 | CB | TYR | A | 179 | 74.078 | 23.436 | 110.425 | 1.00 | 36.20 | A | C |
| ATOM | 570 | CG | TYR | A | 179 | 74.181 | 23.932 | 109.003 | 1.00 | 42.47 | A | C |
| ATOM | 571 | CD1 | TYR | A | 179 | 73.949 | 23.076 | 107.927 | 1.00 | 45.68 | A | C |
| ATOM | 572 | CE1 | TYR | A | 179 | 74.065 | 23.524 | 106.609 | 1.00 | 46.33 | A | C |
| ATOM | 573 | CD2 | TYR | A | 179 | 74.536 | 25.253 | 108.728 | 1.00 | 44.79 | A | C |
| ATOM | 574 | CE2 | TYR | A | 179 | 74.658 | 25.712 | 107.415 | 1.00 | 45.00 | A | C |
| ATOM | 575 | CZ | TYR | A | 179 | 74.420 | 24.843 | 106.362 | 1.00 | 47.34 | A | C |
| ATOM | 576 | OH | TYR | A | 179 | 74.528 | 25.293 | 105.062 | 1.00 | 48.94 | A | O |
| ATOM | 577 | C | TYR | A | 179 | 75.153 | 22.235 | 112.335 | 1.00 | 31.65 | A | C |
| ATOM | 578 | O | TYR | A | 179 | 74.033 | 22.165 | 112.838 | 1.00 | 29.30 | A | O |
| ATOM | 579 | N | PRO | A | 180 | 76.260 | 21.895 | 113.014 | 1.00 | 31.79 | A | N |
| ATOM | 580 | CD | PRO | A | 180 | 77.653 | 22.113 | 112.589 | 1.00 | 30.47 | A | C |
| ATOM | 581 | CA | PRO | A | 180 | 76.212 | 21.401 | 114.394 | 1.00 | 30.83 | A | C |
| ATOM | 582 | CB | PRO | A | 180 | 77.685 | 21.194 | 114.739 | 1.00 | 31.29 | A | C |
| ATOM | 583 | CG | PRO | A | 180 | 78.370 | 22.238 | 113.916 | 1.00 | 31.47 | A | C |
| ATOM | 584 | C | PRO | A | 180 | 75.532 | 22.381 | 115.335 | 1.00 | 28.21 | A | C |
| ATOM | 585 | O | PRO | A | 180 | 75.505 | 23.579 | 115.073 | 1.00 | 28.60 | A | O |
| ATOM | 586 | N | PHE | A | 181 | 74.971 | 21.866 | 116.422 | 1.00 | 25.93 | A | N |
| ATOM | 587 | CA | PHE | A | 181 | 74.306 | 22.721 | 117.388 | 1.00 | 26.27 | A | C |
| ATOM | 588 | CB | PHE | A | 181 | 73.238 | 21.937 | 118.158 | 1.00 | 23.67 | A | C |
| ATOM | 589 | CG | PHE | A | 181 | 72.012 | 21.625 | 117.346 | 1.00 | 21.97 | A | C |
| ATOM | 590 | CD1 | PHE | A | 181 | 71.262 | 22.649 | 116.778 | 1.00 | 22.39 | A | C |
| ATOM | 591 | CD2 | PHE | A | 181 | 71.590 | 20.309 | 117.165 | 1.00 | 21.41 | A | C |
| ATOM | 592 | CE1 | PHE | A | 181 | 70.102 | 22.369 | 116.040 | 1.00 | 22.23 | A | C |
| ATOM | 593 | CE2 | PHE | A | 181 | 70.432 | 20.019 | 116.430 | 1.00 | 20.84 | A | C |
| ATOM | 594 | CZ | PHE | A | 181 | 69.688 | 21.052 | 115.868 | 1.00 | 19.15 | A | C |
| ATOM | 595 | C | PHE | A | 181 | 75.351 | 23.306 | 118.335 | 1.00 | 28.41 | A | C |
| ATOM | 596 | O | PHE | A | 181 | 76.498 | 22.863 | 118.354 | 1.00 | 27.60 | A | O |
| ATOM | 597 | N | ASP | A | 182 | 74.943 | 24.290 | 119.127 | 1.00 | 28.53 | A | N |
| ATOM | 598 | CA | ASP | A | 182 | 75.848 | 24.979 | 120.031 | 1.00 | 29.79 | A | C |
| ATOM | 599 | CB | ASP | A | 182 | 75.723 | 26.478 | 119.769 | 1.00 | 28.81 | A | C |
| ATOM | 600 | CG | ASP | A | 182 | 74.284 | 26.937 | 119.799 | 1.00 | 29.97 | A | C |
| ATOM | 601 | OD1 | ASP | A | 182 | 73.464 | 26.193 | 120.358 | 1.00 | 30.97 | A | O |
| ATOM | 602 | OD2 | ASP | A | 182 | 73.962 | 28.025 | 119.284 | 1.00 | 31.92 | A | O |
| ATOM | 603 | C | ASP | A | 182 | 75.677 | 24.718 | 121.535 | 1.00 | 31.81 | A | C |
| ATOM | 604 | O | ASP | A | 182 | 75.941 | 25.608 | 122.346 | 1.00 | 34.01 | A | O |
| ATOM | 605 | N | GLY | A | 183 | 75.249 | 23.520 | 121.921 | 1.00 | 31.93 | A | N |
| ATOM | 606 | CA | GLY | A | 183 | 75.085 | 23.236 | 123.341 | 1.00 | 31.05 | A | C |
| ATOM | 607 | C | GLY | A | 183 | 73.943 | 24.014 | 123.976 | 1.00 | 32.23 | A | C |
| ATOM | 608 | O | GLY | A | 183 | 73.094 | 24.524 | 123.276 | 1.00 | 32.15 | A | O |
| ATOM | 609 | N | LYS | A | 184 | 73.923 | 24.122 | 125.299 | 1.00 | 32.11 | A | N |
| ATOM | 610 | CA | LYS | A | 184 | 72.851 | 24.832 | 126.002 | 1.00 | 33.02 | A | C |
| ATOM | 611 | CB | LYS | A | 184 | 73.209 | 24.984 | 127.484 | 1.00 | 37.17 | A | C |
| ATOM | 612 | CG | LYS | A | 184 | 72.020 | 25.232 | 128.396 | 1.00 | 41.04 | A | C |
| ATOM | 613 | CD | LYS | A | 184 | 71.101 | 24.017 | 128.419 | 1.00 | 44.80 | A | C |
| ATOM | 614 | CE | LYS | A | 184 | 69.925 | 24.219 | 129.353 | 1.00 | 45.32 | A | C |
| ATOM | 615 | NZ | LYS | A | 184 | 70.371 | 24.435 | 130.752 | 1.00 | 48.28 | A | N |
| ATOM | 616 | C | LYS | A | 184 | 72.539 | 26.207 | 125.424 | 1.00 | 31.99 | A | C |
| ATOM | 617 | O | LYS | A | 184 | 73.445 | 26.975 | 125.118 | 1.00 | 34.08 | A | O |
| ATOM | 618 | N | ASP | A | 185 | 71.250 | 26.504 | 125.278 | 1.00 | 30.60 | A | N |
| ATOM | 619 | CA | ASP | A | 185 | 70.777 | 27.787 | 124.756 | 1.00 | 29.01 | A | C |
| ATOM | 620 | CB | ASP | A | 185 | 71.068 | 28.895 | 125.763 | 1.00 | 33.03 | A | C |
| ATOM | 621 | CG | ASP | A | 185 | 70.304 | 28.712 | 127.046 | 1.00 | 36.57 | A | C |
| ATOM | 622 | OD1 | ASP | A | 185 | 69.058 | 28.796 | 127.011 | 1.00 | 39.21 | A | O |
| ATOM | 623 | OD2 | ASP | A | 185 | 70.947 | 28.470 | 128.090 | 1.00 | 40.55 | A | O |
| ATOM | 624 | C | ASP | A | 185 | 71.311 | 28.198 | 123.394 | 1.00 | 26.92 | A | C |
| ATOM | 625 | O | ASP | A | 185 | 71.999 | 27.438 | 122.727 | 1.00 | 25.74 | A | O |
| ATOM | 626 | N | GLY | A | 186 | 70.992 | 29.422 | 122.990 | 1.00 | 24.58 | A | N |
| ATOM | 627 | CA | GLY | A | 186 | 71.431 | 29.904 | 121.695 | 1.00 | 26.41 | A | C |
| ATOM | 628 | C | GLY | A | 186 | 70.472 | 29.395 | 120.637 | 1.00 | 26.21 | A | C |
| ATOM | 629 | O | GLY | A | 186 | 69.280 | 29.693 | 120.691 | 1.00 | 27.15 | A | O |
| ATOM | 630 | N | LEU | A | 187 | 70.987 | 28.625 | 119.684 | 1.00 | 26.00 | A | N |
| ATOM | 631 | CA | LEU | A | 187 | 70.166 | 28.058 | 118.622 | 1.00 | 25.00 | A | C |
| ATOM | 632 | CB | LEU | A | 187 | 71.057 | 27.560 | 117.483 | 1.00 | 25.51 | A | C |
| ATOM | 633 | CG | LEU | A | 187 | 70.323 | 27.068 | 116.233 | 1.00 | 31.44 | A | C |
| ATOM | 634 | CD1 | LEU | A | 187 | 69.618 | 28.248 | 115.564 | 1.00 | 27.44 | A | C |
| ATOM | 635 | CD2 | LEU | A | 187 | 71.313 | 26.409 | 115.271 | 1.00 | 32.87 | A | C |
| ATOM | 636 | C | LEU | A | 187 | 69.394 | 26.885 | 119.220 | 1.00 | 24.47 | A | C |
| ATOM | 637 | O | LEU | A | 187 | 69.996 | 25.979 | 119.775 | 1.00 | 23.75 | A | O |
| ATOM | 638 | N | LEU | A | 188 | 68.071 | 26.881 | 119.097 | 1.00 | 22.38 | A | N |
| ATOM | 639 | CA | LEU | A | 188 | 67.272 | 25.800 | 119.673 | 1.00 | 20.10 | A | C |
| ATOM | 640 | CB | LEU | A | 188 | 65.930 | 26.349 | 120.158 | 1.00 | 17.21 | A | C |
| ATOM | 641 | CG | LEU | A | 188 | 65.981 | 27.580 | 121.077 | 1.00 | 19.72 | A | C |
| ATOM | 642 | CD1 | LEU | A | 188 | 64.565 | 27.997 | 121.447 | 1.00 | 14.44 | A | C |
| ATOM | 643 | CD2 | LEU | A | 188 | 66.791 | 27.275 | 122.329 | 1.00 | 16.62 | A | C |
| ATOM | 644 | C | LEU | A | 188 | 67.026 | 24.638 | 118.712 | 1.00 | 21.08 | A | C |
| ATOM | 645 | O | LEU | A | 188 | 66.907 | 23.477 | 119.129 | 1.00 | 18.95 | A | O |
| ATOM | 646 | N | ALA | A | 189 | 66.956 | 24.958 | 117.426 | 1.00 | 19.08 | A | N |
| ATOM | 647 | CA | ALA | A | 189 | 66.703 | 23.963 | 116.400 | 1.00 | 16.54 | A | C |
| ATOM | 648 | CB | ALA | A | 189 | 65.297 | 23.398 | 116.575 | 1.00 | 16.25 | A | C |
| ATOM | 649 | C | ALA | A | 189 | 66.824 | 24.626 | 115.039 | 1.00 | 17.08 | A | C |
| ATOM | 650 | O | ALA | A | 189 | 67.110 | 25.815 | 114.943 | 1.00 | 16.33 | A | O |
| ATOM | 651 | N | HIS | A | 190 | 66.627 | 23.842 | 113.988 | 1.00 | 16.66 | A | N |
| ATOM | 652 | CA | HIS | A | 190 | 66.656 | 24.361 | 112.636 | 1.00 | 18.31 | A | C |
| ATOM | 653 | CB | HIS | A | 190 | 68.099 | 24.705 | 112.191 | 1.00 | 18.50 | A | C |
| ATOM | 654 | CG | His | A | 190 | 69.055 | 23.550 | 112.164 | 1.00 | 17.26 | A | C |
| ATOM | 655 | CD2 | HIS | A | 190 | 68.920 | 22.281 | 111.712 | 1.00 | 20.42 | A | C |
| ATOM | 656 | ND1 | HIS | A | 190 | 70.369 | 23.671 | 112.567 | 1.00 | 18.18 | A | N |
| ATOM | 657 | CE1 | HIS | A | 190 | 71.002 | 22.531 | 112.365 | 1.00 | 17.27 | A | C |
| ATOM | 658 | NE2 | HIS | A | 190 | 70.145 | 21.671 | 111.846 | 1.00 | 18.89 | A | N |
| ATOM | 659 | C | HIS | A | 190 | 65.982 | 23.372 | 111.699 | 1.00 | 18.42 | A | C |
| ATOM | 660 | O | HIS | A | 190 | 65.985 | 22.168 | 111.956 | 1.00 | 18.06 | A | O |
| ATOM | 661 | N | ALA | A | 191 | 65.367 | 23.887 | 110.637 | 1.00 | 19.52 | A | N |
| ATOM | 662 | CA | ALA | A | 191 | 64.672 | 23.049 | 109.659 | 1.00 | 20.18 | A | C |
| ATOM | 663 | CB | ALA | A | 191 | 63.163 | 23.158 | 109.853 | 1.00 | 17.02 | A | C |
| ATOM | 664 | C | ALA | A | 191 | 65.052 | 23.460 | 108.242 | 1.00 | 2~.20 | A | C |
| ATOM | 665 | O | ALA | A | 191 | 65.547 | 24.563 | 108.026 | 1.00 | 22.82 | A | O |
| ATOM | 666 | N | PUB | A | 192 | 64.822 | 22.569 | 107.279 | 1.00 | 24.96 | A | N |
| ATOM | 667 | CA | PHE | A | 192 | 65.156 | 22.847 | 105.884 | 1.00 | 24.10 | A | C |
| ATOM | 668 | CB | PHE | A | 192 | 66.013 | 21.724 | 105.303 | 1.00 | 24.41 | A | C |
| ATOM | 669 | CG | PHE | A | 192 | 67.299 | 21.491 | 106.042 | 1.00 | 25.34 | A | C |
| ATOM | 670 | CD1 | PHE | A | 192 | 67.328 | 20.696 | 107.184 | 1.00 | 27.98 | A | C |
| ATOM | 671 | CD2 | PHE | A | 192 | 68.483 | 22.059 | 105.594 | 1.00 | 26.82 | A | C |
| ATOM | 672 | CE1 | PHE | A | 192 | 68.524 | 20.466 | 107.870 | 1.00 | 27.32 | A | C |
| ATOM | 673 | CE2 | PHE | A | 192 | 69.682 | 21.839 | 106.267 | 1.00 | 27.38 | A | C |
| ATOM | 674 | CZ | PHE | A | 192 | 69.703 | 21.039 | 107.410 | 1.00 | 28.50 | A | C |
| ATOM | 675 | C | PHE | A | 192 | 63.906 | 23.012 | 105.033 | 1.00 | 25.05 | A | C |
| ATOM | 676 | O | PHE | A | 192 | 62.891 | 22.346 | 105.270 | 1.00 | 24.67 | A | O |
| ATOM | 677 | N | PRO | A | 193 | 63.967 | 23.908 | 104.027 | 1.00 | 25.22 | A | N |
| ATOM | 678 | CD | PRO | A | 193 | 65.156 | 24.708 | 103.684 | 1.00 | 25.93 | A | C |
| ATOM | 679 | CA | PRO | A | 193 | 62.861 | 24.203 | 103.103 | 1.00 | 24.58 | A | C |
| ATOM | 680 | CB | PRO | A | 193 | 63.434 | 25.292 | 102.195 | 1.00 | 22.65 | A | C |
| ATOM | 681 | CG | PRO | A | 193 | 64.542 | 25.894 | 102.997 | 1.00 | 26.18 | A | C |
| ATOM | 682 | C | PRO | A | 193 | 62.454 | 22.970 | 102.299 | 1.00 | 24.59 | A | C |
| ATOM | 683 | O | PRO | A | 193 | 63.231 | 22.018 | 102.164 | 1.00 | 23.68 | A | O |
| ATOM | 684 | N | PRO | A | 194 | 61.233 | 22.981 | 101.745 | 1.00 | 23.30 | A | N |
| ATOM | 685 | CD | PRO | A | 194 | 60.243 | 24.072 | 101.820 | 1.00 | 20.97 | A | C |
| ATOM | 686 | CA | PRO | A | 194 | 60.718 | 21.861 | 100.949 | 1.00 | 24.06 | A | C |
| ATOM | 687 | CB | PRO | A | 194 | 59.445 | 22.441 | 100.334 | 1.00 | 22.11 | A | C |
| ATOM | 688 | CG | PRO | A | 194 | 58.968 | 23.371 | 101.408 | 1.00 | 21.80 | A | C |
| ATOM | 689 | C | PRO | A | 194 | 61.710 | 21.371 | 99.891 | 1.00 | 23.68 | A | C |
| ATOM | 690 | O | PRO | A | 194 | 62.473 | 22.147 | 99.325 | 1.00 | 21.32 | A | O |
| ATOM | 691 | N | GLY | A | 195 | 61.688 | 20.071 | 99.631 | 1.00 | 26.27 | A | N |
| ATOM | 692 | CA | GLY | A | 195 | 62.584 | 19.500 | 98.645 | 1.00 | 26.15 | A | C |
| ATOM | 693 | C | OLY | A | 195 | 62.908 | 18.059 | 98.978 | 1.00 | 28.20 | A | C |
| ATOM | 694 | O | GLY | A | 195 | 62.355 | 17.505 | 99.926 | 1.00 | 29.23 | A | O |
| ATOM | 695 | N | PRO | A | 196 | 63.801 | 17.420 | 98.211 | 1.00 | 28.08 | A | N |
| ATOM | 696 | CD | PRO | A | 196 | 64.340 | 17.902 | 96.926 | 1.00 | 26.14 | A | C |
| ATOM | 697 | CA | PRO | A | 196 | 64.188 | 16.025 | 98.445 | 1.00 | 28.94 | A | C |
| ATOM | 698 | CB | PRO | A | 196 | 64.613 | 15.563 | 97.057 | 1.00 | 27.73 | A | C |
| ATOM | 699 | CG | PRO | A | 196 | 65.288 | 16.788 | 96.526 | 1.00 | 26.49 | A | C |
| ATOM | 700 | C | PRO | A | 196 | 65.320 | 15.876 | 99.472 | 1.00 | 29.39 | A | C |
| ATOM | 701 | O | PRO | A | 196 | 65.972 | 16.855 | 99.837 | 1.00 | 31.26 | A | O |
| ATOM | 702 | N | GLY | A | 197 | 65.542 | 14.646 | 99.930 | 1.00 | 28.16 | A | N |
| ATOM | 703 | CA | GLY | A | 197 | 66.605 | 14.374 | 100.884 | 1.00 | 26.43 | A | C |
| ATOM | 704 | C | GLY | A | 197 | 66.466 | 15.066 | 102.223 | 1.00 | 25.89 | A | C |
| ATOM | 705 | O | GLY | A | 197 | 65.399 | 15.077 | 102.803 | 1.00 | 26.43 | A | O |
| ATOM | 706 | N | ILE | A | 198 | 67.548 | 15.648 | 102.719 | 1.00 | 24.59 | A | N |
| ATOM | 707 | CA | ILE | A | 198 | 67.515 | 16.336 | 104.000 | 1.00 | 27.36 | A | C |
| ATOM | 708 | CB | ILE | A | 198 | 68.929 | 16.809 | 104.385 | 1.00 | 29.59 | A | C |
| ATOM | 709 | CG2 | ILE | A | 198 | 69.376 | 17.929 | 103.447 | 1.00 | 29.40 | A | C |
| ATOM | 710 | CG1 | ILE | A | 198 | 68.946 | 17.268 | 105.840 | 1.00 | 30.60 | A | C |
| ATOM | 711 | CD1 | ILE | A | 198 | 70.323 | 17.634 | 106.334 | 1.00 | 33.20 | A | C |
| ATOM | 712 | C | ILE | A | 198 | 66.558 | 17.537 | 103.966 | 1.00 | 29.10 | A | C |
| ATOM | 713 | O | ILB | A | 198 | 66.139 | 18.048 | 105.009 | 1.00 | 28.33 | A | O |
| ATOM | 714 | N | GLN | A | 199 | 66.212 | 17.975 | 102.760 | 1.00 | 27.92 | A | N |
| ATOM | 715 | CA | OLN | A | 199 | 65.304 | 19.100 | 102.578 | 1.00 | 28.08 | A | C |
| ATOM | 716 | CB | GLN | A | 199 | 65.103 | 19.366 | 101.081 | 1.00 | 31.22 | A | C |
| ATOM | 717 | CG | GLN | A | 199 | 65.534 | 20.754 | 100.637 | 1.00 | 40.25 | A | C |
| ATOM | 718 | CD | GLN | A | 199 | 66.996 | 21.044 | 100.939 | 1.00 | 42.74 | A | C |
| ATOM | 719 | OE1 | GLN | A | 199 | 67.459 | 22.173 | 100.787 | 1.00 | 45.88 | A | O |
| ATOM | 720 | NE2 | GLN | A | 199 | 67.730 | 20.022 | 101.362 | 1.00 | 44.96 | A | N |
| ATOM | 721 | C | GLN | A | 199 | 63.967 | 18.794 | 103.241 | 1.00 | 23.32 | A | C |
| ATOM | 722 | O | GLN | A | 199 | 63.442 | 17.705 | 103.097 | 1.00 | 20.60 | A | O |
| ATOM | 723 | N | GLY | A | 200 | 63.416 | 19.759 | 103.967 | 1.00 | 23.07 | A | N |
| ATOM | 724 | CA | GLY | A | 200 | 62.147 | 19.537 | 104.637 | 1.00 | 19.23 | A | C |
| ATOM | 725 | C | GLY | A | 200 | 62.279 | 18.891 | 106.009 | 1.00 | 18.68 | A | C |
| ATOM | 726 | O | GLY | A | 200 | 61.283 | 18.719 | 106.708 | 1.00 | 22.62 | A | O |
| ATOM | 727 | N | ASP | A | 201 | 63.496 | 18.532 | 106.405 | 1.00 | 17.22 | A | N |
| ATOM | 728 | CA | ASP | A | 201 | 63.712 | 17.903 | 107.711 | 1.00 | 18.97 | A | C |
| ATOM | 729 | CB | ASP | A | 201 | 64.974 | 17.038 | 107.687 | 1.00 | 16.29 | A | C |
| ATOM | 730 | CG | ASP | A | 201 | 64.834 | 15.835 | 106.774 | 1.00 | 18.51 | A | C |
| ATOM | 731 | OD1 | ASP | A | 201 | 63.724 | 15.612 | 106.260 | 1.00 | 15.25 | A | O |
| ATOM | 732 | OD2 | ASP | A | 201 | 65.824 | 15.104 | 106.576 | 1.00 | 19.34 | A | O |
| ATOM | 733 | C | ASP | A | 201 | 63.813 | 18.920 | 108.844 | 1.00 | 19.43 | A | C |
| ATOM | 734 | O | ASP | A | 201 | 64.156 | 20.076 | 108.621 | 1.00 | 21.36 | A | O |
| ATOM | 735 | N | ALA | A | 202 | 63.513 | 18.476 | 110.062 | 1.00 | 19.70 | A | N |
| ATOM | 736 | CA | ALA | A | 202 | 63.559 | 19.341 | 111.239 | 1.00 | 18.01 | A | C |
| ATOM | 737 | CB | ALA | A | 202 | 62.142 | 19.616 | 111.729 | 1.00 | 17.65 | A | C |
| ATOM | 738 | C | ALA | A | 202 | 64.389 | 18.706 | 112.350 | 1.00 | 18.31 | A | C |
| ATOM | 739 | O | ALA | A | 202 | 64.107 | 17.588 | 112.790 | 1.00 | 17.93 | A | O |
| ATOM | 740 | N | HIS | A | 203 | 65.407 | 19.431 | 112.805 | 1.00 | 17.90 | A | N |
| ATOM | 741 | CA | HIS | A | 203 | 66.304 | 18.945 | 113.847 | 1.00 | 17.65 | A | C |
| ATOM | 742 | CB | HIS | A | 203 | 67.736 | 18.938 | 113.313 | 1.00 | 15.54 | A | C |
| ATOM | 743 | CG | HIS | A | 203 | 67.906 | 18.147 | 112.053 | 1.00 | 16.78 | A | C |
| ATOM | 744 | CD2 | HIS | A | 203 | 67.100 | 17.239 | 111.453 | 1.00 | 14.93 | A | C |
| ATOM | 745 | ND1 | HIS | A | 203 | 69.025 | 18.248 | 111.258 | 1.00 | 15.86 | A | N |
| ATOM | 746 | CE1 | HIS | A | 203 | 68.902 | 17.439 | 110.221 | 1.00 | 16.22 | A | C |
| ATOM | 747 | NE2 | HIS | A | 203 | 67.742 | 16.816 | 110.316 | 1.00 | 14.71 | A | N |
| ATOM | 748 | C | HIS | A | 203 | 66.241 | 19.782 | 115.123 | 1.00 | 17.94 | A | C |
| ATOM | 749 | O | HIS | A | 203 | 66.248 | 21.008 | 115.063 | 1.00 | 17.49 | A | O |
| ATOM | 750 | N | PHE | A | 204 | 66.193 | 19.115 | 116.275 | 1.00 | 17.60 | A | N |
| ATOM | 751 | CA | PHE | A | 204 | 66.133 | 19.814 | 117.559 | 1.00 | 17.27 | A | C |
| ATOM | 752 | CB | PHE | A | 204 | 64.884 | 19.389 | 118.342 | 1.00 | 15.27 | A | C |
| ATOM | 753 | CO | PHE | A | 204 | 63.602 | 19.576 | 117.579 | 1.00 | 15.59 | A | C |
| ATOM | 754 | CD1 | PHE | A | 204 | 63.234 | 18.678 | 116.581 | 1.00 | 15.30 | A | C |
| ATOM | 755 | CD2 | PHE | A | 204 | 62.790 | 20.683 | 117.816 | 1.00 | 15.08 | A | C |
| ATOM | 756 | CE1 | PHR | A | 204 | 62.075 | 18.882 | 115.826 | 1.00 | 17.01 | A | C |
| ATOM | 757 | CE2 | PHE | A | 204 | 61.636 | 20.899 | 117.071 | 1.00 | 14.70 | A | C |
| ATOM | 758 | CZ | PHE | A | 204 | 61.275 | 19.998 | 116.073 | 1.00 | 16.71 | A | C |
| ATOM | 759 | C | PHE | A | 204 | 67.378 | 19.555 | 118.402 | 1.00 | 17.45 | A | C |
| ATOM | 760 | O | PHE | A | 204 | 67.834 | 18.414 | 118.510 | 1.00 | 15.59 | A | O |
| ATOM | 761 | N | ASP | A | 205 | 67.928 | 20.619 | 118.987 | 1.00 | 16.31 | A | N |
| ATOM | 762 | CA | ASP | A | 205 | 69.109 | 20.503 | 119.837 | 1.00 | 16.90 | A | C |
| ATOM | 763 | CB | ASP | A | 205 | 69.729 | 21.876 | 120.082 | 1.00 | 17.51 | A | C |
| ATOM | 764 | CG | ASP | A | 205 | 71.037 | 21.788 | 120.840 | 1.00 | 18.97 | A | C |
| ATOM | 765 | OD1 | ASP | A | 205 | 71.305 | 20.732 | 121.441 | 1.00 | 18.91 | A | O |
| ATOM | 766 | OD2 | ASP | A | 205 | 71.798 | 22.767 | 120.836 | 1.00 | 18.05 | A | O |
| ATOM | 767 | C | ASP | A | 205 | 68.730 | 19.883 | 121.185 | 1.00 | 17.81 | A | C |
| ATOM | 768 | O | ASP | A | 205 | 68.024 | 20.498 | 121.988 | 1.00 | 18.19 | A | O |
| ATOM | 769 | N | ASP | A | 206 | 69.208 | 18.670 | 121.436 | 1.00 | 19.02 | A | N |
| ATOM | 770 | CA | ASP | A | 206 | 68.880 | 17.994 | 122.676 | 1.00 | 20.78 | A | C |
| ATOM | 771 | CB | ASP | A | 206 | 68.881 | 16.473 | 122.468 | 1.00 | 22.45 | A | C |
| ATOM | 772 | CG | ASP | A | 206 | 68.015 | 15.737 | 123.497 | 1.00 | 25.21 | A | C |
| ATOM | 773 | OD1 | ASP | A | 206 | 67.013 | 16.317 | 123.968 | 1.00 | 26.38 | A | O |
| ATOM | 774 | OD2 | ASP | A | 206 | 68.321 | 14.574 | 123.822 | 1.00 | 24.25 | A | O |
| ATOM | 775 | C | ASP | A | 206 | 69.781 | 18.386 | 123.847 | 1.00 | 21.59 | A | C |
| ATOM | 776 | O | ASP | A | 206 | 69.827 | 17.692 | 124.849 | 1.00 | 23.96 | A | O |
| ATOM | 777 | N | ASP | A | 207 | 70.513 | 19.488 | 123.715 | 1.00 | 23.27 | A | N |
| ATOM | 778 | CA | ASP | A | 207 | 71.331 | 19.969 | 124.823 | 1.00 | 22.12 | A | C |
| ATOM | 779 | CB | ASP | A | 207 | 72.605 | 20.664 | 124.342 | 1.00 | 23.08 | A | C |
| ATOM | 780 | CG | ASP | A | 207 | 73.809 | 19.738 | 124.348 | 1.00 | 24.02 | A | C |
| ATOM | 781 | OD1 | ASP | A | 207 | 73.806 | 18.762 | 125.124 | 1.00 | 26.09 | A | O |
| ATOM | 782 | OD2 | ASP | A | 207 | 74.766 | 19.989 | 123.591 | 1.00 | 23.97 | A | O |
| ATOM | 783 | C | ASP | A | 207 | 70.458 | 20.966 | 125.570 | 1.00 | 21.36 | A | C |
| ATOM | 784 | O | ASP | A | 207 | 70.817 | 21.459 | 126.634 | 1.00 | 22.22 | A | O |
| ATOM | 785 | N | GLU | A | 208 | 69.303 | 21.255 | 124.982 | 1.00 | 20.40 | A | N |
| ATOM | 786 | CA | GLU | A | 208 | 68.341 | 22.172 | 125.567 | 1.00 | 21.00 | A | C |
| ATOM | 787 | GB | GLU | A | 208 | 67.477 | 22.810 | 124.475 | 1.00 | 22.83 | A | C |
| ATOM | 788 | CG | GLU | A | 208 | 68.262 | 23.464 | 123.363 | 1.00 | 27.27 | A | C |
| ATOM | 789 | CD | GLU | A | 208 | 68.931 | 24.732 | 123.805 | 1.00 | 29.69 | A | C |
| ATOM | 790 | OE1 | GLU | A | 208 | 69.277 | 24.846 | 124.996 | 1.00 | 36.53 | A | O |
| ATOM | 791 | OE2 | GLU | A | 208 | 69.119 | 25.620 | 122.959 | 1.00 | 34.99 | A | O |
| ATOM | 792 | C | GLU | A | 208 | 67.446 | 21.345 | 126.464 | 1.00 | 19.62 | A | C |
| ATOM | 793 | O | GLU | A | 208 | 67.323 | 20.140 | 126.271 | 1.00 | 18.21 | A | O |
| ATOM | 794 | N | LEU | A | 209 | 66.831 | 21.990 | 127.447 | 1.00 | 18.09 | A | N |
| ATOM | 795 | CA | LEU | A | 209 | 65.898 | 21.311 | 128.331 | 1.00 | 17.54 | A | C |
| ATOM | 796 | GB | LBU | A | 209 | 65.887 | 21.982 | 129.710 | 1.00 | 15.57 | A | C |
| ATOM | 797 | CG | LEU | A | 209 | 64.850 | 21.473 | 130.714 | 1.00 | 19.72 | A | C |
| ATOM | 798 | CD1 | LEU | A | 209 | 65.072 | 20.004 | 130.986 | 1.00 | 18.15 | A | C |
| ATOM | 799 | CD2 | LEU | A | 209 | 64.947 | 22.273 | 132.008 | 1.00 | 18.92 | A | C |
| ATOM | 800 | C | LEU | A | 209 | 64.529 | 21.445 | 127.653 | 1.00 | 16.85 | A | C |
| ATOM | 801 | O | LEU | A | 209 | 64.001 | 22.549 | 127.519 | 1.00 | 18.96 | A | O |
| ATOM | 802 | N | TRP | A | 210 | 63.974 | 20.328 | 127.197 | 1.00 | 15.69 | A | N |
| ATOM | 803 | CA | TRP | A | 210 | 62.678 | 20.347 | 126.531 | 1.00 | 14.68 | A | C |
| ATOM | 804 | CB | TRP | A | 210 | 62.628 | 19.319 | 125.384 | 1.00 | 12.69 | A | C |
| ATOM | 805 | CG | TRP | A | 210 | 63.520 | 19.686 | 124.252 | 1.00 | 14.19 | A | C |
| ATOM | 806 | CD2 | TRP | A | 210 | 63.396 | 20.834 | 123.402 | 1.00 | 14.74 | A | C |
| ATOM | 807 | CE2 | TRP | A | 210 | 64.531 | 20.855 | 122.562 | 1.00 | 13.82 | A | C |
| ATOM | 808 | CE3 | TRP | A | 210 | 62.438 | 21.850 | 123.273 | 1.00 | 13.00 | A | C |
| ATOM | 809 | CD1 | TRP | A | 210 | 64.688 | 19.072 | 123.893 | 1.00 | 15.08 | A | C |
| ATOM | 810 | NE1 | TRP | A | 210 | 65.302 | 19.770 | 122.885 | 1.00 | 15.30 | A | N |
| ATOM | 811 | CZ2 | TRP | A | 210 | 64.736 | 21.854 | 121.605 | 1.00 | 12.58 | A | C |
| ATOM | 812 | CZ3 | TRP | A | 210 | 62.646 | 22.845 | 122.319 | 1.00 | 11.89 | A | C |
| ATOM | 813 | CH2 | TRP | A | 210 | 63.787 | 22.837 | 121.499 | 1.00 | 10.54 | A | C |
| ATOM | 814 | C | TRP | A | 210 | 61.549 | 20.080 | 127.503 | 1.00 | 14.10 | A | C |
| ATOM | 815 | O | TRP | A | 210 | 61.614 | 19.147 | 128.301 | 1.00 | 12.40 | A | O |
| ATOM | 816 | N | SER | A | 211 | 60.504 | 20.897 | 127.406 | 1.00 | 13.94 | A | N |
| ATOM | 817 | CA | SER | A | 211 | 59.347 | 20.791 | 128.272 | 1.00 | 13.15 | A | C |
| ATOM | 818 | GB | SER | A | 211 | 59.568 | 21.682 | 129.492 | 1.00 | 14.15 | A | C |
| ATOM | 819 | OG | SER | A | 211 | 58.635 | 21.392 | 130.505 | 1.00 | 17.46 | A | O |
| ATOM | 820 | C | SER | A | 211 | 58.102 | 21.243 | 127.505 | 1.00 | 15.84 | A | C |
| ATOM | 821 | O | SER | A | 211 | 58.080 | 21.195 | 126.280 | 1.00 | 17.08 | A | O |
| ATOM | 822 | N | LEU | A | 212 | 57.068 | 21.667 | 128.233 | 1.00 | 15.21 | A | N |
| ATOM | 823 | CA | LEU | A | 212 | 55.819 | 22.160 | 127.645 | 1.00 | 13.96 | A | C |
| ATOM | 824 | CB | LEU | A | 212 | 54.836 | 21.018 | 127.391 | 1.00 | 10.99 | A | C |
| ATOM | 825 | CO | LEU | A | 212 | 55.168 | 19.921 | 126.378 | 1.00 | 11.66 | A | C |
| ATOM | 826 | CD1 | LEU | A | 212 | 54.139 | 18.817 | 126.510 | 1.00 | 8.67 | A | C |
| ATOM | 827 | CD2 | LEU | A | 212 | 55.202 | 20.494 | 124.967 | 1.00 | 7.27 | A | C |
| ATOM | 828 | C | LEU | A | 212 | 55.180 | 23.125 | 128.635 | 1.00 | 17.00 | A | C |
| ATOM | 829 | O | LEU | A | 212 | 55.367 | 22.992 | 129.837 | 1.00 | 19.75 | A | O |
| ATOM | 830 | N | GLY | A | 213 | 54.421 | 24.088 | 128.132 | 1.00 | 19.26 | A | N |
| ATOM | 831 | CA | GLY | A | 213 | 53.748 | 25.037 | 129.003 | 1.00 | 20.32 | A | C |
| ATOM | 832 | C | GLY | A | 213 | 54.639 | 26.040 | 129.713 | 1.00 | 19.73 | A | C |
| ATOM | 833 | O | GLY | A | 213 | 55.759 | 26.294 | 129.285 | 1.00 | 20.34 | A | O |
| ATOM | 834 | N | LYS | A | 214 | 54.123 | 26.618 | 130.795 | 1.00 | 20.81 | A | N |
| ATOM | 835 | CA | LYS | A | 214 | 54.861 | 27.597 | 131.588 | 1.00 | 21.44 | A | C |
| ATOM | 836 | CB | LYS | A | 214 | 53.909 | 28.339 | 132.537 | 1.00 | 23.26 | A | C |
| ATOM | 837 | CG | LYS | A | 214 | 52.925 | 29.266 | 131.838 | 1.00 | 25.72 | A | C |
| ATOM | 838 | CD | LYS | A | 214 | 51.935 | 29.898 | 132.805 | 1.00 | 27.35 | A | C |
| ATOM | 839 | CE | LYS | A | 214 | 51.002 | 28.859 | 133.402 | 1.00 | 30.75 | A | C |
| ATOM | 840 | NZ | LYS | A | 214 | 49.866 | 29.485 | 134.138 | 1.00 | 35.19 | A | N |
| ATOM | 841 | C | LYS | A | 214 | 55.917 | 26.863 | 132.398 | 1.00 | 21.38 | A | C |
| ATOM | 842 | O | LYS | A | 214 | 55.743 | 25.688 | 132.729 | 1.00 | 22.09 | A | O |
| ATOM | 843 | N | GLY | A | 215 | 57.008 | 27.548 | 132.721 | 1.00 | 19.58 | A | N |
| ATOM | 844 | CA | GLY | A | 215 | 58.061 | 26.911 | 133.491 | 1.00 | 20.65 | A | C |
| ATOM | 845 | C | GLY | A | 215 | 59.403 | 27.007 | 132.798 | 1.00 | 20.80 | A | C |
| ATOM | 846 | O | GLY | A | 215 | 59.496 | 27.528 | 131.689 | 1.00 | 20.47 | A | O |
| ATOM | 847 | N | GLN | A | 391 | 60.449 | 26.500 | 133.439 | 1.00 | 21.31 | A | N |
| ATOM | 848 | CA | GLN | A | 391 | 61.779 | 26.564 | 132.848 | 1.00 | 22.25 | A | C |
| ATOM | 849 | CB | GLN | A | 391 | 62.851 | 26.279 | 133.917 | 1.00 | 26.72 | A | C |
| ATOM | 850 | CG | GLN | A | 391 | 63.213 | 24.832 | 134.110 | 1.00 | 34.22 | A | C |
| ATOM | 851 | CD | GLN | A | 391 | 62.000 | 23.953 | 134.238 | 1.00 | 41.18 | A | C |
| ATOM | 852 | OE1 | OLN | A | 391 | 61.117 | 24.211 | 135.054 | 1.00 | 48.75 | A | O |
| ATOM | 853 | NE2 | GLN | A | 391 | 61.944 | 22.903 | 133.427 | 1.00 | 46.74 | A | N |
| ATOM | 854 | C | GLN | A | 391 | 61.886 | 25.585 | 131.676 | 1.00 | 20.85 | A | C |
| ATOM | 855 | O | GLN | A | 391 | 61.097 | 24.654 | 131.561 | 1.00 | 20.10 | A | O |
| ATOM | 856 | N | GLY | A | 392 | 62.866 | 25.794 | 130.808 | 1.00 | 19.12 | A | N |
| ATOM | 857 | CA | GLY | A | 392 | 63.005 | 24.931 | 129.653 | 1.00 | 20.95 | A | C |
| ATOM | 858 | C | GLY | A | 392 | 62.266 | 25.565 | 128.487 | 1.00 | 19.62 | A | C |
| ATOM | 859 | O | GLY | A | 392 | 61.669 | 26.627 | 128.637 | 1.00 | 17.25 | A | O |
| ATOM | 860 | N | TYR | A | 393 | 62.301 | 24.921 | 127.328 | 1.00 | 19.08 | A | N |
| ATOM | 861 | CA | TYR | A | 393 | 61.637 | 25.454 | 126.141 | 1.00 | 17.64 | A | C |
| ATOM | 862 | CB | TYR | A | 393 | 62.646 | 25.560 | 125.000 | 1.00 | 18.65 | A | C |
| ATOM | 863 | CO | TYR | A | 393 | 63.773 | 26.534 | 125.256 | 1.00 | 18.22 | A | C |
| ATOM | 864 | CD1 | TYR | A | 393 | 63.586 | 27.905 | 125.079 | 1.00 | 19.12 | A | C |
| ATOM | 865 | CE1 | TYR | A | 393 | 64.626 | 28.807 | 125.291 | 1.00 | 20.27 | A | C |
| ATOM | 866 | CD2 | TYR | A | 393 | 65.036 | 26.084 | 125.660 | 1.00 | 18.43 | A | C |
| ATOM | 867 | CE2 | TYR | A | 393 | 66.091 | 26.980 | 125.878 | 1.00 | 18.43 | A | C |
| ATOM | 868 | CZ | TYR | A | 393 | 65.879 | 28.339 | 125.691 | 1.00 | 20.99 | A | C |
| ATOM | 869 | OH | TYR | A | 393 | 66.911 | 29.229 | 125.892 | 1.00 | 18.81 | A | O |
| ATOM | 870 | C | TYR | A | 393 | 60.474 | 24.563 | 125.714 | 1.00 | 16.46 | A | C |
| ATOM | 871 | O | TYR | A | 393 | 60.552 | 23.343 | 125.812 | 1.00 | 18.48 | A | O |
| ATOM | 872 | N | SER | A | 394 | 59.393 | 25.165 | 125.238 | 1.00 | 16.19 | A | N |
| ATOM | 873 | CA | SER | A | 394 | 58.248 | 24.376 | 124.799 | 1.00 | 16.88 | A | C |
| ATOM | 874 | CB | SER | A | 394 | 57.026 | 25.268 | 124.581 | 1.00 | 15.95 | A | C |
| ATOM | 875 | OG | SER | A | 394 | 55.900 | 24.491 | 124.195 | 1.00 | 16.81 | A | O |
| ATOM | 876 | C | SER | A | 394 | 58.555 | 23.638 | 123.501 | 1.00 | 16.05 | A | C |
| ATOM | 877 | O | SER | A | 394 | 58.781 | 24.262 | 122.468 | 1.00 | 17.59 | A | O |
| ATOM | 878 | N | LEU | A | 395 | 58.563 | 22.311 | 123.557 | 1.00 | 13.28 | A | N |
| ATOM | 879 | CA | LEU | A | 395 | 58.825 | 21.508 | 122.373 | 1.00 | 13.52 | A | C |
| ATOM | 880 | CB | LEU | A | 395 | 58.874 | 20.022 | 122.734 | 1.00 | 9.81 | A | C |
| ATOM | 881 | CG | LEU | A | 395 | 59.158 | 19.078 | 121.559 | 1.00 | 10.42 | A | C |
| ATOM | 882 | CD1 | LEU | A | 395 | 60.518 | 19.417 | 120.974 | 1.00 | 8.66 | A | C |
| ATOM | 883 | CD2 | LEU | A | 395 | 59.117 | 17.618 | 122.012 | 1.00 | 7.01 | A | C |
| ATOM | 884 | C | LEU | A | 395 | 57.720 | 21.758 | 121.345 | 1.00 | 13.87 | A | C |
| ATOM | 885 | O | LEU | A | 395 | 57.979 | 21.792 | 120.149 | 1.00 | 15.88 | A | O |
| ATOM | 886 | N | PHE | A | 396 | 56.495 | 21.936 | 121.836 | 1.00 | 13.30 | A | N |
| ATOM | 887 | CA | PHE | A | 396 | 55.319 | 22.195 | 121.010 | 1.00 | 13.78 | A | C |
| ATOM | 888 | CB | PHE | A | 396 | 54.069 | 22.270 | 121.907 | 1.00 | 14.03 | A | C |
| ATOM | 889 | CG | PHE | A | 396 | 52.850 | 22.840 | 121.225 | 1.00 | 15.47 | A | C |
| ATOM | 890 | CD1 | PHE | A | 396 | 52.243 | 22.168 | 120.167 | 1.00 | 16.74 | A | C |
| ATOM | 891 | CD2 | PHE | A | 396 | 52.322 | 24.064 | 121.632 | 1.00 | 16.39 | A | C |
| ATOM | 892 | CE1 | PHE | A | 396 | 51.128 | 22.706 | 119.518 | 1.00 | 15.98 | A | C |
| ATOM | 893 | CE2 | PHE | A | 396 | 51.211 | 24.611 | 120.993 | 1.00 | 17.97 | A | C |
| ATOM | 894 | CZ | PHE | A | 396 | 50.611 | 23.929 | 119.929 | 1.00 | 16.67 | A | C |
| ATOM | 895 | C | PHE | A | 396 | 55.440 | 23.476 | 120.178 | 1.00 | 15.62 | A | C |
| ATOM | 896 | O | PHE | A | 396 | 55.242 | 23.455 | 118.958 | 1.00 | 15.38 | A | O |
| ATOM | 897 | N | LEU | A | 397 | 55.756 | 24.587 | 120.837 | 1.00 | 14.66 | A | N |
| ATOM | 898 | CA | LEU | A | 397 | 55.892 | 25.861 | 120.148 | 1.00 | 14.57 | A | C |
| ATOM | 899 | CB | LEU | A | 397 | 55.998 | 27.009 | 121.155 | 1.00 | 16.23 | A | C |
| ATOM | 900 | CG | LEU | A | 397 | 54.739 | 27.366 | 121.951 | 1.00 | 19.61 | A | C |
| ATOM | 901 | CD1 | LEU | A | 397 | 55.033 | 28.532 | 122.908 | 1.00 | 17.04 | A | C |
| ATOM | 902 | CD2 | LEU | A | 397 | 53.615 | 27.734 | 120.982 | 1.00 | 18.70 | A | C |
| ATOM | 903 | C | LEU | A | 397 | 57.084 | 25.912 | 119.202 | 1.00 | 16.31 | A | C |
| ATOM | 904 | O | LEU | A | 397 | 57.002 | 26.511 | 118.132 | 1.00 | 18.67 | A | O |
| ATOM | 905 | N | VAL | A | 398 | 58.198 | 25.299 | 119.591 | 1.00 | 17.18 | A | N |
| ATOM | 906 | CA | VAL | A | 398 | 59.379 | 25.309 | 118.738 | 1.00 | 16.87 | A | C |
| ATOM | 907 | GB | VAL | A | 398 | 60.643 | 24.855 | 119.513 | 1.00 | 15.03 | A | C |
| ATOM | 908 | CG1 | VAL | A | 398 | 61.838 | 24.750 | 118.562 | 1.00 | 7.55 | A | C |
| ATOM | 909 | CG2 | VAL | A | 398 | 60.953 | 25.867 | 120.636 | 1.00 | 13.62 | A | C |
| ATOM | 910 | C | VAL | A | 398 | 59.157 | 24.410 | 117.524 | 1.00 | 17.35 | A | C |
| ATOM | 911 | O | VAL | A | 398 | 59.610 | 24.715 | 116.427 | 1.00 | 16.12 | A | O |
| ATOM | 912 | N | ALA | A | 399 | 58.446 | 23.307 | 117.725 | 1.00 | 17.18 | A | N |
| ATOM | 913 | CA | ALA | A | 399 | 58.159 | 22.386 | 116.629 | 1.00 | 18.10 | A | C |
| ATOM | 914 | CB | ALA | A | 399 | 57.440 | 21.146 | 117.155 | 1.00 | 17.14 | A | C |
| ATOM | 915 | C | ALA | A | 399 | 57.296 | 23.084 | 115.583 | 1.00 | 16.31 | A | C |
| ATOM | 916 | O | ALA | A | 399 | 57.540 | 22.962 | 114.387 | 1.00 | 17.90 | A | O |
| ATOM | 917 | N | ALA | A | 400 | 56.288 | 23.814 | 116.048 | 1.00 | 16.32 | A | N |
| ATOM | 918 | CA | ALA | A | 400 | 55.385 | 24.531 | 115.161 | 1.00 | 16.53 | A | C |
| ATOM | 919 | CB | ALA | A | 400 | 54.340 | 25.283 | 115.970 | 1.00 | 11.88 | A | C |
| ATOM | 920 | C | ALA | A | 400 | 56.162 | 25.495 | 114.278 | 1.00 | 17.89 | A | C |
| ATOM | 921 | O | ALA | A | 400 | 55.859 | 25.640 | 113.101 | 1.00 | 18.21 | A | O |
| ATOM | 922 | N | HIS | A | 401 | 57.169 | 26.142 | 114.858 | 1.00 | 18.94 | A | N |
| ATOM | 923 | CA | HIS | A | 401 | 58.009 | 27.094 | 114.140 | 1.00 | 16.00 | A | C |
| ATOM | 924 | CB | HIS | A | 401 | 58.934 | 27.812 | 115.123 | 1.00 | 15.08 | A | C |
| ATOM | 925 | CG | HIS | A | 401 | 59.800 | 28.858 | 114.490 | 1.00 | 16.12 | A | C |
| ATOM | 926 | CD2 | HIS | A | 401 | 61.049 | 28.788 | 113.972 | 1.00 | 11.82 | A | C |
| ATOM | 927 | ND1 | HIS | A | 401 | 59.400 | 30.169 | 114.344 | 1.00 | 16.08 | A | N |
| ATOM | 928 | CE1 | HIS | A | 401 | 60.367 | 30.860 | 113.767 | 1.00 | 16.27 | A | C |
| ATOM | 929 | NE2 | HIS | A | 401 | 61.377 | 30.045 | 113.531 | 1.00 | 10.47 | A | N |
| ATOM | 930 | C | HIS | A | 401 | 58.849 | 26.371 | 113.091 | 1.00 | 17.48 | A | C |
| ATOM | 931 | O | HIS | A | 401 | 58.899 | 26.785 | 111.939 | 1.00 | 17.04 | A | O |
| ATOM | 932 | N | GLU | A | 402 | 59.512 | 25.292 | 113.497 | 1.00 | 16.98 | A | N |
| ATOM | 933 | CA | GLU | A | 402 | 60.346 | 24.523 | 112.583 | 1.00 | 19.94 | A | C |
| ATOM | 934 | CB | GLU | A | 402 | 61.058 | 23.374 | 113.311 | 1.00 | 20.34 | A | C |
| ATOM | 935 | CG | GLU | A | 402 | 62.029 | 23.804 | 114.410 | 1.00 | 20.68 | A | C |
| ATOM | 936 | CD | GLU | A | 402 | 62.758 | 25.100 | 114.095 | 1.00 | 17.45 | A | C |
| ATOM | 937 | CE1 | GLU | A | 402 | 63.300 | 25.234 | 112.987 | 1.00 | 18.70 | A | O |
| ATOM | 938 | CE2 | GLU | A | 402 | 62.793 | 25.984 | 114.967 | 1.00 | 17.38 | A | O |
| ATOM | 939 | C | GLU | A | 402 | 59.522 | 23.952 | 111.432 | 1.00 | 20.70 | A | C |
| ATOM | 940 | O | GLU | A | 402 | 59.961 | 23.972 | 110.285 | 1.00 | 20.70 | A | 0 |
| ATOM | 941 | N | PHE | A | 403 | 58.336 | 23.437 | 111.735 | 1.00 | 20.02 | A | N |
| ATOM | 942 | CA | PHE | A | 403 | 57.482 | 22.889 | 110.691 | 1.00 | 20.64 | A | C |
| ATOM | 943 | CB | PHE | A | 403 | 56.262 | 22.184 | 111.293 | 1.00 | 20.51 | A | C |
| ATOM | 944 | CG | PHE | A | 403 | 56.608 | 21.005 | 112.173 | 1.00 | 20.43 | A | C |
| ATOM | 945 | CD1 | PHE | A | 403 | 57.865 | 20.398 | 112.094 | 1.00 | 17.64 | A | C |
| ATOM | 946 | CD2 | PHE | A | 403 | 55.678 | 20.509 | 113.087 | 1.00 | 14.31 | A | C |
| ATOM | 947 | CE1 | PHE | A | 403 | 58.192 | 19.319 | 112.916 | 1.00 | 18.71 | A | C |
| ATOM | 948 | CE2 | PRE | A | 403 | 55.988 | 19.424 | 113.918 | 1.00 | 12.75 | A | C |
| ATOM | 949 | CZ | PHE | A | 403 | 57.243 | 18.826 | 113.838 | 1.00 | 15.90 | A | C |
| ATOM | 950 | C | PHE | A | 403 | 57.040 | 23.998 | 109.732 | 1.00 | 20.93 | A | C |
| ATOM | 951 | O | PHE | A | 403 | 56.699 | 23.728 | 108.587 | 1.00 | 20.08 | A | O |
| ATOM | 952 | N | GLY | A | 404 | 57.041 | 25.241 | 110.208 | 1.00 | 20.24 | A | N |
| ATOM | 953 | CA | GLY | A | 404 | 56.685 | 26.355 | 109.352 | 1.00 | 18.45 | A | C |
| ATOM | 954 | C | GLY | A | 404 | 57.748 | 26.442 | 108.263 | 1.00 | 19.06 | A | C |
| ATOM | 955 | O | GLY | A | 404 | 57.438 | 26.584 | 107.082 | 1.00 | 18.85 | A | O |
| ATOM | 956 | N | HIS | A | 405 | 59.011 | 26.353 | 108.666 | 1.00 | 17.32 | A | N |
| ATOM | 957 | CA | HIS | A | 405 | 60.119 | 26.382 | 107.718 | 1.00 | 18.09 | A | C |
| ATOM | 958 | CB | HIS | A | 405 | 61.460 | 26.317 | 108.446 | 1.00 | 15.13 | A | C |
| ATOM | 959 | CG | HIS | A | 405 | 61.859 | 27.600 | 109.095 | 1.00 | 19.30 | A | C |
| ATOM | 960 | CD2 | HIS | A | 405 | 62.362 | 27.859 | 110.323 | 1.00 | 15.40 | A | C |
| ATOM | 961 | ND1 | HIS | A | 405 | 61.769 | 28.816 | 108.452 | 1.00 | 18.08 | A | N |
| ATOM | 962 | CE1 | HIS | A | 405 | 62.197 | 29.769 | 109.258 | 1.00 | 17.34 | A | C |
| ATOM | 963 | NE2 | HIS | A | 405 | 62.563 | 29.214 | 110.399 | 1.00 | 18.40 | A | N |
| ATOM | 964 | C | HIS | A | 405 | 60.040 | 25.195 | 106.761 | 1.00 | 18.79 | A | C |
| ATOM | 965 | O | HIS | A | 405 | 60.171 | 25.352 | 105.554 | 1.00 | 22.20 | A | O |
| ATOM | 966 | N | ALA | A | 406 | 59.838 | 24.007 | 107.320 | 1.00 | 18.37 | A | N |
| ATOM | 967 | CA | ALA | A | 406 | 59.754 | 22.780 | 106.545 | 1.00 | 17.99 | A | C |
| ATOM | 968 | CB | ALA | A | 406 | 59.511 | 21.606 | 107.484 | 1.00 | 15.77 | A | C |
| ATOM | 969 | C | ALA | A | 406 | 58.665 | 22.838 | 105.468 | 1.00 | 17.88 | A | C |
| ATOM | 970 | O | ALA | A | 406 | 58.673 | 22.045 | 104.535 | 1.00 | 15.94 | A | O |
| ATOM | 971 | N | LEU | A | 407 | 57.730 | 23.773 | 105.612 | 1.00 | 20.32 | A | N |
| ATOM | 972 | CA | LEU | A | 407 | 56.646 | 23.935 | 104.647 | 1.00 | 22.45 | A | C |
| ATOM | 973 | CB | LEU | A | 407 | 55.307 | 24.158 | 105.367 | 1.00 | 21.52 | A | C |
| ATOM | 974 | CG | LEU | A | 407 | 54.789 | 23.030 | 106.266 | 1.00 | 22.77 | A | C |
| ATOM | 975 | CD1 | LEU | A | 407 | 53.461 | 23.421 | 106.888 | 1.00 | 19.54 | A | C |
| ATOM | 976 | CD2 | LEU | A | 407 | 54.629 | 21.766 | 105.447 | 1.00 | 25.90 | A | C |
| ATOM | 977 | C | LEU | A | 407 | 56.921 | 25.103 | 103.694 | 1.00 | 23.47 | A | C |
| ATOM | 978 | O | LEU | A | 407 | 56.093 | 25.422 | 102.844 | 1.00 | 23.57 | A | O |
| ATOM | 979 | N | GLY | A | 408 | 58.077 | 25.743 | 103.852 | 1.00 | 22.90 | A | N |
| ATOM | 980 | CA | GLY | A | 408 | 58.444 | 26.847 | 102.983 | 1.00 | 23.36 | A | C |
| ATOM | 981 | C | GLY | A | 408 | 58.411 | 28.268 | 103.528 | 1.00 | 24.25 | A | C |
| ATOM | 982 | O | GLY | A | 408 | 58.891 | 29.190 | 102.868 | 1.00 | 22.84 | A | O |
| ATOM | 983 | N | LEU | A | 409 | 57.855 | 28.463 | 104.719 | 1.00 | 25.11 | A | N |
| ATOM | 984 | CA | LEU | A | 409 | 57.777 | 29.804 | 105.290 | 1.00 | 24.70 | A | C |
| ATOM | 985 | CB | LEU | A | 409 | 56.853 | 29.828 | 106.508 | 1.00 | 22.64 | A | C |
| ATOM | 986 | CG | LEU | A | 409 | 55.352 | 29.682 | 106.281 | 1.00 | 25.21 | A | C |
| ATOM | 987 | CD1 | LEU | A | 409 | 54.639 | 29.943 | 107.588 | 1.00 | 21.69 | A | C |
| ATOM | 988 | CD2 | LEU | A | 409 | 54.880 | 30.664 | 105.212 | 1.00 | 24.00 | A | C |
| ATOM | 989 | C | LEU | A | 409 | 59.123 | 30.347 | 105.711 | 1.00 | 25.87 | A | C |
| ATOM | 990 | O | LEU | A | 409 | 59.989 | 29.598 | 106.156 | 1.00 | 26.76 | A | O |
| ATOM | 991 | N | ASP | A | 410 | 59.294 | 31.656 | 105.558 | 1.00 | 27.29 | A | N |
| ATOM | 992 | CA | ASP | A | 410 | 60.517 | 32.323 | 105.980 | 1.00 | 29.12 | A | C |
| ATOM | 993 | CB | ASP | A | 410 | 60.990 | 33.338 | 104.933 | 1.00 | 34.79 | A | C |
| ATOM | 994 | CG | ASP | A | 410 | 61.553 | 32.678 | 103.687 | 1.00 | 42.97 | A | C |
| ATOM | 995 | OD1 | ASP | A | 410 | 62.184 | 31.602 | 103.799 | 1.00 | 48.11 | A | O |
| ATOM | 996 | OD2 | ASP | A | 410 | 61.381 | 33.247 | 102.590 | 1.00 | 47.38 | A | O |
| ATOM | 997 | C | ASP | A | 410 | 60.188 | 33.050 | 107.289 | 1.00 | 27.33 | A | C |
| ATOM | 998 | O | ASP | A | 410 | 59.046 | 33.026 | 107.751 | 1.00 | 19.85 | A | O |
| ATOM | 999 | N | HIS | A | 411 | 61.190 | 33.687 | 107.883 | 1.00 | 25.92 | A | N |
| ATOM | 1000 | CA | HIS | A | 411 | 60.995 | 34.418 | 109.128 | 1.00 | 27.93 | A | C |
| ATOM | 1001 | CB | HIS | A | 411 | 62.345 | 34.843 | 109.699 | 1.00 | 25.18 | A | C |
| ATOM | 1002 | CG | HIS | A | 411 | 63.056 | 33.747 | 110.430 | 1.00 | 25.67 | A | C |
| ATOM | 1003 | CD2 | HIS | A | 411 | 62.579 | 32.692 | 111.134 | 1.00 | 22.64 | A | C |
| ATOM | 1004 | ND1 | HIS | A | 411 | 64.429 | 33.660 | 110.493 | 1.00 | 24.52 | A | N |
| ATOM | 1005 | CE1 | HIS | A | 411 | 64.768 | 32.596 | 111.199 | 1.00 | 24.55 | A | C |
| ATOM | 1006 | NE2 | HIS | A | 411 | 63.664 | 31.992 | 111.598 | 1.00 | 21.02 | A | N |
| ATOM | 1007 | C | HIS | A | 411 | 60.103 | 35.634 | 108.947 | 1.00 | 28.39 | A | C |
| ATOM | 1008 | O | HIS | A | 411 | 60.096 | 36.257 | 107.890 | 1.00 | 27.54 | A | O |
| ATOM | 1009 | N | SER | A | 412 | 59.348 | 35.953 | 109.992 | 1.00 | 28.70 | A | N |
| ATOM | 1010 | CA | SER | A | 412 | 58.441 | 37.090 | 109.989 | 1.00 | 28.94 | A | C |
| ATOM | 1011 | CB | SER | A | 412 | 57.139 | 36.716 | 110.696 | 1.00 | 27.79 | A | C |
| ATOM | 1012 | CG | SER | A | 412 | 56.288 | 37.836 | 110.831 | 1.00 | 28.82 | A | O |
| ATOM | 1013 | C | SER | A | 412 | 59.090 | 38.264 | 110.708 | 1.00 | 30.69 | A | C |
| ATOM | 1014 | O | SER | A | 412 | 60.020 | 38.081 | 111.489 | 1.00 | 31.96 | A | O |
| ATOM | 1015 | N | SER | A | 413 | 58.601 | 39.468 | 110.437 | 1.00 | 30.98 | A | N |
| ATOM | 1016 | CA | SER | A | 413 | 59.132 | 40.664 | 111.075 | 1.00 | 33.39 | A | C |
| ATOM | 1017 | CB | SER | A | 413 | 59.334 | 41.775 | 110.039 | 1.00 | 34.31 | A | C |
| ATOM | 1018 | CG | SER | A | 413 | 58.154 | 42.003 | 109.294 | 1.00 | 35.39 | A | O |
| ATOM | 1019 | C | SER | A | 413 | 58.175 | 41.125 | 112.166 | 1.00 | 32.91 | A | C |
| ATOM | 1020 | O | SER | A | 413 | 58.454 | 42.076 | 112.893 | 1.00 | 35.58 | A | O |
| ATOM | 1021 | N | VAL | A | 414 | 57.043 | 40.436 | 112.264 | 1.00 | 31.58 | A | N |
| ATOM | 1022 | CA | VAL | A | 414 | 56.023 | 40.725 | 113.264 | 1.00 | 31.56 | A | C |
| ATOM | 1023 | GB | VAL | A | 414 | 54.637 | 40.234 | 112.792 | 1.00 | 31.76 | A | C |
| ATOM | 1024 | CG1 | VAL | A | 414 | 53.578 | 40.582 | 113.816 | 1.00 | 31.52 | A | C |
| ATOM | 1025 | CG2 | VAL | A | 414 | 54.304 | 40.849 | 111.455 | 1.00 | 30.36 | A | C |
| ATOM | 1026 | C | VAL | A | 414 | 56.399 | 40.007 | 114.564 | 1.00 | 31.65 | A | C |
| ATOM | 1027 | O | VAL | A | 414 | 56.415 | 38.778 | 114.630 | 1.00 | 30.34 | A | O |
| ATOM | 1028 | N | PRO | A | 415 | 56.696 | 40.775 | 115.620 | 1.00 | 31.44 | A | N |
| ATOM | 1029 | CD | PRO | A | 415 | 56.730 | 42.248 | 115.656 | 1.00 | 29.01 | A | C |
| ATOM | 1030 | CA | PRO | A | 415 | 57.080 | 40.220 | 116.920 | 1.00 | 28.81 | A | C |
| ATOM | 1031 | CB | PRO | A | 415 | 57.116 | 41.461 | 117.802 | 1.00 | 29.03 | A | C |
| ATOM | 1032 | CG | PRO | A | 415 | 57.628 | 42.513 | 116.837 | 1.00 | 27.34 | A | C |
| ATOM | 1033 | C | PRO | A | 415 | 56.156 | 39.157 | 117.503 | 1.00 | 26.99 | A | C |
| ATOM | 1034 | O | PRO | A | 415 | 56.626 | 38.199 | 118.098 | 1.00 | 26.82 | A | O |
| ATOM | 1035 | N | GLU | A | 416 | 54.849 | 39.316 | 117.336 | 1.00 | 28.55 | A | N |
| ATOM | 1036 | CA | GLU | A | 416 | 53.907 | 38.343 | 117.881 | 1.00 | 29.16 | A | C |
| ATOM | 1037 | CB | GLU | A | 416 | 52.597 | 39.029 | 118.276 | 1.00 | 33.01 | A | C |
| ATOM | 1038 | CG | GLU | A | 416 | 52.180 | 40.147 | 117.341 | 1.00 | 42.66 | A | C |
| ATOM | 1039 | CD | GLU | A | 416 | 52.947 | 41.437 | 117.590 | 1.00 | 43.72 | A | C |
| ATOM | 1040 | OE1 | GLU | A | 416 | 53.148 | 42.204 | 116.626 | 1.00 | 43.99 | A | O |
| ATOM | 1041 | OE2 | GLU | A | 416 | 53.336 | 41.690 | 118.752 | 1.00 | 46.02 | A | O |
| ATOM | 1042 | C | GLU | A | 416 | 53.612 | 37.163 | 116.957 | 1.00 | 28.85 | A | C |
| ATOM | 1043 | O | GLU | A | 416 | 52.820 | 36.289 | 117.297 | 1.00 | 30.41 | A | O |
| ATOM | 1044 | N | ALA | A | 417 | 54.257 | 37.125 | 115.798 | 1.00 | 26.45 | A | N |
| ATOM | 1045 | CA | ALA | A | 417 | 54.037 | 36.029 | 114.867 | 1.00 | 24.95 | A | C |
| ATOM | 1046 | CB | ALA | A | 417 | 54.429 | 36.452 | 113.456 | 1.00 | 23.05 | A | C |
| ATOM | 1047 | C | ALA | A | 417 | 54.868 | 34.826 | 115.303 | 1.00 | 24.52 | A | C |
| ATOM | 1048 | O | ALA | A | 417 | 55.952 | 34.986 | 115.861 | 1.00 | 23.59 | A | O |
| ATOM | 1049 | N | LEU | A | 418 | 54.354 | 33.625 | 115.049 | 1.00 | 22.60 | A | N |
| ATOM | 1050 | CA | LEU | A | 418 | 55.060 | 32.399 | 115.396 | 1.00 | 21.25 | A | C |
| ATOM | 1051 | CB | LEU | A | 418 | 54.186 | 31.183 | 115.084 | 1.00 | 24.09 | A | C |
| ATOM | 1052 | CG | LEU | A | 418 | 54.819 | 29.794 | 115.255 | 1.00 | 25.61 | A | C |
| ATOM | 1053 | CD1 | LEU | A | 418 | 55.205 | 29.584 | 116.704 | 1.00 | 24.01 | A | C |
| ATOM | 1054 | CD2 | LEU | A | 418 | 53.851 | 28.711 | 114.802 | 1.00 | 24.22 | A | C |
| ATOM | 1055 | C | LEU | A | 418 | 56.378 | 32.281 | 114.639 | 1.00 | 20.33 | A | C |
| ATOM | 1056 | O | LEU | A | 418 | 57.336 | 31.699 | 115.139 | 1.00 | 17.86 | A | O |
| ATOM | 1057 | N | MET | A | 419 | 56.425 | 32.836 | 113.431 | 1.00 | 20.15 | A | N |
| ATOM | 1058 | CA | MET | A | 419 | 57.632 | 32.763 | 112.620 | 1.00 | 20.11 | A | C |
| ATOM | 1059 | CB | MET | A | 419 | 57.286 | 32.817 | 111.130 | 1.00 | 19.41 | A | C |
| ATOM | 1060 | CG | MET | A | 419 | 56.562 | 31.575 | 110.616 | 1.00 | 18.20 | A | C |
| ATOM | 1061 | SD | MET | A | 419 | 57.267 | 29.973 | 111.160 | 1.00 | 18.41 | A | S |
| ATOM | 1062 | CE | MET | A | 419 | 58.852 | 29.969 | 110.370 | 1.00 | 13.77 | A | C |
| ATOM | 1063 | C | MET | A | 419 | 58.678 | 33.816 | 112.953 | 1.00 | 20.18 | A | C |
| ATOM | 1064 | O | MET | A | 419 | 59.679 | 33.946 | 112.256 | 1.00 | 20.02 | A | O |
| ATOM | 1065 | N | TYR | A | 420 | 58.450 | 34.571 | 114.022 | 1.00 | 22.13 | A | N |
| ATOM | 1066 | CA | TYR | A | 420 | 59.433 | 35.561 | 114.443 | 1.00 | 23.96 | A | C |
| ATOM | 1067 | CB | TYR | A | 420 | 58.921 | 36.336 | 115.653 | 1.00 | 28.74 | A | C |
| ATOM | 1068 | CG | TYR | A | 420 | 59.721 | 37.578 | 115.957 | 1.00 | 33.66 | A | C |
| ATOM | 1069 | CD1 | TYR | A | 420 | 59.747 | 38.654 | 115.064 | 1.00 | 35.38 | A | C |
| ATOM | 1070 | GE1 | TYR | A | 420 | 60.495 | 39.791 | 115.334 | 1.00 | 36.85 | A | C |
| ATOM | 1071 | CD2 | TYR | A | 420 | 60.464 | 37.674 | 117.128 | 1.00 | 35.51 | A | C |
| ATOM | 1072 | CE2 | TYR | A | 420 | 61.215 | 38.804 | 117.409 | 1.00 | 38.35 | A | C |
| ATOM | 1073 | CZ | TYR | A | 420 | 61.228 | 39.858 | 116.512 | 1.00 | 40.13 | A | C |
| ATOM | 1074 | OH | TYR | A | 420 | 61.973 | 40.976 | 116.801 | 1.00 | 41.02 | A | O |
| ATOM | 1075 | C | TYR | A | 420 | 60.679 | 34.746 | 114.812 | 1.00 | 22.16 | A | C |
| ATOM | 1076 | O | TYR | A | 420 | 60.583 | 33.744 | 115.515 | 1.00 | 20.07 | A | O |
| ATOM | 1077 | N | PRO | A | 421 | 61.863 | 35.172 | 114.347 | 1.00 | 23.33 | A | N |
| ATOM | 1078 | CD | PRO | A | 421 | 62.072 | 36.447 | 113.634 | 1.00 | 24.71 | A | C |
| ATOM | 1079 | CA | PRO | A | 421 | 63.148 | 34.501 | 114.591 | 1.00 | 24.11 | A | C |
| ATOM | 1080 | CB | PRO | A | 421 | 64.129 | 35.342 | 113.773 | 1.00 | 24.83 | A | C |
| ATOM | 1081 | CG | PRO | A | 421 | 63.542 | 36.703 | 113.850 | 1.00 | 23.46 | A | C |
| ATOM | 1082 | C | PRO | A | 421 | 63.624 | 34.301 | 116.028 | 1.00 | 24.67 | A | C |
| ATOM | 1083 | O | PRO | A | 421 | 64.649 | 33.659 | 116.258 | 1.00 | 23.19 | A | O |
| ATOM | 1084 | N | MET | A | 422 | 62.870 | 34.827 | 116.986 | 1.00 | 23.77 | A | N |
| ATOM | 1085 | CA | MET | A | 422 | 63.228 | 34.732 | 118.393 | 1.00 | 24.04 | A | C |
| ATOM | 1086 | CB | MET | A | 422 | 63.351 | 36.148 | 118.961 | 1.00 | 29.95 | A | C |
| ATOM | 1087 | CG | MET | A | 422 | 63.671 | 36.231 | 120.433 | 1.00 | 39.49 | A | C |
| ATOM | 1088 | SD | MET | A | 422 | 63.479 | 37.927 | 121.034 | 1.00 | 49.06 | A | S |
| ATOM | 1089 | CE | MET | A | 422 | 61.893 | 37.842 | 121.866 | 1.00 | 39.08 | A | C |
| ATOM | 1090 | C | MET | A | 422 | 62.181 | 33.938 | 119.171 | 1.00 | 22.85 | A | C |
| ATOM | 1091 | O | MET | A | 422 | 60.983 | 34.062 | 118.915 | 1.00 | 21.77 | A | O |
| ATOM | 1092 | N | TYR | A | 423 | 62.626 | 33.117 | 120.114 | 1.00 | 19.77 | A | N |
| ATOM | 1093 | CA | TYR | A | 423 | 61.699 | 32.324 | 120.913 | 1.00 | 21.78 | A | C |
| ATOM | 1094 | CB | TYR | A | 423 | 62.441 | 31.214 | 121.659 | 1.00 | 19.09 | A | C |
| ATOM | 1095 | CG | TYR | A | 423 | 61.551 | 30.425 | 122.599 | 1.00 | 18.13 | A | C |
| ATOM | 1096 | GD1 | TYR | A | 423 | 60.797 | 29.344 | 122.139 | 1.00 | 18.66 | A | C |
| ATOM | 1097 | CE1 | TYR | A | 423 | 59.962 | 28.623 | 122.996 | 1.00 | 16.35 | A | C |
| ATOM | 1098 | CD2 | TYR | A | 423 | 61.445 | 30.769 | 123.946 | 1.00 | 17.67 | A | C |
| ATOM | 1099 | CE2 | TYR | A | 423 | 60.612 | 30.059 | 124.811 | 1.00 | 17.18 | A | C |
| ATOM | 1100 | CZ | TYR | A | 423 | 59.874 | 28.987 | 124.330 | 1.00 | 19.92 | A | C |
| ATOM | 1101 | OH | TYR | A | 423 | 59.053 | 28.278 | 125.182 | 1.00 | 18.58 | A | O |
| ATOM | 1102 | C | TYR | A | 423 | 60.964 | 33.168 | 121.950 | 1.00 | 23.28 | A | C |
| ATOM | 1103 | O | TYR | A | 423 | 61.568 | 34.001 | 122.616 | 1.00 | 23.95 | A | O |
| ATOM | 1104 | N | ARG | A | 424 | 59.662 | 32.942 | 122.087 | 1.00 | 25.46 | A | N |
| ATOM | 1105 | CA | ARG | A | 424 | 58.869 | 33.651 | 123.084 | 1.00 | 31.01 | A | C |
| ATOM | 1106 | CB | ARG | A | 424 | 58.376 | 35.005 | 122.550 | 1.00 | 35.79 | A | C |
| ATOM | 1107 | CO | ARG | A | 424 | 57.230 | 34.932 | 121.573 | 1.00 | 43.60 | A | C |
| ATOM | 1108 | CD | ARG | A | 424 | 56.693 | 36.313 | 121.235 | 1.00 | 50.63 | A | C |
| ATOM | 1109 | NE | ARG | A | 424 | 55.481 | 36.228 | 120.420 | 1.00 | 58.11 | A | N |
| ATOM | 1110 | CZ | ARG | A | 424 | 54.340 | 35.676 | 120.828 | 1.00 | 62.35 | A | C |
| ATOM | 1111 | NH1 | ARG | A | 424 | 54.247 | 35.158 | 122.049 | 1.00 | 65.08 | A | N |
| ATOM | 1112 | NH2 | ARG | A | 424 | 53.291 | 35.634 | 120.015 | 1.00 | 62.97 | A | N |
| ATOM | 1113 | C | ARG | A | 424 | 57.690 | 32.777 | 123.499 | 1.00 | 29.80 | A | C |
| ATOM | 1114 | O | ARG | A | 424 | 56.830 | 32.450 | 122.691 | 1.00 | 29.90 | A | O |
| ATOM | 1115 | N | PHE | A | 425 | 57.654 | 32.374 | 124.762 | 1.00 | 29.63 | A | N |
| ATOM | 1116 | CA | PHE | A | 425 | 56.555 | 31.534 | 125.203 | 1.00 | 28.26 | A | C |
| ATOM | 1117 | CB | PHE | A | 425 | 56.807 | 30.963 | 126.596 | 1.00 | 24.65 | A | C |
| ATOM | 1118 | CG | PHE | A | 425 | 55.660 | 30.152 | 127.112 | 1.00 | 23.69 | A | G |
| ATOM | 1119 | CD1 | PHE | A | 425 | 54.673 | 30.740 | 127.898 | 1.00 | 22.35 | A | C |
| ATOM | 1120 | CD2 | PHE | A | 425 | 55.506 | 28.820 | 126.729 | 1.00 | 21.86 | A | C |
| ATOM | 1121 | CE1 | PHE | A | 425 | 53.542 | 30.016 | 128.288 | 1.00 | 23.01 | A | C |
| ATOM | 1122 | CE2 | PHE | A | 425 | 54.379 | 28.086 | 127.113 | 1.00 | 21.25 | A | C |
| ATOM | 1123 | CZ | PHE | A | 425 | 53.395 | 28.684 | 127.892 | 1.00 | 21.91 | A | C |
| ATOM | 1124 | C | PHE | A | 425 | 55.236 | 32.287 | 125.205 | 1.00 | 29.92 | A | C |
| ATOM | 1125 | O | PHE | A | 425 | 55.177 | 33.475 | 125.525 | 1.00 | 32.27 | A | O |
| ATOM | 1126 | N | THR | A | 426 | 54.172 | 31.586 | 124.843 | 1.00 | 28.15 | A | N |
| ATOM | 1127 | CA | THR | A | 426 | 52.858 | 32.195 | 124.817 | 1.00 | 28.89 | A | C |
| ATOM | 1128 | CB | THR | A | 426 | 52.581 | 32.896 | 123.463 | 1.00 | 30.65 | A | C |
| ATOM | 1129 | OG1 | THR | A | 426 | 51.258 | 33.445 | 123.476 | 1.00 | 30.50 | A | O |
| ATOM | 1130 | CG2 | THR | A | 426 | 52.692 | 31.913 | 122.309 | 1.00 | 27.51 | A | C |
| ATOM | 1131 | C | THR | A | 426 | 51.771 | 31.168 | 125.054 | 1.00 | 28.39 | A | C |
| ATOM | 1132 | O | THR | A | 426 | 51.936 | 29.991 | 124.763 | 1.00 | 27.43 | A | O |
| ATOM | 1133 | N | GLU | A | 427 | 50.662 | 31.627 | 125.615 | 1.00 | 31.92 | A | N |
| ATOM | 1134 | CA | GLU | A | 427 | 49.515 | 30.767 | 125.864 | 1.00 | 33.81 | A | C |
| ATOM | 1135 | CB | GLU | A | 427 | 48.931 | 31.032 | 127.252 | 1.00 | 35.60 | A | C |
| ATOM | 1136 | CG | GLU | A | 427 | 49.820 | 30.574 | 128.402 | 1.00 | 41.56 | A | C |
| ATOM | 1137 | CD | GLU | A | 427 | 49.178 | 30.786 | 129.765 | 1.00 | 43.70 | A | C |
| ATOM | 1138 | OE1 | GLU | A | 427 | 48.985 | 31.959 | 130.162 | 1.00 | 45.79 | A | O |
| ATOM | 1139 | OE2 | GLU | A | 427 | 48.865 | 29.779 | 130.437 | 1.00 | 43.08 | A | O |
| ATOM | 1140 | C | GLU | A | 427 | 48.501 | 31.133 | 124.785 | 1.00 | 33.55 | A | C |
| ATOM | 1141 | O | GLU | A | 427 | 48.634 | 32.165 | 124.127 | 1.00 | 32.48 | A | O |
| ATOM | 1142 | N | GLY | A | 428 | 47.492 | 30.297 | 124.596 | 1.00 | 32.38 | A | N |
| ATOM | 1143 | CA | GLY | A | 428 | 46.509 | 30.596 | 123.575 | 1.00 | 34.96 | A | C |
| ATOM | 1144 | C | GLY | A | 428 | 47.015 | 30.234 | 122.189 | 1.00 | 35.38 | A | C |
| ATOM | 1145 | O | GLY | A | 428 | 48.137 | 29.740 | 122.050 | 1.00 | 34.55 | A | O |
| ATOM | 1146 | N | PRO | A | 429 | 46.210 | 30.482 | 121.139 | 1.00 | 35.27 | A | N |
| ATOM | 1147 | CD | PRO | A | 429 | 44.979 | 31.286 | 121.203 | 1.00 | 35.48 | A | C |
| ATOM | 1148 | CA | PRO | A | 429 | 46.555 | 30.185 | 119.744 | 1.00 | 32.90 | A | C |
| ATOM | 1149 | CB | PRO | A | 429 | 45.396 | 30.803 | 118.966 | 1.00 | 33.89 | A | C |
| ATOM | 1150 | CO | PRO | A | 429 | 44.964 | 31.931 | 119.848 | 1.00 | 34.64 | A | C |
| ATOM | 1151 | C | PRO | A | 429 | 47.913 | 30.740 | 119.329 | 1.00 | 30.15 | A | C |
| ATOM | 1152 | O | PRO | A | 429 | 48.156 | 31.939 | 119.403 | 1.00 | 32.20 | A | O |
| ATOM | 1153 | N | PRO | A | 430 | 48.817 | 29.858 | 118.886 | 1.00 | 27.85 | A | N |
| ATOM | 1154 | CD | PRO | A | 430 | 48.637 | 28.397 | 118.903 | 1.00 | 26.06 | A | C |
| ATOM | 1155 | CA | PRO | A | 430 | 50.173 | 30.211 | 118.450 | 1.00 | 27.78 | A | C |
| ATOM | 1156 | CB | PRO | A | 430 | 50.869 | 28.849 | 118.335 | 1.00 | 28.30 | A | C |
| ATOM | 1157 | CG | PRO | A | 430 | 50.029 | 27.927 | 119.193 | 1.00 | 29.20 | A | C |
| ATOM | 1158 | C | PRO | A | 430 | 50.260 | 30.989 | 117.135 | 1.00 | 27.63 | A | C |
| ATOM | 1159 | O | PRO | A | 43O | 51.068 | 31.909 | 117.000 | 1.00 | 26.32 | A | O |
| ATOM | 1160 | N | LEU | A | 431 | 49.437 | 30.611 | 116.164 | 1.00 | 26.85 | A | N |
| ATOM | 1161 | CA | LEU | A | 431 | 49.470 | 31.257 | 114.858 | 1.00 | 28.30 | A | C |
| ATOM | 1162 | CB | LEU | A | 431 | 48.752 | 30.395 | 113.815 | 1.00 | 26.29 | A | C |
| ATOM | 1163 | CG | LEU | A | 431 | 49.353 | 29.024 | 113.507 | 1.00 | 26.78 | A | C |
| ATOM | 1164 | CD1 | LEU | A | 431 | 48.615 | 28.430 | 112.326 | 1.00 | 27.79 | A | C |
| ATOM | 1165 | CD2 | LEU | A | 431 | 50.827 | 29.144 | 113.188 | 1.00 | 25.85 | A | C |
| ATOM | 1166 | C | LEU | A | 431 | 48.897 | 32.658 | 114.815 | 1.00 | 26.95 | A | C |
| ATOM | 1167 | O | LEU | A | 431 | 47.776 | 32.891 | 115.248 | 1.00 | 27.17 | A | O |
| ATOM | 1168 | N | HIS | A | 432 | 49.681 | 33.585 | 114.279 | 1.00 | 27.31 | A | N |
| ATOM | 1169 | CA | HIS | A | 432 | 49.262 | 34.971 | 114.144 | 1.00 | 29.66 | A | C |
| ATOM | 1170 | CB | HIS | A | 432 | 50.469 | 35.883 | 114.367 | 1.00 | 30.80 | A | C |
| ATOM | 1171 | CO | HIS | A | 432 | 50.157 | 37.342 | 114.263 | 1.00 | 35.25 | A | C |
| ATOM | 1172 | CD2 | HIS | A | 432 | 50.006 | 38.291 | 115.216 | 1.00 | 36.05 | A | C |
| ATOM | 1173 | ND1 | HIS | A | 432 | 49.973 | 37.980 | 113.056 | 1.00 | 35.61 | A | N |
| ATOM | 1174 | CE1 | HIS | A | 432 | 49.724 | 39.260 | 113.269 | 1.00 | 35.01 | A | C |
| ATOM | 1175 | NE2 | HIS | A | 432 | 49.739 | 39.474 | 114.571 | 1.00 | 37.63 | A | N |
| ATOM | 1176 | C | H1S | A | 432 | 48.688 | 35.148 | 112.729 | 1.00 | 31.08 | A | C |
| ATOM | 1177 | O | HIS | A | 432 | 48.989 | 34.353 | 111.843 | 1.00 | 31.29 | A | O |
| ATOM | 1178 | N | LYS | A | 433 | 47.855 | 36.169 | 112.525 | 1.00 | 30.77 | A | N |
| ATOM | 1179 | CA | LYS | A | 433 | 47.252 | 36.437 | 111.214 | 1.00 | 31.04 | A | C |
| ATOM | 1180 | GB | LYS | A | 433 | 46.590 | 37.817 | 111.186 | 1.00 | 36.69 | A | C |
| ATOM | 1181 | CG | LYS | A | 433 | 45.486 | 38.010 | 112.205 | 1.00 | 46.32 | A | C |
| ATOM | 1182 | CD | LYS | A | 433 | 45.180 | 39.489 | 112.428 | 1.00 | 52.39 | A | C |
| ATOM | 1183 | CE | LYS | A | 433 | 44.194 | 39.680 | 113.585 | 1.00 | 57.26 | A | C |
| ATOM | 1184 | NZ | LYS | A | 433 | 43.913 | 41.116 | 113.889 | 1.00 | 59.05 | A | N |
| ATOM | 1185 | C | LYS | A | 433 | 48.328 | 36.411 | 110.148 | 1.00 | 30.86 | A | C |
| ATOM | 1186 | O | LYS | A | 433 | 48.111 | 35.954 | 109.025 | 1.00 | 28.27 | A | O |
| ATOM | 1187 | N | ASP | A | 434 | 49.496 | 36.919 | 110.511 | 1.00 | 29.06 | A | N |
| ATOM | 1188 | CA | ASP | A | 434 | 50.609 | 36.960 | 109.591 | 1.00 | 26.69 | A | C |
| ATOM | 1189 | CB | ASP | A | 434 | 51.766 | 37.732 | 110.213 | 1.00 | 26.22 | A | C |
| ATOM | 1190 | CG | ASP | A | 434 | 52.923 | 37.905 | 109.258 | 1.00 | 27.32 | A | C |
| ATOM | 1191 | OD1 | ASP | A | 434 | 53.916 | 37.168 | 109.388 | 1.00 | 27.96 | A | O |
| ATOM | 1192 | OD2 | ASP | A | 434 | 52.837 | 38.774 | 108.366 | 1.00 | 29.60 | A | O |
| ATOM | 1193 | C | ASP | A | 434 | 51.052 | 35.557 | 109.202 | 1.00 | 27.04 | A | C |
| ATOM | 1194 | O | ASP | A | 434 | 51.350 | 35.300 | 108.039 | 1.00 | 26.95 | A | O |
| ATOM | 1195 | N | ASP | A | 435 | 51.086 | 34.644 | 110.169 | 1.00 | 26.04 | A | N |
| ATOM | 1196 | CA | ASP | A | 435 | 51.496 | 33.270 | 109.890 | 1.00 | 26.18 | A | C |
| ATOM | 1197 | CB | ASP | A | 435 | 51.629 | 32.453 | 111.186 | 1.00 | 25.76 | A | C |
| ATOM | 1198 | CG | ASP | A | 435 | 52.659 | 33.026 | 112.159 | 1.00 | 23.37 | A | C |
| ATOM | 1199 | OD1 | ASP | A | 43S | 53.833 | 33.231 | 111.778 | 1.00 | 17.56 | A | O |
| ATOM | 1200 | OD2 | ASP | A | 435 | 52.281 | 33.257 | 113.319 | 1.00 | 21.37 | A | O |
| ATOM | 1201 | C | ASP | A | 435 | 50.483 | 32.586 | 108.979 | 1.00 | 26.53 | A | C |
| ATOM | 1202 | O | ASP | A | 435 | 50.857 | 31.854 | 108.065 | 1.00 | 27.59 | A | O |
| ATOM | 1203 | N | VAL | A | 436 | 49.202 | 32.824 | 109.243 | 1.00 | 26.10 | A | N |
| ATOM | 1204 | CA | VAL | A | 436 | 48.123 | 32.238 | 108.461 | 1.00 | 27.17 | A | C |
| ATOM | 1205 | CB | VAL | A | 436 | 46.758 | 32.516 | 109.116 | 1.00 | 28.67 | A | C |
| ATOM | 1206 | CG1 | VAL | A | 436 | 45.651 | 31.841 | 108.322 | 1.00 | 28.01 | A | C |
| ATOM | 1207 | CG2 | VAL | A | 436 | 46.760 | 32.012 | 110.546 | 1.00 | 27.09 | A | C |
| ATOM | 1208 | C | VAL | A | 436 | 48.105 | 32.755 | 107.021 | 1.00 | 28.36 | A | C |
| ATOM | 1209 | O | VAL | A | 436 | 47.814 | 32.006 | 166.096 | 1.00 | 28.48 | A | O |
| ATOM | 1210 | N | ASN | A | 437 | 48.411 | 34.034 | 106.832 | 1.00 | 30.05 | A | N |
| ATOM | 1211 | CA | ASN | A | 437 | 48.449 | 34.604 | 105.488 | 1.00 | 32.08 | A | C |
| ATOM | 1212 | CB | ASN | A | 437 | 48.670 | 36.121 | 105.525 | 1.00 | 36.19 | A | C |
| ATOM | 1213 | CG | ASN | A | 437 | 47.464 | 36.875 | 106.018 | 1.00 | 40.19 | A | C |
| ATOM | 1214 | OD1 | ASN | A | 437 | 46.328 | 36.484 | 105.759 | 1.00 | 44.41 | A | O |
| ATOM | 1215 | ND2 | ASN | A | 437 | 47.701 | 37.978 | 106.719 | 1.00 | 44.69 | A | N |
| ATOM | 1216 | C | ASN | A | 437 | 49.589 | 33.983 | 104.701 | 1.00 | 30.92 | A | C |
| ATOM | 1217 | O | ASN | A | 437 | 49.406 | 33.559 | 103.565 | 1.00 | 31.67 | A | O |
| ATOM | 1218 | N | GLY | A | 438 | 50.768 | 33.946 | 105.315 | 1.00 | 27.54 | A | N |
| ATOM | 1219 | CA | GLY | A | 438 | 51.931 | 33.385 | 104.659 | 1.00 | 25.40 | A | C |
| ATOM | 1220 | C | GLY | A | 438 | 51.755 | 31.952 | 104.205 | 1.00 | 24.52 | A | C |
| ATOM | 1221 | O | GLY | A | 438 | 52.171 | 31.593 | 103.110 | 1.00 | 26.30 | A | O |
| ATOM | 1222 | N | ILE | A | 439 | 51.137 | 31.125 | 105.039 | 1.00 | 23.54 | A | N |
| ATOM | 1223 | CA | ILE | A | 439 | 50.943 | 29.732 | 104.681 | 1.00 | 23.31 | A | C |
| ATOM | 1224 | CB | ILE | A | 439 | 50.619 | 28.871 | 105.944 | 1.00 | 20.22 | A | C |
| ATOM | 1225 | CG2 | ILE | A | 439 | 49.210 | 29.136 | 106.450 | 1.00 | 18.09 | A | C |
| ATOM | 1226 | CG1 | ILE | A | 439 | 50.814 | 27.393 | 105.614 | 1.00 | 21.12 | A | C |
| ATOM | 1227 | CD1 | ILE | A | 439 | 52.238 | 27.048 | 105.201 | 1.00 | 14.56 | A | C |
| ATOM | 1228 | C | ILE | A | 439 | 49.867 | 29.547 | 103.593 | 1.00 | 24.64 | A | C |
| ATOM | 1229 | O | ILE | A | 439 | 50.017 | 28.712 | 102.705 | 1.00 | 25.35 | A | O |
| ATOM | 1230 | N | ARG | A | 440 | 48.793 | 30.324 | 103.649 | 1.00 | 23.43 | A | N |
| ATOM | 1231 | CA | ARG | A | 440 | 47.751 | 30.217 | 102.638 | 1.00 | 24.74 | A | C |
| ATOM | 1232 | CB | ARG | A | 440 | 46.481 | 30.932 | 103.091 | 1.00 | 25.89 | A | C |
| ATOM | 1233 | CG | ARG | A | 440 | 45.673 | 30.154 | 104.093 | 1.00 | 28.05 | A | C |
| ATOM | 1234 | CD | ARG | A | 440 | 44.453 | 30.927 | 104.518 | 1.00 | 33.06 | A | C |
| ATOM | 1235 | NE | ARG | A | 440 | 43.678 | 30.183 | 105.502 | 1.00 | 40.49 | A | N |
| ATOM | 1236 | CZ | ARG | A | 440 | 42.752 | 30.722 | 106.286 | 1.00 | 43.77 | A | C |
| ATOM | 1237 | NH1 | ARG | A | 440 | 42.485 | 32.020 | 106.205 | 1.00 | 45.47 | A | N |
| ATOM | 1238 | NH2 | ARG | A | 440 | 42.089 | 29.964 | 107.150 | 1.00 | 43.22 | A | N |
| ATOM | 1239 | C | ARG | A | 440 | 48.217 | 30.806 | 101.312 | 1.00 | 26.05 | A | C |
| ATOM | 1240 | O | ARO | A | 440 | 47.691 | 30.466 | 100.257 | 1.00 | 29.07 | A | O |
| ATOM | 1241 | N | HIS | A | 441 | 49.201 | 31.694 | 101.360 | 1.00 | 24.35 | A | N |
| ATOM | 1242 | CA | HIS | A | 441 | 49.707 | 32.290 | 100.139 | 1.00 | 24.72 | A | C |
| ATOM | 1243 | CB | HIS | A | 441 | 50.628 | 33.465 | 100.450 | 1.00 | 21.94 | A | C |
| ATOM | 1244 | CG | HiS | A | 441 | 50.942 | 34.308 | 99.255 | 1.00 | 21.49 | A | C |
| ATOM | 1245 | CD2 | HIS | A | 441 | 52.068 | 34.421 | 98.513 | 1.00 | 23.26 | A | C |
| ATOM | 1246 | ND1 | HIS | A | 441 | 50.018 | 35.152 | 98.679 | 1.00 | 23.21 | A | N |
| ATOM | 1247 | CE1 | HIS | A | 441 | 50.561 | 35.748 | 97.634 | 1.00 | 21.93 | A | C |
| ATOM | 1248 | NE2 | HIS | A | 441 | 51.804 | 35.322 | 97.512 | 1.00 | 24.09 | A | N |
| ATOM | 1249 | C | HIS | A | 441 | 50.463 | 31.238 | 99.332 | 1.00 | 26.24 | A | C |
| ATOM | 1250 | O | HIS | A | 441 | 50.531 | 31.313 | 98.104 | 1.00 | 26.96 | A | O |
| ATOM | 1251 | N | LEU | A | 442 | 51.028 | 30.260 | 100.032 | 1.00 | 27.09 | A | N |
| ATOM | 1252 | CA | LEU | A | 442 | 51.770 | 29.178 | 99.394 | 1.00 | 27.80 | A | C |
| ATOM | 1253 | CB | LEU | A | 442 | 52.861 | 28.648 | 100.329 | 1.00 | 28.49 | A | C |
| ATOM | 1254 | CG | LEU | A | 442 | 54.042 | 29.552 | 100.679 | 1.00 | 30.92 | A | C |
| ATOM | 1255 | CD1 | LEU | A | 442 | 54.898 | 28.875 | 101.737 | 1.00 | 31.60 | A | C |
| ATOM | 1256 | CD2 | LEU | A | 442 | 54.863 | 29.836 | 99.434 | 1.00 | 31.89 | A | C |
| ATOM | 1257 | C | LEU | A | 442 | 50.877 | 28.009 | 98.984 | 1.00 | 28.59 | A | C |
| ATOM | 1258 | O | LEU | A | 442 | 51.084 | 27.420 | 97.929 | 1.00 | 28.30 | A | O |
| ATOM | 1259 | N | TYR | A | 443 | 49.896 | 27.674 | 99.821 | 1.00 | 30.49 | A | N |
| ATOM | 1260 | CA | TYR | A | 443 | 48.993 | 26.553 | 99.548 | 1.00 | 31.37 | A | C |
| ATOM | 1261 | CB | TYR | A | 443 | 49.181 | 25.450 | 100.601 | 1.00 | 30.85 | A | C |
| ATOM | 1262 | CG | TYR | A | 443 | 50.611 | 24.990 | 100.753 | 1.00 | 28.19 | A | C |
| ATOM | 1263 | CD1 | TYR | A | 443 | 51.462 | 25.575 | 101.697 | 1.00 | 25.52 | A | C |
| ATOM | 1264 | CE1 | TYR | A | 443 | 52.805 | 25.190 | 101.793 | 1.00 | 23.19 | A | C |
| ATOM | 1265 | CD2 | TYR | A | 443 | 51.133 | 24.007 | 99.913 | 1.00 | 27.48 | A | C |
| ATOM | 1266 | CE2 | TYR | A | 443 | 52.469 | 23.617 | 100.000 | 1.00 | 26.82 | A | C |
| ATOM | 1267 | CZ | TYR | A | 443 | 53.298 | 24.213 | 100.937 | 1.00 | 24.13 | A | C |
| ATOM | 1268 | OH | TYR | A | 443 | 54.618 | 23.837 | 100.986 | 1.00 | 24.42 | A | O |
| ATOM | 1269 | C | TYR | A | 443 | 47.518 | 26.945 | 99.497 | 1.00 | 31.76 | A | C |
| ATOM | 1270 | O | TYR | A | 443 | 46.719 | 26.112 | 99.036 | 1.00 | 33.61 | A | O |
| ATOM | 1271 | OT | TYR | A | 443 | 47.163 | 28.054 | 99.935 | 1.00 | 33.91 | A | O |
| ATOM | 2560 | ZN | ZN | A | 1 | 63.267 | 29.950 | 112.382 | 1.00 | 18.93 | Z | ZN |
| ATOM | 2561 | ZN | ZN | A | 2 | 70.729 | 19.617 | 111.673 | 1.00 | 22.07 | Z | ZN |
| ATOM | 2565 | CA | CA | A | 3 | 71.413 | 25.140 | 121.665 | 1.00 | 25.39 | C | C |
| ATOM | 2566 | CA | CA | A | 4 | 63.107 | 15.179 | 103.757 | 1.00 | 28.86 | C | C |
| ATOM | 2567 | CA | CA | A | 5 | 67.687 | 17.476 | 126.440 | 1.00 | 49.10 | C | C |
| ATOM | 2568 | CA | CA | A | 6 | 57.389 | 24.336 | 131.008 | 1.00 | 56.13 | C | C |
| ATOM | 2573 | CA | CA | A | 7 | 41.603 | 20.860 | 107.667 | 1.00 | 99.94 | C | C |
| ATOM | 2515 | C1 | FRA | A | 1 | 62.295 | 30.885 | 117.488 | 1.00 | 16.45 | M | C |
| ATOM | 2516 | C2 | FRA | A | 1 | 63.038 | 29.608 | 117.942 | 1.00 | 19.42 | M | C |
| ATOM | 2517 | C3 | FRA | A | 1 | 61.951 | 28.564 | 118.122 | 1.00 | 18.32 | M | C |
| ATOM | 2518 | C4 | FRA | A | 1 | 64.019 | 29.134 | 116.841 | 1.00 | 18.44 | M | C |
| ATOM | 2519 | C5 | FRA | A | 1 | 65.268 | 30.026 | 116.452 | 1.00 | 20.30 | M | C |
| ATOM | 2520 | C6 | FRA | A | 1 | 66.093 | 29.344 | 115.298 | 1.00 | 21.58 | M | C |
| ATOM | 2521 | N7 | FRA | A | 1 | 65.323 | 29.140 | 114.074 | 1.00 | 19.54 | M | N |
| ATOM | 2522 | O8 | FRA | A | 1 | 65.083 | 30.177 | 113.358 | 1.00 | 20.40 | M | O |
| ATOM | 2523 | C9 | FRA | A | 1 | 64.851 | 27.958 | 113.673 | 1.00 | 20.49 | M | C |
| ATOM | 2524 | O10 | FRA | A | 1 | 64.213 | 27.793 | 112.689 | 1.00 | 19.70 | M | O |
| ATOM | 2525 | C11 | FRA | A | 1 | 66.274 | 30.187 | 117.629 | 1.00 | 22.72 | M | C |
| ATOM | 2526 | N12 | FRA | A | 1 | 66.450 | 31.428 | 118.131 | 1.00 | 21.65 | M | N |
| ATOM | 2527 | C13 | FRA | A | 1 | 67.389 | 31.754 | 119.271 | 1.00 | 23.60 | M | C |
| ATOM | 2528 | C14 | FRA | A | 1 | 68.279 | 33.067 | 118.969 | 1.00 | 24.52 | M | C |
| ATOM | 2529 | C15 | FRA | A | 1 | 69.263 | 33.407 | 120.153 | 1.00 | 23.38 | M | C |
| ATOM | 2530 | C16 | FRA | A | 1 | 67.424 | 34.373 | 118.736 | 1.00 | 23.95 | M | C |
| ATOM | 2531 | C17 | FRA | A | 1 | 69.119 | 32.883 | 117.677 | 1.00 | 21.36 | M | C |
| ATOM | 2532 | C18 | FRA | A | 1 | 66.491 | 31.847 | 120.586 | 1.00 | 23.15 | M | C |
| ATOM | 2533 | O19 | FRA | A | 1 | 65.406 | 32.468 | 120.589 | 1.00 | 24.19 | M | O |
| ATOM | 2534 | N20 | FRA | A | 1 | 67.003 | 31.196 | 121.667 | 1.00 | 21.19 | M | N |
| ATOM | 2535 | C21 | FRA | A | 1 | 66.350 | 31.137 | 122.996 | 1.00 | 25.36 | M | C |
| ATOM | 2536 | O22 | FRA | A | 1 | 66.882 | 29.180 | 118.067 | 1.00 | 22.29 | M | O |
| ATOM | 1273 | CB | ASP | B | 110 | 39.115 | 26.293 | 159.737 | 1.00 | 61.05 | B | C |
| ATOM | 1274 | CG | ASP | B | 110 | 39.042 | 27.399 | 160.776 | 1.00 | 62.86 | B | C |
| ATOM | 1275 | OD1 | ASP | B | 110 | 40.101 | 27.786 | 161.322 | 1.00 | 62.33 | B | O |
| ATOM | 1276 | OD2 | ASP | B | 110 | 37.917 | 27.876 | 161.046 | 1.00 | 63.37 | B | O |
| ATOM | 1277 | C | ASP | B | 110 | 40.865 | 24.867 | 160.849 | 1.00 | 56.49 | B | C |
| ATOM | 1278 | O | ASP | B | 110 | 41.355 | 25.782 | 161.519 | 1.00 | 56.57 | B | O |
| ATOM | 1279 | N | ASP | B | 110 | 38.439 | 24.617 | 161.434 | 1.00 | 58.69 | B | N |
| ATOM | 1280 | CA | ASP | B | 110 | 39.418 | 24.920 | 160.351 | 1.00 | 58.71 | B | C |
| ATOM | 1281 | N | LEU | B | 111 | 41.541 | 23.778 | 160.512 | 1.00 | 52.64 | B | N |
| ATOM | 1282 | CA | LEU | B | 111 | 42.921 | 23.579 | 160.910 | 1.00 | 48.28 | B | C |
| ATOM | 1283 | CB | LEU | B | 111 | 43.265 | 22.094 | 160.794 | 1.00 | 48.88 | B | C |
| ATOM | 1284 | CG | LEU | B | 111 | 44.509 | 21.605 | 161.528 | 1.00 | 50.33 | B | C |
| ATOM | 1285 | CD1 | LEU | B | 111 | 44.535 | 22.156 | 162.948 | 1.00 | 50.73 | B | C |
| ATOM | 1286 | CD2 | LEU | B | 111 | 44.500 | 20.088 | 161.540 | 1.00 | 51.89 | B | C |
| ATOM | 1287 | C | LEU | B | 111 | 43.846 | 24.419 | 160.029 | 1.00 | 43.58 | B | C |
| ATOM | 1288 | O | LEU | B | 111 | 43.584 | 24.607 | 158.838 | 1.00 | 42.86 | B | O |
| ATOM | 1289 | N | LYS | B | 112 | 44.917 | 24.942 | 160.615 | 1.00 | 37.78 | B | N |
| ATOM | 1290 | CA | LYS | B | 112 | 45.858 | 25.745 | 159.846 | 1.00 | 34.84 | B | C |
| ATOM | 1291 | CB | LYS | B | 112 | 45.341 | 27.176 | 159.702 | 1.00 | 35.26 | B | C |
| ATOM | 1292 | CG | LYS | B | 112 | 45.439 | 28.007 | 160.972 | 1.00 | 39.03 | B | C |
| ATOM | 1293 | CD | LYS | B | 112 | 45.160 | 29.466 | 160.674 | 1.00 | 38.56 | B | C |
| ATOM | 1294 | CE | LYS | B | 112 | 45.529 | 30.353 | 161.845 | 1.00 | 41.04 | B | C |
| ATOM | 1295 | NZ | LYS | B | 112 | 45.396 | 31.790 | 161.475 | 1.00 | 41.35 | B | N |
| ATOM | 1296 | C | LYS | B | 112 | 47.233 | 25.767 | 160.498 | 1.00 | 32.44 | B | C |
| ATOM | 1297 | O | LYS | B | 112 | 47.395 | 25.317 | 161.628 | 1.00 | 31.94 | B | O |
| ATOM | 1298 | N | TRP | B | 113 | 48.225 | 26.284 | 159.779 | 1.00 | 30.35 | B | N |
| ATOM | 1299 | CA | TRP | B | 113 | 49.571 | 26.359 | 160.320 | 1.00 | 30.31 | B | C |
| ATOM | 1300 | CB | TRP | B | 113 | 50.613 | 26.516 | 159.204 | 1.00 | 27.93 | B | C |
| ATOM | 1301 | CG | TRP | B | 113 | 50.690 | 25.351 | 158.258 | 1.00 | 27.86 | B | C |
| ATOM | 1302 | CD2 | TRP | B | 113 | 51.204 | 24.040 | 158.541 | 1.00 | 26.96 | B | C |
| ATOM | 1303 | CE2 | TRP | B | 113 | 51.058 | 23.271 | 157.363 | 1.00 | 27.29 | B | C |
| ATOM | 1304 | CE3 | TRP | B | 113 | 51.773 | 23.441 | 159.674 | 1.00 | 23.04 | B | C |
| ATOM | 1305 | CD1 | TRP | B | 113 | 50.266 | 25.321 | 156.958 | 1.00 | 26.75 | B | C |
| ATOM | 1306 | NE1 | TRP | B | 113 | 50.483 | 24.078 | 156.415 | 1.00 | 23.61 | B | N |
| ATOM | 1307 | CZ2 | TRP | B | 113 | 51.460 | 21.930 | 157.287 | 1.00 | 24.46 | B | C |
| ATOM | 1308 | CZ3 | TRP | B | 113 | 52.171 | 22.108 | 159.597 | 1.00 | 23.32 | B | C |
| ATOM | 1309 | CH2 | TRP | B | 113 | 52.012 | 21.369 | 158.410 | 1.00 | 23.78 | B | C |
| ATOM | 1310 | C | TRP | B | 113 | 49.645 | 27.558 | 161.252 | 1.00 | 31.82 | B | C |
| ATOM | 1311 | O | TRP | B | 113 | 49.024 | 28.589 | 160.996 | 1.00 | 32.51 | B | O |
| ATOM | 1312 | N | HIS | B | 114 | 50.400 | 27.419 | 162.335 | 1.00 | 31.20 | B | N |
| ATOM | 1313 | CA | HIS | B | 114 | 50.549 | 28.507 | 163.285 | 1.00 | 31.82 | B | C |
| ATOM | 1314 | CB | HIS | B | 114 | 50.577 | 27.963 | 164.711 | 1.00 | 31.55 | B | C |
| ATOM | 1315 | CG | HIS | B | 114 | 49.237 | 27.513 | 165.193 | 1.00 | 32.54 | B | C |
| ATOM | 1316 | CD2 | HIS | B | 114 | 47.999 | 27.711 | 164.681 | 1.00 | 32.52 | B | C |
| ATOM | 1317 | ND1 | HIS | B | 114 | 49.064 | 26.767 | 166.337 | 1.00 | 33.18 | B | N |
| ATOM | 1318 | CE1 | HIS | B | 114 | 47.777 | 26.523 | 166.510 | 1.00 | 32.64 | B | C |
| ATOM | 1319 | NE2 | HIS | B | 114 | 47.110 | 27.085 | 165.518 | 1.00 | 34.29 | B | N |
| ATOM | 1320 | C | HIS | B | 114 | 51.774 | 29.357 | 163.006 | 1.00 | 30.66 | B | C |
| ATOM | 1321 | O | HIS | B | 114 | 51.997 | 30.358 | 163.679 | 1.00 | 34.99 | B | O |
| ATOM | 1322 | N | HIS | B | 115 | 52.572 | 28.950 | 162.022 | 1.00 | 28.55 | B | N |
| ATOM | 1323 | CA | HIS | B | 115 | 53.245 | 29.722 | 161.617 | 1.00 | 25.56 | B | C |
| ATOM | 1324 | CB | HIS | B | 115 | 55.044 | 28.934 | 161.824 | 1.00 | 25.76 | B | C |
| ATOM | 1325 | CG | HIS | B | 115 | 55.057 | 27.591 | 161.164 | 1.00 | 25.72 | B | C |
| ATOM | 1326 | CD2 | HIS | B | 115 | 55.839 | 27.090 | 160.180 | 1.00 | 24.52 | B | C |
| ATOM | 1327 | ND1 | HIS | B | 115 | 54.198 | 26.576 | 161.526 | 1.00 | 24.79 | B | N |
| ATOM | 1328 | CE1 | HIS | B | 115 | 54.453 | 25.506 | 160.796 | 1.00 | 22.93 | B | C |
| ATOM | 1329 | NE2 | HIS | B | 115 | 55.445 | 25.791 | 159.971 | 1.00 | 23.07 | B | N |
| ATOM | 1330 | C | HIS | B | 115 | 53.553 | 30.058 | 160.144 | 1.00 | 25.78 | B | C |
| ATOM | 1331 | O | HIS | B | 115 | 52.775 | 29.406 | 159.456 | 1.00 | 26.11 | B | O |
| ATOM | 1332 | N | HIS | B | 116 | 54.261 | 31.072 | 159.666 | 1.00 | 25.82 | B | N |
| ATOM | 1333 | CA | HIS | B | 116 | 54.133 | 31.514 | 158.286 | 1.00 | 25.15 | B | C |
| ATOM | 1334 | CB | HIS | B | 116 | 53.911 | 33.030 | 158.253 | 1.00 | 25.12 | B | C |
| ATOM | 1335 | CG | HIS | H | 116 | 52.559 | 33.446 | 158.740 | 1.00 | 26.86 | B | C |
| ATOM | 1336 | CD2 | HIS | B | 116 | 51.495 | 32.712 | 159.144 | 1.00 | 25.46 | B | C |
| ATOM | 1337 | ND1 | HIS | B | 116 | 52.177 | 34.766 | 158.840 | 1.00 | 26.98 | B | N |
| ATOM | 1338 | GE1 | HIS | B | 116 | 50.934 | 34.826 | 159.285 | 1.00 | 29.50 | B | C |
| ATOM | 1339 | NE2 | HIS | B | 116 | 50.498 | 33.594 | 159.477 | 1.00 | 28.22 | B | N |
| ATOM | 1340 | C | HIS | B | 116 | 55.290 | 31.152 | 157.372 | 1.00 | 24.84 | B | C |
| ATOM | 1341 | O | HIS | B | 116 | 55.163 | 31.228 | 156.155 | 1.00 | 24.51 | B | O |
| ATOM | 1342 | N | ASN | B | 117 | 56.421 | 30.779 | 157.955 | 1.00 | 23.92 | B | N |
| ATOM | 1343 | CA | ASN | B | 117 | 57.581 | 30.395 | 157.166 | 1.00 | 23.42 | B | C |
| ATOM | 1344 | CB | ASN | B | 117 | 58.868 | 30.914 | 157.824 | 1.00 | 24.61 | B | C |
| ATOM | 1345 | CG | ASN | B | 117 | 59.019 | 30.486 | 159.285 | 1.00 | 27.08 | B | C |
| ATOM | 1346 | OD1 | ASN | B | 117 | 58.047 | 30.395 | 160.041 | 1.00 | 28.75 | B | O |
| ATOM | 1347 | ND2 | ASN | B | 117 | 60.259 | 30.244 | 159.689 | 1.00 | 24.54 | B | N |
| ATOM | 1348 | C | ASN | B | 117 | 57.568 | 28.878 | 157.046 | 1.00 | 23.79 | B | C |
| ATOM | 1349 | O | ASN | B | 117 | 58.249 | 28.160 | 157.779 | 1.00 | 22.99 | B | O |
| ATOM | 1350 | N | ILE | B | 118 | 56.760 | 28.401 | 156.107 | 1.00 | 23.12 | B | N |
| ATOM | 1351 | CA | ILE | B | 118 | 56.585 | 26.979 | 155.876 | 1.00 | 24.05 | B | C |
| ATOM | 1352 | CB | ILE | B | 118 | 55.245 | 26.734 | 155.156 | 1.00 | 25.87 | B | C |
| ATOM | 1353 | CG2 | ILE | B | 118 | 55.066 | 25.256 | 154.855 | 1.00 | 25.54 | B | C |
| ATOM | 1354 | CG1 | ILE | B | 118 | 54.104 | 27.259 | 156.039 | 1.00 | 26.83 | B | C |
| ATOM | 1355 | CD1 | ILE | B | 118 | 52.721 | 27.053 | 155.465 | 1.00 | 31.75 | B | C |
| ATOM | 1356 | C | ILE | B | 118 | 57.722 | 26.340 | 155.097 | 1.00 | 23.08 | B | C |
| ATOM | 1357 | O | ILE | B | 118 | 58.319 | 26.962 | 154.223 | 1.00 | 25.00 | B | O |
| ATOM | 1358 | N | THR | B | 119 | 58.029 | 25.094 | 155.429 | 1.00 | 21.12 | B | N |
| ATOM | 1359 | CA | THR | B | 119 | 59.096 | 24.382 | 154.744 | 1.00 | 19.30 | B | C |
| ATOM | 1360 | CB | THR | B | 119 | 60.203 | 23.938 | 155.721 | 1.00 | 18.41 | B | C |
| ATOM | 1361 | OG1 | THR | B | 119 | 59.640 | 23.097 | 156.732 | 1.00 | 18.81 | B | O |
| ATOM | 1362 | CG2 | THR | B | 119 | 60.861 | 25.147 | 156.359 | 1.00 | 14.91 | B | C |
| ATOM | 1363 | C | THR | B | 119 | 58.564 | 23.156 | 154.026 | 1.00 | 18.87 | B | C |
| ATOM | 1364 | O | THR | B | 119 | 57.539 | 22.586 | 154.415 | 1.00 | 17.08 | B | O |
| ATOM | 1365 | N | TYR | B | 120 | 59.260 | 22.756 | 152.966 | 1.00 | 18.82 | B | N |
| ATOM | 1366 | CA | TYR | B | 120 | 58.848 | 21.584 | 152.207 | 1.00 | 17.71 | B | C |
| ATOM | 1367 | CB | TYR | B | 120 | 57.966 | 21.973 | 151.004 | 1.00 | 14.12 | B | C |
| ATOM | 1368 | CG | TYR | B | 120 | 58.671 | 22.725 | 149.891 | 1.00 | 14.85 | B | C |
| ATOM | 1369 | CD1 | TYR | B | 120 | 58.811 | 24.113 | 149.936 | 1.00 | 15.43 | B | C |
| ATOM | 1370 | CE1 | TYR | B | 120 | 59.392 | 24.817 | 148.882 | 1.00 | 12.87 | B | C |
| ATOM | 1371 | CD2 | TYR | B | 120 | 59.144 | 22.052 | 148.761 | 1.00 | 15.15 | B | C |
| ATOM | 1372 | CE2 | TYR | B | 120 | 59.725 | 22.746 | 147.702 | 1.00 | 15.56 | B | C |
| ATOM | 1373 | CZ | TYR | B | 120 | 59.842 | 24.126 | 147.766 | 1.00 | 17.67 | B | C |
| ATOM | 1374 | OH | TYR | B | 120 | 60.378 | 24.816 | 146.702 | 1.00 | 20.47 | B | O |
| ATOM | 1375 | C | TYR | B | 120 | 60.048 | 20.798 | 151.726 | 1.00 | 14.81 | B | C |
| ATOM | 1376 | O | TYR | B | 120 | 61.116 | 21.353 | 151.497 | 1.00 | 14.65 | B | O |
| ATOM | 1377 | N | TRP | B | 121 | 59.840 | 19.499 | 151.567 | 1.00 | 12.91 | B | N |
| ATOM | 1378 | CA | TRP | B | 121 | 60.867 | 18.587 | 151.121 | 1.00 | 14.27 | B | C |
| ATOM | 1379 | CB | TRP | B | 121 | 61.271 | 17.677 | 152.295 | 1.00 | 16.23 | B | C |
| ATOM | 1380 | CO | TRP | B | 121 | 62.237 | 16.559 | 151.970 | 1.00 | 16.27 | B | C |
| ATOM | 1381 | CD2 | TRP | B | 121 | 62.292 | 15.266 | 152.595 | 1.00 | 17.47 | B | C |
| ATOM | 1382 | CE2 | TRP | B | 121 | 63.424 | 14.600 | 152.078 | 1.00 | 17.45 | B | C |
| ATOM | 1383 | CE3 | TRP | B | 121 | 61.496 | 14.610 | 153.S48 | 1.00 | 17.63 | B | C |
| ATOM | 1384 | CD1 | TRP | B | 121 | 63.303 | 16.613 | 151.117 | 1.00 | 17.04 | B | C |
| ATOM | 1385 | NE1 | TRP | B | 121 | 64.021 | 15.442 | 151.179 | 1.00 | 16.74 | B | N |
| ATOM | 1386 | CZ2 | TRP | B | 121 | 63.783 | 13.307 | 152.484 | 1.00 | 18.60 | B | C |
| ATOM | 1387 | CZ3 | TRP | B | 121 | 61.853 | 13.327 | 153.952 | 1.00 | 18.18 | B | C |
| ATOM | 1388 | CH2 | TRP | B | 121 | 62.988 | 12.691 | 153.420 | 1.00 | 18.22 | B | C |
| ATOM | 1389 | C | TRP | B | 121 | 60.355 | 17.741 | 149.955 | 1.00 | 15.49 | B | C |
| ATOM | 1390 | O | TRP | B | 121 | 59.312 | 17.093 | 150.061 | 1.00 | 13.62 | B | O |
| ATOM | 1391 | N | ILE | B | 122 | 61.080 | 17.770 | 148.841 | 1.00 | 12.10 | B | N |
| ATOM | 1392 | CA | ILE | B | 122 | 60.729 | 16.956 | 147.696 | 1.00 | 13.59 | B | C |
| ATOM | 1393 | CB | ILE | B | 122 | 61.167 | 17.608 | 146.381 | 1.00 | 14.78 | B | C |
| ATOM | 1394 | CG2 | ILE | B | 122 | 60.773 | 16.715 | 145.214 | 1.00 | 10.29 | B | C |
| ATOM | 1395 | CO1 | ILE | B | 122 | 60.520 | 18.991 | 146.254 | 1.00 | 13.64 | B | C |
| ATOM | 1396 | CD1 | ILE | B | 122 | 61.024 | 19.811 | 145.080 | 1.00 | 12.29 | B | C |
| ATOM | 1397 | C | ILE | B | 122 | 61.514 | 15.672 | 147.918 | 1.00 | 13.64 | B | C |
| ATOM | 1398 | O | ILE | B | 122 | 62.694 | 15.588 | 147.592 | 1.00 | 14.54 | B | O |
| ATOM | 1399 | N | GLN | B | 123 | 60.855 | 14.679 | 148.496 | 1.00 | 13.68 | B | N |
| ATOM | 1400 | CA | GLN | B | 123 | 61.496 | 13.413 | 148.802 | 1.00 | 15.77 | B | C |
| ATOM | 1401 | CB | GLN | B | 123 | 60.564 | 12.573 | 149.662 | 1.00 | 14.28 | B | C |
| ATOM | 1402 | CG | GLN | B | 123 | 61.092 | 11.200 | 149.957 | 1.00 | 19.68 | B | C |
| ATOM | 1403 | CD | GLN | B | 123 | 60.133 | 10.402 | 150.794 | 1.00 | 22.52 | B | C |
| ATOM | 1404 | OE1 | GLN | B | 123 | 59.933 | 9.211 | 150.560 | 1.00 | 28.95 | B | O |
| ATOM | 1405 | NE2 | GLN | B | 123 | 59.534 | 11.048 | 151.782 | 1.00 | 19.79 | B | N |
| ATOM | 1406 | C | GLN | B | 123 | 61.971 | 12.587 | 147.604 | 1.00 | 18.55 | B | C |
| ATOM | 1407 | O | GLN | B | 123 | 63.086 | 12.069 | 147.611 | 1.00 | 18.76 | B | O |
| ATOM | 1408 | N | ASN | B | 124 | 61.118 | 12.428 | 146.596 | 1.00 | 19.57 | B | N |
| ATOM | 1409 | CA | ASN | B | 124 | 61.492 | 11.664 | 145.412 | 1.00 | 17.91 | B | C |
| ATOM | 1410 | CB | ASN | B | 124 | 61.111 | 10.184 | 145.549 | 1.00 | 17.29 | B | C |
| ATOM | 1411 | CG | ASN | B | 124 | 59.628 | 9.965 | 145.734 | 1.00 | 18.23 | B | C |
| ATOM | 1412 | OD1 | ASN | B | 124 | 58.830 | 10.882 | 145.602 | 1.00 | 20.83 | B | O |
| ATOM | 1413 | ND2 | ASN | B | 124 | 59.254 | 8.735 | 146.035 | 1.00 | 18.51 | B | N |
| ATOM | 1414 | C | ASN | B | 124 | 60.868 | 12.265 | 144.165 | 1.00 | 19.24 | B | C |
| ATOM | 1415 | O | ASN | B | 124 | 60.138 | 13.261 | 144.239 | 1.00 | 19.36 | B | O |
| ATOM | 1416 | N | TYR | B | 125 | 61.164 | 11.666 | 143.016 | 1.00 | 18.73 | B | N |
| ATOM | 1417 | CA | TYR | B | 125 | 60.670 | 12.187 | 141.749 | 1.00 | 14.58 | B | C |
| ATOM | 1418 | CB | TYR | B | 125 | 61.844 | 12.731 | 140.921 | 1.00 | 15.39 | B | C |
| ATOM | 1419 | CG | TYR | B | 125 | 62.538 | 13.932 | 141.536 | 1.00 | 13.70 | B | C |
| ATOM | 1420 | CD1 | TYR | B | 125 | 63.526 | 13.777 | 142.510 | 1.00 | 12.60 | B | C |
| ATOM | 1421 | CE1 | TYR | B | 125 | 64.113 | 14.889 | 143.124 | 1.00 | 11.44 | B | C |
| ATOM | 1422 | CD2 | TYR | B | 125 | 62.156 | 15.229 | 141.187 | 1.00 | 15.12 | B | C |
| ATOM | 1423 | CE2 | TYR | B | 125 | 62.731 | 16.341 | 141.793 | 1.00 | 11.78 | B | C |
| ATOM | 1424 | CZ | TYR | B | 125 | 63.706 | 16.164 | 142.760 | 1.00 | 13.63 | B | C |
| ATOM | 1425 | OH | TYR | B | 125 | 64.257 | 17.270 | 143.365 | 1.00 | 11.62 | B | O |
| ATOM | 1426 | C | TYR | B | 125 | 59.890 | 11.203 | 140.899 | 1.00 | 14.07 | B | C |
| ATOM | 1427 | O | TYR | B | 125 | 59.880 | 10.005 | 141.158 | 1.00 | 12.64 | B | O |
| ATOM | 1428 | N | SER | B | 126 | 59.230 | 11.737 | 139.877 | 1.00 | 15.52 | B | N |
| ATOM | 1429 | CA | SER | B | 126 | 58.461 | 10.929 | 138.936 | 1.00 | 17.11 | B | C |
| ATOM | 1430 | CB | SER | B | 126 | 57.246 | 11.705 | 138.419 | 1.00 | 17.11 | B | C |
| ATOM | 1431 | OG | SER | B | 126 | 56.716 | 11.100 | 137.252 | 1.00 | 14.91 | B | O |
| ATOM | 1432 | C | SER | B | 126 | 59.374 | 10.596 | 137.766 | 1.00 | 20.19 | B | C |
| ATOM | 1433 | O | SER | B | 126 | 60.298 | 11.345 | 137.452 | 1.00 | 19.00 | B | O |
| ATOM | 1434 | N | GLU | B | 127 | 59.109 | 9.480 | 137.108 | 1.00 | 23.95 | B | N |
| ATOM | 1435 | CA | GLU | B | 127 | 59.937 | 9.079 | 135.982 | 1.00 | 27.98 | B | C |
| ATOM | 1436 | CB | GLU | B | 127 | 59.795 | 7.575 | 135.743 | 1.00 | 33.47 | B | C |
| ATOM | 1437 | CG | GLU | B | 127 | 59.913 | 6.740 | 137.004 | 1.00 | 40.62 | B | C |
| ATOM | 1438 | CD | GLU | B | 127 | 60.077 | 5.255 | 136.718 | 1.00 | 45.97 | B | C |
| ATOM | 1439 | OE1 | GLU | B | 127 | 61.082 | 4.880 | 136.068 | 1.00 | 47.09 | B | O |
| ATOM | 1440 | OE2 | GLU | B | 127 | 59.204 | 4.466 | 137.148 | 1.00 | 47.02 | B | O |
| ATOM | 1441 | C | GLU | B | 127 | 59.585 | 9.825 | 134.700 | 1.00 | 26.19 | B | C |
| ATOM | 1442 | O | GLU | B | 127 | 60.331 | 9.763 | 133.732 | 1.00 | 27.48 | B | O |
| ATOM | 1443 | N | ASP | B | 128 | 58.464 | 10.543 | 134.702 | 1.00 | 23.99 | B | N |
| ATOM | 1444 | CA | ASP | B | 128 | 58.008 | 11.256 | 133.511 | 1.00 | 21.74 | B | C |
| ATOM | 1445 | CB | ASP | B | 128 | 56.496 | 11.480 | 133.557 | 1.00 | 22.01 | B | C |
| ATOM | 1446 | CG | ASP | B | 128 | 55.717 | 10.228 | 133.884 | 1.00 | 18.40 | B | C |
| ATOM | 1447 | OD1 | ASP | B | 128 | 56.138 | 9.119 | 133.514 | 1.00 | 21.04 | B | O |
| ATOM | 1448 | OD2 | ASP | B | 128 | 54.652 | 10.371 | 134.504 | 1.00 | 17.97 | B | O |
| ATOM | 1449 | C | ASP | B | 128 | 58.637 | 12.606 | 133.194 | 1.00 | 22.03 | B | C |
| ATOM | 1450 | O | ASP | B | 128 | 58.578 | 13.052 | 132.0Sf | 1.00 | 21.61 | B | O |
| ATOM | 1451 | N | LEU | B | 129 | 59.207 | 13.273 | 134.189 | 1.00 | 19.22 | B | N |
| ATOM | 1452 | CA | LEU | B | 129 | 59.797 | 14.582 | 133.953 | 1.00 | 17.22 | B | C |
| ATOM | 1453 | CB | LEU | B | 129 | 58.887 | 15.664 | 134.528 | 1.00 | 16.52 | B | C |
| ATOM | 1454 | CG | LEU | B | 129 | 57.497 | 15.796 | 133.901 | 1.00 | 20.26 | B | C |
| ATOM | 1455 | CD1 | LEU | B | 129 | 56.642 | 16.745 | 134.732 | 1.00 | 18.39 | B | C |
| ATOM | 1456 | CD2 | LEU | B | 129 | 57.629 | 16.303 | 132.471 | 1.00 | 19.31 | B | C |
| ATOM | 1457 | C | LEU | B | 129 | 61.179 | 14.713 | 134.566 | 1.00 | 17.81 | B | C |
| ATOM | 1458 | O | LEU | B | 129 | 61.515 | 14.000 | 135.511 | 1.00 | 20.83 | B | O |
| ATOM | 1459 | N | PRO | B | 130 | 62.014 | 15.617 | 134.021 | 1.00 | 17.25 | B | N |
| ATOM | 1460 | CD | PRO | B | 130 | 61.811 | 16.408 | 132.796 | 1.00 | 15.36 | B | C |
| ATOM | 1461 | CA | PRO | B | 130 | 63.371 | 15.820 | 134.557 | 1.00 | 17.25 | B | C |
| ATOM | 1462 | CB | PRO | B | 130 | 64.002 | 16.790 | 133.554 | 1.00 | 14.93 | B | C |
| ATOM | 1463 | CG | PRO | B | 130 | 63.214 | 16.559 | 132.292 | 1.00 | 15.96 | B | C |
| ATOM | 1464 | C | PRO | B | 130 | 63.250 | 16.450 | 135.954 | 1.00 | 15.64 | B | C |
| ATOM | 1465 | O | PRO | B | 130 | 62.245 | 17.096 | 136.250 | 1.00 | 15.44 | B | O |
| ATOM | 1466 | N | ARG | B | 131 | 64.259 | 16.265 | 136.805 | 1.00 | 16.42 | B | N |
| ATOM | 1467 | CA | ARG | B | 131 | 64.237 | 16.840 | 138.159 | 1.00 | 16.86 | B | C |
| ATOM | 1468 | CB | ARG | B | 131 | 65.551 | 16.591 | 138.906 | 1.00 | 18.20 | B | C |
| ATOM | 1469 | CG | ARG | B | 131 | 65.708 | 15.207 | 139.469 | 1.00 | 22.57 | B | C |
| ATOM | 1470 | CD | ARG | B | 131 | 66.845 | 15.143 | 140.470 | 1.00 | 21.69 | B | C |
| ATOM | 1471 | NE | ARG | B | 131 | 67.022 | 13.771 | 140.932 | 1.00 | 26.97 | B | N |
| ATOM | 1472 | CZ | ARG | B | 131 | 67.541 | 13.427 | 142.107 | 1.00 | 28.81 | B | C |
| ATOM | 1473 | NH1 | ARG | B | 131 | 67.945 | 14.360 | 142.966 | 1.00 | 24.33 | B | N |
| ATOM | 1474 | NH2 | ARG | B | 131 | 67.641 | 12.143 | 142.424 | 1.00 | 28.78 | B | N |
| ATOM | 1475 | C | ARG | B | 131 | 64.022 | 18.338 | 138.138 | 1.00 | 16.07 | B | C |
| ATOM | 1476 | O | ARG | B | 131 | 63.219 | 18.869 | 138.899 | 1.00 | 15.45 | B | O |
| ATOM | 1477 | N | ALA | B | 132 | 64.764 | 19.013 | 137.268 | 1.00 | 14.39 | B | N |
| ATOM | 1478 | CA | ALA | B | 132 | 64.685 | 20.461 | 137.163 | 1.00 | 16.40 | B | C |
| ATOM | 1479 | CB | ALA | B | 132 | 65.677 | 20.959 | 136.106 | 1.00 | 17.38 | B | C |
| ATOM | 1480 | C | ALA | B | 132 | 63.278 | 20.946 | 136.837 | 1.00 | 16.67 | B | C |
| ATOM | 1481 | O | ALA | B | 132 | 62.844 | 21.984 | 137.326 | 1.00 | 18.93 | B | O |
| ATOM | 1482 | N | VAL | B | 133 | 62.566 | 20.192 | 136.008 | 1.00 | 15.47 | B | N |
| ATOM | 1483 | CA | VAL | B | 133 | 61.213 | 20.562 | 135.626 | 1.00 | 13.45 | B | C |
| ATOM | 1484 | CB | VAL | B | 133 | 60.721 | 19.694 | 134.433 | 1.00 | 14.12 | B | C |
| ATOM | 1485 | CG1 | VAL | B | 133 | 59.229 | 19.926 | 134.177 | 1.00 | 9.73 | B | C |
| ATOM | 1486 | CO2 | VAL | B | 133 | 61.530 | 20.037 | 133.190 | 1.00 | 12.36 | B | C |
| ATOM | 1487 | C | VAL | B | 133 | 60.259 | 20.394 | 136.803 | 1.00 | 14.22 | B | C |
| ATOM | 1488 | O | VAL | B | 133 | 59.362 | 21.211 | 137.016 | 1.00 | 13.15 | B | O |
| ATOM | 1489 | N | ILE | B | 134 | 60.460 | 19.323 | 137.563 | 1.00 | 15.06 | B | N |
| ATOM | 1490 | CA | ILE | B | 134 | 59.617 | 19.029 | 138.709 | 1.00 | 13.17 | B | C |
| ATOM | 1491 | GB | ILE | B | 134 | 59.855 | 17.580 | 139.200 | 1.00 | 14.87 | B | C |
| ATOM | 1492 | CG2 | ILE | B | 134 | 59.229 | 17.371 | 140.571 | 1.00 | 14.89 | B | C |
| ATOM | 1493 | CG1 | ILE | B | 134 | 59.287 | 16.597 | 138.166 | 1.00 | 11.36 | B | C |
| ATOM | 1494 | CD1 | ILE | B | 134 | 59.476 | 15.131 | 138.521 | 1.00 | 11.57 | B | C |
| ATOM | 1495 | C | ILE | B | 134 | 59.862 | 20.025 | 139.830 | 1.00 | 15.99 | B | C |
| ATOM | 1496 | O | ILE | B | 134 | 58.908 | 20.543 | 140.404 | 1.00 | 16.24 | B | O |
| ATOM | 1497 | N | ASP | B | 135 | 61.132 | 20.308 | 140.134 | 1.00 | 15.66 | B | N |
| ATOM | 1498 | CA | ASP | B | 135 | 61.447 | 21.279 | 141.184 | 1.00 | 15.15 | B | C |
| ATOM | 1499 | CB | ASP | B | 135 | 62.959 | 21.544 | 141.288 | 1.00 | 12.56 | B | C |
| ATOM | 1SOO | CG | ASP | B | 135 | 63.746 | 20.351 | 141.803 | 1.00 | 10.35 | B | C |
| ATOM | 1501 | OD1 | ASP | B | 135 | 63.156 | 19.399 | 142.353 | 1.00 | 11.63 | B | O |
| ATOM | 1502 | OD2 | ASP | B | 135 | 64.983 | 20.371 | 141.664 | 1.00 | 17.25 | B | O |
| ATOM | 1503 | C | ASP | B | 135 | 60.752 | 22.603 | 140.871 | 1.00 | 15.48 | B | C |
| ATOM | 1504 | O | ASP | B | 135 | 60.095 | 23.187 | 141.732 | 1.00 | 20.45 | B | O |
| ATOM | 1505 | N | ASP | B | 136 | 60.902 | 23.069 | 139.635 | 1.00 | 14.76 | B | N |
| ATOM | 1506 | CA | ASP | B | 136 | 60.313 | 24.328 | 139.189 | 1.00 | 13.88 | B | C |
| ATOM | 1507 | CB | ASP | B | 136 | 60.768 | 24.621 | 137.762 | 1.00 | 16.77 | B | C |
| ATOM | 1508 | CG | ASP | B | 136 | 60.207 | 25.919 | 137.221 | 1.00 | 19.24 | B | C |
| ATOM | 1509 | OD1 | ASP | B | 136 | 60.611 | 26.985 | 137.721 | 1.00 | 20.16 | B | O |
| ATOM | 1510 | OD2 | ASP | B | 136 | 59.362 | 25.870 | 136.297 | 1.00 | 18.28 | B | O |
| ATOM | 1511 | C | ASP | B | 136 | 58.785 | 24.354 | 139.267 | 1.00 | 13.68 | B | C |
| ATOM | 1512 | O | ASP | B | 136 | 58.200 | 25.343 | 139.697 | 1.00 | 18.58 | B | O |
| ATOM | 1513 | N | ALA | B | 137 | 58.135 | 23.276 | 138.850 | 1.00 | 12.86 | B | N |
| ATOM | 1514 | CA | ALA | B | 137 | 56.679 | 23.204 | 138.907 | 1.00 | 10.91 | B | C |
| ATOM | 1515 | CB | ALA | B | 137 | 56.196 | 21.882 | 138.315 | 1.00 | 9.57 | B | C |
| ATOM | 1516 | C | ALA | B | 137 | 56.174 | 23.343 | 140.347 | 1.00 | 13.69 | B | C |
| ATOM | 1517 | O | ALA | B | 137 | 55.184 | 24.034 | 140.601 | 1.00 | 14.16 | B | O |
| ATOM | 1518 | N | PHE | B | 138 | 56.831 | 22.668 | 141.289 | 1.00 | 15.83 | B | N |
| ATOM | 1519 | CA | PHE | B | 138 | 56.425 | 22.758 | 142.694 | 1.00 | 16.29 | B | C |
| ATOM | 1520 | CB | PHE | B | 138 | 57.182 | 21.737 | 143.559 | 1.00 | 13.24 | B | C |
| ATOM | 1521 | CG | PHE | B | 138 | 56.731 | 20.304 | 143.363 | 1.00 | 12.26 | B | C |
| ATOM | 1522 | CD1 | PHE | B | 138 | 55.409 | 20.007 | 143.025 | 1.00 | 13.72 | B | C |
| ATOM | 1523 | CD2 | PHE | B | 138 | 57.628 | 19.251 | 143.516 | 1.00 | 9.81 | B | C |
| ATOM | 1524 | CE1 | PHE | B | 138 | 54.995 | 18.679 | 142.841 | 1.00 | 13.46 | B | C |
| ATOM | 1525 | CE2 | PHE | B | 138 | 57.224 | 17.927 | 143.337 | 1.00 | 7.35 | B | C |
| ATOM | 1526 | CZ | PHE | B | 138 | 55.907 | 17.641 | 142.998 | 1.00 | 11.80 | B | C |
| ATOM | 1527 | C | PHE | B | 138 | 56.670 | 24.172 | 143.226 | 1.00 | 16.78 | B | C |
| ATOM | 1528 | O | PHE | B | 138 | 55.871 | 24.709 | 143.990 | 1.00 | 19.28 | B | O |
| ATOM | 1529 | N | ALA | B | 139 | 57.772 | 24.783 | 142.807 | 1.00 | 17.38 | B | N |
| ATOM | 1530 | CA | ALA | B | 139 | 58.093 | 26.136 | 143.246 | 1.00 | 17.57 | B | C |
| ATOM | 1531 | CB | ALA | B | 139 | 59.490 | 26.521 | 142.770 | 1.00 | 14.41 | B | C |
| ATOM | 1532 | C | ALA | B | 139 | 57.061 | 27.149 | 142.738 | 1.00 | 18.95 | B | C |
| ATOM | 1533 | O | ALA | B | 139 | 56.664 | 28.060 | 143.467 | 1.00 | 19.80 | B | O |
| ATOM | 1534 | N | ARG | B | 140 | 56.628 | 26.993 | 141.490 | 1.00 | 19.15 | B | N |
| ATOM | 1535 | CA | ARG | B | 140 | 55.642 | 27.906 | 140.917 | 1.00 | 17.98 | B | C |
| ATOM | 1536 | CB | ARG | B | 140 | 55.596 | 27.761 | 139.388 | 1.00 | 16.03 | B | C |
| ATOM | 1537 | CG | ARG | B | 140 | 56.888 | 28.201 | 138.707 | 1.00 | 16.18 | B | C |
| ATOM | 1538 | CD | ARG | B | 140 | 56.812 | 28.138 | 137.181 | 1.00 | 18.32 | B | C |
| ATOM | 1539 | NE | ARG | B | 140 | 56.746 | 26.768 | 136.685 | 1.00 | 14.27 | B | N |
| ATOM | 1540 | CZ | ARG | B | 140 | 55.666 | 26.213 | 136.150 | 1.00 | 14.50 | B | C |
| ATOM | 1541 | NE1 | ARG | B | 140 | 54.548 | 26.912 | 136.030 | 1.00 | 12.39 | B | N |
| ATOM | 1542 | NH2 | ARG | B | 140 | 55.701 | 24.946 | 135.753 | 1.00 | 14.50 | B | N |
| ATQM | 1543 | C | ARG | B | 140 | 54.274 | 27.638 | 141.531 | 1.00 | 18.29 | B | C |
| ATOM | 1544 | O | ARG | B | 140 | 53.451 | 28.544 | 141.661 | 1.00 | 22.02 | B | O |
| ATOM | 1545 | N | ALA | B | 141 | 54.036 | 26.392 | 141.927 | 1.00 | 17.80 | B | N |
| ATOM | 1546 | CA | ALA | B | 141 | 52.768 | 26.030 | 142.552 | 1.00 | 17.21 | B | C |
| ATOM | 1547 | CB | ALA | B | 141 | 52.656 | 24.520 | 142.660 | 1.00 | 17.90 | B | C |
| ATOM | 1548 | C | ALA | B | 141 | 52.660 | 26.677 | 143.941 | 1.00 | 19.87 | B | C |
| ATOM | 1549 | O | ALA | B | 141 | 51.572 | 27.056 | 144.369 | 1.00 | 19.56 | B | O |
| ATOM | 1550 | N | PHE | B | 142 | 53.787 | 26.795 | 144.645 | 1.00 | 20.99 | B | N |
| ATOM | 1551 | CA | PHE | B | 142 | 53.798 | 27.427 | 145.970 | 1.00 | 21.67 | B | C |
| ATOM | 1552 | CB | PHE | B | 142 | 55.072 | 27.069 | 146.759 | 1.00 | 18.85 | B | C |
| ATOM | 1553 | CG | PHE | B | 142 | 55.008 | 25.734 | 147.446 | 1.00 | 16.57 | B | C |
| ATOM | 1554 | CD1 | PHE | B | 142 | 53.976 | 25.449 | 148.333 | 1.00 | 14.21 | B | C |
| ATOM | 1555 | CD2 | PHE | B | 142 | 55.974 | 24.764 | 147.204 | 1.00 | 14.86 | B | C |
| ATOM | 1556 | CE1 | PHE | B | 142 | 53.904 | 24.220 | 148.972 | 1.00 | 18.27 | B | C |
| ATOM | 1557 | CE2 | PHE | B | 142 | 55.917 | 23.527 | 147.836 | 1.00 | 19.57 | B | C |
| ATOM | 1558 | CZ | PHE | B | 142 | 54.879 | 23.251 | 148.724 | 1.00 | 20.55 | B | C |
| ATOM | 1559 | C | PHE | B | 142 | 53.719 | 28.942 | 145.832 | 1.00 | 21.95 | B | C |
| ATOM | 1560 | O | PHE | B | 142 | 53.101 | 29.613 | 146.652 | 1.00 | 25.86 | B | O |
| ATOM | 1561 | N | ALA | B | 143 | 54.356 | 29.481 | 144.802 | 1.00 | 20.87 | B | N |
| ATOM | 1562 | CA | ALA | B | 143 | 54.344 | 30.921 | 144.576 | 1.00 | 21.45 | B | C |
| ATOM | 1563 | CB | ALA | B | 143 | 55.222 | 31.268 | 143.390 | 1.00 | 17.43 | B | C |
| ATOM | 1564 | C | ALA | B | 143 | 52.930 | 31.466 | 144.360 | 1.00 | 23.06 | B | C |
| ATOM | 1565 | O | ALA | B | 143 | 52.661 | 32.629 | 144.658 | 1.00 | 25.48 | B | O |
| ATOM | 1566 | N | LEU | B | 144 | 52.029 | 30.636 | 143.841 | 1.00 | 24.74 | B | N |
| ATOM | 1567 | CA | LEU | B | 144 | 50.652 | 31.075 | 143.615 | 1.00 | 26.04 | D | C |
| ATOM | 1568 | CB | LEU | B | 144 | 49.797 | 29.958 | 143.010 | 1.00 | 24.21 | B | C |
| ATOM | 1569 | CG | LEU | B | 144 | 49.951 | 29.560 | 141.539 | 1.00 | 27.50 | B | C |
| ATOM | 1570 | CD1 | LEU | B | 144 | 48.945 | 28.468 | 141.230 | 1.00 | 25.16 | B | C |
| ATOM | 1571 | CD2 | LEU | B | 144 | 49.718 | 30.754 | 140.630 | 1.00 | 23.79 | B | C |
| ATOM | 1572 | C | LEU | B | 144 | 50.020 | 31.481 | 144.934 | 1.00 | 28.47 | B | C |
| ATOM | 1573 | O | LEU | B | 144 | 49.343 | 32.500 | 145.030 | 1.00 | 30.66 | B | O |
| ATOM | 1574 | N | TRP | B | 145 | 50.245 | 30.664 | 145.951 | 1.00 | 28.49 | B | N |
| ATOM | 1575 | CA | TRP | B | 145 | 49.680 | 30.913 | 147.265 | 1.00 | 29.54 | B | C |
| ATOM | 1576 | CB | TRP | B | 145 | 49.652 | 29.609 | 148.053 | 1.00 | 26.39 | B | C |
| ATOM | 1577 | CG | TRP | B | 145 | 48.796 | 28.553 | 147.417 | 1.00 | 24.57 | B | C |
| ATOM | 1578 | CD2 | TRP | B | 145 | 47.366 | 28.550 | 147.330 | 1.00 | 25.64 | B | C |
| ATOM | 1579 | CE2 | TRP | B | 145 | 46.986 | 27.320 | 146.747 | 1.00 | 24.80 | B | C |
| ATOM | 1580 | CE3 | TRP | B | 145 | 46.366 | 29.464 | 147.695 | 1.00 | 25.42 | B | C |
| ATOM | 1581 | CD1 | TRP | B | 145 | 49.214 | 27.369 | 146.885 | 1.00 | 22.40 | B | C |
| ATOM | 1582 | NE1 | TRP | B | 145 | 48.133 | 26.619 | 146.483 | 1.00 | 22.24 | B | N |
| ATOM | 1583 | CZ2 | TRP | B | 145 | 45.644 | 26.977 | 146.522 | 1.00 | 27.07 | B | C |
| ATOM | 1584 | CZ3 | TRP | B | 145 | 45.029 | 29.122 | 147.471 | 1.00 | 26.11 | B | C |
| ATOM | 1585 | CH2 | TRP | B | 145 | 44.684 | 27.889 | 146.892 | 1.00 | 24.97 | B | C |
| ATOM | 1586 | C | TRP | B | 145 | 50.397 | 31.995 | 148.064 | 1.00 | 28.97 | B | C |
| ATOM | 1587 | O | TRY | B | 145 | 49.752 | 32.844 | 148.674 | 1.00 | 29.64 | B | O |
| ATOM | 1588 | N | SER | B | 146 | 51.724 | 31.979 | 148.063 | 1.00 | 30.94 | B | N |
| ATOM | 1589 | CA | SER | B | 146 | 52.474 | 32.986 | 148.808 | 1.00 | 33.11 | B | C |
| ATOM | 1590 | CB | SER | B | 146 | 53.967 | 32.675 | 148.776 | 1.00 | 31.16 | B | C |
| ATOM | 1591 | OG | SER | B | 146 | 54.465 | 32.773 | 147.457 | 1.00 | 34.33 | B | O |
| ATOM | 1592 | C | SER | B | 146 | 52.241 | 34.372 | 148.221 | 1.00 | 35.39 | B | C |
| ATOM | 1593 | O | SER | B | 146 | 52.600 | 35.381 | 148.827 | 1.00 | 38.15 | B | O |
| ATOM | 1594 | N | ALA | B | 147 | 51.641 | 34.418 | 147.039 | 1.00 | 35.46 | B | N |
| ATOM | 1595 | CA | ALA | B | 147 | 51.379 | 35.683 | 146.374 | 1.00 | 35.64 | B | C |
| ATOM | 1596 | CB | ALA | B | 147 | 51.187 | 35.457 | 144.883 | 1.00 | 35.78 | B | C |
| ATOM | 1597 | C | ALA | B | 147 | 50.160 | 36.380 | 146.949 | 1.00 | 36.30 | B | C |
| ATOM | 1598 | O | ALA | B | 147 | 50.054 | 37.600 | 146.875 | 1.00 | 35.93 | B | O |
| ATOM | 1599 | N | VAL | B | 148 | 49.238 | 35.611 | 147.519 | 1.00 | 36.26 | B | N |
| ATOM | 1600 | CA | VAL | B | 148 | 48.031 | 36.196 | 148.089 | 1.00 | 37.29 | B | C |
| ATOM | 1601 | GB | VAL | B | 148 | 46.761 | 35.601 | 147.435 | 1.00 | 37.45 | B | C |
| ATOM | 1602 | CG1 | VAL | B | 148 | 46.728 | 35.952 | 145.959 | 1.00 | 39.35 | B | C |
| ATOM | 1603 | CG2 | VAL | B | 148 | 46.735 | 34.099 | 147.614 | 1.00 | 37.42 | B | C |
| ATOM | 1604 | C | VAL | B | 148 | 47.926 | 36.035 | 149.606 | 1.00 | 37.54 | B | C |
| ATOM | 1605 | O | VAL | B | 148 | 46.838 | 36.144 | 150.173 | 1.00 | 38.76 | B | O |
| ATOM | 1606 | N | THR | B | 149 | 49.057 | 35.774 | 150.257 | 1.00 | 36.72 | B | N |
| ATOM | 1607 | CA | THR | B | 149 | 49.098 | 35.611 | 151.711 | 1.00 | 34.50 | B | C |
| ATOM | 1608 | GB | THR | B | 149 | 48.815 | 34.159 | 152.139 | 1.00 | 32.69 | B | C |
| ATOM | 1609 | OG1 | THR | B | 149 | 49.872 | 33.313 | 151.672 | 1.00 | 34.10 | B | O |
| ATOM | 1610 | CG2 | THR | B | 149 | 47.492 | 33.677 | 151.587 | 1.00 | 33.09 | B | C |
| ATOM | 1611 | C | THR | B | 149 | 50.484 | 35.975 | 152.241 | 1.00 | 34.43 | B | C |
| ATOM | 1612 | O | THR | B | 149 | 51.424 | 36.166 | 151.466 | 1.00 | 33.86 | B | O |
| ATOM | 1613 | N | PRO | B | 150 | 50.624 | 36.084 | 153.576 | 1.00 | 35.88 | B | N |
| ATOM | 1614 | CD | PRO | B | 150 | 49.542 | 36.096 | 154.582 | 1.00 | 36.97 | B | C |
| ATOM | 1615 | CA | PRO | B | 150 | 51.909 | 36.423 | 154.199 | 1.00 | 35.29 | B | C |
| ATOM | 1616 | GB | PRO | B | 150 | 51.491 | 36.993 | 155.548 | 1.00 | 33.79 | B | C |
| ATOM | 1617 | CG | PRO | B | 150 | 50.313 | 36.136 | 155.894 | 1.00 | 36.98 | B | C |
| ATOM | 1618 | C | PRO | B | 150 | 52.810 | 35.190 | 154.341 | 1.00 | 33.55 | B | C |
| ATOM | 1619 | O | PRO | B | 150 | 53.889 | 35.259 | 154.928 | 1.00 | 33.85 | B | O |
| ATOM | 1620 | N | LEU | B | 151 | 52.357 | 34.064 | 153.796 | 1.00 | 32.24 | B | N |
| ATOM | 1621 | CA | LEU | B | 151 | 53.111 | 32.813 | 153.850 | 1.00 | 29.72 | B | C |
| ATOM | 1622 | CB | LEU | B | 151 | 52.210 | 31.634 | 153.485 | 1.00 | 28.34 | B | C |
| ATOM | 1623 | CG | LEU | B | 151 | 50.920 | 31.386 | 154.263 | 1.00 | 29.72 | B | C |
| ATOM | 1624 | CD1 | LEU | B | 151 | 50.115 | 30.308 | 153.547 | 1.00 | 25.73 | B | C |
| ATOM | 1625 | CD2 | LEU | B | 151 | 51.241 | 30.971 | 155.697 | 1.00 | 27.68 | B | C |
| ATOM | 1626 | C | LEU | B | 151 | 54.301 | 32.796 | 152.897 | 1.00 | 28.80 | B | C |
| ATOM | 1627 | O | LEU | B | 151 | 54.253 | 33.384 | 151.817 | 1.00 | 29.79 | B | O |
| ATOM | 1628 | N | THR | B | 152 | 55.370 | 32.123 | 153.307 | 1.00 | 26.47 | B | N |
| ATOM | 1629 | CA | THR | B | 152 | 56.550 | 31.968 | 152.468 | 1.00 | 26.48 | B | C |
| ATOM | 1630 | GB | THR | B | 152 | 57.786 | 32.697 | 153.018 | 1.00 | 25.05 | B | C |
| ATOM | 1631 | OG1 | THR | B | 152 | 58.199 | 32.092 | 154.250 | 1.00 | 26.56 | B | O |
| ATOM | 1632 | CG2 | THR | B | 152 | 57.478 | 34.161 | 153.235 | 1.00 | 26.91 | B | C |
| ATOM | 1633 | C | THR | B | 152 | 56.822 | 30.469 | 152.444 | 1.00 | 27.63 | B | C |
| ATOM | 1634 | O | THR | B | 152 | 56.361 | 29.731 | 153.321 | 1.00 | 28.65 | B | O |
| ATOM | 1635 | N | PHE | B | 153 | 57.561 | 30.020 | 151.438 | 1.00 | 26.34 | B | N |
| ATOM | 1636 | CA | PHE | B | 153 | 57.858 | 28.603 | 151.289 | 1.00 | 24.22 | B | C |
| ATOM | 1637 | GB | PHE | B | 153 | 57.010 | 28.041 | 150.147 | 1.00 | 21.10 | B | C |
| ATOM | 1638 | CG | PHE | B | 153 | 55.531 | 28.101 | 150.419 | 1.00 | 21.18 | B | C |
| ATOM | 1639 | CD1 | PHE | B | 153 | 54.916 | 27.141 | 151.229 | 1.00 | 19.72 | B | C |
| ATOM | 1640 | CD2 | PHE | B | 153 | 54.761 | 29.150 | 149.924 | 1.00 | 18.40 | B | C |
| ATOM | 1641 | CE1 | PHE | B | 153 | 53.560 | 27.231 | 151.541 | 1.00 | 18.13 | B | C |
| ATOM | 1642 | CE2 | PHE | B | 153 | 53.404 | 29.249 | 150.231 | 1.00 | 15.81 | B | C |
| ATOM | 1643 | CZ | PHE | B | 153 | 52.803 | 28.291 | 151.040 | 1.00 | 17.61 | B | C |
| ATOM | 1644 | C | PHE | B | 153 | 59.338 | 28.362 | 151.045 | 1.00 | 23.36 | B | C |
| ATOM | 1645 | O | PHE | B | 153 | 59.910 | 28.853 | 150.079 | 1.00 | 25.84 | B | O |
| ATOM | 1646 | N | THR | B | 154 | 59.951 | 27.596 | 151.938 | 1.00 | 24.03 | B | N |
| ATOM | 1647 | CA | THR | B | 154 | 61.375 | 27.302 | 151.858 | 1.00 | 22.52 | B | C |
| ATOM | 1648 | CB | THR | B | 154 | 62.075 | 27.739 | 153.149 | 1.00 | 21.34 | B | C |
| ATOM | 1649 | OG1 | THR | B | 154 | 61.867 | 29.138 | 153.328 | 1.00 | 25.73 | B | O |
| ATOM | 1650 | CG2 | THR | B | 154 | 63.567 | 27.461 | 153.091 | 1.00 | 20.97 | B | C |
| ATOM | 1651 | C | THR | B | 154 | 61.629 | 25.824 | 151.638 | 1.00 | 21.11 | B | C |
| ATOM | 1652 | O | THR | B | 154 | 61.050 | 24.977 | 152.317 | 1.00 | 21.31 | B | O |
| ATOM | 1653 | N | ARG | B | 155 | 62.502 | 25.521 | 150.688 | 1.00 | 21.15 | B | N |
| ATOM | 1654 | CA | ARG | B | 155 | 62.831 | 24.138 | 150.385 | 1.00 | 21.88 | B | C |
| ATOM | 1655 | CB | ARG | B | 155 | 63.248 | 23.996 | 148.919 | 1.00 | 23.11 | B | C |
| ATOM | 1656 | CC | ARG | B | 155 | 63.532 | 22.559 | 148.524 | 1.00 | 20.37 | B | C |
| ATOM | 1657 | CD | ARG | B | 155 | 64.052 | 22.459 | 147.121 | 1.00 | 21.66 | B | C |
| ATOM | 1658 | NE | ARG | B | 155 | 64.381 | 21.083 | 146.772 | 1.00 | 18.52 | B | N |
| ATOM | 1659 | CZ | ARG | B | 155 | 64.905 | 20.721 | 145.610 | 1.00 | 17.45 | B | C |
| ATOM | 1660 | NH1 | ARC | B | 155 | 65.158 | 21.637 | 144.689 | 1.00 | 16.66 | B | N |
| ATOM | 1661 | NH2 | ARG | B | 155 | 65.174 | 19.447 | 145.372 | 1.00 | 15.63 | B | N |
| ATOM | 1662 | C | ARG | B | 155 | 63.963 | 23.657 | 151.284 | 1.00 | 22.35 | B | C |
| ATOM | 1663 | O | ARC | B | 155 | 64.973 | 24.341 | 151.435 | 1.00 | 23.6S | B | O |
| ATOM | 1664 | N | VAL | B | 156 | 63.784 | 22.486 | 151.888 | 1.00 | 21.22 | B | N |
| ATOM | 1665 | CA | VAL | B | 156 | 64.795 | 21.904 | 152.760 | 1.00 | 21.38 | B | C |
| ATOM | 1666 | CB | VAL | B | 156 | 64.384 | 22.006 | 154.252 | 1.00 | 21.61 | B | C |
| ATOM | 1667 | CO1 | VAL | B | 156 | 64.096 | 23.462 | 154.600 | 1.00 | 19.88 | B | C |
| ATOM | 1668 | CO2 | VAL | B | 156 | 63.158 | 21.135 | 154.537 | 1.00 | 20.58 | B | C |
| ATOM | 1669 | C | VAL | B | 156 | 64.951 | 20.447 | 152.345 | 1.00 | 23.15 | B | C |
| ATOM | 1670 | O | VAL | B | 156 | 64.240 | 19.988 | 151.456 | 1.00 | 23.36 | B | O |
| ATOM | 1671 | N | TYR | B | 157 | 65.864 | 19.717 | 152.982 | 1.00 | 24.51 | B | N |
| ATOM | 1672 | CA | TYR | B | 157 | 66.100 | 18.321 | 152.613 | 1.00 | 25.70 | B | C |
| ATOM | 1673 | CB | TYR | B | 157 | 67.475 | 18.190 | 151.956 | 1.00 | 23.43 | B | C |
| ATOM | 1674 | CG | TYR | B | 157 | 67.634 | 19.050 | 150.728 | 1.00 | 23.94 | B | C |
| ATOM | 1675 | CD1 | TYR | B | 157 | 67.800 | 20.430 | 150.837 | 1.00 | 22.81 | B | C |
| ATOM | 1676 | CE1 | TYR | B | 157 | 67.891 | 21.233 | 149.703 | 1.00 | 24.82 | B | C |
| ATOM | 1677 | CD2 | TYR | B | 157 | 67.567 | 18.492 | 149.451 | 1.00 | 24.09 | B | C |
| ATOM | 1678 | CE2 | TYR | B | 157 | 67.658 | 19.285 | 148.313 | 1.00 | 24.05 | B | C |
| ATOM | 1679 | CZ | TYR | B | 157 | 67.820 | 20.652 | 148.445 | 1.00 | 25.74 | B | C |
| ATOM | 1680 | OH | TYR | E | 157 | 67.919 | 21.438 | 147.319 | 1.00 | 32.12 | B | O |
| ATOM | 1681 | C | TYR | B | 157 | 65.991 | 17.297 | 153.735 | 1.00 | 27.77 | B | C |
| ATOM | 1682 | O | TYR | B | 157 | 66.644 | 16.254 | 153.696 | 1.00 | 28.36 | B | O |
| ATOM | 1683 | N | SER | B | 158 | 65.162 | 17.576 | 154.731 | 1.00 | 29.96 | B | N |
| ATOM | 1684 | CA | SER | B | 158 | 65.006 | 16.641 | 155.834 | 1.00 | 32.14 | B | C |
| ATOM | 1685 | CB | SER | B | 158 | 65.751 | 17.144 | 157.077 | 1.00 | 32.84 | B | C |
| ATOM | 1686 | OG | SER | B | 158 | 65.274 | 18.414 | 157.490 | 1.00 | 34.79 | B | O |
| ATOM | 1687 | C | SER | B | 158 | 63.549 | 16.412 | 156.178 | 1.00 | 33.22 | B | C |
| ATOM | 1688 | O | SER | B | 158 | 62.667 | 17.155 | 155.750 | 1.00 | 29.83 | B | O |
| ATOM | 1689 | N | ARG | B | 159 | 63.319 | 15.376 | 156.973 | 1.00 | 37.03 | B | N |
| ATOM | 1690 | CA | ARO | B | 159 | 61.990 | 14.999 | 157.418 | 1.00 | 40.29 | B | C |
| ATOM | 1691 | CB | ARG | B | 159 | 62.071 | 13.676 | 158.176 | 1.00 | 46.56 | B | C |
| ATOM | 1692 | CG | ARG | E | 159 | 60.957 | 12.699 | 157.863 | 1.00 | 57.91 | B | C |
| ATOM | 1693 | CD | ARG | B | 159 | 61.049 | 11.467 | 158.757 | 1.00 | 64.70 | B | C |
| ATOM | 1694 | NE | ARG | B | 159 | 59.974 | 10.514 | 158.490 | 1.00 | 70.47 | B | N |
| ATOM | 1695 | CZ | ARG | B | 159 | 59.694 | 9.466 | 159.260 | 1.00 | 73.51 | B | C |
| ATOM | 1696 | NE1 | ARG | B | 159 | 58.698 | 8.651 | 158.937 | 1.00 | 74.22 | B | N |
| ATOM | 1697 | NH2 | ARG | B | 159 | 60.406 | 9.237 | 160.357 | 1.00 | 74.50 | B | N |
| ATOM | 1698 | C | ARG | B | 159 | 61.395 | 16.078 | 158.325 | 1.00 | 38.61 | B | C |
| ATOM | 1699 | O | ARG | B | 159 | 60.264 | 15.949 | 158.781 | 1.00 | 39.20 | B | O |
| ATOM | 1700 | N | ASP | B | 160 | 62.160 | 17.135 | 158.583 | 1.00 | 37.90 | B | N |
| ATOM | 1701 | CA | ASP | B | 160 | 61.707 | 18.233 | 159.438 | 1.00 | 36.97 | B | C |
| ATOM | 1702 | CB | ASP | B | 160 | 62.894 | 19.043 | 159.971 | 1.00 | 42.42 | B | C |
| ATOM | 1703 | CG | ASP | B | 160 | 63.908 | 18.195 | 160.698 | 1.00 | 46.87 | B | C |
| ATOM | 1704 | OD1 | ASP | B | 160 | 63.505 | 17.346 | 161.523 | 1.00 | 49.44 | B | O |
| ATOM | 1705 | OD2 | ASP | B | 160 | 65.116 | 18.393 | 160.451 | 1.00 | 50.63 | B | O |
| ATOM | 1706 | C | ASP | B | 160 | 60.784 | 19.198 | 158.706 | 1.00 | 33.52 | B | C |
| ATOM | 1707 | O | ASP | B | 160 | 60.165 | 20.065 | 159.326 | 1.00 | 33.69 | B | O |
| ATOM | 1708 | N | ALA | B | 161 | 60.706 | 19.065 | 157.389 | 1.00 | 29.73 | B | N |
| ATOM | 1709 | CA | ALA | B | 161 | 59.864 | 19.948 | 156.594 | 1.00 | 25.02 | B | C |
| ATOM | 1710 | CB | ALA | B | 161 | 60.019 | 19.620 | 155.116 | 1.00 | 24.48 | B | C |
| ATOM | 1711 | C | ALA | B | 161 | 58.408 | 19.808 | 157.005 | 1.00 | 22.73 | B | C |
| ATOM | 1712 | O | ALA | B | 161 | 57.983 | 18.743 | 157.453 | 1.00 | 18.59 | B | O |
| ATOM | 1713 | N | ASP | B | 162 | 57.644 | 20.886 | 156.865 | 1.00 | 20.01 | B | N |
| ATOM | 1714 | CA | ASP | B | 162 | 56.226 | 20.835 | 157.207 | 1.00 | 19.49 | B | C |
| ATOM | 1715 | CB | ASP | B | 162 | 55.625 | 22.240 | 157.298 | 1.00 | 17.17 | B | C |
| ATOM | 1716 | CG | ASP | B | 162 | 56.279 | 23.087 | 158.365 | 1.00 | 15.31 | B | C |
| ATOM | 1717 | OD1 | ASP | B | 162 | 56.422 | 22.601 | 159.505 | 1.00 | 16.45 | B | O |
| ATOM | 1718 | OD2 | ASP | B | 162 | 56.643 | 24.242 | 158.066 | 1.00 | 14.81 | B | O |
| ATOM | 1719 | C | ASP | B | 162 | 55.486 | 20.055 | 156.128 | 1.00 | 20.86 | B | C |
| ATOM | 1720 | O | ASP | B | 162 | 54.620 | 19.240 | 156.417 | 1.00 | 19.12 | B | O |
| ATOM | 1721 | N | ILE | B | 163 | 55.836 | 20.312 | 154.875 | 1.00 | 20.30 | B | N |
| ATOM | 1722 | CA | ILE | B | 163 | 55.183 | 19.646 | 153.760 | 1.00 | 17.16 | B | C |
| ATOM | 1723 | CB | ILE | B | 163 | 54.640 | 20.692 | 152.748 | 1.00 | 19.96 | B | C |
| ATOM | 1724 | CO2 | ILE | B | 163 | 54.032 | 19.995 | 151.530 | 1.00 | 16.75 | B | C |
| ATOM | 1725 | CO1 | ILE | B | 163 | 53.611 | 21.587 | 153.449 | 1.00 | 20.77 | B | C |
| ATOM | 1726 | OD1 | ILE | B | 163 | 53.139 | 22.769 | 152.625 | 1.00 | 16.61 | B | C |
| ATOM | 1727 | C | ILE | B | 163 | 56.138 | 18.694 | 153.058 | 1.00 | 15.84 | B | C |
| ATOM | 1728 | O | ILE | B | 163 | 57.096 | 19.114 | 152.411 | 1.00 | 16.45 | B | O |
| ATOM | 1729 | N | VAL | B | 164 | 55.883 | 17.405 | 153.207 | 1.00 | 13.42 | B | N |
| ATOM | 1730 | CA | VAL | B | 164 | 56.711 | 16.408 | 152.567 | 1.00 | 13.63 | B | C |
| ATOM | 1731 | OB | VAL | B | 164 | 56.977 | 15.221 | 153.498 | 1.00 | 13.46 | B | C |
| ATOM | 1732 | CG1 | VAL | B | 164 | 57.737 | 14.136 | 152.750 | 1.00 | 11.25 | B | C |
| ATOM | 1733 | CG2 | VAL | B | 164 | 57.773 | 15.694 | 154.713 | 1.00 | 12.79 | B | C |
| ATOM | 1734 | C | VAL | B | 164 | 55.991 | 15.939 | 151.310 | 1.00 | 14.94 | B | C |
| ATOM | 1735 | O | VAL | B | 164 | 54.826 | 15.535 | 151.358 | 1.00 | 15.76 | B | O |
| ATOM | 1736 | N | ILE | B | 165 | 56.698 | 16.015 | 150.187 | 1.00 | 13.79 | B | N |
| ATOM | 1737 | CA | ILE | B | 165 | 56.165 | 15.636 | 148.886 | 1.00 | 12.71 | B | C |
| ATOM | 1738 | OB | ILE | B | 165 | 56.472 | 16.714 | 147.857 | 1.00 | 14.97 | B | C |
| ATOM | 1739 | CO2 | ILE | B | 165 | 55.912 | 16.306 | 146.503 | 1.00 | 13.61 | B | C |
| ATOM | 1740 | CO1 | ILE | B | 165 | 55.919 | 18.059 | 148.348 | 1.00 | 13.19 | B | C |
| ATOM | 1741 | CD1 | LLE | B | 165 | 56.377 | 19.259 | 147.543 | 1.00 | 14.12 | B | C |
| ATOM | 1742 | C | ILE | B | 165 | 56.749 | 14.334 | 148.381 | 1.00 | 13.67 | B | C |
| ATOM | 1743 | O | ILE | B | 165 | 57.949 | 14.096 | 148.491 | 1.00 | 11.91 | B | O |
| ATOM | 1744 | N | GLN | B | 166 | 55.903 | 13.481 | 147.820 | 1.00 | 14.64 | B | N |
| ATOM | 1745 | CA | GLN | B | 166 | 56.400 | 12.230 | 147.291 | 1.00 | 17.38 | B | C |
| ATOM | 1746 | CB | GLN | B | 166 | 56.599 | 11.209 | 148.412 | 1.00 | 20.52 | B | C |
| ATOM | 1747 | CG | GLN | B | 166 | 55.328 | 10.614 | 148.956 | 1.00 | 28.99 | B | C |
| ATOM | 1748 | CD | GLN | B | 166 | 55.589 | 9.395 | 149.828 | 1.00 | 34.11 | B | C |
| ATOM | 1749 | OB1 | GLN | B | 166 | 55.972 | 8.329 | 149.334 | 1.00 | 34.45 | B | O |
| ATOM | 1750 | NB2 | GLN | B | 166 | 55.392 | 9.550 | 151.134 | 1.00 | 36.00 | B | N |
| ATOM | 1751 | C | GLN | B | 166 | 55.511 | 11.625 | 146.226 | 1.00 | 16.18 | B | C |
| ATOM | 1752 | O | QLN | B | 166 | 54.304 | 11.856 | 146.204 | 1.00 | 14.81 | B | O |
| ATOM | 1753 | N | PHE | B | 167 | 56.141 | 10.859 | 145.339 | 1.00 | 14.40 | B | N |
| ATOM | 1754 | CA | PHE | B | 167 | 55.463 | 10.147 | 144.274 | 1.00 | 13.46 | B | C |
| ATOM | 1755 | CB | PHE | B | 167 | 56.237 | 10.257 | 142.959 | 1.00 | 9.79 | B | C |
| ATOM | 1756 | CG | PHE | B | 167 | 56.265 | 11.642 | 142.386 | 1.00 | 11.55 | B | C |
| ATOM | 1757 | CD1 | PHE | B | 167 | 57.172 | 12.581 | 142.852 | 1.00 | 12.24 | B | C |
| ATOM | 1758 | CD2 | PHE | B | 167 | 55.371 | 12.012 | 141.388 | 1.00 | 12.04 | B | C |
| ATOM | 1759 | CE1 | PEE | B | 167 | 57.191 | 13.874 | 142.334 | 1.00 | 12.05 | B | C |
| ATOM | 1760 | CE2 | PHE | B | 167 | 55.379 | 13.306 | 140.865 | 1.00 | 11.98 | B | C |
| ATOM | 1761 | CZ | PHE | B | 167 | 56.288 | 14.237 | 141.338 | 1.00 | 13.47 | B | C |
| ATOM | 1762 | C | PHE | B | 167 | 55.414 | 8.685 | 144.681 | 1.00 | 14.78 | B | C |
| ATOM | 1763 | O | PHE | B | 167 | 56.389 | 8.160 | 145.202 | 1.00 | 16.19 | B | O |
| ATOM | 1764 | N | GLY | B | 168 | 54.283 | 8.028 | 144.446 | 1.00 | 16.63 | B | N |
| ATOM | 1765 | CA | GLY | B | 168 | 54.170 | 6.626 | 144.795 | 1.00 | 18.19 | B | C |
| ATOM | 1766 | C | GLY | B | 168 | 53.143 | 5.900 | 143.951 | 1.00 | 20.06 | B | C |
| ATOM | 1767 | O | GLY | B | 168 | 52.352 | 6.535 | 143.260 | 1.00 | 21.45 | B | O |
| ATOM | 1768 | N | VAL | B | 169 | 53.168 | 4.572 | 143.988 | 1.00 | 21.07 | B | N |
| ATOM | 1769 | CA | VAL | B | 169 | 52.206 | 3.764 | 143.247 | 1.00 | 22.74 | B | C |
| ATOM | 1770 | CB | VAL | B | 169 | 52.875 | 2.967 | 142.109 | 1.00 | 22.64 | B | C |
| ATOM | 1771 | GG1 | VAL | B | 169 | 53.593 | 3.913 | 141.170 | 1.00 | 22.95 | B | C |
| ATOM | 1772 | CG2 | VAL | B | 169 | 53.832 | 1.943 | 142.686 | 1.00 | 23.18 | B | C |
| ATOM | 1773 | C | VAL | B | 169 | 51.547 | 2.773 | 144.203 | 1.00 | 24.58 | B | C |
| ATOM | 1774 | O | VAL | B | 169 | 52.210 | 2.174 | 145.055 | 1.00 | 24.53 | B | O |
| ATOM | 1775 | N | ALA | B | 170 | 50.238 | 2.607 | 144.061 | 1.00 | 25.72 | B | N |
| ATOM | 1776 | CA | ALA | B | 170 | 49.498 | 1.690 | 144.912 | 1.00 | 25.50 | B | C |
| ATOM | 1777 | CB | ALA | B | 170 | 49.845 | 0.256 | 144.543 | 1.00 | 24.40 | B | C |
| ATOM | 1778 | C | ALA | B | 170 | 49.826 | 1.959 | 146.379 | 1.00 | 25.00 | B | C |
| ATOM | 1779 | O | ALA | B | 170 | 49.761 | 3.106 | 146.836 | 1.00 | 23.10 | B | O |
| ATOM | 1780 | N | GLU | B | 171 | 50.179 | 0.902 | 147.105 | 1.00 | 26.18 | B | N |
| ATOM | 1781 | CA | GLU | B | 171 | 50.521 | 1.002 | 148.523 | 1.00 | 30.44 | B | C |
| ATOM | 1782 | CB | GLU | B | 171 | 50.756 | −0.391 | 149.113 | 1.00 | 37.69 | B | C |
| ATOM | 1783 | CG | GLU | B | 171 | 49.929 | −1.502 | 148.484 | 1.00 | 50.32 | B | C |
| ATOM | 1784 | CD | GLU | B | 171 | 48.452 | −1.388 | 148.800 | 1.00 | 56.96 | B | C |
| ATOM | 1785 | OE1 | GLU | B | 171 | 47.671 | −2.226 | 148.296 | 1.00 | 59.68 | B | O |
| ATOM | 1786 | OE2 | GLU | B | 171 | 48.074 | −0.464 | 149.555 | 1.00 | 61.18 | B | O |
| ATOM | 1787 | C | GLU | B | 171 | 51.810 | 1.802 | 148.647 | 1.00 | 28.21 | B | C |
| ATOM | 1788 | O | GLU | B | 171 | 52.839 | 1.399 | 148.108 | 1.00 | 26.23 | B | O |
| ATOM | 1789 | N | HIS | B | 172 | 51.776 | 2.922 | 149.360 | 1.00 | 26.31 | B | N |
| ATOM | 1790 | CA | HIS | B | 172 | 52.989 | 3.723 | 149.488 | 1.00 | 26.29 | B | C |
| ATOM | 1791 | CB | HIS | B | 172 | 52.935 | 4.898 | 148.500 | 1.00 | 21.05 | B | C |
| ATOM | 1792 | CG | HIS | B | 172 | 51.733 | 5.777 | 148.671 | 1.00 | 19.27 | B | C |
| ATOM | 1793 | CD2 | HIS | B | 172 | 51.536 | 6.874 | 149.438 | 1.00 | 17.23 | B | C |
| ATOM | 1794 | ND1 | HIS | B | 172 | 50.526 | 5.519 | 148.056 | 1.00 | 15.56 | B | N |
| ATOM | 1795 | CR1 | HiS | B | 172 | 49.638 | 6.416 | 148.441 | 1.00 | 14.96 | B | C |
| ATOM | 1796 | NE2 | HIS | B | 172 | 50.225 | 7.248 | 149.281 | 1.00 | 13.17 | B | N |
| ATOM | 1797 | C | HIS | B | 172 | 53.334 | 4.246 | 150.890 | 1.00 | 27.55 | B | C |
| ATOM | 1798 | O | HIS | B | 172 | 54.057 | 5.231 | 151.019 | 1.00 | 26.45 | B | O |
| ATOM | 1799 | N | GLY | B | 173 | 52.831 | 3.601 | 151.940 | 1.00 | 30.22 | B | N |
| ATOM | 1800 | CA | GLY | B | 173 | 53.154 | 4.070 | 153.280 | 1.00 | 32.63 | B | C |
| ATOM | 1801 | C | GLY | B | 173 | 51.972 | 4.307 | 154.198 | 1.00 | 35.23 | B | C |
| ATOM | 1802 | O | GLY | B | 173 | 51.827 | 3.621 | 155.209 | 1.00 | 39.12 | B | O |
| ATOM | 1803 | N | ASP | B | 174 | 51.138 | 5.289 | 153.875 | 1.00 | 33.34 | B | N |
| ATOM | 1804 | CA | ASP | B | 174 | 49.968 | 5.563 | 154.692 | 1.00 | 33.01 | B | C |
| ATOM | 1805 | CB | ASP | B | 174 | 49.343 | 6.899 | 154.299 | 1.00 | 34.56 | B | C |
| ATOM | 1806 | CG | ASP | B | 174 | 49.366 | 7.138 | 152.797 | 1.00 | 33.72 | B | C |
| ATOM | 1807 | OD1 | ASP | B | 174 | 49.119 | 6.191 | 152.023 | 1.00 | 30.99 | B | O |
| ATOM | 1808 | OD2 | ASP | B | 174 | 49.623 | 8.286 | 152.395 | 1.00 | 32.06 | B | O |
| ATOM | 1809 | C | ASP | B | 174 | 48.969 | 4.438 | 154.475 | 1.00 | 33.84 | B | C |
| ATOM | 1810 | O | ASP | B | 174 | 49.323 | 3.368 | 153.982 | 1.00 | 35.39 | B | O |
| ATOM | 1811 | N | GLY | B | 175 | 47.719 | 4.666 | 154.837 | 1.00 | 34.25 | B | N |
| ATOM | 1812 | CA | GLY | B | 175 | 46.732 | 3.620 | 154.641 | 1.00 | 36.75 | B | C |
| ATOM | 1813 | C | GLY | B | 175 | 45.836 | 3.952 | 153.474 | 1.00 | 35.04 | B | C |
| ATOM | 1814 | O | GLY | B | 175 | 44.705 | 3.478 | 153.390 | 1.00 | 37.61 | B | O |
| ATOM | 1815 | N | TYR | B | 176 | 46.359 | 4.768 | 152.566 | 1.00 | 33.73 | B | N |
| ATOM | 1816 | CA | TYR | B | 176 | 45.597 | 5.200 | 151.409 | 1.00 | 32.53 | B | C |
| ATOM | 1817 | CB | TYR | B | 176 | 45.285 | 6.690 | 151.545 | 1.00 | 35.60 | B | C |
| ATOM | 1818 | CO | TYR | B | 176 | 44.636 | 7.044 | 152.867 | 1.00 | 40.89 | B | C |
| ATOM | 1819 | CD1 | TYR | B | 176 | 45.334 | 6.907 | 154.070 | 1.00 | 43.33 | B | C |
| ATOM | 1820 | CE1 | TYR | B | 176 | 44.736 | 7.204 | 155.290 | 1.00 | 43.67 | B | C |
| ATOM | 1821 | CD2 | TYR | B | 176 | 43.318 | 7.492 | 152.922 | 1.00 | 42.09 | B | C |
| ATOM | 1822 | CE2 | TYR | B | 176 | 42.711 | 7.794 | 154.140 | 1.00 | 42.63 | B | C |
| ATOM | 1823 | CZ | TYR | B | 176 | 43.426 | 7.647 | 155.317 | 1.00 | 43.63 | B | C |
| ATOM | 1824 | OH | TYR | B | 176 | 42.835 | 7.947 | 156.519 | 1.00 | 45.29 | B | O |
| ATOM | 1825 | C | TYR | B | 176 | 46.309 | 4.931 | 150.089 | 1.00 | 30.01 | B | C |
| ATOM | 1826 | O | TYR | B | 176 | 46.907 | 5.825 | 149.303 | 1.00 | 29.44 | B | O |
| ATOM | 1827 | N | PRO | B | 177 | 46.255 | 3.686 | 149.604 | 1.00 | 29.94 | B | N |
| ATOM | 1828 | CD | PRO | B | 177 | 45.706 | 2.475 | 150.238 | 1.00 | 28.60 | B | C |
| ATOM | 1829 | CA | PRO | B | 177 | 46.918 | 3.366 | 148.335 | 1.00 | 28.82 | B | C |
| ATOM | 1830 | CB | PRO | B | 177 | 46.838 | 1.843 | 148.280 | 1.00 | 29.21 | B | C |
| ATOM | 1831 | CG | PRO | B | 177 | 45.590 | 1.533 | 149.067 | 1.00 | 30.96 | B | C |
| ATOM | 1832 | C | PRO | B | 177 | 46.253 | 4.029 | 147.128 | 1.00 | 28.62 | B | C |
| ATOM | 1833 | O | PRO | B | 177 | 45.070 | 4.372 | 147.167 | 1.00 | 30.61 | B | O |
| ATOM | 1834 | N | PHE | B | 178 | 47.023 | 4.225 | 146.062 | 1.00 | 26.71 | B | N |
| ATOM | 1835 | CA | PHE | B | 178 | 46.475 | 4.825 | 144.851 | 1.00 | 26.70 | B | C |
| ATOM | 1836 | CB | PHE | B | 178 | 47.568 | 5.547 | 144.055 | 1.00 | 22.42 | B | C |
| ATOM | 1837 | CG | PHE | B | 178 | 48.019 | 6.842 | 144.687 | 1.00 | 17.95 | B | C |
| ATOM | 1838 | CD1 | PHE | B | 178 | 47.108 | 7.871 | 144.919 | 1.00 | 16.47 | B | C |
| ATOM | 1839 | CD2 | PHE | B | 178 | 49.349 | 7.029 | 145.058 | 1.00 | 17.30 | B | C |
| ATOM | 1840 | CE1 | PHE | B | 178 | 47.511 | 9.069 | 145.511 | 1.00 | 14.31 | B | C |
| ATOM | 1841 | CE2 | PHE | B | 178 | 49.765 | 8.221 | 145.650 | 1.00 | 14.81 | B | C |
| ATOM | 1842 | CZ | PHE | B | 178 | 48.842 | 9.245 | 145.877 | 1.00 | 16.31 | B | C |
| ATOM | 1843 | C | PHE | B | 178 | 45.810 | 3.733 | 144.017 | 1.00 | 26.77 | B | C |
| ATOM | 1844 | O | PHE | B | 178 | 46.020 | 2.546 | 144.261 | 1.00 | 23.82 | B | O |
| ATOM | 1845 | N | ASP | B | 179 | 45.017 | 4.142 | 143.032 | 1.00 | 28.61 | B | N |
| ATOM | 1846 | CA | ASP | B | 179 | 44.259 | 3.209 | 142.203 | 1.00 | 29.98 | B | C |
| ATOM | 1847 | CB | ASP | B | 179 | 42.808 | 3.674 | 142.164 | 1.00 | 29.97 | B | C |
| ATOM | 1848 | CG | ASP | B | 179 | 42.698 | 5.131 | 141.810 | 1.00 | 30.41 | B | C |
| ATOM | 1849 | OD1 | ASP | B | 179 | 43.709 | 5.661 | 141.331 | 1.00 | 35.31 | B | O |
| ATOM | 1850 | OD2 | ASP | B | 179 | 41.631 | 5.750 | 141.995 | 1.00 | 33.11 | B | O |
| ATOM | 1851 | C | ASP | B | 179 | 44.731 | 2.976 | 140.768 | 1.00 | 30.43 | B | C |
| ATOM | 1852 | O | ASP | B | 179 | 43.924 | 2.646 | 139.909 | 1.00 | 30.49 | B | O |
| ATOM | 1853 | N | GLY | B | 180 | 46.020 | 3.128 | 140.499 | 1.00 | 30.89 | B | N |
| ATOM | 1854 | CA | GLY | B | 180 | 46.493 | 2.905 | 139.146 | 1.00 | 29.05 | B | C |
| ATOM | 1855 | C | GLY | B | 180 | 46.142 | 4.064 | 138.237 | 1.00 | 29.44 | B | C |
| ATOM | 1856 | O | GLY | B | 180 | 45.789 | 5.131 | 138.723 | 1.00 | 25.58 | B | O |
| ATOM | 1857 | N | LYS | B | 181 | 46.217 | 3.849 | 136.925 | 1.00 | 29.06 | B | N |
| ATOM | 1858 | CA | LYS | B | 181 | 45.936 | 4.895 | 135.943 | 1.00 | 30.26 | B | C |
| ATOM | 1859 | CB | LYS | B | 181 | 46.100 | 4.322 | 134.535 | 1.00 | 33.27 | B | C |
| ATOM | 1860 | CG | LYS | B | 181 | 45.992 | 5.346 | 133.426 | 1.00 | 36.93 | B | C |
| ATOM | 1861 | CD | LYS | B | 181 | 46.258 | 4.704 | 132.077 | 1.00 | 41.28 | B | C |
| ATOM | 1862 | CE | LYS | B | 181 | 46.044 | 5.691 | 130.936 | 1.00 | 44.88 | B | C |
| ATOM | 1863 | NZ | LYS | B | 181 | 44.634 | 6.176 | 130.859 | 1.00 | 43.95 | B | N |
| ATOM | 1864 | C | LYS | B | 181 | 44.565 | 5.565 | 136.087 | 1.00 | 28.94 | B | C |
| ATOM | 1865 | O | LYS | B | 181 | 43.545 | 4.901 | 136.239 | 1.00 | 30.47 | B | O |
| ATOM | 1866 | N | ASP | B | 182 | 44.561 | 6.892 | 136.034 | 1.00 | 29.99 | B | N |
| ATOM | 1867 | CA | ASP | B | 182 | 43.347 | 7.692 | 136.166 | 1.00 | 30.47 | B | C |
| ATOM | 1868 | CB | ASP | B | 182 | 42.367 | 7.376 | 135.035 | 1.00 | 35.39 | B | C |
| ATOM | 1869 | CG | ASP | B | 182 | 42.877 | 7.826 | 133.684 | 1.00 | 38.40 | B | C |
| ATOM | 1870 | OD1 | ASP | B | 182 | 43.081 | 9.045 | 133.491 | 1.00 | 39.28 | B | O |
| ATOM | 1871 | OD2 | ASP | B | 182 | 43.078 | 6.954 | 132.814 | 1.00 | 42.49 | B | O |
| ATOM | 1872 | C | ASP | B | 182 | 42.642 | 7.509 | 137.506 | 1.00 | 31.65 | B | C |
| ATOM | 1873 | O | ASP | B | 182 | 43.099 | 6.765 | 138.363 | 1.00 | 32.44 | B | O |
| ATOM | 1874 | N | GLY | B | 183 | 41.509 | 8.181 | 137.670 | 1.00 | 29.41 | B | N |
| ATOM | 1875 | CA | GLY | B | 183 | 40.774 | 8.091 | 138.912 | 1.00 | 27.84 | B | C |
| ATOM | 1876 | C | GLY | B | 183 | 41.335 | 9.123 | 139.864 | 1.00 | 27.88 | B | C |
| ATOM | 1877 | O | GLY | B | 183 | 41.355 | 10.310 | 139.549 | 1.00 | 29.24 | B | O |
| ATOM | 1878 | N | LEU | B | 184 | 41.792 | 8.678 | 141.030 | 1.00 | 27.15 | B | N |
| ATOM | 1879 | CA | LEU | B | 184 | 42.376 | 9.585 | 142.015 | 1.00 | 25.82 | B | C |
| ATOM | 1880 | CB | LEU | B | 184 | 42.493 | 8.878 | 143.359 | 1.00 | 27.95 | B | C |
| ATOM | 1881 | CG | LEU | B | 184 | 42.940 | 9.772 | 144.507 | 1.00 | 30.41 | B | C |
| ATOM | 1882 | CD1 | LEU | B | 184 | 41.855 | 10.797 | 144.779 | 1.00 | 28.19 | B | C |
| ATOM | 1883 | CD2 | LEU | B | 184 | 43.205 | 8.927 | 145.746 | 1.00 | 34.06 | B | C |
| ATOM | 1884 | C | LEU | B | 184 | 43.771 | 9.971 | 141.520 | 1.00 | 22.60 | B | C |
| ATOM | 1885 | O | LEU | B | 184 | 44.593 | 9.106 | 141.265 | 1.00 | 17.85 | B | O |
| ATOM | 1886 | N | LEU | B | 185 | 44.047 | 11.262 | 141.397 | 1.00 | 22.16 | B | N |
| ATOM | 1887 | CA | LEU | B | 185 | 45.346 | 11.700 | 140.884 | 1.00 | 22.33 | B | C |
| ATOM | 1888 | CB | LEU | B | 185 | 45.177 | 13.002 | 140.095 | 1.00 | 19.27 | B | C |
| ATOM | 1889 | CG | LEU | B | 185 | 44.127 | 12.970 | 138.979 | 1.00 | 20.81 | B | C |
| ATOM | 1890 | CD1 | LEU | B | 185 | 44.110 | 14.305 | 138.252 | 1.00 | 12.81 | B | C |
| ATOM | 1891 | CD2 | LEU | B | 185 | 44.425 | 11.823 | 138.013 | 1.00 | 17.71 | B | C |
| ATOM | 1892 | C | LEU | B | 185 | 46.400 | 11.897 | 141.965 | 1.00 | 23.14 | B | C |
| ATOM | 1893 | O | LEU | B | 185 | 47.593 | 11.658 | 141.749 | 1.00 | 22.98 | B | O |
| ATOM | 1894 | N | ALA | B | 186 | 45.948 | 12.331 | 143.131 | 1.00 | 23.03 | B | N |
| ATOM | 1895 | CA | ALA | B | 186 | 46.830 | 12.585 | 144.255 | 1.00 | 22.63 | B | C |
| ATOM | 1896 | CB | ALA | B | 186 | 47.717 | 13.768 | 143.942 | 1.00 | 21.82 | B | C |
| ATOM | 1897 | C | ALA | B | 186 | 45.974 | 12.896 | 145.472 | 1.00 | 23.10 | B | C |
| ATOM | 1898 | O | ALA | B | 186 | 44.751 | 12.932 | 145.378 | 1.00 | 25.16 | B | O |
| ATOM | 1899 | N | HIS | B | 187 | 46.622 | 13.104 | 146.613 | 1.00 | 22.01 | B | N |
| ATOM | 1900 | CA | HIS | B | 187 | 45.927 | 13.458 | 147.840 | 1.00 | 21.61 | B | C |
| ATOM | 1901 | CB | HIS | B | 187 | 45.218 | 12.237 | 148.451 | 1.00 | 19.44 | B | C |
| ATOM | 1902 | CG | HIS | B | 187 | 46.126 | 11.110 | 148.838 | 1.00 | 21.62 | B | C |
| ATOM | 1903 | CD2 | HIS | B | 187 | 47.343 | 11.101 | 149.434 | 1.00 | 23.01 | B | C |
| ATOM | 1904 | ND1 | HIS | B | 187 | 45.771 | 9.787 | 148.673 | 1.00 | 20.29 | B | N |
| ATOM | 1905 | CE1 | HiS | B | 187 | 46.730 | 9.013 | 149.151 | 1.00 | 20.79 | B | C |
| ATOM | 1906 | NE2 | HIS | B | 187 | 47.695 | 9.786 | 149.619 | 1.00 | 19.38 | B | N |
| ATOM | 1907 | C | HIS | B | 187 | 46.898 | 14.097 | 148.822 | 1.00 | 22.17 | B | C |
| ATOM | 1908 | O | HIS | B | 187 | 48.086 | 13.786 | 148.818 | 1.00 | 21.16 | B | O |
| ATOM | 1909 | N | ALA | B | 188 | 46.386 | 15.015 | 149.637 | 1.00 | 19.90 | B | N |
| ATOM | 1910 | CA | ALA | B | 188 | 47.191 | 15.727 | 150.615 | 1.00 | 21.04 | B | C |
| ATOM | 1911 | CB | ALA | B | 188 | 47.386 | 17.165 | 150.169 | 1.00 | 16.20 | B | C |
| ATOM | 1912 | C | ALA | B | 188 | 46.511 | 15.689 | 151.981 | 1.00 | 24.29 | B | C |
| ATOM | 1913 | O | ALA | B | 188 | 45.342 | 15.313 | 152.091 | 1.00 | 23.00 | B | 0 |
| ATOM | 1914 | N | PHE | B | 189 | 47.248 | 16.076 | 153.020 | 1.00 | 25.22 | B | N |
| ATOM | 1915 | CA | PHE | B | 189 | 46.712 | 16.081 | 154.373 | 1.00 | 23.69 | B | C |
| ATOM | 1916 | CB | PHE | B | 189 | 47.532 | 15.162 | 155.290 | 1.00 | 23.93 | B | C |
| ATOM | 1917 | CG | PHE | B | 189 | 47.565 | 13.731 | 154.839 | 1.00 | 26.85 | B | C |
| ATOM | 1918 | CD1 | PUE | B | 189 | 48.483 | 13.308 | 153.881 | 1.00 | 29.60 | B | C |
| ATOM | 1919 | CD2 | PHE | B | 189 | 46.668 | 12.806 | 155.358 | 1.00 | 27.63 | B | C |
| ATOM | 1920 | CE1 | PHE | B | 189 | 48.510 | 11.984 | 153.447 | 1.00 | 27.95 | B | C |
| ATOM | 1921 | CE2 | PHE | B | 189 | 46.685 | 11.480 | 154.930 | 1.00 | 29.13 | B | C |
| ATOM | 1922 | CZ | PHE | B | 189 | 47.609 | 11.069 | 153.972 | 1.00 | 29.53 | B | C |
| ATOM | 1923 | C | PHE | B | 189 | 46.712 | 17.491 | 154.932 | 1.00 | 24.15 | B | C |
| ATOM | 1924 | O | PHE | B | 189 | 47.600 | 18.286 | 154.632 | 1.00 | 23.64 | B | O |
| ATOM | 1925 | N | PRO | B | 190 | 45.703 | 17.818 | 155.761 | 1.00 | 25.45 | B | N |
| ATOM | 1926 | CD | PRO | B | 190 | 44.626 | 16.901 | 156.165 | 1.00 | 24.82 | B | C |
| ATOM | 1927 | CA | PRO | B | 190 | 45.535 | 19.135 | 156.399 | 1.00 | 23.84 | B | C |
| ATOM | 1928 | CB | PRO | B | 190 | 44.211 | 18.998 | 157.151 | 1.00 | 23.85 | B | C |
| ATOM | 1929 | CG | PRO | B | 190 | 43.517 | 17.859 | 156.470 | 1.00 | 27.72 | B | C |
| ATOM | 1930 | C | PRO | B | 190 | 46.693 | 19.452 | 157.355 | 1.00 | 24.12 | B | C |
| ATOM | 1931 | O | PRO | B | 190 | 47.364 | 18.544 | 157.851 | 1.00 | 24.59 | B | O |
| ATOM | 1932 | N | PRO | B | 191 | 46.929 | 20.745 | 157.633 | 1.00 | 23.67 | B | N |
| ATOM | 1933 | CD | PRO | B | 191 | 46.130 | 21.889 | 157.160 | 1.00 | 22.01 | B | C |
| ATOM | 1934 | CA | PRO | B | 191 | 48.005 | 21.193 | 158.527 | 1.00 | 24.52 | B | C |
| ATOM | 1935 | CB | PRO | B | 191 | 47.668 | 22.660 | 158.756 | 1.00 | 23.21 | B | C |
| ATOM | 1936 | CG | PRO | B | 191 | 47.036 | 23.056 | 157.463 | 1.00 | 23.95 | B | C |
| ATOM | 1937 | C | PRO | B | 191 | 48.077 | 20.402 | 159.834 | 1.00 | 26.78 | B | C |
| ATOM | 1938 | O | PRO | B | 191 | 47.054 | 19.982 | 160.374 | 1.00 | 27.80 | B | O |
| ATOM | 1939 | N | GLY | B | 192 | 49.294 | 20.207 | 160.334 | 1.00 | 26.68 | B | N |
| ATOM | 1940 | CA | GLY | B | 192 | 49.490 | 19.459 | 161.563 | 1.00 | 25.28 | B | C |
| ATOM | 1941 | C | GLY | B | 192 | 50.851 | 18.786 | 161.573 | 1.00 | 26.25 | B | C |
| ATOM | 1942 | O | GLY | B | 192 | 51.653 | 19.026 | 160.671 | 1.00 | 27.65 | B | O |
| ATOM | 1943 | N | PRO | B | 193 | 51.145 | 17.935 | 162.574 | 1.00 | 26.70 | B | N |
| ATOM | 1944 | CD | PRO | B | 193 | 50.356 | 17.760 | 163.808 | 1.00 | 23.89 | B | C |
| ATOM | 1945 | CA | PRO | B | 193 | 52.428 | 17.233 | 162.685 | 1.00 | 26.43 | B | C |
| ATOM | 1946 | CB | PRO | B | 193 | 52.572 | 17.049 | 164.189 | 1.00 | 26.13 | B | C |
| ATOM | 1947 | CG | PRO | B | 193 | 51.173 | 16.742 | 164.588 | 1.00 | 24.53 | B | C |
| ATOM | 1948 | C | PRO | B | 193 | 52.463 | 15.901 | 161.947 | 1.00 | 25.56 | B | C |
| ATOM | 1949 | O | PRO | B | 193 | 51.423 | 15.356 | 161.592 | 1.00 | 28.95 | B | O |
| ATOM | 1950 | N | GLY | B | 194 | 53.665 | 15.379 | 161.721 | 1.00 | 24.12 | B | N |
| ATOM | 1951 | CA | GLY | B | 194 | 53.803 | 14.100 | 161.046 | 1.00 | 22.38 | B | C |
| ATOM | 1952 | C | GLY | B | 194 | 53.338 | 14.098 | 159.605 | 1.00 | 23.14 | B | C |
| ATOM | 1953 | O | GLY | B | 194 | 53.602 | 15.036 | 158.868 | 1.00 | 21.24 | B | O |
| ATOM | 1954 | N | ILE | B | 195 | 52.633 | 13.048 | 159.202 | 1.00 | 24.37 | B | N |
| ATOM | 1955 | CA | ILE | B | 195 | 52.147 | 12.941 | 157.832 | 1.00 | 26.26 | B | C |
| ATOM | 1956 | CB | ILE | B | 195 | 51.440 | 11.590 | 157.613 | 1.00 | 27.60 | B | C |
| ATOM | 1957 | CG2 | ILE | B | 195 | 50.131 | 11.561 | 158.386 | 1.00 | 27.08 | B | C |
| ATOM | 1958 | CG1 | ILE | B | 195 | 51.200 | 11.365 | 156.119 | 1.00 | 29.76 | B | C |
| ATOM | 1959 | CD1 | ILE | B | 195 | 50.620 | 10.012 | 155.789 | 1.00 | 31.30 | B | C |
| ATOM | 1960 | C | ILE | B | 195 | 51.194 | 14.085 | 157.479 | 1.00 | 26.36 | B | C |
| ATOM | 1961 | O | ILE | B | 195 | 50.921 | 14.343 | 156.309 | 1.00 | 27.06 | B | O |
| ATOM | 1962 | N | GLN | B | 196 | 50.704 | 14.781 | 158.498 | 1.00 | 26.62 | B | N |
| ATOM | 1963 | CA | GLN | B | 196 | 49.790 | 15.901 | 158.298 | 1.00 | 26.46 | B | C |
| ATOM | 1964 | CE | GLN | B | 196 | 49.359 | 16.457 | 159.654 | 1.00 | 29.46 | B | C |
| ATOM | 1965 | CG | GLN | B | 196 | 47.864 | 16.383 | 159.914 | 1.00 | 38.14 | B | C |
| ATOM | 1966 | CD | GLN | B | 196 | 47.323 | 14.967 | 159.860 | 1.00 | 41.94 | B | C |
| ATOM | 1967 | OE1 | GLN | B | 196 | 46.116 | 14.751 | 159.940 | 1.00 | 44.27 | B | O |
| ATOM | 1968 | NE2 | GLN | B | 196 | 48.215 | 13.993 | 159.727 | 1.00 | 46.11 | B | N |
| ATOM | 1969 | C | GLN | B | 196 | 50.459 | 16.998 | 157.482 | 1.00 | 23.79 | B | C |
| ATOM | 1970 | O | GLN | B | 196 | 51.576 | 17.367 | 157.768 | 1.00 | 25.59 | B | O |
| ATOM | 1971 | N | GLY | B | 197 | 49.774 | 17.524 | 156.472 | 1.00 | 23.38 | B | N |
| ATOM | 1972 | CA | GLY | B | 197 | 50.364 | 18.568 | 155.650 | 1.00 | 18.35 | B | C |
| ATOM | 1973 | C | GLY | B | 197 | 51.149 | 18.047 | 154.450 | 1.00 | 20.05 | B | C |
| ATOM | 1974 | O | GLY | B | 197 | 51.606 | 18.835 | 153.619 | 1.00 | 21.28 | B | O |
| ATOM | 1975 | N | ASP | B | 198 | 51.308 | 16.727 | 154.354 | 1.00 | 18.69 | B | N |
| ATOM | 1976 | CA | ASP | B | 198 | 52.049 | 16.115 | 153.253 | 1.00 | 18.67 | B | C |
| ATOM | 1977 | CB | ASP | B | 198 | 52.557 | 14.730 | 153.668 | 1.00 | 17.24 | B | C |
| ATOM | 1978 | CG | ASP | B | 198 | 53.613 | 14.794 | 154.764 | 1.00 | 19.44 | B | C |
| ATOM | 1979 | OD1 | ASP | B | 198 | 53.978 | 15.912 | 155.160 | 1.00 | 14.37 | B | O |
| ATOM | 1980 | OD2 | ASP | B | 198 | 54.080 | 13.730 | 155.220 | 1.00 | 16.31 | B | O |
| ATOM | 1981 | C | ASP | B | 198 | 51.203 | 15.996 | 151.974 | 1.00 | 20.96 | B | C |
| ATOM | 1982 | O | ASP | B | 198 | 49.971 | 16.042 | 152.029 | 1.00 | 21.22 | B | O |
| ATOM | 1983 | N | ALA | B | 199 | 51.872 | 15.836 | 150.831 | 1.00 | 18.26 | B | N |
| ATOM | 1984 | CA | ALA | B | 199 | 51.197 | 15.722 | 149.536 | 1.00 | 17.33 | B | C |
| ATOM | 1985 | CB | ALA | B | 199 | 51.326 | 17.027 | 148.771 | 1.00 | 16.82 | B | C |
| ATOM | 1986 | C | ALA | B | 199 | 51.803 | 14.586 | 148.728 | 1.00 | 18.72 | B | C |
| ATOM | 1987 | O | ALA | B | 199 | 53.004 | 14.569 | 148.483 | 1.00 | 20.07 | B | O |
| ATOM | 1988 | N | HIS | B | 200 | 50.968 | 13.639 | 148.314 | 1.00 | 17.89 | B | N |
| ATOM | 1989 | CA | HIS | B | 200 | 51.424 | 12.481 | 147.555 | 1.00 | 15.98 | B | C |
| ATOM | 1990 | CB | HIS | B | 200 | 51.092 | 11.215 | 148.334 | 1.00 | 17.39 | B | C |
| ATOM | 1991 | CG | HIS | B | 200 | 51.623 | 11.212 | 149.736 | 1.00 | 20.36 | B | C |
| ATOM | 1992 | CD2 | HIS | B | 200 | 52.599 | 11.944 | 150.325 | 1.00 | 17.40 | B | C |
| ATOM | 1993 | ND1 | HIS | B | 200 | 51.160 | 10.349 | 150.704 | 1.00 | 18.51 | B | N |
| ATOM | 1994 | CB1 | HIS | B | 200 | 51.827 | 10.546 | 151.826 | 1.00 | 17.19 | B | C |
| ATOM | 1995 | NE2 | HIS | B | 200 | 52.706 | 11.508 | 151.623 | 1.00 | 17.14 | B | N |
| ATOM | 1996 | C | HIS | B | 200 | 50.786 | 12.427 | 146.168 | 1.00 | 18.64 | B | C |
| ATOM | 1997 | O | HIS | B | 200 | 49.591 | 12.668 | 146.016 | 1.00 | 18.44 | B | O |
| ATOM | 1998 | N | PHE | B | 201 | 51.594 | 12.105 | 145.160 | 1.00 | 19.00 | B | N |
| ATOM | 1999 | CA | PHE | B | 201 | 51.128 | 12.046 | 143.776 | 1.00 | 15.37 | B | C |
| ATOM | 2000 | CB | PHE | B | 201 | 51.929 | 13.027 | 142.912 | 1.00 | 16.11 | B | C |
| ATOM | 2001 | CG | PHE | B | 201 | 51.907 | 14.444 | 143.421 | 1.00 | 14.27 | B | C |
| ATOM | 2002 | CD1 | PHE | B | 201 | 52.747 | 14.840 | 144.459 | 1.00 | 15.41 | B | C |
| ATOM | 2003 | CD2 | PHE | B | 201 | 51.017 | 15.374 | 142.887 | 1.00 | 13.45 | B | C |
| ATOM | 2004 | GE1 | PHE | B | 201 | 52.704 | 16.143 | 144.961 | 1.00 | 15.16 | B | C |
| ATOM | 2005 | CE2 | PHE | B | 201 | 50.962 | 16.685 | 143.380 | 1.00 | 14.52 | B | C |
| ATOM | 2006 | CZ | PHE | B | 201 | 51.806 | 17.071 | 144.418 | 1.00 | 14.98 | B | C |
| ATOM | 2007 | C | PHE | B | 201 | 51.227 | 10.650 | 143.185 | 1.00 | 15.36 | B | C |
| ATOM | 2008 | O | PHE | B | 201 | 52.257 | 9.993 | 143.293 | 1.00 | 15.05 | B | O |
| ATOM | 2009 | N | ASP | B | 202 | 50.144 | 10.204 | 142.555 | 1.00 | 16.99 | B | N |
| ATOM | 2010 | CA | ASP | B | 202 | 50.094 | 8.877 | 141.941 | 1.00 | 18.47 | B | C |
| ATOM | 2011 | CB | ASP | B | 202 | 48.660 | 8.502 | 141.583 | 1.00 | 16.97 | B | C |
| ATOM | 2012 | CG | ASP | B | 202 | 48.546 | 7.080 | 141.116 | 1.00 | 17.69 | B | C |
| ATOM | 2013 | OD1 | ASP | B | 202 | 49.567 | 6.496 | 140.725 | 1.00 | 24.22 | B | O |
| ATOM | 2014 | OD2 | ASP | B | 202 | 47.442 | 6.530 | 141.132 | 1.00 | 22.67 | B | O |
| ATOM | 2015 | C | ASP | B | 202 | 50.944 | 8.843 | 140.673 | 1.00 | 17.99 | B | C |
| ATOM | 2016 | O | ASP | B | 202 | 50.604 | 9.460 | 139.670 | 1.00 | 16.40 | B | O |
| ATOM | 2017 | N | ASP | B | 203 | 52.039 | 8.101 | 140.722 | 1.00 | 18.18 | B | N |
| ATOM | 2018 | CA | ASP | B | 203 | 52.943 | 8.020 | 139.593 | 1.00 | 19.10 | B | C |
| ATOM | 2019 | CB | ASP | B | 203 | 54.360 | 7.745 | 140.098 | 1.00 | 18.63 | B | C |
| ATOM | 2020 | CG | ASP | B | 203 | 55.424 | 8.234 | 139.147 | 1.00 | 21.55 | B | C |
| ATOM | 2021 | OD1 | ASP | B | 203 | 55.153 | 9.165 | 138.359 | 1.00 | 22.68 | B | O |
| ATOM | 2022 | OD2 | ASP | B | 203 | 56.546 | 7.700 | 139.204 | 1.00 | 24.38 | B | O |
| ATOM | 2023 | C | ASP | B | 203 | 52.522 | 6.977 | 138.560 | 1.00 | 22.02 | B | C |
| ATOM | 2024 | O | ASP | B | 203 | 53.328 | 6.553 | 137.739 | 1.00 | 22.79 | B | O |
| ATOM | 2025 | N | ASP | B | 204 | 51.274 | 6.529 | 138.627 | 1.00 | 21.73 | B | N |
| ATOM | 2026 | CA | ASP | B | 204 | 50.772 | 5.589 | 137.631 | 1.00 | 20.62 | B | C |
| ATOM | 2027 | CB | ASP | B | 204 | 49.729 | 4.635 | 138.212 | 1.00 | 19.80 | B | C |
| ATOM | 2028 | CC | ASP | B | 204 | 50.304 | 3.270 | 138.551 | 1.00 | 22.45 | B | C |
| ATOM | 2029 | OD1 | ASP | B | 204 | 51.334 | 2.884 | 137.963 | 1.00 | 20.45 | B | O |
| ATOM | 2030 | OD2 | ASP | B | 204 | 49.709 | 2.571 | 139.399 | 1.00 | 23.05 | B | O |
| ATOM | 2031 | C | ASP | B | 204 | 50.123 | 6.478 | 136.570 | 1.00 | 20.79 | B | C |
| ATOM | 2032 | O | ASP | B | 204 | 49.739 | 6.016 | 135.504 | 1.00 | 23.31 | B | O |
| ATOM | 2033 | N | GLU | B | 205 | 50.008 | 7.763 | 136.890 | 1.00 | 18.69 | B | N |
| ATOM | 2034 | CA | GLU | B | 205 | 49.441 | 8.755 | 135.987 | 1.00 | 20.13 | B | C |
| ATOM | 2035 | CB | GLU | B | 205 | 48.874 | 9.931 | 136.778 | 1.00 | 20.83 | B | C |
| ATOM | 2036 | CG | GLU | B | 205 | 47.935 | 9.526 | 137.894 | 1.00 | 29.96 | B | C |
| ATOM | 2037 | CD | GLU | B | 205 | 46.627 | 8.986 | 137.377 | 1.00 | 33.57 | B | C |
| ATOM | 2038 | OE1 | GLU | B | 205 | 46.617 | 8.360 | 136.293 | 1.00 | 36.59 | B | O |
| ATOM | 2039 | OE2 | GLU | B | 205 | 45.607 | 9.185 | 138.060 | 1.00 | 40.04 | B | O |
| ATOM | 2040 | C | GLU | B | 205 | 50.560 | 9.285 | 135.099 | 1.00 | 21.01 | B | C |
| ATOM | 2041 | O | GLU | B | 205 | 51.725 | 9.238 | 135.477 | 1.00 | 19.13 | B | O |
| ATOM | 2042 | N | LEU | B | 206 | 50.207 | 9.795 | 133.924 | 1.00 | 18.61 | B | N |
| ATOM | 2043 | CA | LEU | B | 206 | 51.204 | 10.366 | 133.026 | 1.00 | 17.96 | B | C |
| ATOM | 2044 | CB | LEU | B | 206 | 50.758 | 10.242 | 131.568 | 1.00 | 18.74 | B | C |
| ATOM | 2045 | CG | LEU | B | 206 | 51.617 | 10.985 | 130.532 | 1.00 | 19.98 | B | C |
| ATOM | 2046 | CD1 | LEU | B | 206 | 53.062 | 10.519 | 130.627 | 1.00 | 18.61 | B | C |
| ATOM | 2047 | CD2 | LEU | B | 206 | 51.064 | 10.740 | 129.131 | 1.00 | 17.02 | B | C |
| ATOM | 2048 | C | LEU | B | 206 | 51.312 | 11.837 | 133.399 | 1.00 | 18.42 | B | C |
| ATOM | 2049 | O | LEU | B | 206 | 50.379 | 12.603 | 133.160 | 1.00 | 18.49 | B | O |
| ATOM | 2050 | N | TRP | B | 207 | 52.434 | 12.230 | 133.998 | 1.00 | 17.94 | B | N |
| ATOM | 2051 | CA | TRP | B | 207 | 52.618 | 13.622 | 134.401 | 1.00 | 16.68 | B | C |
| ATOM | 2052 | CB | TRP | B | 207 | 53.454 | 13.723 | 135.694 | 1.00 | 15.77 | B | C |
| ATOM | 2053 | CO | TRP | B | 207 | 52.752 | 13.143 | 136.870 | 1.00 | 14.61 | B | C |
| ATOM | 2054 | CD2 | TRP | B | 207 | 51.546 | 13.636 | 137.469 | 1.00 | 14.73 | B | C |
| ATOM | 2055 | CE2 | TRP | B | 207 | 51.165 | 12.708 | 138.464 | 1.00 | 14.79 | B | C |
| ATOM | 2056 | CE3 | TRP | B | 207 | 50.744 | 14.769 | 137.255 | 1.00 | 16.70 | B | C |
| ATOM | 2057 | CD1 | TRP | B | 207 | 53.051 | 11.979 | 137.511 | 1.00 | 13.93 | B | C |
| ATOM | 2058 | NE1 | TRP | B | 207 | 52.101 | 11.708 | 138.467 | 1.00 | 15.76 | B | N |
| ATOM | 2059 | CZ2 | TRP | B | 207 | 50.012 | 12.876 | 139.248 | 1.00 | 13.38 | B | C |
| ATOM | 2060 | CZ3 | TRP | B | 207 | 49.591 | 14.935 | 138.034 | 1.00 | 13.30 | B | C |
| ATOM | 2061 | CH2 | TRP | B | 207 | 49.241 | 13.990 | 139.016 | 1.00 | 13.83 | B | C |
| ATOM | 2062 | C | TRY | B | 207 | 53.267 | 14.435 | 133.299 | 1.00 | 16.25 | B | C |
| ATOM | 2063 | O | TRP | B | 207 | 54.230 | 14.003 | 132.665 | 1.00 | 14.91 | B | O |
| ATOM | 2064 | N | SER | B | 208 | 52.731 | 15.630 | 133.092 | 1.00 | 16.82 | B | N |
| ATOM | 2065 | CA | SER | B | 208 | 53.218 | 16.521 | 132.061 | 1.00 | 15.97 | B | C |
| ATOM | 2066 | CB | SER | B | 208 | 52.497 | 16.187 | 130.747 | 1.00 | 16.80 | B | C |
| ATOM | 2067 | OG | SER | B | 208 | 53.111 | 16.799 | 129.634 | 1.00 | 12.64 | B | O |
| ATOM | 2068 | C | SER | B | 208 | 52.924 | 17.953 | 132.496 | 1.00 | 17.15 | B | C |
| ATOM | 2069 | O | SER | B | 208 | 52.677 | 18.207 | 133.676 | 1.00 | 20.26 | B | O |
| ATOM | 2070 | N | LEU | B | 209 | 52.954 | 18.881 | 131.543 | 1.00 | 15.88 | B | N |
| ATOM | 2071 | CA | LEU | B | 209 | 52.680 | 20.294 | 131.800 | 1.00 | 15.39 | B | C |
| ATOM | 2072 | CB | LEU | B | 209 | 53.972 | 21.049 | 132.105 | 1.00 | 12.60 | B | C |
| ATOM | 2073 | CG | LEU | B | 209 | 54.144 | 20.758 | 133.401 | 1.00 | 16.29 | B | C |
| ATOM | 2074 | CD1 | LEU | B | 209 | 56.029 | 21.560 | 133.386 | 1.00 | 12.54 | B | C |
| ATOM | 2075 | CD2 | LEU | B | 209 | 53.904 | 21.118 | 134.638 | 1.00 | 12.78 | B | C |
| ATOM | 2076 | C | LEU | B | 209 | 52.044 | 20.901 | 130.558 | 1.00 | 16.86 | B | C |
| ATOM | 2077 | O | LEU | B | 209 | 52.335 | 20.471 | 129.453 | 1.00 | 16.65 | B | O |
| ATOM | 2078 | N | GLY | B | 210 | 51.181 | 21.898 | 130.739 | 1.00 | 20.56 | B | N |
| ATOM | 2079 | CA | GLY | B | 210 | 50.545 | 22.557 | 129.602 | 1.00 | 20.23 | B | C |
| ATOM | 2080 | C | GLY | B | 210 | 49.475 | 21.767 | 128.864 | 1.00 | 21.25 | B | C |
| ATOM | 2081 | O | GLY | B | 210 | 48.885 | 20.835 | 129.409 | 1.00 | 21.72 | B | O |
| ATOM | 2082 | N | LYS | B | 211 | 49.207 | 22.158 | 127.621 | 1.00 | 20.99 | B | N |
| ATOM | 2083 | CA | LYS | B | 211 | 48.217 | 21.473 | 126.795 | l.00 | 21.58 | B | C |
| ATOM | 2084 | CB | LYS | B | 211 | 47.879 | 22.314 | 125.560 | 1.00 | 22.10 | B | C |
| ATOM | 2085 | CG | LYS | B | 211 | 47.094 | 23.564 | 125.878 | 1.00 | 24.12 | B | C |
| ATOM | 2086 | CD | LYS | B | 211 | 46.828 | 24.410 | 124.647 | 1.00 | 27.19 | B | C |
| ATOM | 2087 | CE | LYS | B | 211 | 48.099 | 25.058 | 124.149 | 1.00 | 30.98 | B | C |
| ATOM | 2088 | NZ | LYS | B | 211 | 47.812 | 26.127 | 123.147 | 1.00 | 36.83 | B | N |
| ATOM | 2089 | C | LYS | B | 211 | 48.804 | 20.142 | 126.355 | 1.00 | 22.13 | B | C |
| ATOM | 2090 | O | LYS | B | 211 | 50.029 | 19.997 | 126.286 | 1.00 | 23.20 | B | O |
| ATOM | 2091 | N | GLY | B | 212 | 47.940 | 19.180 | 126.044 | 1.00 | 18.79 | B | N |
| ATOM | 2092 | CA | GLY | B | 212 | 48.420 | 17.877 | 125.623 | 1.00 | 17.78 | B | C |
| ATOM | 2093 | C | GLY | B | 212 | 47.925 | 16.758 | 126.521 | 1.00 | 18.87 | B | C |
| ATOM | 2094 | O | GLY | B | 212 | 47.238 | 17.010 | 127.515 | 1.00 | 19.58 | B | O |
| ATOM | 2095 | N | GLN | B | 391 | 48.268 | 15.519 | 126.181 | 1.00 | 19.71 | B | N |
| ATOM | 2096 | CA | GLN | B | 391 | 47.828 | 14.384 | 126.978 | 1.00 | 24.27 | B | C |
| ATOM | 2097 | CB | GLN | B | 391 | 48.024 | 13.073 | 126.200 | 1.00 | 26.88 | B | C |
| ATOM | 2098 | CG | GLN | B | 391 | 49.371 | 12.436 | 126.354 | 1.00 | 36.19 | B | C |
| ATOM | 2099 | CD | GLN | B | 391 | 50.489 | 13.421 | 126.157 | 1.00 | 42.59 | B | C |
| ATOM | 2100 | OE1 | GLN | B | 391 | 50.568 | 14.090 | 125.131 | 1.00 | 51.17 | B | O |
| ATOM | 2101 | NE2 | GLN | B | 391 | 51.368 | 13.520 | 127.144 | 1.00 | 50.82 | B | N |
| ATOM | 2102 | C | GLN | B | 391 | 48.582 | 14.369 | 128.311 | 1.00 | 22.67 | B | C |
| ATOM | 2103 | O | GLN | B | 391 | 49.614 | 15.024 | 128.456 | 1.00 | 19.97 | B | O |
| ATOM | 2104 | N | GLY | B | 392 | 48.050 | 13.635 | 129.284 | 1.00 | 22.31 | B | N |
| ATOM | 2105 | CA | GLY | B | 392 | 48.672 | 13.586 | 130.592 | 1.00 | 20.72 | B | C |
| ATOM | 2106 | C | GLY | B | 392 | 48.050 | 14.651 | 131.473 | 1.00 | 19.93 | B | C |
| ATOM | 2107 | O | GLY | B | 392 | 47.238 | 15.452 | 131.010 | 1.00 | 20.51 | B | O |
| ATOM | 2108 | N | TYR | B | 393 | 48.433 | 14.672 | 132.744 | 1.00 | 20.46 | B | N |
| ATOM | 2109 | CA | TYR | B | 393 | 47.896 | 15.643 | 133.692 | 1.00 | 18.29 | B | C |
| ATOM | 2110 | CB | TYR | B | 393 | 47.421 | 14.924 | 134.962 | 1.00 | 20.08 | B | C |
| ATOM | 2111 | CG | TYR | B | 393 | 46.248 | 13.989 | 134.746 | 1.00 | 16.50 | B | C |
| ATOM | 2112 | CD1 | TYR | B | 393 | 44.957 | 14.490 | 134.602 | 1.00 | 17.97 | B | C |
| ATOM | 2113 | CB1 | TYR | B | 393 | 43.871 | 13.647 | 134.397 | 1.00 | 16.85 | B | C |
| ATOM | 2114 | CD2 | TYR | B | 393 | 46.429 | 12.610 | 134.675 | 1.00 | 18.06 | B | C |
| ATOM | 2115 | CE2 | TYR | B | 393 | 45.342 | 11.747 | 134.467 | 1.00 | 17.10 | B | C |
| ATOM | 2116 | CZ | TYR | B | 393 | 44.064 | 12.281 | 134.329 | 1.00 | 18.56 | B | C |
| ATOM | 2117 | OH | TYR | B | 393 | 42.974 | 11.463 | 134.100 | 1.00 | 18.94 | B | O |
| ATOM | 2118 | C | TYR | B | 393 | 48.964 | 16.653 | 134.050 | 1.00 | 17.76 | B | C |
| ATOM | 2119 | O | TYR | B | 393 | 50.138 | 16.304 | 134.130 | 1.00 | 22.05 | B | O |
| ATOM | 2120 | N | SER | B | 394 | 48.560 | 17.901 | 134.271 | 1.00 | 17.69 | B | N |
| ATOM | 2121 | CA | SER | B | 394 | 49.505 | 18.955 | 134.627 | 1.00 | 18.43 | B | C |
| ATOM | 2122 | CB | SER | B | 394 | 48.864 | 20.331 | 134.464 | 1.00 | 15.87 | B | C |
| ATOM | 2123 | OG | SER | B | 394 | 49.799 | 21.357 | 134.756 | 1.00 | 17.63 | B | O |
| ATOM | 2124 | C | SER | B | 394 | 49.974 | 18.806 | 136.066 | 1.00 | 19.32 | B | C |
| ATOM | 2125 | O | SER | B | 394 | 49.185 | 18.953 | 136.997 | 1.00 | 23.73 | B | O |
| ATOM | 2126 | N | LEU | B | 395 | 51.253 | 18.512 | 136.255 | 1.00 | 18.56 | B | N |
| ATOM | 2127 | CA | LEU | B | 395 | 51.785 | 18.373 | 137.604 | 1.00 | 17.72 | B | C |
| ATOM | 2128 | CB | LEU | B | 395 | 53.265 | 17.972 | 137.556 | 1.00 | 16.48 | B | C |
| ATOM | 2129 | CG | LEU | B | 395 | 53.993 | 17.751 | 138.889 | 1.00 | 15.25 | B | C |
| ATOM | 2130 | CD1 | LEU | B | 395 | 53.311 | 16.631 | 139.670 | 1.00 | 13.74 | B | C |
| ATOM | 2131 | CD2 | LEU | B | 395 | 55.457 | 17.409 | 138.628 | 1.00 | 10.40 | B | C |
| ATOM | 2132 | C | LEU | B | 395 | 51.624 | 19.710 | 138.329 | 1.00 | 18.62 | B | C |
| ATOM | 2133 | O | LEU | B | 395 | 51.285 | 19.746 | 139.507 | 1.00 | 19.25 | B | O |
| ATOM | 2134 | N | PHE | B | 396 | 51.853 | 20.803 | 137.605 | 1.00 | 16.65 | B | N |
| ATOM | 2135 | CA | PHE | B | 396 | 51.741 | 22.152 | 138.152 | 1.00 | 17.42 | B | C |
| ATOM | 2136 | CB | PHE | B | 396 | 52.035 | 23.181 | 137.057 | 1.00 | 16.61 | B | C |
| ATOM | 2137 | CG | PHE | B | 396 | 51.661 | 24.592 | 137.421 | 1.00 | 17.86 | B | C |
| ATOM | 2138 | CD1 | PUB | B | 396 | 52.306 | 25.261 | 138.456 | 1.00 | 17.66 | B | C |
| ATOM | 2139 | CD2 | PHE | B | 396 | 50.666 | 25.259 | 136.714 | 1.00 | 19.99 | B | C |
| ATOM | 2140 | CE1 | PHE | B | 396 | 51.966 | 26.580 | 138.782 | 1.00 | 17.56 | B | C |
| ATOM | 2141 | CE2 | PUB | B | 396 | 50.319 | 26.573 | 137.030 | 1.00 | 20.09 | B | C |
| ATOM | 2142 | CZ | PHE | B | 396 | 50.974 | 27.236 | 138.070 | 1.00 | 19.48 | B | C |
| ATOM | 2143 | C | PUB | B | 396 | 50.369 | 22.429 | 138.749 | 1.00 | 20.69 | B | C |
| ATOM | 2144 | O | PUB | B | 396 | 50.257 | 22.821 | 139.916 | 1.00 | 20.46 | B | O |
| ATOM | 2145 | N | LEU | B | 397 | 49.329 | 22.234 | 137.942 | 1.00 | 20.63 | B | N |
| ATOM | 2146 | CA | LEU | B | 397 | 47.958 | 22.472 | 138.381 | 1.00 | 19.67 | B | C |
| ATOM | 2147 | CB | LEU | B | 397 | 46.985 | 22.340 | 137.201 | 1.00 | 19.64 | B | C |
| ATOM | 2148 | CG | LEU | B | 397 | 47.038 | 23.424 | 136.122 | 1.00 | 18.58 | B | C |
| ATOM | 2149 | CD1 | LEU | B | 397 | 45.965 | 23.156 | 135.086 | 1.00 | 45.91 | B | C |
| ATOM | 2150 | CD2 | LEU | B | 397 | 46.843 | 24.793 | 136.748 | 1.00 | 18.23 | B | C |
| ATOM | 2151 | C | LEU | B | 397 | 47.512 | 21.550 | 139.510 | 1.00 | 18.44 | B | C |
| ATOM | 2152 | O | LEU | B | 397 | 46.858 | 21.997 | 140.452 | 1.00 | 19.34 | B | O |
| ATOM | 2153 | N | VAL | B | 398 | 47.846 | 20.266 | 139.417 | 1.00 | 17.99 | B | N |
| ATOM | 2154 | CA | VAL | B | 398 | 47.464 | 19.326 | 140.466 | 1.00 | 19.04 | B | C |
| ATOM | 2155 | CB | VAt | B | 398 | 47.733 | 17.866 | 140.043 | 1.00 | 19.35 | B | C |
| ATOM | 2156 | CG1 | VAL | B | 398 | 47.353 | 16.905 | 141.173 | 1.00 | 15.55 | B | C |
| ATOM | 2157 | CG2 | VAL | B | 398 | 46.913 | 17.538 | 138.809 | 1.00 | 19.62 | B | C |
| ATOM | 2158 | C | VAL | B | 398 | 48.211 | 19.629 | 141.773 | 1.00 | 20.64 | B | C |
| ATOM | 2159 | O | VAL | B | 398 | 47.659 | 19.469 | 142.862 | 1.00 | 21.46 | B | O |
| ATOM | 2160 | N | ALA | B | 399 | 49.457 | 20.079 | 141.660 | 1.00 | 18.80 | B | N |
| ATOM | 2161 | CA | ALA | B | 399 | 50.254 | 20.411 | 142.834 | 1.00 | 21.04 | B | C |
| ATOM | 2162 | CB | ALA | B | 399 | 51.701 | 20.743 | 142.421 | 1.00 | 17.73 | B | C |
| ATOM | 2163 | C | ALA | B | 399 | 49.636 | 21.599 | 143.566 | 1.00 | 21.74 | B | C |
| ATOM | 2164 | O | ALA | B | 399 | 49.548 | 21.615 | 144.795 | 1.00 | 22.28 | B | O |
| ATOM | 2165 | N | ALA | B | 400 | 49.210 | 22.597 | 142.802 | 1.00 | 21.01 | B | N |
| ATOM | 2166 | CA | ALA | B | 400 | 48.611 | 23.787 | 143.378 | 1.00 | 20.55 | B | C |
| ATOM | 2167 | CB | ALA | B | 400 | 48.270 | 24.788 | 142.274 | 1.00 | 19.74 | B | C |
| ATOM | 2168 | C | ALA | B | 400 | 47.363 | 23.413 | 144.168 | 1.00 | 22.57 | B | C |
| ATOM | 2169 | O | ALA | B | 400 | 47.090 | 23.992 | 145.220 | 1.00 | 23.97 | B | O |
| ATOM | 2170 | N | HIS | B | 401 | 46.612 | 22.442 | 143.658 | 1.00 | 22.90 | B | N |
| ATOM | 2171 | CA | HIS | B | 401 | 45.397 | 21.980 | 144.321 | 1.00 | 23.48 | B | C |
| ATOM | 2172 | CB | HIS | B | 401 | 44.616 | 21.041 | 143.395 | 1.00 | 22.18 | B | C |
| ATOM | 2173 | CG | HIS | B | 401 | 43.328 | 20.544 | 143.976 | 1.00 | 23.80 | B | C |
| ATOM | 2174 | CD2 | HIS | B | 401 | 43.044 | 19.429 | 144.691 | 1.00 | 21.37 | B | C |
| ATOM | 2175 | ND1 | HIS | B | 401 | 42.138 | 21.230 | 143.847 | 1.00 | 23.81 | B | N |
| ATOM | 2176 | CE1 | HIS | B | 401 | 41.177 | 20.557 | 144.454 | 1.00 | 24.18 | B | C |
| ATOM | 2177 | NE2 | HIS | B | 401 | 41.699 | 19.461 | 144.976 | 1.00 | 21.29 | B | N |
| ATOM | 2178 | C | HIS | B | 401 | 45.763 | 21.246 | 145.615 | 1.00 | 24.30 | B | C |
| ATOM | 2179 | O | HIS | B | 401 | 45.192 | 21.519 | 146.673 | 1.00 | 24.53 | B | O |
| ATOM | 2180 | N | GLU | B | 402 | 46.714 | 20.317 | 145.529 | 1.00 | 23.82 | B | N |
| ATOM | 2181 | CA | GLU | B | 402 | 47.150 | 19.566 | 146.708 | 1.00 | 22.77 | B | C |
| ATOM | 2182 | CB | GLU | B | 402 | 48.217 | 18.530 | 146.341 | 1.00 | 22.35 | B | C |
| ATOM | 2183 | CG | GLU | B | 402 | 47.744 | 17.435 | 145.406 | 1.00 | 21.55 | B | C |
| ATOM | 2184 | CD | GLU | B | 402 | 46.298 | 17.071 | 145.636 | 1.00 | 21.21 | B | C |
| ATOM | 2185 | OE1 | GLU | B | 402 | 45.914 | 16.813 | 146.790 | 1.00 | 23.03 | B | O |
| ATOM | 2186 | OE2 | GLU | B | 402 | 45.539 | 17.043 | 144.654 | 1.00 | 26.51 | B | O |
| ATOM | 2187 | C | GLU | B | 402 | 47.710 | 20.486 | 147.785 | 1.00 | 22.14 | B | C |
| ATOM | 2188 | O | GLU | B | 402 | 47.431 | 20.301 | 148.966 | 1.00 | 24.45 | B | O |
| ATOM | 2189 | N | PHE | B | 403 | 48.504 | 21.473 | 147.382 | 1.00 | 22.87 | B | N |
| ATOM | 2190 | CA | PHE | B | 403 | 49.079 | 22.396 | 148.348 | 1.00 | 23.58 | B | C |
| ATOM | 2191 | CB | PHE | B | 403 | 50.124 | 23.308 | 147.688 | 1.00 | 20.83 | B | C |
| ATOM | 2192 | CG | PHE | B | 403 | 51.322 | 22.571 | 147.140 | 1.00 | 22.36 | B | C |
| ATOM | 2193 | CD1 | PHE | B | 403 | 51.587 | 21.252 | 147.518 | 1.00 | 20.84 | B | C |
| ATOM | 2194 | CD2 | PHE | B | 403 | 52.175 | 23.189 | 146.230 | 1.00 | 18.11 | B | C |
| ATOM | 2195 | CE1 | PHE | B | 403 | 52.682 | 20.560 | 146.989 | 1.00 | 21.54 | B | C |
| ATOM | 2196 | CE2 | PHE | B | 403 | 53.272 | 22.507 | 145.695 | 1.00 | 18.09 | B | C |
| ATOM | 2197 | CZ | PHE | B | 403 | 53.525 | 21.193 | 146.072 | 1.00 | 19.86 | B | C |
| ATOM | 2198 | C | PHE | B | 403 | 47.975 | 23.229 | 148.981 | 1.00 | 25.40 | B | C |
| ATOM | 2199 | O | PHE | B | 403 | 48.155 | 23.801 | 150.055 | 1.00 | 27.88 | B | O |
| ATOM | 2200 | N | GLY | B | 404 | 46.829 | 23.288 | 148.310 | 1.00 | 25.83 | B | N |
| ATOM | 2201 | CA | GLY | B | 404 | 45.703 | 24.038 | 148.833 | 1.00 | 25.16 | B | C |
| ATOM | 2202 | C | GLY | B | 404 | 45.208 | 23.328 | 150.076 | 1.00 | 25.03 | B | C |
| ATOM | 2203 | O | GLY | B | 404 | 44.922 | 23.953 | 151.096 | 1.00 | 24.69 | B | O |
| ATOM | 2204 | N | HIS | B | 405 | 45.110 | 22.008 | 149.980 | 1.00 | 23.03 | B | N |
| ATOM | 2205 | CA | HIS | B | 405 | 44.689 | 21.186 | 151.102 | 1.00 | 23.37 | B | C |
| ATOM | 2206 | CB | HIS | B | 405 | 44.537 | 19.731 | 150.668 | 1.00 | 21.33 | B | C |
| ATOM | 2207 | CG | HIS | B | 405 | 43.325 | 19.474 | 149.834 | 1.00 | 26.14 | B | C |
| ATOM | 2208 | CD2 | HIS | B | 405 | 43.164 | 18.781 | 148.683 | 1.00 | 23.33 | B | C |
| ATOM | 2209 | ND1 | HIS | B | 405 | 42.076 | 19.944 | 150.178 | 1.00 | 25.46 | B | N |
| ATOM | 2210 | CE1 | HIS | B | 405 | 41.198 | 19.551 | 149.274 | 1.00 | 26.71 | B | C |
| ATOM | 2211 | NE2 | HIS | B | 405 | 41.833 | 18.844 | 148.357 | 1.00 | 25.66 | B | N |
| ATOM | 2212 | C | HIS | B | 405 | 45.736 | 21.262 | 152.215 | 1.00 | 26.13 | B | C |
| ATOM | 2213 | O | HIS | B | 405 | 45.407 | 21.507 | 153.371 | 1.00 | 27.66 | B | O |
| ATOM | 2214 | N | ALA | B | 406 | 46.998 | 21.053 | 151.850 | 1.00 | 24.97 | B | N |
| ATOM | 2215 | CA | ALA | B | 406 | 48.099 | 21.082 | 152.802 | 1.00 | 25.23 | B | C |
| ATOM | 2216 | CB | ALA | B | 406 | 49.424 | 20.868 | 152.075 | 1.00 | 25.56 | B | C |
| ATOM | 2217 | C | ALA | B | 406 | 48.150 | 22.374 | 153.606 | 1.00 | 24.96 | B | C |
| ATOM | 2218 | O | ALA | B | 406 | 48.822 | 22.433 | 154.634 | 1.00 | 24.90 | B | O |
| ATOM | 2219 | N | LEU | B | 407 | 47.450 | 23.407 | 153.139 | 1.00 | 25.75 | B | N |
| ATOM | 2220 | CA | LEU | B | 407 | 47.424 | 24.683 | 153.849 | 1.00 | 26.02 | B | C |
| ATOM | 2221 | CB | LEU | B | 407 | 47.613 | 25.856 | 152.887 | 1.00 | 24.31 | B | C |
| ATOM | 2222 | CG | LEU | B | 407 | 48.951 | 25.959 | 152.146 | 1.00 | 27.21 | B | C |
| ATOM | 2223 | CD1 | LEU | B | 407 | 48.960 | 27.227 | 151.301 | 1.00 | 24.05 | B | C |
| ATOM | 2224 | CD2 | LEU | B | 407 | 50.110 | 25.979 | 153.132 | 1.00 | 25.82 | B | C |
| ATOM | 2225 | C | LEU | B | 407 | 46.121 | 24.869 | 154.627 | 1.00 | 27.45 | B | C |
| ATOM | 2226 | O | LEU | B | 407 | 45.947 | 25.868 | 155.323 | 1.00 | 28.27 | B | O |
| ATOM | 2227 | N | GLY | B | 408 | 45.208 | 23.910 | 154.504 | 1.00 | 27.89 | B | N |
| ATOM | 2228 | CA | GLY | B | 408 | 43.957 | 23.998 | 155.234 | 1.00 | 30.11 | B | C |
| ATOM | 2229 | C | GLY | B | 408 | 42.673 | 24.146 | 154.437 | 1.00 | 31.82 | B | C |
| ATOM | 2230 | O | GLY | B | 408 | 41.588 | 23.994 | 154.989 | 1.00 | 34.34 | B | O |
| ATOM | 2231 | N | LEU | B | 409 | 42.772 | 24.438 | 153.147 | 1.00 | 33.15 | B | N |
| ATOM | 2232 | CA | LEU | B | 409 | 41.575 | 24.601 | 152.325 | 1.00 | 32.57 | B | C |
| ATOM | 2233 | CB | LEU | B | 409 | 41.925 | 25.260 | 150.991 | 1.00 | 31.21 | B | C |
| ATOM | 2234 | CG | LEU | B | 409 | 42.400 | 26.711 | 151.009 | 1.00 | 32.82 | B | C |
| ATOM | 2235 | CD1 | LEU | B | 409 | 42.551 | 27.189 | 149.581 | 1.00 | 31.72 | B | C |
| ATOM | 2236 | CD2 | LEU | B | 409 | 41.397 | 27.589 | 151.747 | 1.00 | 33.90 | B | C |
| ATOM | 2237 | C | LEU | B | 409 | 40.826 | 23.305 | 152.034 | 1.00 | 33.31 | B | C |
| ATOM | 2238 | O | LEU | B | 409 | 41.427 | 22.250 | 151.850 | 1.00 | 32.51 | B | O |
| ATOM | 2239 | N | ASP | B | 410 | 39.500 | 23.396 | 152.003 | 1.00 | 35.18 | B | N |
| ATOM | 2240 | CA | ASP | B | 410 | 38.660 | 22.248 | 151.684 | 1.00 | 35.77 | B | C |
| ATOM | 2241 | CB | ASP | B | 410 | 37.422 | 22.186 | 152.595 | 1.00 | 42.82 | B | C |
| ATOM | 2242 | CG | ASP | B | 410 | 37.767 | 21.848 | 154.046 | 1.00 | 51.30 | B | C |
| ATOM | 2243 | OD1 | ASP | B | 410 | 38.706 | 21.050 | 154.273 | 1.00 | 53.65 | B | O |
| ATOM | 2244 | OD2 | ASP | B | 410 | 37.084 | 22.365 | 154.963 | 1.00 | 54.35 | B | O |
| ATOM | 2245 | C | ASP | B | 410 | 38.213 | 22.438 | 150.232 | 1.00 | 34.97 | B | C |
| ATOM | 2246 | O | ASP | B | 410 | 38.553 | 23.442 | 149.592 | 1.00 | 31.85 | B | O |
| ATOM | 2247 | N | HIS | B | 411 | 37.452 | 21.482 | 149.713 | 1.00 | 32.49 | B | N |
| ATOM | 2248 | CA | HIS | B | 411 | 36.967 | 21.565 | 148.342 | 1.00 | 33.78 | B | C |
| ATOM | 2249 | CB | HIS | B | 411 | 36.312 | 20.248 | 147.944 | 1.00 | 30.30 | B | C |
| ATOM | 2250 | CG | HIS | B | 411 | 37.294 | 19.186 | 147.573 | 1.00 | 29.54 | B | C |
| ATOM | 2251 | CD2 | HIS | B | 411 | 38.532 | 19.270 | 147.036 | 1.00 | 27.76 | B | C |
| ATOM | 2252 | ND1 | HIS | B | 411 | 37.039 | 17.843 | 147.741 | 1.00 | 28.79 | B | N |
| ATOM | 2253 | GE1 | HIS | B | 411 | 38.079 | 17.144 | 147.325 | 1.00 | 28.07 | B | C |
| ATOM | 2254 | NE2 | HIS | B | 411 | 38.998 | 17.986 | 146.892 | 1.00 | 31.97 | B | N |
| ATOM | 2255 | C | HIS | B | 411 | 35.985 | 22.704 | 148.128 | 1.00 | 35.95 | B | C |
| ATOM | 2256 | O | HIS | B | 411 | 35.224 | 23.062 | 149.028 | 1.00 | 38.94 | B | O |
| ATOM | 2257 | N | SER | B | 412 | 36.013 | 23.274 | 146.927 | 1.00 | 37.78 | B | N |
| ATOM | 2258 | CA | SER | B | 412 | 35.115 | 24.365 | 146.566 | 1.00 | 38.30 | B | C |
| ATOM | 2259 | CB | SER | B | 412 | 35.846 | 25.417 | 145.726 | 1.00 | 37.72 | B | C |
| ATOM | 2260 | OG | SER | B | 412 | 34.953 | 26.426 | 145.281 | 1.00 | 34.94 | B | O |
| ATOM | 2261 | C | SER | B | 412 | 33.957 | 23.800 | 145.763 | 1.00 | 39.44 | B | C |
| ATOM | 2262 | O | SER | B | 412 | 34.050 | 22.703 | 145.213 | 1.00 | 38.62 | B | O |
| ATOM | 2263 | N | SER | B | 413 | 32.862 | 24.553 | 145.715 | 1.00 | 41.65 | B | N |
| ATOM | 2264 | CA | SER | B | 413 | 31.672 | 24.149 | 144.973 | 1.00 | 43.35 | B | C |
| ATOM | 2265 | CB | SER | B | 413 | 30.414 | 24.431 | 145.806 | 1.00 | 43.75 | B | C |
| ATOM | 2266 | OG | SER | B | 413 | 30.416 | 25.759 | 146.311 | 1.00 | 45.44 | B | O |
| ATOM | 2267 | C | SER | B | 413 | 31.620 | 24.910 | 143.644 | 1.00 | 43.39 | B | C |
| ATOM | 2268 | O | SER | B | 413 | 30.789 | 24.627 | 142.782 | 1.00 | 43.76 | B | O |
| ATOM | 2269 | N | VAL | B | 414 | 32.520 | 25.878 | 143.495 | 1.00 | 43.36 | B | N |
| ATOM | 2270 | CA | VAL | B | 414 | 32.613 | 26.679 | 142.282 | 1.00 | 45.01 | B | C |
| ATOM | 2271 | CB | VAL | B | 414 | 33.330 | 28.015 | 142.569 | 1.00 | 43.92 | B | C |
| ATOM | 2272 | CG1 | VAL | B | 414 | 33.395 | 28.862 | 141.307 | 1.00 | 42.90 | B | C |
| ATOM | 2273 | CG2 | VAL | B | 414 | 32.606 | 28.761 | 143.691 | 1.00 | 42.04 | B | C |
| ATOM | 2274 | C | VAt | B | 414 | 33.418 | 25.873 | 141.257 | 1.00 | 49.61 | B | C |
| ATOM | 2275 | O | VAt | B | 414 | 34.639 | 25.731 | 141.384 | 1.00 | 50.35 | B | O |
| ATOM | 2276 | N | PRO | B | 415 | 32.739 | 25.326 | 140.231 | 1.00 | 53.34 | B | N |
| ATOM | 2277 | CD | PRO | B | 415 | 31.281 | 25.415 | 140.033 | 1.00 | 54.43 | B | C |
| ATOM | 2278 | CA | PRO | B | 415 | 33.348 | 24.518 | 139.166 | 1.00 | 54.30 | B | C |
| ATOM | 2279 | CB | PRO | B | 415 | 32.200 | 24.343 | 138.173 | 1.00 | 56.11 | B | C |
| ATOM | 2280 | CG | PRO | B | 415 | 31.014 | 24.261 | 139.078 | 1.00 | 55.46 | B | C |
| ATOM | 2281 | C | PRO | B | 415 | 34.614 | 25.060 | 138.505 | 1.00 | 52.96 | B | C |
| ATOM | 2282 | O | PRO | B | 415 | 35.396 | 24.287 | 137.955 | 1.00 | 54.37 | B | O |
| ATOM | 2283 | N | GLU | B | 416 | 34.818 | 26.373 | 138.544 | 1.00 | 50.37 | B | N |
| ATOM | 2284 | CA | GLU | B | 416 | 36.016 | 26.946 | 137.940 | 1.00 | 48.82 | B | C |
| ATOM | 2285 | CB | GLU | B | 416 | 35.662 | 28.135 | 137.041 | 1.00 | 53.26 | B | C |
| ATOM | 2286 | CG | GLU | B | 416 | 34.714 | 29.151 | 137.651 | 1.00 | 58.55 | B | C |
| ATOM | 2287 | CD | GLU | B | 416 | 33.266 | 28.900 | 137.270 | 1.00 | 61.82 | B | C |
| ATOM | 2288 | OE1 | GLU | B | 416 | 32.399 | 29.699 | 137.684 | 1.00 | 62.91 | B | O |
| ATOM | 2289 | OE2 | GLU | B | 416 | 32.994 | 27.907 | 136.556 | 1.00 | 63.31 | B | O |
| ATOM | 2290 | C | GLU | B | 416 | 37.059 | 27.369 | 138.971 | 1.00 | 45.83 | B | C |
| ATOM | 2291 | O | GLU | B | 416 | 38.023 | 28.060 | 138.646 | 1.00 | 45.77 | B | O |
| ATOM | 2292 | N | ALA | B | 417 | 36.859 | 26.964 | 140.220 | 1.00 | 40.82 | B | N |
| ATOM | 2293 | CA | ALA | B | 417 | 37.811 | 27.283 | 141.278 | 1.00 | 37.74 | B | C |
| ATOM | 2294 | CB | ALA | B | 417 | 37.105 | 27.337 | 142.632 | 1.00 | 33.86 | B | C |
| ATOM | 2295 | C | ALA | B | 417 | 38.867 | 26.181 | 141.281 | 1.00 | 36.14 | B | C |
| ATOM | 2296 | O | ALA | B | 417 | 38.561 | 25.018 | 140.984 | 1.00 | 34.99 | B | O |
| ATOM | 2297 | N | LEU | B | 418 | 40.107 | 26.543 | 141.605 | 1.00 | 34.03 | B | N |
| ATOM | 2298 | CA | LEU | B | 418 | 41.191 | 25.569 | 141.648 | 1.00 | 30.71 | B | C |
| ATOM | 2299 | CB | LEU | B | 418 | 42.502 | 26.248 | 142.048 | 1.00 | 31.54 | B | C |
| ATOM | 2300 | CG | LEU | B | 418 | 43.718 | 25.338 | 142.279 | 1.00 | 30.96 | B | C |
| ATOM | 2301 | CD1 | LEU | B | 418 | 44.048 | 24.554 | 141.013 | 1.00 | 28.41 | B | C |
| ATOM | 2302 | CD2 | LEU | B | 418 | 44.908 | 26.190 | 142.704 | 1.00 | 29.00 | B | C |
| ATOM | 2303 | C | LEU | B | 418 | 40.886 | 24.445 | 142.629 | 1.00 | 30.37 | B | C |
| ATOM | 2304 | O | LEU | B | 418 | 41.261 | 23.295 | 142.402 | 1.00 | 31.03 | B | O |
| ATOM | 2305 | N | MET | B | 419 | 40.197 | 24.772 | 143.719 | 1.00 | 29.50 | B | N |
| ATOM | 2306 | CA | MET | B | 419 | 39.871 | 23.764 | 144.717 | 1.00 | 30.42 | B | C |
| ATOM | 2307 | CB | MET | B | 419 | 39.657 | 24.418 | 146.085 | 1.00 | 31.33 | B | C |
| ATOM | 2308 | CG | MET | B | 419 | 40.933 | 24.997 | 146.691 | 1.00 | 31.92 | B | C |
| ATOM | 2309 | SD | MET | B | 419 | 42.360 | 23.881 | 146.543 | 1.00 | 33.33 | B | S |
| ATOM | 2310 | CE | MET | B | 419 | 41.836 | 22.484 | 147.532 | 1.00 | 27.72 | B | C |
| ATOM | 2311 | C | MET | B | 419 | 38.688 | 22.862 | 144.381 | 1.00 | 30.21 | B | C |
| ATOM | 2312 | O | MET | B | 419 | 38.301 | 22.021 | 145.193 | 1.00 | 30.07 | B | O |
| ATOM | 2313 | N | TYR | B | 420 | 38.119 | 23.026 | 143.190 | 1.00 | 31.40 | B | N |
| ATOM | 2314 | CA | TYR | B | 420 | 37.006 | 22.180 | 142.769 | 1.00 | 31.95 | B | C |
| ATOM | 2315 | CB | TYR | B | 420 | 36.550 | 22.570 | 141.356 | 1.00 | 36.57 | B | C |
| ATOM | 2316 | CG | TYR | B | 420 | 35.227 | 21.951 | 140.953 | 1.00 | 41.51 | B | C |
| ATOM | 2317 | CD1 | TYR | B | 420 | 34.035 | 22.330 | 141.577 | 1.00 | 43.15 | B | C |
| ATOM | 2318 | CE1 | TYR | B | 420 | 32.824 | 21.719 | 141.250 | 1.00 | 43.91 | B | C |
| ATOM | 2319 | CD2 | TYR | B | 420 | 35.173 | 20.947 | 139.986 | 1.00 | 42.71 | B | C |
| ATOM | 2320 | CE2 | TYR | B | 420 | 33.971 | 20.330 | 139.655 | 1.00 | 43.77 | B | C |
| ATOM | 2321 | CZ | TYR | B | 420 | 32.803 | 20.717 | 140.290 | 1.00 | 44.73 | B | C |
| ATOM | 2322 | OH | TYR | B | 420 | 34.628 | 20.080 | 139.969 | 1.00 | 45.83 | B | O |
| ATOM | 2323 | C | TYR | B | 420 | 37.558 | 20.749 | 142.790 | 1.00 | 31.80 | B | C |
| ATOM | 2324 | O | TYR | B | 420 | 38.658 | 20.498 | 142.310 | 1.00 | 35.03 | B | O |
| ATOM | 2325 | N | PRO | B | 421 | 36.797 | 19.792 | 143.340 | 1.00 | 31.81 | B | N |
| ATOM | 2326 | CD | PRO | B | 421 | 35.430 | 20.002 | 143.846 | 1.00 | 31.47 | B | C |
| ATOM | 2327 | CA | PRO | B | 421 | 37.177 | 18.376 | 143.455 | 1.00 | 31.76 | B | C |
| ATOM | 2328 | CB | PRO | B | 421 | 36.033 | 17.783 | 144.277 | 1.00 | 32.37 | B | C |
| ATOM | 2329 | CG | PRO | B | 421 | 34.869 | 18.597 | 143.833 | 1.00 | 31.84 | B | C |
| ATOM | 2330 | C | PRO | B | 421 | 37.462 | 17.536 | 142.212 | 1.00 | 32.82 | B | C |
| ATOM | 2331 | O | PRO | B | 421 | 37.890 | 16.383 | 142.329 | 1.00 | 33.44 | B | O |
| ATOM | 2332 | N | MET | B | 422 | 37.225 | 18.078 | 141.028 | 1.00 | 34.03 | B | N |
| ATOM | 2333 | CA | MET | B | 422 | 37.469 | 17.303 | 139.819 | 1.00 | 34.21 | B | C |
| ATOM | 2334 | CB | MET | B | 422 | 36.156 | 17.127 | 139.050 | 1.00 | 40.49 | B | C |
| ATOM | 2335 | CG | MET | B | 422 | 35.093 | 16.379 | 139.863 | 1.00 | 46.52 | B | C |
| ATOM | 2336 | SD | MET | B | 422 | 33.548 | 16.039 | 138.978 | 1.00 | 54.83 | B | S |
| ATOM | 2337 | CE | MET | B | 422 | 33.899 | 14.421 | 138.287 | 1.00 | 48.46 | B | C |
| ATOM | 2338 | C | MET | B | 422 | 38.520 | 17.987 | 138.965 | 1.00 | 30.07 | B | C |
| ATOM | 2339 | O | MET | B | 422 | 38.610 | 19.208 | 138.960 | 1.00 | 27.30 | B | O |
| ATOM | 2340 | N | TYR | B | 423 | 39.328 | 17.205 | 138.256 | 1.00 | 30.57 | B | N |
| ATOM | 2341 | CA | TYR | B | 423 | 40.386 | 17.789 | 137.423 | 1.00 | 30.47 | B | C |
| ATOM | 2342 | CB | TYR | B | 423 | 41.432 | 16.730 | 137.066 | 1.00 | 28.25 | B | C |
| ATOM | 2343 | CG | TYR | B | 423 | 42.541 | 17.261 | 136.179 | 1.00 | 28.83 | B | C |
| ATOM | 2344 | CD1 | TYR | B | 423 | 43.549 | 18.077 | 136.695 | 1.00 | 26.05 | B | C |
| ATOM | 2345 | CE1 | TYR | B | 423 | 44.547 | 18.607 | 135.866 | 1.00 | 26.97 | B | C |
| ATOM | 2346 | CD2 | TYR | B | 423 | 42.557 | 16.984 | 134.809 | 1.00 | 29.17 | B | C |
| ATOM | 2347 | CE2 | TYR | B | 423 | 43.548 | 17.509 | 133.973 | 1.00 | 28.79 | B | C |
| ATOM | 2348 | GZ | TYR | B | 423 | 44.537 | 18.319 | 134.508 | 1.00 | 27.46 | B | C |
| ATOM | 2349 | OH | TYR | B | 423 | 45.499 | 18.848 | 133.679 | 1.00 | 29.00 | B | O |
| ATOM | 2350 | C | TYR | B | 423 | 39.891 | 18.450 | 136.132 | 1.00 | 29.81 | B | C |
| ATOM | 2351 | O | TYR | B | 423 | 38.997 | 17.941 | 135.456 | 1.00 | 28.16 | B | O |
| ATOM | 2352 | N | ALA | B | 424 | 40.496 | 19.583 | 135.795 | 1.00 | 30.84 | B | N |
| ATOM | 2353 | CA | ALA | B | 424 | 40.155 | 20.320 | 134.582 | 1.00 | 30.74 | B | C |
| ATOM | 2354 | CB | ALA | B | 424 | 38.891 | 21.137 | 134.809 | 1.00 | 30.80 | B | C |
| ATOM | 2355 | C | ALA | B | 424 | 41.316 | 21.241 | 134.201 | 1.00 | 31.98 | B | C |
| ATOM | 2356 | O | ALA | B | 424 | 41.662 | 22.147 | 134.960 | 1.00 | 30.87 | B | O |
| ATOM | 2357 | N | PHE | B | 425 | 41.916 | 21.016 | 133.030 | 1.00 | 32.65 | B | N |
| ATOM | 2358 | CA | PHE | B | 425 | 43.037 | 21.847 | 132.587 | 1.00 | 32.21 | B | C |
| ATOM | 2359 | CB | PHE | B | 425 | 43.761 | 21.219 | 131.387 | 1.00 | 29.42 | B | C |
| ATOM | 2360 | CG | PHE | B | 425 | 44.871 | 22.085 | 130.837 | 1.00 | 28.07 | B | C |
| ATOM | 2361 | CD1 | PHE | B | 425 | 44.635 | 22.967 | 129.783 | 1.00 | 26.46 | B | C |
| ATOM | 2362 | CD2 | PHE | B | 425 | 46.130 | 22.075 | 131.427 | 1.00 | 26.10 | B | C |
| ATOM | 2363 | GE1 | PHE | B | 425 | 45.636 | 23.828 | 129.334 | 1.00 | 26.71 | B | C |
| ATOM | 2364 | CE2 | PHE | B | 425 | 47.138 | 22.933 | 130.986 | 1.00 | 23.65 | B | C |
| ATOM | 2365 | CZ | PHE | B | 425 | 46.890 | 23.811 | 129.941 | 1.00 | 24.65 | B | C |
| ATOM | 2366 | C | PHE | B | 425 | 42.612 | 23.261 | 132.226 | 1.00 | 32.08 | B | C |
| ATOM | 2367 | O | PHE | B | 425 | 41.517 | 23.474 | 131.723 | 1.00 | 35.15 | B | O |
| ATOM | 2368 | N | THR | B | 426 | 43.489 | 24.226 | 132.480 | 1.00 | 32.53 | B | N |
| ATOM | 2369 | CA | THR | B | 426 | 43.193 | 25.618 | 132.170 | 1.00 | 31.99 | B | C |
| ATOM | 2370 | CB | THR | B | 426 | 42.377 | 26.297 | 133.309 | 1.00 | 33.76 | B | C |
| ATOM | 2371 | OG1 | THR | B | 426 | 42.153 | 27.674 | 132.979 | 1.00 | 35.23 | B | O |
| ATOM | 2372 | OG2 | TER | B | 426 | 43.132 | 26.236 | 134.638 | 1.00 | 32.87 | B | C |
| ATOM | 2373 | C | THR | B | 426 | 44.470 | 26.415 | 131.960 | 1.00 | 32.40 | B | C |
| ATOM | 2374 | O | THR | B | 426 | 45.515 | 26.082 | 132.515 | 1.00 | 31.41 | B | O |
| ATOM | 2375 | N | GLU | B | 427 | 44.376 | 27.458 | 131.143 | 1.00 | 32.69 | B | N |
| ATOM | 2376 | CA | GLU | B | 427 | 45.505 | 28.338 | 130.877 | 1.00 | 34.12 | B | C |
| ATOM | 2377 | CB | GLU | B | 427 | 45.593 | 28.670 | 129.384 | 1.00 | 36.51 | B | C |
| ATOM | 2378 | CG | GLU | B | 427 | 45.967 | 27.493 | 128.490 | 1.00 | 41.69 | B | C |
| ATOM | 2379 | CD | GLU | B | 427 | 46.103 | 27.888 | 127.024 | 1.00 | 45.50 | B | C |
| ATOM | 2380 | OE1 | GLU | B | 427 | 45.082 | 28.269 | 126.410 | 1.00 | 44.55 | B | O |
| ATOM | 2381 | OE2 | GLU | B | 427 | 47.235 | 27.822 | 126.488 | 1.00 | 47.29 | B | O |
| ATOM | 2382 | C | GLU | B | 427 | 45.237 | 29.605 | 131.680 | 1.00 | 33.67 | B | C |
| ATOM | 2383 | O | GLU | B | 427 | 44.133 | 29.793 | 132.186 | 1.00 | 35.20 | B | O |
| ATOM | 2384 | N | GLY | B | 428 | 46.234 | 30.471 | 131.800 | 1.00 | 33.34 | B | N |
| ATOM | 2385 | CA | GLY | B | 428 | 46.041 | 31.692 | 132.561 | 1.00 | 34.51 | B | C |
| ATOM | 2386 | C | GLY | B | 428 | 46.066 | 31.428 | 134.057 | 1.00 | 36.70 | B | C |
| ATOM | 2387 | O | GLY | B | 428 | 46.241 | 30.281 | 134.475 | 1.00 | 35.95 | B | O |
| ATOM | 2388 | N | PRO | B | 429 | 45.887 | 32.468 | 134.893 | 1.00 | 37.61 | B | N |
| ATOM | 2389 | CD | PRO | B | 429 | 45.427 | 33.803 | 134.473 | 1.00 | 37.68 | B | C |
| ATOM | 2390 | CA | PRO | B | 429 | 45.888 | 32.363 | 136.361 | 1.00 | 35.27 | B | C |
| ATOM | 2391 | CB | PRO | B | 429 | 45.444 | 33.753 | 136.806 | 1.00 | 36.80 | B | C |
| ATOM | 2392 | CG | PRO | B | 429 | 44.594 | 34.224 | 135.655 | 1.00 | 38.82 | B | C |
| ATOM | 2393 | C | PRO | B | 429 | 44.969 | 31.258 | 136.882 | 1.00 | 34.51 | B | C |
| ATOM | 2394 | O | PRO | B | 429 | 43.769 | 31.248 | 136.605 | 1.00 | 35.20 | B | O |
| ATOM | 2395 | N | PRO | B | 430 | 45.533 | 30.312 | 137.648 | 1.00 | 32.84 | B | N |
| ATOM | 2396 | CD | PRO | B | 430 | 46.984 | 30.207 | 137.866 | 1.00 | 30.88 | B | C |
| ATOM | 2397 | CA | PRO | B | 430 | 44.843 | 29.160 | 138.246 | 1.00 | 33.23 | B | C |
| ATOM | 2398 | CB | PRO | B | 430 | 45.997 | 28.284 | 138.747 | 1.00 | 32.15 | B | C |
| ATOM | 2399 | CG | PRO | B | 430 | 47.172 | 28.722 | 137.930 | 1.00 | 32.06 | B | C |
| ATOM | 2400 | C | PRO | B | 430 | 43.870 | 29.482 | 139.381 | 1.00 | 33.86 | B | C |
| ATOM | 2401 | O | PRO | B | 430 | 42.786 | 28.891 | 139.475 | 1.00 | 32.09 | B | O |
| ATOM | 2402 | N | LEU | B | 431 | 44.271 | 30.405 | 140.250 | 1.00 | 34.56 | B | N |
| ATOM | 2403 | CA | LEU | B | 431 | 43.456 | 30.778 | 141.401 | 1.00 | 35.70 | B | C |
| ATOM | 2404 | CB | LEU | B | 431 | 44.300 | 31.550 | 142.419 | 1.00 | 33.79 | B | C |
| ATOM | 2405 | CG | LEU | B | 431 | 45.449 | 30.793 | 143.080 | 1.00 | 33.47 | B | C |
| ATOM | 2406 | CD1 | LEU | B | 431 | 46.049 | 31.651 | 144.190 | 1.00 | 33.46 | B | C |
| ATOM | 2407 | CD2 | LEU | B | 431 | 44.934 | 29.481 | 143.649 | 1.00 | 36.47 | B | C |
| ATOM | 2408 | C | LEU | B | 431 | 42.207 | 31.588 | 141.084 | 1.00 | 36.24 | B | C |
| ATOM | 2409 | O | LEU | B | 431 | 42.271 | 32.661 | 140.479 | 1.00 | 36.37 | B | O |
| ATOM | 2410 | N | HIS | B | 432 | 41.064 | 31.064 | 141.505 | 1.00 | 36.71 | B | N |
| ATOM | 2411 | CA | HIS | B | 432 | 39.811 | 31.760 | 141.299 | 1.00 | 41.25 | B | C |
| ATOM | 2412 | CB | HIS | B | 432 | 38.662 | 30.761 | 141.191 | 1.00 | 40.99 | B | C |
| ATOM | 2413 | CG | HIS | B | 432 | 37.332 | 31.404 | 140.963 | 1.00 | 44.68 | B | C |
| ATOM | 2414 | CD2 | HIS | B | 432 | 36.589 | 31.541 | 139.838 | 1.00 | 44.42 | B | C |
| ATOM | 2415 | ND1 | HIS | B | 432 | 36.626 | 32.031 | 141.967 | 1.00 | 46.47 | B | N |
| ATOM | 2416 | CE1 | HIS | B | 432 | 35.504 | 32.524 | 141.472 | 1.00 | 45.59 | B | C |
| ATOM | 2417 | NB2 | HIS | B | 432 | 35.459 | 32.241 | 140.182 | 1.00 | 46.62 | B | N |
| ATOM | 2418 | C | HIS | B | 432 | 39.613 | 32.670 | 142.507 | 1.00 | 44.37 | B | C |
| ATOM | 2419 | O | HIS | B | 432 | 40.151 | 32.400 | 143.588 | 1.00 | 46.02 | B | 0 |
| ATOM | 2420 | N | LYS | B | 433 | 38.859 | 33.751 | 142.330 | 1.00 | 46.58 | B | N |
| ATOM | 2421 | CA | LYS | B | 433 | 38.627 | 34.678 | 143.428 | 1.00 | 46.70 | B | C |
| ATOM | 2422 | CB | LYS | B | 433 | 37.717 | 35.822 | 142.972 | 1.00 | 51.43 | B | C |
| ATOM | 2423 | CG | LYS | B | 433 | 38.355 | 36.668 | 141.865 | 1.00 | 55.90 | B | C |
| ATOM | 2424 | CD | LYS | B | 433 | 37.589 | 37.956 | 141.584 | 1.00 | 59.77 | B | C |
| ATOM | 2425 | CE | LYS | B | 433 | 38.320 | 38.811 | 140.549 | 1.00 | 61.80 | B | C |
| ATOM | 2426 | NZ | LYS | B | 433 | 37.720 | 40.171 | 140.380 | 1.00 | 62.64 | B | N |
| ATOM | 2427 | C | LYS | B | 433 | 38.062 | 33.973 | 144.658 | 1.00 | 43.89 | B | C |
| ATOM | 2428 | O | LYS | B | 433 | 38.150 | 34.491 | 145.767 | 1.00 | 44.99 | B | O |
| ATOM | 2429 | N | ASP | B | 434 | 37.501 | 32.783 | 144.460 | 1.00 | 40.87 | B | N |
| ATOM | 2430 | CA | ASP | B | 434 | 36.964 | 31.998 | 145.565 | 1.00 | 39.24 | B | C |
| ATOM | 2431 | CB | ASP | B | 434 | 36.032 | 30.905 | 145.038 | 1.00 | 38.77 | B | C |
| ATOM | 2432 | CG | ASP | B | 434 | 35.520 | 29.999 | 146.138 | 1.00 | 39.93 | B | C |
| ATOM | 2433 | OD1 | ASP | B | 434 | 36.053 | 28.882 | 146.286 | 1.00 | 43.55 | B | O |
| ATOM | 2434 | OD2 | ASP | B | 434 | 34.593 | 30.408 | 146.866 | 1.00 | 42.42 | B | O |
| ATOM | 2435 | C | ASP | B | 434 | 38.127 | 31.365 | 146.337 | 1.00 | 40.97 | B | C |
| ATOM | 2436 | O | ASP | B | 434 | 38.102 | 31.288 | 147.571 | 1.00 | 41.38 | B | O |
| ATOM | 2437 | N | ASP | B | 435 | 39.144 | 30.915 | 145.601 | 1.00 | 39.44 | B | N |
| ATOM | 2438 | CA | ASP | B | 435 | 40.324 | 30.305 | 146.204 | 1.00 | 37.18 | B | C |
| ATOM | 2439 | CB | ASP | B | 435 | 41.258 | 29.737 | 145.128 | 1.00 | 36.04 | B | C |
| ATOM | 2440 | CG | ASP | B | 435 | 40.608 | 28.653 | 144.293 | 1.00 | 37.10 | B | C |
| ATOM | 2441 | OD1 | ASP | B | 435 | 40.086 | 27.671 | 144.868 | 1.00 | 35.74 | B | O |
| ATOM | 2442 | OD2 | ASP | B | 435 | 40.632 | 28.779 | 143.050 | 1.00 | 40.79 | B | O |
| ATOM | 2443 | C | ASP | B | 435 | 41.088 | 31.354 | 147.007 | 1.00 | 38.01 | B | C |
| ATOM | 2444 | O | ASP | B | 435 | 41.591 | 31.077 | 148.098 | 1.00 | 39.15 | B | O |
| ATOM | 2445 | N | VAL | B | 436 | 41.178 | 32.560 | 146.459 | 1.00 | 37.34 | B | N |
| ATOM | 2446 | CA | VAL | B | 436 | 41.893 | 33.637 | 147.127 | 1.00 | 37.88 | B | C |
| ATOM | 2447 | CB | VAL | B | 436 | 42.018 | 34.863 | 146.209 | 1.00 | 37.00 | B | C |
| ATOM | 2448 | CG1 | VAL | B | 436 | 42.850 | 35.939 | 146.887 | 1.00 | 37.16 | B | C |
| ATOM | 2449 | CO2 | VAL | B | 436 | 42.654 | 34.454 | 144.893 | 1.00 | 37.45 | B | C |
| ATOM | 2450 | C | VAL | B | 436 | 41.203 | 34.051 | 148.426 | 1.00 | 41.35 | B | C |
| ATOM | 2451 | O | VAL | B | 436 | 41.870 | 34.444 | 149.392 | 1.00 | 40.58 | B | O |
| ATOM | 2452 | N | ASN | B | 437 | 39.872 | 33.964 | 148.445 | 1.00 | 42.61 | B | N |
| ATOM | 2453 | CA | ASN | B | 437 | 39.103 | 34.323 | 149.633 | 1.00 | 42.98 | B | C |
| ATOM | 2454 | CB | ASN | B | 437 | 37.602 | 34.372 | 149.331 | 1.00 | 44.38 | B | C |
| ATOM | 2455 | CG | ASN | B | 437 | 37.199 | 35.607 | 148.564 | 1.00 | 46.07 | B | C |
| ATOM | 2456 | OD1 | ASN | B | 437 | 37.778 | 36.681 | 148.743 | 1.00 | 49.54 | B | O |
| ATOM | 2457 | ND2 | ASN | B | 437 | 36.182 | 35.471 | 147.721 | 1.00 | 46.44 | B | N |
| ATOM | 2458 | C | ASN | B | 437 | 39.341 | 33.309 | 150.732 | 1.00 | 41.97 | B | C |
| ATOM | 2459 | O | ASN | B | 437 | 39.656 | 33.665 | 151.863 | 1.00 | 44.18 | B | O |
| ATOM | 2460 | N | GLY | B | 438 | 39.177 | 32.038 | 150.391 | 1.00 | 42.23 | B | N |
| ATOM | 2461 | CA | GLY | B | 438 | 39.373 | 30.980 | 151.364 | 1.00 | 41.20 | B | C |
| ATOM | 2462 | C | GLY | B | 438 | 40.737 | 31.015 | 152.030 | 1.00 | 40.27 | B | C |
| ATOM | 2463 | O | GLY | B | 438 | 40.851 | 30.821 | 153.241 | 1.00 | 40.43 | B | O |
| ATOM | 2464 | N | 1LE | B | 439 | 41.778 | 31.269 | 151.246 | 1.00 | 39.21 | B | N |
| ATOM | 2465 | CA | ILE | B | 439 | 43.125 | 31.305 | 151.796 | 1.00 | 38.89 | B | C |
| ATOM | 2466 | CB | 1LE | B | 439 | 44.195 | 31.186 | 150.666 | 1.00 | 34.85 | B | C |
| ATOM | 2467 | CG2 | RE | B | 439 | 44.318 | 32.488 | 149.913 | 1.00 | 32.27 | B | C |
| ATOM | 2468 | CG1 | ILE | B | 439 | 45.546 | 30.783 | 151.262 | 1.00 | 34.00 | B | C |
| ATOM | 2469 | CD1 | ILE | B | 439 | 45.558 | 29.373 | 151.853 | 1.00 | 32.09 | B | C |
| ATOM | 2470 | C | ILE | B | 439 | 43.317 | 32.597 | 152.587 | 1.00 | 40.47 | B | C |
| ATOM | 2471 | O | ILE | B | 439 | 44.034 | 32.625 | 153.588 | 1.00 | 41.20 | B | O |
| ATOM | 2472 | N | ARG | B | 440 | 42.654 | 33.658 | 152.134 | 1.00 | 43.75 | B | N |
| ATOM | 2473 | CA | ARG | B | 440 | 42.725 | 34.967 | 152.782 | 1.00 | 44.46 | B | C |
| ATOM | 2474 | CB | ARG | B | 440 | 42.048 | 36.030 | 151.909 | 1.00 | 46.94 | B | C |
| ATOM | 2475 | CG | ARG | B | 440 | 42.937 | 37.185 | 151.471 | 1.00 | 49.56 | B | C |
| ATOM | 2476 | CD | ARG | B | 440 | 43.845 | 36.769 | 150.334 | 1.00 | 54.73 | B | C |
| ATOM | 2477 | NE | ARG | B | 440 | 44.726 | 37.850 | 149.901 | 1.00 | 58.03 | B | N |
| ATOM | 2478 | CZ | ARG | B | 440 | 44.306 | 38.991 | 149.362 | 1.00 | 60.42 | B | C |
| ATOM | 2479 | NH1 | ARG | B | 440 | 43.009 | 39.211 | 149.186 | 1.00 | 60.93 | B | N |
| ATOM | 2480 | NH2 | ARG | B | 440 | 45.188 | 39.910 | 148.988 | 1.00 | 60.50 | B | N |
| ATOM | 2481 | C | ARG | B | 440 | 42.006 | 34.908 | 154.128 | 1.00 | 44.17 | B | C |
| ATOM | 2482 | O | ARG | B | 440 | 42.320 | 35.666 | 155.047 | 1.00 | 43.24 | B | O |
| ATOM | 2483 | N | HIS | B | 441 | 41.033 | 34.004 | 154.221 | 1.00 | 42.88 | B | N |
| ATOM | 2484 | CA | HIS | B | 441 | 40.235 | 33.818 | 155.428 | 1.00 | 43.46 | B | C |
| ATOM | 2485 | CB | HIS | B | 441 | 38.997 | 32.972 | 155.112 | 1.00 | 46.73 | B | C |
| ATOM | 2486 | CG | HIS | B | 441 | 38.118 | 32.713 | 156.297 | 1.00 | 52.22 | B | C |
| ATOM | 2487 | CD2 | HIS | B | 441 | 37.906 | 31.588 | 157.022 | 1.00 | 53.91 | B | C |
| ATOM | 2488 | ND1 | HIS | B | 441 | 37.331 | 33.690 | 156.871 | 1.00 | 54.78 | B | N |
| ATOM | 2489 | CE1 | HIS | B | 441 | 36.673 | 33.178 | 157.896 | 1.00 | 54.20 | B | C |
| ATOM | 2490 | NR2 | HIS | B | 441 | 37.005 | 31.904 | 158.010 | 1.00 | 54.76 | B | N |
| ATOM | 2491 | C | HIS | B | 441 | 41.029 | 33.144 | 156.538 | 1.00 | 42.38 | B | C |
| ATOM | 2492 | O | HIS | B | 441 | 40.835 | 33.448 | 157.713 | 1.00 | 43.43 | B | O |
| ATOM | 2493 | N | LEU | B | 442 | 41.924 | 32.233 | 156.158 | 1.00 | 40.54 | B | N |
| ATOM | 2494 | CA | LEU | B | 442 | 42.747 | 31.495 | 157.118 | 1.00 | 37.84 | B | C |
| ATOM | 2495 | GB | LEU | B | 442 | 43.136 | 30.124 | 156.542 | 1.00 | 36.23 | B | C |
| ATOM | 2496 | CG | LEU | B | 442 | 42.020 | 29.121 | 156.224 | 1.00 | 36.95 | B | C |
| ATOM | 2497 | CD1 | LEU | B | 442 | 42.600 | 27.921 | 155.494 | 1.00 | 37.45 | B | C |
| ATOM | 2498 | CD2 | LEU | B | 442 | 41.337 | 28.676 | 157.507 | 1.00 | 38.67 | B | C |
| ATOM | 2499 | C | LEU | B | 442 | 44.012 | 32.232 | 157.548 | 1.00 | 35.29 | B | C |
| ATOM | 2500 | O | LEU | B | 442 | 44.367 | 32.218 | 158.721 | 1.00 | 37.44 | B | O |
| ATOM | 2501 | N | TYR | B | 443 | 44.693 | 32.872 | 156.605 | 1.00 | 34.38 | B | N |
| ATOM | 2502 | CA | TYR | B | 443 | 45.932 | 33.581 | 156.920 | 1.00 | 36.23 | B | C |
| ATOM | 2503 | GB | TYR | B | 443 | 47.105 | 32.984 | 156.123 | 1.00 | 34.25 | B | C |
| ATOM | 2504 | CG | TYR | B | 443 | 47.261 | 31.490 | 156.300 | 1.00 | 31.93 | B | C |
| ATOM | 2505 | CD1 | TYR | B | 443 | 46.614 | 30.596 | 155.446 | 1.00 | 29.81 | B | C |
| ATOM | 2506 | CE1 | TYR | B | 443 | 46.695 | 29.215 | 155.649 | 1.00 | 28.03 | B | C |
| ATOM | 2507 | CD2 | TYR | B | 443 | 48.004 | 30.968 | 157.366 | 1.00 | 29.17 | B | C |
| ATOM | 2508 | CE2 | TYR | B | 443 | 48.092 | 29.592 | 157.578 | 1.00 | 28.10 | B | C |
| ATOM | 2509 | CZ | TYR | B | 443 | 47.433 | 28.721 | 156.718 | 1.00 | 29.15 | B | C |
| ATOM | 2510 | OH | TYR | B | 443 | 47.496 | 27.360 | 156.932 | 1.00 | 27.85 | B | O |
| ATOM | 2511 | C | TYR | B | 443 | 45.855 | 35.082 | 156.659 | 1.00 | 38.24 | B | C |
| ATOM | 2512 | O | TYR | B | 443 | 46.801 | 35.794 | 157.063 | 1.00 | 40.58 | B | O |
| ATOM | 2513 | OT | TYR | B | 443 | 44.864 | 35.527 | 156.044 | 1.00 | 41.15 | B | O |
| ATOM | 2562 | ZN | ZN | B | 1 | 41.275 | 17.840 | 146.537 | 1.00 | 30.01 | Z | ZN |
| ATOM | 2563 | ZN | ZN | B | 2 | 49.676 | 8.887 | 150.296 | 1.00 | 22.73 | Z | ZN |
| ATOM | 2570 | CA | CA | B | 3 | 54.013 | 17.086 | 157.486 | 1.00 | 25.83 | C | C |
| ATOM | 2571 | CA | CA | B | 4 | 54.046 | 8.303 | 135.827 | 1.00 | 47.76 | C | C |
| ATOM | 2572 | CA | CA | B | 5 | 50.701 | 18.541 | 128.694 | 1.00 | 57.49 | C | C |
| ATOM | 2569 | CA | CA | B | 6 | 45.250 | 7.241 | 139.948 | 1.00 | 21.01 | C | C |
| ATOM | 2574 | CA | CA | B | 7 | 53.492 | 38.165 | 150.097 | 1.00 | 99.62 | C | C |
| ATOM | 2537 | C1 | FRA | B | 2 | 40.988 | 17.843 | 141.282 | 1.00 | 21.42 | M | C |
| ATOM | 2538 | C2 | FRA | B | 2 | 42.202 | 16.892 | 141.164 | 1.00 | 23.04 | M | C |
| ATOM | 2539 | C3 | FRA | B | 2 | 43.397 | 17.813 | 140.945 | 1.00 | 21.68 | M | C |
| ATOM | 2540 | C4 | FRA | B | 2 | 42.395 | 16.088 | 142.486 | 1.00 | 20.53 | M | C |
| ATOM | 2541 | C5 | FRA | B | 2 | 41.287 | 15.047 | 142.944 | 1.00 | 22.68 | M | C |
| ATOM | 2542 | C6 | FRA | B | 2 | 41.674 | 14.384 | 144.328 | 1.00 | 21.04 | M | C |
| ATOM | 2543 | N7 | FRA | B | 2 | 41.894 | 15.346 | 145.441 | 1.00 | 26.66 | M | N |
| ATOM | 2544 | C8 | FRA | B | 2 | 40.878 | 16.003 | 145.917 | 1.00 | 16.04 | M | O |
| ATOM | 2545 | C9 | FRA | B | 2 | 43.101 | 15.578 | 145.982 | 1.00 | 25.43 | M | C |
| ATOM | 2546 | C10 | FRA | B | 2 | 43.304 | 16.343 | 146.865 | 1.00 | 27.78 | M | O |
| ATOM | 2547 | C11 | FRA | B | 2 | 41.103 | 13.852 | 141.935 | 1.00 | 24.21 | M | C |
| ATOM | 2548 | N12 | FRA | B | 2 | 39.910 | 13.731 | 141.312 | 1.00 | 24.87 | M | N |
| ATOM | 2549 | C13 | FRA | B | 2 | 39.558 | 12.636 | 140.307 | 1.00 | 25.69 | M | C |
| ATOM | 2550 | C14 | FRA | B | 2 | 38.064 | 12.068 | 140.550 | 1.00 | 26.41 | M | C |
| ATOM | 25S1 | C15 | FRA | B | 2 | 37.671 | 10.934 | 139.534 | 1.00 | 23.S4 | M | C |
| ATOM | 2S52 | C16 | FRA | B | 2 | 36.936 | 13.161 | 140.404 | 1.00 | 25.14 | M | C |
| ATOM | 2553 | C17 | FRA | B | 2 | 37.918 | 11.501 | 141.991 | 1.00 | 25.52 | M | C |
| ATOM | 2554 | C18 | FRA | B | 2 | 39.789 | 13.241 | 138.843 | 1.00 | 26.03 | M | C |
| ATOM | 2555 | O19 | FRA | B | 2 | 39.414 | 14.401 | 138.550 | 1.00 | 27.42 | M | O |
| ATOM | 2556 | N20 | FRA | B | 2 | 40.416 | 12.393 | 137.981 | 1.00 | 25.15 | M | N |
| ATOM | 2557 | C21 | FRA | B | 2 | 40.752 | 12.702 | 136.573 | 1.00 | 30.05 | M | C |
| ATOM | 2558 | 022 | FRA | B | 2 | 42.049 | 13.051 | 141.732 | 1.00 | 25.04 | M | O |
| ATOM | 2576 | O | HOE | W | 1 | 53.372 | 24.644 | 125.318 | 1.00 | 1.00 | W | O |
| ATOM | 2577 | O | HON | W | 2 | 74.083 | 20.414 | 120.913 | 1.00 | 21.76 | W | O |
| ATOM | 2578 | O | HOH | W | 3 | 49.364 | 12.911 | 104.335 | 1.00 | 26.01 | W | O |
| ATOM | 2579 | O | HON | W | 4 | 52.1M | 16.494 | 124.475 | 1.00 | 14.30 | W | O |
| ATOM | 2580 | O | HOH | W | 5 | 58.587 | 7.014 | 115.292 | 1.00 | 27.11 | W | O |
| ATOM | 2581 | O | HOH | W | 6 | 75.605 | 18.864 | 112.249 | 1.00 | 18.56 | W | O |
| ATOM | 2582 | O | HOH | W | 7 | 65.295 | 7.257 | 120.405 | 1.00 | 1.00 | W | O |
| ATOM | 2583 | O | HOH | W | 8 | 57.185 | 8.107 | 113.191 | 1.00 | 17.03 | W | O |
| ATOM | 2584 | O | HOH | W | 9 | 64.469 | 12.077 | 98.818 | 1.00 | 47.82 | W | O |
| ATOM | 2585 | O | HOH | W | 10 | 51.666 | 21.908 | 124.829 | 1.00 | 15.33 | W | O |
| ATOM | 2586 | O | HOH | W | 11 | 67.629 | 24.639 | 127.665 | 1.00 | 20.55 | W | O |
| ATOM | 2587 | O | HOH | W | 12 | 67.365 | 14.751 | 108.625 | 1.00 | 21.02 | W | O |
| ATOM | 2588 | O | HOH | W | 13 | 52.957 | 12.041 | 116.137 | 1.00 | 15.20 | W | O |
| ATOM | 2589 | O | HOH | W | 14 | 51.253 | 9.646 | 116.663 | 1.00 | 29.59 | W | O |
| ATOM | 2590 | O | HOH | W | 15 | 48.380 | 16.148 | 113.137 | 1.00 | 26.43 | W | O |
| ATOM | 2591 | O | HOH | W | 16 | 49.177 | 9.855 | 111.408 | 1.00 | 25.21 | W | O |
| ATOM | 2592 | O | HOH | W | 17 | 56.870 | 18.582 | 90.415 | 1.00 | 50.62 | W | O |
| ATOM | 2593 | O | HON | W | 18 | 56.995 | 17.921 | 96.140 | 1.00 | 26.85 | W | O |
| ATOM | 2594 | O | HOH | W | 19 | 61.756 | 9.877 | 125.515 | 1.00 | 40.50 | W | O |
| ATOM | 2595 | O | HON | W | 20 | 45.873 | 28.448 | 97.477 | 1.00 | 25.98 | W | O |
| ATOM | 2596 | O | HOH | W | 21 | 50.275 | 10.441 | 122.477 | 1.00 | 33.64 | W | O |
| ATOM | 2597 | O | HON | W | 22 | 56.828 | 39.659 | 107.746 | 1.00 | 37.53 | W | O |
| ATOM | 2598 | O | HOH | W | 23 | 56.922 | 8.730 | 101.984 | 1.00 | 51.25 | W | O |
| ATOM | 2599 | O | HOH | W | 24 | 63.090 | 12.420 | 103.873 | 1.00 | 23.17 | W | O |
| ATOM | 2600 | O | HOH | W | 25 | 78.432 | 18.341 | 111.530 | 1.00 | 33.10 | W | O |
| ATOM | 2601 | O | HOH | W | 26 | 55.278 | 7.063 | 126.543 | 1.00 | 22.51 | W | O |
| ATOM | 2602 | O | HOH | W | 27 | 71.699 | 14.944 | 110.281 | 1.00 | 31.13 | W | O |
| ATOM | 2603 | O | HON | W | 28 | 65.856 | 12.461 | 105.207 | 1.00 | 34.23 | W | O |
| ATOM | 2604 | O | HOH | W | 29 | 49.226 | 13.404 | 112.556 | 1.00 | 36.10 | W | O |
| ATOM | 2605 | O | HON | W | 30 | 45.806 | 26.797 | 113.582 | 1.00 | 25.06 | W | O |
| ATOM | 2606 | O | HOH | W | 31 | 50.921 | 22.489 | 96.419 | 1.00 | 38.17 | W | O |
| ATOM | 2607 | O | HOH | W | 32 | 47.043 | 17.179 | 110.619 | 1.00 | 27.88 | W | O |
| ATOM | 2608 | O | HOH | W | 33 | 50.505 | 31.197 | 136.475 | 1.00 | 33.10 | W | O |
| ATOM | 2609 | O | HOH | W | 34 | 47.341 | 22.271 | 121.211 | 1.00 | 20.31 | W | O |
| ATOM | 2610 | O | HOH | W | 35 | 61.912 | 15.434 | 101.221 | 1.00 | 22.75 | W | O |
| ATOM | 2611 | O | HOH | W | 36 | 70.118 | 14.389 | 108.156 | 1.00 | 55.54 | W | O |
| ATOM | 2612 | O | HOH | W | 37 | 60.170 | 4.215 | 113.980 | 1.00 | 40.21 | W | O |
| ATOM | 2613 | O | HOH | W | 38 | 50.540 | 23.541 | 133.215 | 1.00 | 2.46 | W | O |
| ATOM | 2614 | O | HOH | W | 39 | 48.871 | 4.037 | 141.580 | 1.00 | 13.61 | W | O |
| ATOM | 2615 | O | HOH | W | 40 | 59.625 | 29.594 | 154.497 | 1.00 | 17.76 | W | O |
| ATOM | 2616 | O | HOH | W | 41 | 58.300 | 23.513 | 135.522 | 1.00 | 22.04 | W | O |
| ATOM | 2617 | O | HOH | W | 42 | 64.850 | 17.232 | 147.313 | 1.00 | 21.84 | W | O |
| ATOM | 2618 | O | HOH | W | 43 | 49.326 | 3.982 | 150.548 | 1.00 | 24.48 | W | O |
| ATOM | 2619 | O | HOH | W | 45 | 63.774 | 19.112 | 148.870 | 1.00 | 12.98 | W | O |
| ATOM | 2620 | O | HON | W | 46 | 55.863 | 15.629 | 163.760 | 1.00 | 28.09 | W | O |
| ATOM | 2621 | O | HON | W | 47 | 53.389 | 24.883 | 133.848 | 1.00 | 20.87 | W | O |
| ATOM | 2622 | O | HOH | W | 48 | 47.215 | 9.463 | 133.451 | 1.00 | 38.63 | W | O |
| ATOM | 2623 | O | HOH | W | 49 | 54.340 | 11.753 | 153.786 | 1.00 | 25.99 | W | O |
| ATOM | 2624 | O | HOH | W | 50 | 61.472 | 23.563 | 144.309 | 1.00 | 18.90 | W | O |
| ATOM | 2625 | O | HOH | W | 51 | 63.912 | 24.339 | 144.017 | 1.00 | 19.40 | W | O |
| ATOM | 2626 | O | HOH | W | 52 | 58.245 | 29.246 | 145.388 | 1.00 | 26.64 | W | O |
| ATOM | 2627 | O | HOH | W | 53 | 63.745 | 27.746 | 148.959 | 1.00 | 20.88 | W | O |
| ATOM | 2628 | O | HOH | W | 54 | 51.102 | 26.318 | 167.875 | 1.00 | 28.44 | W | O |
| ATOM | 2629 | O | HON | W | 55 | 52.106 | 25.451 | 162.757 | 1.00 | 26.05 | W | O |
| ATOM | 2630 | O | HOH | W | 56 | 62.401 | 12.111 | 136.745 | 1.00 | 22.15 | W | O |
| ATOM | 2631 | O | HOH | W | 57 | 45.079 | 38.240 | 157.288 | 1.00 | 46.25 | W | O |
| ATOM | 2632 | O | HOH | W | 58 | 64.533 | 23.652 | 138.613 | 1.00 | 23.45 | W | O |
| ATOM | 2633 | O | HOH | W | 59 | 32.586 | 28.259 | 147.755 | 1.00 | 53.44 | W | O |
| ATOM | 2634 | O | HOH | W | 60 | 60.965 | 22.491 | 159.169 | 1.00 | 36.88 | W | O |
| ATOM | 2635 | O | HOH | W | 61 | 56.540 | 16.675 | 157.926 | 1.00 | 22.67 | W | O |
| ATOM | 2636 | O | HOH | W | 62 | 49.724 | 1.457 | 152.163 | 1.00 | 31.25 | W | O |
| ATOM | 2637 | O | HOH | W | 63 | 66.979 | 17.779 | 135.834 | 1.00 | 30.64 | W | O |
| ATOM | 2638 | O | HOH | W | 64 | 53.679 | 7.411 | 152.678 | 1.00 | 24.59 | W | O |
| ATOM | 2639 | O | HOH | W | 65 | 55.862 | 13.242 | 157.045 | 1.00 | 22.19 | W | O |
| ATOM | 2640 | O | HOH | W | 66 | 60.317 | 27.514 | 146.861 | 1.00 | 50.28 | W | O |
| ATOM | 2641 | O | HOH | W | 67 | 48.564 | 34.049 | 142.813 | 1.00 | 37.03 | W | O |
| ATOM | 2642 | O | HOH | W | 68 | 47.734 | 34.009 | 159.841 | 1.00 | 57.14 | W | O |
| ATOM | 2643 | O | HOH | W | 69 | 56.949 | 31.429 | 147.112 | 1.00 | 30.39 | W | O |
| ATOM | 2644 | O | HOH | W | 70 | 47.008 | 25.477 | 120.702 | 1.00 | 23.76 | W | O |
| ATOM | 2645 | O | HOH | W | 71 | 53.453 | 29.741 | 136.903 | 1.00 | 25.90 | W | O |
| ATOM | 2646 | O | HOH | W | 72 | 54.023 | 18.711 | 159.706 | 1.00 | 18.27 | W | O |
| ATOM | 2647 | O | HOH | W | 73 | 54.530 | 9.056 | 154.663 | 1.00 | 34.56 | W | O |
| ATOM | 2648 | O | HOH | W | 74 | 66.399 | 14.925 | 149.889 | 1.00 | 36.21 | W | O |
| ATOM | 2649 | O | HOH | W | 75 | 56.078 | 0.717 | 148.547 | 1.00 | 47.62 | W | O |
| ATOM | 2650 | O | HOH | W | 76 | 55.037 | 3.104 | 145.929 | 1.00 | 22.70 | W | O |
| ATOM | 2651 | O | HOH | W | 77 | 57.979 | 5.727 | 117.915 | 1.00 | 65.95 | W | O |
| ATOM | 2652 | O | HOH | W | 78 | 38.602 | 9.525 | 136.215 | 1.00 | 34.41 | W | O |
| ATOM | 2653 | O | HOH | W | 79 | 56.644 | 33.927 | 107.571 | 1.00 | 25.40 | W | O |
| ATOM | 2654 | O | HOH | W | 80 | 56.901 | 12.608 | 98.500 | 1.00 | 45.22 | W | O |
| ATOM | 2655 | O | HOH | W | 81 | 66.159 | 25.939 | 129.659 | 1.00 | 45.73 | W | O |
| ATOM | 2656 | O | HOH | W | 82 | 52.849 | 33.406 | 118.492 | 1.00 | 33.45 | W | O |
| ATOM | 2657 | O | HOH | W | 83 | 40.064 | 12.132 | 132.917 | 1.00 | 45.44 | W | O |
| ATOM | 2658 | O | HOH | W | 84 | 61.356 | 7.072 | 144.041 | 1.00 | 52.29 | W | O |
| ATOM | 2659 | O | HOH | W | 85 | 59.332 | 33.793 | 126.661 | 1.00 | 37.65 | W | O |
| ATOM | 2660 | O | HOH | W | 86 | 76.248 | 14.005 | 117.780 | 1.00 | 38.05 | W | O |
| ATOM | 2661 | O | HOH | W | 87 | 63.920 | 4.959 | 114.145 | 1.00 | 28.43 | W | O |
| ATOM | 2662 | O | HOH | W | 88 | 41.594 | 23.567 | 106.694 | 1.00 | 35.95 | W | O |
| ATOM | 2663 | O | HOH | W | 89 | 43.961 | 20.340 | 105.825 | 1.00 | 34.92 | W | O |
| ATOM | 2664 | O | HOH | W | 90 | 73.852 | 13.594 | 117.386 | 1.00 | 26.01 | W | O |
| ATOM | 2665 | O | HOH | W | 91 | 59.941 | 14.152 | 100.157 | 1.00 | 27.50 | W | O |
| ATOM | 2666 | O | HOH | W | 92 | 59.242 | 23.420 | 132.602 | 1.00 | 16.67 | W | O |
| ATOM | 2667 | O | HOH | W | 93 | 58.837 | 25.317 | 128.952 | 1.00 | 21.05 | W | O |
| ATOM | 2668 | O | HOH | W | 94 | 51.613 | 25.829 | 131.530 | 1.00 | 40.97 | W | O |
| ATOM | 2669 | O | HOH | W | 95 | 64.674 | 28.352 | 131.418 | 1.00 | 31.06 | W | O |
| ATOM | 2670 | O | HOH | W | 96 | 60.935 | 29.269 | 136.385 | 1.00 | 24.27 | W | O |
| ATOM | 2671 | O | HOH | W | 97 | 54.832 | 34.703 | 109.520 | 1.00 | 17.56 | W | O |
| ATOM | 2672 | O | HOH | W | 98 | 58.791 | 32.739 | 117.544 | 1.00 | 24.04 | W | O |
| ATOM | 2673 | O | HOH | W | 99 | 56.442 | 35.249 | 118.352 | 1.00 | 28.89 | W | O |
| ATOM | 2674 | O | HOH | W | 100 | 46.526 | 37.510 | 114.770 | 1.00 | 35.71 | W | O |
| ATOM | 2675 | O | HOH | W | 1O1 | 54.579 | 35.721 | 151.192 | 1.00 | 51.01 | W | O |
| ATOM | 2676 | O | HOH | W | 102 | 58.105 | 32.269 | 149.264 | 1.00 | 23.65 | W | O |
| ATOM | 2677 | O | HOH | W | 103 | 65.313 | 13.049 | 157.092 | 1.00 | 26.96 | W | O |
| ATOM | 2678 | O | HOH | W | 104 | 66.201 | 11.386 | 155.250 | 1.00 | 48.24 | W | O |
| ATOM | 2679 | O | HOH | W | 105 | 53.996 | 0.840 | 145.922 | 1.00 | 34.76 | W | O |
| ATOM | 2680 | O | HOH | W | 106 | 46.202 | 0.833 | 136.125 | 1.00 | 47.74 | W | O |
| ATOM | 2681 | O | HOH | W | 1O7 | 56.086 | 6.461 | 135.385 | 1.00 | 47.82 | W | O |
| ATOM | 2682 | O | HOH | W | 108 | 51.570 | 16.588 | 127.129 | 1.00 | 18.01 | W | O |
| ATOM | 2683 | O | HOH | W | 109 | 46.668 | 19.140 | 128.889 | 1.00 | 23.88 | W | O |
| ATOM | 2684 | O | HOH | W | 110 | 45.451 | 12.416 | 128.248 | 1.00 | 32.22 | W | O |
| ATOM | 2685 | O | HOH | W | 111 | 53.514 | 30.754 | 139.864 | 1.00 | 31.81 | W | O |
| ATOM | 2686 | O | HOH | W | 112 | 52.273 | 39.562 | 147.763 | 1.00 | 51.12 | W | O |
| ATOM | 2687 | O | HOH | W | 113 | 52.591 | 10.981 | 161.536 | 1.00 | 34.69 | W | O |
| ATOM | 2688 | O | HOH | W | 114 | 50.264 | 24.639 | 126.956 | 1.00 | 19.43 | W | O |
| ATOM | 2689 | O | HOH | W | 115 | 45.122 | 15.259 | 128.545 | 1.00 | 36.82 | W | O |
| ATOM | 2690 | O | HOH | W | 116 | 45.881 | 15.698 | 122.389 | 1.00 | 39.53 | W | O |
| ATOM | 2691 | O | HOH | W | 117 | 45.448 | 17.802 | 130.938 | 1.00 | 37.87 | W | O |
| ATOM | 2692 | O | HOH | W | 118 | 58.454 | 6.855 | 127.071 | 1.00 | 27.25 | W | O |
| ATOM | 2693 | O | HOH | W | 119 | 62.131 | 28.235 | 104.517 | 1.00 | 40.71 | W | O |
| ATOM | 2694 | O | HOH | W | 120 | 66.292 | 32.682 | 114.288 | 1.00 | 25.89 | W | O |
| ATOM | 2695 | O | HOH | W | 121 | 65.759 | 26.751 | 110.354 | 1.00 | 19.28 | W | O |
| END | | | | | | | | | | | | |

What we claim is:

1. A crystalline form of a polypeptide corresponding to the catalytic domain of an MMP9 protein.

2. A crystalline form of a polypeptide corresponding to the catalytic domain of an MMP9 protein in complex with an MMP9 inhibitor compound.

3. A crystalline form of a polypeptide corresponding to the active site binding region of an MMP9 protein wherein the active site binding region amino acid residues are identical or equivalent to those listed in Table 1 and the shape of the active site binding region is defined by the atomic coordinates given in Table 2 or Table 3 or by equivalent coordinates.

4. A method to determine or design the three dimensional structure of a crystal form of MMP9 by difference Fourier or molecular replacement, using the atomic coordinates of a first MMP9 crystal to model the structure of a second MMP9 crystal wherein the active site binding region amino acid residues of the first MMP9 crystal are identical or equivalent to those listed in Table 1 and the shape of the active site binding region of the first MMP9 crystal is defined by the atomic coordinates given in Table 2 or Table 3 or by equivalent coordinates.

5. An MMP9 protein designed by the method claimed in claim 4.

6. A method to select or design a chemical modulator of MMP9 by selecting or designing a modulator with a three dimensional structure that fits into the MMP9 active site binding region, wherein the active site binding region amino acid residues are identical or equivalent to those listed in Table 1 and the shape of the active site binding region is defined by the atomic coordinates given in Table 2 or Table 3 or by equivalent coordinates.

7. A modulator of MMP9 selected or designed by the method claimed in claim 6, or a pharmaceutically acceptable salt or in vivo hydrolysable ester thereof.

8. Use of an MMP9 modulator selected or designed by the method claimed in claim 6, or a pharmaceutically acceptable salt or in vivo hydrolysable ester thereof, to treat a metalloproteinase mediated disease or condition.

9. A method of treating a metalloproteinase mediated disease or condition which comprises administering to a warm-blooded animal a therapeutically effective amount of a modulator of MMP9 or a pharmaceutically acceptable salt or in vivo hydrolysable ester thereof, wherein the MMP9 modulator is selected or designed by the method claimed in claim 6.


SEQUENCE LISTING (2 sequences)

SEQ ID NO: 1 - protein, 660 aa, Homo sapiens
MEALMARGALTGPLRALCLLGCLLSHAAAAPSPIIKFPGDVAPKTDKELAVQYLNTFYGCPKESCNLFVLKDTLKKMQKFFGLPQTGDLDQNTIETMRKPRCGNPDVANYNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 2 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED